SEQUENCE LISTING (216 sequences)

SEQ ID NO: 1 - DNA, 2358 bp, Fusarium verticillioides
atgctgctcaatcttcaggtcgctgccagcgctttgtcgctttctcttttaggtggattggctgaggctgctacgccatatacccttccggactgtaccaaaggacctttgagcaagaatggaatctgcgatacttcgttatctccagctaaaagagcggctgctctagttgctgctctgacgcccgaagagaaggtgggcaatctggtcaggtaaaatatacccccccccataatcactattcggagattggagctgacttaacgcagcaatgcaactggtgcaccaagaatcggacttccaaggtacaactggtggaacgaagcccttcatggcctcgctggatctccaggtggtcgctttgccgacactcctccctacgacgcggccacatcatttcccatgcctcttctcatggccgctgctttcgacgatgatctgatccacgatatcggcaacgtcgtcggcaccgaagcgcgtgcgttcactaacggcggttggcgcggagtcgacttctggacacccaacgtcaacccttttaaagatcctcgctggggtcgtggctccgaaactccaggtgaagatgcccttcatgtcagccggtatgctcgctatatcgtcaggggtctcgaaggcgataaggagcaacgacgtattgttgctacctgcaagcactatgctggaaacgactttgaggactggggaggcttcacgcgtcacgactttgatgccaagattactcctcaggacttggctgagtactacgtcaggcctttccaggagtgcacccgtgatgcaaaggttggttccatcatgtgcgcctacaatgccgtgaacggcattcccgcatgcgcaaactcgtatctgcaggagacgatcctcagagggcactggaactggacgcgcgataacaactggatcactagtgattgtggcgccatgcaggatatctggcagaatcacaagtatgtcaagaccaacgctgaaggtgcccaggtagcttttgagaacggcatggattctagctgcgagtatactactaccagcgatgtctccgattcgtacaagcaaggcctcttgactgagaagctcatggatcgttcgttgaagcgccttttcgaagggcttgttcatactggtttctttgacggtgccaaagcgcaatggaactcgctcagttttgcggatgtcaacaccaaggaagctcaggatcttgcactcagatctgctgtggagggtgctgttcttcttaagaatgacggcactttgcctctgaagctcaagaagaaggatagtgttgcaatgatcggattctgggccaacgatacttccaagctgcagggtggttacagtggacgtgctccgttcctccacagcccgctttatgcagctgagaagcttggtcttgacaccaacgtggcttggggtccgacactgcagaacagctcatctcatgataactggaccaccaatgctgttgctgcggcgaagaagtctgattacattctctactttggtggtcttgacgcctctgctgctggcgaggacagagatcgtgagaaccttgactggcctgagagccagctgacccttcttcagaagctctctagtctcggcaagccactggttgttatccagcttggtgatcaagtcgatgacaccgctcttttgaagaacaagaagattaacagtattctttgggtcaattaccctggtcaggatggcggcactgcagtcatggacctgctcactggacgaaagagtcctgctggccgactacccgtcacgcaatatcccagtaaatacactgagcagattggcatgactgacatggacctcagacctaccaagtcgttgccagggagaacttatcgctggtactcaactccagttcttccctacggctttggcctccactacaccaagttccaagccaagttcaagtccaacaagttgacgtttgacatccagaagcttctcaagggctgcagtgctcaatactccgatacttgcgcgctgccccccatccaagttagtgtcaagaacaccggccgcattacctccgactttgtctctctggtctttatcaagagtgaagttggacctaagccttaccctctcaagacccttgcggcttatggtcgcttgcatgatgtcgcgccttcatcgacgaaggatatctcactggagtggacgttggataacattgcgcgacggggagagaatggtgatttggttgtttatcctgggacttacactctgttgctggatgagcctacgcaagccaagatccaggttacgctgactggaaagaaggctattttggataagtggcctcaagaccccaagtctgcgtaa

SEQ ID NO: 2 - protein, 766 aa, Fusarium verticillioides
MLLNLQVAASALSLSLLGGLAEAATPYTLPDCTKGPLSKNGICDTSLSPAKRAAALVAALTPEEKVGNLVSNATGAPRIGLPRYNWWNEALHGLAGSPGGRFADTPPYDAATSFPMPLLMAAAFDDDLIHDIGNVVGTEARAFTNGGWRGVDFWTPNVNPFKDPRWGRGSETPGEDALHVSRYARYIVRGLEGDKEQRRIVATCKHYAGNDFEDWGGFTRHDFDAKITPQDLAEYYVRPFQECTRDAKVGSIMCAYNAVNGIPACANSYLQETILRGHWNWTRDNNWITSDCGAMQDIWQNHKYVKTNAEGAQVAFENGMDSSCEYTTTSDVSDSYKQGLLTEKLMDRSLKRLFEGLVHTGFFDGAKAQWNSLSFADVNTKEAQDLALRSAVEGAVLLKNDGTLPLKLKKKDSVAMIGFWANDTSKLQGGYSGRAPFLHSPLYAAEKLGLDTNVAWGPTLQNSSSHDNWTTNAVAAAKKSDYILYFGGLDASAAGEDRDRENLDWPESQLTLLQKLSSLGKPLVVIQLGDQVDDTALLKNKKINSILWVNYPGQDGGTAVMDLLTGRKSPAGRLPVTQYPSKYTEQIGMTDMDLRPTKSLPGRTYRWYSTPVLPYGFGLHYTKFQAKFKSNKLTFDIQKLLKGCSAQYSDTCALPPIQVSVKNTGRITSDFVSLVFIKSEVGPKPYPLKTLAAYGRLHDVAPSSTKDISLEWTLDNIARRGENGDLVVYPGTYTLLLDEPTQAKIQVTLTGKKAILDKWPQDPKSA

SEQ ID NO: 3 - DNA, 1338 bp, Penicillium funiculosum
atgcttcagcgatttgcttatattttaccactggctctattgagtgttggagtgaaagccgacaacccctttgtgcagagcatctacaccgctgatccggcaccgatggtatacaatgaccgcgtttatgtcttcatggaccatgacaacaccggagctacctactacaacatgacagactggcatctgttctcgtcagcagatatggcgaattggcaagatcatggcattccaatgagcctggccaatttcacctgggccaacgcgaatgcgtgggccccgcaagtcatccctcgcaacggccaattctacttttatgctcctgtccgacacaacgatggttctatggctatcggtgtgggagtgagcagcaccatcacaggtccataccatgatgctatcggcaaaccgctagtagagaacaacgagattgatcccaccgtgttcatcgacgatgacggtcaggcatacctgtactggggaaatccagacctgtggtacgtcaaattgaaccaagatatgatatcgtacagcgggagccctactcagattccactcaccacggctggatttggtactcgaacgggcaatgctcaacggccgaccacttttgaagaagctccatgggtatacaaacgcaacggcatctactatatcgcctatgcagccgattgttgttctgaggatattcgctactccacgggaaccagtgccactggtccgtggacttatcgaggcgtcatcatgccgacccaaggtagcagcttcaccaatcacgagggtattatcgacttccagaacaactcctactttttctatcacaacggcgctcttcccggcggaggcggctaccaacgatctgtatgtgtggagcaattcaaatacaatgcagatggaaccattccgacgatcgaaatgaccaccgccggtccagctcaaattgggactctcaacccttacgtgcgacaggaagccgaaacggcggcatggtcttcaggcatcactacggaggtttgtagcgaaggcggaattgacgtcgggtttatcaacaatggcgattacatcaaagttaaaggcgtagctttcggttcaggagcccattctttctcagcgcgggttgcttctgcaaatagcggcggcactattgcaatacacctcggaagcacaactggtacgctcgtgggcacttgtactgtccccagcactggcggttggcagacttggactaccgttacctgttctgtcagtggcgcatctgggacccaggatgtgtattttgttttcggtggtagcggaacaggatacctgttcaactttgattattggcagttcgcataa

SEQ ID NO: 4 - protein, 445 aa, Penicillium funiculosum
MLQRFAYILPLALLSVGVKADNPFVQSIYTADPAPMVYNDRVYVFMDHDNTGATYYNMTDWHLFSSADMANWQDHGIPMSLANFTWANANAWAPQVIPRNGQFYFYAPVRHNDGSMAIGVGVSSTITGPYHDAIGKPLVENNEIDPTVFIDDDGQAYLYWGNPDLWYVKLNQDMISYSGSPTQIPLTTAGFGTRTGNAQRPTTFEEAPWVYKRNGIYYIAYAADCCSEDIRYSTGTSATGPWTYRGVIMPTQGSSFTNHEGIIDFQNNSYFFYHNGALPGGGGYQRSVCVEQFKYNADGTIPTIEMTTAGPAQIGTLNPYVRQEAETAAWSSGITTEVCSEGGIDVGFINNGDYIKVKGVAFGSGAHSFSARVASANSGGTIAIHLGSTTGTLVGTCTVPSTGGWQTWTTVTCSVSGASGTQDVYFVFGGSGTGYLFNFDYWQFA

SEQ ID NO: 5 - DNA, 1593 bp, Fusarium verticillioides
atgaaggtatactggctcgtggcgtgggccacttctttgacgccggcactggctggcttgattggacaccgtcgcgccaccaccttcaacaatcctatcatctactcagactttccagataacgatgtattcctcggtccagataactactactacttctctgcttccaacttccacttcagcccaggagcacccgttttgaagtctaaagatctgctaaactgggatctcatcggccattcaattccccgcctgaactttggcgacggctatgatcttcctcctggctcacgttattaccgtggaggtacttgggcatcatccctcagatacagaaagagcaatggacagtggtactggatcggctgcatcaacttctggcagacctgggtatacactgcctcatcgccggaaggtccatggtacaacaagggaaacttcggtgataacaattgctactacgacaatggcatactgatcgatgacgatgataccatgtatgtcgtatacggttccggtgaggtcaaagtatctcaactatctcaggacggattcagccaggtcaaatctcaggtagttttcaagaacactgatattggggtccaagacttggagggtaaccgcatgtacaagatcaacgggctctactatatcctaaacgatagcccaagtggcagtcagacctggatttggaagtcgaaatcaccctggggcccttatgagtctaaggtcctcgccgacaaagtcaccccgcctatctctggtggtaactcgccgcatcagggtagtctcataaagactcccaatggtggctggtacttcatgtcattcacttgggcctatcctgccggccgtcttccggttcttgcaccgattacgtggggtagcgatggtttccccattcttgtcaagggtgctaatggcggatggggatcatcttacccaacacttcctggcacggatggtgtgacaaagaattggacaaggactgataccttccgcggaacctcacttgctccgtcctgggagtggaaccataatccggacgtcaactccttcactgtcaacaacggcctgactctccgcactgctagcattacgaaggatatttaccaggcgaggaacacgctatctcaccgaactcatggtgatcatccaacaggaatagtgaagattgatttctctccgatgaaggacggcgaccgggccgggctttcagcgtttcgagaccaaagtgcatacatcggtattcatcgagataacggaaagttcacaatcgctacgaagcatgggatgaatatggatgagtggaacggaacaacaacagacctgggacaaataaaagccacagctaatgtgccttctggaaggaccaagatctggctgagacttcaacttgataccaacccagcaggaactggcaacactatcttttcttacagttgggatggagtcaagtatgaaacactgggtcccaacttcaaactgtacaatggttgggcattctttattgcttaccgattcggcatcttcaacttcgccgagacggctttaggaggctcgatcaaggttgagtctttcacagctgcatag

SEQ ID NO: 6 - protein, 530 aa, Fusarium verticillioides
MKVYWLVAWATSLTPALAGLIGHRRATTFNNPIIYSDFPDNDVFLGPDNYYYFSASNFHFSPGAPVLKSKDLLNWDLIGHSIPRLNFGDGYDLPPGSRYYRGGTWASSLRYRKSNGQWYWIGCINFWQTWVYTASSPEGPWYNKGNFGDNNCYYDNGILIDDDDTMYVVYGSGEVKVSQLSQDGFSQVKSQVVFKNTDIGVQDLEGNRMYKINGLYYILNDSPSGSQTWIWKSKSPWGPYESKVLADKVTPPISGGNSPHQGSLIKTPNGGWYFMSFTWAYPAGRLPVLAPITWGSDGFPILVKGANGGWGSSYPTLPGTDGVTKNWTRTDTFRGTSLAPSWEWNHNPDVNSFTVNNGLTLRTASITKDIYQARNTLSHRTHGDHPTGIVKIDFSPMKDGDRAGLSAFRDQSAYIGIHRDNGKFTIATKHGMNMDEWNGTTTDLGQIKATANVPSGRTKIWLRLQLDTNPAGTGNTIFSYSWDGVKYETLGPNFKLYNGWAFFIAYRFGIFNFAETALGGSIKVESFTAA

SEQ ID NO: 7 - DNA, 1374 bp, Fusarium verticillioides
atgcactacgctaccctcaccactttggtgctggctctgaccaccaacgtcgctgcacagcaaggcacagcaactgtcgacctctccaaaaatcatggaccggcgaaggcccttggttcaggcttcatatacggctggcctgacaacggaacaagcgtcgacacctccataccagatttcttggtaactgacatcaaattcaactcaaaccgcggcggtggcgcccaaatcccatcactgggttgggccagaggtggctatgaaggatacctcggccgcttcaactcaaccttatccaactatcgcaccacgcgcaagtataacgctgactttatcttgttgcctcatgacctctggggtgcggatggcgggcagggttcaaactccccgtttcctggcgacaatggcaattggactgagatggagttattctggaatcagcttgtgtctgacttgaaggctcataatatgctggaaggtcttgtgattgatgtttggaatgagcctgatattgatatcttttgggatcgcccgtggtcgcagtttcttgagtattacaatcgcgcgaccaaactacttcggtgagtctactactgatccatacgtatttacagtgagctgactggtcgaattagaaaaacacttcccaaaactcttctcagtggcccagccatggcacattctcccattctgtccgatgataaatggcatacctggcttcaatcagtagcgggtaacaagacagtccctgatatttactcctggcatcagattggcgcttgggaacgtgagccggacagcactatccccgactttaccaccttgcgggcgcaatatggcgttcccgagaagccaattgacgtcaatgagtacgctgcacgcgatgagcaaaatccagccaactccgtctactacctctctcaactagagcgtcataaccttagaggtcttcgcgcaaactggggtagcggatctgacctccacaactggatgggcaacttgatttacagcactaccggtacctcggaggggacttactaccctaatggtgaatggcaggcttacaagtactatgcggccatggcagggcagagacttgtgaccaaagcatcgtcggacttgaagtttgatgtctttgccactaagcaaggccgtaagattaagattatagccggcacgaggaccgttcaagcaaagtataacatcaaaatcagcggtttggaagtagcaggacttcctaagatgggtacggtaaaggtccggacttatcggttcgactgggctgggccgaatggaaaggttgacgggcctgttgatttgggggagaagaagtatacttattcggccaatacggtgagcagcccctctacttga

SEQ ID NO: 8 - protein, 439 aa, Fusarium verticillioides
MHYATLTTLVLALTTNVAAQQGTATVDLSKNHGPAKALGSGFIYGWPDNGTSVDTSIPDFLVTDIKFNSNRGGGAQIPSLGWARGGYEGYLGRFNSTLSNYRTTRKYNADFILLPHDLWGADGGQGSNSPFPGDNGNWTEMELFWNQLVSDLKAHNMLEGLVIDVWNEPDIDIFWDRPWSQFLEYYNRATKLLRKTLPKTLLSGPAMAHSPILSDDKWHTWLQSVAGNKTVPDIYSWHQIGAWEREPDSTIPDFTTLRAQYGVPEKPIDVNEYAARDEQNPANSVYYLSQLERHNLRGLRANWGSGSDLHNWMGNLIYSTTGTSEGTYYPNGEWQAYKYYAAMAGQRLVTKASSDLKFDVFATKQGRKIKIIAGTRTVQAKYNIKISGLEVAGLPKMGTVKVRTYRFDWAGPNGKVDGPVDLGEKKYTYSANTVSSPST

SEQ ID NO: 9 - DNA, 1350 bp, Fusarium verticillioides
atgtggctgacctccccattgctgttcgccagcaccctcctgggcctcactggcgttgctctagcagacaaccccatcgtccaagacatctacaccgcagacccagcaccaatggtctacaatggccgcgtctacctcttcacaggccatgacaacgacggctctaccgacttcaacatgacagactggcgtctcttctcgtcagcagacatggtcaactggcagcaccatggtgtccccatgagcttaaagaccttcagctgggccaacagcagagcctgggctggtcaagtcgttgcccgaaacggaaagttttacttctatgttcctgtccgtaatgccaagacgggtggaatggctattggtgtcggtgttagtaccaacatccttgggccctacactgatgcccttggaaagccattggtcgagaacaatgagatcgacccaactgtctacatcgacactgatggccaggcctatctctactggggcaaccctggattgtactacgtcaagctcaaccaagacatgctctcctacagtggtagcatcaacaaagtatcgctcacaacagctggattcggcagccgcccgaacaacgcgcagcgtcctactactttcgaggaaggaccgtggctgtacaagcgtggaaatctctactacatgatctacgcagccaactgctgttccgaggacattcgctactcaactggacccagcgccactggaccttggacttaccgcggtgtcgtgatgaacaaggcgggtcgaagcttcaccaaccatcctggcatcatcgactttgagaacaactcgtacttcttttaccacaatggcgctcttgatggaggtagcggttatactcggtctgtggctgtcgagagcttcaagtatggttcggacggtctgatccccgagatcaagatgactacgcaaggcccagcgcagctcaagtctctgaacccatatgtcaagcaggaggccgagactatcgcctggtctgagggtatcgagactgaggtctgcagcgaaggtggtctcaacgttgctttcatcgacaatggtgactacatcaaggtcaagggagtcgactttggcagcaccggtgcaaagacgttcagcgcccgtgttgcttccaacagcagcggaggcaagattgagcttcgacttggtagcaagaccggtaagttggttggtacctgcacggtaacgactacgggaaactggcagacttataagactgtggattgccccgtcagtggtgctactggtacgagcgatctattctttgtcttcacgggctctgggtctggctctctgttcaacttcaactggtggcagtttagctaa

SEQ ID NO: 10 - protein, 449 aa, Fusarium verticillioides
MWLTSPLLFASTLLGLTGVALADNPIVQDIYTADPAPMVYNGRVYLFTGHDNDGSTDFNMTDWRLFSSADMVNWQHHGVPMSLKTFSWANSRAWAGQVVARNGKFYFYVPVRNAKTGGMAIGVGVSTNILGPYTDALGKPLVENNEIDPTVYIDTDGQAYLYWGNPGLYYVKLNQDMLSYSGSINKVSLTTAGFGSRPNNAQRPTTFEEGPWLYKRGNLYYMIYAANCCSEDIRYSTGPSATGPWTYRGVVMNKAGRSFTNHPGIIDFENNSYFFYHNGALDGGSGYTRSVAVESFKYGSDGLIPEIKMTTQGPAQLKSLNPYVKQEAETIAWSEGIETEVCSEGGLNVAFIDNGDYIKVKGVDFGSTGAKTFSARVASNSSGGKIELRLGSKTGKLVGTCTVTTTGNWQTYKTVDCPVSGATGTSDLFFVFTGSGSGSLFNFNWWQFS

SEQ ID NO: 11 - DNA, 1725 bp, Fusarium verticillioides
atgcgcttctcttggctattgtgcccccttctagcgatgggaagtgctcttcctgaaacgaagacggatgtttcgacatacaccaaccctgtccttccaggatggcactcggatccatcgtgtatccagaaagatggcctctttctctgcgtcacttcaacattcatctccttcccaggtcttcccgtctatgcctcaagggatctagtcaactggcgtctcatcagccatgtctggaaccgcgagaaacagttgcctggcattagctggaagacggcaggacagcaacagggaatgtatgcaccaaccattcgataccacaagggaacatactacgtcatctgcgaatacctgggcgttggagatattattggtgtcatcttcaagaccaccaatccgtgggacgagagtagctggagtgaccctgttaccttcaagccaaatcacatcgaccccgatctgttctgggatgatgacggaaaggtttattgtgctacccatggcatcactctgcaggagattgatttggaaactggagagcttagcccggagcttaatatctggaacggcacaggaggtgtatggcctgagggtccccatatctacaagcgcgacggttactactatctcatgattgccgagggtggaactgccgaagaccacgctatcacaatcgctcgggcccgcaagatcaccggcccctatgaagcctacaataacaacccaatcttgaccaaccgcgggacatctgagtacttccagactgtcggtcacggtgatctgttccaagataccaagggcaactggtggggtctttgtcttgctactcgcatcacagcacagggagtttcacccatgggccgtgaagctgttttgttcaatggcacatggaacaagggcgaatggcccaagttgcaaccagtacgaggtcgcatgcctggaaacctcctcccaaagccgacgcgaaacgttcccggagatgggcccttcaacgctgacccagacaactacaacttgaagaagactaagaagatccctcctcactttgtgcaccatagagtcccaagagacggtgccttctctttgtcttccaagggtctgcacatcgtgcctagtcgaaacaacgttaccggtagtgtgttgccaggagatgagattgagctatcaggacagcgaggtctagctttcatcggacgccgccaaactcacactctgttcaaatatagtgttgatatcgacttcaagcccaagtccgatgatcaggaagctggaatcaccgttttccgcacgcagttcgaccatatcgatcttggcattgttcgtcttcctacaaaccaaggcagcaacaagaaatctaagcttgccttccgattccgggccacaggagctcagaatgttcctgcaccgaaggtagtaccggtccccgatggctgggagaagggcgtaatcagtctacatatcgaggcagccaacgcgacgcactacaaccttggagcttcgagccacagaggcaagactctcgacatcgcgacagcatcagcaagtcttgtgagtggaggcacgggttcatttgttggtagtttgcttggaccttatgctacctgcaacggcaaaggatctggagtggaatgtcccaagggaggtgatgtctatgtgacccaatggacttataagcccgtggcacaagagattgatcatggtgtttttgtgaaatcagaattgtag

SEQ ID NO: 12 - protein, 574 aa, Fusarium verticillioides
MRFSWLLCPLLAMGSALPETKTDVSTYTNPVLPGWHSDPSCIQKDGLFLCVTSTFISFPGLPVYASRDLVNWRLISHVWNREKQLPGISWKTAGQQQGMYAPTIRYHKGTYYVICEYLGVGDIIGVIFKTTNPWDESSWSDPVTFKPNHIDPDLFWDDDGKVYCATHGITLQEIDLETGELSPELNIWNGTGGVWPEGPHIYKRDGYYYLMIAEGGTAEDHAITIARARKITGPYEAYNNNPILTNRGTSEYFQTVGHGDLFQDTKGNWWGLCLATRITAQGVSPMGREAVLFNGTWNKGEWPKLQPVRGRMPGNLLPKPTRNVPGDGPFNADPDNYNLKKTKKIPPHFVHHRVPRDGAFSLSSKGLHIVPSRNNVTGSVLPGDEIELSGQRGLAFIGRRQTHTLFKYSVDIDFKPKSDDQEAGITVFRTQFDHIDLGIVRLPTNQGSNKKSKLAFRFRATGAQNVPAPKVVPVPDGWEKGVISLHIEAANATHYNLGASSHRGKTLDIATASASLVSGGTGSFVGSLLGPYATCNGKGSGVECPKGGDVYVTQWTYKPVAQEIDHGVFVKSEL

SEQ ID NO: 13 - DNA, 2251 bp, Podospora anserina
atgatccacctcaagccagccctcgcggcgttgttggcgctgtcgacgcaatgtgtggctattgatttgtttgtcaagtcttcgggggggaataagacgactgatatcatgtatggtcttatgcacgaggtatgtgttttgcgagatctcccttttgtttttgcgcactgctgacatggagactgcaaacaggatatcaacaactccggcgacggcggcatctacgccgagctaatctccaaccgcgcgttccaagggagtgagaagttcccctccaacctcgacaactggagccccgtcggtggcgctacccttacccttcagaagcttgccaagcccctttcctctgcgttgccttactccgtcaatgttgccaaccccaaggagggcaagggcaagggcaaggacaccaaggggaagaaggttggcttggccaatgctgggttttggggtatggatgtcaagaggcagaagtacactggtagcttccacgttactggtgagtacaagggtgactttgaggttagcttgcgcagcgcgattaccggggagacctttggcaagaaggtggtgaagggtgggagtaagaaggggaagtggaccgagaaggagtttgagttggtgcctttcaaggatgcgcccaacagcaacaacacctttgttgtgcagtgggatgccgaggtatgtgcttctttgatattggctgagatagaagttgggttgacatgatgtggtgcagggcgcaaaggacggatctttggatctcaacttgatcagcttgttccctccgacattcaagggaaggaagaatgggctgagaattgatcttgcgcagacgatggttgagctcaagccggtaagtcctctctagtcagaaaagtagagcctttgttaacgcttgacagaccttcttgcgcttccccggtggcaacatgctcgagggtaacaccttggacacttggtggaagtggtacgagaccattggccctctgaaggatcgcccgggcatggctggtgtctgggagtaccagcaaacccttggcttgggtctggtcgagtacatggagtgggccgatgacatgaacttggagcccagtatgtgatcccattttctggagtgacttctcttgctaacgtatccacagttgtcggtgtcttcgctggtcttgccctcgatggctcgttcgttcccgaatccgagatgggatgggtcatccaacaggctctcgacgaaatcgagttcctcactggcgatgctaagaccaccaaatggggtgccgtccgcgcgaagcttggtcaccccaagccttggaaggtcaagtgggttgagatcggtaacgaggattggcttgccggacgccctgctggcttcgagtcgtacatcaactaccgcttccccatgatgatgaaggccttcaacgaaaagtaccccgacatcaagatcatcgcctcgccctccatcttcgacaacatgacaatccccgcgggtgctgccggtgatcaccacccgtacctgactcccgatgagttcgttgagcgattcgccaagttcgataacttgagcaaggataacgtgacgctcatcggcgaggctgcgtcgacgcatcctaacggtggtatcgcttgggagggagatctcatgcccttgccttggtggggcggcagtgttgctgaggctatcttcttgatcagcactgagagaaacggtgacaagatcatcggtgctacttacgcgcctggtcttcgcagcttggaccgctggcaatggagcatgacctgggtgcagcatgccgccgacccggccctcaccactcgctcgaccagttggtatgtctggagaatcctcgcccaccacatcatccgtgagacgctcccggtcgatgccccggccggcaagcccaactttgaccctctgttctacgttgccggaaagagcgagagtggcaccggtatcttcaaggctgccgtctacaactcgactgaatcgatcccggtgtcgttgaagtttgatggtctcaacgagggagcggttgccaacttgacggtgcttactgggccggaggatccgtatggatacaacgaccccttcactggtatcaatgttgtcaaggagaagaccaccttcatcaaggccggaaagggcggcaagttcaccttcaccctgccgggcttgagtgttgctgtgttggagacggccgacgcggtcaagggtggcaagggaaagggcaagggcaagggaaagggtaactga

SEQ ID NO: 14 - protein, 676 aa, Podospora anserina
MIHLKPALAALLALSTQCVAIDLFVKSSGGNKTTDIMYGLMHEDINNSGDGGIYAELISNRAFQGSEKFPSNLDNWSPVGGATLTLQKLAKPLSSALPYSVNVANPKEGKGKGKDTKGKKVGLANAGFWGMDVKRQKYTGSFHVTGEYKGDFEVSLRSAITGETFGKKVVKGGSKKGKWTEKEFELVPFKDAPNSNNTFVVQWDAEGAKDGSLDLNLISLFPPTFKGRKNGLRIDLAQTMVELKPTFLRFPGGNMLEGNTLDTWWKWYETIGPLKDRPGMAGVWEYQQTLGLGLVEYMEWADDMNLEPIVGVFAGLALDGSFVPESEMGWVIQQALDEIEFLTGDAKTTKWGAVRAKLGHPKPWKVKWVEIGNEDWLAGRPAGFESYINYRFPMMMKAFNEKYPDIKIIASPSIFDNMTIPAGAAGDHHPYLTPDEFVERFAKFDNLSKDNVTLIGEAASTHPNGGIAWEGDLMPLPWWGGSVAEAIFLISTERNGDKIIGATYAPGLRSLDRWQWSMTWVQHAADPALTTRSTSWYVWRILAHHIIRETLPVDAPAGKPNFDPLFYVAGKSESGTGIFKAAVYNSTESIPVSLKFDGLNEGAVANLTVLTGPEDPYGYNDPFTGINVVKEKTTFIKAGKGGKFTFTLPGLSVAVLETADAVKGGKGKGKGKGKGN

SEQ ID NO: 15 - DNA, 1023 bp, Gibberella zeae
atgaagtccaagttgttattcccactcctctctttcgttggtcaaagtcttgccaccaacgacgactgtcctctcatcactagtagatggactgcggatccttcggctcatgtctttaacgacaccttgtggctctacccgtctcatgacatcgatgctggatttgagaatgatcctgatggaggccagtacgccatgagagattaccatgtctactctatcgacaagatctacggttccctgccggtcgatcacggtacggccctgtcagtggaggatgtcccctgggcctctcgacagatgtgggctcctgacgctgcccacaagaacggcaaatactacctatacttccctgccaaagacaaggatgatatcttcagaatcggcgttgctgtctcaccaacccccggcggaccattcgtccccgacaagagttggatccctcacactttcagcatcgaccccgccagtttcgtcgatgatgatgacagagcctacttggcatggggtggtatcatgggtggccagcttcaacgatggcaggataagaacaagtacaacgaatctggcactgagccaggaaacggcaccgctgccttgagccctcagattgccaagctgagcaaggacatgcacactctggcagagaagcctcgcgacatgctcattcttgaccccaagactggcaagccgctcctttctgaggatgaagaccgacgcttcttcgaaggaccctggattcacaagcgcaacaagatttactacctcacctactctactggcacaacccactatcttgtctatgcgacttcaaagaccccctatggtccttacacctaccagggcagaattctggagccagttgatggctggactactcactctagtatcgtcaagtaccagggtcagtggtggctattttatcacgatgccaagacatctggcaaggactatcttcgccaggtaaaggctaagaagatttggtacgatagcaaaggaaagatcttgacaaagaagccttga

SEQ ID NO: 16 - protein, 340 aa, Gibberella zeae
MKSKLLFPLLSFVGQSLATNDDCPLITSRWTADPSAHVFNDTLWLYPSHDIDAGFENDPDGGQYAMRDYHVYSIDKIYGSLPVDHGTALSVEDVPWASRQMWAPDAAHKNGKYYLYFPAKDKDDIFRIGVAVSPTPGGPFVPDKSWIPHTFSIDPASFVDDDDRAYLAWGGIMGGQLQRWQDKNKYNESGTEPGNGTAALSPQIAKLSKDMHTLAEKPRDMLILDPKTGKPLLSEDEDRRFFEGPWIHKRNKIYYLTYSTGTTHYLVYATSKTPYGPYTYQGRILEPVDGWTTHSSIVKYQGQWWLFYHDAKTSGKDYLRQVKAKKIWYDSKGKILTKKP

SEQ ID NO: 17 - DNA, 1047 bp, Fusarium oxysporum
atgcagctcaagtttctgtcttcagcattgctgttctctctgaccagcaaatgcgctgcgcaagacactaatgacattcctcccctgatcaccgacctctggtccgcagatccctcggctcatgttttcgaaggcaagctctgggtttacccatctcacgacatcgaagccaatgttgtcaacggcacaggaggcgctcaatacgccatgagggattaccatacctactccatgaagagcatctatggtaaagatcccgttgtcgaccacggcgtcgctctctcagtcgatgacgttccctgggcgaagcagcaaatgtgggctcctgacgcagctcataagaacggcaaatattatctgtacttccccgccaaggacaaggatgagatcttcagaattggagttgctgtctccaacaagcccagcggtcctttcaaggccgacaagagctggatccctggcacgtacagtatcgatcctgctagctacgtcgacactgataacgaggcctacctcatctggggcggtatctggggcggccagctccaagcctggcaggataaaaagaactttaacgagtcgtggattggagacaaggctgctcctaacggcaccaatgccctatctcctcagatcgccaagctaagcaaggacatgcacaagatcaccgaaacaccccgcgatctcgtcattctcgcccccgagacaggcaagcctcttcaggctgaggacaacaagcgacgattcttcgagggcccttggatccacaagcgcggcaagctttactacctcatgtactccaccggtgatacccacttccttgtctacgctacttccaagaacatctacggtccttatacctaccggggcaagattcttgatcctgttgatgggtggactactcatggaagtattgttgagtataagggacagtggtggcttttctttgctgatgcgcatacgtctggtaaggattaccttcgacaggtgaaggcgaggaagatctggtatgacaagaacggcaagatcttgcttcaccgtccttag

SEQ ID NO: 18 - protein, 348 aa, Fusarium oxysporum
MQLKFLSSALLFSLTSKCAAQDTNDIPPLITDLWSADPSAHVFEGKLWVYPSHDIEANVVNGTGGAQYAMRDYHTYSMKSIYGKDPVVDHGVALSVDDVPWAKQQMWAPDAAHKNGKYYLYFPAKDKDEIFRIGVAVSNKPSGPFKADKSWIPGTYSIDPASYVDTDNEAYLIWGGIWGGQLQAWQDKKNFNESWIGDKAAPNGTNALSPQIAKLSKDMHKITETPRDLVILAPETGKPLQAEDNKRRFFEGPWIHKRGKLYYLMYSTGDTHFLVYATSKNIYGPYTYRGKILDPVDGWTTHGSIVEYKGQWWLFFADAHTSGKDYLRQVKARKIWYDKNGKILLHRP

SEQ ID NO: 19 - DNA, 1677 bp, Aspergillus fumigatus
atggcagctccaagtttatcctaccccacaggtatccaatcgtataccaatcctctcttccctggttggcactccgatcccagctgtgcctacgtagcggagcaagacacctttttctgcgtgacgtccactttcattgccttccccggtcttcctctttatgcaagccgagatctgcagaactggaaactggcaagcaatattttcaatcggcccagccagatccctgatcttcgcgtcacggatggacagcagtcgggtatctatgcgcccactctgcgctatcatgagggccagttctacttgatcgtttcgtacctgggcccgcagactaagggcttgctgttcacctcgtctgatccgtacgacgatgccgcgtggagcgatccgctcgaattcgcggtacatggcatcgacccggatatcttctgggatcacgacgggacggtctatgtcacgtccgccgaggaccagatgattaagcagtacacactcgatctgaagacgggggcgattggcccggttgactacctctggaacggcaccggaggagtctggcccgagggcccgcacatttacaagagagacggatactactacctcatgatcgcagagggaggtaccgagctcggccactcggagaccatggcgcgatctagaacccggacaggtccctgggagccatacccgcacaatccgctcttgtcgaacaagggcacctcggagtacttccagactgtgggccatgcggacttgttccaggatgggaacggcaactggtgggccgtggcgttgagcacccgatcagggcctgcatggaagaactatcccatgggtcgggagacggtgctcgcccccgccgcttgggagaagggtgagtggcctgtcattcagcctgtgagaggccaaatgcaggggccgtttccaccaccaaataagcgagttcctcgcggcgagggcggatggatcaagcaacccgacaaagtggatttcaggcccggatcgaagataccggcgcacttccagtactggcgatatcccaagacagaggattttaccgtctcccctcggggccacccgaatactcttcggctcacaccctccttttacaacctcaccggaactgcggacttcaagccggatgatggcctgtcgcttgttatgcgcaaacagaccgacaccttgttcacgtacactgtggacgtgtcttttgaccccaaggttgccgatgaagaggcgggtgtgactgttttccttacccagcagcagcacatcgatcttggtattgtccttctccagacaaccgaggggctgtcgttgtccttccggttccgcgtggaaggccgcggtaactacgaaggtcctcttccagaagccaccgtgcctgttcccaaggaatggtgtggacagaccatccggcttgagattcaggccgtgagtgacaccgagtatgtctttgcggctgccccggctcggcaccctgcacagaggcaaatcatcagccgcgccaactcgttgattgtcagtggtgatacgggacggtttactggctcgcttgttggcgtgtatgccacgtcgaacgggggtgccggatccacgcccgcatatatcagcagatggagatacgaaggacggggccagatgattgattttggtcgagtggtcccgagctactga

SEQ ID NO: 20 - protein, 558 aa, Aspergillus fumigatus
MAAPSLSYPTGIQSYTNPLFPGWHSDPSCAYVAEQDTFFCVTSTFIAFPGLPLYASRDLQNWKLASNIFNRPSQIPDLRVTDGQQSGIYAPTLRYHEGQFYLIVSYLGPQTKGLLFTSSDPYDDAAWSDPLEFAVHGIDPDIFWDHDGTVYVTSAEDQMIKQYTLDLKTGAIGPVDYLWNGTGGVWPEGPHIYKRDGYYYLMIAEGGTELGHSETMARSRTRTGPWEPYPHNPLLSNKGTSEYFQTVGHADLFQDGNGNWWAVALSTRSGPAWKNYPMGRETVLAPAAWEKGEWPVIQPVRGQMQGPFPPPNKRVPRGEGGWIKQPDKVDFRPGSKIPAHFQYWRYPKTEDFTVSPRGHPNTLRLTPSFYNLTGTADFKPDDGLSLVMRKQTDTLFTYTVDVSFDPKVADEEAGVTVFLTQQQHIDLGIVLLQTTEGLSLSFRFRVEGRGNYEGPLPEATVPVPKEWCGQTIRLEIQAVSDTEYVFAAAPARHPAQRQIISRANSLIVSGDTGRFTGSLVGVYATSNGGAGSTPAYISRWRYEGRGQMIDFGRVVPSY

SEQ ID NO: 21 - DNA, 2320 bp, Penicillium funiculosum
atgggaaagatgtggcattcgatcttggttgtgttgggcttattgtctgtcgggcatgccatcactatcaacgtgtcccaaagtggcggcaataagaccagtcctttgcaatatggtctgatgttcgaggtaatccttctcttataccacatataaaagttgcgtcatttctaagacaagtcaaggacataaatcacggcggtgatggcggtctgtatgcagagcttgttcgaaaccgagcattccaaggtagcaccgtctatccagcaaacctcgatggatacgactcggtcaatggagcaatcctagcgcttcagaatttgacaaaccctctatcaccctccatgcctagctctctcaacgtcgccaaggggtccaacaatggaagcatcggtttcgcaaatgaaggctggtgggggatagaagtcaagccgcaaagatacgcgggctcattctacgtccagggggactatcaaggagatttcgacatctctcttcagtcgaaattgacacaagaagtcttcgcaacggcaaaagtcaggtcctcgggcaaacacgaggactgggttcaatacaagtacgagttggtgcccaaaaaggcagcatcaaacaccaataacactctgaccattacttttgactcaaaggtatgttaaattttgggtttagttcgatgtctggcaattgtcttacgagaaacgtagggattgaaagacggatccttgaacttcaacttgatcagcctatttcccccaacttacaacaatcggcccaatggcctaagaatcgacctggttgaagctatggctgaactagagggggtaagctcttacaaatcaactttatctttacgaagactaatgtgaaaacttagaaatttctgcggtttccaggcggtagcgatgtggaaggtgtacaagctccttactggtataagtggaatgaaacggtaggagatctcaaggaccgttatagtaggcccagtgcatggacgtacgaagaaagcaatggaattggcttgattgagtacatgaattggtgtgatgacatggggcttgagccgagtgagtgtattccattcagcgtcaaatccagtgttctaatcatacacatcagttcttgccgtatgggatggacattacctttcgaacgaagtgatatcggaaaacgatttgcagccatatatcgacgacaccctcaaccaactggaattcctgatgggtgccccagatacgccatatggtagttggcgtgcgtctctgggctatccgaagccgtggacgattaactacgtcgagattggaaacgaagacaatctatacgggggactagaaacatacatcgcctaccggtttcaggcatattacgacgctataacagctaaatatccccatatgacggtcatggaatctttgacggagatgcctggtccggcggccgctgcaagcgattaccatcaatattctactcctgatgggtttgtttcccagttcaactactttgatcagatgccagtcactaatagaacactgaacggtatgaaaacccccccttttttaaatatgcttttaatggtattaaccatctttcataggagagattgcaaccgtttatccaaataatcctagtaattcggtggcctggggaagcccattccccttgtatccttggtggattgggtccgttgcagaagctgttttcctaattggtgaagagaggaattcgccaaagataatcggtgctagctacgtacggaattctacttttcgagattttaacattggataagaaggactaacctcaatacaggctccaatgttcagaaatatcaacaattggcagtggtctccaacactcatcgcttttgacgctgactcgtcgcgtacaagtcgttcaacaagctggcatgtgatcaaggtatgctaattttcctcctcattcaaacccgcagatgtgagctaactttccgaagcttctctcgacaaacaaaatcacgcaaaatttacccacgacttggagtggcggtgacataggtccattatactgggtagctggacgaaacgacaatacaggatcgaacatattcaaggccgctgtttacaacagcacctcagacgtccctgtcaccgttcaatttgcaggatgcaacgcaaagagcgcaaatttgaccatcttgtcatccgacgatccgaacgcatcgaactaccctggggggcccgaagttgtgaagactgagatccagtctgtcactgcaaatgctcatggagcatttgagttcagtctcccgaacctaagtgtggctgttctcaaaacggagtaa

SEQ ID NO: 22 - protein, 642 aa, Penicillium funiculosum
MGKMWHSILVVLGLLSVGHAITINVSQSGGNKTSPLQYGLMFEDINHGGDGGLYAELVRNRAFQGSTVYPANLDGYDSVNGAILALQNLTNPLSPSMPSSLNVAKGSNNGSIGFANEGWWGIEVKPQRYAGSFYVQGDYQGDFDISLQSKLTQEVFATAKVRSSGKHEDWVQYKYELVPKKAASNTNNTLTITFDSKGLKDGSLNFNLISLFPPTYNNRPNGLRIDLVEAMAELEGKFLRFPGGSDVEGVQAPYWYKWNETVGDLKDRYSRPSAWTYEESNGIGLIEYMNWCDDMGLEPILAVWDGHYLSNEVISENDLQPYIDDTLNQLEFLMGAPDTPYGSWRASLGYPKPWTINYVEIGNEDNLYGGLETYIAYRFQAYYDAITAKYPHMTVMESLTEMPGPAAAASDYHQYSTPDGFVSQFNYFDQMPVTNRTLNGEIATVYPNNPSNSVAWGSPFPLYPWWIGSVAEAVFLIGEERNSPKIIGASYAPMFRNINNWQWSPTLIAFDADSSRTSRSTSWHVIKLLSTNKITQNLPTTWSGGDIGPLYWVAGRNDNTGSNIFKAAVYNSTSDVPVTVQFAGCNAKSANLTILSSDDPNASNYPGGPEVVKTEIQSVTANAHGAFEFSLPNLSVAVLKTE

SEQ ID NO: 23 - DNA, 739 bp, Aspergillus fumigatus
atggtttctttctcctacctgctgctggcgtgctccgccattggagctctggctgcccccgtcgaacccgagaccacctcgttcaatgagactgctcttcatgagttcgctgagcgcgccggcaccccaagctccaccggctggaacaacggctactactactccttctggactgatggcggcggcgacgtgacctacaccaatggcgccggtggctcgtactccgtcaactggaggaacgtgggcaactttgtcggtggaaagggctggaaccctggaagcgctaggtaccgagctttgtcaacgtcggatgtgcagacctgtggctgacagaagtagaaccatcaactacggaggcagcttcaaccccagcggcaatggctacctggctgtctacggctggaccaccaaccccttgattgagtactacgttgttgagtcgtatggtacatacaaccccggcagcggcggtaccttcaggggcactgtcaacaccgacggtggcacttacaacatctacacggccgttcgctacaatgctccctccatcgaaggcaccaagaccttcacccagtactggtctgtgcgcacctccaagcgtaccggcggcactgtcaccatggccaaccacttcaacgcctggagcagactgggcatgaacctgggaactcacaactaccagattgtcgccactgagggttaccagagcagcggatctgcttccatcactgtctactag

SEQ ID NO: 24 - protein, 228 aa, Aspergillus fumigatus
MVSFSYLLLACSAIGALAAPVEPETTSFNETALHEFAERAGTPSSTGWNNGYYYSFWTDGGGDVTYTNGAGGSYSVNWRNVGNFVGGKGWNPGSARTINYGGSFNPSGNGYLAVYGWTTNPLIEYYVVESYGTYNPGSGGTFRGTVNTDGGTYNIYTAVRYNAPSIEGTKTFTQYWSVRTSKRTGGTVTMANHFNAWSRLGMNLGTHNYQIVATEGYQSSGSASITVY

SEQ ID NO: 25 - DNA, 1002 bp, Aspergillus fumigatus
atgatctccatttcctcgctcagctttggactcgccgctatcgccggcgcatatgctcttccgagtgacaaatccgtcagcttagcggaacgtcagacgatcacgaccagccagacaggcacaaacaatggctactactattccttctggaccaacggtgccggatcagtgcaatatacaaatggtgctggtggcgaatatagtgtgacgtgggcgaaccagaacggtggtgactttacctgtgggaagggctggaatccagggagtgaccagtaggcaacgcccgagaactatagaagaggacgcaaagaaagcactaaactctctactagtgacattaccttctctggcagcttcaatccttccggaaatgcttacctgtccgtgtatggatggactaccaaccccctagtcgaatactacatcctcgagaactatggcagttacaatcctggctcgggcatgacgcacaagggcaccgtcaccagcgatggatccacctacgacatctatgagcaccaacaggtcaaccagccttcgatcgtcggcacggccaccttcaaccaatactggtccatccgccaaaacaagcgatccagcggcacagtcaccaccgcgaatcacttcaaggcctgggctagtctggggatgaacctgggtacccataactatcagattgtttccactgagggatatgagagcagcggtacctcgaccatcactgtctcgtctggtggttcttcttctggtggaagtggtggcagctcgtctactacttcctcaggcagctcccctactggtggctccggcagtgtaagtcttcttccatatggttgtggctttatgtgtattctgactgtgatagtgctctgctttgtggggccagtgcggtggaattggctggtctggtcctacttgctgctcttcgggcacttgccaggtttcgaactcgtactactcccagtgcttgtagtaccttcttgcagggttatatccaagtga

SEQ ID NO: 26 - protein, 286 aa, Aspergillus fumigatus
MISISSLSFGLAAIAGAYALPSDKSVSLAERQTITTSQTGTNNGYYYSFWTNGAGSVQYTNGAGGEYSVTWANQNGGDFTCGKGWNPGSDHDITFSGSFNPSGNAYLSVYGWTTNPLVEYYILENYGSYNPGSGMTHKGTVTSDGSTYDIYEHQQVNQPSIVGTATFNQYWSIRQNKRSSGTVTTANHFKAWASLGMNLGTHNYQIVSTEGYESSGTSTITVSSGGSSSGGSGGSSSTTSSGSSPTGGSGSCSALWGQCGGIGWSGPTCCSSGTCQVSNSYYSQCL

SEQ ID NO: 27 - DNA, 1053 bp, Fusarium verticillioides
atgcagctcaagtttctgtcttcagcattgttgctgtctttgaccggcaattgcgctgcgcaagacactaatgatatccctcctctgatcaccgacctctggtctgcggatccctcggctcatgttttcgagggcaaactctgggtttacccatctcacgacatcgaagccaatgtcgtcaacggcaccggaggcgctcagtacgccatgagagattatcacacctattccatgaagaccatctatggaaaagatcccgttatcgaccatggcgtcgctctgtcagtcgatgatgtcccatgggccaagcagcaaatgtgggctcctgacgcagcttacaagaacggcaaatattatctctacttccccgccaaggataaagatgagatcttcagaattggagttgctgtctccaacaagcccagcggtcctttcaaggccgacaagagctggatccccggtacttacagtatcgatcctgctagctatgtcgacactaatggcgaggcatacctcatctggggcggtatctggggcggccagcttcaggcctggcaggatcacaagacctttaatgagtcgtggctcggcgacaaagctgctcccaacggcaccaacgccctatctcctcagatcgccaagctaagcaaggacatgcacaagatcaccgagacaccccgcgatctcgtcatcctggcccccgagacaggcaagccccttcaagcagaggacaataagcgacgatttttcgaggggccctgggttcacaagcgcggcaagctgtactacctcatgtactctaccggcgacacgcacttcctcgtctacgcgacttccaagaacatctacggtccttatacctatcagggcaagattctcgaccctgttgatgggtggactacgcatggaagtattgttgagtacaagggacagtggtggttgttctttgcggatgcgcatacttctggaaaggattatctgagacaggttaaggcgaggaagatctggtatgacaaggatggcaagattttgcttactcgtcctaagatttag

SEQ ID NO: 28 - protein, 350 aa, Fusarium verticillioides
MQLKFLSSALLLSLTGNCAAQDTNDIPPLITDLWSADPSAHVFEGKLWVYPSHDIEANVVNGTGGAQYAMRDYHTYSMKTIYGKDPVIDHGVALSVDDVPWAKQQMWAPDAAYKNGKYYLYFPAKDKDEIFRIGVAVSNKPSGPFKADKSWIPGTYSIDPASYVDTNGEAYLIWGGIWGGQLQAWQDHKTFNESWLGDKAAPNGTNALSPQIAKLSKDMHKITETPRDLVILAPETGKPLQAEDNKRRFFEGPWVHKRGKLYYLMYSTGDTHFLVYATSKNIYGPYTYQGKILDPVDGWTTHGSIVEYKGQWWLFFADAHTSGKDYLRQVKARKIWYDKDGKILLTRPKI

SEQ ID NO: 29 - DNA, 1031 bp, Penicillium funiculosum
atgagtcgcagcatccttccgtacgcctctgttttcgccctcctgggcggggctatcgccgaaccgtttttggttctcaatagcgattttcccgatcccagtctcatagagacatccagcggatactatgcattcggtaccaccggaaacggagtcaatgcgcaggttgcttcttcaccagactttaatacctggactttgctttccggcacagatgccctcccgggaccatttccgtcatgggtagcttcgtctccacaaatctgggcgccagatgttttggttaaggtatgttcttatggaataacagttttaggagtaggtcagccaggatattgacaaaattataataggccgatggtacctatgtcatgtacttttcggcatctgctgcgagtgactcgggcaaacactgcgttggtgccgcaactgcgacctcaccggaaggaccttacaccccggtcgatagcgctgttgcctgtccattagaccagggaggagctattgatgccaatggatttattgacaccgacggcactatatacgttgtatacaaaattgatggaaacagtctagacggtgatggaaccacacatcctacccccatcatgcttcaacaaatggaggcagacggaacaaccccaaccggcagcccaatccaactcattgaccgatccgacctcgacggacctttgatcgaggctcctagtttgctcctctccaatggaatctactacctcagtttctcttccaactactacaacactaattactacgacacttcatacgcctatgcctcgtcgattactggtccttggaccaaacaatctgcgccttatgcacccttgttggttactggaaccgagactagcaatgacggcgcattgagcgcccctggtggtgccgatttctccgtcgatggcaccaagatgttgttccacgcaaacctcaatggacaagatatctcgggcggacgcgccttatttgctgcgtcaattactgaggccagcgatgtggttacattgcagtag

SEQ ID NO: 30 - protein, 321 aa, Penicillium funiculosum
MSRSILPYASVFALLGGAIAEPFLVLNSDFPDPSLIETSSGYYAFGTTGNGVNAQVASSPDFNTWTLLSGTDALPGPFPSWVASSPQIWAPDVLVKADGTYVMYFSASAASDSGKHCVGAATATSPEGPYTPVDSAVACPLDQGGAIDANGFIDTDGTIYVVYKIDGNSLDGDGTTHPTPIMLQQMEADGTTPTGSPIQLIDRSDLDGPLIEAPSLLLSNGIYYLSFSSNYYNTNYYDTSYAYASSITGPWTKQSAPYAPLLVTGTETSNDGALSAPGGADFSVDGTKMLFHANLNGQDISGGRALFAASITEASDVVTLQ

SEQ ID NO: 31 - DNA, 2186 bp, Fusarium verticillioides
atggttcgcttcagttcaatcctagcggctgcggcttgcttcgtggctgttgagtcagtcaacatcaaggtcgacagcaagggcggaaacgctactagcggtcaccaatatggcttccttcacgaggttggtattgacacaccactggcgatgattgggatgctaacttggagctaggatatcaacaattccggtgatggtggcatctacgctgagctcatccgcaatcgtgctttccagtacagcaagaaataccctgtttctctatctggctggagacccatcaacgatgctaagctctccctcaaccgtctcgacactcctctctccgacgctctccccgtttccatgaacgtgaagcctggaaagggcaaggccaaggagattggtttcctcaacgagggttactggggaatggatgtcaagaagcaaaagtacactggctctttctgggttaagggcgcttacaagggccactttacagcttctttgcgatctaaccttaccgacgatgtctttggcagcgtcaaggtcaagtccaaggccaacaagaagcagtgggttgagcatgagtttgtgcttactcctaacaagaatgcccctaacagcaacaacacttttgctatcacctacgatcccaaggtgagtaacaatcaaaactgggacgtgatgtatactgacaatttgtagggcgctgatggagctcttgacttcaacctcattagcttgttccctcccacctacaagggccgcaagaacggtcttcgagttgatcttgccgaggctctcgaaggtctccaccccgtaaggtttaccgtctcacgtgtatcgtgaacagtcgctgacttgtagaaaagagcctgctgcgcttccccggtggtaacatgctcgagggcaacaccaacaagacctggtgggactggaaggataccctcggacctctccgcaaccgtcctggtttcgagggtgtctggaactaccagcagacccatggtcttggaatcttggagtacctccagtgggctgaggacatgaaccttgaaatcagtaggttctataaaattcagtgacggttatgtgcatgctaacagatttcagttgtcggtgtctacgctggcctctccctcgacggctccgtcacccccaaggaccaactccagcccctcatcgacgacgcgctcgacgagatcgaattcatccgaggtcccgtcacttcaaagtggggaaagaagcgcgctgagctcggccaccccaagcctttcagactctcctacgttgaagtcggaaacgaggactggctcgctggttatcccactggctggaactcttacaaggagtaccgcttccccatgttcctcgaggctatcaagaaagctcaccccgatctcaccgtcatctcctctggtgcttctattgaccccgttggtaagaaggatgctggtttcgatattcctgctcctggaatcggtgactaccacccttaccgcgagcctgatgttcttgttgaggagttcaacctgtttgataacaataagtatggtcacatcattggtgaggttgcttctacccaccccaacggtggaactggctggagtggtaaccttatgccttacccctggtggatctctggtgttggcgaggccgtcgctctctgcggttatgagcgcaacgccgatcgtattcccggaacattctacgctcctatcctcaagaacgagaaccgttggcagtgggctatcaccatgatccaattcgccgccgactccgccatgaccacccgctccaccagctggtatgtctggtcactcttcgcaggccaccccatgacccatactctccccaccaccgccgacttcgaccccctctactacgtcgctggtaagaacgaggacaagggaactcttatctggaagggtgctgcgtataacaccaccaagggtgctgacgttcccgtgtctctgtccttcaagggtgtcaagcccggtgctcaagctgagcttactcttctgaccaacaaggagaaggatccttttgcgttcaatgatcctcacaagggcaacaatgttgttgatactaagaagactgttctcaaggccgatggaaagggtgctttcaacttcaagcttcctaacctgagcgtcgctgttcttgagaccctcaagaagggaaagccttactctagctag

SEQ ID NO: 32 - protein, 660 aa, Fusarium verticillioides
MVRFSSILAAAACFVAVESVNIKVDSKGGNATSGHQYGFLHEDINNSGDGGIYAELIRNRAFQYSKKYPVSLSGWRPINDAKLSLNRLDTPLSDALPVSMNVKPGKGKAKEIGFLNEGYWGMDVKKQKYTGSFWVKGAYKGHFTASLRSNLTDDVFGSVKVKSKANKKQWVEHEFVLTPNKNAPNSNNTFAITYDPKGADGALDFNLISLFPPTYKGRKNGLRVDLAEALEGLHPSLLRFPGGNMLEGNTNKTWWDWKDTLGPLRNRPGFEGVWNYQQTHGLGILEYLQWAEDMNLEIIVGVYAGLSLDGSVTPKDQLQPLIDDALDEIEFIRGPVTSKWGKKRAELGHPKPFRLSYVEVGNEDWLAGYPTGWNSYKEYRFPMFLEAIKKAHPDLTVISSGASIDPVGKKDAGFDIPAPGIGDYHPYREPDVLVEEFNLFDNNKYGHIIGEVASTHPNGGTGWSGNLMPYPWWISGVGEAVALCGYERNADRIPGTFYAPILKNENRWQWAITMIQFAADSAMTTRSTSWYVWSLFAGHPMTHTLPTTADFDPLYYVAGKNEDKGTLIWKGAAYNTTKGADVPVSLSFKGVKPGAQAELTLLTNKEKDPFAFNDPHKGNNVVDTKKTVLKADGKGAFNFKLPNLSVAVLETLKKGKPYSS

SEQ ID NO: 33 - DNA, 2312 bp, Chaetomium globosum
atggcgcccctttcgcttcgggccctctcgctgctcgcgctcacaggagccgcagccgcggtgaccctatcggtcgcgaactctggcggtaatgatacgtctccgtacatgtatggcatcatgttcgaggacatcaatcagagcggtgacggcgggctgtaagttctgtcgcggcttcccctgacaagcttgcatgatgcttaactaaagtccttaggtacgccgagctgattcgcaaccgagccttccataatagctccctccaggcctggaccgccgtgggggacagcactctcgaggtcgtaacctctgcaccgttatcggatgccctgcctcgctcggtcaaggtcacgagtggaaagggcaaggcgggcttgaagaatgccggctactggggaatggacgtccagaagaccgacaagtatagcggcagcttctactcgtacggcgcctacgacggaaagtttaccctctctctggtgtcggacatcacaaatgagaccctggccaccaccaagatcaagtccaggtcggtggagcatgcctggaccgagcacaagttcgagcttctcccgaccaagagcgcggcgaacagcaacaacagcttcgtgctggagttccgcccctgccaccagacggagctccagttcaacctcatcagcttgttcccgccgacgtataagaacaggcccaacggcatgcgccgagagctcatggagaagctcgcagacctcaagcccagtttccttcggattccaggaggcaacaacctgtaagtgcttccggcgaaactagcagtagttgcctgagagacactaatctcagcgaacaacagcgagggcaactatgctggcaactactggaactggtcaagcacacttggcccgctgaccgaccggcccggtcgtgacggcgtgtggacgtacgccaacacggacggcatcgggctggtcgagtacatgcactgggccgaggacctcgacgtggaggttgtgctggcggtcgccgcaggcctgtacctgaacggcgatgtggtcccggaggaggagctgcacgtcttcgtggaggatgcgctgaacgagctcgagttcctcatgggcgacgtctcgaccccttggggcgcgcgccgcgctaagctcggctaccccaagccgtggaacatcaagttcgtcgaggtcggcaacgaggacaacctgtggggcggcctcgactcgtacaagagctaccggctgaagactttctacgacgccatcaaggcgaagtaccccgacatctccatcttttcgtcgaccgacgagtttgtgtacaaggagtcgggccaggactaccacaagtacacccggccggactactccgtgtcccagttcgacctgtttgacaactgggccgacggccaccccatcatcatcggagagtgagtgaacggcgacccccacctccccctaacgcgggatcgcgagctgatagatcaccccaggtatgcgaccatccagaacaacacgggcaagctcgaggacacggactgggacgcgcccaagaacaagtggtccaactggatcggctccgtcgccgaggccgtcttcatcctcggagccgagcgcaacggcgaccgggtctggggcaccacctttgcgccgatcctccagaacctcaacagctaccaatgggctgtaagtacatacatacataccgcacccccaaccccaacccccccaaagcgcacctccacccacccacccaaacacaccacaactacctagctaacccgccacacaaacaaacagcccgacctaatctccttcaccgccaacccggccgacaccacgcccagcgtctcgtacccgatcatccagctgctcgcctcgcaccgcatcacgcacaccctccccgtcagcagcgccgacgccttcggcccggcctactgggtggccggtcgcggcgccgacgacggctcgtacatcctcaaggcggccgtgtacaacagcacggggggtgcggatgtaccggtgagggtgcagtttgaggcggggggtggtggtggtggtggtggtggtggtggtggtggtggtggtgatgggaaggggaagggtaaagggaagggaggggagggtggtgagggtgtgaagaagggtgaccgcgcgcagttgaccgtgttgacggcgccggaggggccctgggcgcataatacgccggagaataagggggcggtcaagacgacagtgacgacgttgaaggccgggaggggtggggtgtttgagtttagtctgccggatttgtcggtggcggtgttggtggtggagggggagaagtga

SEQ ID NO: 34 - protein, 670 aa, Chaetomium globosum
MAPLSLRALSLLALTGAAAAVTLSVANSGGNDTSPYMYGIMFEDINQSGDGGLYAELIRNRAFHNSSLQAWTAVGDSTLEVVTSAPLSDALPRSVKVTSGKGKAGLKNAGYWGMDVQKTDKYSGSFYSYGAYDGKFTLSLVSDITNETLATTKIKSRSVEHAWTEHKFELLPTKSAANSNNSFVLEFRPCHQTELQFNLISLFPPTYKNRPNGMRRELMEKLADLKPSFLRIPGGNNLEGNYAGNYWNWSSTLGPLTDRPGRDGVWTYANTDGIGLVEYMHWAEDLDVEVVLAVAAGLYLNGDVVPEEELHVFVEDALNELEFLMGDVSTPWGARRAKLGYPKPWNIKFVEVGNEDNLWGGLDSYKSYRLKTFYDAIKAKYPDISIFSSTDEFVYKESGQDYHKYTRPDYSVSQFDLFDNWADGHPIIIGEYATIQNNTGKLEDTDWDAPKNKWSNWIGSVAEAVFILGAERNGDRVWGTTFAPILQNLNSYQWAPDLISFTANPADTTPSVSYPIIQLLASHRITHTLPVSSADAFGPAYWVAGRGADDGSYILKAAVYNSTGGADVPVRVQFEAGGGGGGGGGGGGGGGDGKGKGKGKGGEGGEGVKKGDRAQLTVLTAPEGPWAHNTPENKGAVKTTVTTLKAGRGGVFEFSLPDLSVAVLVVEGEK

SEQ ID NO: 35 - DNA, 1002 bp, Fusarium verticillioides
atgcgtcttctatcgtttcccagccatctcctcgtggccttcctaaccctcaaagaggcttcatccctcgccctcagcaaacgggatagccctgtcctccccggcctctgggcggaccccaacatcgccatcgtcgacaagacatactacatcttccctaccaccgacggtttcgaaggctggggcggcaacgtcttctactggtggaaatcaaaagatctcgtatcatggacaaagagcgacaagccattccttactctcaatggtacgaatggcaacgttccctgggctacaggtaatgcctgggctcctgctttcgctgctcgcggaggcaagtattacttctaccatagtgggaataatccctctgtgagtgatgggcataagagtattggtgcggcggtggctgatcatcctgaggggccgtggaaggcacaggataagccgatgatcaagggaacttctgatgaggagattgtcagcaaccaggctatcgatcccgctgcctttgaagaccctgagactggaaagtggtatatctactggggaaacggtgtccccattgtcgcagagctcaacgacgacatggtctctctcaaagcaggctggcacaaaatcacaggtcttcagaatttccgcgagggtcttttcgtcaactatcgcgatggaacatatcatctgacatactctatcgacgatacgggctcagagaactatcgcgttgggtacgctacggcggataaccccattggaccttggacatatcgtggtgttcttctggagaaggacgaatcgaagggcattcttgctacgggacataactccatcatcaacattcctggaacggatgagtggtatatcgcgtatcatcgcttccatattcccgatggaaatgggtataatagggagactacgattgatagggtacccatcgacaaggatacgggtttgtttggaaaggttacgccgactttgcagagtgttgatcctaggcctttgtag

SEQ ID NO: 36 - protein, 333 aa, Fusarium verticillioides
MRLLSFPSHLLVAFLTLKEASSLALSKRDSPVLPGLWADPNIAIVDKTYYIFPTTDGFEGWGGNVFYWWKSKDLVSWTKSDKPFLTLNGTNGNVPWATGNAWAPAFAARGGKYYFYHSGNNPSVSDGHKSIGAAVADHPEGPWKAQDKPMIKGTSDEEIVSNQAIDPAAFEDPETGKWYIYWGNGVPIVAELNDDMVSLKAGWHKITGLQNFREGLFVNYRDGTYHLTYSIDDTGSENYRVGYATADNPIGPWTYRGVLLEKDESKGILATGHNSIINIPGTDEWYIAYHRFHIPDGNGYNRETTIDRVPIDKDTGLFGKVTPTLQSVDPRPL

SEQ ID NO: 37 - DNA, 1695 bp, Fusarium verticillioides
atgctcttctcgctcgttcttcctacccttgcctttcaagccagcctggcgctcggcgatacatccgttactgtcgacaccagccagaaactccaggtcatcgatggctttggtgtctcagaagcctacggccacgccaaacaattccaaaacctcggtcctggaccacagaaagagggcctcgatcttctcttcaacactacaaccggcgcaggcttatccatcatccgaaacaagatcggctgcgacgcctccaactccatcaccagcaccaacaccgacaacccagataagcaggctgtttaccattttgacggcgatgatgatggtcaggtatggtttagcaaacaggccatgagctatggtgtagatactatctacgctaatgcttggtctgcgcctgtatacatgaagtcagcccagagtatgggccgtctctgcggtacacctggtgtgtcgtgctcctctggagattggagacatcgttacgttgagatgatagctgagtacctctcctactacaagcaggctggcatcccagtgtcgcacgttggattcctcaatgagggtgacggctcggactttatgctctcaactgccgaacaggctgcagatgtcattcctcttctacacagcgctttgcagtccaagggccttggcgatatcaagatgacgtgctgtgataacatcggttggaagtcacagatggactataccgccaagctggctgagcttgaggtggagaagtatctatctgtcatcacatcccacgagtactccagcagccccaaccagcctatgaacactacattgccaacctggatgtccgagggagctgccaatgaccaggcatttgccacagcgtggtacgtcaacggcggttccaacgaaggtttcacatgggcagtcaagatcgcacaaggcatcgtcaatgccgacctctcagcgtatatctactgggagggcgttgagaccaacaacaaggggtctctatctcacgtcatcgacacggacggtaccaagtttaccatatcctcgattctctgggccattgctcactggtcgcgccatattcgccctggtgcgcatagactttcgacttcaggtgttgtgcaagatacgattgttggtgcgtttgagaacgttgatggcagtgtcgtcatggtgctcaccaactctggcactgctgctcagactgtggacctgggtgtttcgggaagtagcttctcaacagctcaggctttcacttcggatgctgaggcgcagatggtcgataccaaggtgactctgtccgacggtcgtgtcaaggttacggtcccggtgcacggtgtcgtcactgtgaagctcacaacagcaaaaagctccaaaccggtctcaactgctgtttctgcgcaatctgcccccactccaactagtgttaagcacaccttgactcaccagaagacttcttcaacaacactctcgaccgccaaggccccaacctccactcagactacctctgtagttgagtcagccaaggcggtgaaataccctgtcccccctgtagcatccaagggatcctcgaagagtgctcccaagaagggtaccaagaagaccactacgaagaagggctcccaccaatcgcacaaggcgcatagtgctactcatcgtcgatgccgccatggaagttaccgtcgtggccactgcaccaactaa

SEQ ID NO: 38 - protein, 537 aa, Fusarium verticillioides
MLFSLVLPTLAFQASLALGDTSVTVDTSQKLQVIDGFGVSEAYGHAKQFQNLGPGPQKEGLDLLFNTTTGAGLSIIRNKIGCDASNSITSTNTDNPDKQAVYHFDGDDDGQSAQSMGRLCGTPGVSCSSGDWRHRYVEMIAEYLSYYKQAGIPVSHVGFLNEGDGSDFMLSTAEQAADVIPLLHSALQSKGLGDIKMTCCDNIGWKSQMDYTAKLAELEVEKYLSVITSHEYSSSPNQPMNTTLPTWMSEGAANDQAFATAWYVNGGSNEGFTWAVKIAQGIVNADLSAYIYWEGVETNNKGSLSHVIDTDGTKFTISSILWAIAHWSRHIRPGAHRLSTSGVVQDTIVGAFENVDGSVVMVLTNSGTAAQTVDLGVSGSSFSTAQAFTSDAEAQMVDTKVTLSDGRVKVTVPVHGVVTVKLTTAKSSKPVSTAVSAQSAPTPTSVKHTLTHQKTSSTTLSTAKAPTSTQTTSVVESAKAVKYPVPPVASKGSSKSAPKKGTKKTTTKKGSHQSHKAHSATHRRCRHGSYRRGHCTN

SEQ ID NO: 39 - DNA, 948 bp, Fusarium verticillioides
atgtggaaactcctcgtcagcggtcttgtcgccgtcgcgtccctcagcggcgtgaacgctgcttatcctaaccctggtcccgtcaccggcgatactcgtgttcacgaccctacggttgtcaagactcccagcggtggatacttgctggctcatactggcgataacgtttcgctcaagacttcttctgatcgaactgcttggaaggatgcaggtgctgttttccccaacggtgcgccttggactacgcagtacaccaagggcgacaagaacctctgggcccctgatatctcctaccacaacggccagtactatctgtactactccgcctcttccttcggtcagcgtacctctgccatttttctcgctaccagcaagaccggtgcatccggctcgtggaccaaccaaggcgtcgtcgtcgagtccaacaacaacaacgactacaatgccattgacggaaatctctttgtcgactctgatggaaaatggtggctctccttcggctctttctggtccggcatcaagctcatccaactcgaccccaagaccggcaagcgcaccggctcaagcatgtactccctcgccaaacgcgacgcctccgtcgaaggcgccgtcgaggctccgttcatcaccaaacgcggaagcacctactacctctgggtgtcgttcgacaagtgttgccagggcgctgctagcacgtaccgtgtcatggttggacggtcgagcagcattactggtccttatgttgacaaggctggtaagcagatgatgtctggtggaggaacggagattatggctagtcacggatctattcatggaccgggacataatgctgttttcactgataacgatgcggacgttcttgtctatcattactacgataacgctggcacagcgctgttgggcatcaacttgctcagatatgacaatggctggcctgttgcttattag

SEQ ID NO: 40 - protein, 315 aa, Fusarium verticillioides
MWKLLVSGLVAVASLSGVNAAYPNPGPVTGDTRVHDPTVVKTPSGGYLLAHTGDNVSLKTSSDRTAWKDAGAVFPNGAPWTTQYTKGDKNLWAPDISYHNGQYYLYYSASSFGQRTSAIFLATSKTGASGSWTNQGVVVESNNNNDYNAIDGNLFVDSDGKWWLSFGSFWSGIKLIQLDPKTGKRTGSSMYSLAKRDASVEGAVEAPFITKRGSTYYLWVSFDKCCQGAASTYRVMVGRSSSITGPYVDKAGKQMMSGGGTEIMASHGSIHGPGHNAVFTDNDADVLVYHYYDNAGTALLGINLLRYDNGWPVAY

SEQ ID NO: 41 - DNA, 1352 bp, Trichoderma reesei
atgaaagcaaacgtcatcttgtgcctcctggcccccctggtcgccgctctccccaccgaaaccatccacctcgaccccgagctcgccgctctccgcgccaacctcaccgagcgaacagccgacctctgggaccgccaagcctctcaaagcatcgaccagctcatcaagagaaaaggcaagctctactttggcaccgccaccgaccgcggcctcctccaacgggaaaagaacgcggccatcatccaggcagacctcggccaggtgacgccggagaacagcatgaagtggcagtcgctcgagaacaaccaaggccagctgaactggggagacgccgactatctcgtcaactttgcccagcaaaacggcaagtcgatacgcggccacactctgatctggcactcgcagctgcctgcgtgggtgaacaatatcaacaacgcggatactctgcggcaagtcatccgcacccatgtctctactgtggttgggcggtacaagggcaagattcgtgcttgggtgagttttgaacaccacatgccccttttcttagtccgctcctcctcctcttggaacttctcacagttatagccgtatacaacattcgacaggaaatttaggatgacaactactgactgacttgtgtgtgtgatggcgataggacgtggtcaatgaaatcttcaacgaggatggaacgctgcgctcttcagtcttttccaggctcctcggcgaggagtttgtctcgattgcctttcgtgctgctcgagatgctgacccttctgcccgtctttacatcaacgactacaatctcgaccgcgccaactatggcaaggtcaacgggttgaagacttacgtctccaagtggatctctcaaggagttcccattgacggtattggtgagccacgacccctaaatgtcccccattagagtctctttctagagccaaggcttgaagccattcagggactgacacgagagccttctctacaggaagccagtcccatctcagcggcggcggaggctctggtacgctgggtgcgctccagcagctggcaacggtacccgtcaccgagctggccattaccgagctggacattcagggggcaccgacgacggattacacccaagttgttcaagcatgcctgagcgtctccaagtgcgtcggcatcaccgtgtggggcatcagtgacaaggtaagttgcttcccctgtctgtgcttatcaactgtaagcagcaacaactgatgctgtctgtctttacctaggactcgtggcgtgccagcaccaaccctcttctgtttgacgcaaacttcaaccccaagccggcatataacagcattgttggcatcttacaatag

SEQ ID NO: 42 - protein, 347 aa, Trichoderma reesei
MKANVILCLLAPLVAALPTETIHLDPELAALRANLTERTADLWDRQASQSIDQLIKRKGKLYFGTATDRGLLQREKNAAIIQADLGQVTPENSMKWQSLENNQGQLNWGDADYLVNFAQQNGKSIRGHTLIWHSQLPAWVNNINNADTLRQVIRTHVSTVVGRYKGKIRAWDVVNEIFNEDGTLRSSVFSRLLGEEFVSIAFRAARDADPSARLYINDYNLDRANYGKVNGLKTYVSKWISQGVPIDGIGSQSHLSGGGGSGTLGALQQLATVPVTELAITELDIQGAPTTDYTQVVQACLSVSKCVGITVWGISDKDSWRASTNPLLFDANFNPKPAYNSIVGILQ

SEQ ID NO: 43 - protein, 222 aa, Trichoderma reesei
MVSFTSLLAASPPSRASCRPAAEVESVAVEKRQTIQPGTGYNNGYFYSYWNDGHGGVTYTNGPGGQFSVNWSNSGNFVGGKGWQPGTKNKVINFSGSYNPNGNSYLSVYGWSRNPLIEYYIVENFGTYNPSTGATKLGEVTSDGSVYDIYRTQRVNQPSIIGTATFYQYWSVRRNHRSSGSVNTANHFNAWAQQGLTLGTMDYQIVAVEGYFSSGSASITVS

SEQ ID NO: 44 - protein, 871 aa, Podospora anserina
MAYRSLVLGAFASTSLAASVVTPRDPVPPGFVAAPYYPAPHGGWVASWEEAYSKAEALVSQMTLAEKTNITSGIGIFMGNTGSAERLGFPRMCLQDSALGVSSADNVTAFPAGITTGATFDKKLIYARGVAIGEEHRGKGTNVYLGPSVGPLGRKPLGGRNWEGFGSDPVLQAKAAALTIKGVQEQGIIATIKHLIGNEQEMYRMYNPFQPGYSANIDDRTLHELYLWPFAESVHAGVGSAMTAYNAVNGSACSQHSYLINGILKDELGFQGFVMSDWLSHISGVDSALAGLDMNMPGDTNIPLFGFSNWHYELSRSVLNGSVPLDRLNDMVTRIVATWYKFGQDRDHPRPNFSSNTRDRDGLLYPAALFSPKGQVNWFVNVQADHYLIAREVAQDAITLLKNNGSFLPLTTSQSLHVFGTAAQVNPDGPNACMNRACNKGTLGMGWGSGVADYPYLDDPISAIRKRVPDVKFFNTDGFPWFHPTPSPDDVAIVFITSDAGENSFTVEGNNGDRNSAKLAAWHNGDELVRKTAEKYNNVIVVAQTVGPLDLESWIDNPRVKGVLFQHLPGQEAGESLANILFGDVSPSGHLPYSITKRANDFPDSIANLRGFAFGQVQDTYSEGLYIDYRWLNKEKIRPRFAFGHGLSYTNFSFDATIESVTPLSLVPPARAPKGSTPVYSTEIPPASEAYWPEGFNRIWRYLYSWLNKNDADNAYAVGIAGVKKYNYPAGYSTAQKPGPAAGGGEGGNPALWDIAFRVPVTVKNTGDTFSGRASVQAYVQYPEGIPYDTPVVQLRDFEKTRVLAPGEEETVTVELTRKDLSVWDTELQNWVVPGVGGKRYTVWIGEASDRLFTACYTDTGVCEGGRVPPV

SEQ ID NO: 45 - protein, 797 aa, Trichoderma reesei
MVNNAALLAALSALLPTALAQNNQTYANYSAQGQPDLYPETLATLTLSFPDCEHGPLKNNLVCDSSAGYVERAQALISLFTLEELILNTQNSGPGVPRLGLPNYQVWNEALHGLDRANFATKGGQFEWATSFPMPILTTAALNRTLIHQIADIISTQARAFSNSGRYGLDVYAPNVNGFRSPLWGRGQETPGEDAFFLSSAYTYEYITGIQGGVDPEHLKVAATVKHFAGYDLENWNNQSRLGFDAIITQQDLSEYYTPQFLAAARYAKSRSLMCAYNSVNGVPSCANSFFLQTLLRESWGFPEWGYVSSDCDAVYNVFNPHDYASNQSSAAASSLRAGTDIDCGQTYPWHLNESFVAGEVSRGEIERSVTRLYANLVRLGYFDKKNQYRSLGWKDVVKTDAWNISYEAAVEGIVLLKNDGTLPLSKKVRSIALIGPWANATTQMQGNYYGPAPYLISPLEAAKKAGYHVNFELGTEIAGNSTTGFAKAIAAAKKSDAIIYLGGIDNTIEQEGADRTDIAWPGNQLDLIKQLSEVGKPLVVLQMGGGQVDSSSLKSNKKVNSLVWGGYPGQSGGVALFDILSGKRAPAGRLVTTQYPAEYVHQFPQNDMNLRPDGKSNPGQTYIWYTGKPVYEFGSGLFYTTFKETLASHPKSLKFNTSSILSAPHPGYTYSEQIPVFTFEANIKNSGKTESPYTAMLFVRTSNAGPAPYPNKWLVGFDRLADIKPGHSSKLSIPIPVSALARVDSHGNRIVYPGKYELALNTDESVKLEFELVGEEVTIENWPLEEQQIKDATPDA

SEQ ID NO: 46 - DNA, 2031 bp, Podospora anserina
atgatccacctcaagccagccctcgcggcgttgttggcgctgtcgacgcaatgtgtggctattgatttgtttgtcaagtcttcgggggggaataagacgactgatatcatgtatggtcttatgcacgaggatatcaacaactccggcgacggcggcatctacgccgagctaatctccaaccgcgcgttccaagggagtgagaagttcccctccaacctcgacaactggagccccgtcggtggcgctacccttacccttcagaagcttgccaagcccctttcctctgcgttgccttactccgtcaatgttgccaaccccaaggagggcaagggcaagggcaaggacaccaaggggaagaaggttggcttggccaatgctgggttttggggtatggatgtcaagaggcagaagtacactggtagcttccacgttactggtgagtacaagggtgactttgaggttagcttgcgcagcgcgattaccggggagacctttggcaagaaggtggtgaagggtgggagtaagaaggggaagtggaccgagaaggagtttgagttggtgcctttcaaggatgcgcccaacagcaacaacacctttgttgtgcagtgggatgccgagggcgcaaaggacggatctttggatctcaacttgatcagcttgttccctccgacattcaagggaaggaagaatgggctgagaattgatcttgcgcagacgatggttgagctcaagccgaccttcttgcgcttccccggtggcaacatgctcgagggtaacaccttggacacttggtggaagtggtacgagaccattggccctctgaaggatcgcccgggcatggctggtgtctgggagtaccagcaaacccttggcttgggtctggtcgagtacatggagtgggccgatgacatgaacttggagcccattgtcggtgtcttcgctggtcttgccctcgatggctcgttcgttcccgaatccgagatgggatgggtcatccaacaggctctcgacgaaatcgagttcctcactggcgatgctaagaccaccaaatggggtgccgtccgcgcgaagcttggtcaccccaagccttggaaggtcaagtgggttgagatcggtaacgaggattggcttgccggacgccctgctggcttcgagtcgtacatcaactaccgcttccccatgatgatgaaggccttcaacgaaaagtaccccgacatcaagatcatcgcctcgccctccatcttcgacaacatgacaatccccgcgggtgctgccggtgatcaccacccgtacctgactcccgatgagttcgttgagcgattcgccaagttcgataacttgagcaaggataacgtgacgctcatcggcgaggctgcgtcgacgcatcctaacggtggtatcgcttgggagggagatctcatgcccttgccttggtggggcggcagtgttgctgaggctatcttcttgatcagcactgagagaaacggtgacaagatcatcggtgctacttacgcgcctggtcttcgcagcttggaccgctggcaatggagcatgacctgggtgcagcatgccgccgacccggccctcaccactcgctcgaccagttggtatgtctggagaatcctcgcccaccacatcatccgtgagacgctcccggtcgatgccccggccggcaagcccaactttgaccctctgttctacgttgccggaaagagcgagagtggcaccggtatcttcaaggctgccgtctacaactcgactgaatcgatcccggtgtcgttgaagtttgatggtctcaacgagggagcggttgccaacttgacggtgcttactgggccggaggatccgtatggatacaacgaccccttcactggtatcaatgttgtcaaggagaagaccaccttcatcaaggccggaaagggcggcaagttcaccttcaccctgccgggcttgagtgttgctgtgttggagacggccgacgcggtcaagggtggcaagggaaagggcaagggcaagggaaagggtaactga

SEQ ID NO: 47 - DNA, 2031 bp, Podospora anserina
atgatccacctcaagcccgccctcgccgccctcctcgccctcagcacccaatgcgtcgccatcgacctcttcgtcaagagcagcggcggcaacaagaccaccgacatcatgtacggcctcatgcacgaggacatcaacaacagcggcgacggcggcatctacgccgagctgatcagcaaccgcgccttccagggcagcgagaagttccccagcaacctcgacaactggtcccccgtcggcggcgccaccctcaccctccagaagctcgccaagcccctgtcctctgccctcccctactccgtcaacgtcgccaaccccaaggagggtaagggtaagggcaaggacaccaagggcaagaaggtcggcctcgccaacgccggcttttggggcatggacgtcaagcgccagaaatacaccggcagcttccacgtcaccggcgagtacaagggcgacttcgaggtcagcctccgcagcgccattaccggcgagaccttcggcaagaaggtcgtcaagggcggcagcaagaagggcaagtggaccgagaaggagttcgagctggtccccttcaaggacgcccccaacagcaacaacaccttcgtcgtccagtgggacgccgagggcgccaaggacggcagcctcgacctcaacctcatcagcctcttcccgcccaccttcaagggccgcaagaacggcctccgcatcgacctcgcccagaccatggtcgagctgaagcccaccttcctccgctttcccggcggcaacatgctcgagggcaacaccctcgacacctggtggaagtggtacgagaccatcggccccctgaaggaccgccctggcatggccggcgtctgggagtaccagcagacgctgggcctcggcctggtcgagtacatggagtgggccgacgacatgaacctcgagcccatcgtcggcgtctttgctggcctggccctggatggcagctttgtccccgagagcgagatgggctgggtcatccagcaggctctcgatgagatcgagttcctcaccggcgacgccaagaccaccaagtggggcgccgtccgcgccaagctcggccaccctaagccctggaaggtcaaatgggtcgagatcggcaacgaggactggctcgccggccgacctgccggcttcgagagctacatcaactaccgcttccccatgatgatgaaggccttcaacgagaaataccccgacatcaagatcattgccagcccctccatcttcgacaacatgaccattccagccggtgctgccggtgaccaccacccctacctcacccccgacgaatttgtcgagcgcttcgccaagttcgacaacctcagcaaggacaacgtcaccctcattggcgaggccgccagcacccaccccaacggcggcattgcctgggagggcgacctcatgcccctgccctggtggggcggcagcgtcgccgaggccatcttcctcatcagcaccgagcgcaacggcgacaagatcatcggcgccacctacgcccctggcctccgatctctcgaccgctggcagtggagcatgacctgggtccagcacgccgccgaccctgccctcaccacccgcagcaccagctggtacgtctggcgcatcctcgcccaccacatcattcgcgagaccctccccgtcgacgcccccgccggcaagcccaacttcgaccccctcttctacgtcgctggcaagtcggagagcggcaccggcatcttcaaggccgccgtctacaacagcaccgagagcatccccgtcagcctcaagttcgacggcctcaacgagggcgccgtcgccaacctcaccgtcctcaccggccccgaggacccctacggctacaacgaccccttcaccggcatcaacgtcgtcaaggaaaagaccaccttcatcaaggccggcaagggcggcaagttcacctttaccctccccggcctctctgtcgccgtcctcgagaccgccgacgccgtgaagggtggcaagggaaagggaaagggcaagggtaagggtaactaa

SEQ ID NO: 48 - DNA, 1020 bp, Gibberella zeae
atgtatcggaagttggccgtcatctcggccttcttggccacagctcgtgctaccaacgacgactgtcctctcatcactagtagatggactgcggatccttcggctcatgtctttaacgacaccttgtggctctacccgtctcatgacatcgatgctggatttgagaatgatcctgatggaggccagtacgccatgagagattaccatgtctactctatcgacaagatctacggttccctgccggtcgatcacggtacggccctgtcagtggaggatgtcccctgggcctctcgacagatgtgggctcctgacgctgcccacaagaacggcaaatactacctatacttccctgccaaagacaaggatgatatcttcagaatcggcgttgctgtctcaccaacccccggcggaccattcgtccccgacaagagttggatccctcacactttcagcatcgaccccgccagtttcgtcgatgatgatgacagagcctacttggcatggggtggtatcatgggtggccagcttcaacgatggcaggataagaacaagtacaacgaatctggcactgagccaggaaacggcaccgctgccttgagccctcagattgccaagctgagcaaggacatgcacactctggcagagaagcctcgcgacatgctcattcttgaccccaagactggcaagccgctcctttctgaggatgaagaccgacgcttcttcgaaggaccctggattcacaagcgcaacaagatttactacctcacctactctactggcacaacccactatcttgtctatgcgacttcaaagaccccctatggtccttacacctaccagggcagaattctggagccagttgatggctggactactcactctagtatcgtcaagtaccagggtcagtggtggctattttatcacgatgccaagacatctggcaaggactatcttcgccaggtaaaggctaagaagatttggtacgatagcaaaggaaagatcttgacaaagaagccttga

SEQ ID NO: 49 - DNA, 1038 bp, Fusarium oxysporum
atgtatcggaagttggccgtcatctcggccttcttggccacagctcgtgctcaagacactaatgacattcctcccctgatcaccgacctctggtccgcagatccctcggctcatgttttcgaaggcaagctctgggtttacccatctcacgacatcgaagccaatgttgtcaacggcacaggaggcgctcaatacgccatgagggattaccatacctactccatgaagagcatctatggtaaagatcccgttgtcgaccacggcgtcgctctctcagtcgatgacgttccctgggcgaagcagcaaatgtgggctcctgacgcagctcataagaacggcaaatattatctgtacttccccgccaaggacaaggatgagatcttcagaattggagttgctgtctccaacaagcccagcggtcctttcaaggccgacaagagctggatccctggcacgtacagtatcgatcctgctagctacgtcgacactgataacgaggcctacctcatctggggcggtatctggggcggccagctccaagcctggcaggataaaaagaactttaacgagtcgtggattggagacaaggctgctcctaacggcaccaatgccctatctcctcagatcgccaagctaagcaaggacatgcacaagatcaccgaaacaccccgcgatctcgtcattctcgcccccgagacaggcaagcctcttcaggctgaggacaacaagcgacgattcttcgagggcccttggatccacaagcgcggcaagctttactacctcatgtactccaccggtgatacccacttccttgtctacgctacttccaagaacatctacggtccttatacctaccggggcaagattcttgatcctgttgatgggtggactactcatggaagtattgttgagtataagggacagtggtggcttttctttgctgatgcgcatacgtctggtaaggattaccttcgacaggtgaaggcgaggaagatctggtatgacaagaacggcaagatcttgcttcaccgtccttag

SEQ ID NO: 50 - DNA, 1920 bp, Penicillium funiculosum
atgtaccggaagctcgccgtgatcagcgccttcctggcgactgctcgcgccatcaccatcaacgtcagccagagcggcggcaacaagaccagcccgctccagtacggcctcatgttcgaggacatcaaccacggcggcgacggcggcctctacgccgagctggtccggaaccgggccttccagggcagcaccgtctacccggccaacctcgacggctacgactcggtgaacggcgcgattctcgcgctccagaacctcaccaacccgctcagcccgagcatgccctcgtcgctgaacgtcgccaagggctcgaacaacggcagcatcggcttcgccaacgaggggtggtggggcatcgaggtcaagccgcagcggtacgccggcagcttctacgtccagggcgactaccagggcgacttcgacatcagcctccagagcaagctcacccaggaggtcttcgcgacggcgaaggtccggtcgagcggcaagcacgaggactgggtccagtacaagtacgagctggtcccgaagaaggccgccagcaacaccaacaacaccctcaccatcaccttcgacagcaagggcctcaaggacggcagcctcaacttcaacctcatcagcctcttcccgccgacctacaacaaccggccgaacggcctccggatcgacctcgtcgaggccatggcggagctggagggcaagttcctccgcttccccggcggctcggacgtggagggcgtccaggccccgtactggtacaagtggaacgagaccgtcggcgacctcaaggaccgctactcgcgcccgagcgcctggacctacgaggagagcaacggcatcggcctcatcgagtacatgaactggtgcgacgacatgggcctcgagccgatcctcgccgtctgggacggccactacctcagcaacgaggtcatcagcgagaacgacctccagccgtacatcgacgacaccctcaaccagctcgagttcctcatgggcgccccggacactccctacgggtcttggagggctagcctcggctacccgaagccgtggaccatcaactacgtcgagatcggcaacgaggacaacctctacggcggcctcgagacctacatcgcctaccggttccaggcctactacgacgccatcaccgccaagtacccgcacatgaccgtcatggagagcctcaccgagatgcccggccccgctgccgcggcgtcggactaccaccagtactcgacgcccgacggcttcgtcagccagttcaactacttcgaccagatgccggtcaccaaccgcacgctgaacggcgagatcgccaccgtctaccccaacaacccgagcaactcggtggcgtggggcagcccgttcccgctctacccgtggtggatcgggtccgtggctgaggccgtcttcctcatcggcgaggagcggaacagcccgaagatcatcggcgccagctacgcccccatgttccgcaacattaacaactggcagtggagcccgaccctgatcgccttcgacgccgacagcagccggacgtcgcgctctacttcctggcacgtcatcaagctcctcagcaccaacaagatcacccagaacctgcccacgacgtggtctgggggggacatcggcccgctctactgggtcgccggccggaacgacaacaccggcagcaacatcttcaaggccgccgtctacaacagcaccagcgacgtcccggtcaccgtccagttcgccggctgcaacgccaagagcgccaacctcaccatcctctcgtcggacgaccccaacgccagcaactacccgggcggccccgaggtcgtcaagaccgagatccagagcgtcaccgccaacgcccacggcgccttcgagttcagcctcccgaacctgtcggtggctgtgctgaagacggagtag

SEQ ID NO: 51 - DNA, 1044 bp, Trichoderma reesei
atgatccagaagctttccaaccttcttctcaccgcactagcggtggcaaccggtgttgttggacacggacacatcaacaacattgtcgtcaacggagtgtactaccagggatatgatcctacatcgttcccatatgaatctgacccgcccatagtggtgggctggacggctgccgatcttgacaacggcttcgtctcacccgacgcatatcagagcccggacatcatctgccacaagaatgccaccaacgccaaaggacacgcgtccgtcaaggccggagacactattcccctccagtgggtgccagttccttggccgcacccaggccccatcgtcgactacctggccaactgcaacggcgactgcgagaccgtggacaagacgtcccttgagttcttcaagattgacggcgtcggtctcatcagcggcggagatccgggcaactgggcctcggacgtgttgattgccaacaacaacacctgggttgtcaagatccccgaggatctcgccccgggcaactacgtgcttcgccacgagatcatcgccttgcacagcgccgggcaggcggacggcgctcagaactaccctcagtgcttcaacctcgccgtcccaggctccggatctctgcagccgagcggcgtcaagggaaccgcgctctaccactccgatgaccccggtgtcctcatcaacatctacaccagccctcttgcgtacaccattcctggaccttccgtggtatcaggcctccccacgagtgtcgcccagggcagctccgccgcgacggccactgccagcgccactgttcctggcggtagcggaccgggaaacccgaccagtaagactacgacgacggcgaggacgacacaggcctcctctagcagggccagctctactcctcctgctactacgtcggcacctggtggaggcccaacccagactttgtacggccagtgtggtggcagcggctacagtggtcctactcgatgcgcgccgccggccacttgctctaccttgaacccatactacgcccagtgccttaactag

SEQ ID NO: 52 - protein, 344 aa, Trichoderma reesei
MIQKLSNLLVTALAVATGVVGHGHINDIVINGVWYQAYDPTTFPYESNPPIVVGWTAADLDNGFVSPDAYQNPDIICHKNATNAKGHASVKAGDTILFQWVPVPWPHPGPIVDYLANCNGDCETVDKTTLEFFKIDGVGLLSGGDPGTWASDVLISNNNTWVVKIPDNLAPGNYVLRHEIIALHSAGQANGAQNYPQCFNIAVSGSGSLQPSGVLGTDLYHATDPGVLINIYTSPLNYIIPGPTVVSGLPTSVAQGSSAATATASATVPGGGSGPTSRTTTTARTTQASSRPSSTPPATTSAPAGGPTQTLYGQCGGSGYSGPTRCAPPATCSTLNPYYAQCLN

SEQ ID NO: 53 - DNA, 2260 bp, Podospora anserina
atggctcttcaaaccttcttcctgctggcggcagccatgctggccaacgcagagacaacaggcgaaaaggtctctcggcaagcaccgtctggcgctcaagcatgggccgccgcccactcccaggctgccgccactctggccagaatgtcacagcaagacaagatcaacatggtcacgggcattggctgggacagagggccttgcgtgggaaacacagctgccatcagctccatcaactatcctcaaatctgtcttcaggatggaccattgggcattcgcttcggcactggtaccaccgccttcacacctggcgtccaagctgcttcgacatgggacgttgatctgatccggcagcgcggtgcttacctgggcgccgaagccaagggctgcggcattcacatccttttggggcccgttgccggtgccctgggcaagattccccacggcggtcgcaactgggagggatttggcgccgacccctaccttgccggtattgccatgaaggagaccatcgagggtattcagtcagcaggcgtccaggccaacgccaagcactacattgcaaacgaacaagagctcaaccgcgagaccatgagcagcaatgtggatgaccgcactcagcacgagctctacctctggccctttgccgacgccgtgcacgccaacgtcgccagcgtcatgtgcagttacaacaagctcaatggcacgtgggcttgcgagaatgacaaggctctgaatcagatcttgaagaaggagctcggattccagggctacgttctcagcgactggaatgctcagcacagcactgctctgtctgctaacagtggtctggacatgactatgcccggtaccgatttcaacggccgcaatgtctactggggccctcaactgaacaacgctgtcaacgccggccaggttcagagatccagactagacgacatgtgcaagagaatcttggctggctggtacttgctcggtcagaaccagggctatcccgccatcaacatcagggccaacgttcagggcaaccataaggagaacgtacgtgctgttgccagagacggcatcgtcttgctgaagaacgatggaattctgccgctttccaagccgagaaagattgctgtcgtgggctcccactccgtcaacaatccccagggaatcaacgcctgtgttgacaagggctgcaatgttggcacccttggcatgggctggggttcaggcagcgtcaactacccctatctcgtgtccccgtacgatgctctccggactcgtgctcaggccgatggcacacaaatcagcctccacaacactgacagcaccaacggtgtgtcaaacgttgtgtctgacgctgatgctgttgttgttgtcatcactgccgattctggtgaagggtacatcactgtcgagggccacgctggcgaccgcagccaccttgacccgtggcacaatggcaaccaacttgttcaggctgccgcggctgccaacaagaacgtcatcgttgttgtgcacagtgttggccagatcaccctggagactatcctcaacaccaatggagtccgcgcgattgtgtgggctggtcttccgggccaagagaatggcaacgctcttgttgatgttctctacggcttggtttcgccatctggaaagcttccctacaccattggcaagagggagtcggactatggcacagccgttgttcgtggggatgataacttcagggagggcctttttgttgactaccgtcactttgacaatgccaggatcgagccgcgctatgagtttggctttggtctttgtaagttccagcggcggagttgggtttgatttcaagctttcctaacctgataaaacagcttacaccaatttcaccttctccgacatcaagattacttccaatgtcaagccggggcccgctactggccagaccattcccggcggacctgccgacctgtgggaggacgttgcgacagtcactgcaaccatcaccaactcgggtgctgtcgagggcgctgaggttgcccagctttacatcggcctgccgtcctcggctcctgcctctcccccgaagcagctgcgtggattttccaagctgaagctggccccgggtgccagcggcactgccacattcaacctcagacgcagagatctcagctattgggatacccgcctccagaactgggtcgtgcccagcggcaactttgtcgtcagcgtcggcgccagctcgagagatatccgcttgacgggcaccatcacggcgtag

SEQ ID NO: 54 - protein, 733 aa, Podospora anserina
MALQTFFLLAAAMLANAETTGEKVSRQAPSGAQAWAAAHSQAAATLARMSQQDKINMVTGIGWDRGPCVGNTAAISSINYPQICLQDGPLGIRFGTGTTAFTPGVQAASTWDVDLIRQRGAYLGAEAKGCGIHILLGPVAGALGKIPHGGRNWEGFGADPYLAGIAMKETIEGIQSAGVQANAKHYIANEQELNRETMSSNVDDRTQHELYLWPFADAVHANVASVMCSYNKLNGTWACENDKALNQILKKELGFQGYVLSDWNAQHSTALSANSGLDMTMPGTDFNGRNVYWGPQLNNAVNAGQVQRSRLDDMCKRILAGWYLLGQNQGYPAINIRANVQGNHKENVRAVARDGIVLLKNDGILPLSKPRKIAVVGSHSVNNPQGINACVDKGCNVGTLGMGWGSGSVNYPYLVSPYDALRTRAQADGTQISLHNTDSTNGVSNVVSDADAVVVVITADSGEGYITVEGHAGDRSHLDPWHNGNQLVQAAAAANKNVIVVVHSVGQITLETILNTNGVRAIVWAGLPGQENGNALVDVLYGLVSPSGKLPYTIGKRESDYGTAVVRGDDNFREGLFVDYRHFDNARIEPRYEFGFGLSYTNFTFSDIKITSNVKPGPATGQTIPGGPADLWEDVATVTATITNSGAVEGAEVAQLYIGLPSSAPASPPKQLRGFSKLKLAPGASGTATFNLRRRDLSYWDTRLQNWVVPSGNFVVSVGASSRDIRLTGTITA

SEQ ID NO: 55 - DNA, 2551 bp, Fusarium verticillioides
atgtttccttcttccatatcttgtttggcggccctgagtctgatgagccagggtctactagctcagagccaaccggaaaatgtcatcaccgatgatacctacttctacggtcaatcgccaccagtgtatcctacacgtaagcactctctctgatttcccaacgaaagcaatactgatctcttgaccagcggaacaggtagacaccggctcatgggctgccgctgtagccaaagccaagaacttggtgtcccagttgactcttgaagagaaagtcaacttgactacaggaggccagacgaccaccggctgctctggcttcatccctggcattccccgtgtaggctttccaggactgtgtttagcagacgctggcaacggtgtccgcaacacagattatgtgagctcgtttccctccgggattcatgtcggtgcaagctggaatccggagttgacctacagccggagctactacatgggtgctgaggccaaagccaagggcgttaacatccttctcggtccagtatttggacctttgggccgagtagttgaaggtggacgcaactgggaggggttttccaatgatccctacctggcgggtaaattagggcatgaagctgtcgccggtatccaagacgccggagttgttgcatgcggaaaacatttccttgctcaagagcaggagacccatagacttgcggcgtctgtcactggggctgatgcaatctcatcaaatctcgatgacaagacactccatgaattatatctctggtaagcacatcatatcttggctgagtagatgaaccttactaacacccgaactgggcttttcgctgatgcagtccacgccggacttgccagtgtgatgtgcagctacaacagagcaaacaattcacacgcctgccaaaactcgaagcttctcaatggccttctcaagggcgagttaggattccagggttttgtcgtctcggactggggcgcacagcaatctggtatggcttcagcattggctggcctggatgttgtcatgcccagctcgatcttgtggggtgccaaccttacccttggtgtgaacaacggaactattcccgagtcacaggttgacaatatggttacacggtacgcgaagtctcagccttacttctcaattcttttgaactgacaatcgtgtaggctccttgcaacttggtatcagttgaaccaggaccaagacaccgaagccccaggtcacggactcgctgccaagctttgggagcctcacccagtagtcgacgctcgcaacgcaagctccaagcctactatctgggacggtgcagtcgagggccatgttcttgttaagaacaccaacaacgcactgccattcaagcccaacatgaaactcgtttctttgttcggatactctcacaaagctcctgataagaacatcccagaccccgcccaaggcatgttctccgcttggtctatcggtgcccaatccgccaacatcactgagctgaacctcggctttctcggaaatttgagtctcacatactccgccatcgcgcccaacggaaccatcatctcgggtggaggctcgggtgccagcgcttggactctgttcagctcacccttcgatgcattcgtttctcgggcgaagaaagagggtactgcgcttttctgggattttgagagctgggatccttatgtgaaccctacatctgaagcttgcatcgttgctggtaatgcatgggctagcgaaggctgggatagacctgcaacctatgatgcctatactgatgagctcatcaataacgtcgctgacaagtgcgctaacactattgttgttcttcacaatgctggaacacgacttgtggatggcttctttggtcaccccaacgtcaccgctattatctacgctcatctcccaggtcaggatagtggagatgctctggtatctttgctctatggcgatgagaacccatctggtcgcctcccttacaccgttgcccgcaacgagacggattatggtcacctgctgaagccagacttgactctcgcccccaaccagtaccaacactttccccagtccgacttctccgagggtattttcattgactaccgacatttcgatgctaagaacatcacgcctcgcttcgagtttggtttcggcttgagctacacaacctttgagtacgctagtctccagatctcaaagtcccaggcccagacaccggaatacccagctggtgctcttaccgagggaggccgttcagatttgtgggacgtcgttgctactgtcacagcaagcgtcaggaacactgggtctgtcgacggcaaggaggttgcacagctatacgttggtgttccaggtggtcctatgagacagctacgtggctttacgaaaccagctattaaggctggagagacggctacagtgacctttgagcttactcgccgcgacttgagtgtctgggatgttaatgcgcaggagtggcaacttcagcaaggcaactatgctatctacgttggccgaagtagtcgagatttgcctctgcaaagtaccttgagcatctag

SEQ ID NO: 56 - protein, 780 aa, Fusarium verticillioides
MFPSSISCLAALSLMSQGLLAQSQPENVITDDTYFYGQSPPVYPTHTGSWAAAVAKAKNLVSQLTLEEKVNLTTGGQTTTGCSGFIPGIPRVGFPGLCLADAGNGVRNTDYVSSFPSGIHVGASWNPELTYSRSYYMGAEAKAKGVNILLGPVFGPLGRVVEGGRNWEGFSNDPYLAGKLGHEAVAGIQDAGVVACGKHFLAQEQETHRLAASVTGADAISSNLDDKTLHELYLCVMCSYNRANNSHACQNSKLLNGLLKGELGFQGFVVSDWGAQQSGMASALAGLDVVMPSSILWGANLTLGVNNGTIPESQVDNMVTRLLATWYQLNQDQDTEAPGHGLAAKLWEPHPVVDARNASSKPTIWDGAVEGHVLVKNTNNALPFKPNMKLVSLFGYSHKAPDKNIPDPAQGMFSAWSIGAQSANITELNLGFLGNLSLTYSAIAPNGTIISGGGSGASAWTLFSSPFDAFVSRAKKEGTALFWDFESWDPYVNPTSEACIVAGNAWASEGWDRPATYDAYTDELINNVADKCANTIVVLHNAGTRLVDGFFGHPNVTAIIYAHLPGQDSGDALVSLLYGDENPSGRLPYTVARNETDYGHLLKPDLTLAPNQYQHFPQSDFSEGIFIDYRHFDAKNITPRFEFGFGLSYTTFEYASLQISKSQAQTPEYPAGALTEGGRSDLWDVVATVTASVRNTGSVDGKEVAQLYVGVPGGPMRQLRGFTKPAIKAGETATVTFELTRRDLSVWDVNAQEWQLQQGNYAIYVGRSSRDLPLQSTLSI

SEQ ID NO: 57 - DNA, 2487 bp, Fusarium verticillioides
atggctagcattcgatctgtgttggtctcgggtcttttggccgcgggtgtcaatgcccaagcctacgatgcgagtgatcgcgctgaagatgctttcagctgggtccagcccaagaacaccactattcttggacagtacggccattcgcctcattaccctgccagtatgttcaccaactacaccaagtgacactgaggctgtactgacattctagacaatgctactggcaagggctgggaagatgccttcgccaaggctcaaaactttgtctcccaactaaccctcgaggaaaaggccgacatggtcacaggaactccaggtccttgcgtcggcaacatcgtcgccattccccgtctcaacttcaacggtctctgtcttcacgacggccccctcgccatccgagtagcagactacgccagtgttttccccgctggtgtatcagccgcttcatcgtgggacaaggacctcctctaccagcgcggtctcgccatgggtcaagagttcaaggccaagggtgctcacatcctcctcggccccgtcgccggtcctcttggccgctcggcatactctggtcgtaactgggagggtttctcgccggacccttacctcactggtattgcgatggaggagactatcatgggacatcaagatgctggtgttcaggctactgcgaagcactttatcggtaatgagcaggaggtcatgcgaaaccctacttttgtcaaggatgggtatattggtgaggttgacaaggaggctctttcgtctaacatggatgatcgaaccatgcacgagctttacctctggccctttgccaatgctgttcatgccaaggcttccagcatgatgtgctcgtaccagcgtctcaacggctcctacgcctgccagaactcaaaggtcctcaacggaattctgcgtgatgagcttggtttccagggctacgtcatgtcagattggggtgccacccacgccggtgttgctgccatcaacagcggtctcgacatggacatgcccggtggtatcggtgcctacggaacatactttaccaagtccttcttcggcggcaacctcacccgcgccgtcaccaacggcaccctcgacgagacccgcgtcaacgacatgatcacccgcatcatgactccctacttctggctcggccaggacaaggactatccctccgtcgacccctccagcggtgatctcaacaccttcagccccaagagctcctggttccgcgagttcaacctcaccggcgagcgcagccgtgacgtccgcggtaaccacggcgacttgatccgcaagcacggcgccgagtctaccgtccttctcaagaacgagaagaacgcccttcccctcaagaagcccaagtccatcgctgtctttggcaacgatgctggtgatatcactgagggtttctacaaccagaatgactacgaatttggcactcttgttgctggtggtggctctggaactggtcgtttgacataccttgtttcgcctctagccgccatcaatgctcgtgctaagcaggacggtactcttgttcagcagtggatgaacaacactcttattgctaccaccaacgtcactgatctctggatccctgctactcccgatgtctgcctcgttttcttgaagacttgggctgaggaggctgctgatcgtgagcacctctccgttgactgggacggtaatgatgttgttgagtctgttgccaagtactgcaataacactgtcgtcgtcactcactcttctggtatcaacactcttccttgggctgaccaccccaacgtcaccgctattctcgctgcccacttccccggtcaggagtctggcaactccctcgttgacctcctctacggcgatgtcaacccctctggtcgtcttccctacaccatcgccttcaacggcaccgactacaacgctccccccaccactgccgtcaacaccaccggcaaggaggactggcagtcttggttcgacgagaagctcgagattgactaccgctacttcgacgcgcacaacatctccgtccgctacgaattcggcttcggtctctcctactccaccttcgaaatctccgacatctccgctgagccactcgcatccgacattacctcccagcccgaggatctccccgtgcagcccggcggcaaccccgccctctgggagaccgtctacaacgtgaccgtctccgtctccaacacgggcaaggtcgacggcgccactgtcccccagctatacgtgacattccccgacagcgcgcctgccggtacaccacccaagcagctccgtgggttcgacaaggtcttccttgaggctggcgagagcaagagtgtcagctttgagctgatgcgccgtgatctgagctactgggatatcatttctcagaagtggctcatccctgagggagagtttactattcgtgttggattcagcagtcgggacttgaaggaggagacaaaggttactgttgttgaggcgtaa

SEQ ID NO: 58 - protein, 811 aa, Fusarium verticillioides
MASIRSVLVSGLLAAGVNAQAYDASDRAEDAFSWVQPKNTTILGQYGHSPHYPANNATGKGWEDAFAKAQNFVSQLTLEEKADMVTGTPGPCVGNIVAIPRLNFNGLCLHDGPLAIRVADYASVFPAGVSAASSWDKDLLYQRGLAMGQEFKAKGAHILLGPVAGPLGRSAYSGRNWEGFSPDPYLTGIAMEETIMGHQDAGVQATAKHFIGNEQEVMRNPTFVKDGYIGEVDKEALSSNMDDRTMHELYLWPFANAVHAKASSMMCSYQRLNGSYACQNSKVLNGILRDELGFQGYVMSDWGATHAGVAAINSGLDMDMPGGIGAYGTYFTKSFFGGNLTRAVTNGTLDETRVNDMITRIMTPYFWLGQDKDYPSVDPSSGDLNTFSPKSSWFREFNLTGERSRDVRGNHGDLIRKHGAESTVLLKNEKNALPLKKPKSIAVFGNDAGDITEGFYNQNDYEFGTLVAGGGSGTGRLTYLVSPLAAINARAKQDGTLVQQWMNNTLIATTNVTDLWIPATPDVCLVFLKTWAEEAADREHLSVDWDGNDVVESVAKYCNNTVVVTHSSGINTLPWADHPNVTAILAAHFPGQESGNSLVDLLYGDVNPSGRLPYTIAFNGTDYNAPPTTAVNTTGKEDWQSWFDEKLEIDYRYFDAHNISVRYEFGFGLSYSTFEISDISAEPLASDITSQPEDLPVQPGGNPALWETVYNVTVSVSNTGKVDGATVPQLYVTFPDSAPAGTPPKQLRGFDKVFLEAGESKSVSFELMRRDLSYWDIISQKWLIPEGEFTIRVGFSSRDLKEETKVTVVEA

SEQ ID NO: 59 - DNA, 3269 bp, Fusarium verticillioides
atgaagctgaattgggtcgccgcagccctgtctataggtgctgctggcactgacagcgcagttgctcttgcttctgcagttccagacactttggctggtgtaaaggtcagttttttttcaccatttcctcgtctaatctcagccttgttgccatatcgcccttgttcgctcggacgccacgcaccagatcgcgatcatttcctcccttgcagccttggttcctcttacgatcttccctccgcaattatcagcgcccttagtctacacaaaaacccccgagacagtctttcattgagtttgtcgacatcaagttgcttctcaactgtgcatttgcgtggctgtctacttctgcctctagacaaccaaatctgggcgcaattgaccgctcaaaccttgttcaaataaccttttttattcgagacgcacatttataaatatgcgcctttcaataataccgactttatgcgcggcggctgctgtggcggttgatcagaaagctgacgctcaaaaggttgtcacgagagatacactcgcatactcgccgcctcattatccttcaccatggatggaccctaatgctgttggctgggaggaagcttacgccaaagccaagagctttgtgtcccaactcactctcatggaaaaggtcaacttgaccactggtgttgggtaagcagctccttgcaaacagggtatctcaatcccctcagctaacaacttctcagatggcaaggcgaacgctgtgtaggaaacgtgggatcaattcctcgtctcggtatgcgaggtctctgtctccaggatggtcctcttggaattcgtctgtccgactacaacagcgcttttcccgctggcaccacagctggtgcttcttggagcaagtctctctggtatgagagaggtctcctgatgggcactgagttcaaggagaagggtatcgatatcgctcttggtcctgctactggacctcttggtcgcactgctgctggtggacgaaactgggaaggcttcaccgttgatccttatatggctggccacgccatggccgaggccgtcaagggtattcaagacgcaggtgtcattgcttgtgctaagcattacatcgcaaacgagcagggtaagccacttggacgatttgaggaattgacagagaactgaccctcttgtagagcacttccgacagagtggcgaggtccagtcccgcaagtacaacatctccgagtctctctcctccaacctggatgacaagactatgcacgagctctacgcctggcccttcgctgacgccgtccgcgccggcgtcggttccgtcatgtgctcgtacaaccagatcaacaactcgtacggttgccagaactccaagctcctcaacggtatcctcaaggacgagatgggcttccagggtttcgtcatgagcgattgggcggcccagcataccggtgccgcttctgccgtcgctggtctcgatatgagcatgcctggtgacactgccttcgacagcggatacagcttctggggcggaaacttgactctggctgtcatcaacggaactgttcccgcctggcgagttgatgacatggctctgcgaatcatgtctgccttcttcaaggttggaaagacgatagaggatcttcccgacatcaacttctcctcctggacccgcgacaccttcggcttcgtgcatacatttgctcaagagaaccgcgagcaggtcaactttggagtcaacgtccagcacgaccacaagagccacatccgtgaggccgctgccaagggaagcgtcgtgctcaagaacaccgggtcccttcccctcaagaacccaaagttcctcgctgtcattggtgaggacgccggtcccaaccctgctggacccaatggttgtggtgaccgtggttgcgataatggtaccctggctatggcttggggctcgggaacttcccaattcccttacttgatcacccccgatcaagggctctctaatcgagctactcaagacggaactcgatatgagagcatcttgaccaacaacgaatgggcttcagtacaagctcttgtcagccagcctaacgtgaccgctatcgttttcgccaatgccgactctggtgagggatacattgaagtcgacggaaactttggtgatcgcaagaacctcaccctctggcagcagggagacgagctcatcaagaacgtgtcgtccatatgccccaacaccattgtagttctgcacaccgtcggccctgtcctactcgccgactacgagaagaaccccaacatcactgccatcgtctgggctggtcttcccggccaagagtcaggcaatgccatcgctgatctcctctacggcaaggtcagccctggccgatctcccttcacttggggccgcacccgcgagagctacggtactgaggttctttatgaggcgaacaacggccgtggcgctcctcaggatgacttctctgagggtgtcttcatcgactaccgtcacttcgaccgacgatctccaagcaccgatggaaagagctctcccaacaacaccgctgctcctctctacgagttcggtcacggtctatcttggtccacctttgagtactctgacctcaacatccagaagaacgtcgagaacccctactctcctcccgctggccagaccatccccgccccaacctttggcaacttcagcaagaacctcaacgactacgtgttccccaagggcgtccgatacatctacaagttcatctaccccttcctcaacacctcctcatccgccagcgaggcatccaacgatggtggccagtttggtaagactgccgaagagttcctccctcccaacgccctcaacggctcagcccagcctcgtcttcccgcctctggtgccccaggtggtaaccctcaattgtgggacatcttgtacaccgtcacagccacaatcaccaacacaggcaacgccacctccgacgagattccccagctgtatgtcagcctcggtggcgagaacgagcccatccgtgttctccgcggtttcgaccgtatcgagaacattgctcccggccagagcgccatcttcaacgctcaattgacccgtcgcgatctgagtaactgggatacaaatgcccagaactgggtcatcactgaccatcccaagactgtctgggttggaagcagctctcgcaagctgcctctcagcgccaagttggagtaagaaagccaaacaagggttgttttttggactgcaattttttgggaggacatagtagccgcgcgccagttacgtc

SEQ ID NO: 60 - protein, 899 aa, Fusarium verticillioides
MKLNWVAAALSIGAAGTDSAVALASAVPDTLAGVKKADAQKVVTRDTLAYSPPHYPSPWMDPNAVGWEEAYAKAKSFVSQLTLMEKVNLTTGVGWQGERCVGNVGSIPRLGMRGLCLQDGPLGIRLSDYNSAFPAGTTAGASWSKSLWYERGLLMGTEFKEKGIDIALGPATGPLGRTAAGGRNWEGFTVDPYMAGHAMAEAVKGIQDAGVIACAKHYIANEQEHFRQSGEVQSRKYNISESLSSNLDDKTMHELYAWPFADAVRAGVGSVMCSYNQINNSYGCQNSKLLNGILKDEMGFQGFVMSDWAAQHTGAASAVAGLDMSMPGDTAFDSGYSFWGGNLTLAVINGTVPAWRVDDMALRIMSAFFKVGKTIEDLPDINFSSWTRDTFGFVHTFAQENREQVNFGVNVQHDHKSHIREAAAKGSVVLKNTGSLPLKNPKFLAVIGEDAGPNPAGPNGCGDRGCDNGTLAMAWGSGTSQFPYLITPDQGLSNRATQDGTRYESILTNNEWASVQALVSQPNVTAIVFANADSGEGYIEVDGNFGDRKNLTLWQQGDELIKNVSSICPNTIVVLHTVGPVLLADYEKNPNITAIVWAGLPGQESGNAIADLLYGKVSPGRSPFTWGRTRESYGTEVLYEANNGRGAPQDDFSEGVFIDYRHFDRRSPSTDGKSSPNNTAAPLYEFGHGLSWSTFEYSDLNIQKNVENPYSPPAGQTIPAPTFGNFSKNLNDYVFPKGVRYIYKFIYPFLNTSSSASEASNDGGQFGKTAEEFLPPNALNGSAQPRLPASGAPGGNPQLWDILYTVTATITNTGNATSDEIPQLYVSLGGENEPIRVLRGFDRIENIAPGQSAIFNAQLTRRDLSNWDTNAQNWVITDHPKTVWVGSSSRKLPLSAKLE

SEQ ID NO: 61 - DNA, 2370 bp, Trichoderma reesei
atgcgttaccgaacagcagctgcgctggcacttgccactgggccctttgctagggcagacagtcagtatagctggtcccatactgggatgtgatatgtatcctggagacaccatgctgactcttgaatcaaggtagctcaacatcgggggcctcggctgaggcagttgtacctcctgcagggactccatggggaaccgcgtacgacaaggcgaaggccgcattggcaaagctcaatctccaagataaggtcggcatcgtgagcggtgtcggctggaacggcggtccttgcgttggaaacacatctccggcctccaagatcagctatccatcgctatgccttcaagacggacccctcggtgttcgatactcgacaggcagcacagcctttacgccgggcgttcaagcggcctcgacgtgggatgtcaatttgatccgcgaacgtggacagttcatcggtgaggaggtgaaggcctcggggattcatgtcatacttggtcctgtggctgggccgctgggaaagactccgcagggcggtcgcaactgggagggcttcggtgtcgatccatatctcacgggcattgccatgggtcaaaccatcaacggcatccagtcggtaggcgtgcaggcgacagcgaagcactatatcctcaacgagcaggagctcaatcgagaaaccatttcgagcaacccagatgaccgaactctccatgagctgtatacttggccatttgccgacgcggttcaggccaatgtcgcttctgtcatgtgctcgtacaacaaggtcaataccacctgggcctgcgaggatcagtacacgctgcagactgtgctgaaagaccagctggggttcccaggctatgtcatgacggactggaacgcacagcacacgactgtccaaagcgcgaattctgggcttgacatgtcaatgcctggcacagacttcaacggtaacaatcggctctggggtccagctctcaccaatgcggtaaatagcaatcaggtccccacgagcagagtcgacgatatggtgactcgtatcctcgccgcatggtacttgacaggccaggaccaggcaggctatccgtcgttcaacatcagcagaaatgttcaaggaaaccacaagaccaatgtcagggcaattgccagggacggcatcgttctgctcaagaatgacgccaacatcctgccgctcaagaagcccgctagcattgccgtcgttggatctgccgcaatcattggtaaccacgccagaaactcgccctcgtgcaacgacaaaggctgcgacgacggggccttgggcatgggttggggttccggcgccgtcaactatccgtacttcgtcgcgccctacgatgccatcaataccagagcgtcttcgcagggcacccaggttaccttgagcaacaccgacaacacgtcctcaggcgcatctgcagcaagaggaaaggacgtcgccatcgtcttcatcaccgccgactcgggtgaaggctacatcaccgtggagggcaacgcgggcgatcgcaacaacctggatccgtggcacaacggcaatgccctggtccaggcggtggccggtgccaacagcaacgtcattgttgttgtccactccgttggcgccatcattctggagcagattcttgctcttccgcaggtcaaggccgttgtctgggcgggtcttccttctcaggagagcggcaatgcgctcgtcgacgtgctgtggggagatgtcagcccttctggcaagctggtgtacaccattgcgaagagccccaatgactataacactcgcatcgtttccggcggcagtgacagcttcagcgagggactgttcatcgactataagcacttcgacgacgccaatatcacgccgcggtacgagttcggctatggactgtgtaagtttgctaacctgaacaatctattagacaggttgactgacggatgactgtggaatgatagcttacaccaagttcaactactcacgcctctccgtcttgtcgaccgccaagtctggtcctgcgactggggccgttgtgccgggaggcccgagtgatctgttccagaatgtcgcgacagtcaccgttgacatcgcaaactctggccaagtgactggtgccgaggtagcccagctgtacatcacctacccatcttcagcacccaggacccctccgaagcagctgcgaggctttgccaagctgaacctcacgcctggtcagagcggaacagcaacgttcaacatccgacgacgagatctcagctactgggacacggcttcgcagaaatgggtggtgccgtcggggtcgtttggcatcagcgtgggagcgagcagccgggatatcaggctgacgagcactctgtcggtagcgtag

SEQ ID NO: 62 - protein, 744 aa, Trichoderma reesei
MRYRTAAALALATGPFARADSHSTSGASAEAVVPPAGTPWGTAYDKAKAALAKLNLQDKVGIVSGVGWNGGPCVGNTSPASKISYPSLCLQDGPLGVRYSTGSTAFTPGVQAASTWDVNLIRERGQFIGEEVKASGIHVILGPVAGPLGKTPQGGRNWEGFGVDPYLTGIAMGQTINGIQSVGVQATAKHYILNEQELNRETISSNPDDRTLHELYTWPFADAVQANVASVMCSYNKVNTTWACEDQYTLQTVLKDQLGFPGYVMTDWNAQHTTVQSANSGLDMSMPGTDFNGNNRLWGPALTNAVNSNQVPTSRVDDMVTRILAAWYLTGQDQAGYPSFNISRNVQGNHKTNVRAIARDGIVLLKNDANILPLKKPASIAVVGSAAIIGNHARNSPSCNDKGCDDGALGMGWGSGAVNYPYFVAPYDAINTRASSQGTQVTLSNTDNTSSGASAARGKDVAIVFITADSGEGYITVEGNAGDRNNLDPWHNGNALVQAVAGANSNVIVVVHSVGAIILEQILALPQVKAVVWAGLPSQESGNALVDVLWGDVSPSGKLVYTIAKSPNDYNTRIVSGGSDSFSEGLFIDYKHFDDANITPRYEFGYGLSYTKFNYSRLSVLSTAKSGPATGAVVPGGPSDLFQNVATVTVDIANSGQVTGAEVAQLYITYPSSAPRTPPKQLRGFAKLNLTPGQSGTATFNIRRRDLSYWDTASQKWVVPSGSFGISVGASSRDIRLTSTLSVA

SEQ ID NO: 63 - DNA, 2625 bp, Trichoderma reesei
atgaagacgttgtcagtgtttgctgccgcccttttggcggccgtagctgaggccaatccctacccgcctcctcactccaaccaggcgtactcgcctcctttctacccttcgccatggatggaccccagtgctccaggctgggagcaagcctatgcccaagctaaggagttcgtctcgggcttgactctcttggagaaggtcaacctcaccaccggtgttggctggatgggtgagaagtgcgttggaaacgttggtaccgtgcctcgcttgggcatgcgaagtctttgcatgcaggacggccccctgggtctccgattcaacacgtacaacagcgctttcagcgttggcttgacggccgccgccagctggagccgacacctttgggttgaccgcggtaccgctctgggctccgaggcaaagggcaagggtgtcgatgttcttctcggacccgtggctggccctctcggtcgcaaccccaacggaggccgtaacgtcgagggtttcggctcggatccctatctggcgggtttggctctggccgataccgtgaccggaatccagaacgcgggcaccatcgcctgtgccaagcacttcctcctcaacgagcaggagcatttccgccaggtcggcgaagctaacggttacggataccccatcaccgaggctctgtcttccaacgttgatgacaagacgattcacgaggtgtacggctggcccttccaggatgctgtcaaggctggtgtcgggtccttcatgtgctcgtacaaccaggtcaacaactcgtacgcttgccaaaactccaagctcatcaacggcttgctcaaggaggagtacggtttccaaggctttgtcatgagcgactggcaggcccagcacacgggtgtcgcgtctgctgttgccggtctcgatatgaccatgcctggtgacaccgccttcaacaccggcgcatcctactttggaagcaacctgacgcttgctgttctcaacggcaccgtccccgagtggcgcattgacgacatggtgatgcgtatcatggctcccttcttcaaggtgggcaagacggttgacagcctcattgacaccaactttgattcttggaccaatggcgagtacggctacgttcaggccgccgtcaatgagaactgggagaaggtcaactacggcgtcgatgtccgcgccaaccatgcgaaccacatccgcgaggttggcgccaagggaactgtcatcttcaagaacaacggcatcctgccccttaagaagcccaagttcctgaccgtcattggtgaggatgctggcggcaaccctgccggccccaacggctgcggtgaccgcggctgtgacgacggcactcttgccatggagtggggatctggtactaccaacttcccctacctcgtcacccccgacgcggccctgcagagccaggctctccaggacggcacccgctacgagagcatcctgtccaactacgccatctcgcagacccaggcgctcgtcagccagcccgatgccattgccattgtctttgccaactcggatagcggcgagggctacatcaacgtcgatggcaacgagggcgaccgcaagaacctgacgctgtggaagaacggcgacgatctgatcaagactgttgctgctgtcaaccccaagacgattgtcgtcatccactcgaccggccccgtgattctcaaggactacgccaaccaccccaacatctctgccattctgtgggccggtgctcctggccaggagtctggcaactcgctggtcgacattctgtacggcaagcagagcccgggccgcactcccttcacctggggcccgtcgctggagagctacggagttagtgttatgaccacgcccaacaacggcaacggcgctccccaggataacttcaacgagggcgccttcatcgactaccgctactttgacaaggtggctcccggcaagcctcgcagctcggacaaggctcccacgtacgagtttggcttcggactgtcgtggtcgacgttcaagttctccaacctccacatccagaagaacaatgtcggccccatgagcccgcccaacggcaagacgattgcggctccctctctgggcagcttcagcaagaaccttaaggactatggcttccccaagaacgttcgccgcatcaaggagtttatctacccctacctgagcaccactacctctggcaaggaggcgtcgggtgacgctcactacggccagactgcgaaggagttcctccccgccggtgccctggacggcagccctcagcctcgctctgcggcctctggcgaacccggcggcaaccgccagctgtacgacattctctacaccgtgacggccaccattaccaacacgggctcggtcatggacgacgccgttccccagctgtacctgagccacggcggtcccaacgagccgcccaaggtgctgcgtggcttcgaccgcatcgagcgcattgctcccggccagagcgtcacgttcaaggcagacctgacgcgccgtgacctgtccaactgggacacgaagaagcagcagtgggtcattaccgactaccccaagactgtgtacgtgggcagctcctcgcgcgacctgccgctgagcgcccgcctgccatga

SEQ ID NO: 64 - protein, 874 aa, Trichoderma reesei
MKTLSVFAAALLAAVAEANPYPPPHSNQAYSPPFYPSPWMDPSAPGWEQAYAQAKEFVSGLTLLEKVNLTTGVGWMGEKCVGNVGTVPRLGMRSLCMQDGPLGLRFNTYNSAFSVGLTAAASWSRHLWVDRGTALGSEAKGKGVDVLLGPVAGPLGRNPNGGRNVEGFGSDPYLAGLALADTVTGIQNAGTIACAKHFLLNEQEHFRQVGEANGYGYPITEALSSNVDDKTIHEVYGWPFQDAVKAGVGSFMCSYNQVNNSYACQNSKLINGLLKEEYGFQGFVMSDWQAQHTGVASAVAGLDMTMPGDTAFNTGASYFGSNLTLAVLNGTVPEWRIDDMVMRIMAPFFKVGKTVDSLIDTNFDSWTNGEYGYVQAAVNENWEKVNYGVDVRANHANHIREVGAKGTVIFKNNGILPLKKPKFLTVIGEDAGGNPAGPNGCGDRGCDDGTLAMEWGSGTTNFPYLVTPDAALQSQALQDGTRYESILSNYAISQTQALVSQPDAIAIVFANSDSGEGYINVDGNEGDRKNLTLWKNGDDLIKTVAAVNPKTIVVIHSTGPVILKDYANHPNISAILWAGAPGQESGNSLVDILYGKQSPGRTPFTWGPSLESYGVSVMTTPNNGNGAPQDNFNEGAFIDYRYFDKVAPGKPRSSDKAPTYEFGFGLSWSTFKFSNLHIQKNNVGPMSPPNGKTIAAPSLGSFSKNLKDYGFPKNVRRIKEFIYPYLSTTTSGKEASGDAHYGQTAKEFLPAGALDGSPQPRSAASGEPGGNRQLYDILYTVTATITNTGSVMDDAVPQLYLSHGGPNEPPKVLRGFDRIERIAPGQSVTFKADLTRRDLSNWDTKKQQWVITDYPKTVYVGSSSRDLPLSARLP

SEQ ID NO: 65 - DNA, 2577 bp, Artificial Sequence
  synthetic codon optimized sequence from Talaromyces emersonii
atgcgcaacggcctcctcaaggtcgccgccttagccgctgccagcgccgtcaacggcgagaacctcgcctacagcccccccttctaccccagcccctgggccaacggccagggcgactgggccgaggcctaccagaaggccgtccagttcgtcagccagctcaccctcgccgagaaggtcaacctcaccaccggcaccggctgggagcaggaccgctgcgtcggccaggtcggcagcatcccccgcttaggcttccccggcctctgcatgcaggacagccccctcggcgtccgcgacaccgactacaacagcgccttccctgccggcgttaacgtcgccgccacctgggaccgcaacttagcctaccgcagaggcgtcgccatgggcgaggaacaccgcggcaagggcgtcgacgtccagttaggccccgtcgccggccccttaggccgctctcctgatgccggccgcaactgggagggcttcgcccccgaccccgtcctcaccggcaacatgatggccagcaccatccagggcatccaggatgctggcgtcattgcctgcgccaagcacttcatcctctacgagcaggaacacttccgccagggcgcccaggacggctacgacatcagcgacagcatcagcgccaacgccgacgacaagaccatgcacgagttatacctctggcccttcgccgatgccgtccgcgccggtgtcggcagcgtcatgtgcagctacaaccaggtcaacaacagctacgcctgcagcaacagctacaccatgaacaagctcctcaagagcgagttaggcttccagggcttcgtcatgaccgactggggcggccaccacagcggcgtcggctctgccctcgccggcctcgacatgagcatgcccggcgacattgccttcgacagcggcacgtctttctggggcaccaacctcaccgttgccgtcctcaacggctccatccccgagtggcgcgtcgacgacatggccgtccgcatcatgagcgcctactacaaggtcggccgcgaccgctacagcgtccccatcaacttcgacagctggaccctcgacacctacggccccgagcactacgccgtcggccagggccagaccaagatcaacgagcacgtcgacgtccgcggcaaccacgccgagatcatccacgagatcggcgccgcctccgccgtcctcctcaagaacaagggcggcctccccctcactggcaccgagcgcttcgtcggtgtctttggcaaggatgctggcagcaacccctggggcgtcaacggctgcagcgaccgcggctgcgacaacggcaccctcgccatgggctggggcagcggcaccgccaactttccctacctcgtcacccccgagcaggccatccagcgcgaggtcctcagccgcaacggcaccttcaccggcatcaccgacaacggcgccttagccgagatggccgctgccgcctctcaggccgacacctgcctcgtctttgccaacgccgactccggcgagggctacatcaccgtcgatggcaacgagggcgaccgcaagaacctcaccctctggcagggcgccgaccaggtcatccacaacgtcagcgccaactgcaacaacaccgtcgtcgtcttacacaccgtcggccccgtcctcatcgacgactggtacgaccaccccaacgtcaccgccatcctctgggccggtttacccggtcaggaaagcggcaacagcctcgtcgacgtcctctacggccgcgtcaaccccggcaagacccccttcacctggggcagagcccgcgacgactatggcgcccctctcatcgtcaagcctaacaacggcaagggcgccccccagcaggacttcaccgagggcatcttcatcgactaccgccgcttcgacaagtacaacatcacccccatctacgagttcggcttcggcctcagctacaccaccttcgagttcagccagttaaacgtccagcccatcaacgcccctccctacacccccgccagcggctttacgaaggccgcccagagcttcggccagccctccaatgccagcgacaacctctaccctagcgacatcgagcgcgtccccctctacatctacccctggctcaacagcaccgacctcaaggccagcgccaacgaccccgactacggcctccccaccgagaagtacgtcccccccaacgccaccaacggcgacccccagcccattgaccctgccggcggtgcccctggcggcaaccccagcctctacgagcccgtcgcccgcgtcaccaccatcatcaccaacaccggcaaggtcaccggcgacgaggtcccccagctctatgtcagcttaggcggccctgacgacgcccccaaggtcctccgcggcttcgaccgcatcaccctcgcccctggccagcagtacctctggaccaccaccctcactcgccgcgacatcagcaactgggaccccgtcacccagaactgggtcgtcaccaactacaccaagaccatctacgtcggcaacagcagccgcaacctccccctccaggcccccctcaagccctaccccggcatctgatga

SEQ ID NO: 66 - protein, 857 aa, Talaromyces emersonii
MRNGLLKVAALAAASAVNGENLAYSPPFYPSPWANGQGDWAEAYQKAVQFVSQLTLAEKVNLTTGTGWEQDRCVGQVGSIPRLGFPGLCMQDSPLGVRDTDYNSAFPAGVNVAATWDRNLAYRRGVAMGEEHRGKGVDVQLGPVAGPLGRSPDAGRNWEGFAPDPVLTGNMMASTIQGIQDAGVIACAKHFILYEQEHFRQGAQDGYDISDSISANADDKTMHELYLWPFADAVRAGVGSVMCSYNQVNNSYACSNSYTMNKLLKSELGFQGFVMTDWGGHHSGVGSALAGLDMSMPGDIAFDSGTSFWGTNLTVAVLNGSIPEWRVDDMAVRIMSAYYKVGRDRYSVPINFDSWTLDTYGPEHYAVGQGQTKINEHVDVRGNHAEIIHEIGAASAVLLKNKGGLPLTGTERFVGVFGKDAGSNPWGVNGCSDRGCDNGTLAMGWGSGTANFPYLVTPEQAIQREVLSRNGTFTGITDNGALAEMAAAASQADTCLVFANADSGEGYITVDGNEGDRKNLTLWQGADQVIHNVSANCNNTVVVLHTVGPVLIDDWYDHPNVTAILWAGLPGQESGNSLVDVLYGRVNPGKTPFTWGRARDDYGAPLIVKPNNGKGAPQQDFTEGIFIDYRRFDKYNITPIYEFGFGLSYTTFEFSQLNVQPINAPPYTPASGFTKAAQSFGQPSNASDNLYPSDIERVPLYIYPWLNSTDLKASANDPDYGLPTEKYVPPNATNGDPQPIDPAGGAPGGNPSLYEPVARVTTIITNTGKVTGDEVPQLYVSLGGPDDAPKVLRGFDRITLAPGQQYLWTTTLTRRDISNWDPVTQNWVVTNYTKTIYVGNSSRNLPLQAPLKPYPGI

SEQ ID NO: 67 - DNA, 2586 bp, Aspergillus niger
atgcgcttcaccagcatcgaggccgtcgccctcaccgccgtcagcctcgccagcgccgacgagttagcctacagccccccctactaccccagcccctgggccaacggccagggcgactgggccgaggcctaccagcgcgccgtcgacatcgtcagccagatgaccctcgccgagaaggtcaacctcaccaccggcaccggctgggagttagagttatgcgtcggccagactggtggcgtcccccgcctcggcatccccggcatgtgcgcccaggacagccccctcggcgtccgcgacagcgactacaacagcgccttccctgccggcgtcaacgtcgccgccacctgggacaagaacctcgcctacctccgcggccaggccatgggccaggaattcagcgacaagggcgccgacatccagttaggccccgctgccggccctttaggccgctctcccgacggcggcagaaactgggagggcttcagccccgaccccgctctcagcggcgtcctcttcgccgagactatcaagggcatccaggatgctggcgtcgtcgccaccgccaagcactacattgcctacgagcaggaacacttccgccaggcccccgaggcccagggctacggcttcaacatcaccgagagcggcagcgccaacctcgacgacaagaccatgcacgagttatacctctggcccttcgccgacgccattagagctggcgctggtgctgtcatgtgcagctacaaccagatcaacaacagctacggctgccagaacagctacaccctcaacaagctcctcaaggccgagttaggcttccagggcttcgtcatgtccgactgggccgcccaccacgccggcgtcagcggcgccttagccggcctcgacatgagcatgcccggcgacgtcgactacgacagcggcaccagctactggggcaccaacctcaccatcagcgtcctcaacggcaccgtcccccagtggcgcgtcgacgacatggccgtccgcatcatggccgcctactacaaggtcggccgcgaccgcctctggaccccccccaacttcagcagctggacccgcgacgagtacggcttcaagtactactacgtcagcgagggcccctatgagaaggtcaaccagttcgtcaacgtccagcgcaaccacagcgagttaatccgccgcatcggcgccgacagcaccgtcctcctcaagaacgacggcgccctccccctcaccggcaaggaacgcctcgtcgccctcatcggcgaggacgccggcagcaacccctacggcgccaacggctgcagcgaccgcggctgcgacaacggcaccctcgccatgggctggggcagcggcaccgccaacttcccttacctcgtcacccccgagcaggccatcagcaacgaggtcctcaagaacaagaacggcgtctttaccgccaccgacaactgggccatcgaccagatcgaggccttagccaagaccgcctctgtcagcctcgtctttgtcaacgccgacagcggcgagggctacatcaacgtcgacggcaacctcggcgaccgccgcaacctcaccctctggcgcaacggcgacaacgtcatcaaggccgccgccagcaactgcaacaacaccatcgtcatcatccacagcgtcggccccgtcctcgtcaacgagtggtacgacaaccccaacgtcaccgccatcctctggggcggcttacccggccaggaaagcggcaacagcctcgccgacgtcctctacggccgcgtcaaccctggcgccaagagccccttcacctggggcaagacccgcgaggcctatcaggactacctctacaccgagcccaacaacggcaacggcgccccccaggaagatttcgtcgagggcgtctttatcgactaccgcggctttgacaagcgcaacgagactcccatctacgagttcggctacggcctcagctacaccaccttcaactacagcaacctccaggtcgaggtcctcagcgcccctgcctacgagcccgccagcggcgagactgaggccgcccccaccttcggcgaggtcggcaacgccagcgactacttataccccgacggcctccagcgcatcaccaagttcatctacccctggctcaacagcaccgacctcgaggccagcagcggcgacgcctcttacggccaggacgcctccgactacctccccgagggtgccaccgacggcagcgctcagcccatcttacctgccggtggcggtgctggcggcaaccccagactctacgacgagctgatccgcgtcagcgtcaccatcaagaacaccggcaaggtcgctggtgacgaggtcccccagctctacgtcagcttaggcggccctaacgagcccaagatcgtcctccgccagttcgagcgcatcaccctccagcccagcaaggaaactcagtggagcaccaccctcactcgccgcgacctcgccaactggaacgtcgagactcaggactgggagatcaccagctaccccaagatggtctttgccggcagcagcagccgcaagctccccctccgcgccagcctccccaccgtccactgatga

SEQ ID NO: 68 - protein, 860 aa, Aspergillus niger
MRFTSIEAVALTAVSLASADELAYSPPYYPSPWANGQGDWAEAYQRAVDIVSQMTLAEKVNLTTGTGWELELCVGQTGGVPRLGIPGMCAQDSPLGVRDSDYNSAFPAGVNVAATWDKNLAYLRGQAMGQEFSDKGADIQLGPAAGPLGRSPDGGRNWEGFSPDPALSGVLFAETIKGIQDAGVVATAKHYIAYEQEHFRQAPEAQGYGFNITESGSANLDDKTMHELYLWPFADAIRAGAGAVMCSYNQINNSYGCQNSYTLNKLLKAELGFQGFVMSDWAAHHAGVSGALAGLDMSMPGDVDYDSGTSYWGTNLTISVLNGTVPQWRVDDMAVRIMAAYYKVGRDRLWTPPNFSSWTRDEYGFKYYYVSEGPYEKVNQFVNVQRNHSELIRRIGADSTVLLKNDGALPLTGKERLVALIGEDAGSNPYGANGCSDRGCDNGTLAMGWGSGTANFPYLVTPEQAISNEVLKNKNGVFTATDNWAIDQIEALAKTASVSLVFVNADSGEGYINVDGNLGDRRNLTLWRNGDNVIKAAASNCNNTIVIIHSVGPVLVNEWYDNPNVTAILWGGLPGQESGNSLADVLYGRVNPGAKSPFTWGKTREAYQDYLYTEPNNGNGAPQEDFVEGVFIDYRGFDKRNETPIYEFGYGLSYTTFNYSNLQVEVLSAPAYEPASGETEAAPTFGEVGNASDYLYPDGLQRITKFIYPWLNSTDLEASSGDASYGQDASDYLPEGATDGSAQPILPAGGGAGGNPRLYDELIRVSVTIKNTGKVAGDEVPQLYVSLGGPNEPKIVLRQFERITLQPSKETQWSTTLTRRDLANWNVETQDWEITSYPKMVFAGSSSRKLPLRASLPTVH

SEQ ID NO: 69 - DNA, 3203 bp, Fusarium oxysporum
atgaagctgaactgggtcgccgcagccctctctataggtgctgctggcactgatggtgcagttgctcttgcttctgaagttccaggcactttggctggtgtaaaggtcggtttttttaccatttcctcacctaatctcagccttgttgccatatcgcccttattcgctcggacgctacgcaccaaatcgcgatcatttcctcccttgcagccttgttttcttttttcgatcttccctccgcaatcgccagcacccttagcctacacaaaaacccccgagacagtctcattgagtttgtcgacatcaagttgcttctcaagtgtgcatttgcgtggctgtctacttctgcctctagaccaccaaatctgggcgcaattgatcgctcaaaccttgttcgaataagccttttattcgagacgtccaatttttacagagaatgtacctttcaataataccgacgttatgcgcggcggtggctgctgtgatggttgttgatcagaatactgacgctcaaaaggttgtcacgagagatacactcgcacactcacctcctcactatccttcaccatggatggatcctaatgccattggctgggaggaagcttacgccaaagcaaagaactttgtgtcccagctcactctcctcgaaaaggtcaacttgaccactggtgttgggtaagtagctccttgcgaacagtgcatctcggtctccttgactaacgactctctcaggtggcaaggcgaacgctgtgtaggaaacgtgggatcaattcctcgtcttggtatgcgaggtctttgtcttcaggatggtcctcttggaattcgtctgtccgattacaacagtgcttttcccgctggcaccacagctggtgcttcttggagcaagtctctctggtatgagaggggtcttctgatgggaactgagttcaaggggaagggtatcgatatcgctcttggccctgctactggtcctcttggccgcactgctgctggtggacgaaactgggagggctttaccgttgatccttatatggctggccatgccatggccgaggccgtcaagggcatccaagacgcaggtgtcattgcttgtgctaagcattacatcgcaaacgagcaaggtaagccaattggacggtttgggaaatcgacagagaactgacccccttgtagagcacttccgacagagtggcgaggtccagtcccgcaagtacaacatctccgagtctctctcctccaacctggacgacaagactttgcacgagctctacgcctggccctttgctgatgccgtccgcgctggcgtcggttcagtcatgtgctcttacaatcagatcaacaactcgtacggttgccagaactccaagctcctcaacggtatcctcaaggacgagatgggtttccagggcttcgtcatgagcgattgggcggcccagcacaccggtgctgcttctgccgtcgctggtcttgatatgagcatgcctggtgacaccgcgttcgacagtggatatagcttctggggtggaaacctgactcttgctgtcatcaacggaactgttcccgcctggcgagttgatgacatggctctgcgaatcatgtcggccttcttcaaggttggaaagacggtagaggacctccccgacatcaacttctcctcctggacccgcgacaccttcggcttcgtccaaacatttgctcaagagaaccgcgaacaagtcaactttggagttaacgtccagcacgaccacaagaaccacatccgtgagtctgccgccaagggaagcgtcatcctcaagaacaccggctcccttcccctcaacaatcccaagttcctcgctgtcattggtgaggacgccggtcccaaccctgctggacccaatggttgcggcgaccgtggttgcgacaatggtaccctggctatggcttggggctcgggaacttctcaattcccttacttgatcacacccgaccaaggtctccagaaccgagctgcccaagacggaactcgatatgagagcatcttgaccaacaacgaatgggcccagacacaggctcttgtcagccaacccaacgtgaccgctatcgtttttgccaacgccgactctggtgagggttacattgaagtcgacggaaacttcggtgatcgcaagaacctcaccctctggcaacagggagacgagctcatcaagaacgtctcgtccatctgccccaacaccattgtcgttctgcataccgtcggccctgtcctgctcgccgactacgagaagaaccccaacatcaccgccatcgtctgggctggtcttcccggccaagagtctggcaatgccatcgctgatctcctctacggcaaggtaagccctggccgatctcccttcacttggggccgcacccgtgagagctacggtaccgaggttctttatgaggcgaacaacggccgtggcgctcctcaggatgacttctcggagggtgtcttcattgactaccgtcactttgatcgacgatctcccagcaccgatggcaagagcgctcccaacaacaccgctgctcctctctacgagttcggtcatggtctgtcttggactacctttgagtattcagacctcaacatccagaagaacgttaactccacctactctcctcctgctggtcagaccattcctgccccaacctttggcaacttcagcaagaacctcaacgactacgtgttccctaagggtgtccgatacatctacaagttcatctaccccttcctgaacacttcctcatccgccagcgaggcatctaacgacggcggccagtttggtaagactgccgaagagttcctacctccaaacgccctcaacggctcagcccagcctcgtcttccctcttctggtgccccaggcggtaaccctcaattgtgggatatcctgtacaccgtcacagccacaatcaccaacacaggcaacgccacctccgacgagattccccagctgtatgtcagcctcggtggcgagaacgaacccgttcgtgtcctccgcggtttcgaccgtatcgagaacattgctcccggccagagcgccatcttcaacgctcaattgacccgtcgcgatctgagcaactgggatgtggatgcccagaactgggttatcaccgaccatccaaagacggtgtgggttggaagtagttctcgcaagctgcctctcagcgccaagttggaataa

SEQ ID NO: 70 - protein, 899 aa, Fusarium oxysporum
MKLNWVAAALSIGAAGTDGAVALASEVPGTLAGVKNTDAQKVVTRDTLAHSPPHYPSPWMDPNAIGWEEAYAKAKNFVSQLTLLEKVNLTTGVGWQGERCVGNVGSIPRLGMRGLCLQDGPLGIRLSDYNSAFPAGTTAGASWSKSLWYERGLLMGTEFKGKGIDIALGPATGPLGRTAAGGRNWEGFTVDPYMAGHAMAEAVKGIQDAGVIACAKHYIANEQEHFRQSGEVQSRKYNISESLSSNLDDKTLHELYAWPFADAVRAGVGSVMCSYNQINNSYGCQNSKLLNGILKDEMGFQGFVMSDWAAQHTGAASAVAGLDMSMPGDTAFDSGYSFWGGNLTLAVINGTVPAWRVDDMALRIMSAFFKVGKTVEDLPDINFSSWTRDTFGFVQTFAQENREQVNFGVNVQHDHKNHIRESAAKGSVILKNTGSLPLNNPKFLAVIGEDAGPNPAGPNGCGDRGCDNGTLAMAWGSGTSQFPYLITPDQGLQNRAAQDGTRYESILTNNEWAQTQALVSQPNVTAIVFANADSGEGYIEVDGNFGDRKNLTLWQQGDELIKNVSSICPNTIVVLHTVGPVLLADYEKNPNITAIVWAGLPGQESGNAIADLLYGKVSPGRSPFTWGRTRESYGTEVLYEANNGRGAPQDDFSEGVFIDYRHFDRRSPSTDGKSAPNNTAAPLYEFGHGLSWTTFEYSDLNIQKNVNSTYSPPAGQTIPAPTFGNFSKNLNDYVFPKGVRYIYKFIYPFLNTSSSASEASNDGGQFGKTAEEFLPPNALNGSAQPRLPSSGAPGGNPQLWDILYTVTATITNTGNATSDEIPQLYVSLGGENEPVRVLRGFDRIENIAPGQSAIFNAQLTRRDLSNWDVDAQNWVITDHPKTVWVGSSSRKLPLSAKLE

SEQ ID NO: 71 - DNA, 3134 bp, Gibberella zeae
atgaaggccaattggcttgccgcggccgtttatttggctgctggcaccgatgctgcagtccctgacactttggcaggagtcaatgtaagctactcttcaatttcatctcatctcaactttgccaggccacaacaacttttcttcactcacgatcttttcaccataaacgcaacagtttcacaaaaaataaagcccaaatcatgtctctgatcgttgaactcgccatcttcgtttacatcgcggttgtctttttcttcttgtacttctcattcgttgttgttctctacattttcgactggctgtttagccttgagattcttctcactccccgtgatgcctagatcactctctgaggcgtttaatctacttgtagagatgcgcctctcatttgttgtgtcgctagtcgcgatagttgctggaattgcagtccttgatcttcctactgacactcaaaagctcgttgcgcgggacacactcgctcactctcctcctcactatccctcgccatggatggaccctaacgctgtcggctgggaggacgcctacgccaaggccaaggactttgtctcccagatgactctcctagaaaaggtcaacttgaccactggtgttgggtaagtaacgagcgacaagacgtctacaatccactaacacgatctctagatggcagggcgaacgttgtgttggaaacgtgggatctatccctcgtctcggtatgcgaggcctctgtctccaggatggtcctctcggaattcgcttctccgactacaacagcgctttccctactggtgtcaccgctggtgcttcttggagtaaggccctttggtacgagcgaggacgattgatgggtaccgagtttaaggagaagggtatcgatattgctctcggccctgcaactggtcctctcggtcgccacgctgctggtggacgaaactgggaaggcttcactgtcgacccctacgccgctggccatgctatggctgagactgtcaagggtatccaagattctggagtcattgcttgtgctaagcattacatcgcaaacgagcaaggtatgtacaggcccattcaatggcttcaggaacgaaaactaactcttaatagaacacttccgtcaacgaggcgatgtcatgtctcaaaagttcaacatttccgagtctctgtcttccaaccttgacgataagactatgcacgagctctacaactggcctttcgccgacgccgtccgcgccggtgttggctccattatgtgctcttacaaccaggtcaacaactcatatgcttgccagaactccaagctcctcaacggcatcctcaaggacgagatgggtttccagggtttcgtcatgagcgattggcaggctcagcacaccggtgccgcctccgctgttgccggtcttgacatgaccatgcctggtgacaccgagttcaacactggcttcagcttctggggtggaaacctgaccctcgctgttatcaacggtactgttcccgcctggagaatcgacgacatggctacccgaattatggctgctttcttcaaggttggccgatctgttgaggaggaacccgacatcaacttctcagcttggactcgtgatgagtatggcttcgtccagacctacgcccaagagaaccgagaaaaggtcaactttgctgttaatgtccagcacgaccacaagcgccacattcgcgaggctggcgcaaagggatccgtcgtcctcaagaacactggctcacttcctcttaagaagccccagttcctcgctgtcattggagaggacgctggttccaaccctgccggacccaacggttgcgctgaccgtggatgcgacaacggtactcttgccatggcatggggttccggaacctctcaattcccctaccttgtcacccccgaccaaggcatctcgctccaggctattcaggacggtactcgttatgagagcatcctcaacaacaaccagtggccccagacacaagctcttgtcagccagcccaacgtcaccgccattgtctttgccaatgccgattctggtgagggctacatcgaggttgacggcaactacggcgaccgcaagaacctcactctgtggaagcaaggcgatgagctcatcaagaacgtctctgctatctgccccaacaccattgtggtccttcacaccgttggccccgtccttctaaccgagtggcacaacaaccccaacatcaccgccattgtttgggctggtgtgcctggacaggagtccggtaacgccatcgccgacatcctctacggcaagaccagccctggacgttctcccttcacctggggtcgcacttatgacagctatggcaccaaggttctctacaaggccaacaatggagagggtgcccctcaagaggactttgtcgagggcaacttcatcgactaccgccactttgaccgacaatcccccagcaccaacggaaagagtgccaccaacgactcttctgctcctctctacgagttcggtttcggtctgtcctggactacctttgagtactctgatctcaaagtcgagtctgtcagcaacgcctcttacagcccctctgtcggaaacaccattcctgcccctacctacggcaacttcagcaagaacctggacgattacacattcccctcaggtgtccgatacctctacaagttcatctacccctacctcaacacctcttcctccgctgagaaggcttccggcgatgtcaagggcagatttggtgagaccggcgacgagttcctccctcccaacgctctcaacggttcatcgcagcctcgtcttccttccagtggtgctcccggcggtaaccctcagctctgggacattatgtacaccgtcactgccaccatcaccaacactggtgacgctacctcggatgaggttccccagctgtacgtcagcctcggtggtgagggcgagcctgtccgtgtcctccgtggcttcgagcgtcttgaaaacattgctcctggtgagagtgccacattcaccgctcagcttactcgccgtgacctgagcaactgggacgtcaacgtccagaactgggtcatcaccgatcacgccaagaagatctgggtcggcagcagctctcgcaatctgcccctcagcgccgacctgtag

SEQ ID NO: 72 - protein, 886 aa, Gibberella zeae
MKANWLAAAVYLAAGTDAAVPDTLAGVNLVARDTLAHSPPHYPSPWMDPNAVGWEDAYAKAKDFVSQMTLLEKVNLTTGVGWQGERCVGNVGSIPRLGMRGLCLQDGPLGIRFSDYNSAFPTGVTAGASWSKALWYERGRLMGTEFKEKGIDIALGPATGPLGRHAAGGRNWEGFTVDPYAAGHAMAETVKGIQDSGVIACAKHYIANEQEHFRQRGDVMSQKFNISESLSSNLDDKTMHELYNWPFADAVRAGVGSIMCSYNQVNNSYACQNSKLLNGILKDEMGFQGFVMSDWQAQHTGAASAVAGLDMTMPGDTEFNTGFSFWGGNLTLAVINGTVPAWRIDDMATRIMAAFFKVGRSVEEEPDINFSAWTRDEYGFVQTYAQENREKVNFAVNVQHDHKRHIREAGAKGSVVLKNTGSLPLKKPQFLAVIGEDAGSNPAGPNGCADRGCDNGTLAMAWGSGTSQFPYLVTPDQGISLQAIQDGTRYESILNNNQWPQTQALVSQPNVTAIVFANADSGEGYIEVDGNYGDRKNLTLWKQGDELIKNVSAICPNTIVVLHTVGPVLLTEWHNNPNITAIVWAGVPGQESGNAIADILYGKTSPGRSPFTWGRTYDSYGTKVLYKANNGEGAPQEDFVEGNFIDYRHFDRQSPSTNGKSATNDSSAPLYEFGFGLSWTTFEYSDLKVESVSNASYSPSVGNTIPAPTYGNFSKNLDDYTFPSGVRYLYKFIYPYLNTSSSAEKASGDVKGRFGETGDEFLPPNALNGSSQPRLPSSGAPGGNPQLWDIMYTVTATITNTGDATSDEVPQLYVSLGGEGEPVRVLRGFERLENIAPGESATFTAQLTRRDLSNWDVNVQNWVITDHAKKIWVGSSSRNLPLSADL

SEQ ID NO: 73 - DNA, 2796 bp, Nectria haematococca
atgcggttcaccgtccttctcgcggcattttcggggcttgtccccatggttggttcgcaagctgaccagaaaccactacagctcggtgtgaacaataacactctggcgcattcacctcctcactatccttcgccatggatggatcctgctgctcctggctgggaggaagcctatctcaaggcgaaagattttgtttcacagcttacccttcttgaaaaggtcaacttgaccactggtgttgggtgagtcacttgttttcctctctcctgacgtgacactttgctttggcctgcttcctatatcgtctactagcattgctaacactcgaggcagatggatgggcgaacgttgcgtcggcaacgtgggttcactccctcgttttggaatgcgtggtctctgcatgcaggatggccccctcggcatccgcttgtctgactataactctgcctttcctactggtattacagctggtgcctcttggagccgtgccctttggtaccaacgtggcctcctgatgggcaccgagcatcgtgaaaaaggcatcgacgttgcacttgggcctgctactggtcctcttggtcgtactcctactggcggccgcaactgggagggtttctcggttgatccctacgttgctggcgttgccatggccgagactgttagcggcattcaagatggtggtactatcgcctgtgctaagcactacatcggcaacgaacaaggtatgcctcttcacttctcctcgctgataaatctgctcacaacaacctagagcaccatcgccaagcccccgaatccattggccgcggctacaacatcaccgagtccctgtcgtcgaacgttgatgacaagaccctccacgagctctatctctggccgttcgcagatgccgtcaaggctggtgttggtgctatcatgtgttcctaccagcagctgaacaactcttacggttgccaaaactctaagcttctcaacggaattctcaaggacgagctaggattccagggcttcgtcatgagtgactggcaagcccaacatgctggagctgctaccgctgttgcaggccttgacatgaccatgcccggtgacactttgttcaacaccggatacagcttctggggtggtaacctgaccctcgctgtagtcaatggcactgttcccgactggcgtattgacgacatggctatgagaatcatggcagctttcttcaaggttggcaagactgttgaggaccttcctgacatcaacttttcttcttggtctcgagacacttttggctacgttcaagccgctgcccaagagaactgggaacagatcaacttcggagttgatgttcgtcacgaccacagcgaacacattcgactctcggccgccaagggcaccgtcctccttaagaactctggctcattgcctctgaagaagcccaagttccttgccgtcgttggcgaggacgccggcccgaaccctgctggccccaacggctgtaacgaccgcggatgtaacaacggcactctggccatgtcctggggctcaggaacagcccagttcccttacctcgttactcccgactcagcgctacagaaccaggctgtcctcgacggcactcgctacgagagtgtcttgcggaacaaccagtgggaacagacacgcagtctcattagccaacctaacgtgacggctattgtgtttgccaatgccaattccggagagggatatatcgatgttgacggcaacgaaggcgatcggaagaatttgaccttgtggaacgagggtgatgacctaattaagaacgtctcctcaatctgccccaacaccattgttgttctgcacactgttggccctgtcatcctgacggaatggtatgacaacccgaacattaccgccatagtgtgggctggtgtacctggacaggagtccggcaatgctcttgtggacatcctttatggcaaaacaagccctggtcgctctcccttcacatggggtcgcacccgaaagagttacggcactgatgtcctatacgagcccaacaatggtcagggtgctcctcaagatgatttcacggagggagtctttatcgactatcgtcattttgaccaggtttctcctagcaccgacggcagcaagtctaatgatgagtccagtcccatctacgagtttggccatggtctgtcctggaccacgtttgagtactctgaactcaacattcaagctcacaacaagattcccttcgatcctcctattggcgagacgattgccgctccggtccttggcaactacagtaccgaccttgccgattacacgttccccgatggaattcgctacatctaccagttcatctatccctggttgaatacttcttcttccggaagagaggcttctggcgatcccgactacggaaagacggccgaagagttcctgccccccggagctctcgacgggtcagctcagccgcgacctccatcctctggtgctccaggtggaaaccctcatctttgggatgtgttgtacactgttagtgctatcatcaccaacactggcaacgccacctcggacgagatcccgcagctctacgttagtctcggtggcgagaacgagcccgtccgcgtccttcgcgggttcgaccgaattgagaacattgcgcctggccagagtgtcagattcacaactgacatcactcgccgcgacctgagcaactgggacgtcgtctctcagaactgggtcattacagactacgagaagaccgtatatgtcgggagcagctcccgcaacctgcctctcaaggcaaccctgaagtaa

SEQ ID NO: 74 - protein, 880 aa, Nectria haematococca
MRFTVLLAAFSGLVPMVGSQADQKPLQLGVNNNTLAHSPPHYPSPWMDPAAPGWEEAYLKAKDFVSQLTLLEKVNLTTGVGWMGERCVGNVGSLPRFGMRGLCMQDGPLGIRLSDYNSAFPTGITAGASWSRALWYQRGLLMGTEHREKGIDVALGPATGPLGRTPTGGRNWEGFSVDPYVAGVAMAETVSGIQDGGTIACAKHYIGNEQEHHRQAPESIGRGYNITESLSSNVDDKTLHELYLWPFADAVKAGVGAIMCSYQQLNNSYGCQNSKLLNGILKDELGFQGFVMSDWQAQHAGAATAVAGLDMTMPGDTLFNTGYSFWGGNLTLAVVNGTVPDWRIDDMAMRIMAAFFKVGKTVEDLPDINFSSWSRDTFGYVQAAAQENWEQINFGVDVRHDHSEHIRLSAAKGTVLLKNSGSLPLKKPKFLAVVGEDAGPNPAGPNGCNDRGCNNGTLAMSWGSGTAQFPYLVTPDSALQNQAVLDGTRYESVLRNNQWEQTRSLISQPNVTAIVFANANSGEGYIDVDGNEGDRKNLTLWNEGDDLIKNVSSICPNTIVVLHTVGPVILTEWYDNPNITAIVWAGVPGQESGNALVDILYGKTSPGRSPFTWGRTRKSYGTDVLYEPNNGQGAPQDDFTEGVFIDYRHFDQVSPSTDGSKSNDESSPIYEFGHGLSWTTFEYSELNIQAHNKIPFDPPIGETIAAPVLGNYSTDLADYTFPDGIRYIYQFIYPWLNTSSSGREASGDPDYGKTAEEFLPPGALDGSAQPRPPSSGAPGGNPHLWDVLYTVSAIITNTGNATSDEIPQLYVSLGGENEPVRVLRGFDRIENIAPGQSVRFTTDITRRDLSNWDVVSQNWVITDYEKTVYVGSSSRNLPLK

SEQ ID NO: 75 - DNA, 3169 bp, Verticillium dahliae
atgaagctgaccctcgctactgccttactggcagccagcgggtgtgtctctgcgggacaacccaagctcaaggtacgtacttgcctctttttcacaaggaaaccaaacccgcaccataatggtgattgagcagtcgtgctttcctcaacccgaatcaaacccatgccgtgttcgcgcatgccctttcgatcgtctgttgtgtgtgaacccacgctcttcaagcatcgcacatagcaccactccatcttcattttcgagcaatttcgggccgcagagagcggtctttcacttcaccacaatcgttcatgcctcgtgccccactgccatgtttcttcccagtattctacttctgagagccttgaccaccgttgtcgacatctcgtcgccaaggctcgttgacacggactctgtttcccttggaattaatattcgaaacaatgctgaccagcatcctcagcgccagactaacagctctagcgagctcgccttttcccctccgcactacccttctccatggatgaacccccaagcgactgggtgggaggacgcctacgcccgtgccagagaggtggtagagcagatgactctgctcgaaaaggtcaacctgacgacaggtgtcgggtaagcttcacagaccccgtcttgccatccaaagtcatctgacagaatcctagctggagcggtgatctctgcgtcggaaacgtcggctcgatcccccgaatcggctggagggggctttgtttgcaggatggcccacagggtatccgtttcgcggactacgtctcgtacttcacttcgagccagacagccggcgctacctgggaccgagggcttctgtaccagcgcgctcacgccattggcgccgaaggagtagccaagggcgtcgacgtcgtcctcgggcccgccattggccctctaggtcgccttcccgccggaggtcgtaactgggagggtttcgccgtggacccttacctcagtggcgttgctgtcgccgaatccgtcaggggcatccaggatgctggtgctattgccaacgtcaagcactacatcgtcaatgagcaggaacatttccgccaggctggcgaggctcaaggttacggctacgatgtcgacgaggcattatcgtcgaacgttgacgacaagaccatgcatgagctttacctttggccatttgcagacgctgtccgtgctggagccggcagtgtcatgtgttcttatcaacaggtgggggcaataccattctctcctctttccttgcagacagtgcactgaccgaccttttttgcccaagatcaacaacagttacggctgtcaaaactcacatcttctgaatgggctcctcaaggacgaactcggctttcaggggttcgtcctcagcgattggcaagcgcagcatgctggtgctgccactgccgttgctggacttgacatggccatgcccggtgacactcgcttcaacaccggagtcgccttctggggcgctaaccttaccaatgccattttgaacggcaccgttcccgaatatcggctcgatgacatggccatgcgtattatggcggcctttttcaaagttggaaagaccctggacgatgttcctgacatcaacttctcgtcttggacaaaagacaccatcggcccgctgcactgggcggcccaggacaatgtgcaggtcatcaaccaacacgttgatgtccgtcaagaccacggcgccctcattcgcaccatcgctgcccgcggtactgtcttactaaaaaatgagggatcactgcctctgaacaagccgaaatttgttgctgtcattggtgaagatgctggccctcgtcctgttggtcccaatggctgccctgatcagggttgcaataacggcactctggctgctggatggggatctggcaccgccagtttcccttatctcatcactcctgatagtgctcttcagtttcaagccgtttcggatggctcgcgatacgaaagcatcctcagcaactgggattatgagcgcacagaggccttggtttcccaggcggatgctactgctctggttttcgtcaatgcaaactctggcgaaggatatatcagcgttgatggaaacgaaggtgatcgcaagaacctcactctctggaatggaggagacgagcttattcaacgagtcgctgcggccaacaacaacaccatcgtcatcatccattcggttggtcccgttctagtcactgactggtacgagaatcccaatatcacggctatcatctgggccggcttacccggacaggagtctggcaactctatcgccgatattctttacggccgcgtgaaccctggtggcaagacacctttcacctggggtccaactgttgagagctacggcgttgacgtcctgagagagcccaacaatggcaatggtgctccccagagcgatttcgacgagggagtcttcatcgattaccgttggtttgaccggcagtcgggtgttgataacaatgcatcagcgccgaggaacagcagcagcagccacgccccaatcttcgagtttggctatggcctttcgtacacaacctttgaattctccaatcttcagattgagaggcatgacgttcacgattacgtccctaccactgggcagacgagccctgcgccgagatttggtgctaactacagtacgaactacgacgactacgtctttcccgagggcgaaatccgttacatctatcaacacatctacccatacctcaattcctcagacccaaaggaggcattggctgatcctaaatacggccaaactgcagaagagttcctcccagagggcgctcttgatgcctcaccgcagcctaggctcccagcttctggagggcccggaggcaacccaatgctttgggacgtcatattcacggtcaccgcgaccgtgaccaacacgggtaaggttgctggggacgaagtggcacagctttacgtttctcttggtggacctgacgatccgattcgagtcctccgtgggttcgaccgcattcacatcgcgcctggagcctcgcaaaccttccgtgcggaactcacgcgccgggacctcagcaactgggatgttgtcacgcaaaattggttcatcagccagtacgaaaagacggtctttgtcgggagctcatcccgaaacctccctctcagcactcgcctcgaatag

SEQ ID NO: 76 - protein, 890 aa, Verticillium dahliae
MKLTLATALLAASGCVSAGQPKLKHPQRQTNSSSELAFSPPHYPSPWMNPQATGWEDAYARAREVVEQMTLLEKVNLTTGVGWSGDLCVGNVGSIPRIGWRGLCLQDGPQGIRFADYVSYFTSSQTAGATWDRGLLYQRAHAIGAEGVAKGVDVVLGPAIGPLGRLPAGGRNWEGFAVDPYLSGVAVAESVRGIQDAGAIANVKHYIVNEQEHFRQAGEAQGYGYDVDEALSSNVDDKTMHELYLWPFADAVRAGAGSVMCSYQQINNSYGCQNSHLLNGLLKDELGFQGFVLSDWQAQHAGAATAVAGLDMAMPGDTRFNTGVAFWGANLTNAILNGTVPEYRLDDMAMRIMAAFFKVGKTLDDVPDINFSSWTKDTIGPLHWAAQDNVQVINQHVDVRQDHGALIRTIAARGTVLLKNEGSLPLNKPKFVAVIGEDAGPRPVGPNGCPDQGCNNGTLAAGWGSGTASFPYLITPDSALQFQAVSDGSRYESILSNWDYERTEALVSQADATALVFVNANSGEGYISVDGNEGDRKNLTLWNGGDELIQRVAAANNNTIVIIHSVGPVLVTDWYENPNITAIIWAGLPGQESGNSIADILYGRVNPGGKTPFTWGPTVESYGVDVLREPNNGNGAPQSDFDEGVFIDYRWFDRQSGVDNNASAPRNSSSSHAPIFEFGYGLSYTTFEFSNLQIERHDVHDYVPTTGQTSPAPRFGANYSTNYDDYVFPEGEIRYIYQHIYPYLNSSDPKEALADPKYGQTAEEFLPEGALDASPQPRLPASGGPGGNPMLWDVIFTVTATVTNTGKVAGDEVAQLYVSLGGPDDPIRVLRGFDRIHIAPGASQTFRAELTRRDLSNWDVVTQNWFISQYEKTVFVGSSSRNLPLSTRLE

SEQ ID NO: 77 - DNA, 2418 bp, Podospora anserina
atgaaactcaataagccattcctggccatttatttggctttcaacttggccgaggcttcgaaaactccggattgcatcagtggtccgctggcaaagaccttggcatgtgatacaacggcgtcacctcctgcgcgagcagctgctcttgtgcaggctttaaatatcacggaaaagcttgtgaatctagtggagtatgtcaagtcaagagaagctcctttagggatttcaattcagctaatcactcctcatagcatgagcctcggtgcagaaaggatcggccttccagcttatgcttggtggaacgaagctcttcatggtgttgccgcgtcgcctggggtctccttcaatcaggccggacaagaattctcacacgctacttcatttgcgaatactattacgctagcagccgcctttgacaatgacctggtttacgaggtggcggataccatcagcactgaagcgcgagcgttcagcaatgccgagctcgctggactggattactggacgcctaacatcaacccgtacaaagatccgagatgggggaggggccatgaggtttgttaccttagccttcttttccgtgccgtgcagttgctgagaactcaaaagacacccggagaagatccggtacacatcaaaggctacgtccaagcacttctcgagggtctagaagggagagacaagatcagaaaggtgattgccacttgtaaacactttgcagcctatgatttggagagatggcaaggggctcttagatacaggttcaatgctgttgtgacctcgcaggatctttcggagtactacctccaaccgtttcaacaatgcgctcgagacagcaaggtcgggtctttcatgtgctcatataatgcgctcaacggaacaccggcatgtgcaagcacgtatttgatggacgacatccttcgaaaacactggaattggaccgagcacaacaactatataacgagcgactgtaatgctattcaggacttcctccccaactttcacaacttcagccaaactccagctcaagccgccgctgatgcttataacgccggtacagacaccgtctgtgaggtgcctggataccccccactcacagatgtaatcggagcatacaatcagtctctgctgtcagaggaaattatcgaccgagcacttcgcagattatacgaaggcctcatccgagctggctatctcgactcagcctccccacatccatacaccaaaatctcatggtcccaagtaaacacccccaaagcccaagccctggctctccagtccgccaccgacgggatagtccttctcaaaaacaacggcctccttcccctagacctcaccaacaaaaccatagccctcataggccactgggccaatgcaacccgccaaatgctaggcggctacagcggtatccccccttactacgccaacccaatctatgcagccacccagctcaacgtcacttttcatcacgccccaggaccggtgaaccagtcatctccctccacaaatgacacctggacctcccccgccctctccgcggcttccaaatcggatatcatcctctacctcggcggcaccgacctctccatcgcagccgaagaccgagacagagactccatcgcctggccatccgctcaactttccttgttaacctccctcgcccagatgggaaaacccacaatcgtagcaagactaggcgaccaagtagacgacacccccctgctctccaacccaaacatctcctccatcctatgggtaggctacccaggccaatcaggcggaacagccctcttgaacatcatcaccggagtcagctcccccgccgctcgactgcccgtcacagtctacccagaaacttacacctccctcatccccctgacagccatgtccctccgcccaacctccgcccgcccaggccggacttacaggtggtacccctcccccgtgctccccttcggccacggcctccactacacaacctttaccgccaaattcggcgtctttgagtccctcaccatcaacattgccgaactcgtttccaactgtaacgaacgatacctcgacctctgccggttcccgcaggtgtccgtctgggtgtcgaatacgggagaactcaaatctgactatgtcgcccttgtttttgtcaggggtgagtacggaccggagccgtacccgatcaagacgctggtggggtacaagcggataagggatatcgagccggggactacgggggcggcgccggtgggggtggtggtgggggatttggctagggtggatttgggggggaatagggttttgtttccggggaagtatgagtttctgctggatgtggagggggggagggatagggttgtgatcgagttggttggggaggaggtggtgttggagaagttccctcagccgcctgcggcgggttga

SEQ ID NO: 78 - protein, 805 aa, Podospora anserina
MKLNKPFLAIYLAFNLAEASKTPDCISGPLAKTLACDTTASPPARAAALVQALNITEKLVNLVEYVKSREAPLGISIQLITPHSMSLGAERIGLPAYAWWNEALHGVAASPGVSFNQAGQEFSHATSFANTITLAAAFDNDLVYEVADTISTEARAFSNAELAGLDYWTPNINPYKDPRWGRGHEVCYLSLLFRAVQLLRTQKTPGEDPVHIKGYVQALLEGLEGRDKIRKVIATCKHFAAYDLERWQGALRYRFNAVVTSQDLSEYYLQPFQQCARDSKVGSFMCSYNALNGTPACASTYLMDDILRKHWNWTEHNNYITSDCNAIQDFLPNFHNFSQTPAQAAADAYNAGTDTVCEVPGYPPLTDVIGAYNQSLLSEEIIDRALRRLYEGLIRAGYLDSASPHPYTKISWSQVNTPKAQALALQSATDGIVLLKNNGLLPLDLTNKTIALIGHWANATRQMLGGYSGIPPYYANPIYAATQLNVTFHHAPGPVNQSSPSTNDTWTSPALSAASKSDIILYLGGTDLSIAAEDRDRDSIAWPSAQLSLLTSLAQMGKPTIVARLGDQVDDTPLLSNPNISSILWVGYPGQSGGTALLNIITGVSSPAARLPVTVYPETYTSLIPLTAMSLRPTSARPGRTYRWYPSPVLPFGHGLHYTTFTAKFGVFESLTINIAELVSNCNERYLDLCRFPQVSVWVSNTGELKSDYVALVFVRGEYGPEPYPIKTLVGYKRIRDIEPGTTGAAPVGVVVGDLARVDLGGNRVLFPGKYEFLLDVEGGRDRVVIELVGEEVVLEKFPQPPAAG

SEQ ID NO: 79 - protein, 721 aa, Thermotoga neapolitana
MEKVNEILSQLTLEEKVKLVVGVGLPGLFGNPHSRVAGAAGETHPVPRVGLPAFVLADGPAGLRINPTRENDENTYYTTAFPVEIMLASTWNRELLEEVGKAMGEEVREYGVDVLLAPAMNIHRNPLCGRNFEYYSEDPVLSGEMASSFVKGVQSQGVGACIKHFVANNQETNRMVVDTIVSERALREIYLRGFEIAVKKSKPWSVMSAYNKLNGKYCSQNEWLLKKVLREEWGFEGFVMSDWYAGDNPVEQLKAGNDLIMPGKAYQVNTERRDEIEEIMEALKEGKLSEEVLDECVRNILKVLVNAPSFKNYRYSNKPDLEKHAKVAYEAGAEGVVLLRNEEALPLSENSKIALFGTGQIETIKGGTGSGDTHPRYAISILEGIKERGLNFDEELAKTYEDYIKKMRETEEYKPRRDSWGTIIKPKLPENFLSEKEIHKLAKKNDVAVIVISRISGEGYDRKPVKGDFYLSDDETDLIKTVSREFHEQGKKVIVLLNIGSPVEVVSWRDLVDGILLVWQAGQETGRIVADVLTGRINPSGKLPTTFPRDYSDVPSWTFPGEPKDNPQKVVYEEDIYVGYRYYDTFGVEPAYEFGYGLSYTTFEYSDLNVSFDGETLRVQYRIENTGGRAGKEVSQVYIKAPKGKIDKPFQELKAFHKTRLLNPGESEEVVLEIPVRDLASFNGEEWVVEAGEYEVRVGASSRNIKLKGTFSVGEERRFKP

SEQ ID NO: 80 - protein, 249 aa, Trichoderma reesei
MKSCAILAALGCLAGSVLGHGQVQNFTINGQYNQGFILDYYYQKQNTGHFPNVAGWYAEDLDLGFISPDQYTTPDIVCHKNAAPGAISATAAAGSNIVFQWGPGVWPHPYGPIVTYVVECSGSCTTVNKNNLRWVKIQEAGINYNTQVWAQQDLINQGNKWTVKIPSSLRPGNYVFRHELLAAHGASSANGMQNYPQCVNIAVTGSGTKALPAGTPATQLYKPTDPGILFNPYTTITSYTIPGPALWQG

SEQ ID NO: 81 - protein, 226 aa, Thielavia terrestris
MLANGAIVFLAAALGVSGHYTWPRVNDGADWQQVRKADNWQDNGYVGDVTSPQIRCFQATPSPAPSVLNTTAGSTVTYWANPDVYHPGPVQFYMARVPDGEDINSWNGDGAVWFKVYEDHPTFGAQLTWPSTGKSSFAVPIPPCIKSGYYLLRAEQIGLHVAQSVGGAQFYISCAQLSVTGGGSTEPPNKVAFPGAYSATDPGILINIYYPVPTSYQNPGPAVFSC

SEQ ID NO: 82 - protein, 471 aa, Trichoderma reesei
MIVGILTTLATLATLAASVPLEERQACSSVWGQCGGQNWSGPTCCASGSTCVYSNDYYSQCLPGAASSSSSTRAASTTSRVSPTTSRSSSATPPPGSTTTRVPPVGSGTATYSGNPFVGVTPWANAYYASEVSSLAIPSLTGAMATAAAAVAKVPSFMWLDTLDKTPLMEQTLADIRTANKNGGNYAGQFVVYDLPDRDCAALASNGEYSIADGGVAKYKNYIDTIRQIVVEYSDIRTLLVIEPDSLANLVTNLGTPKCANAQSAYLECINYAVTQLNLPNVAMYLDAGHAGWLGWPANQDPAAQLFANVYKNASSPRALRGLATNVANYNGWNITSPPSYTQGNAVYNEKLYIHAIGPLLANHGWSNAFFITDQGRSGKQPTGQQQWGDWCNVIGTGFGIRPSANTGDSLLDSFVWVKPGGECDGTSDSSAPRFDSHCALPDALQPAPQAGAWFQAYFVQLLTNANPSFL

SEQ ID NO: 83 - protein, 513 aa, Trichoderma reesei
MYRKLAVISAFLATARAQSACTLQSETHPPLTWQKCSSGGTCTQQTGSVVIDANWRWTHATNSSTNCYDGNTWSSTLCPDNETCAKNCCLDGAAYASTYGVTTSGNSLSIGFVTQSAQKNVGARLYLMASDTTYQEFTLLGNEFSFDVDVSQLPCGLNGALYFVSMDADGGVSKYPTNTAGAKYGTGYCDSQCPRDLKFINGQANVEGWEPSSNNANTGIGGHGSCCSEMDIWEANSISEALTPHPCTTVGQEICEGDGCGGTYSDNRYGGTCDPDGCDWNPYRLGNTSFYGPGSSFTLDTTKKLTVVTQFETSGAINRYYVQNGVTFQQPNAELGSYSGNELNDDYCTAEEAEFGGSSFSDKGGLTQFKKATSGGMVLVMSLWDDYYANMLWLDSTYPTNETSSTPGAVRGSCSTSSGVPAQVESQSPNAKVTFSNIKFGPIGSTGNPSGGNPPGGNRGTTTTRRPATTTGSSPGPTQSHYGQCGGIGYSGPTVCASGTTCQVLNPYYSQCL

SEQ ID NO: 84 - protein, 19 aa, Artificial Sequence
  synthetic GH61 Family Endoglucanase motif
  misc_feature (1)..(1): Xaa can be Ile, Leu, Met or Val
  misc_feature (3)..(6): Xaa can be any naturally occurring amino acid
  misc_feature (8)..(8): Xaa can be any naturally occurring amino acid
  misc_feature (10)..(10): Xaa can be Ile, Leu, Met or Val
  misc_feature (11)..(11): Xaa can be any naturally occurring amino acid
  misc_feature (13)..(13): Xaa can be any naturally occurring amino acid
  misc_feature (14)..(14): Xaa can be Glu or Gln
  misc_feature (15)..(18): Xaa can be any naturally occurring amino acid
  misc_feature (19)..(19): Xaa can be His, Asn or Gln
XPXXXXGXYXXRXXXXXXX

SEQ ID NO: 85 - protein, 20 aa, Artificial Sequence
  synthetic GH61 Family Endoglucanase motif
  misc_feature (1)..(1): Xaa can be Ile, Leu, Met or Val
  misc_feature (3)..(7): Xaa can be any naturally occurring amino acid
  misc_feature (9)..(9): Xaa can be any naturally occurring amino acid
  misc_feature (11)..(11): Xaa can be Ile, Leu, Met or Val
  misc_feature (12)..(12): Xaa can be any naturally occurring amino acid
  misc_feature (14)..(14): Xaa can be any naturally occurring amino acid
  misc_feature (15)..(15): Xaa can be Glu or Gln
  misc_feature (16)..(19): Xaa can be any naturally occurring amino acid
  misc_feature (20)..(20): Xaa can be His, Asn or Gln
XPXXXXXGXYXXRXXXXXXX

SEQ ID NO: 86 - protein, 19 aa, Artificial Sequence
  synthetic GH61 Family Endoglucanase motif
  misc_feature (1)..(1): Xaa can be Ile, Leu, Met or Val
  misc_feature (3)..(6): Xaa can be any naturally occurring amino acid
  misc_feature (8)..(8): Xaa can be any naturally occurring amino acid
  misc_feature (10)..(10): Xaa can be Ile, Leu, Met or Val
  misc_feature (11)..(11): Xaa can be any naturally occurring amino acid
  misc_feature (13)..(13): Xaa can be any naturally occurring amino acid
  misc_feature (14)..(14): Xaa can be Glu or Gln
  misc_feature (15)..(17): Xaa can be any naturally occurring amino acid
  misc_feature (19)..(19): Xaa can be His, Asn or Gln
XPXXXXGXYXXRXXXXXAX

SEQ ID NO: 87 - protein, 20 aa, Artificial Sequence
  synthetic GH61 Family Endoglucanase motif
  misc_feature (1)..(1): Xaa can be Ile, Leu, Met or Val
  misc_feature (3)..(7): Xaa can be any naturally occurring amino acid
  misc_feature (9)..(9): Xaa can be any naturally occurring amino acid
  misc_feature (11)..(11): Xaa can be Ile, Leu, Met or Val
  misc_feature (12)..(12): Xaa can be any naturally occurring amino acid
  misc_feature (14)..(14): Xaa can be any naturally occurring amino acid
  misc_feature (15)..(15): Xaa can be Glu or Gln
  misc_feature (16)..(18): Xaa can be any naturally occurring amino acid
  misc_feature (20)..(20): Xaa can be His, Asn or Gln
XPXXXXXGXYXXRXXXXXAX

SEQ ID NO: 88 - protein, 4 aa, Artificial Sequence
  synthetic GH61 Family Endoglucanase motif
  misc_feature (1)..(1): Xaa can be Phe or Trp
  misc_feature (2)..(2): Xaa can be Phe or Thr
  misc_feature (4)..(4): Xaa can be Ala, Ile or Val
XXKX

SEQ ID NO: 89 - protein, 10 aa, Artificial Sequence
  synthetic GH61 Family Endoglucanase motif
  misc_feature (2)..(3): Xaa can be any naturally occurring amino acid
  misc_feature (6)..(8): Xaa can be any naturally occurring amino acid
  misc_feature (9)..(9): Xaa can be Tyr or Trp
  misc_feature (10)..(10): Xaa can be Ala, Ile, Leu, Met or Val
HXXGPXXXXX

SEQ ID NO: 90 - protein, 9 aa, Artificial Sequence
  synthetic GH61 Family Endoglucanase motif
  misc_feature (2)..(2): Xaa can be any naturally occurring amino acid
  misc_feature (5)..(7): Xaa can be any naturally occurring amino acid
  misc_feature (8)..(8): Xaa can be Tyr or Trp
  misc_feature (9)..(9): Xaa can be Ala, Ile, Leu, Met or Val
HXGPXXXXX

SEQ ID NO: 91 - protein, 11 aa, Artificial Sequence
  synthetic GH61 Family Endoglucanase motif
  misc_feature (1)..(1): Xaa can be Glu or Gln
  misc_feature (2)..(2): Xaa can be any naturally occurring amino acid
  misc_feature (4)..(5): Xaa can be any naturally occurring amino acid
  misc_feature (7)..(7): Xaa can be any naturally occurring amino acid
  misc_feature (8)..(8): Xaa can be Glu, His, Gln or Asn
  misc_feature (9)..(9): Xaa can be Phe, Ile, Leu or Val
  misc_feature (10)..(10): Xaa can be any naturally occurring amino acid
  misc_feature (11)..(11): Xaa can be Ile, Leu or Val
XXYXXCXXXXX

SEQ ID NO: 92 - DNA, 3193 bp, Artificial Sequence
  synthetic Fv3C/Bgl3 chimeric beta-glucosidase
atgaagctgaattgggtcgccgcagccctgtctataggtgctgctggcactgacagcgcagttgctcttgcttctgcagttccagacactttggctggtgtaaaggtcagttttttttcaccatttcctcgtctaatctcagccttgttgccatatcgcccttgttcgctcggacgccacgcaccagatcgcgatcatttcctcccttgcagccttggttcctcttacgatcttccctccgcaattatcagcgcccttagtctacacaaaaacccccgagacagtctttcattgagtttgtcgacatcaagttgcttctcaactgtgcatttgcgtggctgtctacttctgcctctagacaaccaaatctgggcgcaattgaccgctcaaaccttgttcaaataaccttttttattcgagacgcacatttataaatatgcgcctttcaataataccgactttatgcgcggcggctgctgtggcggttgatcagaaagctgacgctcaaaaggttgtcacgagagatacactcgcatactcgccgcctcattatccttcaccatggatggaccctaatgctgttggctgggaggaagcttacgccaaagccaagagctttgtgtcccaactcactctcatggaaaaggtcaacttgaccactggtgttgggtaagcagctccttgcaaacagggtatctcaatcccctcagctaacaacttctcagatggcaaggcgaacgctgtgtaggaaacgtgggatcaattcctcgtctcggtatgcgaggtctctgtctccaggatggtcctcttggaattcgtctgtccgactacaacagcgcttttcccgctggcaccacagctggtgcttcttggagcaagtctctctggtatgagagaggtctcctgatgggcactgagttcaaggagaagggtatcgatatcgctcttggtcctgctactggacctcttggtcgcactgctgctggtggacgaaactgggaaggcttcaccgttgatccttatatggctggccacgccatggccgaggccgtcaagggtattcaagacgcaggtgtcattgcttgtgctaagcattacatcgcaaacgagcagggtaagccacttggacgatttgaggaattgacagagaactgaccctcttgtagagcacttccgacagagtggcgaggtccagtcccgcaagtacaacatctccgagtctctctcctccaacctggatgacaagactatgcacgagctctacgcctggcccttcgctgacgccgtccgcgccggcgtcggttccgtcatgtgctcgtacaaccagatcaacaactcgtacggttgccagaactccaagctcctcaacggtatcctcaaggacgagatgggcttccagggtttcgtcatgagcgattgggcggcccagcataccggtgccgcttctgccgtcgctggtctcgatatgagcatgcctggtgacactgccttcgacagcggatacagcttctggggcggaaacttgactctggctgtcatcaacggaactgttcccgcctggcgagttgatgacatggctctgcgaatcatgtctgccttcttcaaggttggaaagacgatagaggatcttcccgacatcaacttctcctcctggacccgcgacaccttcggcttcgtgcatacatttgctcaagagaaccgcgagcaggtcaactttggagtcaacgtccagcacgaccacaagagccacatccgtgaggccgctgccaagggaagcgtcgtgctcaagaacaccgggtcccttcccctcaagaacccaaagttcctcgctgtcattggtgaggacgccggtcccaaccctgctggacccaatggttgtggtgaccgtggttgcgataatggtaccctggctatggcttggggctcgggaacttcccaattcccttacttgatcacccccgatcaagggctctctaatcgagctactcaagacggaactcgatatgagagcatcttgaccaacaacgaatgggcttcagtacaagctcttgtcagccagcctaacgtgaccgctatcgttttcgccaatgccgactctggtgagggatacattgaagtcgacggaaactttggtgatcgcaagaacctcaccctctggcagcagggagacgagctcatcaagaacgtgtcgtccatatgccccaacaccattgtagttctgcacaccgtcggccctgtcctactcgccgactacgagaagaaccccaacatcactgccatcgtctgggctggtcttcccggccaagagtcaggcaatgccatcgctgatctcctctacggcaaggtcagccctggccgatctcccttcacttggggccgcacccgcgagagctacggtactgaggttctttatgaggcgaacaacggccgtggcgctcctcaggatgacttctctgagggtgtcttcatcgactaccgtcacttcgaccgacgatctccaagcaccgatggaaagagctctcccaacaacaccgctgctcctctctacgagttcggtcacggtctatcttggtcgacgttcaagttctccaacctccacatccagaagaacaatgtcggccccatgagcccgcccaacggcaagacgattgcggctccctctctgggcagcttcagcaagaaccttaaggactatggcttccccaagaacgttcgccgcatcaaggagtttatctacccctacctgagcaccactacctctggcaaggaggcgtcgggtgacgctcactacggccagactgcgaaggagttcctccccgccggtgccctggacggcagccctcagcctcgctctgcggcctctggcgaacccggcggcaaccgccagctgtacgacattctctacaccgtgacggccaccattaccaacacgggctcggtcatggacgacgccgttccccagctgtacctgagccacggcggtcccaacgagccgcccaaggtgctgcgtggcttcgaccgcatcgagcgcattgctcccggccagagcgtcacgttcaaggcagacctgacgcgccgtgacctgtccaactgggacacgaagaagcagcagtgggtcattaccgactaccccaagactgtgtacgtgggcagctcctcgcgcgacctgccgctgagcgcccgcctgccatga

SEQ ID NO: 93 - protein, 898 aa, Artificial Sequence
  synthetic Fv3C/Bgl3 chimeric beta-glucosidase
MKLNWVAAALSIGAAGTDSAVALASAVPDTLAGVKKADAQKVVTRDTLAYSPPHYPSPWMDPNAVGWEEAYAKAKSFVSQLTLMEKVNLTTGVGWQGERCVGNVGSIPRLGMRGLCLQDGPLGIRLSDYNSAFPAGTTAGASWSKSLWYERGLLMGTEFKEKGIDIALGPATGPLGRTAAGGRNWEGFTVDPYMAGHAMAEAVKGIQDAGVIACAKHYIANEQEHFRQSGEVQSRKYNISESLSSNLDDKTMHELYAWPFADAVRAGVGSVMCSYNQINNSYGCQNSKLLNGILKDEMGFQGFVMSDWAAQHTGAASAVAGLDMSMPGDTAFDSGYSFWGGNLTLAVINGTVPAWRVDDMALRIMSAFFKVGKTIEDLPDINFSSWTRDTFGFVHTFAQENREQVNFGVNVQHDHKSHIREAAAKGSVVLKNTGSLPLKNPKFLAVIGEDAGPNPAGPNGCGDRGCDNGTLAMAWGSGTSQFPYLITPDQGLSNRATQDGTRYESILTNNEWASVQALVSQPNVTAIVFANADSGEGYIEVDGNFGDRKNLTLWQQGDELIKNVSSICPNTIVVLHTVGPVLLADYEKNPNITAIVWAGLPGQESGNAIADLLYGKVSPGRSPFTWGRTRESYGTEVLYEANNGRGAPQDDFSEGVFIDYRHFDRRSPSTDGKSSPNNTAAPLYEFGHGLSWSTFKFSNLHIQKNNVGPMSPPNGKTIAAPSLGSFSKNLKDYGFPKNVRRIKEFIYPYLSTTTSGKEASGDAHYGQTAKEFLPAGALDGSPQPRSAASGEPGGNRQLYDILYTVTATITNTGSVMDDAVPQLYLSHGGPNEPPKVLRGFDRIERIAPGQSVTFKADLTRRDLSNWDTKKQQWVITDYPKTVYVGSSSRDLPLSARLP

SEQ ID NO: 94 - DNA, 3157 bp, Artificial Sequence
  synthetic Fv3C/Te3A/Bgl3 chimeric beta- glucosidase
atgaagctgaattgggtcgccgcagccctgtctataggtgctgctggcactgacagcgcagttgctcttgcttctgcagttccagacactttggctggtgtaaaggtcagttttttttcaccatttcctcgtctaatctcagccttgttgccatatcgcccttgttcgctcggacgccacgcaccagatcgcgatcatttcctcccttgcagccttggttcctcttacgatcttccctccgcaattatcagcgcccttagtctacacaaaaacccccgagacagtctttcattgagtttgtcgacatcaagttgcttctcaactgtgcatttgcgtggctgtctacttctgcctctagacaaccaaatctgggcgcaattgaccgctcaaaccttgttcaaataaccttttttattcgagacgcacatttataaatatgcgcctttcaataataccgactttatgcgcggcggctgctgtggcggttgatcagaaagctgacgctcaaaaggttgtcacgagagatacactcgcatactcgccgcctcattatccttcaccatggatggaccctaatgctgttggctgggaggaagcttacgccaaagccaagagctttgtgtcccaactcactctcatggaaaaggtcaacttgaccactggtgttgggtaagcagctccttgcaaacagggtatctcaatcccctcagctaacaacttctcagatggcaaggcgaacgctgtgtaggaaacgtgggatcaattcctcgtctcggtatgcgaggtctctgtctccaggatggtcctcttggaattcgtctgtccgactacaacagcgcttttcccgctggcaccacagctggtgcttcttggagcaagtctctctggtatgagagaggtctcctgatgggcactgagttcaaggagaagggtatcgatatcgctcttggtcctgctactggacctcttggtcgcactgctgctggtggacgaaactgggaaggcttcaccgttgatccttatatggctggccacgccatggccgaggccgtcaagggtattcaagacgcaggtgtcattgcttgtgctaagcattacatcgcaaacgagcagggtaagccacttggacgatttgaggaattgacagagaactgaccctcttgtagagcacttccgacagagtggcgaggtccagtcccgcaagtacaacatctccgagtctctctcctccaacctggatgacaagactatgcacgagctctacgcctggcccttcgctgacgccgtccgcgccggcgtcggttccgtcatgtgctcgtacaaccagatcaacaactcgtacggttgccagaactccaagctcctcaacggtatcctcaaggacgagatgggcttccagggtttcgtcatgagcgattgggcggcccagcataccggtgccgcttctgccgtcgctggtctcgatatgagcatgcctggtgacactgccttcgacagcggatacagcttctggggcggaaacttgactctggctgtcatcaacggaactgttcccgcctggcgagttgatgacatggctctgcgaatcatgtctgccttcttcaaggttggaaagacgatagaggatcttcccgacatcaacttctcctcctggacccgcgacaccttcggcttcgtgcatacatttgctcaagagaaccgcgagcaggtcaactttggagtcaacgtccagcacgaccacaagagccacatccgtgaggccgctgccaagggaagcgtcgtgctcaagaacaccgggtcccttcccctcaagaacccaaagttcctcgctgtcattggtgaggacgccggtcccaaccctgctggacccaatggttgtggtgaccgtggttgcgataatggtaccctggctatggcttggggctcgggaacttcccaattcccttacttgatcacccccgatcaagggctctctaatcgagctactcaagacggaactcgatatgagagcatcttgaccaacaacgaatgggcttcagtacaagctcttgtcagccagcctaacgtgaccgctatcgttttcgccaatgccgactctggtgagggatacattgaagtcgacggaaactttggtgatcgcaagaacctcaccctctggcagcagggagacgagctcatcaagaacgtgtcgtccatatgccccaacaccattgtagttctgcacaccgtcggccctgtcctactcgccgactacgagaagaaccccaacatcactgccatcgtctgggctggtcttcccggccaagagtcaggcaatgccatcgctgatctcctctacggcaaggtcagccctggccgatctcccttcacttggggccgcacccgcgagagctacggtactgaggttctttatgaggcgaacaacggccgtggcgctcctcaggatgacttctctgagggtgtcttcatcgactaccgtcacttcgacaagtacaacatcacgcctatctacgagttcggtcacggtctatcttggtcgacgttcaagttctccaacctccacatccagaagaacaatgtcggccccatgagcccgcccaacggcaagacgattgcggctccctctctgggcaacttcagcaagaaccttaaggactatggcttccccaagaacgttcgccgcatcaaggagtttatctacccctacctgaacaccactacctctggcaaggaggcgtcgggtgacgctcactacggccagactgcgaaggagttcctccccgccggtgccctggacggcagccctcagcctcgctctgcggcctctggcgaacccggcggcaaccgccagctgtacgacattctctacaccgtgacggccaccattaccaacacgggctcggtcatggacgacgccgttccccagctgtacctgagccacggcggtcccaacgagccgcccaaggtgctgcgtggcttcgaccgcatcgagcgcattgctcccggccagagcgtcacgttcaaggcagacctgacgcgccgtgacctgtccaactgggacacgaagaagcagcagtgggtcattaccgactaccccaagactgtgtacgtgggcagctcctcgcgcgacctgccgctgagcgcccgcctgccatga

SEQ ID NO: 95 - protein, 886 aa, Artificial Sequence
  synthetic Fv3C/Te3A/Bgl3 chimeric beta- glucosidase
MKLNWVAAALSIGAAGTDSAVALASAVPDTLAGVKKADAQKVVTRDTLAYSPPHYPSPWMDPNAVGWEEAYAKAKSFVSQLTLMEKVNLTTGVGWQGERCVGNVGSIPRLGMRGLCLQDGPLGIRLSDYNSAFPAGTTAGASWSKSLWYERGLLMGTEFKEKGIDIALGPATGPLGRTAAGGRNWEGFTVDPYMAGHAMAEAVKGIQDAGVIACAKHYIANEQEHFRQSGEVQSRKYNISESLSSNLDDKTMHELYAWPFADAVRAGVGSVMCSYNQINNSYGCQNSKLLNGILKDEMGFQGFVMSDWAAQHTGAASAVAGLDMSMPGDTAFDSGYSFWGGNLTLAVINGTVPAWRVDDMALRIMSAFFKVGKTIEDLPDINFSSWTRDTFGFVHTFAQENREQVNFGVNVQHDHKSHIREAAAKGSVVLKNTGSLPLKNPKFLAVIGEDAGPNPAGPNGCGDRGCDNGTLAMAWGSGTSQFPYLITPDQGLSNRATQDGTRYESILTNNEWASVQALVSQPNVTAIVFANADSGEGYIEVDGNFGDRKNLTLWQQGDELIKNVSSICPNTIVVLHTVGPVLLADYEKNPNITAIVWAGLPGQESGNAIADLLYGKVSPGRSPFTWGRTRESYGTEVLYEANNGRGAPQDDFSEGVFIDYRHFDKYNITPIYEFGHGLSWSTFKFSNLHIQKNNVGPMSPPNGKTIAAPSLGNFSKNLKDYGFPKNVRRIKEFIYPYLNTTTSGKEASGDAHYGQTAKEFLPAGALDGSPQPRSAASGEPGGNRQLYDILYTVTATITNTGSVMDDAVPQLYLSHGGPNEPPKVLRGFDRIERIAPGQSVTFKADLTRRDLSNWDTKKQQWVITDYPKTVYVGSSSRDLPLSARLP

SEQ ID NO: 96 - protein, 23 aa, Artificial Sequence
  synthetic chimeric beta-glucosidase motif
  misc_feature (2)..(2): Xaa can be any naturally occurring amino acid
  misc_feature (6)..(6): Xaa can be any naturally occurring amino acid
  misc_feature (15)..(15): Xaa can be any naturally occurring amino acid
  misc_feature (17)..(17): Xaa can be any naturally occurring amino acid
  misc_feature (21)..(21): Xaa can be any naturally occurring amino acid
AXSPPXYPSPWMDPXAXGWEXAY

SEQ ID NO: 97 - protein, 32 aa, Artificial Sequence
  synthetic chimeric beta-glucosidase motif
  misc_feature (3)..(3): Xaa can be any naturally occurring amino acid
  misc_feature (7)..(8): Xaa can be any naturally occurring amino acid
  misc_feature (11)..(11): Xaa can be any naturally occurring amino acid
  misc_feature (23)..(23): Xaa can be any naturally occurring amino acid
  misc_feature (26)..(26): Xaa can be any naturally occurring amino acid
AKXFVSXXTLXEKVNLTTGVGWXGEXCVGNVG

SEQ ID NO: 98 - protein, 18 aa, Artificial Sequence
  synthetic chimeric beta-glucosidase motif
  misc_feature (3)..(3): Xaa can be any naturally occurring amino acid
  misc_feature (7)..(7): Xaa can be any naturally occurring amino acid
  misc_feature (10)..(10): Xaa can be any naturally occurring amino acid
  misc_feature (17)..(17): Xaa can be any naturally occurring amino acid
PRXGMRXLCXQDGPLGXR

SEQ ID NO: 99 - protein, 16 aa, Artificial Sequence
  synthetic chimeric beta-glucosidase motif
  misc_feature (6)..(7): Xaa can be any naturally occurring amino acid
  misc_feature (9)..(9): Xaa can be any naturally occurring amino acid
  misc_feature (12)..(12): Xaa can be any naturally occurring amino acid
YNSAFXXGXTAXASWS

SEQ ID NO: 100 - protein, 19 aa, Artificial Sequence
  synthetic chimeric beta-glucosidase motif
  misc_feature (2)..(2): Xaa can be any naturally occurring amino acid
  misc_feature (9)..(11): Xaa can be any naturally occurring amino acid
  misc_feature (17)..(17): Xaa can be any naturally occurring amino acid
GXIACAKHXXXNEQEHXRQ

SEQ ID NO: 101 - protein, 27 aa, Artificial Sequence
  synthetic chimeric beta-glucosidase motif
  misc_feature (5)..(5): Xaa can be any naturally occurring amino acid
  misc_feature (10)..(10): Xaa can be any naturally occurring amino acid
  misc_feature (13)..(13): Xaa can be any naturally occurring amino acid
  misc_feature (15)..(15): Xaa can be any naturally occurring amino acid
  misc_feature (19)..(19): Xaa can be any naturally occurring amino acid
  misc_feature (23)..(23): Xaa can be any naturally occurring amino acid
LSSNXDDKTXHEXYXWPFXDAVXAGVG

SEQ ID NO: 102 - protein, 21 aa, Artificial Sequence
  synthetic chimeric beta-glucosidase motif
  misc_feature (5)..(5): Xaa can be any naturally occurring amino acid
  misc_feature (7)..(7): Xaa can be any naturally occurring amino acid
  misc_feature (12)..(12): Xaa can be any naturally occurring amino acid
  misc_feature (19)..(19): Xaa can be any naturally occurring amino acid
MCSYXQXNNSYXCQNSKLXNG

SEQ ID NO: 103 - protein, 32 aa, Artificial Sequence
  synthetic chimeric beta-glucosidase motif
  misc_feature (11)..(11): Xaa can be any naturally occurring amino acid
  misc_feature (15)..(15): Xaa can be any naturally occurring amino acid
  misc_feature (17)..(17): Xaa can be any naturally occurring amino acid
  misc_feature (19)..(19): Xaa can be any naturally occurring amino acid
  misc_feature (27)..(27): Xaa can be any naturally occurring amino acid
GFQGFVMSDWXAQHXGXAXAVAGLDMXMPGDT

SEQ ID NO: 104 - protein, 19 aa, Artificial Sequence
  synthetic chimeric beta-glucosidase motif
  misc_feature (7)..(7): Xaa can be any naturally occurring amino acid
  misc_feature (13)..(13): Xaa can be any naturally occurring amino acid
  misc_feature (16)..(16): Xaa can be any naturally occurring amino acid
NLTLAVXNGTVPXWRXDDM

SEQ ID NO: 105 - protein, 26 aa, Artificial Sequence
  synthetic chimeric beta-glucosidase motif
  misc_feature (2)..(2): Xaa can be any naturally occurring amino acid
  misc_feature (5)..(5): Xaa can be any naturally occurring amino acid
  misc_feature (7)..(7): Xaa can be any naturally occurring amino acid
  misc_feature (13)..(13): Xaa can be any naturally occurring amino acid
  misc_feature (22)..(22): Xaa can be any naturally occurring amino acid
PXFLXVXGEDAGXNPAGPNGCXDRGC

SEQ ID NO: 106 - protein, 16 aa, Artificial Sequence
  synthetic chimeric beta-glucosidase motif
  misc_feature (6)..(6): Xaa can be any naturally occurring amino acid
  misc_feature (12)..(12): Xaa can be any naturally occurring amino acid
GTLAMXWGSGTXFPYL

SEQ ID NO: 107 - protein, 29 aa, Artificial Sequence
  synthetic chimeric beta-glucosidase motif
  misc_feature (7)..(8): Xaa can be any naturally occurring amino acid
  misc_feature (15)..(15): Xaa can be any naturally occurring amino acid
  misc_feature (20)..(20): Xaa can be any naturally occurring amino acid
AIVFANXXSGEGYIXVDGNXGDRKNLTLW

SEQ ID NO: 108 - protein, 17 aa, Artificial Sequence
  synthetic chimeric beta-glucosidase motif
  misc_feature (2)..(2): Xaa can be any naturally occurring amino acid
  misc_feature (7)..(7): Xaa can be any naturally occurring amino acid
  misc_feature (12)..(12): Xaa can be any naturally occurring amino acid
DXLYGKXSPGRXPFTWG

SEQ ID NO: 109 - protein, 19 aa, Artificial Sequence
  synthetic chimeric beta-glucosidase motif
  misc_feature (2)..(2): Xaa can be any naturally occurring amino acid
  misc_feature (7)..(7): Xaa can be any naturally occurring amino acid
  misc_feature (12)..(12): Xaa can be any naturally occurring amino acid
  misc_feature (15)..(16): Xaa can be any naturally occurring amino acid
  misc_feature (18)..(18): Xaa can be any naturally occurring amino acid
PXYEFGXGLSWXTFXXSXL

SEQ ID NO: 110 - protein, 7 aa, Artificial Sequence
  synthetic chimeric beta-glucosidase motif
  misc_feature (2)..(2): Xaa can be any naturally occurring amino acid
  misc_feature (5)..(5): Xaa can be any naturally occurring amino acid
LXDYXFP

SEQ ID NO: 111 - protein, 15 aa, Artificial Sequence
  synthetic chimeric beta-glucosidase motif
  misc_feature (5)..(6): Xaa can be any naturally occurring amino acid
  misc_feature (9)..(9): Xaa can be any naturally occurring amino acid
  misc_feature (12)..(12): Xaa can be any naturally occurring amino acid
EFLPXXALXGSXQPR

SEQ ID NO: 112 - protein, 12 aa, Artificial Sequence
  synthetic chimeric beta-glucosidase motif
  misc_feature (3)..(3): Xaa can be any naturally occurring amino acid
  misc_feature (8)..(9): Xaa can be any naturally occurring amino acid
  misc_feature (11)..(11): Xaa can be any naturally occurring amino acid
SGXPGGNXXLXD

SEQ ID NO: 113 - protein, 11 aa, Artificial Sequence
  synthetic chimeric beta-glucosidase motif
  misc_feature (4)..(4): Xaa can be any naturally occurring amino acid
  misc_feature (6)..(6): Xaa can be any naturally occurring amino acid
YTVXAXITNTG

SEQ ID NO: 114 - protein, 16 aa, Artificial Sequence
  synthetic chimeric beta-glucosidase motif
  misc_feature (6)..(6): Xaa can be any naturally occurring amino acid
  misc_feature (8)..(8): Xaa can be any naturally occurring amino acid
  misc_feature (10)..(10): Xaa can be any naturally occurring amino acid
  misc_feature (15)..(15): Xaa can be any naturally occurring amino acid
VLRGFXRXEXIAPGXS

SEQ ID NO: 115 - protein, 19 aa, Artificial Sequence
  synthetic chimeric beta-glucosidase motif
  misc_feature (10)..(12): Xaa can be any naturally occurring amino acid
  misc_feature (14)..(14): Xaa can be any naturally occurring amino acid
TRRDLSNWDXXXQXWVITD

SEQ ID NO: 116 - protein, 14 aa, Artificial Sequence
  synthetic chimeric beta-glucosidase motif
  misc_feature (7)..(7): Xaa can be any naturally occurring amino acid
  misc_feature (11)..(11): Xaa can be any naturally occurring amino acid
  misc_feature (13)..(13): Xaa can be any naturally occurring amino acid
VGSSSRXLPLXAXL

SEQ ID NO: 117 - protein, 17 aa, Trichoderma reesei
MYRKLAVISAFLATARA

SEQ ID NO: 118 - DNA, 28 bp, Artificial Sequence
  synthetic primer
caccatgagatatagaacagctgccgct

SEQ ID NO: 119 - DNA, 40 bp, Artificial Sequence
  synthetic primer
cgaccgccctgcggagtcttgcccagtggtcccgcgacag

SEQ ID NO: 120 - DNA, 40 bp, Artificial Sequence
  synthetic primer
ctgtcgcgggaccactgggcaagactccgcagggcggtcg

SEQ ID NO: 121 - DNA, 20 bp, Artificial Sequence
  synthetic primer
cctacgctaccgacagagtg

SEQ ID NO: 122 - DNA, 20 bp, Artificial Sequence
  synthetic primer
gtctagactggaaacgcaac

SEQ ID NO: 123 - DNA, 21 bp, Artificial Sequence
  synthetic primer
gagttgtgaagtcggtaatcc

SEQ ID NO: 124 - DNA, 35 bp, Artificial Sequence
  synthetic primer
caccatgaaagcaaacgtcatcttgtgcctcctgg

SEQ ID NO: 125 - DNA, 43 bp, Artificial Sequence
  synthetic primer
ctattgtaagatgccaacaatgctgttatatgccggcttgggg

SEQ ID NO: 126 - DNA, 21 bp, Artificial Sequence
  synthetic primer
gagttgtgaagtcggtaatcc

SEQ ID NO: 127 - DNA, 18 bp, Artificial Sequence
  synthetic primer
cacgaagagcggcgattc

SEQ ID NO: 128 - DNA, 23 bp, Artificial Sequence
  synthetic primer
cacccatgctgctcaatcttcag

SEQ ID NO: 129 - DNA, 23 bp, Artificial Sequence
  synthetic primer
ttacgcagacttggggtcttgag

SEQ ID NO: 130 - DNA, 20 bp, Artificial Sequence
  synthetic primer
gcttgagtgtatcgtgtaag

SEQ ID NO: 131 - DNA, 21 bp, Artificial Sequence
  synthetic primer
gcaacggcaaagccccacttc

SEQ ID NO: 132 - DNA, 32 bp, Artificial Sequence
  synthetic primer
gtagcggccgcctcatctcatctcatccatcc

SEQ ID NO: 133 - DNA, 24 bp, Artificial Sequence
  synthetic primer
caccatgcagctcaagtttctgtc

SEQ ID NO: 134 - DNA, 32 bp, Artificial Sequence
  synthetic primer
ggttactagtcaactgcccgttctgtagcgag

SEQ ID NO: 135 - DNA, 29 bp, Artificial Sequence
  synthetic primer
catgcgatcgcgacgttttggtcaggtcg

SEQ ID NO: 136 - DNA, 40 bp, Artificial Sequence
  synthetic primer
gacagaaacttgagctgcatggtgtgggacaacaagaagg

SEQ ID NO: 137 - DNA, 29 bp, Artificial Sequence
  synthetic primer
caccatggttcgcttcagttcaatcctag

SEQ ID NO: 138 - DNA, 22 bp, Artificial Sequence
  synthetic primer
gtggctagaagatatccaacac

SEQ ID NO: 139 - DNA, 29 bp, Artificial Sequence
  synthetic primer
catgcgatcgcgacgttttggtcaggtcg

SEQ ID NO: 140 - DNA, 39 bp, Artificial Sequence
  synthetic primer
gaactgaagcgaaccatggtgtgggacaacaagaaggac

SEQ ID NO: 141 - DNA, 21 bp, Artificial Sequence
  synthetic primer
gtagttatgcgcatgctagac

SEQ ID NO: 142 - DNA, 22 bp, Artificial Sequence
  synthetic primer
gtggctagaagatatccaacac

SEQ ID NO: 143 - DNA, 21 bp, Artificial Sequence
  synthetic primer
gtagttatgcgcatgctagac

SEQ ID NO: 144 - DNA, 28 bp, Artificial Sequence
  synthetic primer
ccggctcagtatcaaccactaagcacat

SEQ ID NO: 145 - DNA, 24 bp, Artificial Sequence
  synthetic primer
caccatgaagctgaattgggtcgc

SEQ ID NO: 146 - DNA, 19 bp, Artificial Sequence
  synthetic primer
ttactccaacttggcgctg

SEQ ID NO: 147 - DNA, 20 bp, Artificial Sequence
  synthetic primer
aagccaagagctttgtgtcc

SEQ ID NO: 148 - DNA, 20 bp, Artificial Sequence
  synthetic primer
tatgcacgagctctacgcct

SEQ ID NO: 149 - DNA, 20 bp, Artificial Sequence
  synthetic primer
atggtaccctggctatggct

SEQ ID NO: 150 - DNA, 20 bp, Artificial Sequence
  synthetic primer
cggtcacggtctatcttggt

SEQ ID NO: 151 - DNA, 21 bp, Artificial Sequence
  synthetic primer
gtagttatgcgcatgctagac

SEQ ID NO: 152 - DNA, 22 bp, Artificial Sequence
  synthetic primer
gtggctagaagatatccaacac

SEQ ID NO: 153 - DNA, 20 bp, Artificial Sequence
  synthetic primer
cgtctaactcgaacatctgc

SEQ ID NO: 154 - DNA, 32 bp, Artificial Sequence; identical to SEQ ID NO: 132
  synthetic primer

SEQ ID NO: 155 - DNA, 21 bp, Artificial Sequence
  synthetic primer
gtagttatgcgcatgctagac

SEQ ID NO: 156 - DNA, 18 bp, Artificial Sequence
  synthetic primer
cacgaagagcggcgattc

SEQ ID NO: 157 - DNA, 21 bp, Artificial Sequence
  synthetic primer
gcaacggcaaagccccacttc

SEQ ID NO: 158 - DNA, 32 bp, Artificial Sequence; identical to SEQ ID NO: 132
  synthetic primer

SEQ ID NO: 159 - DNA, 20 bp, Artificial Sequence
  synthetic primer
cgtctaactcgaacatctgc

SEQ ID NO: 160 - DNA, 30 bp, Artificial Sequence
  synthetic primer
catggcgcgcccaactgcccgttctgtagc

SEQ ID NO: 161 - DNA, 32 bp, Artificial Sequence; identical to SEQ ID NO: 132
  synthetic primer

SEQ ID NO: 162 - DNA, 27 bp, Artificial Sequence
  synthetic primer
gtagttatgcgcatgctagactgctcc

SEQ ID NO: 163 - DNA, 23 bp, Artificial Sequence
  synthetic primer
gcaggccgcatctccagtgaaag

SEQ ID NO: 164 - DNA, 20 bp, Artificial Sequence
  synthetic primer
cgtctaactcgaacatctgc

SEQ ID NO: 165 - DNA, 23 bp, Artificial Sequence
  synthetic primer
gcaggccgcatctccagtgaaag

SEQ ID NO: 166 - DNA, 28 bp, Artificial Sequence
  synthetic primer
agttgtgaagtcggtaatcccgctgtat

SEQ ID NO: 167 - DNA, 24 bp, Artificial Sequence
  synthetic primer
tcgtagcatggcatggtcacttca

SEQ ID NO: 168 - DNA, 35 bp, Artificial Sequence; identical to SEQ ID NO: 124
  synthetic primer

SEQ ID NO: 169 - DNA, 43 bp, Artificial Sequence; identical to SEQ ID NO: 125
  synthetic primer

SEQ ID NO: 170 - DNA, 40 bp, Artificial Sequence
  synthetic primer
agatcaccctctgtgtattgcaccatgaaagcaaacgtca

SEQ ID NO: 171 - DNA, 40 bp, Artificial Sequence
  synthetic primer
tgacgtttgctttcatggtgcaatacacagagggtgatct

SEQ ID NO: 172 - DNA, 21 bp, Artificial Sequence
  synthetic primer
gagttgtgaagtcggtaatcc

SEQ ID NO: 173 - DNA, 18 bp, Artificial Sequence
  synthetic primer
cacgaagagcggcgattc

SEQ ID NO: 174 - DNA, 25 bp, Artificial Sequence
  synthetic primer
caccatgatccagaagctttccaac

SEQ ID NO: 175 - DNA, 21 bp, Artificial Sequence
  synthetic primer
ctagttaaggcactgggcgta

SEQ ID NO: 176 - DNA, 29 bp, Artificial Sequence
  synthetic primer
catgcgatcgcgacgttttggtcaggtcg

SEQ ID NO: 177 - DNA, 41 bp, Artificial Sequence
  synthetic primer
gttggaaagcttctggatcatggtgtgggacaacaagaagg

SEQ ID NO: 178 - DNA, 25 bp, Artificial Sequence
  synthetic primer
caccatgatccagaagctttccaac

SEQ ID NO: 179 - DNA, 21 bp, Artificial Sequence
  synthetic primer
gctcagtatcaaccactaagc

SEQ ID NO: 180 - DNA, 21 bp, Artificial Sequence
  synthetic primer
gtagttatgcgcatgctagac

SEQ ID NO: 181 - DNA, 27 bp, Artificial Sequence
  synthetic primer
gtagttatgcgcatgctagactgctcc

SEQ ID NO: 182 - DNA, 23 bp, Artificial Sequence
  synthetic primer
gcaggccgcatctccagtgaaag

SEQ ID NO: 183 - DNA, 45 bp, Artificial Sequence
  synthetic primer
gctagcatggatgttttcccagtcacgacgttgtaaaacgacggc

SEQ ID NO: 184 - DNA, 53 bp, Artificial Sequence
  synthetic primer
ggaggttggagaacttgaacgtcgaccaagatagaccgtgaccgaactcgtag

SEQ ID NO: 185 - DNA, 43 bp, Artificial Sequence
  synthetic primer
tgccaggaaacagctatgaccatgtaatacgactcactatagg

SEQ ID NO: 186 - DNA, 53 bp, Artificial Sequence
  synthetic primer
ctacgagttcggtcacggtctatcttggtcgacgttcaagttctccaacctcc

SEQ ID NO: 187 - DNA, 42 bp, Artificial Sequence
  synthetic primer
taagctcgggccccaaataatgattttattttgactgatagt

SEQ ID NO: 188 - DNA, 45 bp, Artificial Sequence
  synthetic primer
gggatatcagctggatggcaaataatgattttattttgactgata

SEQ ID NO: 189 - DNA, 27 bp, Artificial Sequence
  synthetic primer
cggaatgagctagtaggcaaagtcagc

SEQ ID NO: 190 - DNA, 70 bp, Artificial Sequence
  synthetic primer
ctccttgatgcggcgaacgttcttggggaagccatagtccttaaggttcttgctgaagttgcccagagag

SEQ ID NO: 191 - DNA, 65 bp, Artificial Sequence
  synthetic primer
ggcttccccaagaacgttcgccgcatcaaggagtttatctacccctacctgaacaccactacctc

SEQ ID NO: 192 - DNA, 27 bp, Artificial Sequence
  synthetic primer
gatacacgaagagcggcgattctacgg

SEQ ID NO: 193 - DNA, 45 bp, Artificial Sequence; identical to SEQ ID NO: 183
  synthetic primer

SEQ ID NO: 194 - DNA, 71 bp, Artificial Sequence
  synthetic primer
gatagaccgtgaccgaactcgtagataggcgtgatgttgtacttgtcgaagtgacggtagtcgatgaagac

SEQ ID NO: 195 - DNA, 71 bp, Artificial Sequence
  synthetic primer
gtcttcatcgactaccgtcacttcgacaagtacaacatcacgcctatctacgagttcggtcacggtctatc

SEQ ID NO: 196 - DNA, 43 bp, Artificial Sequence; identical to SEQ ID NO: 185
  synthetic primer

SEQ ID NO: 197 - protein, 8 aa, Artificial Sequence
  synthetic hybrid/chimera beta-glucanase motif
YPSPWMDP

SEQ ID NO: 198 - protein, 11 aa, Artificial Sequence
  synthetic hybrid/chimera beta-glucanase motif
EKVNLTTGVGW

SEQ ID NO: 199 - protein, 5 aa, Artificial Sequence
  synthetic hybrid/chimera beta-glucanase motif
  misc_feature (3)..(3): Xaa can be Ile or Val
  misc_feature (5)..(5): Xaa can be Ile or Val
KGXDX

SEQ ID NO: 200 - protein, 9 aa, Artificial Sequence
  synthetic hybrid/chimera beta-glucanase motif
  misc_feature (7)..(7): Xaa can be any naturally occurring amino acid
CQNSKLXNG

SEQ ID NO: 201 - protein, 14 aa, Artificial Sequence
  synthetic hybrid/chimera beta-glucanase motif
  misc_feature (7)..(7): Xaa can be Leu, Ile or Val
  misc_feature (10)..(10): Xaa can be Ser or Thr
  misc_feature (11)..(11): Xaa can be Ile or Val
  misc_feature (13)..(13): Xaa can be any naturally occurring amino acid
NLTLAVXNGXXPXW

SEQ ID NO: 202 - protein, 8 aa, Artificial Sequence
  synthetic hybrid/chimera beta-glucanase motif
  misc_feature (3)..(3): Xaa can be Ser or Thr
  misc_feature (4)..(4): Xaa can be any naturally occurring amino acid
  misc_feature (7)..(7): Xaa can be Phe or Tyr
SWXXDTXG

SEQ ID NO: 203 - protein, 15 aa, Artificial Sequence
  synthetic hybrid/chimera beta-glucanase motif
  misc_feature (5)..(6): Xaa can be any naturally occurring amino acid
  misc_feature (9)..(9): Xaa can be any naturally occurring amino acid
  misc_feature (12)..(12): Xaa can be any naturally occurring amino acid
EFLPXXALXGSXQPR

SEQ ID NO: 204 - protein, 7 aa, Artificial Sequence
  synthetic loop sequence
FDRRSPG

SEQ ID NO: 205 - protein, 7 aa, Artificial Sequence
  synthetic loop sequence
  misc_feature (3)..(3): Xaa can be any naturally occurring amino acid
FDXYNIT

SEQ ID NO: 206 - protein, 250 aa, Thermoascus aurantiacus
MSFSKIIATAGVLASASLVAGHGFVQNIVIDGKKYYGGYLVNQYPYMSNPPEVIAWSTTATDLGFVDGTGYQTPDIICHRGAKPGALTAPVSPGGTVELQWTPWPDSHHGPVINYLAPCNGDCSTVDKTQLEFFKIAESGLINDDNPPGIWASDNLIAANNSWTVTIPTTIAPGNYVLRHEIIALHSAQNQDGAQNYPQCINLQVTGGGSDNPAGTLGTALYHDTDPGILINIYQKLSSYIIPGPPLYTG

SEQ ID NO: 207 - protein, 354 aa, Thermoascus aurantiacus
MSFSKIAAITGAITYASLAAAHGYVTGIVADGTYYGGYIVTQYPYMSTPPDVIAWSTKATDLGFVDPSSYASSDIICHKGAEPGALSAKVAAGGTVELQWTDWPESHKGPVIDYLAACNGDCSTVDKTKLEFFKIDESGLIDGSSAPGTWASDNLIANNNSWTVTIPSTIAPGNYVLRHEIIALHSAGNTNGAQNYPQCINLEVTGSGTDTPAGTLGTELYKATDPGILVNIYQTLTSYDIPGPALYTGGSSGSSGSSNTAKATTSTASSSIVTPTPVNNPTVTQTAVVDVTQTVSQNAAVATTTPASTAVATAVPTGTTFSFDSMTSDEFVSLMRATVNWLLSNKKHARDLSY

SEQ ID NO: 208 - protein, 884 aa, Nectria haematococca
MRFTVLLAAFSGLVPMVGSQADQKPLQLGVNNNTLAHSPPHYPSPWMDPAAPGWEEAYLKAKDFVSQLTLLEKVNLTTGVGWMGERCVGNVGSLPRFGMRGLCMQDGPLGIRLSDYNSAFPTGITAGASWSRALWYQRGLLMGTEHREKGIDVALGPATGPLGRTPTGGRNWEGFSVDPYVAGVAMAETVSGIQDGGTIACAKHYIGNEQEHHRQAPESIGRGYNITESLSSNVDDKTLHELYLWPFADAVKAGVGAIMCSYQQLNNSYGCQNSKLLNGILKDELGFQGFVMSDWQAQHAGAATAVAGLDMTMPGDTLFNTGYSFWGGNLTLAVVNGTVPDWRIDDMAMRIMAAFFKVGKTVEDLPDINFSSWSRDTFGYVQAAAQENWEQINFGVDVRHDHSEHIRLSAAKGTVLLKNSGSLPLKKPKFLAVVGEDAGPNPAGPNGCNDRGCNNGTLAMSWGSGTAQFPYLVTPDSALQNQAVLDGTRYESVLRNNQWEQTRSLISQPNVTAIVFANANSGEGYIDVDGNEGDRKNLTLWNEGDDLIKNVSSICPNTIVVLHTVGPVILTEWYDNPNITAIVWAGVPGQESGNALVDILYGKTSPGRSPFTWGRTRKSYGTDVLYEPNNGQGAPQDDFTEGVFIDYRHFDQVSPSTDGSKSNDESSPIYEFGHGLSWTTFEYSELNIQAHNKIPFDPPIGETIAAPVLGNYSTDLADYTFPDGIRYIYQFIYPWLNTSSSGREASGDPDYGKTAEEFLPPGALDGSAQPRPPSSGAPGGNPHLWDVLYTVSAIITNTGNATSDEIPQLYVSLGGENEPVRVLRGFDRIENIAPGQSVRFTTDITRRDLSNWDVVSQNWVITDYEKTVYVGSSSRNLPLKATLK

SEQ ID NO: 209 - protein, 929 aa, Podospora anserina
MKFSVVVAAALASGALATPQYPPKLIKRDLPAGAYSPPVYPSPWMNPEADGWAEAYVKAREFVSQMTLLEKVNLTTGTGWAPAGSEQCVGQVGAIPRLGLRSLCMHDAPLGIRGTDPAGYNSAFPSGQTAAATWDRQLMYRRGYAIGKEAKGKGINVILGPVAGPLGRMPAGPAAGRNWEGFSPDPVLTGVGMAETVKGHQDAGVIACAKHFIGNEQEHFRQPAGVGEARGYGFNISETLSSNIDDKTMHELYLWPFADAVRAGAGSFMCSYPAGQQVNNSYGCQNSKLMNGLLKDELGFQGFVLSDWQAQHTGAAAAAAGLDMSPAGMPGDTEFNTGVSFWGTNLTVAVLNGTVPAYRIDDMAMRIMAAFFKVEPAGKSIELDPINFSFWSLDTYGPIHWAAGEGHQQINYHVDVRADHANLIREPAGIAAKGTVLLKNTGSLPLNKPKFVAVIGEDAGPNPNGPNSCAPAGDRGCNNGTLAMGWGSGTANFPYLITPPAGDAALQAQAIKDGSRYESILTNYPAGAASQTRALVSQDNVTAIVFVNADSGEGYINFEGNMGDRNNLTLWRGGPAGDDLVKNVSSWCSNTIVVIHSTGPVLISEWYDSPNITAILWAGLPGQPAGESGNSITDVLYGKVNPSGKSPFTWGATREGYGADVLYTPNNGEGAPAGPQQDFSEGVFIDYRYFDKANTSVIYEFGHGLSYTPAGTFEYSNIQVTKKNAGPYKPTTGQTAPAPTFGNFSTDLSDYLFPDEEFPYPAGVYQYIYPYLNTTDPRNASGDPHFGQTAEEFMPPHAIDDSPQPLLPSSPAGGKNSPGGNRALYDILYEVTADITNTGEIVGDEVVQLYVSLGGPDDPKPAGVVLRDFGKLRIEPGQTAKFRGLLTRRDLSNWDVVSQDWVISEHTKTVFVPAGGKSSRDLGLSAVLE

SEQ ID NO: 210 - protein, 61 aa, Fusarium verticillioides
MKLNWVAAALSIGAAGTDSAVALASAVPDTLAGVKKADAQKVVTRDTLAYSPPHYPSPWMD

SEQ ID NO: 211 - protein, 504 aa, Geobacillus stearothermophilus
MKVVNVPSNGREKFKKNWKFCVGTGRLGLALQKEYLDHLKLVQEKIGFRYIRGHGLLSDDVGIYREVEIDGEMKPFYNFTYIDRIVDSYLALNIRPFIEFGFMPKALASGDQTVFYWKGNVTPPKDYNKWRDLIVAVVSHFIERYGIEEVRTWLFEVWNEPNLVNFWKDANKQEYFKLYEVTARAVKSVDPHLQVGGPAICGGSDEWITDFLHFCAERRVPVDFVSRHAYTSKAPHKKTFEYYYQELELEPPEDMLEQFKTVRALIRQSPFPHLPLHITEYNTSYSPINPVHDTALNAAYIARILSEGGDYVDSFSYWTFSDVFEEMDVPKALFHGGFGLVALHSIPKPTFHAFTFFNALGDELLYRDGEMIVTRRKDGSIAAVLWNLVMEKGEGLTKEVQLVIPVSFSAVFIKRQIVNEQYGNAWRVWKQMGRPRFPSRQAVETLPSAQPHVMTEQRRATDGVIHLSIVLSKNEVTLIEIEQVRDETSTYVGLDDGEITSYSS

SEQ ID NO: 212 - protein, 497 aa, Thermoanaerobacter saccharolyticum
MIKVRVPDFSDKKFSDRWRYCVGTGRLGLALQKEYIETLKYVKENIDFKYIRGHGLLCDDVGIYVVGDEVKPFYNFTYIDRIFDSFLEIGIRPFVEIGFMPKKLASGTQTVFYWEGNVTPPKDYEKWSDLVKAVLHHFISRYGIEEVLKWPFEIWNEPNLKEFWKDADEKEYFKLYKVTAKAIKEVNENLKVGGPAICGGADYWIEDFLNFCYEENVPVDFVSRHATTSKQGEYTPHLIYQEIMPSEYMLNEFKTVREIIKNSHFPNLPFHITEYNTSYSPQNPVHDTPFNAAYIARILSEGGDYVDSFSYWTFSDVFEERDVPRSQFHGGFGLVALNMIPKPTFYTFKFFNAMGEEMLYRDEHMLVTRRDDGSVALIAWNEVMDKTENPDEDYEVEIPVRFRDVFIKRQLIDEEHGNPWGTWIHMGRPRYPSKEQVNTLREVAKPEIMTSQPVANDGYLNLKFKLGKNAVVLYELTERIDESSTYIGLDDSKINGY

SEQ ID NO: 213 - protein, 302 aa, Penicillium simplicissimum
QASVSIDAKFKAHGKKYLGTIGDQYTLTKNTKNPAIIKADFGQLTPENSMKWDATEPNRGQFTFSGSDYLVNFAQSNGKLIRGHTLVWHSQLPGWVSSITDKNTLISVLKNHITTVMTRYKGKIYAWDVLNEIFNEDGSLRNSVFYNVIGEDYVRIAFETARSVDPNAKLYINDYNLDSAGYSKVNGMVSHVKKWLAAGIPIDGIGSQTHLGAGAGSAVAGALNALASAGTKEIAITELDIAGASSTDYVNVVNACLNQAKCVGITVWGVADPDSWRSSSSPLLFDGNYNPKAAYNAIANAL

SEQ ID NO: 214 - protein, 329 aa, Thermoascus aurantiacus
MVRPTILLTSLLLAPFAAASPILEERQAAQSVDQLIKARGKVYFGVATDQNRLTTGKNAAIIQADFGQVTPENSMKWDATEPSQGNFNFAGADYLVNWAQQNGKLIRGHTLVWHSQLPSWVSSITDKNTLTNVMKNHITTLMTRYKGKIRAWDVVNEAFNEDGSLRQTVFLNVIGEDYIPIAFQTARAADPNAKLYINDYNLDSASYPKTQAIVNRVKQWRAAGVPIDGIGSQTHLSAGQGAGVLQALPLLASAGTPEVAITELDVAGASPTDYVNVVNACLNVQSCVGITVWGVADPDSWRASTTPLLFDGNFNPKPAYNAIVQDLQQ

SEQ ID NO: 215 - protein, 485 aa, Fusarium sp.
MNPLSLGLAALSLLGYVGVNFVAAFPTDSNSGSEVLISVNGHVKHQELDGFGASQAFQRAEDILGKDGLSKEGTQHVLDLLFSKDIGAGFSILRNGIGSSNSSDKNFMNSIEPFSPGSPGAKPHYVWDGYDSGQLTVAQEAFKRGLKFLYGDAWSAPGYMKTNHDENNGGYLCGVTGAACASGDWKQAYADYLLQWVEFYRKSGVKVTNLGFLNEPQFAAPYAGMLSNGTQAADFIRVLGKTIRKRGIHDLTIACCDGEGWDLQEDMMAGLTAGPDPAINYLSVVTGHGYVSPPNHPLSTTKKTWLTEWADLTGQFTPYTFYNNSGQGEGMTWAGRIQTALVDANVSGFLYWIGAENSTTNSALINMIGDKVIPSKRFWAFASFSRFARPGARRIEATSSVPLVTVSSFLNTDGTVATQVLNNDTVAHSVQLVVSGTGRNPHSLKPFLTDNSNDLTALKHLKATGKGSFQTTIPPRSLVSFVTDF

SEQ ID NO: 216 - protein, 598 aa, Trichoderma reesei
VVPPAGTPWGTAYDKAKAALAKLNLQDKVGIVSGVGWNGGPCVGNTSPASKISYPSLCLQDGPLGVRYSTGSTAFTPGVQAASTWDVNLIRERGQFIGEEVKASGIHVILGPVAGPLGKTPQGGRNWEGFGVDPYLTGIAMGQTINGIQSVGVQATAKHYILNEQELNRETISSNPDDRTLHELYTWPFADAVQANVASVMCSYNKVNTTWACEDQYTLQTVLKDQLGFPGYVMTDWNAQHTTVQSANSGLDMSMPGTDFNGNNRLWGPALTNAVNSNQVPTSRVDDMVTRILAAWYLTGQDQAGYPSFNISRNVQGNHKTNVRAIARDGIVLLKNDANILPLKKPASIAVVGSAAIIGNHARNSPSCNDKGCDDGALGMGWGSGAVNYPYFVAPYDAINTRASSQGTQVTLSNTDNTSSGASAARGKDVAIVFITADSGEGYITVEGNAGDRNNLDPWHNGNALVQAVAGANSNVIVVVHSVGAIILEQILALPQVKAVVWAGLPSQESGNALVDVLWGDVSPSGKLVYTIAKSPNDYNTRIVSGGSDSFSEGLFIDYKHFDDANITPRYEFGYGLSYTKFNYSRLSVLSTAKSGPA

(19) United States
(12) Patent Application Publication
(10) Pub. No.: US 2018/0163242 A1
(43) Pub. Date: Jun. 14, 2018

(54) GLYCOSYL HYDROLASE ENZYMES AND USES THEREOF FOR BIOMASS HYDROLYSIS

(71) Applicant: DANISCO US INC, Palo Alto, CA (US)

(72) Inventors: COLIN MITCHINSON, HALF MOON BAY, CA (US); MEREDITH K. FUJDALA, SAN JOSE, CA (US); WILLIAM D. HITZ, REHOBOTH BEACH, DE (US); MEGAN Y. HSI, SAN JOSE, CA (US); STEVEN S. KIM, FREMONT, CA (US); KEITH D. WING, WILMINGTON, DE (US)

(21) Appl. No.: 15/647,775

(22) Filed: Jul. 12, 2017

Related U.S. Application Data
(63) Continuation of application No. 14/004,881, filed on Nov. 20, 2013, now abandoned.
(60) Provisional application No. 61/453,931, filed on Mar. 17, 2011.

(51) Int. Cl. C12P 19/14; C12P 7/14; D21C 11/00; D21C 5/00; C12P 19/02; C12N 9/42; C12N 9/24

(52) U.S. Cl. CPC C12P 19/14; C12P 7/14; Y02E 50/16; D21C 11/0007; D21C 5/00; Y02E 50/17; C12N 9/2445; C12N 9/2437; C12Y 302/01032; C12Y 302/01021; C12N 9/2485; D21C 5/005; C12P 19/02

(57) ABSTRACT

The present invention relates to compositions that can be used in hydrolyzing biomass such as compositions comprising a polypeptide having glycosyl hydrolase (GH) family 61/endoglucanase activity and/or a β-glucosidase polypeptide, methods for hydrolyzing biomass material, and methods for using such compositions.

### CROSS REFERENCE TO RELATED APPLICATIONS

This application claims the benefit of U.S. Provisional Application No. 61/453,931, filed Mar. 17, 2011, which is hereby incorporated by reference in its entirety.

### SEQUENCE LISTING

The content of the electronically submitted sequence listing in ASCII text (file name: NB31566USPCN_SEQLIST; size: 419,201 bytes and date of creation Nov. 20, 2013) is incorporated herein by reference in its entirety.

### 1. TECHNICAL FIELD

The present disclosure generally pertains to glycosyl hydrolase enzymes, and engineered enzyme compositions, engineered fermentation broth compositions, and other compositions comprising such enzymes, and methods of making, or using in a research, industrial or commercial setting the enzymes and compositions, e.g., for saccharification or conversion of biomass materials comprising hemicellulose and optionally cellulose into fermentable sugars.

### 2. BACKGROUND

Bioconversion of renewable lignocellulosic biomass to a fermentable sugar that is subsequently fermented to produce alcohol (e.g., ethanol) as an alternative to liquid fuels has attracted the intensive attention of researchers since the oil crisis of the 1970s (Bungay, H. R., “Energy: the biomass options”. NY: Wiley; 1981; Olsson L, Hahn-Hagerdal B. Enzyme Microb Technol 1996,18:312-31; Zaldivar, J et al., Appl Microbiol Biotechnol 2001, 56: 17-34; Galbe, M et al., Appl Microbiol Biotechnol 2002, 59:618-28). Ethanol has been used as a 10% blend to gasoline in the USA or as a neat vehicle fuel in Brazil in the past decades. The importance of fuel bioethanol will increase with higher prices for oil and gradual depletion of its sources. Additionally, fermentable sugars are increasingly used to produce plastics, polymers and other bio-based materials. The demand for abundant low cost fermentable sugars, which can be used in lieu of petroleum-based fuel feedstock, grows rapidly. Chiefly among the useful renewable biomass materials are cellulose and hemicellulose (xylans), which can be converted into fermentable sugars. The enzymatic conversion of these polysaccharides to soluble sugars, e.g., glucose, xylose, arabinose, galactose, mannose, and/or other hexoses and pentoses, occurs due to combined actions of various enzymes. For example, endo-1,4-β-glucanases (EG) and exo-cellobiohydrolases (CBH) catalyze the hydrolysis of insoluble cellulose to cellooligosaccharides (e.g., with cellobiose being a main product), while β-glucosidases (BGL) convert the oligosaccharides to glucose. Xylanases together with other accessory proteins (non-limiting examples of which include L-α-arabinofuranosidases, feruloyl and acetylxylan esterases, glucuronidases, and β-xylosidases) catalyze the hydrolysis of hemicelluloses.

The cell walls of plants are composed of a heterogenous mixture of complex polysaccharides that interact through covalent and noncovalent means. Complex poly-saccharides of higher plant cell walls include, e.g., cellulose (β-1,4 glucan), which generally makes up 35-50% of carbon found in cell wall components. Cellulose polymers self associate through hydrogen bonding, van der Waals interactions and hydrophobic interactions to form semi-crystalline cellulose microfibrils. These microfibrils also include noncrystalline regions, generally known as amorphous cellulose. The cellulose microfibrils are embedded in a matrix formed of hemicelluloses (including, e.g., xylans, arabinans, and mannans), pectins (e.g., galacturonans and galactans), and various other β-1,3 and β-1,4 glucans. These polymers are often substituted with, e.g., arabinose, galactose and/or xylose residues to yield highly complex arabinoxylans, arabinogalactans, galactomannans, and xyloglucans. The hemicellulose matrix is, in turn, surrounded by polyphenolic lignin.

In order to obtain useful fermentable sugars from biomass materials, the lignin is typically permeabilized and the hemicellulose disrupted to allow access by the cellulose-hydrolyzing enzymes. A consortium of enzymatic activities may be necessary to break down the complex matrix of a biomass material before fermentable sugars can be obtained. Regardless of the type of cellulosic feedstock, the cost and hydrolytic efficiency of enzymes are major factors that restrict the commercialization of biomass bioconversion processes. Production costs of microbially produced enzymes are linked to the productivity of the enzyme-producing strain and the final activity yield from fermentation. The hydrolytic efficiency of a multienzyme complex can depend on a multitude of factors, e.g., properties of individual enzymes, the synergies among them, and their ratio in the multienzyme blend. There exists a need in the art to identify enzyme and/or enzymatic compositions that are capable of converting plant and/or other cellulosic or hemicellulosic materials into fermentable sugars with sufficient or improved efficacy, improved fermentable sugar yields, and/or improved capacity to act on a greater variety of cellulosic or hemicellulosic materials.

### 3. SUMMARY

The disclosure provides certain polypeptides having cellulase or celluloytic activity, including, e.g., certain β-glucosidase and endoglucanase polypeptides, and certain polypetpides having hemicellulolytic activity, including, e.g., xylanase (e.g., endoxylanase), xylosidase (e.g., β-xylosidase), arabinofuranosidase (e.g., L-α-arabinofuranosidase), that provide added benefits in saccharification of cellulosic and/or hemicellulosic biomass materials. The disclosure also provides nucleic acids encoding these polypeptides, recombinant cells expressing these nucleic acids, vectors and expression cassettes comprising these nucleic acids. Moreover, the disclosure provides methods of making and using the polypeptides and nucleic acids. The disclosure also provides compositions comprising a blend or mixture of 2 or more (e.g., 2 or more, 3 or more, 4 or more, 5 or more, etc.) enzymes selected from the polypeptides of the disclosure, and suitable ratios or relative weights of the polypeptides present in the composition to achieve saccharification or provide improved saccharification efficacy and/or efficiency. One or more or all of the enzymes of the disclosure can be heterologous to the host cell. On the other hand, one or more or all of the enzymes of the disclosure can be genetically engineered or modified such that they are expressed at a different level as they are in a corresponding wild type host cell. Moreover, the disclosure provides methods of use, in a research setting, an industrial setting (e.g., in the production of biofuels), or in a commercial setting.

For purpose of the present disclosure, enzyme can be referred to by the enzyme classes to which they are categorized by those skilled in the art. They are also referred to by their respective enzymatic activities. For example, a xylanase is referred to as a polypeptide having xylanase activity or, interchangeably, as a xylanase polypeptide. Accordingly, the disclosure is based, in part, on the discovery of certain novel enzymes and variants having xylanase activity, β-xylosidase activity, L-α-arabinofuranosidase activity, β-glucosidase activity, and/or endoglucanase activities. The disclosure is also based on the identification of novel enzyme compositions comprising certain particular blends or weight ratios of polypeptides having these hemicelluloytic activities and/or celluloytic activities, which allow for efficient saccharification of cellulosic and hemicellulosic materials.

The enzymes and/or enzyme compositions of the disclosure are used to produce fermentable sugars from biomass. The sugars can then be used by microorganisms for ethanol production, e.g., by fermentation or other culturing means, or can be used to produce other useful bio-products or bio-materials. The disclosure provides industrial applications (e.g., saccharification processes, ethanol production processes) using the enzymes and/or enzyme compositions described herein. Among their varied uses, the enzymes and/or enzyme compositions of the disclosure can advantageously reduce the cost of enzymes in a number of industrial processes, including, e.g., in biofuel production. Relatedly, the disclosure provides the use of the enzymes and/or the enzyme compositions of the invention in a commercial setting. For example, the enzymes and/or enzyme compositions of the disclosure can be sold in a suitable market place together with instructions for typical or preferred methods of using the enzymes and/or compositions. Accordingly the enzymes and/or enzyme compositions of the disclosure can be used or commercialized within a merchant enzyme supplier model, where the enzymes and/or enzyme compositions of the disclosure are sold to a manufacturer of bioethanol, a fuel refinery, or a biochemical or biomaterials manufacturer in the business of producing fuels or bio-products. In some aspects, the enzyme and/or enzyme composition of the disclosure can be marketed or commercialized using an on-site bio-refinery model, wherein the enzyme and/or enzyme composition is produced or prepared in a facility at or near to a fuel refinery or biochemical/biomaterial manufacturer's facility, and the enzyme and/or composition of the invention is tailored to the specific needs of the fuel refinery or biochemical/biomaterial manufacturer on a real-time basis. Moreover, the disclosure relates to providing these manufacturers with technical support and/or instructions for using the enzymes and.or enzyme compositions such that the desired bio-product (e.g., biofuel, bio-chemicals, bio-materials, etc) can be manufactured and marketed.

Accordingly, in a first aspect, the invention pertains to a number of polypeptides, including variants thereof, having glycosyl hydrolase activities. The invention pertains to isolated polypeptides, variants, and the nucleic acid encoding the polypeptides and variants. In some aspects, the disclosure provides isolated, synthetic or recombinant polypeptides comprising an amino acid sequence having at least about 60% (e.g., at least about 60%, 65%, 70%, 75%, 80%, 85%, 90%, 91%, 92%, 93%, 94%, 95%, 96%, 97%, 98%, 99%, or 100%) sequence identity to any one of SEQ ID NOs: 44, 54, 56, 58, 60, 62, 64, 66, 68, 70, 72, 74, 76, 78, 79, 93, and 95, over a region of at least about 10 (e.g., at least about 10, 15, 20, 25, 30, 35, 40, 45, 50, 55, 60, 65, 70, 75, 80, 85, 90, 95, 100, 125, 150, 175, 200, 225, 250, 275, 300) residues, or over the full length catalytic domain (CD) or the full length carbohydrate binding domain (CBM). In certain embodiments, the isolated, synthetic, or recombiant polypeptides have β-glucosidase activity. In certain embodiments, the isolated, synthetic, or recombinant polypeptides are β-glucosidase polypeptides, which include, e.g., variants, mutants, and fusion/hybrid/chimeric β-glucosidase polypeptides. For the instant disclosure, the terms “fusion,” “hybrid” and “chimeric” are used interchangeably and as equivalents to each other. In certain embodiments, the disclosure provides a polypeptide having β-glucosidase activity that is a hybrid or chimera of two or more β-glucosidase sequences. For example, the first of the two or more β-glucosidase sequences is at least about 200 (e.g., at least about 200, 250, 300, 350, 400, or 500) amino acid residues in length and comprises one or more or all of the amino acid sequence motifs of SEQ ID NOs: 96-108, In some embodiments, the second of the two or more β-glucosidase sequences is at least about 50 (e.g., at least about 50, 75, 100, 125, 150, 175, or 200) amino acid residues in length and comprises one or more or all of the amino acid sequence motifs of SEQ ID NOs: 109-116. In particular, the first of the two or more β-glucosidase sequences is one that is at least about 200 amino acid residues in length and comprises at least 2 (e.g., at least 2, 3, 4, or all) of the amino acid sequence motifs of SEQ ID NOs: 197-202, and the second of the two or more β-glucosidase is at least 50 amino acid residues in length and comprises SEQ ID NO:203. In some embodiments, the first sequence is located at the N-terminus, whereas the second sequence is located at the C-terminus of the chimeric or hybrid β-glucosidase polypeptide. In some embodiments, the first sequence is connected by its C-terminal residue to the second sequence by its N-terminal residue. For example, the first sequence is immediately adjacent or directly connected to the second sequence. In other embodiments, the first sequence is not immediately adjacent to the second sequence, but rather the first sequence is connected to the second sequence via a linker domain. In some embodiments, the first sequence, the second sequence, or both sequences, comprise 1 or more glycosylation sites. In some embodiments, the first or the second sequence comprises a loop sequence or a sequence encoding a loop-like structure. The loop sequence can be about 3, 4, 5, 6, 7, 8, 9, 10, or 11 amino acid residues in length, comprising a sequence of FDRRSPG (SEQ ID NO:204), or of FD(R/K)YNIT (SEQ ID NO:205). In other embodiments, the linker domain connecting the first and the second sequences comprises such a loop sequence. In some embodiments, the hybrid or chimeric β-glucosidase polypeptide has improved stability as compared to the counterpart β-glucosidase polypeptides from which each of the first, the second, or the linker domain sequences are derived. The improved stability is, e.g., an improved proteolytic stability, reflected in improved stability or resistance to proteolytic cleavage during storage under standard storage conditions, or during expression and/or production under standard expression/production conditions. For example, the hybrid/chimeric polypeptide is less susceptible to proteolytic cleavage at either a residue within the loop sequence or at a residue or position that is not within the loop sequence.

In certain embodiments, the disclosure provides an isolated, synthetic, or recombinant polypeptide having β-glucosidase activity, which is a hybrid of at least 2 (e.g., 2, 3, or even 4) β-glucosidase sequences, wherein the first of the at least 2 β-glucosidase sequences is at least about 200 (e.g., at least about 200, 250, 300, 350, or 400) amino acid residues in length and comprises a sequence that has at least about 60% (e.g., at least about 65%, 70%, 75%, 80%, 85%, 90%, 91%, 92%, 93%, 94%, 95%, 96%, 97%, 98%, 99%, or 100%) identity to a sequence of equal length of any one of SEQ ID NOs: 44, 54, 56, 58, 62, 64, 66, 68, 70, 72, 74, 76, 78, and 79, whereas the second of the at least 2 β-glucosidase sequences is at least about 50 (e.g., at least about 50, 75, 100, 125, 150, or 200) amino acid residues in length and comprises a sequence that has at least about 60% (e.g., at least about 65%, 70%, 75%, 80%, 85%, 90%, 91%, 92%, 93%, 94%, 95%, 96%, 97%, 98%, 99%, or 100%) identity to a sequence of equal length of SEQ ID NO:60. In an alternative embodiment, the disclosure provides an isolated, synthetic, or recombinant polypeptide encoding a polypeptide having β-glucosidase activity, which is a hybrid of at least 2 (e.g., 2, 3, or even 4) β-glucosidase sequences, wherein the first of the at least 2 β-glucosidase sequences is one that is at least about 200 (e.g., at least about 200, 250, 300, 350, or 400) amino acid residues in length and comprises a sequence that has at least about 60% (e.g., at least about 65%, 70%, 75%, 80%, 85%, 90%, 91%, 92%, 93%, 94%, 95%, 96%, 97%, 98%, 99%, or 100%) identity to a sequence of equal length of SEQ ID NO:60, whereas the second of the at least 2 β-glucosidase sequences is one that is at least about 50 (e.g., at least about 50, 75, 100, 125, 150, or 200) amino acid residues in length and comprises a sequence that has at least about 60% (e.g., at least about 65%, 70%, 75%, 80%, 85%, 90%, 91%, 92%, 93%, 94%, 95%, 96%, 97%, 98%, 99%, or 100%) identity to a sequence of equal length of any one of SEQ ID NOs: 44, 54, 56, 58, 62, 64, 66, 68, 70, 72, 74, 76, 78, and 79.

In particular, the first of the two or more β-glucosidase sequences is one that is at least about 200 amino acid residues in length and comprises at least 2 (e.g., at least 2, 3, 4, or all) of the amino acid sequence motifs of SEQ ID NOs: 197-202, and the second of the two or more β-glucosidase is at least 50 amino acid residues in length and comprises SEQ ID NO:203. In some embodiments, the first sequence is at the N-terminus, whereas the second sequence is at the C-termius of the chimeric or hybrid β-glucosidase polypeptide. In some embodiments, the first sequence is connected by its C-terminal residue to the second sequence by its N-terminal residue. For example, the first sequence is immediately adjacent or directly connected to the second sequence. In other embodiments, the first sequence is not immediately adjacent to the second sequence, but rather the first sequence is connected to the second sequence via a linker domain. The first sequence, the second sequence, or both sequences can comprise 1 or more glycosylation sites. In some embodiments, either the first or the second sequence comprises a loop sequence or a sequence that encodes a loop-like structure. In certain embodiments, the loop sequence is derived from a third β-glucosidase polypeptide, and is about 3, 4, 5, 6, 7, 8, 9, 10, or 11 amino acid residues in length, comprising a sequence of FDRRSPG (SEQ ID NO:204), or of FD(R/K)YNIT (SEQ ID NO:205). In certain embodiments, the linker domain connecting the first and the second sequences comprise such a loop sequence.

In an exemplary embodiment, the disclosure provides a hybrid or chimeric β-glucosidase polypeptide derived from two or more β-glucosidase sequences, wherein the first β-glucosidase sequence is derived from Fv3C and is at least about 200 amino acid residues in length, and the second β-glucosidase sequence is derived from a T. reesei Bgl3 (or “Tr3B”) polypeptide, and is at least about 50 amino acid residues in length. In some embodiments, the C-terminus of the first sequence is connected to the N-terminus of the second sequence.

Accordingly the first sequence is immediately adjacent or directly connected to the second sequence. In other embodiments, the first sequence is connected to the second sequence via a linker domain sequence. In some embodiments, either the first or the second sequence comprises a loop sequence. In some embodiments, the loop sequence is derived from a third β-glucosidase polypeptide. In certain embodiments, the loop sequence is about 3, 4, 5, 6, 7, 8, 9, 10, or 11 amino acid residues in length, comprising a sequence of FDRRSPG (SEQ ID NO:204), or of FD(R/K)YNIT (SEQ ID NO:205). In certain the linker domain sequence connecting the first and the second sequence comprises such a loop sequence. In certain embodiments, the loop sequence is derived from a Te3A polypeptide. In some embodiments, the hybrid or chimeric β-glucosidase polypeptide has improved stability over counterpart β-glucosidase polypeptides from which each of the chimeric parts are derived, e.g., over that of the Fv3C polypeptide, the Te3A polypeptide, and/or the Tr3B polypeptide. In some embodiments, the improved stability is an improved proteolytic stability, reflected in a reduced susceptibility to proteolytic cleavage at either a residue in the loop sequence or at a residue or position that is outside the loop sequence, during storage under standard storage conditions, or during expression and/or production, under standard expression/production conditions.

In certain aspects, the disclosure provides isolated, synthetic, or recombinant nucleotides encoding a β-glucosidase polypeptide having at least 60% (e.g., at least about 60%, 65%, 70%, 75%, 80%, 85%, 90%, 91%, 92%, 93%, 94%, 95%, 96%, 97%, 98%, 99%, or 100%) sequence identity to any one of SEQ ID NOs: 44, 54, 56, 58, 60, 62, 64, 66, 68, 70, 72, 74, 76, 78, 79, 93, and 95, over a region of at least about 10 (e.g., at least about 10, 15, 20, 25, 30, 35, 40, 45, 50, 55, 60, 65, 70, 75, 80, 85, 90, 95, 100, 125, 150, 175, 200, 225, 250, 275, 300) residues, or over the full length catalytic domain (CD) or the full length carbohydrate binding module (CBM). In some embodiments, the isolated, synthetic, or recombinant nucleotide encodes a β-glucosidase polypeptide that is a hybrid or chimera of two or more β-glucosidase sequences. In some embodiments, the hybrid/chimeric β-glucosidase polypeptide comprises a first sequence of at least about 200 (e.g., at least about 200, 250, 300, 350, 400, or 500) amino acid residues and comprises one or more or all of the amino acid sequence motifs of SEQ ID NOs: 96-108. In some embodiments, the hybrid/chimeric β-glucosidase polypeptide comprises a second β-glucosidase sequence that is at least about 50 (e.g., at least about 50, 75, 100, 125, 150, 175, or 200) amino acid residues and comprises one or more or all of the amino acid sequence motifs of SEQ ID NOs: 109-116. In particular, the first of the two or more β-glucosidase sequences is one that is at least about 200 amino acid residues in length and comprises at least 2 (e.g., at least 2, 3, 4, or all) of the amino acid sequence motifs of SEQ ID NOs: 197-202, and the second of the two or more β-glucosidase is at least 50 amino acid residues in length and comprises SEQ ID NO:203. In certain embodiments, the C-terminus of the first β-glucosidase sequence is connected to the N-terminus of the second β-glucosidase sequence. Alternatively, the first and the second β-glucosidase sequences are connected via a third nucleotide sequence encoding a linker domain. The first, second or the linker domain can comprise a loop sequence of about 3, 4, 5, 6, 7, 8, 9, 10, or 11 amino acid residues and having an amino acid sequence of FDRRSPG (SEQ ID NO:204), or of FD(R/K)YNIT (SEQ ID NO:205). In some embodiments, the loop sequence is derived from a third β-glucosidase polypeptide. In certain aspects,the disclosure provides an isolated, synthetic, or recombinant nucleotide encoding a polypeptide having β-glucosidase activity, which is a hybrid of at least 2 (e.g., 2, 3, or even 4) β-glucosidase sequences, wherein the first of the at least 2 β-glucosidase sequences is at least about 200 (e.g., at least about 200, 250, 300, 350, or 400) amino acid residues and comprises a sequence that has at least about 60% (e.g., at least about 65%, 70%, 75%, 80%, 85%, 90%, 91%, 92%, 93%, 94%, 95%, 96%, 97%, 98%, 99%, or 100%) identity to a sequence of equal length of any one of SEQ ID NOs: 44, 54, 56, 58, 62, 64, 66, 68, 70, 72, 74, 76, 78, and 79, whereas the second of the at least 2 β-glucosidase sequences is at least about 50 (e.g., at least about 50, 75, 100, 125, 150, or 200) amino acid residues and comprises a sequence that has at least about 60% (e.g., at least about 65%, 70%, 75%, 80%, 85%, 90%, 91%, 92%, 93%, 94%, 95%, 96%, 97%, 98%, 99%, or 100%) identity to a sequence of equal length of SEQ ID NO:60. Alternatively, the disclosure provides an isolated, synthetic, or recombinant nucleotide encoding a polypeptide having β-glucosidase activity, which is a hybrid of at least 2 (e.g., 2, 3, or even 4) β-glucosidase sequences, wherein the first of the at least 2 β-glucosidase sequences is at least about 200 (e.g., at least about 200, 250, 300, 350, or 400) amino acid residues in length and comprises a sequence that has at least about 60% (e.g., at least about 65%, 70%, 75%, 80%, 85%, 90%, 91%, 92%, 93%, 94%, 95%, 96%, 97%, 98%, 99%, or 100%) identity to a sequence of equal length of SEQ ID NO:60, whereas the second of the at least 2 β-glucosidase sequences is at least about 50 (e.g., at least about 50, 75, 100, 125, 150, or 200) amino acid residues in length and comprises a sequence that has at least about 60% (e.g., at least about 65%, 70%, 75%, 80%, 85%, 90%, 91%, 92%, 93%, 94%, 95%, 96%, 97%, 98%, 99%, or 100%) identity to a sequence of equal length of any one of SEQ ID NOs: 44, 54, 56, 58, 62, 64, 66, 68, 70, 72, 74, 76, 78, and 79. In particular, the first of the two or more β-glucosidase sequences is one that is at least about 200 amino acid residues in length and comprises at least 2 (e.g., at least 2, 3, 4, or all) of the amino acid sequence motifs of SEQ ID NOs: 197-202, and the second of the two or more β-glucosidase is at least 50 amino acid residues in length and comprises SEQ ID NO:203. In some embodiments, the nucleotide encodes a first amino acid sequence located at the N-terminus, and a second amino acid sequence, which is located at the C-terminus of the chimeric or hybrid β-glucosidase polypeptide. In some embodiments, the C-terminal residue of the first amino acid sequence is connected to the N-terminal residue of the second amino acid sequence. Alternatively, the first amino acid sequence is not immediately adjacent to the second amino acid sequence, but rather the first sequence is connected to the second sequence via a linker domain. In some embodiments, the first amino acid sequence, the second amino acid sequence, or the linker domain comprises an amino acid sequence that comprises a loop sequence, or a sequence that represents a loop-like structure, which is about 3, 4, 5, 6, 7, 8, 9, 10, or 11 amino acid residues in length, having an amino acid sequence of FDRRSPG (SEQ ID NO:204), or of FD(R/K)YNIT (SEQ ID NO:205). In certain embodiments, the loop sequence is derived from a third β-glucosidase polypeptide.

In some aspects, the disclosure provides isolated, synthetic, or recombinant nucleotides having at least 60% (e.g., at least about 60%, 65%, 70%, 75%, 80%, 85%, 90%, 91%, 92%, 93%, 94%, 95%, 96%, 97%, 98%, 99%, or 100%) sequence identity to any one of SEQ ID NOs: 53, 55, 57, 59, 61, 63, 65, 67, 69, 71, 73, 75, 77, 92 or 94, or to a fragment thereof that is at least about 300 (e.g., at least about 300, 400, 500, or 600) residues in length. In certain embodiments, isolated, synthetic, or recombinant nucleotides that are capable of hybridizing to any one of SEQ ID NOs: 53, 55, 57, 59, 61, 63, 65, 67, 69, 71, 73, 75, 77, 92 or 94, to a fragment of at least about 300 residues in length, or to a complement thereof, under low stringency, medium stringency, high stringency, or very high stringency conditions are provided.

In certain embodiments, the disclosure provides isolated, synthetic or recombinant polypeptides having at least about 60% (e.g., at least about 60%, 65%, 70%, 75%, 80%, 85%, 90%, 91%, 92%, 93%, 94%, 95%, 96%, 97%, 98%, 99%, or 100%) identity to any one of SEQ ID NOs:44, 54, 56, 58, 60, 62, 64, 66, 68, 70, 72, 74, 76, 78, 79, 93, and 95, over the full length catalytic domain (CD) or the carbohydrate binding module (CBM). The isolated, synthetic, or recombiant polypeptides can have β-glucosidase activity.

In some aspects, the disclosure provides isolated, synthetic or recombinant polypeptides having at least about 60% (e.g., at least about 60%, 65%, 70%, 75%, 80%, 85%, 90%, 91%, 92%, 93%, 94%, 95%, 96%, 97%, 98%, 99%, or 100%) sequence identity to any one of SEQ ID NOs: 52, 80-81, 206-207, over a region of at least about 10 (e.g., at least about 10, 15, 20, 25, 30, 35, 40, 45, 50, 55, 60, 65, 70, 75, 80, 85, 90, 95, 100, 125, 150, 175, 200, 225, 250, 275, 300) residues, or over the full length catalytic domain (CD) or the carbohydrate binding domain (CBM). In certain embodiments, the isolated, synthetic, or recombiant polypeptides have GH61/endoglucanase activity. By “GH61/endoglucanase activity” is meant that the polypeptide has glycosyl hydrolase family 61 enzyme activity and/or having endoglucanase activity. In some embodiments, the disclosure provides isolated, synthetic or recombinant polypeptides of at least about 50 (e.g., at least about 50, 100, 150, 200, 250, or 300) amino acid residues in length, comprising one or more of the sequence motifs selected from the group consisting of (1) SEQ ID NOs:84 and 88; (2) SEQ ID NOs:85 and 88; (3) SEQ ID NO:86; (4) SEQ ID NO:87; (5) SEQ ID NOs:84, 88 and 89; (6) SEQ ID NOs:85, 88, and 89; (7) SEQ ID NOs: 84, 88, and 90; (8) SEQ ID NOs: 85, 88 and 90; (9) SEQ ID NOs:84, 88 and 91; (10) SEQ ID NOs: 85, 88 and 91; (11) SEQ ID NOs: 84, 88, 89 and 91; (12) SEQ ID NOs: 84, 88, 90 and 91; (13) SEQ ID NOs: 85, 88, 89 and 91: and (14) SEQ ID NOs: 85, 88, 90 and 91. In certain embodiments, the polypeptide is a GH61 endoglucanase polypeptide (e.g., an EG IV polypeptide from a microorganism or another suitable source, including, without limitation, a T. reesei Eg4 enzyme). In some embodiments, the GH61 endoglucanase polypeptide is a variant, a mutant or a fusion polypeptide derived from T. reesei Eg4 (e.g., a polypeptide comprising at least about 60%, 65%, 70%, 75%, 80%, 85%, 90%, 91%, 92%, 93%, 94%, 95%, 96%, 97%, 98%, 99%, or 100% sequence identity to SEQ ID NO:52).

In some aspects, the disclosure provides an isolated, synthetic, or recombinant nucleotide encoding a polypeptide having at least about 60% (e.g., at least about 60%, 65%, 70%, 75%, 80%, 85%, 90%, 91%, 92%, 93%, 94%, 95%, 96%, 97%, 98%, 99%, or 100%) sequence identity to any one of SEQ ID NOs: 52, 80-81, and 206-207, over a region of at least about 10 (e.g., at least about 10, 15, 20, 25, 30, 35, 40, 45, 50, 55, 60, 65, 70, 75, 80, 85, 90, 95, 100, 125, 150, 175, 200, 225, 250, 275, 300) residues, or over the full length catalytic domain (CD) or the carbohydrate binding domain (CBM). For example, the isolated, synthetic, or recombiant nucleotide encodes a polypeptide having GH61/endoglucanase activity. In some embodiments, the disclosure provides an isolated, synthetic or recombinant nucleotide encoding a polypeptide of at least about 50 (e.g., at least about 50, 100, 150, 200, 250, or 300) amino acid residues in length, comprising one or more of the sequence motifs selected from the group consisting of (1) SEQ ID NOs:84 and 88; (2) SEQ ID NOs:85 and 88; (3) SEQ ID NO:86; (4) SEQ ID NO:87; (5) SEQ ID NOs:84, 88 and 89; (6) SEQ ID NOs:85, 88, and 89; (7) SEQ ID NOs: 84, 88, and 90; (8) SEQ ID NOs: 85, 88 and 90; (9) SEQ ID NOs:84, 88 and 91; (10) SEQ ID NOs: 85, 88 and 91; (11) SEQ ID NOs: 84, 88, 89 and 91; (12) SEQ ID NOs: 84, 88, 90 and 91; (13) SEQ ID NOs: 85, 88, 89 and 91: and (14) SEQ ID NOs: 85, 88, 90 and 91. For example, the nucleotide is one that encodes a polypeptide having at least about 60%, 65%, 70%, 75%, 80%, 85%, 90%, 91%, 92%, 93%, 94%, 95%, 96%, 97%, 98%, 99%, or 100% sequence identity to SEQ ID NO:52. In some embodiments, the nucleotide encodes a GH61 endoglucanase polypeptide (e.g., an EG IV polypeptide from a suitable organism, such as, without limitation, T. reesei Eg4).

In some aspects, the disclosure provides an isolated, synthetic, or recombinant polypeptide having at least about 70%, e.g., at least about 71%, 72%, 73%, 74%, 75%, 76%, 77%, 78%, 79%, 80%, 81%, 82%, 83%, 84%, 85%, 86%, 87%, 88%, 89%, 90%, 91%, 92%, 93%, 94%, 95%, 96%, 97%, 98%, or 99%, or complete (100%) sequence identity to a polypeptide of any one of SEQ ID NOs:2, 4, 6, 8, 10, 12, 14, 16, 18, 20, 22, 24, 26, 28, 30, 32, 34, 36, 38, 40, 42, 43, and 45, over a region of at least about 10, e.g., at least about 15, 20, 25, 30, 35, 40, 45, 50, 55, 60, 65, 70, 75, 80, 85, 90, 95, 100, 125, 150, 175, 200, 225, 250, 275, 300, 325, or 350 residues, or over the full length immature polypeptide, mature polypeptide, the catalytic domain (CD) or the carbohydrate binding domain (CBM).

In some aspects, the disclosure provides an isolated, synthetic, or recombinant nucleotide encoding a polypeptide having at least about 70%, (e.g., at least about 71%, 72%, 73%, 74%, 75%, 76%, 77%, 78%, 79%, 80%, 81%, 82%, 83%, 84%, 85%, 86%, 87%, 88%, 89%, 90%, 91%, 92%, 93%, 94%, 95%, 96%, 97%, 98%, or 99%, or complete (100%)) sequence identity to a polypeptide of any one of SEQ ID NOs:2, 4, 6, 8, 10, 12, 14, 16, 18, 20, 22, 24, 26, 28, 30, 32, 34, 36, 38, 40, 42, 43, and 45, over a region of at least about 10, e.g., at least about 15, 20, 25, 30, 35, 40, 45, 50, 55, 60, 65, 70, 75, 80, 85, 90, 95, 100, 125, 150, 175, 200, 225, 250, 275, 300, 325, or 350 residues, or over the full length immature polypeptide, the mature polypeptide, the catalytic domain (CD) or the carbohydrate binding domain (CBM). In some aspects, the disclosure provides an isolated, synthetic, or recombinant nucleotide having at least about 70% (e.g., at least about 71%, 72%, 73%, 74%, 75%, 76%, 77%, 78%, 79%, 80%, 81%, 82%, 83%, 84%, 85%, 86%, 87%, 88%, 89%, 90%, 91%, 92%, 93%, 94%, 95%, 96%, 97%, 98%, or 99%, or complete (100%)) sequence identity to any one of SEQ ID NOs: 1, 3, 5, 7, 9, 11, 13, 15, 17, 19, 21, 23, 25, 27, 29, 31, 33, 35, 37, 39, and 41, or to a fragment thereof. The fragment may be at least about 10, 20, 30, 40, 50, 60, 70, 80, 90, 100 residues in length. In some embodiments, the disclosure provides an isolated, synthetic, or recombinant nucleotide that hybridizes under low stringency conditions, medium stringency conditions, high stringency conditions, or very high stringency conditions to any one of SEQ ID NOs: 1, 3, 5, 7, 9, 11, 13, 15, 17, 19, 21, 23, 25, 27, 29, 31, 33, 35, 37, 39, and 41, or to a fragment or subsequence thereof.

Polypeptides sequences of the disclosure also include sequences encoded by the nucleic acids of the disclosure, e.g., those described in Section 5.1. below.

The disclosure also provides a chimeric or fusion protein comprising at least one domain of a polypeptide (e.g., the CD, the CBM, or both). The at least one domain can be operably linked to a second amino acid sequence, e.g., a signal peptide sequence. Thus the disclosure provides a first type of chimeric or fusion enzyme produced by expressing a nucleotide sequence comprising a signal sequence of a polypeptide of the disclosure operably linked to a second nucleotide sequence encoding a second, different polypeptide, e.g., a heterologous polypeptide that is not naturally associated with the signal sequence. The disclosure, e.g., provides a recombinant polypeptide comprising residues 1 to 13, 1 to 14, 1 to 15, 1 to 16, 1 to 17, 1 to 18, 1 to 19, 1 to 20, 1 to 21, 1 to 22, 1 to 23, 1 to 24, 1 to 25, 1 to 26, 1 to 27, 1 to 28, 1 to 28, 1 to 30, 1 to 31, 1 to 32, 1 to 33, 1 to 34, 1 to 35, 1 to 36, 1 to 37, 1 to 38, or 1 to 40 of, e.g., SEQ ID NO:2, 4, 6, 8, 10, 12, 14, 16, 18, 20, 22, 24, 26, 28, 30, 32, 34, 36, 38, 40, 42, 43, 45, 52, 54, 56, 58, 60, 62, 64, 66, 68, 70, 72, 74, 76, 78-83, 93, or 95, with a polypeptide that is not naturally associated thereto. Further chimeric or fusion polypeptides are described in Section 5.1.1. below.

The disclosure provides a second type of chimeric or fusion enzyme comprising a first contiguous stretch of amino acid residues of a first polypeptide sequence, which is operably linked to a second contiguous stretch of amino acid residues of a second polypeptide sequence. The first and/or the second contiguous stretches can optionally comprise signal peptides. Accordingly, this type of chimeric or fusion enzyme is obtained by expressing a polynucleotide comprising a first gene encoding the first contiguous stretch of amino acid residues of the first polypeptide sequence, and a second gene encoding the second contiguous stretch of amino acid residues of the second polypeptide sequence, wherein the first gene and second gene are directly and operably linked. In certain other embodiments, the chimeric or fusion strategy can be used to operably link 2 or more contiguous stretches of amino acid residues obtained from different enzymes, wherein the contiguous stretches are not naturally or natively linked or associated. In certain embodiments, the contiguous stretches of amino acid residues, which are operably linked, can be obtained from enzymes that have similar enzymatic activity but are heterologous to each other and/or to the host cell. In yet a further embodiment, the operably linked 2 or more contiguous stretches of amino acid residues can be further linked to a suitable signal peptide, as described herein. In yet another embodiment, the first contiguous stretch of amino acid residues and the second contiguous stretch of amino acid residues linked via a linker domain. In some embodiments, the first contiguous stretch of amino acid residues, the second contiguous stretch of amino acid residues, or the linker sequence can comprise the loop sequence, which is, e.g., about 3, 4, 5, 6, 7, 8, 9, 10, or 11 amino acid residues in length and and having an amino acid sequence of FDRRSPG (SEQ ID NO:204), or of FD(R/K)YNIT (SEQ ID NO:205). In certain embodiments, the loop sequence is derived from an enzyme different from the enzymes from which the first and the second contiguous stretches of amino acid residues are derived. In some embodiments, the resulting chimeric or fusion enzymes have improved stability, e.g., reflected in the stability against proteolysis or proteolytic degradation during storage under standard storage conditions, or during expression/production under standard expression or production conditions, as compared to each of the enzyme counterparts from which the chimeric parts are obtained.

For the present disclosure, chimeric or fusion enzymes are defined by the enzymatic activity of one of the originating enzyme from which the chimeric sequence is derived. For example, if one of the chimeric sequences is derived from or is a variant of a β-glucosidase, then, regardless of which enzyme(s) from which the other chimeric sequences of the same polypeptide are derived, the hybrid/chimera enzyme is referred to as a β-glucosidase polypeptide. For the purpose of the present disclosure, an “X polypeptide” encompasses a variant, a mutant, or a chimeric/fusion X polypeptide having X enzymatic activity.

The present disclosure therefore provides polypeptide and/or nucleotides or nucleic acids encoding polypeptides having hemicellulolytic activities or celluloytic activities.

Hemicellulolytic activities include, without limitation, xylanase, β-xylosidase, and/or L-α-arabinofuranosidase activities. Polypeptides having hemicellulolytic activity include, without limitation, a xylanase, a β-xylosidase, and/or an L-α-arabinofuranosidase. Polypeptides having cellulase activities include, without limitation, β-glucosidase activity or β-glucosidase enriched whole cellulase activity, and a GH61/endoglucanase activity or an endoglucanase enriched cellulase activity.

The disclosure additionally provides an expression cassette comprising a nucleic acid of the disclosure or a subsequence thereof. For example, the nucleic acid comprises at least about 60%, e.g., at least about 61%, 62%, 63%, 64%, 65%, 66%, 67%, 68%, 69%, 70%, 71%, 72%, 73%, 74%, 75%, 76%, 77%, 78%, 79%, 80%, 81%, 82%, 83%, 84%, 85%, 86%, 87%, 88%, 89%, 90%, 91%, 92%, 93%, 94%, 95%, 96%, 97%, 98%, or 99% sequence identity to a nucleic acid sequence of SEQ ID NO:53, 55, 57, 59, 61, 63, 65, 69, 71, 73, 75, 77, 92, 94, over a region of at least about 10 residues, e.g., at least about 10, 20, 30, 40, 50, 75, 90, 100, 150, 200, 250, 300, 350, 400, or 500 residues. In some aspects, the nucleic acid encodes a β-glucosidase polypeptide, which can, e.g., be a chimeric/fusion polypeptide derived from two or more β-glucosidase polypeptides and comprises two or more β-glucosidase sequences, wherein the first sequence is at least about 200 amino acid residues in length and comprises one or more or all of SEQ ID NOs:96-108, whereas the second sequence is at least about 50 amino acid residues in length, and comprises one or more or all of SEQ ID NOs:109-116, and optionally also a third sequence of about 3, 4, 5, 6 ,7 ,8 , 9, 10, or 11 amino acid residues in length and having an amino acid sequence of FDRRSPG (SEQ ID NO:204), or of FD(R/K)YNIT (SEQ ID NO:205), which is derived from a third β-glucosidase polypeptide different from the first or the second β-glucosidase polypeptide. In particular, the first of the two or more β-glucosidase sequences is one that is at least about 200 amino acid residues in length and comprises at least 2 (e.g., at least 2, 3, 4, or all) of the amino acid sequence motifs of SEQ ID NOs: 197-202, and the second of the two or more β-glucosidase is at least 50 amino acid residues in length and comprises SEQ ID NO:203, and optionally also a third sequence of about 3, 4, 5, 6 ,7 ,8 , 9, 10, or 11 amino acid residues in length and having an amino acid sequence of FDRRSPG (SEQ ID NO:204), or of FD(R/K)YNIT (SEQ ID NO:205), which is derived from a third β-glucosidase polypeptide different from the first or the second β-glucosidase polypeptide.

In some aspects, the disclosure provides an expression cassette comprising a nucleic acid encoding a polypeptide of at least about 60% (e.g., at least about 60%, 65%, 70%, 75%, 80%, 85%, 90%, 91%, 92%, 93%, 94%, 95%, 96%, 97%, 98%, or 99% sequence identity to any one of SEQ ID NOs: 52, 80-81, 206-207, or any one of the sequence motifs selected from the group consisting of: (1) SEQ ID NOs:84 and 88; (2) SEQ ID NOs:85 and 88; (3) SEQ ID NO:86; (4) SEQ ID NO:87; (5) SEQ ID NOs:84, 88 and 89; (6) SEQ ID NOs:85, 88, and 89; (7) SEQ ID NOs: 84, 88, and 90; (8) SEQ ID NOs: 85, 88 and 90; (9) SEQ ID NOs:84, 88 and 91; (10) SEQ ID NOs: 85, 88 and 91; (11) SEQ ID NOs: 84, 88, 89 and 91; (12) SEQ ID NOs: 84, 88, 90 and 91; (13) SEQ ID NOs: 85, 88, 89 and 91: and (14) SEQ ID NOs: 85, 88, 90 and 91.

In some aspects, the disclosure provides an expression cassette comprising a nucleic acid encoding a polypeptide of at least about 70% (e.g., at least about 70%, 75%, 80%, 85%, 90%, 91%, 92%, 93%, 94%, 95%, 96%, 97%, 98%, or 99%) sequence identity to any one of SEQ ID NOs:2, 4, 6, 8, 10, 12, 14, 16, 18, 20, 22, 24, 26, 28, 30, 32, 34, 36, 38, 40, 42, 43, and 45, over a region of at least about 10 residues, e.g., at least about 10, 20, 30, 40, 50, 75, 90, 100, 150, 200, 250, 300, 350, 400, or 500 residues. In some aspects, the disclosure provides an expression cassette comprising a nucleic acid that hybridizes under low stringency conditions, medium stringency conditions, or high stringency conditions to any one of SEQ ID NOs: 1, 3, 5, 7, 9, 11, 13, 15, 17, 19, 21, 23, 25, 27, 29, 31, 33, 35, 37, 39, and 41, or to a fragment or subsequence thereof, wherein the fragment or subsequence is at least about, e.g., 10, 20, 30, 40, 50, 75, 100, 125, 150, 200, 250 residues in length.

In some aspects, the nucleic acid of the expression cassette is optionally operably linked to a promoter. The promoter can be, e.g., a fungal, viral, bacterial, mammalian, or plant promoter. The promoter can be a constitutive promoter or an inducible promoter, expressable in, e.g., filamentous fungi. A suitable promoter can be derived from a filamentous fungus. For example, the promoter can be a cellobiohydrolase 1 (“cbh1”) gene promoter from T. reesei.

In some aspects, the disclosure provides a recombinant cell engineered to express a nucleic acid or an expression cassette of the disclosure. The recombinant cell is desirably a bacterial cell, a mammalian cell, a fungal cell, a yeast cell, an insect cell or a plant cell. For example, the recombinant cell is a recombinant filamentous fungal cell, such as a Trichoderma, Humicola, Fusarium, Aspergillus, Neurospora, Penicillium, Cephalosporium, Achlya, Podospora, Endothia, Mucor, Cochliobolus, Pyricularia, or Chrysosporium cell.

The disclosure also provides methods of producing a recombinant polypeptide comprising: (a) culturing a host cell engineered to express a polypeptide of the disclosure; and (b) recovering the polypeptide. The recovery of the polypeptide includes, e.g., recovery of the fermentation broth comprising the polypeptide. The fermentation broth may be used with minimum post-production processing, e.g., purification, ultrafiltration, a cell kill step, etc., and in that case it is said that the fermentation broth is used in a whole broth formulation. Alternatively, the polypeptide can be recovered using further purification step(s).

In a further aspect, the invention pertains to certain engineered enzyme compositions comprising 2 or more, 3 or more, 4 or more, or 5 or more, polypeptides (including suitable variants, mutants, or fusion/chimeric polypeptides) of the invention, wherein the enzyme compositions can hydrolyze one or more components of a lignocellulosic biomass material. Such components include, e.g., hemicellulose and, optionally, cellulose. Suitable lignocellulosic biomass materials include, without limitation, seeds, grains, tubers, plant waste or byproducts of food processing or industrial processing (e.g., stalks), corn (including, e.g., cobs, stover, and the like), grasses (e.g., Indian grass, such as Sorghastrum nutans; or, switchgrass, e.g., Panicum species, such as Panicum virgatum), perennial canes, e.g., giant reeds, wood (including, e.g., wood chips, processing waste), paper, pulp, recycled paper (e.g., newspaper). The enzyme blends/compositions can be used to hydrolyze cellulose comprising a linear chain of 3-1,4-linked glucose moieties, or hemicellulose, of a complex structure that varies from plant to plant.

The engineered enzyme compositions of the invention can comprise a number of different polypeptides having, e.g., hemicellulase activity or cellulase activity. The hemicellulase activity can be a xylanase activity, an arabinofuranosidase activity, or a xylosidase activity. The cellulase activity can be a glocosidase activity, a cellobiohydrolase activity, or an endoglucanase activity. A polypeptide of the enzyme composition of the invention can be one that has one or more of the hemicellulase activities and/or cellulase activities. For example, a polypeptide of the enzyme composition can have both a β-xylosidase activity and an L-α-arabinofuranosidase activity. Also, two or more polypeptides of a given enzyme composition can have the same or similar enzymatic activities. For example, more than one polypeptide in the composition can independently have endoglucanase, β-xylosidase, or β-glucosidase activity.

Suitable polypeptides of the invention can be isolated from naturally-occurring sources. For example, one or more polypeptides can be purified or substantially purified from naturally-occurring sources. In another example, one or more polypeptides can be recombinantly produced by an engineered organism, such as by a recombinant bacterium or fungus. One or more polypeptides may be overexpressed by a recombinant organism. One or more polypeptides can be expressed or co-expressed with one or more heterologous (i.e., not naturally occurring in the same organisms) polypeptides. Genes encoding one or more polypeptides of the invention may be integrated into the genetic materials of a recombinant host organism, e.g., a host fungal cell or a host bacterial cell, which can then be used to produce the gene products.

The enzyme compositions of the invention can be naturally occurring or engineered compositions. The term “naturally occurring enzyme composition” refers to a composition that exists in nature, e.g., one that is directly derived from an unmodified organism grown under conditions of its native environment. The term “engineered composition” refers to a composition wherein at least one enzyme is (1) recombinantly produced; (2) produced by an organism via expression of a heterologous gene; and/or (3) is present in an amount or relative weight percent that is more or less than what is present in a naturally-occurring enzyme composition comprising identical or similar types of enzymes. A “recombinantly produced” enzyme is one produced via recombinant means. A recombinantly produced enzyme can be present in a mixture wherein the recombinantly produced enzyme is among mixtures of other enzymes that are not naturally co-existing. Moreover an engineered composition can also be one produced by an organism found in nature (i.e., an organism that is unmodified) grown under conditions different from those found in its native habitat.

The polypeptides, mixture thereof, and/or the engineered enzyme compositions of the invention can be used to hydrolyze biomass materials or other suitable feedstocks. The enzyme compositions desirably comprise mixtures of 2 or more, 3 or more, 4 or more, or even 5 or more polypeptides of the invention, selected from xylanases, xylosidases, cellobiohydrolases, endoglucanases, glucosidases, and optionally arabinofuranosidases, and/or other enzymes that can catalyze or aid the digestion or conversion of hemicellulose materials to fermentable sugars. Suitable glucosidases include, e.g., a number of β-glucosidases, including, without limitation, those having at least about 60% (e.g., at least about 60%, 65%, 70%, 75%, 80%, 85%, 90%, 91%, 92%, 93%, 94%, 95%, 96%, 97%, 98%, or 99%) identity to any one of SEQ ID NOs: 54, 56, 58, 60, 62, 64, 66, 68, 70, 72, 74, 76, 78, 79, 93, and 95, over a region of at least about 10 (e.g., at least about 10, 15, 20, 25, 30, 35, 40, 45, 50, 55, 60, 65, 70, 75, 80, 85, 90, 95, 100, 125, 150, 175, 200, 225, 250, 275, 300) residues. Suitable glucosidases also include, e.g., a chimeric/fusion β-glucosidase polypeptide comprising two or more β-glucosidase sequences, wherein the first sequence derived from a first β-glucosidase is at least about 200 amino acid residues in length and comprises one or more or all of the amino acid sequence motifs of SEQ ID NOs: 96-108, whereas the second sequence derived from a second β-glucosidase is at least about 50 amino acid residues in length and comprises one or more or all of the amino acid sequence motifs of SEQ ID NOs:109-116, and optionally also a third sequence of 3, 4, 5, 6, 7, 8, 9, 10, or 11 amino acid residues in length encoding a loop sequence derived from a third β-glucosidase, having an amino acid sequence of FDRRSPG (SEQ ID NO:204), or of FD(R/K)YNIT (SEQ ID NO:205). In particular, the first of the two or more β-glucosidase sequences is one that is at least about 200 amino acid residues in length and comprises at least 2 (e.g., at least 2, 3, 4, or all) of the amino acid sequence motifs of SEQ ID NOs: 197-202, and the second of the two or more β-glucosidase is at least 50 amino acid residues in length and comprises SEQ ID NO:203, and optionally also a third sequence of about 3, 4, 5, 6 ,7 ,8 , 9, 10, or 11 amino acid residues in length and having an amino acid sequence of FDRRSPG (SEQ ID NO:204), or of FD(R/K)YNIT (SEQ ID NO:205), which is derived from a third β-glucosidase polypeptide different from the first or the second β-glucosidase polypeptide.

Suitable endoglucanses include, e.g., one or more GH61 endoglucanases including, without limitation, those having at least about 60% (e.g., at least about 60%, 65%, 70%, 75%, 80%, 85%, 90%, 91%, 92%, 93%, 94%, 95%, 96%, 97%, 98%, or 99%) sequence identity to any one of SEQ ID NOs: 52, 80-81, 206-207, over a region of at least about 10 (e.g., at least about 10, 15, 20, 25, 30, 35, 40, 45, 50, 55, 60, 65, 70, 75, 80, 85, 90, 95, 100, 125, 150, 175, 200, 225, 250, 275, 300) residues. Suitable endoglucanases can also include polypeptides comprising one or more sequence motifs selected from the group consisting of: (1) SEQ ID NOs:84 and 88; (2) SEQ ID NOs:85 and 88; (3) SEQ ID NO:86; (4) SEQ ID NO:87; (5) SEQ ID NOs:84, 88 and 89; (6) SEQ ID NOs:85, 88, and 89; (7) SEQ ID NOs: 84, 88, and 90; (8) SEQ ID NOs: 85, 88 and 90; (9) SEQ ID NOs:84, 88 and 91; (10) SEQ ID NOs: 85, 88 and 91; (11) SEQ ID NOs: 84, 88, 89 and 91; (12) SEQ ID NOs: 84, 88, 90 and 91; (13) SEQ ID NOs: 85, 88, 89 and 91: and (14) SEQ ID NOs: 85, 88, 90 and 91.

The other enzymes that can digest hemicellulose to fermentable sugars include, without limitation, a cellulase, a hemicellulase, or a composition comprising a cellulase or a hemicellulase. Suitable other polypeptides that can also be present, including, e.g., cellobiose dehydrogenases. An engineered enzyme composition of the invention can comprise mixtures of 2 or more, 3 or more, 4 or more, or even 5 or more polypeptides of the invention, selected from xylanases, xylosidases, arabinofuranosidases, and a panel of cellulases. The engineered enzyme composition can optionally also comprise one or more cellobiose dehydrogenases. The whole cellulase composition can be one enriched with a β-glucosidase polypeptide, or one enriched with an endoglucanase polypeptide, or one enriched with both a β-glucosidase polypeptide and an endoglucanase polypeptide. In some embodiments, the endoglucanse polypeptide can be one that is a member of GH61 family, e.g., one having at least about 60% (e.g., at least about 60%, 65%, 70%, 75%, 80%, 85%, 90%, 91%, 92%, 93%, 94%, 95%, 96%, 97%, 98%, or 99%) sequence identity to any one of SEQ ID NOs: 52, 80-81, 206-207, over a region of at least about 10 (e.g., at least about 10, 15, 20, 25, 30, 35, 40, 45, 50, 55, 60, 65, 70, 75, 80, 85, 90, 95, 100, 125, 150, 175, 200, 225, 250, 275, 300) residues. The endoglucanase polypeptide can be one that comprises one or more sequence motifs selected from the group consisting of: (1) SEQ ID NOs:84 and 88; (2) SEQ ID NOs:85 and 88; (3) SEQ ID NO:86; (4) SEQ ID NO:87; (5) SEQ ID NOs:84, 88 and 89; (6) SEQ ID NOs:85, 88, and 89; (7) SEQ ID NOs: 84, 88, and 90; (8) SEQ ID NOs: 85, 88 and 90; (9) SEQ ID NOs:84, 88 and 91; (10) SEQ ID NOs: 85, 88 and 91; (11) SEQ ID NOs: 84, 88, 89 and 91; (12) SEQ ID NOs: 84, 88, 90 and 91; (13) SEQ ID NOs: 85, 88, 89 and 91: and (14) SEQ ID NOs: 85, 88, 90 and 91. For example, the endoglucanase polypeptide can be an EGIV from a suitable organism, such as T. reesei Eg4. In some embodiments, the β-glucosidase polypeptide can be one that has at least about having at least about 60% (e.g., at least about 60%, 65%, 70%, 75%, 80%, 85%, 90%, 91%, 92%, 93%, 94%, 95%, 96%, 97%, 98%, or 99%) identity to any one of SEQ ID NOs: 54, 56, 58, 60, 62, 64, 66, 68, 70, 72, 74, 76, 78, 79, 93, and 95, over a region of at least about 10 (e.g., at least about 10, 15, 20, 25, 30, 35, 40, 45, 50, 55, 60, 65, 70, 75, 80, 85, 90, 95, 100, 125, 150, 175, 200, 225, 250, 275, or 300) residues.

A first non-limiting example of an engineered enzyme composition of the invention comprises 4 polypeptides: (1) a first polypeptide having xylanase activity, (2) a second polypeptide having xylosidase activity, (3) a third polypeptide having arabinofuranosidase activity, and (4) a fourth polypeptide having β-glucosidase activity. In certain embodiments, the fourth polypeptide having β-glucosidase activity has at least about 60% (e.g., at least about 60%, 65%, 70%, 75%, 80%, 85%, 90%, 91%, 92%, 93%, 94%, 95%, 96%, 97%, 98%, or 99%) sequence identity to any one of SEQ ID NOs: 54, 56, 58, 60, 62, 64, 66, 68, 70, 72, 74, 76, 78, 79, 93, and 95, over a region of at least about 10 (e.g., at least about 10, 15, 20, 25, 30, 35, 40, 45, 50, 55, 60, 65, 70, 75, 80, 85, 90, 95, 100, 125, 150, 175, 200, 225, 250, 275, 300) residues. In certain embodiments, the fourth polypeptide having β-glucosidase is a chimeric/fusion polypeptide comprising two or more β-glucosidase sequences, wherein the first sequence derived from a first β-glucosidase is at least about 200 amino acid residues in length and comprises one or more or all of the sequence motifs of SEQ ID NOs: 96-108, whereas the second sequence derived from a second β-glucosidase is at least about 50 amino acid residues in length and comprises one or more or all of the sequence motifs of SEQ ID NOs:109-116, and optionally, also a third sequence of 3, 4, 5, 6, 7, 8, 9, 10, or 11 amino acid residues in length encoding a loop sequence derived from a third β-glucosidase having an amino acid sequence of FDRRSPG (SEQ ID NO:204), or of FD(R/K)YNIT (SEQ ID NO:205). In particular, the first of the two or more β-glucosidase sequences is one that is at least about 200 amino acid residues in length and comprises at least 2 (e.g., at least 2, 3, 4, or all) of the amino acid sequence motifs of SEQ ID NOs: 197-202, and the second of the two or more β-glucosidase is at least 50 amino acid residues in length and comprises SEQ ID NO:203, and optionally also a third sequence of about 3, 4, 5, 6 ,7 ,8 , 9, 10, or 11 amino acid residues in length and having an amino acid sequence of FDRRSPG (SEQ ID NO:204), or of FD(R/K)YNIT (SEQ ID NO:205), which is derived from a third β-glucosidase polypeptide different from the first or the second β-glucosidase polypeptide. For example, the fourth polypeptide having β-glucosidase activity comprises a first sequence having least about 60% sequence identity to an at least 200-residue stretch of Fv3C (SEQ ID NO:60), e.g., an at least 200-residue stretch from the N-terminus, or an amino acid position near to the N-terminus, of SEQ ID NO:60, and a second sequence having at least about 60% sequence identity to an at least 50-residue stretch of T. reesei Bgl3 (Tr3B, SEQ ID NO:64), e.g., an at least 50-residue stretch from the C-terminus, or an amino acid position near to the C-terminus of SEQ ID NO:64. The fourth polypeptide can further comprise a third sequence of about 3, 4, 5, 6, 7, 8, 9, 10, or 11 amino acid residues that is derived from a sequence of equal length from Te3A (SEQ ID NO:66), or comprises an amino acid sequence of FDRRSPG (SEQ ID NO:204), or of FD(R/K)YNIT (SEQ ID NO:205). In some embodiments, the fourth polypeptide comprises a sequence that has at least about 60% sequence identity to SEQ ID NO:93 or 95, or to a subsequence or fragment of at least about 20, 30, 40, 50, 60, 70, or more residues of SEQ ID NO: 93 or 95.

In some embodiments, the engineered enzyme composition further comprises a fifth polypeptide having GH61/endoglucanase activity or alternatively, a GH61 endoglucanase-enriched whole cellulase. For example, the polypeptide having GH61/endoglucanase activity is an EGIV polypeptide, e.g., a T. reesei Eg4. The GH61 endoglucanase-enriched whole cellulase is a whole cellulase enriched with an EGIV polypeptide, e.g., a T. reesei Eg4. In some embodiments, the fifth polypeptide has at least about 60% (e.g., at least about 60%, 65%, 70%, 75%, 80%, 85%, 90%, 91%, 92%, 93%, 94%, 95%, 96%, 97%, 98%, or 99%) sequence identity to any one of SEQ ID NOs: 52, 80-81, 206-207 over a region of at least about 10 (e.g., at least about 10, 15, 20, 25, 30, 35, 40, 45, 50, 55, 60, 65, 70, 75, 80, 85, 90, 95, 100, 125, 150, 175, 200, 225, 250, 275, 300) residues, or comprises one or more sequence motifs selected from the group consisting of: (1) SEQ ID NOs:84 and 88; (2) SEQ ID NOs:85 and 88; (3) SEQ ID NO:86; (4) SEQ ID NO:87; (5) SEQ ID NOs:84, 88 and 89; (6) SEQ ID NOs:85, 88, and 89; (7) SEQ ID NOs: 84, 88, and 90; (8) SEQ ID NOs: 85, 88 and 90; (9) SEQ ID NOs:84, 88 and 91; (10) SEQ ID NOs: 85, 88 and 91; (11) SEQ ID NOs: 84, 88, 89 and 91; (12) SEQ ID NOs: 84, 88, 90 and 91; (13) SEQ ID NOs: 85, 88, 89 and 91; and (14) SEQ ID NOs: 85, 88, 90 and 91. In some embodiments, the enzyme composition further comprises a cellobiose dehydrogenase.

In some embodiments, the first polypeptide having xylanase activity has at least about 70% sequence identity to any one of SEQ ID NOs: 24, 26, 42, and 43, or to a mature sequence thereof. For example, the first polypeptide is AfuXyn2, AfuXyn5, T. reesei Xyn3, or T. reesei Xyn2.

In some embodiments, the second polypeptide having xylosidase activity is selected from a Group 1 or Group 2 β-xylosidase polypeptides. Group 1 β-xylosidase polypeptides have at least about 70% sequence identity to any one of SEQ ID NOs: 2 and 10, or to a mature sequences thereof. For example, Group 1 β-xylosidase can be Fv3A or Fv43A. Group 2 β-xylosidase polypeptides have at least about 70% sequence identity to any one of SEQ ID NOs:4, 6, 8, 10, 12, 14, 16, 18, 28, 30, and 45, or to a mature sequence thereof. For example, Group 2 β-xylosidases can be Pf43A, Fv43E, Fv39A, Fv43B, Pa51A, Gz43A, Fo43A, Fv43D, Pf43B, or T. reesei Bxl1.

In some embodiments, the third polypeptide having arabinofuranosidase activity has at least about 70% sequence identity to any one of SEQ ID NOs:12, 14, 20, 22, and 32, or to a mature sequence thereof. For example, the third polypeptide can be Fv43B, Pa51A, Af43A, Pf51A, or Fv51A.

The first, second, third, fourth, or fifth polypeptide can be isolated or purified form a naturally-occurring source. Alternatively, it can be expressed or overexpressed by a recombinant host cell. It can be added to an enzyme composition in an isolated or purified form. It can be expressed or overexpressed by a host organism or host cell as a part of culture mixture, e.g., a fermentation broth. In some embodiments, a gene encoding such polypeptide can be integrated into the genetic material of the host organism, which allows the expression of the encoded polypeptides by that organism.

A second non-limiting example of an engineered enzyme composition of the invention comprises: (1) a first polypeptide having xylanase activity, (2) a second polypeptide having xylosidase activity, (3) a third polypeptide having arabinofuranosidase activity, and (4) a β-glucosidase-enriched whole cellulase composition. In certain embodiments, the β-glucosidase-enriched whole cellulase composition is enriched with a β-glucosidase polypeptide having at least about 60% (e.g., at least about 60%, 65%, 70%, 75%, 80%, 85%, 90%, 91%, 92%, 93%, 94%, 95%, 96%, 97%, 98%, or 99%) sequence identity to any one of SEQ ID NOs: 54, 56, 58, 60, 62, 64, 66, 68, 70, 72, 74, 76, 78, 79, 93, and 95, over a region of at least about 10 (e.g., at least about 10, 15, 20, 25, 30, 35, 40, 45, 50, 55, 60, 65, 70, 75, 80, 85, 90, 95, 100, 125, 150, 175, 200, 225, 250, 275, 300) residues. In certain embodiments, the β-glucosidase-enriched whole cellulase composition is enriched with a chimeric/fusion β-glucosidase polypeptide comprising 2 or more β-glucosidase sequences, wherein the first sequence derived from a first β-glucosidase is at least about 200 amino acid residues in length and comprises one or more or all of the sequence motifs of SEQ ID NOs: 96-108, whereas the second sequence derived from a second β-glucosidase is at least about 50 amino acid residues in length and comprises one or more or all of the sequence motifs of SEQ ID NOs:109-116, and optionally also a third sequence of 3, 4, 5, 6, 7, 8, 9, 10, or 11 amino acid residues in length encoding a loop sequence derived from a third β-glucosidase, having an amino acid sequence of FDRRSPG (SEQ ID NO:204), or of FD(R/K)YNIT (SEQ ID NO:205). In particular, the first of the two or more β-glucosidase sequences is one that is at least about 200 amino acid residues in length and comprises at least 2 (e.g., at least 2, 3, 4, or all) of the amino acid sequence motifs of SEQ ID NOs: 197-202, and the second of the two or more β-glucosidase is at least 50 amino acid residues in length and comprises SEQ ID NO:203, and optionally also a third sequence of about 3, 4, 5, 6, 7, 8, 9, 10, or 11 amino acid residues in length and having an amino acid sequence of FDRRSPG (SEQ ID NO:204), or of FD(R/K)YNIT (SEQ ID NO:205), which is derived from a third β-glucosidase polypeptide different from the first or the second β-glucosidase polypeptide. For example, the β-glucosidase-enriched whole cellulase composition is enriched with a β-glucosidase polypeptide comprising a first sequence having least about 60% sequence identity to an at least 200-residue stretch of Fv3C (SEQ ID NO:60), e.g., an at least 200-residue stretch from the N-terminus, or from a residue that is near to the N-terminus of SEQ ID NO:60, and a second sequence having at least about 60% sequence identity to an at least 50-residue stretch of T. reesei Bgl3 (Tr3B, SEQ ID NO:64), e.g., an at least 50-residue stretch from the C-terminus or from a residue near to the C-terminus of SEQ ID NO:64. The β-glucosidase-enriched whole cellulase composition is enriched with a β-glucosidase polypeptide further comprising a third sequence of about 3, 4, 5, 6, 7, 8, 9, 10, or 11 amino acid residues that is derived from a sequence of equal length from Te3A (SEQ ID NO:66), or have an amino acid sequence of FDRRSPG (SEQ ID NO:204), or of FD(R/K)YNIT (SEQ ID NO:205). In some embodiments, the fourth polypeptide comprises a sequence that has at least about 60% sequence identity to SEQ ID NO:93 or 95, or to a subsequence or fragment of at least about 20, 30, 40, 50, 60, 70, or more residues of SEQ ID NO: 93 or 95.

In some embodiments, the engineered enzyme composition further comprises a fourth polypeptide having GH61/endoglucanase activity, or alternatively, a GH61 endoglucanase-enriched whole cellulase. For example, the polypeptide having GH61/endoglucanase activity is an EGIV polypeptide, e.g., a T. reesei Eg4 polypeptide. In some embodiments, the GH61 endoglucanase-enriched whole cellulase is a whole cellulase enriched with an EGIV polypeptide, e.g., a T. reesei Eg4 polypeptide.

In some embodiments, the fourth polypeptide is one having at least about 60% (e.g., at least about 60%, 65%, 70%, 75%, 80%, 85%, 90%, 91%, 92%, 93%, 94%, 95%, 96%, 97%, 98%, or 99%) identity to any one of SEQ ID NOs: 52, 80-81, 206-207, over a region of at least about 10 (e.g., at least about 10, 15, 20, 25, 30, 35, 40, 45, 50, 55, 60, 65, 70, 75, 80, 85, 90, 95, 100, 125, 150, 175, 200, 225, 250, 275, 300) residues, or comprises one or more sequence motifs selected from the group consisting of: (1) SEQ ID NOs:84 and 88; (2) SEQ ID NOs:85 and 88; (3) SEQ ID NO:86; (4) SEQ ID NO:87; (5) SEQ ID NOs:84, 88 and 89; (6) SEQ ID NOs:85, 88, and 89; (7) SEQ ID NOs: 84, 88, and 90; (8) SEQ ID NOs: 85, 88 and 90; (9) SEQ ID NOs:84, 88 and 91; (10) SEQ ID NOs: 85, 88 and 91; (11) SEQ ID NOs: 84, 88, 89 and 91; (12) SEQ ID NOs: 84, 88, 90 and 91; (13) SEQ ID NOs: 85, 88, 89 and 91: and (14) SEQ ID NOs: 85, 88, 90 and 91. In some embodiments, the enzyme composition further comprises a cellobiose dehydrogenase.

In some embodiments, the first polypeptide having xylanase activity has at least about 70% sequence identity to any one of SEQ ID NOs: 24, 26, 42, and 43, or to a mature sequence thereof. For example, the first polypeptide is AfuXyn2, AfuXyn5, T. reesei Xyn3, or T. reesei Xyn2.

In some embodiments, the second polypeptide having xylosidase activity is selected from either a Group 1 or Group 2 β-xylosidase polypeptide. Group 1 β-xylosidase polypeptides have at least about 70% sequence identity to any one of SEQ ID NOs: 2 and 10, or to mature sequences thereof. For example, Group 1 β-xylosidase is Fv3A or Fv43A. Group 2 β-xylosidase polypeptides have at least about 70% sequence identity to any one of SEQ ID NOs:4, 6, 8, 10, 12, 14, 16, 18, 28, 30, and 45, or to a mature sequence thereof. For example, Group 2 β-xylosidases can be Pf43A, Fv43E, Fv39A, Fv43B, Pa51A, Gz43A, Fo43A, Fv43D, Pf43B, or T. reesei Bxl1.

In some embodiments, the third polypeptide having arabinofuranosidase activity has at least about 70% sequence identity to any one of SEQ ID NOs:12, 14, 20, 22, and 32, or to a mature sequence thereof. For example, the third polypeptide can be Fv43B, Pa51A, Af43A, Pf51A, or Fv51A.

The first, second, third, or fourth polypeptide can be isolated or purified form a naturally-occurring source. Alternatively, it can be expressed or overexpressed by a recombinant host cell. It can be added to an enzyme composition in an isolated or purified form. It can be expressed or overexpressed by a host organism or host cell as a part of culture mixture, e.g., a fermentation broth. In some embodiments, a gene encoding such polypeptide can be integrated into the genetic material of the host organism, which allows the expression of the encoded polypeptides by that organism.

A third non-limiting example of an engineered enzyme composition of the invention comprises (1) a first polypeptide having xylanase activity; (2) a second polypeptide having xylosidase activity; (3) a third polypeptide having arabinofuranosidase activity; and (4) a fourth polypeptide having a GH61/endoglucanase activity, or a GH61 endoglucanase-enriched whole cellulase. In some embodiments, the fourth polypeptide having GH61/endoglucanase activity is an EGIV polypeptide. In some embodiments, the polypeptide having GH61/endoglucanase activity is an EGIV polypeptide from a suitable microorganism, e.g., a T. reesei Eg4 polypeptide. In some embodiments, the GH61 endoglucanase-enriched whole cellulase is a whole cellulase enriched with an EGIV polypeptide, e.g., a T. reesei Eg4 polypeptide. In some embodiments, the fourth polypeptide is one having at least about 60% (e.g., at least about 60%, 65%, 70%, 75%, 80%, 85%, 90%, 91%, 92%, 93%, 94%, 95%, 96%, 97%, 98%, or 99%) sequence identity to any one of SEQ ID NOs: 52, 80-81, 206-207, over a region of at least about 10 (e.g., at least about 10, 15, 20, 25, 30, 35, 40, 45, 50, 55, 60, 65, 70, 75, 80, 85, 90, 95, 100, 125, 150, 175, 200, 225, 250, 275, 300) residues, or one that comprises one or more sequence motifs selected from the group consisting of: (1) SEQ ID NOs:84 and 88; (2) SEQ ID NOs:85 and 88; (3) SEQ ID NO:86; (4) SEQ ID NO:87; (5) SEQ ID NOs:84, 88 and 89; (6) SEQ ID NOs:85, 88, and 89; (7) SEQ ID NOs: 84, 88, and 90; (8) SEQ ID NOs: 85, 88 and 90; (9) SEQ ID NOs:84, 88 and 91; (10) SEQ ID NOs: 85, 88 and 91; (11) SEQ ID NOs: 84, 88, 89 and 91; (12) SEQ ID NOs: 84, 88, 90 and 91; (13) SEQ ID NOs: 85, 88, 89 and 91: and (14) SEQ ID NOs: 85, 88, 90 and 91. The composition can further comprise a cellobiose dehydrogenase.

In some embodiments, the first polypeptide having xylanase activity has at least about 70% sequence identity to any one of SEQ ID NOs: 24, 26, 42, and 43, or to a mature sequence thereof. For example, the first polypeptide can be AfuXyn2, AfuXyn5, T. reesei Xyn3, or T. reesei Xyn2.

In some embodiments, the second polypeptide having xylosidase activity can be one selected from either a Group 1 or Group 2 β-xylosidase polypeptides. Group 1 β-xylosidase polypeptides have at least about 70% sequence identity to any one of SEQ ID NOs: 2 and 10, or to a mature sequence thereof. For example, Group 1 β-xylosidase can be Fv3A or Fv43A. Group 2 β-xylosidase polypeptides have at least about 70% sequence identity to any one of SEQ ID NOs:4, 6, 8, 10, 12, 14, 16, 18, 28, 30, and 45, or to a mature sequence thereof. For example, Group 2 β-xylosidases can be Pf43A, Fv43E, Fv39A, Fv43B, Pa51A, Gz43A, Fo43A, Fv43D, Pf43B, or T. reesei Bxl1.

In some embodiments, the third polypeptide having arabinofuranosidase activity has at least about 70% sequence identity to any one of SEQ ID NOs:12, 14, 20, 22, and 32, or to a mature sequence thereof. For example, the third polypeptide can be Fv43B, Pa51A, Af43A, Pf51A, or Fv51A.

The first, second, third, or fourth, or other polypeptide can be isolated or purified form a naturally-occurring source. Alternatively, it can be expressed or overexpressed by a recombinant host cell. It can be added to an enzyme composition in an isolated or purified form. It can be expressed or overexpressed by a host organism or host cell as a part of culture mixture, e.g., a fermentation broth. In some embodiments, a gene encoding such a polypeptide can be integrated into the genetic material of the host organism, which allows the expression of the encoded polypeptides by that organism.

A fourth non-limiting example of an engineered enzyme composition of the invention comprises (1) a first polypeptide having xylosidase activity, (2) a second polypeptide (which differs from the first polypeptide) having xylosidase activity, (3) a third polypeptide having arabinofuranosidase activity, and (4) a fourth polypeptide having β-glucosidase activity. In certain embodiments, the fourth polypeptide has at least about 60% (e.g., at least about 60%, 65%, 70%, 75%, 80%, 85%, 90%, 91%, 92%, 93%, 94%, 95%, 96%, 97%, 98%, or 99%) identity to any one of SEQ ID NOs: 54, 56, 58, 60, 62, 64, 66, 68, 70, 72, 74, 76, 78, 79, 93, and 95, over a region of at least about 10 (e.g., at least about 10, 15, 20, 25, 30, 35, 40, 45, 50, 55, 60, 65, 70, 75, 80, 85, 90, 95, 100, 125, 150, 175, 200, 225, 250, 275, 300) residues. In certain embodiments, the fourth polypeptide is a chimeric/fusion β-glucosidase polypeptide comprising two or more β-glucosidase sequences, wherein the first sequence derived from a first β-glucosidase is at least about 200 amino acid residues in length and comprises one or more or all of the sequence motifs of SEQ ID NOs: 96-108, whereas the second sequence derived from a second β-glucosidase is at least about 50 amino acid residues in length and comprises one or more or all of the sequence motifs of SEQ ID NOs:109-116, and optionally also a third sequence of 3, 4, 5, 6, 7, 8, 9, 10, or 11 amino acid residues in length encoding a loop sequence derived from a third β-glucosidase, having an amino acid sequence of FDRRSPG (SEQ ID NO:204), or of FD(R/K)YNIT (SEQ ID NO:205).

In particular, the first of the two or more β-glucosidase sequences is one that is at least about 200 amino acid residues in length and comprises at least 2 (e.g., at least 2, 3, 4, or all) of the amino acid sequence motifs of SEQ ID NOs: 197-202, and the second of the two or more β-glucosidase is at least 50 amino acid residues in length and comprises SEQ ID NO:203, and optionally also a third sequence of about 3, 4, 5, 6, 7, 8, 9, 10, or 11 amino acid residues in length and having an amino acid sequence of FDRRSPG (SEQ ID NO:204), or of FD(R/K)YNIT (SEQ ID NO:205), which is derived from a third β-glucosidase polypeptide different from the first or the second β-glucosidase polypeptide. For example, the fourth polypeptide comprises a first sequence having least about 60% sequence identity to an at least 200-residue stretch of Fv3C (SEQ ID NO:60), e.g., an at least 200-residue stretch from the N-terminus or from a residue near to the N-terminus of SEQ ID NO:60, and a second sequence having at least about 60% sequence identity to an at least 50-residue stretch of T. reesei Bgl3 (Tr3B, SEQ ID NO:64), e.g., an at least 50-residue stretch from the C-terminus or from a residue close to the C-terminus of SEQ ID NO:64. The fourth polypeptide further comprises a third sequence of about 3, 4, 5, 6, 7, 8, 9, 10, or 11 amino acid residues that is derived from a sequence of equal length from Te3A (SEQ ID NO:66), or has an amino acid sequence of FDRRSPG (SEQ ID NO:204), or of FD(R/K)YNIT (SEQ ID NO:205). In some embodiments, the fourth polypeptide has at least about 60% sequence identity to SEQ ID NO:93 or 95, or to a subsequence or fragment of at least about 20, 30, 40, 50, 60, 70, or more residues of SEQ ID NO: 93 or 95.

In some embodiments, the enzyme composition can further comprise a fifth polypeptide having GH61/endoglucanase activity, or alternatively, a GH61 endoglucanase-enriched whole cellulase. For example, the polypeptide having GH61/endoglucanase activity is an EGIV polypeptide from a suitable organism, such as a bacterium or a fungus, e.g., a T. reesei Eg4. In some embodiments, the fifth polypeptide, which is a GH61 endoglucanase polypeptide comprises at least about 60% (e.g., at least about 60%, 65%, 70%, 75%, 80%, 85%, 90%, 91%, 92%, 93%, 94%, 95%, 96%, 97%, 98%, or 99%) identity to any one of SEQ ID NOs: 52, 80-81, 206-207, over a region of at least about 10 (e.g., at least about 10, 15, 20, 25, 30, 35, 40, 45, 50, 55, 60, 65, 70, 75, 80, 85, 90, 95, 100, 125, 150, 175, 200, 225, 250, 275, 300) residues, or one that comprises one or more sequence motifs selected from the group consisting of: (1) SEQ ID NOs:84 and 88; (2) SEQ ID NOs:85 and 88; (3) SEQ ID NO:86; (4) SEQ ID NO:87; (5) SEQ ID NOs:84, 88 and 89; (6) SEQ ID NOs:85, 88, and 89; (7) SEQ ID NOs: 84, 88, and 90; (8) SEQ ID NOs: 85, 88 and 90; (9) SEQ ID NOs:84, 88 and 91; (10) SEQ ID NOs: 85, 88 and 91; (11) SEQ ID NOs: 84, 88, 89 and 91; (12) SEQ ID NOs: 84, 88, 90 and 91; (13) SEQ ID NOs: 85, 88, 89 and 91: and (14) SEQ ID NOs: 85, 88, 90 and 91. The enzyme composition can further comprise a cellobiose dehydrogenase. In certain embodiments, the first polypeptide having xylosidase activity is one selected from Group 1 β-xylosidase polypeptides. Group 1 β-xylosidase polypeptides have at least about 70% sequence identity to any one of SEQ ID NOs: 2 and 10, or to a mature sequences thereof. For example, Group β-xylosidase can be Fv3A or Fv43A.

In certain embodiments, the second polypeptide having xylosidase activity is one selected from Group 2 β-xylosidase polypeptides. Group 2 β-xylosidase polypeptides have at least about 70% sequence identity to any one of SEQ ID NOs:4, 6, 8, 10, 12, 14, 16, 18, 28, 30, and 45, or to a mature sequence thereof. For example, Group 2 β-xylosidases can be Pf43A, Fv43E, Fv39A, Fv43B, Pa51A, Gz43A, Fo43A, Fv43D, Pf43B, or T. reesei Bxl1.

In some embodiments, the third polypeptide having arabinofuranosidase activity has at least about 70% sequence identity to any one of SEQ ID NOs:12, 14, 20, 22, and 32, or to a mature sequence thereof. For example, the third polypeptide can be Fv43B, Pa51A, Af43A, Pf51A, or Fv51A.

The first, second, third, fourth, fifth or other polypeptide can be isolated or purified form a naturally-occurring source. Alternatively, it can be expressed or overexpressed by a recombinant host cell. It can be added to an enzyme composition in an isolated or purified form. It can be expressed or overexpressed by a host organism or host cell as a part of culture mixture, e.g., a fermentation broth. In some embodiments, a gene encoding such polypeptide can be integrated into the genetic material of the host organism, which allows the expression of the encoded polypeptides by that organism.

A fifth non-limiting example of an enzyme composition comprises (1) a first polypeptide having xylosidase activity, (2) a second polypeptide (different from the first) having xylosidase activity, and (3) a third polypeptide having arabinofuranosidase activity, and (4) a β-glucosidase enriched whole cellulase. In certain embodiments, the β-glucosidase enriched whole cellulase is enriched with a polypeptide that has at least about 60% (e.g., at least about 60%, 65%, 70%, 75%, 80%, 85%, 90%, 91%, 92%, 93%, 94%, 95%, 96%, 97%, 98%, or 99%) sequence identity to any one of SEQ ID NOs: 54, 56, 58, 60, 62, 64, 66, 68, 70, 72, 74, 76, 78, 79, 93, and 95, over a region of at least about 10 (e.g., at least about 10, 15, 20, 25, 30, 35, 40, 45, 50, 55, 60, 65, 70, 75, 80, 85, 90, 95, 100, 125, 150, 175, 200, 225, 250, 275, 300) residues. In certain embodiments, the β-glucosidase enriched whole cellulase is enriched with a chimeric/fusion β-glucosidase polypeptide comprising two or more β-glucosidase sequences, wherein the first sequence derived from a first β-glucosidase is at least about 200 amino acid residues in length and comprises one or more or all of the amino acid sequence motifs of SEQ ID NOs: 96-108, whereas the second sequence derived from a second β-glucosidase is at least about 50 amino acid residues in length and comprises one or more or all of the amino acid sequence motifs of SEQ ID NOs:109-116, and optionally also a third sequence of 3, 4, 5, 6, 7, 8, 9, 10, or 11 amino acid residues in length encoding a loop sequence derived from a third β-glucosidase having an amino acid sequence of FDRRSPG (SEQ ID NO:204), or of FD(R/K)YNIT (SEQ ID NO:205). For example, the β-glucosidase enriched whole cellulase is enriched with a polypeptide that comprises a first sequence having least about 60% sequence identity to an at least 200-residue stretch of Fv3C (SEQ ID NO:60), e.g., an at least 200-residue stretch from the N-terminus or from a residue near to the N-terminus of SEQ ID NO:60, and a second sequence having at least about 60% sequence identity to an at least 50-residue stretch of T. reesei Bgl3 (Tr3B, SEQ ID NO:64), e.g., an at least 50-residue stretch from the C-terminus or from a residue near to the C-terminus of SEQ ID NO:64. In certain embodiments, the β-glucosidase enriched whole cellulase is enriched with a polypeptide that further comprises a third sequence of about 3, 4, 5, 6, 7, 8, 9, 10, or 11 amino acid residues that is derived from a sequence of equal length from Te3A (SEQ ID NO:66), or from a sequence having an amino acid sequence of FDRRSPG (SEQ ID NO:204), or of FD(R/K)YNIT (SEQ ID NO:205). For example, the β-glucosidase enriched whole cellulase is enriched with a polypeptide having at least about 60% sequence identity to SEQ ID NO:93 or 95, or to a subsequence or fragment of at least about 20, 30, 40, 50, 60, 70, or more residues of SEQ ID NO: 93 or 95.

In certain embodiments, the enzyme composition can comprise a fourth polypeptide having GH61/endoglucanase activity, or alternatively, a GH61 endoglucanase-enriched whole cellulase. For example, the polypeptide having GH61/endoglucanase activity is an EGIV polypeptide from a suitable organism such as a bacterium or a fungus, e.g., a T. reesei Eg4.

In some embodiments, the fifth polypeptide, which is a GH61 endoglucanase polypeptide comprises at least about 60% (e.g., at least about 60%, 65%, 70%, 75%, 80%, 85%, 90%, 91%, 92%, 93%, 94%, 95%, 96%, 97%, 98%, or 99%) identity to any one of SEQ ID NOs: 52, 80-81, 206-207, over a region of at least about 10 (e.g., at least about 10, 15, 20, 25, 30, 35, 40, 45, 50, 55, 60, 65, 70, 75, 80, 85, 90, 95, 100, 125, 150, 175, 200, 225, 250, 275, 300) residues, or comprises one or more sequence motifs selected from the group consisting of: (1) SEQ ID NOs:84 and 88; (2) SEQ ID NOs:85 and 88; (3) SEQ ID NO:86; (4) SEQ ID NO:87; (5) SEQ ID NOs:84, 88 and 89; (6) SEQ ID NOs:85, 88, and 89; (7) SEQ ID NOs: 84, 88, and 90; (8) SEQ ID NOs: 85, 88 and 90; (9) SEQ ID NOs:84, 88 and 91; (10) SEQ ID NOs: 85, 88 and 91; (11) SEQ ID NOs: 84, 88, 89 and 91; (12) SEQ ID NOs: 84, 88, 90 and 91; (13) SEQ ID NOs: 85, 88, 89 and 91: and (14) SEQ ID NOs: 85, 88, 90 and 91.

The enzyme composition can further comprise a cellobiose dehydrogenase.

In certain embodiments, the first polypeptide having xylosidase activity is one selected from Group 1 β-xylosidase polypeptides. Group 1 β-xylosidase polypeptides have at least about 70% sequence identity to any one of SEQ ID NOs: 2 and 10, or to a mature sequences thereof. For example, Group β-xylosidase can be Fv3A or Fv43A.

In certain embodiments, the second polypeptide having xylosidase activity is one selected from Group 2 β-xylosidase polypeptides. Group 2 β-xylosidase polypeptides have at least about 70% sequence identity to any one of SEQ ID NOs:4, 6, 8, 10, 12, 14, 16, 18, 28, 30, and 45, or to a mature sequence thereof. For example, Group 2 β-xylosidases can be Pf43A, Fv43E, Fv39A, Fv43B, Pa51A, Gz43A, Fo43A, Fv43D, Pf43B, or T. reesei Bxl1.

In some embodiments, the third polypeptide having arabinofuranosidase activity has at least about 70% sequence identity to any one of SEQ ID NOs:12, 14, 20, 22, and 32, or to a mature sequence thereof. For example, the third polypeptide can be Fv43B, Pa51A, Af43A, Pf51A, or Fv51A.

The first, second, third, fourth or other polypeptide can be isolated or purified form a naturally-occurring source. Alternatively, it can be expressed or overexpressed by a recombinant host cell. It can be added to an enzyme composition in an isolated or purified form. It can be expressed or overexpressed by a host organism or host cell as a part of culture mixture, e.g., a fermentation broth. In some embodiments, a gene encoding such a polypeptide can be integrated into the genetic material of the host organism, which allows the expression of the encoded polypeptides by that organism.

A sixth non-limiting example of an engineered enzyme composition of the invention comprises (1) a first polypeptide having xylosidase activity, (2) a second polypeptide (which differs from the first polypeptide) having xylosidase activity, (3) and a third polypeptide having arabinofuranosidase activity; and (4) a fourth polypeptide having GH61/endoglucanase activity, or alternatively, an EGIV-enriched whole cellulase. For example, the polypeptide having GH61/endoglucanase activity is an EGIV polypeptide from a suitable organism such as a bacterium or a fungus, e.g., a T. reesei Eg4. In some embodiments, the fifth polypeptide, which is a GH61 endoglucanase polypeptide comprises at least about 60% (e.g., at least about 60%, 65%, 70%, 75%, 80%, 85%, 90%, 91%, 92%, 93%, 94%, 95%, 96%, 97%, 98%, or 99%) identity to any one of SEQ ID NOs: 52, 80-81, 206-207, over a region of at least about 10 (e.g., at least about 10, 15, 20, 25, 30, 35, 40, 45, 50, 55, 60, 65, 70, 75, 80, 85, 90, 95, 100, 125, 150, 175, 200, 225, 250, 275, 300) residues, or one that comprises one or more sequence motifs selected from the group consisting of: (1) SEQ ID NOs:84 and 88; (2) SEQ ID NOs:85 and 88; (3) SEQ ID NO:86; (4) SEQ ID NO:87; (5) SEQ ID NOs:84, 88 and 89; (6) SEQ ID NOs:85, 88, and 89; (7) SEQ ID NOs: 84, 88, and 90; (8) SEQ ID NOs: 85, 88 and 90; (9) SEQ ID NOs:84, 88 and 91; (10) SEQ ID NOs: 85, 88 and 91; (11) SEQ ID NOs: 84, 88, 89 and 91; (12) SEQ ID NOs: 84, 88, 90 and 91; (13) SEQ ID NOs: 85, 88, 89 and 91: and (14) SEQ ID NOs: 85, 88, 90 and 91. The enzyme composition can further comprise a cellobiose dehydrogenase.

In certain embodiments, the first polypeptide having xylosidase activity is one selected from Group 1 β-xylosidase polypeptides. Group 1 β-xylosidase polypeptides have at least about 70% sequence identity to any one of SEQ ID NOs: 2 and 10, or to a mature sequences thereof. For example, Group β-xylosidase can be Fv3A or Fv43A.

In certain embodiments, the second polypeptide having xylosidase activity is one selected from Group 2 β-xylosidase polypeptides. Group 2 β-xylosidase polypeptides have at least about 70% sequence identity to any one of SEQ ID NOs:4, 6, 8, 10, 12, 14, 16, 18, 28, 30, and 45, or to a mature sequence thereof. For example, Group 2 β-xylosidases can be Pf43A, Fv43E, Fv39A, Fv43B, Pa51A, Gz43A, Fo43A, Fv43D, Pf43B, or T. reesei Bxl1.

In some embodiments, the third polypeptide having arabinofuranosidase activity has at least about 70% sequence identity to any one of SEQ ID NOs:12, 14, 20, 22, and 32, or to a mature sequence thereof. For example, the third polypeptide can be Fv43B, Pa51A, Af43A, Pf51A, or Fv51A.

The first, second, third, fourth or other polypeptide can be isolated or purified form a naturally-occurring source. Alternatively, it can be expressed or overexpressed by a recombinant host cell. It can be added to an enzyme composition in an isolated or purified form. It can be expressed or overexpressed by a host organism or host cell as a part of culture mixture, e.g., a fermentation broth. In some embodiments, a gene encoding such a polypeptide can be integrated into the genetic material of the host organism, which allows the expression of the encoded polypeptides by that organism.

A seventh non-limiting example of an engineered enzyme composition of the invention comprises (1) a first polypeptide having xylanase activity, (2) a second polypeptide having xylosidase activity, (3) a third polypeptide (different from the second polypeptide) having xylosidase activity, and (4) a fourth polypeptide having β-glucosidase activity. In certain embodiments, the fourth polypeptide has at least about 60% (e.g., at least about 60%, 65%, 70%, 75%, 80%, 85%, 90%, 91%, 92%, 93%, 94%, 95%, 96%, 97%, 98%, or 99%) identity to any one of SEQ ID NOs: 54, 56, 58, 60, 62, 64, 66, 68, 70, 72, 74, 76, 78, 79, 93, and 95, over a region of at least about 10 (e.g., at least about 10, 15, 20, 25, 30, 35, 40, 45, 50, 55, 60, 65, 70, 75, 80, 85, 90, 95, 100, 125, 150, 175, 200, 225, 250, 275, 300) residues. In certain embodiments, the fourth polypeptide is a chimeric/fusion β-glucosidase polypeptide comprising two or more β-glucosidase sequences, wherein the first sequence derived from a first β-glucosidase is at least about 200 amino acid residues in length and comprises one or more or all of the amino acid sequence motifs of SEQ ID NOs: 96-108, whereas the second sequence derived from a second β-glucosidase is at least about 50 amino acid residues in length and comprises one or more or all of the amino acid sequence motifs of SEQ ID NOs:109-116, and optionally also a third sequence of 3, 4, 5, 6, 7, 8, 9, 10, or 11 amino acid residues in length encoding a loop sequence derived from a third β-glucosidase having an amino acid sequence of FDRRSPG (SEQ ID NO:204), or of FD(R/K)YNIT (SEQ ID NO:205). In particular, the first of the two or more β-glucosidase sequences is one that is at least about 200 amino acid residues in length and comprises at least 2 (e.g., at least 2, 3, 4, or all) of the amino acid sequence motifs of SEQ ID NOs: 197-202, and the second of the two or more β-glucosidase is at least 50 amino acid residues in length and comprises SEQ ID NO:203, and optionally also a third sequence of about 3, 4, 5, 6, 7, 8, 9, 10, or 11 amino acid residues in length and having an amino acid sequence of FDRRSPG (SEQ ID NO:204), or of FD(R/K)YNIT (SEQ ID NO:205), which is derived from a third β-glucosidase polypeptide different from the first or the second β-glucosidase polypeptide. For example, the fourth polypeptide comprises a first sequence having least about 60% sequence identity to an at least 200-residue stretch of Fv3C (SEQ ID NO:60), e.g., an at least 200-residue stretch from the N-terminus or from a residue near to the N-terminus of SEQ ID NO:60, and a second sequence having at least about 60% sequence identity to an at least 50-residue stretch of T. reesei Bgl3 (Tr3B, SEQ ID NO:64), e.g., an at least 50-residue stretch from the C-terminus or from a residue near to the C-terminus of SEQ ID NO:64. In certain embodiments, the fourth polypeptide further comprises a third sequence of about 3, 4, 5, 6, 7, 8, 9, 10, or 11 amino acid residues that is derived from a sequence of equal length from Te3A (SEQ ID NO:66), or have an amino acid sequence of FDRRSPG (SEQ ID NO:204), or of FD(R/K)YNIT (SEQ ID NO:205). For example, the fourth polypeptide comprises a sequence that has at least about 60% sequence identity to SEQ ID NO:93 or 95, or to a subsequence or fragment of at least about 20, 30, 40, 50, 60, 70, or more residues of SEQ ID NO: 93 or 95.

The enzyme composition can further comprise a fifth polypeptide having GH61/endoglucanase activity, or alternatively, a GH61 endoglucanase-enriched whole cellulase.

For example, the polypeptide having GH61/endoglucanase activity is an EGIV polypeptide from a suitable organism such as a bacterium or a fungus, e.g., a T. reesei Eg4. In some embodiments, the fifth polypeptide, which is a GH61 endoglucanase polypeptide comprises at least about 60% (e.g., at least about 60%, 65%, 70%, 75%, 80%, 85%, 90%, 91%, 92%, 93%, 94%, 95%, 96%, 97%, 98%, or 99%) identity to any one of SEQ ID NOs: 52, 80-81, 206-207, over a region of at least about 10 (e.g., at least about 10, 15, 20, 25, 30, 35, 40, 45, 50, 55, 60, 65, 70, 75, 80, 85, 90, 95, 100, 125, 150, 175, 200, 225, 250, 275, 300) residues, or one that comprises one or more sequence motifs selected from the group consisting of: (1) SEQ ID NOs:84 and 88; (2) SEQ ID NOs:85 and 88; (3) SEQ ID NO:86; (4) SEQ ID NO:87; (5) SEQ ID NOs:84, 88 and 89; (6) SEQ ID NOs:85, 88, and 89; (7) SEQ ID NOs: 84, 88, and 90; (8) SEQ ID NOs: 85, 88 and 90; (9) SEQ ID NOs:84, 88 and 91; (10) SEQ ID NOs: 85, 88 and 91; (11) SEQ ID NOs: 84, 88, 89 and 91; (12) SEQ ID NOs: 84, 88, 90 and 91; (13) SEQ ID NOs: 85, 88, 89 and 91: and (14) SEQ ID NOs: 85, 88, 90 and 91. The enzyme composition can further comprise a cellobiose dehydrogenase.

In some embodiments, the first polypeptide having xylanase activity has at least about 70% sequence identity to any one of SEQ ID NOs: 24, 26, 42, and 43, or to a mature sequence thereof. For example, the first polypeptide can be AfuXyn2, AfuXyn5, T. reesei Xyn3, or T. reesei Xyn2.

In certain embodiments, the second polypeptide having xylosidase activity is one selected from Group 1 β-xylosidase polypeptides. Group 1 β-xylosidase polypeptides have at least about 70% sequence identity to any one of SEQ ID NOs: 2 and 10, or to a mature sequences thereof. For example, Group β-xylosidase can be Fv3A or Fv43A.

In certain embodiments, the third polypeptide having xylosidase activity is one selected from Group 2 β-xylosidase polypeptides. Group 2 β-xylosidase polypeptides have at least about 70% sequence identity to any one of SEQ ID NOs:4, 6, 8, 10, 12, 14, 16, 18, 28, 30, and 45, or to a mature sequence thereof. For example, Group 2 β-xylosidases can be Pf43A, Fv43E, Fv39A, Fv43B, Pa51A, Gz43A, Fo43A, Fv43D, Pf43B, or T. reesei Bxl1.

The first, second, third, fourth, fifth or other polypeptide can be isolated or purified form a naturally-occurring source. Alternatively, it can be expressed or overexpressed by a recombinant host cell. It can be added to an enzyme composition in an isolated or purified form. It can be expressed or overexpressed by a host organism or host cell as a part of culture mixture, for example a fermentation broth. In some embodiments, a gene encoding such a polypeptide can be integrated into the genetic material of the host organism, which allows the expression of the encoded polypeptides by that organism.

An eighth non-limiting example of an engineered enzyme composition comprises (1) a first polypeptide having xylanase activity, (2) a second polypeptide having xylosidase activity, (3) a third polypeptide (different from the second polypeptide) having xylosidase activity, and a β-glucosidase enriched whole cellulase. In certain embodiments, the β-glucosidase enriched whole cellulase is enriched with a polypeptide having at least about 60% (e.g., at least about 60%, 65%, 70%, 75%, 80%, 85%, 90%, 91%, 92%, 93%, 94%, 95%, 96%, 97%, 98%, or 99%) identity to any one of SEQ ID NOs: 54, 56, 58, 60, 62, 64, 66, 68, 70, 72, 74, 76, 78, 79, 93, and 95, over a region of at least about 10 (e.g., at least about 10, 15, 20, 25, 30, 35, 40, 45, 50, 55, 60, 65, 70, 75, 80, 85, 90, 95, 100, 125, 150, 175, 200, 225, 250, 275, 300) residues. In certain embodiments, the β-glucosidase enriched whole cellulase is enriched with a chimeric/fusion β-glucosidase polypeptide comprising two or more β-glucosidase sequences, wherein the first sequence derived from a first β-glucosidase is at least about 200 amino acid residues in length and comprises one or more or all of the amino acid sequence motifs of SEQ ID NOs: 96-108, whereas the second sequence derived from a second β-glucosidase is at least about 50 amino acid residues in length and comprises one or more or all of the amino acid sequence motifs of SEQ ID NOs:109-116, and optionally also a third sequence of 3, 4, 5, 6, 7, 8, 9, 10, or 11 amino acid residues in length encoding a loop sequence derived from a third β-glucosidase, having an amino acid sequence of FDRRSPG (SEQ ID NO:204), or of FD(R/K)YNIT (SEQ ID NO:205). In particular, the first of the two or more β-glucosidase sequences is one that is at least about 200 amino acid residues in length and comprises at least 2 (e.g., at least 2, 3, 4, or all) of the amino acid sequence motifs of SEQ ID NOs: 197-202, and the second of the two or more β-glucosidase is at least 50 amino acid residues in length and comprises SEQ ID NO:203, and optionally also a third sequence of about 3, 4, 5, 6, 7, 8, 9, 10, or 11 amino acid residues in length and having an amino acid sequence of FDRRSPG (SEQ ID NO:204), or of FD(R/K)YNIT (SEQ ID NO:205), which is derived from a third β-glucosidase polypeptide different from the first or the second β-glucosidase polypeptide. For example, the β-glucosidase enriched whole cellulase is enriched with a polypeptide that comprises a first sequence having least about 60% sequence identity to an at least 200-residue stretch of Fv3C (SEQ ID NO:60), e.g., an at least 200-residue stretch from the N-terminus or from a residue near to the N-terminus of SEQ ID NO:60, and a second sequence having at least about 60% sequence identity to an at least 50-residue stretch of T. reesei Bgl3 (Tr3B, SEQ ID NO:64), e.g., an at least 50-residue stretch from the C-terminus or from a residue near to the C-terminus of SEQ ID NO:64. In some embodiments, the β-glucosidase enriched whole cellulase is enriched with a polypeptide further comprising a third sequence of about 3, 4, 5, 6, 7, 8, 9, 10, or 11 amino acid residues that is derived from a sequence of equal length from Te3A (SEQ ID NO:66), or have an amino acid sequence of FDRRSPG (SEQ ID NO:204), or of FD(R/K)YNIT (SEQ ID NO:205). For example, the β-glucosidase enriched whole cellulase is enriched with a polypeptide comprising a sequence having at least about 60% sequence identity to SEQ ID NO:93 or 95, or to a subsequence or fragment of at least about 20, 30, 40, 50, 60, 70, or more residues of SEQ ID NO: 93 or 95.

The enzyme composition can further comprise a fourth polypeptide having GH61/endoglucanase activity, or alternatively, a GH61 endoglucanase-enriched whole cellulase.

For example, the polypeptide having GH61/endoglucanase activity is an EGIV polypeptide from a suitable organism such as a bacterium or a fungus, e.g., a T. reesei Eg4. In some embodiments, the fourth polypeptide, which is a GH61 endoglucanase polypeptide, comprises at least about 60% (e.g., at least about 60%, 65%, 70%, 75%, 80%, 85%, 90%, 91%, 92%, 93%, 94%, 95%, 96%, 97%, 98%, or 99%) sequence identity to any one of SEQ ID NOs: 52, 80-81, 206-207, over a region of at least about 10 (e.g., at least about 10, 15, 20, 25, 30, 35, 40, 45, 50, 55, 60, 65, 70, 75, 80, 85, 90, 95, 100, 125, 150, 175, 200, 225, 250, 275, 300) residues, or one that comprises one or more sequence motifs selected from the group consisting of: (1) SEQ ID NOs:84 and 88; (2) SEQ ID NOs:85 and 88; (3) SEQ ID NO:86; (4) SEQ ID NO:87; (5) SEQ ID NOs:84, 88 and 89; (6) SEQ ID NOs:85, 88, and 89; (7) SEQ ID NOs: 84, 88, and 90; (8) SEQ ID NOs: 85, 88 and 90; (9) SEQ ID NOs:84, 88 and 91; (10) SEQ ID NOs: 85, 88 and 91; (11) SEQ ID NOs: 84, 88, 89 and 91; (12) SEQ ID NOs: 84, 88, 90 and 91; (13) SEQ ID NOs: 85, 88, 89 and 91: and (14) SEQ ID NOs: 85, 88, 90 and 91. The enzyme composition can further comprise a cellobiose dehydrogenase.

In some embodiments, the first polypeptide having xylanase activity has at least about 70% sequence identity to any one of SEQ ID NOs: 24, 26, 42, and 43, or to a mature sequence thereof. For example, the first polypeptide can be AfuXyn2, AfuXyn5, T. reesei Xyn3, or T. reesei Xyn2.

In certain embodiments, the second polypeptide having xylosidase activity is one selected from Group 1 β-xylosidase polypeptides. Group 1 β-xylosidase polypeptides have at least about 70% sequence identity to any one of SEQ ID NOs: 2 and 10, or to a mature sequences thereof. For example, Group β-xylosidase can be Fv3A or Fv43A.

In certain embodiments, the third polypeptide having xylosidase activity is one selected from Group 2 β-xylosidase polypeptides. Group 2 β-xylosidase polypeptides have at least about 70% sequence identity to any one of SEQ ID NOs:4, 6, 8, 10, 12, 14, 16, 18, 28, 30, and 45, or to a mature sequence thereof. For example, Group 2 β-xylosidases can be Pf43A, Fv43E, Fv39A, Fv43B, Pa51A, Gz43A, Fo43A, Fv43D, Pf43B, or T. reesei Bxl1.

The first, second, third, fourth, or other polypeptide can be isolated or purified form a naturally-occurring source. Alternatively, it can be expressed or overexpressed by a recombinant host cell. It can be added to an enzyme composition in an isolated or purified form. It can be expressed or overexpressed by a host organism or host cell as a part of culture mixture, for example a fermentation broth. In some embodiments, a gene encoding such a polypeptide can be integrated into the genetic material of the host organism, which allows the expression of the encoded polypeptides by that organism.

A ninth non-limiting example of an engineered enzyme composition comprises (1) a first polypeptide having xylanase activity, (2) a second polypeptide having xylosidase activity, (3) a third polypeptide (different from the second polypeptide) having xylosidase activity, (4) and a fourth polypeptide having GH61/endoglucanase activity, or alternatively a GH61 endoglucanase-enriched whole cellulase. In some embodiments, the fourth polypeptide having GH61/endoglucanase activity is an EGIV polypeptide from a suitable organism such as a bacterium or a fungus, e.g., a T. reesei Eg4. In some embodiments, the fifth polypeptide, which is a GH61 endoglucanase polypeptide, has at least about 60% (e.g., at least about 60%, 65%, 70%, 75%, 80%, 85%, 90%, 91%, 92%, 93%, 94%, 95%, 96%, 97%, 98%, or 99%) identity to any one of SEQ ID NOs: 52, 80-81, 206-207, over a region of at least about 10 (e.g., at least about 10, 15, 20, 25, 30, 35, 40, 45, 50, 55, 60, 65, 70, 75, 80, 85, 90, 95, 100, 125, 150, 175, 200, 225, 250, 275, 300) residues, or is one that comprises one or more sequence motifs selected from the group consisting of: (1) SEQ ID NOs:84 and 88; (2) SEQ ID NOs:85 and 88; (3) SEQ ID NO:86; (4) SEQ ID NO:87; (5) SEQ ID NOs:84, 88 and 89; (6) SEQ ID NOs:85, 88, and 89; (7) SEQ ID NOs: 84, 88, and 90; (8) SEQ ID NOs: 85, 88 and 90; (9) SEQ ID NOs:84, 88 and 91; (10) SEQ ID NOs: 85, 88 and 91; (11) SEQ ID NOs: 84, 88, 89 and 91; (12) SEQ ID NOs: 84, 88, 90 and 91; (13) SEQ ID NOs: 85, 88, 89 and 91: and (14) SEQ ID NOs: 85, 88, 90 and 91. The enzyme composition can further comprise a cellobiose dehydrogenase.

In some embodiments, the first polypeptide having xylanase activity has at least about 70% sequence identity to any one of SEQ ID NOs: 24, 26, 42, and 43, or to a mature sequence thereof. For example, the first polypeptide can be AfuXyn2, AfuXyn5, T. reesei Xyn3, or T. reesei Xyn2.

In certain embodiments, the second polypeptide having xylosidase activity is one selected from Group 1 β-xylosidase polypeptides. Group 1 β-xylosidase polypeptides have at least about 70% sequence identity to any one of SEQ ID NOs: 2 and 10, or to a mature sequences thereof. For example, Group β-xylosidase can be Fv3A or Fv43A.

In certain embodiments, the third polypeptide having xylosidase activity is one selected from Group 2 β-xylosidase polypeptides. Group 2 β-xylosidase polypeptides have at least about 70% sequence identity to any one of SEQ ID NOs:4, 6, 8, 10, 12, 14, 16, 18, 28, 30, and 45, or to a mature sequence thereof. For example, Group 2 β-xylosidases can be Pf43A, Fv43E, Fv39A, Fv43B, Pa51A, Gz43A, Fo43A, Fv43D, Pf43B, or T. reesei Bxl1.

The first, second, third, fourth or other polypeptide can be isolated or purified form a naturally-occurring source. Alternatively, it can be expressed or overexpressed by a recombinant host cell. It can be added to an enzyme composition in an isolated or purified form. It can be expressed or overexpressed by a host organism or host cell as a part of culture mixture, for example a fermentation broth. In some embodiments, a gene encoding such a polypeptide can be integrated into the genetic material of the host organism, which allows the expression of the encoded polypeptides by that organism.

A tenth non-limiting example of an engineered enzyme composition comprises (1) a first polypeptide having xylanase activity, (2) a second polypeptide having xylosidase activity, and (3) a third polypeptide having β-glucosidase activity. In certain embodiments, the third polypeptide has at least about 60% (e.g., at least about 60%, 65%, 70%, 75%, 80%, 85%, 90%, 91%, 92%, 93%, 94%, 95%, 96%, 97%, 98%, or 99%) identity to any one of SEQ ID NOs: 54, 56, 58, 60, 62, 64, 66, 68, 70, 72, 74, 76, 78, 79, 93, and 95, over a region of at least about 10 (e.g., at least about 10, 15, 20, 25, 30, 35, 40, 45, 50, 55, 60, 65, 70, 75, 80, 85, 90, 95, 100, 125, 150, 175, 200, 225, 250, 275, 300) residues. In certain embodiments, the third polypeptide is a chimeric/fusion β-glucosidase polypeptide comprising two or more β-glucosidase sequences, wherein the first sequence derived from a first β-glucosidase is at least about 200 amino acid residues in length and comprises one or more or all of the amino acid sequence motifs of SEQ ID NOs: 96-108, whereas the second sequence derived from a second β-glucosidase is at least about 50 amino acid residues in length and comprises one or more or all of the amino acid sequence motifs of SEQ ID NOs:109-116, and optionally also a third sequence of 3, 4, 5, 6, 7, 8, 9, 10, or 11 amino acid residues in length encoding a loop sequence derived from a third β-glucosidase, having an amino acid sequence of FDRRSPG (SEQ ID NO:204), or of FD(R/K)YNIT (SEQ ID NO:205). In particular, the first of the two or more β-glucosidase sequences is one that is at least about 200 amino acid residues in length and comprises at least 2 (e.g., at least 2, 3, 4, or all) of the amino acid sequence motifs of SEQ ID NOs: 197-202, and the second of the two or more β-glucosidase is at least 50 amino acid residues in length and comprises SEQ ID NO:203, and optionally also a third sequence of about 3, 4, 5, 6, 7, 8, 9, 10, or 11 amino acid residues in length and having an amino acid sequence of FDRRSPG (SEQ ID NO:204), or of FD(R/K)YNIT (SEQ ID NO:205), which is derived from a third β-glucosidase polypeptide different from the first or the second β-glucosidase polypeptide. For example, the third polypeptide comprises a first sequence having least about 60% sequence identity to an at least 200-residue stretch of Fv3C (SEQ ID NO:60), e.g., an at least 200-residue stretch from the N-terminus or from a residue near to the N-terminus of SEQ ID NO:60, and a second sequence having at least about 60% sequence identity to an at least 50-residue stretch of T. reesei Bgl3 (Tr3B, SEQ ID NO:64), e.g., an at least 50-residue stretch from the C-terminus or from a residue near to the C-terminus of SEQ ID NO:64. In certain embodiments, the third polypeptide further comprises a third sequence of about 3, 4, 5, 6, 7, 8, 9, 10, or 11 amino acid residues derived from a sequence of equal length from Te3A (SEQ ID NO:66), or comprises an amino acid sequence of FDRRSPG (SEQ ID NO:204), or of FD(R/K)YNIT (SEQ ID NO:205). For example, the third polypeptide comprises a sequence having at least about 60% sequence identity to SEQ ID NO:93 or 95, or to a subsequence or fragment of at least about 20, 30, 40, 50, 60, 70, or more residues of SEQ ID NO: 93 or 95.

The enzyme composition can further comprise a fourth polypeptide having GH61/endoglucanase activity, or alternatively, a GH61 endoglucanase-enriched whole cellulase.

For example, the polypeptide having GH61/endoglucanase activity is an EGIV polypeptide from a suitable organism such as a bacterium or a fungus, e.g., a T. reesei Eg4. In some embodiments, the fourth polypeptide, which is a GH61 endoglucanase polypeptide, has at least about 60% (e.g., at least about 60%, 65%, 70%, 75%, 80%, 85%, 90%, 91%, 92%, 93%, 94%, 95%, 96%, 97%, 98%, or 99%) identity to any one of SEQ ID NOs: 52, 80-81, 206-207, over a region of at least about 10 (e.g., at least about 10, 15, 20, 25, 30, 35, 40, 45, 50, 55, 60, 65, 70, 75, 80, 85, 90, 95, 100, 125, 150, 175, 200, 225, 250, 275, 300) residues, or comprises one or more sequence motifs selected from the group consisting of: (1) SEQ ID NOs:84 and 88; (2) SEQ ID NOs:85 and 88; (3) SEQ ID NO:86; (4) SEQ ID NO:87; (5) SEQ ID NOs:84, 88 and 89; (6) SEQ ID NOs:85, 88, and 89; (7) SEQ ID NOs: 84, 88, and 90; (8) SEQ ID NOs: 85, 88 and 90; (9) SEQ ID NOs:84, 88 and 91; (10) SEQ ID NOs: 85, 88 and 91; (11) SEQ ID NOs: 84, 88, 89 and 91; (12) SEQ ID NOs: 84, 88, 90 and 91; (13) SEQ ID NOs: 85, 88, 89 and 91: and (14) SEQ ID NOs: 85, 88, 90 and 91. The enzyme composition can further comprise a cellobiose dehydrogenase.

In some embodiments, the first polypeptide having xylanase activity has at least about 70% sequence identity to any one of SEQ ID NOs: 24, 26, 42, and 43, or to a mature sequence thereof. For example, the first polypeptide can be AfuXyn2, AfuXyn5, T. reesei Xyn3, or T. reesei Xyn2.

In some embodiments, the second polypeptide having xylosidase activity can be one selected from either a Group 1 or Group 2 β-xylosidase polypeptides. Group 1 β-xylosidase polypeptides have at least about 70% sequence identity to any one of SEQ ID NOs: 2 and 10, or to mature sequences thereof. For example, Group 1 β-xylosidase can be Fv3A or Fv43A. Group 2 β-xylosidase polypeptides have at least about 70% sequence identity to any one of SEQ ID NOs:4, 6, 8, 10, 12, 14, 16, 18, 28, 30, and 45, or to a mature sequence thereof. For example, Group 2 β-xylosidases can be Pf43A, Fv43E, Fv39A, Fv43B, Pa51A, Gz43A, Fo43A, Fv43D, Pf43B, or T. reesei Bxl1.

The first, second, third, fourth or other polypeptide can be isolated or purified form a naturally-occurring source. Alternatively, it can be expressed or overexpressed by a recombinant host cell. It can be added to an enzyme composition in an isolated or purified form. It can be expressed or overexpressed by a host organism or host cell as a part of culture mixture, for example a fermentation broth. In some embodiments, a gene encoding such a polypeptide can be integrated into the genetic material of the host organism, which allows the expression of the encoded polypeptides by that organism.

An eleventh non-limiting example of an engineered enzyme composition comprises (1) a first polypeptide having xylanase activity, (2) a second polypeptide having xylosidase activity, and a β-glucosidase enriched whole cellulase. In some embodiments, the β-glucosidase enriched whole cellulase is enriched with a polypeptide that has at least about 60% (e.g., at least about 60%, 65%, 70%, 75%, 80%, 85%, 90%, 91%, 92%, 93%, 94%, 95%, 96%, 97%, 98%, or 99%) identity to any one of SEQ ID NOs: 54, 56, 58, 60, 62, 64, 66, 68, 70, 72, 74, 76, 78, 79, 93, and 95, over a region of at least about 10 (e.g., at least about 10, 15, 20, 25, 30, 35, 40, 45, 50, 55, 60, 65, 70, 75, 80, 85, 90, 95, 100, 125, 150, 175, 200, 225, 250, 275, 300) residues. In certain embodiments, the β-glucosidase enriched whole cellulase is enriched with a chimeric/fusion β-glucosidase polypeptide comprising two or more β-glucosidase sequences, wherein the first sequence derived from a first β-glucosidase is at least about 200 amino acid residues in length and comprises one or more or all of the amino acid sequence motifs of SEQ ID NOs: 96-108, whereas the second sequence derived from a second β-glucosidase is at least about 50 amino acid residues in length and comprises one or more or all of the amino acid sequence motifs of SEQ ID NOs:109-116, and optionally also a third sequence of 3, 4, 5, 6, 7, 8, 9, 10, or 11 amino acid residues in length encoding a loop sequence derived from a third β-glucosidase, having an amino acid sequence of FDRRSPG (SEQ ID NO:204), or of FD(R/K)YNIT (SEQ ID NO:205). In particular, the first of the two or more β-glucosidase sequences is one that is at least about 200 amino acid residues in length and comprises at least 2 (e.g., at least 2, 3, 4, or all) of the amino acid sequence motifs of SEQ ID NOs: 197-202, and the second of the two or more β-glucosidase is at least 50 amino acid residues in length and comprises SEQ ID NO:203, and optionally also a third sequence of about 3, 4, 5, 6, 7, 8, 9, 10, or 11 amino acid residues in length and having an amino acid sequence of FDRRSPG (SEQ ID NO:204), or of FD(R/K)YNIT (SEQ ID NO:205), which is derived from a third β-glucosidase polypeptide different from the first or the second β-glucosidase polypeptide. For example, the β-glucosidase enriched whole cellulase is enriched with a polypeptide that comprises a first sequence having least about 60% sequence identity to an at least 200-residue stretch of Fv3C (SEQ ID NO:60), e.g, an at least 200-residue stretch from the N-terminus or from a residue near to the N-terminus of SEQ ID NO:60, and a second sequence having at least about 60% sequence identity to an at least 50-residue stretch of T. reesei Bgl3 (Tr3B, SEQ ID NO:64), e.g., an at least 50-residue stretch from the C-terminus or from a residue near to the C-terminus of SEQ ID NO:64. In some embodiments, the β-glucosidase enriched whole cellulase is enriched with a polypeptide further comprising a third sequence of about 3, 4, 5, 6, 7, 8, 9, 10, or 11 amino acid residues derived from a sequence of equal length from Te3A (SEQ ID NO:66), or comprises an amino acid sequence of FDRRSPG (SEQ ID NO:204), or of FD(R/K)YNIT (SEQ ID NO:205). For example, the β-glucosidase enriched whole cellulase is enriched with a polypeptide comprising a sequence having at least about 60% sequence identity to SEQ ID NO:93 or 95, or to a subsequence or fragment of at least about 20, 30, 40, 50, 60, 70, or more residues of SEQ ID NO: 93 or 95.

The enzyme composition can further comprise a third polypeptide having GH61/endoglucanase activity, or alternatively, a GH61 endoglucanase-enriched whole cellulase.

For example, the polypeptide having GH61/endoglucanase activity is an EGIV polypeptide from a suitable organism such as a bacterium or a fungus, e.g., a T. reesei Eg4. In some embodiments, the third polypeptide, which is a GH61 endoglucanase polypeptide, has at least about 60% (e.g., at least about 60%, 65%, 70%, 75%, 80%, 85%, 90%, 91%, 92%, 93%, 94%, 95%, 96%, 97%, 98%, or 99%) identity to any one of SEQ ID NOs: 52, 80-81, 206-207, over a region of at least about 10 (e.g., at least about 10, 15, 20, 25, 30, 35, 40, 45, 50, 55, 60, 65, 70, 75, 80, 85, 90, 95, 100, 125, 150, 175, 200, 225, 250, 275, 300) residues, or comprises one or more sequence motifs selected from the group consisting of: (1) SEQ ID NOs:84 and 88; (2) SEQ ID NOs:85 and 88; (3) SEQ ID NO:86; (4) SEQ ID NO:87; (5) SEQ ID NOs:84, 88 and 89; (6) SEQ ID NOs:85, 88, and 89; (7) SEQ ID NOs: 84, 88, and 90; (8) SEQ ID NOs: 85, 88 and 90; (9) SEQ ID NOs:84, 88 and 91; (10) SEQ ID NOs: 85, 88 and 91; (11) SEQ ID NOs: 84, 88, 89 and 91; (12) SEQ ID NOs: 84, 88, 90 and 91; (13) SEQ ID NOs: 85, 88, 89 and 91: and (14) SEQ ID NOs: 85, 88, 90 and 91. The enzyme composition can further comprise a cellobiose dehydrogenase.

In some embodiments, the first polypeptide having xylanase activity has at least about 70% sequence identity to any one of SEQ ID NOs: 24, 26, 42, and 43, or to a mature sequence thereof. For example, the first polypeptide can be AfuXyn2, AfuXyn5, T. reesei Xyn3, or T. reesei Xyn2.

In some embodiments, the second polypeptide having xylosidase activity can be one selected from either a Group 1 or Group 2 β-xylosidase polypeptides. Group 1 β-xylosidase polypeptides have at least about 70% sequence identity to any one of SEQ ID NOs: 2 and 10, or to mature sequences thereof. For example, Group 1 β-xylosidase can be Fv3A or Fv43A. Group 2 β-xylosidase polypeptides have at least about 70% sequence identity to any one of SEQ ID NOs:4, 6, 8, 10, 12, 14, 16, 18, 28, 30, and 45, or to a mature sequence thereof. For example, Group 2 β-xylosidases can be Pf43A, Fv43E, Fv39A, Fv43B, Pa51A, Gz43A, Fo43A, Fv43D, Pf43B, or T. reesei Bxl1.

The first, second or other polypeptide can be isolated or purified form a naturally-occurring source. Alternatively, it can be expressed or overexpressed by a recombinant host cell. It can be added to an enzyme composition in an isolated or purified form. It can be expressed or overexpressed by a host organism or host cell as a part of culture mixture, for example a fermentation broth. In some embodiments, a gene encoding such a polypeptide can be integrated into the genetic material of the host organism, which allows the expression of the encoded polypeptides by that organism.

A twelfth non-limiting example of an engineered enzyme composition comprises (1) a first polypeptide having xylanase activity, (2) a second polypeptide having xylosidase activity, and (3) a third polypeptide having GH61/endoglucanase activity, or alternatively, a GH61 endoglucanase-enriched whole cellulase. In some embodiments, the polypeptide having GH61/endoglucanase activity is an EGIV polypeptide from a suitable organism such as a bacterium or a fungus, e.g., a T. reesei Eg4. In some embodiments, the third polypeptide, which is a GH61 endoglucanase polypeptide, has at least about 60% (e.g., at least about 60%, 65%, 70%, 75%, 80%, 85%, 90%, 91%, 92%, 93%, 94%, 95%, 96%, 97%, 98%, or 99%) identity to any one of SEQ ID NOs: 52, 80-81, 206-207, over a region of at least about 10 (e.g., at least about 10, 15, 20, 25, 30, 35, 40, 45, 50, 55, 60, 65, 70, 75, 80, 85, 90, 95, 100, 125, 150, 175, 200, 225, 250, 275, 300) residues, or comprises one or more sequence motifs selected from the group consisting of: (1) SEQ ID NOs:84 and 88; (2) SEQ ID NOs:85 and 88; (3) SEQ ID NO:86; (4) SEQ ID NO:87; (5) SEQ ID NOs:84, 88 and 89; (6) SEQ ID NOs:85, 88, and 89; (7) SEQ ID NOs: 84, 88, and 90; (8) SEQ ID NOs: 85, 88 and 90; (9) SEQ ID NOs:84, 88 and 91; (10) SEQ ID NOs: 85, 88 and 91; (11) SEQ ID NOs: 84, 88, 89 and 91; (12) SEQ ID NOs: 84, 88, 90 and 91; (13) SEQ ID NOs: 85, 88, 89 and 91: and (14) SEQ ID NOs: 85, 88, 90 and 91. The enzyme composition can further comprise a cellobiose dehydrogenase.

In some embodiments, the first polypeptide having xylanase activity has at least about 70% sequence identity to any one of SEQ ID NOs: 24, 26, 42, and 43, or to a mature sequence thereof. For example, the first polypeptide can be AfuXyn2, AfuXyn5, T. reesei Xyn3, or T. reesei Xyn2.

In some embodiments, the second polypeptide having xylosidase activity can be one selected from either a Group 1 or Group 2 β-xylosidase polypeptides. Group 1 β-xylosidase polypeptides have at least about 70% sequence identity to any one of SEQ ID NOs: 2 and 10, or to mature sequences thereof. For example, Group 1 β-xylosidase can be Fv3A or Fv43A. Group 2 β-xylosidase polypeptides have at least about 70% sequence identity to any one of SEQ ID NOs:4, 6, 8, 10, 12, 14, 16, 18, 28, 30, and 45, or to a mature sequence thereof. For example, Group 2 β-xylosidases can be Pf43A, Fv43E, Fv39A, Fv43B, Pa51A, Gz43A, Fo43A, Fv43D, Pf43B, or T. reesei Bxl1.

The first, second, third or other polypeptide can be isolated or purified form a naturally-occurring source. Alternatively, it can be expressed or overexpressed by a recombinant host cell. It can be added to an enzyme composition in an isolated or purified form. It can be expressed or overexpressed by a host organism or host cell as a part of culture mixture, for example a fermentation broth. In some embodiments, a gene encoding such a polypeptide can be integrated into the genetic material of the host organism, which allows the expression of the encoded polypeptides by that organism.

The engineered enzyme composition described herein is, for example, a fermentation broth. The fermentation broth is, e.g., one obtained from a microorganism. The microorganism can be a bacterium or a fungus such as a filamentous fungus or yeast. Suitable filamentous fungus include, without limitation, a Trichoderma, Humicola, Fusarium, Aspergillus, Neurospora, Penicillium, Cephalosporium, Achlya, Podospora, Endothia, Mucor, Cochliobolus, Pyricularia, or Chrysosporium. An example of a suitable fungus of Trichoderma spp. is Trichoderma reesei. An example of a suitable fungus of Penicillium spp. is Penicillium funiculosum. The fermentation broth can be, e.g., a cell-free fermentation broth or a whole broth formulation.

The enzyme composition described herein, when comprising an enzyme having cellulase activity, e.g., a cellobiohydrolase activity, an endoglucanase activity, a GH61/endoglucanase activity, or a β-glucosidase activity, or when comprising a whole cellulase, is a cellulase composition. The cellulase composition can be, e.g., a bacterial or fungal cellulase composition. For example, a filamentous fungal cellulase composition can be a Trichoderma, Aspergillus, or Chrysosporium such as a Trichoderma reesei, Aspergillus niger, Aspergillus oryzae, or Chrysosporium lucknowence cellulase composition. The cellulase composition can suitably be produced by a filamentous fungus, for example, by a Trichoderma, such as a Trichoderma reesei, by an Aspergillus, such as an Aspergillus niger or Aspergillus oryzae, or by a Chrysosporium, such as a Chrysosporium lucknowence. The enzyme composition can alternatively be produced in a recombinant organism such as a yeast.

The components of the enzyme compositions herein can be measured using known methods in the art. For example, SDS-PAGE can be used to measure the relative amounts of components although such measurements are not precise and are at best semi-quantitative. HPLC is typically deemed a more precise measurement of enzymatic components, although even its accuracy often depends on the availability of good enzyme standards to which the measured amounts can be combined, and the cleanliness of the mixture, as well as the capacity of the columns used to resolve certain co-eluting components. The components can also be measured using ultra performance liquid chromatography (UPLC), which, like HPLC, has limitations in resolve certain proteins from each other, but tends to have these limitations with regard to a different set of proteins. Thus, proteins that do not resolve using HPLC can sometimes be resolved using UPLC, and vise versa. The conditions used for measurements with these methods are described herein in the examples. The combined weight of polypeptide(s) having xylanase activity in the engineered composition, as measured by any of the SDS-PAGE, HPLC, or UPLC, can represent about 0.05 wt. % to about 80 wt. % (e.g., about 0.05 wt. % to about 75 wt. %, about 0.1 wt. % to about 70 wt. %, about 1 wt. % to about 60 wt. %, about 5 wt. % to about 50 wt. %, about 10 wt. % to about 40 wt. %, about 0.5 wt. % to about 40 wt. %, about 1 wt. % to about 35 wt. %, about 5 wt. % to about 25 wt. %, about 9 wt. % to about 17 wt. %, about 5 wt. % to about 15 wt. %, about 10 wt. % to about 15 wt. %, about 10 wt. % to about 25 wt. %, about 10 wt. % to about 35 wt. %, etc) of the combined or total protein weight in the enzyme composition. In a particular example, the combined weight of polypeptide(s) having xylanase activity is measured by the amount of T. reesei Xyn2 and T. reesei Xyn3, in a composition comprising these xylanases, e.g., any of the engineered enzyme compositions described herein. The amount of total weight of xylanases in that mixture is about 10 wt. % to about 20 wt. %, or about 14 wt. % to about 18 wt. % of the total weight of proteins in the composition, as measured using SDS-PAGE, HPLC, or UPLC using the methods described herein.

The combined weight of polypeptide(s) having β-xylosidase activity as measured by SDS-PAGE, HPLC or UPLC, can constitute about 0.05 wt. % to about 75 wt. % (e.g., about 0.05 wt. % to about 70 wt. %, about 0.1 wt. % to about 60 wt. %, about 1 wt. % to about 50 wt. %, about 10 wt. % to about 40 wt. %, about 20 wt. % to about 30 wt. %, about 2 wt. % to about 45 wt %, about 5 wt. % to about 40 wt. %, about 10 wt. % to about 35 wt. %, about 2 wt. % to about 30 wt. %, about 5 wt. % to about 25 wt. %, about 5 wt. % to about 10 wt. %, about 9 wt. % to about 15 wt. %, about 10 wt. % to about 20 wt. %, etc) of the total proteins in the engineered enzyme composition. In a particular example, the combined weight of polypeptide(s) having β-xylosidase activity is measured by the amount of a Group 1 β-xylosidase and a Group 2 β-xylosidase, e.g., Fv3A and Fv43D, in a composition comprising those β-xylosidases, e.g., any of the engineered enzyme compositions herein. The amount of total weight of β-xylosidases in that mixture is about 3 wt. % to about 20 wt. %, for example about 4 wt. % to about 6 wt. % as measured using HPLC, about 10 wt. % to about 14 wt. % as measured using UPLC, and about 15 wt. % to about 18 wt. % as measured using SDS-PAGE, in accordance with the methods described herein.

When an engineered enzyme composition of the invention comprises a Group 1 polypeptide having β-xylosidase activity and a Group 2 polypeptide having β-xylosidase activity, the combined weight of Group 1 polypeptide(s) can constitute about 0.1 wt. % to about 30 wt. % (e.g., about 0.2 wt. % to about 25 wt. %, about 0.5 wt. % to about 20 wt. %, about 4 wt. % to about 10 wt. %, about 4 wt. % to about 8 wt. %, etc) of the total protein weight in the composition, whereas the combined weight of the Group 2 polypeptide(s) can constitute about 0.1 wt. % to 20 wt. % (e.g., about 0.2 wt. % to about 18 wt. %, about 0.5 wt. % to about 15 wt. %, about 5 wt. % to about 10 wt. %, etc.) of the total protein weight in the composition.

The ratio of the weight of Group 1 β-xylosidase polypeptide(s) to that of Group 2 β-xylosidase polypeptide(s) can be, about 1:10 to about 10:1, e.g., about 1:8 to about 8:1, about 1:6 to about 6:1, about 1:4 to about 4:1, about 1:2 to about 2:1, or about 1:1.

The combined weight of polypeptide(s) having L-α-arabinofuranosidase activity, if present, can constitute about 0.05 wt. % to about 20 wt. % (e.g., 0.1 wt. % to about 15 wt. %, 1 wt. % to about 10 wt. %, 2 wt. % to about 12 wt. %, 4 wt. % to about 10 wt. %, 3 wt. % to about 9 wt. %, 5 wt. % to about 9 wt. %, etc) of the combined or total protein weight in the engineered enzyme composition, as measured using SDS-PAGE, HPLC, or UPLC. The combined weight of polypeptide(s) having L-α-arabinofuranosidase activity is, e.g., measured by the amount of Fv51A, in a composition comprising this L-α-arabinofuranosidase, e.g., any of the engineered enzyme compositions herein. The amount of total weight of L-α-arabinofuranosidase in that mixture is about 0.2 wt. % to about 2 wt. %, for example about 0.3 wt. % to about 0.5 wt. % as measured using HPLC, about 0.8 wt. % to about 1.2 wt. % as measured using UPLC and SDS-PAGE, in accordance with the methods described herein.

The combined weight of polypeptide(s) having β-glucosidase activity (including variants, mutants, or chimeric/fusion β-glucosidase polypeptides) can constitute about 0.05 wt. % to about 50 wt. % (e.g., about 0.1 wt. % to about 45 wt. %, about 1 wt. % to about 42 wt. %, about 2 wt. % to about 45 wt. %, about 2 wt. % to about 40 wt. %, about 2 wt. % to about 30 wt. %, about 2 wt. % to about 25 wt. %, about 5 wt. % to about 50 wt. %, about 9 wt. % to about 17 wt. %, about 10 wt. % to about 50 wt. %, about 20 wt. % to about 50 wt. %, about 25 wt. % to about 50 wt. %, about 30 wt. % to about 50 wt. %, etc) of the combined or total protein weight in the engineered enzyme composition, as measured using SDS-PAGE, UPLC or HPLC. In a particular example, the combined weight of polypeptide(s) having β-glucosidase activity is measured by the amount of a β-glucosidase hybrid/chimera of, e.g., SEQ ID NO:92, and T. reesei Bgl1, in a composition comprising such enzymes, e.g., any of the engineered enzyme compositions herein. The amount of total weight of β-glucosidase in that mixture is about 18 wt. % to about 28 wt. %, for example about 22 wt. % to about 25 wt. % if measured by SDS-PAGE and UPLC, and about 18 wt. % to about 22 wt. % if measured using HPLC in accordance with the methods described herein.

The total weight of the GH61 endoglucanase polypeptides can represent or constitute about 2 wt. % to about 50 wt. % (e.g., about 2 wt. % to about 45 wt. %, about 2 wt. % to about 40 wt. %, about 2 wt. % to about 30 wt. %, about 2 wt. % to about 25 wt. %, about 4 wt. % to about 16 wt. %, about 5 wt. % to about 50 wt. %, about 10 wt. % to about 50 wt. %, about 20 wt. % to about 50 wt. %, about 25 wt. % to about 50 wt. %, about 30 wt. % to about 50 wt. %, etc) of the combined or total protein weight in the engineered enzyme composition as measured by SDS-PAGE, HPLC or UPLC. In a particular example, the combined weight of polypeptide(s) having GH61/endoglucanase activity is measured by the amount of a T. reesei Eg4 polypeptide, in a composition comprising such enzymes, e.g., any of the engineered enzyme compositions herein. The amount of total weight of T. reesei Eg4 in that mixture is about 6 wt. % to about 20 wt. %, for example about 6 wt. % to about 10 wt. % if measured by HPLC, and about 6 wt. % to about 18 wt. % if measured using UPLC or SDS-PAGE in accordance with the methods described herein.

An example of an engineered enzyme composition of the invention comprises, in accordance with an HPLC measurement using conditions described in the examples herein, about 4 wt. % to about 6 wt. % of a Group 1 β-xylosidase polypeptide, about 5 wt. % to about 9 wt. % of a combined weight of a Group 2 β-xylosidase polypeptide and an L-α-arabinofuranosidase polypeptide, about 9 wt. % to about 17 wt. % of a β-glucosidase polypeptide, about 9 wt. % to about 17 wt. % of a xylanase, about 4 wt. % to about 16 wt. % of a GH61 endoglucanase. The enzyme composition can further comprise about 25 wt. % to about 45 wt. % of one or more cellobiohydrolase(s). The enzyme composition can also comprise about 7 wt. % to about 20 wt. % of other cellulases.

An example of an engineered enzyme composition of the invention comprises, in accordance with a UPLC measurement using conditions described in the examples herein about 4 wt. % to about 6 wt. % of a Group 1 β-xylosidase polypeptide, about 5 wt. % to about 9 wt. % of a Group 2 β-xylosidase polypeptide, about 0.5 wt. % to about 2 wt. % of an L-α-arabinofuranosidase polypeptide, about 18 wt. % to about 22 wt. % of β-glucosidase polypeptides, about 13 wt. % to about 15 wt. % of xylanase polypeptides, and about 8 wt. % to about 20 wt. % of a GH61 endoglucanase. The enzyme composition can further comprise about 15 wt. % to about 25 wt. % of cellobiohydrolases, e.g., T. reesei CBH1 and CBH2. The enzyme composition may further comprise about 2 wt. % to about 8 wt. % of other cellulases.

At least one (e.g., one or more, two or more, three or more, four or more, five or more, or even six or more) enzyme in an engineered enzyme composition of the invention is derived from a heterologous biological source, such as, for example, a microorganism, that is different from the host cell. In a non-limiting example, one of the enzymes in an engineered enzyme composition is from a filamentous fungus of the Fusarium spp., whereas the engineered enzyme composition is produced by a microorganism that is not a Fusarium spp., fungus. In another example, one of the enzymes in an engineered enzyme composition is from a filamentous fungus of the Trichoderma spp., whereas the engineered enzyme composition is produced by a microorganism that is not a Trichoderma spp. fungus, for example, an Aspergillus or Chrysosporium.

At least two enzymes in the engineered enzyme composition described herein are derived from different biological sources. In an exemplary engineered enzyme composition, one or more enzymes are derived from a Fusarium spp., whereas one or more other enzymes are derived from a fungus that is not a Fusarium spp.

The engineered enzyme composition is, e.g., suitably a fermentation broth composition. The fermentation broth is, e.g., one of a filamentous fungus, including, without limitation, a Trichoderma, Humicola, Fusarium, Aspergillus, Neurospora, Penicillium, Cephalosporium, Achlya, Podospora, Endothia, Mucor, Cochliobolus, Pyricularia, or Chrysosporium. An example of a fungus of Trichoderma spp. is Trichoderma reesei. An example of a fungus of Penicillium spp. is Penicillium funiculosum. An example of a fungus of Aspergilllus spp. is Aspergillus niger or Aspergillus oryzae. An example of a fungus of Chrysosporium spp. is Chrysosporium lucknowence. The fermentation broth can be, e.g., a cell-free fermentation broth, optionally subject to minimum post-production processing including, e.g., ultrafiltration, purification, cell kill, etc., and as such can be used in a whole broth formulation.

The engineered enzyme composition can also be a cellulase composition, e.g., a fungal cellulase composition or a bacterial cellulase composition. The cellulase composition, e.g., can be produced by a filamentous fungus, such as by a Trichoderma, an Aspergillus, a Chrysosporium, by a yeast, such as by Saccharomyces cerevisiae.

The enzymes or engineered enzyme compositions of the disclosure can be used in the food industry, e.g., for baking, for fruit and vegetable processing, in breaking down of agricultural waste, in the manufacture of animal feed, in pulp and paper production, in textile manufacture, or in household and industrial cleaning agents. The enzymes herein can be, e.g., each independently produced by a microorganism, such as a fungus or a bacterium.

The enzymes or engineered enzyme compositions herein can also be used to digest lignocellulose from any suitable sources, including all biological sources, such as plant biomasses, e.g., corn, grains, grasses (e.g., Indian grass, such as Sorghastrum nutans; or, switchgrass, e.g., Panicum species, such as Panicum virgatum), perennial canes (e.g., giant weeds), or, woods or wood processing byproducts, e.g., in the wood processing, pulp and/or paper industry, in textile manufacture, in household and industrial cleaning agents, and/or in biomass waste processing. The disclosure provides methods for hydrolyzing, breaking up, or disrupting a cellooligosaccharide, an arabinoxylan oligomer, or a glucan- or cellulose-comprising composition comprising contacting the composition with an enzyme or enzyme composition of the disclosure under suitable conditions, wherein the enzyme or the enzyme composition hydrolyzes, breaks up or disrupts the cellooligosaccharide, arabinoxylan oligomer, or glucan- or cellulose-comprising composition.

The disclosure provides engineered enzyme compositions comprising a polypeptide herein, or a polypeptide encoded by a nucleic acid herein. In some embodiments, the polypeptide has one or more activities selected from xylanase, xylosidase, L-α-arabinofuranosidase, glucosidase, and/or GH61/endoglucanase activities. The engineered enzyme compositions are used or are useful, for de-polymerization of cellulosic and hemicellulosic polymers into metabolizable carbon moieties. The engineered enzyme composition is suitably in the form of, e.g., a product of manufacture. The composition can be, e.g., a formulation, and can take the physical form of, e.g., a liquid or a solid.

An engineered enzyme composition herein can further optionally include a cellulase, e.g., a whole cellulase, comprising at least three different enzyme types selected from (1) an endoglucanase, (2) a cellobiohydrolase, and (3) a β-glucosidase; or at least three different enzymatic activities selected from (1) an endoglucanase activity catalyzing the cleavage of internal β-1,4 linkages of cellulosic or hemicellulosic materials, resulting in shorter glucooligosaccharides, (2) a cellobiohydrolase activity catalyzing the cleavage and release, in an “exo” manner, of cellobiose units (e.g., β-1,4 glucose-glucose disaccharide), and (3) a β-glucosidase activity catalyzing the release of glucose monomers from short cellooligosaccharides (e.g., cellobiose). The whole cellulase can be enriched with one or more β-glucosidase polypeptides. The whole cellulase can, in certain embodiments, be enriched with a GH61 endoglucanase polypeptide, e.g., an EGIV polypeptide, such as T. reesei Eg4. In certain embodiments, the whole cellulase can be enriched with a β-glucosidase polypeptide and a GH61 endoglucanase polypeptide. Engineered enzyme compositions of the disclosure are further described in Section 5.3. below.

In another aspect, the disclosure provides methods for processing a biomass material comprising contacting a composition comprising lignocellulose and/or a fermentable sugar with an enzyme herein, or with a polypeptide encoded by a nucleic acid herein, or with an engineered enzyme composition (e.g., a product of manufacture or a formula) herein.

Suitable biomass material comprising lignocellulose can be derived from, e.g., an agricultural crop, a byproduct of a food or feed production, a lignocellulosic waste product, a plant residue, or a waste paper or waste paper product. The polypeptides can suitably have one or more enzymatic activities selected from cellulase, endoglucanase, cellobiohydrolase, β-glucosidase, xylanase, mannanase, β-xylosidase, arabinofuranosidase, and other hemicellulase activities. Suitable plant residue can comprise grain, seeds, stems, leaves, hulls, husks, corncobs, corn stover, straw, grasses, canes, reeds, wood, wood chips, wood pulp and sawdust. The grasses can be, e.g., Indian grass or switchgrass. The reeds can be, e.g., perennial canes such as giant reeds. The paper waste can be, e.g., discarded or used photocopy paper, computer printer paper, notebook paper, notepad paper, typewriter paper, newspapers, magazines, cardboard, and paper-based packaging materials.

The disclosure provides compositions (including enzymes or engineered enzyme compositions, e.g., products of manufacture or a formula) comprising a mixture of hemicellulose- and cellulose-hydrolyzing enzymes, and at least one biomass material.

Optionally the biomass material comprises a lignocellulosic material derived from an agricultural crop, or is a byproduct of a food or feed production. Suitable biomass material can also be a lignocellulosic waste product, a plant residue, a waste paper or waste paper product, or comprises a plant residue. The plant residue can, e.g., be one comprising grains, seeds, stems, leaves, hulls, husks, corncobs, corn stover, grasses, straw, reeds, wood, wood chips, wood pulp, or sawdust. Exemplary grasses include, without limitation, Indian grass or switchgrass. Exemplary reeds include, without limitation, certain perennial canes such as giant reeds. Exemplary paper waste include, without limitation, discarded or used photocopy paper, computer printer paper, notebook paper, notepad paper, typewriter paper, newspapers, magazines, cardboard and paper-based packaging materials. Thus, the present disclosure provides compositions (including enzymes or engineered enzyme compositions, e.g., products of manufacture or a formula) that are useful for hydrolyzing hemicellulosic materials, catalyzing the enzymatic conversion of suitable biomass substrates to fermentable sugars. The present disclosure also provides methods of preparing such compositions as well as methods of using or applying such compositions in a research setting, an industrial setting, or in a commercial setting.

All publically available information as of the filing date, including, e.g., publications, patents, patent applications, GenBank sequences, and ATCC deposits cited herein are hereby expressly incorporated by reference.

### 4. BRIEF DESCRIPTION OF THE FIGURES AND TABLES

The following figures and tables are meant to be illustrative without limiting the scope and content of the instant disclosure or the claims herein.

FIG. 1A-1D provides a summary of the sequence identifies used in the present disclosure of various enzymes and sequence motifs.

FIGS. 2A-2B: FIG. 2A provides conserved residues of T. reesei Eg4, inferred from sequence alignment and the known structures of TrEGb (or T. reesei Eg7, also termed “TrEG7”) (crystal structure at Protein Data Bank Accession: pdb:2vtc) and TtEG (crystal structure at Protein Data Bank Accession: pdb:3E11). FIG. 2B provides conserved CBM domain residues inferred from sequence alignment with known sequences of Tr6A, Tr7A.

FIG. 3: provides conserved active site residues among Fv3C homologs, predicted based on the crystal structure of T. neapolitana Bgl3B complexed with glucose in −1 subsite (crystal structure at Protein Data Bank Accession: pdb:2X41).

FIG. 4: provides the enzyme composition of a fermentation broth produced by the T. reesei integrated strain H3A. The determination of this composition is described in Example 2.

FIG. 5: lists the enzymes (purified or unpurified) that were individually added to each of the samples in Example 2, and the stock protein concentrations of these enzymes.

FIG. 6: provides a T. reesei Eg4 dosing chart for Example 4 (experiment 1). The sample “#27” is an H3A/Eg4 integrated strain as described in Example 4. The amounts of purified T. reesei Eg4 that were added were listed under “Sample Description” either by wt. % or by mass (in mg protein/g G+X).

FIGS. 7A-7B: FIG. 7A provides another T. reesei Eg4 dosing chart for Example 4 (experiment 2). The samples are described similarly to those in FIG. 6. The amounts of purified T. reesei Eg4 that were added varied by smaller increments than those of Example 4, experiment 1 (above); FIG. 7B provides another T. reesei Eg4 dosing chart for Example 4 (experiment 3). The samples are described similarly to those in FIGS. 6 and 7A. The amounts of purified T. reesei Eg4 that were added varied by even finer increments than those of Example 4, experiments 1 and 2 (above).

FIGS. 8A-8B: FIG. 8A depicts the various ratios of CBH1, CBH2 and T. reesei Eg2 mixtures, as described in Example 15. FIGS. 8B-1 and 8B-2 lists glucan conversion (%) using various enzyme compositions. The experimental conditions are described in Example 15.

FIG. 9: lists the %yield of xylose released from diluted ammonia pretreated corncob using an enzyme composition comprising T. reesei Eg4, according to Example 6.

FIG. 10: provides %yield of glucose released from diluted ammonia pretreated corncob using an enzyme composition comprising T. reesei Eg4, according to Example 6.

FIG. 11: provides %yield of total fermentable monomers released from diluted ammonia pretreated corncob using an enzyme composition comprising T. reesei Eg4, according to Example 6.

FIG. 12: compares the amounts of glucose released through hydrolysis by an enzyme composition without T. reesei Eg4 vs. one with T. reesei Eg4 at 0.53 mg/g. The experiment is described in Example 7.

FIG. 13: lists β-glucosidase activity of a number of β-glucosidase homologs, including T. reesei Bgl1 (Tr3A), A. niger Bglu (An3A), Fv3C, Fv3D, and Pa3C. Activity on both cellobiose and CNPG substrates were measured, in accordance with Example 18.

FIG. 14: lists the relative weights of the enzymes in an enzyme mixture/composition tested in Example 19.

FIG. 15: provides a comparison of the effects of enzyme compositions on dilute ammonia pre-treated corncob. The experimental details are described in Example 21.

FIGS. 16A-16B: FIG. 16A depicts Fv3A nucleotide sequence (SEQ ID NO:1). FIG. 16B depicts Fv3A amino acid sequence (SEQ ID NO:2). The predicted signal sequence is underlined. The predicted conserved domain is in boldface type.

FIGS. 17A-17B: FIG. 17A depicts Pf43A nucleotide sequence (SEQ ID NO:3). FIG. 17B depicts Pf43A amino acid sequence (SEQ ID NO:4). The predicted signal sequence is underlined. The predicted conserved domain is in boldface type, the predicted carbohydrate binding module (“CBM”) is in uppercase type, and the predicted linker separating the CD and CBM is in italics.

FIGS. 18A-18B: FIG. 18A depicts Fv43E nucleotide sequence (SEQ ID NO:5). FIG. 18B depicts Fv43E amino acid sequence (SEQ ID NO:6). The predicted signal sequence is underlined. The predicted conserved domain is in boldface type.

FIGS. 19A-19B: FIG. 19A depicts Fv39A nucleotide sequence (SEQ ID NO:7). FIG. 19B depicts Fv39A amino acid sequence (SEQ ID NO:8). The predicted signal sequence is underlined. The predicted conserved domain is in boldface type.

FIGS. 20A-20B: FIG. 20A depicts Fv43A nucleotide sequence (SEQ ID NO:9). FIG. 20B depicts Fv43A amino acid sequence (SEQ ID NO:10). The predicted signal sequence is underlined. The predicted conserved domain is in boldface type, the predicted CBM is in uppercase type, and the predicted linker separating the conserved domain and CBM is in italics.

FIGS. 21A-21B: FIG. 21A depicts Fv43B nucleotide sequence (SEQ ID NO:11). FIG. 21B depicts Fv43B amino acid sequence (SEQ ID NO:12). The predicted signal sequence is underlined. The predicted conserved domain is in boldface type.

FIGS. 22A-22B: FIG. 22A depicts Pa51A nucleotide sequence (SEQ ID NO:13). FIG. 22B depicts Pa51A amino acid sequence (SEQ ID NO:14). The predicted signal sequence is underlined. The predicted L-α-arabinofuranosidase conserved domain is in boldface type. For expression in T. reesei, the genomic DNA was codon optimized for expression in T. reesei (see FIG. 39B).

FIGS. 23A-23B: FIG. 23A depicts Gz43A nucleotide sequence (SEQ ID NO:15). FIG. 23B depicts Gz43A amino acid sequence (SEQ ID NO:16). The predicted signal sequence is underlined. The predicted conserved domain is in boldface type. For expression in T. reesei, the predicted signal sequence was replaced by the T. reesei CBH1 signal sequence (myrklavisaflatara (SEQ ID NO: 117)).

FIGS. 24A-24B: FIG. 24A depicts Fo43A nucleotide sequence (SEQ ID NO:17). FIG. 24B depicts Fo43A amino acid sequence (SEQ ID NO:18). The predicted signal sequence is underlined. The predicted conserved domain is in boldface type. For expression in T. reesei, the predicted signal sequence was replaced by the T. reesei CBH1 signal sequence (myrklavisaflatara (SEQ ID NO:117)).

FIGS. 25A-25B: FIG. 25A depicts Af43A nucleotide sequence (SEQ ID NO:19). FIG. 25B depicts Af43A amino acid sequence (SEQ ID NO:20). The predicted conserved domain is in boldface type.

FIGS. 26A-26B: FIG. 26A depicts Pf51A nucleotide sequence (SEQ ID NO:21). FIG. 26B depicts Pf51A amino acid sequence (SEQ ID NO:22). The predicted signal sequence is underlined. The predicted L-α-arabinofuranosidase conserved domain is in boldface type. For expression in T. reesei, the predicted signal sequence was replaced by the T. reesei CBH1 signal sequence (myrklavisaflatara (SEQ ID NO:117)) and the Pf51A nucleotide sequence was codon optimized for expression in T. reesei

FIGS. 27A-27B: FIG. 27A depicts AfuXyn2 nucleotide sequence (SEQ ID NO:23). FIG. 27B depicts AfuXyn2 amino acid sequence (SEQ ID NO:24). The predicted signal sequence is underlined. The predicted GH11 conserved domain is in boldface type.

FIGS. 28A-28B: FIG. 28A depicts AfuXyn5 nucleotide sequence (SEQ ID NO:25). FIG. 28B depicts AfuXyn5 amino acid sequence (SEQ ID NO:26). The predicted signal sequence is underlined. The predicted GH11 conserved domain is in boldface type.

FIGS. 29A-29B: FIG. 29A depicts Fv43D nucleotide sequence (SEQ ID NO:27). FIG. 29B depicts Fv43D amino acid sequence (SEQ ID NO:28). The predicted signal sequence is underlined. The predicted conserved domain is in boldface type.

FIGS. 30A-30B: FIG. 30A depicts Pf43B nucleotide sequence (SEQ ID NO:29). FIG. 30B depicts Pf43B amino acid sequence (SEQ ID NO:30). The predicted signal sequence is underlined. The predicted conserved domain is in boldface type.

FIGS. 31A-31B: FIG. 31A depicts Fv51A nucleotide sequence (SEQ ID NO:31). FIG. 31B depicts Fv51A amino acid sequence (SEQ ID NO:32). The predicted signal sequence is underlined. The predicted L-α-arabinofuranosidase conserved domain is in boldface type.

FIGS. 32A-32B: FIG. 32A depicts Cg51B nucleotide sequence (SEQ ID NO:33). FIG. 32B depicts Cg51B amino acid sequence (SEQ ID NO:34). The predicted signal sequence is underlined. The predicted conserved domain is in boldface type.

FIGS. 33A-33B: FIG. 33A depicts Fv43C nucleotide sequence (SEQ ID NO:35). FIG. 33B depicts Fv43C amino acid sequence (SEQ ID NO:36). The predicted signal sequence is underlined. The predicted conserved domain is in boldface type.

FIGS. 34A-34B: FIG. 34A depicts Fv30A nucleotide sequence (SEQ ID NO:37). FIG. 34B depicts Fv30A amino acid sequence (SEQ ID NO:38). The predicted signal sequence is underlined.

FIGS. 35A-35B: FIG. 35A depicts Fv43F nucleotide sequence (SEQ ID NO:39). FIG. 35B depicts Fv43F amino acid sequence (SEQ ID NO:40). The predicted signal sequence is underlined.

FIGS. 36A-36B: FIG. 36A depicts T. reesei Xyn3 nucleotide sequence (SEQ ID NO:41). FIG. 36B depicts T. reesei Xyn3 amino acid sequence (SEQ ID NO:42). The predicted signal sequence is underlined. The predicted conserved domain is in boldface type.

FIGS. 37A-37B: FIG. 37A depicts amino acid sequence of T. reesei Xyn2 (SEQ ID NO:43). The signal sequence is underlined. The predicted conserved domain is in bold face type. The coding sequence can be found in Törrönen et al. Biotechnology, 1992, 10:1461-65; FIG. 37B depicts amino acid sequence of Pa3C (SEQ ID NO:44), a GH3 enzyme from P. anserina.

FIG. 38 depicts amino acid sequence of T. reesei Bxl1 (SEQ ID NO:45). The signal sequence is underlined. The predicted conserved domain is in bold face type. The coding sequence can be found in Margolles-Clark et al. Appl. Environ. Microbiol. 1996, 62(10):3840-46.

FIGS. 39A-39E: FIG. 39A depicts deduced cDNA for Pa51A (SEQ ID NO:46). FIG. 39B depicts codon optimized cDNA for Pa51A (SEQ ID NO:47). FIG. 39C: Coding sequence for a construct comprising a CBH1 signal sequence (underlined) upstream of genomic DNA encoding mature Gz43A (SEQ ID NO:48). FIG. 39D: Coding sequence for a construct comprising a CBH1 signal sequence (underlined) upstream of genomic DNA encoding mature Fo43A (SEQ ID NO:49). FIG. 39E: Coding sequence for a construct comprising a CBH1 signal sequence (underlined) upstream of codon optimized DNA encoding Pf51A (SEQ ID NO:50).

FIGS. 40A-40B: FIG. 40A depicts nucleotide sequence of T. reesei Eg4 (SEQ ID NO:51). FIG. 40B depicts amino acid sequence of T. reesei Eg4 (SEQ ID NO:52). The predicted signal sequence is underlined. The predicted conserved domains are in bold type fonts. The predicted linker is in italic type fonts.

FIGS. 41A-41B: FIG. 41A depicts nucleotide sequence of Pa3D (SEQ ID NO:53). FIG. 41B depicts amino acid sequence of Pa3D (SEQ ID NO:54). The predicted signal sequence is underlined. The predicted conserved domains are in bold type fonts.

FIGS. 42A-42B: FIG. 42A depicts nucleotide sequence of Fv3G (SEQ ID NO:55). FIG. 42B depicts amino acid sequence of Fv3G (SEQ ID NO:56). The predicted signal sequence is underlined. The predicted conserved domains are in bold type fonts.

FIGS. 43A-43B: FIG. 43A depicts nucleotide sequence of Fv3D (SEQ ID NO:57). FIG. 43B depicts amino acid sequence of Fv3D (SEQ ID NO:58). The predicted signal sequence is underlined. The predicted conserved domains are in bold type fonts.

FIGS. 44A-44B: FIG. 44A depicts nucleotide sequence of Fv3C (SEQ ID NO:59). FIG. 44B depicts amino acid sequence of Fv3C (SEQ ID NO:60). The predicted signal sequence is underlined. The predicted conserved domains are in bold type fonts.

FIGS. 45A-45B: FIG. 45A depicts nucleotide sequence of Tr3A (SEQ ID NO:61). FIG. 45B depicts amino acid sequence of Tr3A (SEQ ID NO:62). The predicted signal sequence is underlined. The predicted conserved domains are in bold type fonts.

FIGS. 46A-46B: FIG. 46A depicts nucleotide sequence of Tr3B (SEQ ID NO:63). FIG. 46B depicts amino acid sequence of Tr3B (SEQ ID NO:64). The predicted signal sequence is underlined. The predicted conserved domains are in bold type fonts.

FIGS. 47A-47B: FIG. 47A depicts the codon-optimized (for expression in T. reesei) nucleotide sequence of Te3A (SEQ ID NO:65). FIG. 47B depicts amino acid sequence of Te3A (SEQ ID NO:66). The predicted signal sequence is underlined. The predicted conserved domains are in bold type fonts.

FIGS. 48A-48B: FIG. 48A depicts nucleotide sequence of An3A (SEQ ID NO:67). FIG. 48B depicts amino acid sequence of An3A (SEQ ID NO:68). The predicted signal sequence is underlined. The predicted conserved domains are in bold type fonts.

FIGS. 49A-49B: FIG. 49A depicts nucleotide sequence of Fo3A (SEQ ID NO:69). FIG. 49B depicts amino acid sequence of Fo3A (SEQ ID NO:70). The predicted signal sequence is underlined. The predicted conserved domains are in bold type fonts.

FIGS. 50A-50B: FIG. 50A depicts nucleotide sequence of Gz3A (SEQ ID NO:71). FIG. 50B depicts amino acid sequence of Gz3A(SEQ ID NO:72). The predicted signal sequence is underlined. The predicted conserved domains are in bold type fonts.

FIGS. 51A-51B: FIG. 51A depicts nucleotide sequence of Nh3A (SEQ ID NO:73). FIG. 51B depicts amino acid sequence of Nh3A (SEQ ID NO:74). The predicted signal sequence is underlined. The predicted conserved domains are in bold type fonts.

FIGS. 52A-52B: FIG. 52A depicts nucleotide sequence of Vd3A (SEQ ID NO:75). FIG. 52B depicts amino acid sequence of Vd3A (SEQ ID NO:76). The predicted signal sequence is underlined. The predicted conserved domains are in bold type fonts.

FIGS. 53A-53B: FIG. 53A depicts nucleotide sequence of Pa3G(SEQ ID NO:77). FIG. 53B depicts amino acid sequence of Pa3G (SEQ ID NO:78). The predicted signal sequence is underlined. The predicted conserved domains are in bold type fonts.

FIG. 54: depicts amino acid sequence of Tn3B (SEQ ID NO:79). The standard signal prediction program, Signal P provided no predicted signal sequence.

FIG. 55A-1 to FIG. 55A-7: depicts an amino acid sequence alignment of certain β-glucosidase homologs.

FIGS. 56A-56B: depicts an amino acid sequence alignment of T. reesei Eg4 with TrEGb (or TrEG7 (SEQ ID NO:80) and TtEG (SEQ ID NO:81).

FIG. 57: depicts a partial amino acid sequence alignment of the CBM domains of T. reesei Eg4 with Tr6A (SEQ ID NO:82) and with Tr7A (SEQ ID NO:83), as well as two GH61/endoglucanases from T. aurantiacus (SEQ ID NOs:206 and 207).

FIG. 58A-58D: FIG. 58A depicts glucose release following saccharification of dilute ammonia pretreated corncob by adding enzyme compositions comprising various purified or non-purified enzymes of FIG. 5, which were added to T. reesei integrated strain H3A, in accordance with Example 2. FIG. 58B depicts cellobiose release following saccharification of dilute ammonia pretreated corncob by adding enzyme compositions comprising various purified or non-purified enzymes of FIG. 5, which were added to T. reesei integrated strain H3A, in accordance with Example 2; FIG. 58C depicts xylobiose release following saccharification of dilute ammonia pretreated corncob by adding enzyme compositions comprising various purified or non-purified enzymes of FIG. 5, which were added to T. reesei integrated strain H3A, in accordance with Example 2; FIG. 58D depicts xylose release following saccharification of dilute ammonia pretreated corncob by adding enzyme compositions comprising various purified or non-purified enzymes of FIG. 5, which were added to T. reesei integrated strain H3A, in accordance with Example 2.

FIGS. 59A-59B: FIG. 59A depicts the expression cassette pEG1-EG4-sucA, as described in Example 3; FIG. 59B depicts the plasmid map of pCR Blunt II TOPO containing expression cassette pEG1-EG4-sucA, as described in Example 3.

FIG. 60: depicts the amount/percentage of glucan/xylan conversion to cellobiose/glucose by an enzyme composition comprising enzymes produced by the T. reesei integrated strain H3A transformants expressing T. reesei Eg4, according to Example 3.

FIG. 61: depicts the increased percent glucan conversion observed using an increasing amount of an enzyme composition produced by H3A transformants expressing T. reesei Eg4. The experimental details are described in Example 3.

FIGS. 62A-62G: FIG. 62A depicts the plasmid map of pCR-Blunt II TOPO plasmid including the pEG1-Fv51A expression cassette, as described in Example 23; FIG. 62B depicts the plasmid map of pCR-Blunt II TOPO plasmid including pEG1-Fv3A with the cbh1 terminator sequence, as described in Example 23; FIG. 62C depicts the plasmid map of pCR-Blunt II TOPO plasmid including Pcbh2-Fv43D, as described in Example 23; FIG. 62D depicts the plasmid map of pCR-Blunt II-TOPO plasmid including Pcbh2-Fv43D-als marker (pSK49), as described in Example 23; FIG. 62E depicts the plasmid map of pCR-Blunt II-TOPO with Pcbh2-Fv43D (pSK42), as described in Example 23; FIG. 62F depicts the plasmid map of pTrex6g including Fv3A sequence, as described in Example 23; FIG. 62G depicts the plasmid map of pTrex6G with Fv43D sequence, as described in Example 23.

FIGS. 63A-63B: FIG. 63A depicts glucose production from corncob hydrolysis using various enzyme compositions, in accordance with the experiments described in Example 16; FIG. 63B depicts xylose production from corncob hydrolysis using various enzyme compositions in accordance with the description of Example 16.

FIG. 64 depicts the effect of T. reesei Eg4 on glucose release from saccharification of dilute ammonia pretreated corncob. The Y-axis refers to the concentrations of glucose or xylose released in the reaction mixtures. The X axis lists the names/brief descriptions of the enzyme composition samples. The experimental details are in Example 4.

FIG. 65 depicts the effect of T. reesei Eg4 on xylose release from saccharification of dilute ammonia pretreated corncob. The Y-axis refers to the concentrations of glucose or xylose released in the reaction mixtures. The X axis lists the names/brief descriptions of the enzyme composition samples. The experimental details are described in Example 4.

FIGS. 66A-66B: FIG. 66A depicts the effect of T. reesei Eg4 in various amounts (0.05 mg/g to 1.0 mg/g) on glucose release from saccharification of dilute ammonia pretreated corncob, as described in Example 4. FIG. 66B depicts the effect of T. reesei Eg4 in various amounts (0.1 mg/g to 0.5 mg/g) on glucose release from saccharification of dilute ammonia pretreated corncob, as described in Example 4.

FIG. 67: depicts the effect of T. reesei Eg4 in an enzyme composition on glucose and xylose release from saccharification of dilute ammonia pretreated corn stover, at various solids lodings, as described in Example 5.

FIG. 68: depicts the glucose monomer release as a result of treating ammonia pretreated corncob using purified T. reesei Eg4 alone, in accordance with Example 7.

FIG. 69: depicts and compares the saccharification performance on various substrates of the enzyme compositions produced by the T. reesei integrated strain H3A and the integrated strain H3A/Eg4 (strain #27), at an enzyme dosage of 14 mg/g, according to Example 8.

FIG. 70: depicts the saccharification performance of the enzyme compositions produced by the T. reesei integrated strain H3A and the integrated strain H3A/Eg4 (strain #27), at various enzyme dosages, on acid pretreated corn stover according to Example 9.

FIG. 71: depicts the saccharification performance of the enzyme compositions produced by the T. reesei integrated strain H3A and the integrated strain H3A/Eg4 (strain #27) on dilute ammonia pretreated corn leaves, stalks, or cobs, according to Example 10.

FIGS. 72A-1 to 72A-3 (left panel)-72B-1 to 72B-3 (right panel): FIG. 72A-1 to 72A-3 depicts amounts for various enzyme compositions for saccharification; FIG. 72B-1 to 72B-3 depicts the amount of glucose, glucose+cellobiose, or xylose produced with each enzyme composition corresponding to FIG. 72A-1 to 72A-3. Experimental details are found in Example 14.

FIG. 73: compares saccharification performance, in terms of the amounts of glucose or xylose released, of enzyme compositions produced by the T. reesei integrated strain H3A and the integrated strain H3A/Eg4 (strain #27), in accordance with Example 11.

FIG. 74: depicts the change in percent glucan and xylan conversion at increasing amounts of an enzyme composition produced by the T. reesei integrated strain H3A/Eg4 (strain #27), in accordance with Example 12.

FIG. 75: depicts the effect of T. reesei Eg4 addition on dilute ammonia pretreated corncob saccharification, in accordance with Example 13 part A.

FIG. 76: depicts CMC hydrolysis by T. reesei Eg4, according to Example 13 part B.

FIG. 77: depicts cellobiose hydrolysis by T. reesei Eg4, according to Example 13 part C.

FIG. 78: depicts a pENTR/D-TOPO vector with the Fv3C open reading frame, as described in Example 17.

FIGS. 79A-79B: FIG. 79A depicts an expression vector pTrex6g, as in Example 17; FIG. 79B depicts a pExpression construct pTrex6g/Fv3C, as in Example 17.

FIG. 80 depicts predicted coding region of Fv3C genomic DNA sequence, as described in Example 17.

FIGS. 81A-81B: FIG. 81A depicts N-terminal amino acid sequence of Fv3C. The arrows show the putative signal peptide cleavage sites. The start of the mature protein is underlined. FIG. 81B depicts an SDS-PAGE gel of T. reesei transformants expressing Fv3C from the annotated (1) and alternative (2) start codons, in accordance with Example 17.

FIG. 82: compares performance of whole cellulase plus β-glucosidase mixtures in saccharification of phosphoric acid swollen cellulose at 50° C. Whole cellulase at 10 mg protein/g cellulose was blended with 5 mg/g β-glucosidase and the enzyme mixtures used to hydrolyze phosphoric acid swollen cellulose at 0.7% cellulose, pH 5.0. The sample labeled as background in the figure was the conversion obtained from 10 mg/g whole cellulase alone without added β-glucosidase. Reactions were carried out in microtiter plates at 50° C. for 2 h. The samples were tested in triplicates, according to Example 19, part A.

FIG. 83: compares performance of whole cellulase plus β-glucosidase mixtures in saccharification of acid pre-treated cornstover (PCS) at 50° C. Whole cellulase at 10 mg protein/g cellulose was blended with 5 mg/g β-glucosidase and the enzyme mixtures used to hydrolyze PCS at 13% solids, pH 5.0. The sample labeled as background was the conversion obtained from 10 mg/g whole cellulase alone without added β-glucosidase. Reactions were carried out in microtiter plates at 50° C. for 48 h. The samples were tested in triplicates, in accordance with Example 19, part B.

FIG. 84: compares performance of whole cellulase plus β-glucosidase mixtures in saccharification of ammonia pretreated corncob at 50° C. Whole cellulase at 10 mg protein/g cellulose was blended with 8 mg/g hemicellulases and 5 mg/g β-glucosidase and the enzyme mixtures used to hydrolyze the ammonia pretreated corncob at 20% solids, pH 5.0. The sample labeled as background was the conversion obtained from 10 mg/g whole cellulase+8 mg/g hemicellulose mix alone without added β-glucosidase. Reactions were carried out in microtiter plates at 50° C. for 48 h. The samples were assayed in triplicates, in accordance with Example 19, part C.

FIG. 85: compares performance of whole cellulase plus β-glucosidase mixtures in saccharification of sodium hydroxide (NaOH) pretreated corncob at 50° C. Whole cellulase at 10 mg protein/g cellulose was blended with 5 mg/g β-glucosidase and the enzyme mixtures used to hydrolyze the NaOH pretreated corncob at 17% solids, pH 5.0. The sample labeled as background was the conversion obtained from 10 mg/g whole cellulase mix alone without added β-glucosidase. Reactions were carried out in microtiter plates at 50° C. for 48 h. Each sample was assayed in 4 replicates, according to Example 19, part D.

FIG. 86: compares performance of whole cellulase plus β-glucosidase mixtures in saccharification of dilute ammonia pretreated switchgrass at 50° C. Whole cellulase at 10 mg protein/g cellulose was blended with 5 mg/g β-glucosidase and the enzyme mixtures used to hydrolyze switchgrass at 17% solids, pH 5.0. The sample labeled as background was the conversion obtained from 10 mg/g whole cellulase mix alone without added β-glucosidase. Reactions were carried out in microtiter plates at 50° C. for 48 h. Each sample was assayed in 4 replicates, in accordance with Example 19, part E.

FIG. 87: compares performance of whole cellulase plus β-glucosidase mixtures in saccharification of AFEX cornstover at 50° C. Whole cellulase at 10 mg protein/g cellulose was blended with 5 mg/g β-glucosidase and the enzyme mixtures used to hydrolyze AFEX cornstover at 14% solids, pH 5.0. The sample labeled as background was the conversion obtained from 10 mg/g whole cellulase mix alone without added β-glucosidase. Reactions were carried out in microtiter plates at 50° C. for 48 h. Each sample was assayed in 4 replicates, in accordance with Example 19, part F.

FIGS. 88A-88C: depict percent glucan conversion from dilute ammonia pretreated corncob at 20% solids at varying ratios of β-glucosidase to whole cellulase, in an amount of between 0 and 50%. The enzyme dosage was kept constant for each of the experiments. FIG. 88A depicts the experiment conducted with T. reesei Bgl1. FIG. 88B depicts the experiment conducted with Fv3C. FIG. 88C depicts the experiment conducted with A. niger Bglu (An3A). Experimental details are found in Example 20 herein.

FIG. 89: depicts percent glucan conversion from dilute ammonia pretreated corncob at 20% solids by three different enzyme compositions dosed at levels of 2.5-40 mg/g glucan, in accordance with Example 21. Δ marks glucan conversion observed with Accellerase 1500+Multifect Xylanase, ⋄ marks glucan conversion observed with a whole cellulase from T. reesei integrated strain H3A, ♦ marks glucan conversion observed with an enzyme composition comprising 75 wt. % whole cellulase from T. reesei integrated strain H3A plus 25 wt. % Fv3C.

FIGS. 90A-90I: FIG. 90A depicts a map of pRAX2-Fv3C expression plasmid used for expression in A. niger, as described in Example 22. FIG. 90B depicts pENTR-TOPO-Bgl1-943/942 plasmid, as described in Example 2. FIG. 90C depicts pTrex3g 943/942 vector, as described in Example 2. FIG. 90D depicts pENTR/T. reesei Xyn3 plasmid, as described in Example 2. FIG. 90E depicts pTrex3g/T. reesei Xyn3 expression vector, as described in Example 2. FIG. 90F depicts pENTR-Fv3A plasmid, as described in Example 2. FIG. 90G depicts pTrex6g/Fv3A expression vector, as described in Example 2. FIG. 90H depicts TOPO Blunt/Pegl1-Fv43D plasmid, as described in Example 2. FIG. 90I depicts TOPO Blunt/Pegl1-Fv51A plasmid, as described in Example 2.

FIG. 91: depicts an amino acid alignment between T. reesei β-xylosidase and Fv3A.

FIG. 92: depicts an amino acid sequence alignment of certain GH39 β-xylosidases. Underlined residues in bold face are the predicted catalytic general acid-base residue (marked with “A” above the alignment) and catalytic nucleophile residue (marked with “N” above the alignment). Underlined residues in normal face in the bottom two sequences are within 4 Å of the substrate in the active sites of the respective 3D structures (pdb: 1uhv and 2bs9, respectively). Underlined residues in the Fv39A sequence are predicted to be within 4 Å of a bound substrate in the active site.

FIG. 93A-93B: depicts an amino acid sequence alignment of certain GH43 family hydrolases. Amino acid residues conserved among members of the family are underlined and in bold face.

FIG. 94: depicts an amino acid sequence alignment of certain GH51 family enzymes. Amino acid residues conserved among members of the family are shown underlined and in bold face.

FIG. 95A-95B: depict amino acid sequence alignments of certain GH10 and GH11 family endoxylanases. FIG. 95A: Alignment of GH10 family xylanases. Underlined residues in bold face are the the catalytic nucleophile residues (marked with “N” above the alignment). FIG. 95B: Alignment of GH11 family xylanases. Underlined residues in bold face are the the catalytic nucleophile residues and general acid base residues (marked with “N” and “A”, respectively, above the alignment).

FIG. 96A-96B: depicts an amino acid sequence alignment of a number of GH3 family hydrolases. Amino acid residues highly conserved among members of the family are shown underlined and in bold face type.

FIG. 97: depicts an amino acid sequence alignment of two representative Fusarium GH30 family hydrolases. Amino acid residues that are conserved among members of the family are shown underlined and in bold face type.

FIG. 98 lists a number of amino acid sequence motifs of GH61 endoglucanases.

FIGS. 99A-99C: FIG. 99A depicts a schematic representation of the gene encoding the Fv3C/T. reesei Bgl3 chimeric/fusion polypeptide. FIG. 99B-1 to 99B-2 depicts the nucleotide sequence encoding the fusion/chimeric polypeptide Fv3C/T. reesei Bgl3 (SEQ ID NO:92). FIG. 99C depicts the amino acid sequence encoding the fusion/chimeric polypeptide Fv3C/T. reesei Bgl3 (SEQ ID NO:93). The sequence in bold type is from T. reesei Bgl3. Experimental details are described in Example 23.

FIG. 100: is a map of pTTT-pyrG13-Fv3C/Bgl3 fusion plasmid as in Example 23.

FIGS. 101A-101B: FIG. 101A depicts the nucleotide sequence encoding the Fv3C/Te3A/T. reesei Bgl3 chimera (SEQ ID NO:92); FIG. 101B depicts the amino acid sequence encoding the Fv3C/Te3A/T. reesei Bgl3 chimera (SEQ ID NO:95)

FIGS. 102A-102B: FIG. 102A: is a table listing suitable amino acid sequence motifs of a β-glucosidase polypeptide, including, e.g., variants, mutants, or fusion/chimeric polypeptides thereof. FIG. 102B: is a table listing the amino acid sequence motifs used to design a β-glucosidase polypeptide hybrid/chimera.

FIGS. 103A-103C: FIG. 103A depicts a pTTT-pyrG13-FAB (i.e., Fv3C/Te3A/Bgl3 chimera) fusion plasmid; FIG. 103B depicts a pCR-Blunt II-Pcbh2-xyn3-cbh1 terminator plasmid; FIG. 103C depicts a pCR-Blunt II-TOPO/Pegl1-Egl4-suc plasmid. Experimental details are found in Example 23.

FIG. 104 depicts and compares the saccharification performance of transformants on dilute ammonia pretreated corncob. Strains with good xylan and glucan conversions were selected for further characterization, according to Example 23.

FIGS. 105A-J: FIG. 105A depicts 3-D superimposed structures of Fv3C and Te3A, and T. reesei Bgl1, viewed from a first angle, rendering visible the structure of “insertion 1.” FIG. 105B depicts the same superimposed structures viewed from a second angle, rendering visible the structure of “insertion 2.” FIG. 105C depicts the same superimposed structures viewed from a third angle, rendering visible the structure of “insertion 3.” FIG. 105D depicts the same superimposed structures, viewed from a fourth angle, rendering visible the structure of “insertion 4.” FIG. 105E-1 to 105E-2_is a sequence alignment of T. reesei Bgl1 (Q12715_TRI), Te3A (ABG2_T_eme), and Fv3C (FV3C), marked with insertions 1-4, which are all loop-like structures. FIG. 105F depicts superimposed parts of structures of Fv3C (light grey), Te3A (dark grey), and T. reesei Bgl1 (black), indicating conserved interactions of between residues W59/W33 and W355/W325 (Fv3C/Te3A). FIG. 105G depicts superimposed parts of of structures of Fv3C (light grey), Te3A (dark grey), and T. reesei Bgl1 (black), indicating conserved interactions between the first pair of residues: S57/31 and N291/261 (Fv3C/Te3A); and between the second group of residues: Y55/29, P775/729 and A778/732 (Fv3C/Te3A). FIG. 105H depicts superimposed parts of structures Fv3C (dark grey), and T. reesei Bgl1 (black), indicating hydrogen bonding Interactions of Fv3C at K162 with the backbone oxygen atom of V409 in “insertion 2,” an interaction that is conserved in Te3A, but not found in T. reesei Bgl1. FIG. 105I(a)-(b) depict conserved glycosylation sites within SEQ ID NO: 201, shared amongst Fv3C, Te3A and a chimeric/hybrid β-glucosidase of SEQ ID NO: 95, (a) depicts the same region superimposed with Te3A (dark grey) and T. reesei Bgl1(black); (b) depicts the same region superimposed with the chimeric/hybrid β-glucosidase of SEQ ID NO: 95 (light grey), Te3A (dark grey) and T. reesei Bgl1(black). The black arrow indicates the loop structure of “insertion 3” in Te3A (also present in the hybrid β-glucosidase of SEQ ID NO: 95), which appeared to bury the glycosylation glycans. FIG. 105J depicts superimposed parts of of structures of Fv3C (light grey), Te3A (dark grey), and T. reesei Bgl1 (black), indicating conserved interactions between residues W386/355 interacts with W95/68 (Fv3C/Te3A) of “insertion 2” of Fv3C and Te3A. The interaction is missing from T. reesei Bgl1.

FIGS. 106A-B: FIG. 106A: depicts a representative UPLC trace of an enzyme composition as described in Example 24. FIG. 106B: is a table listing the measured amounts of enzyme components of the enzyme composition in the same Example.

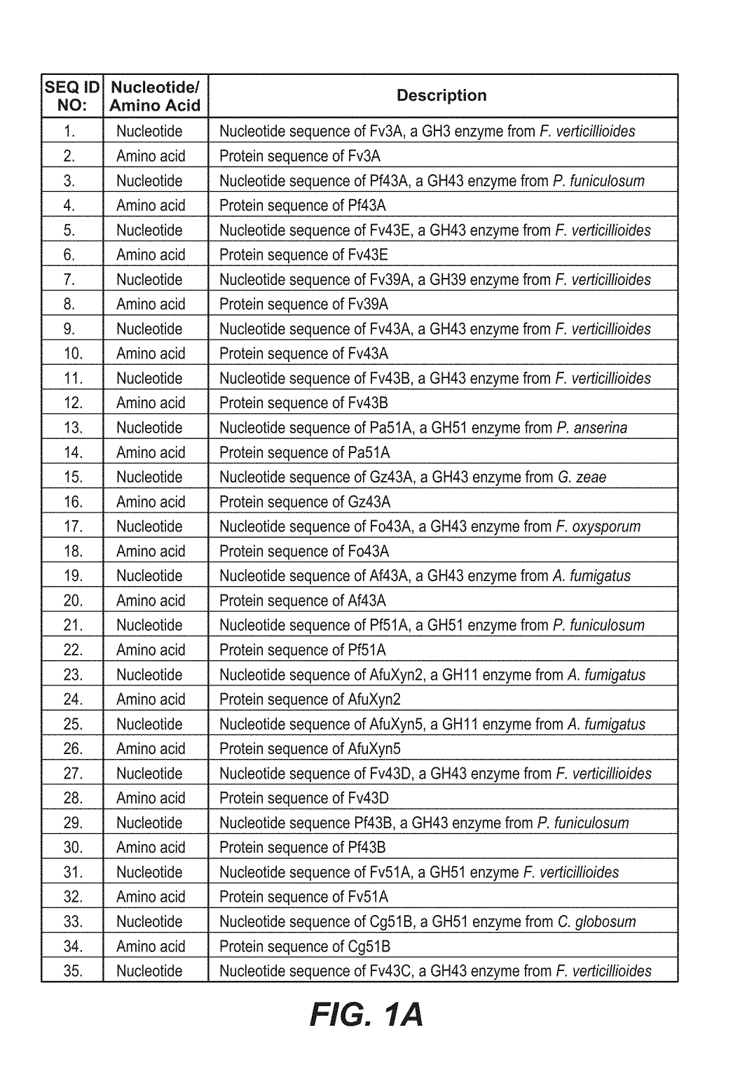

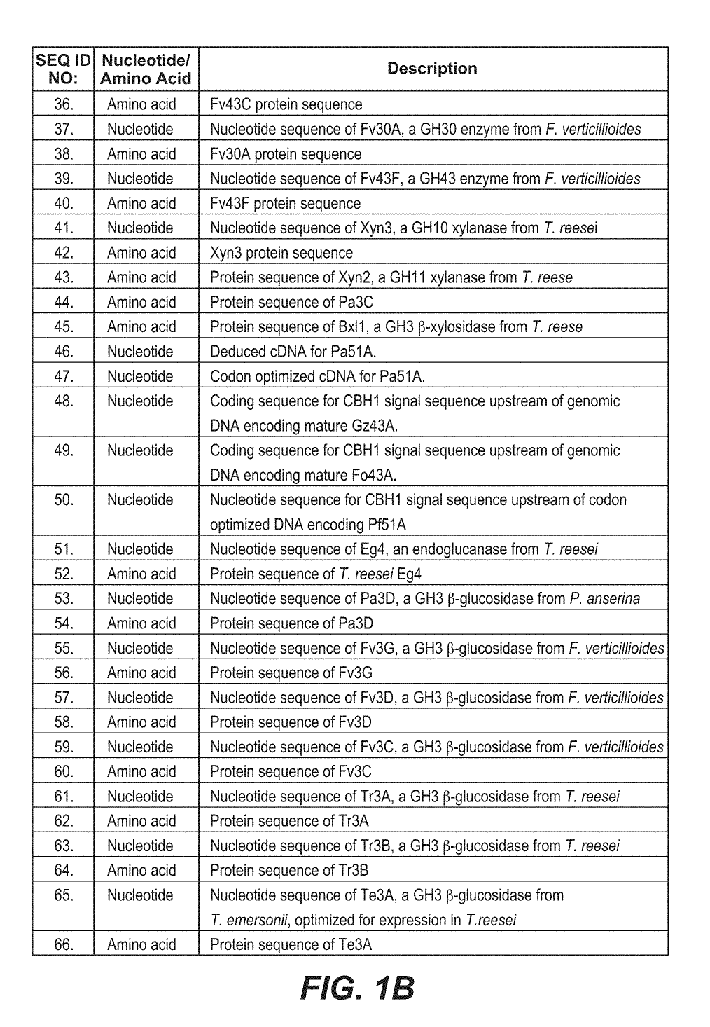

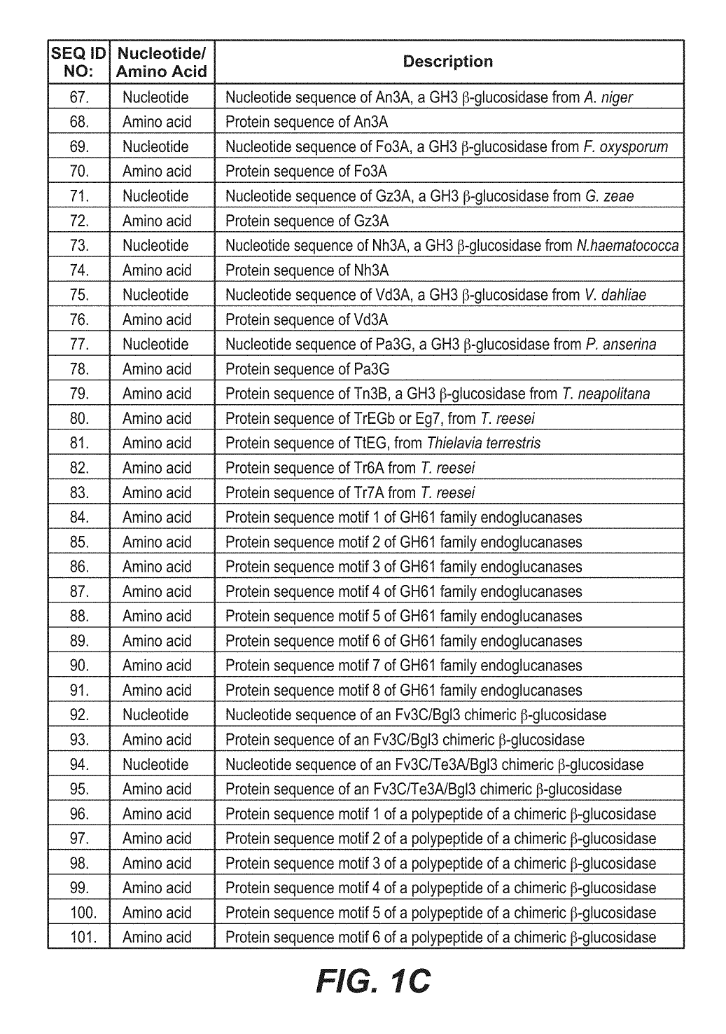

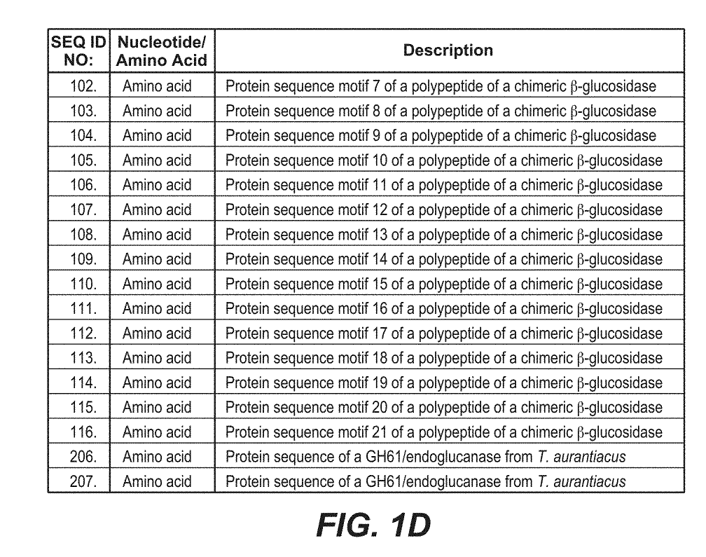

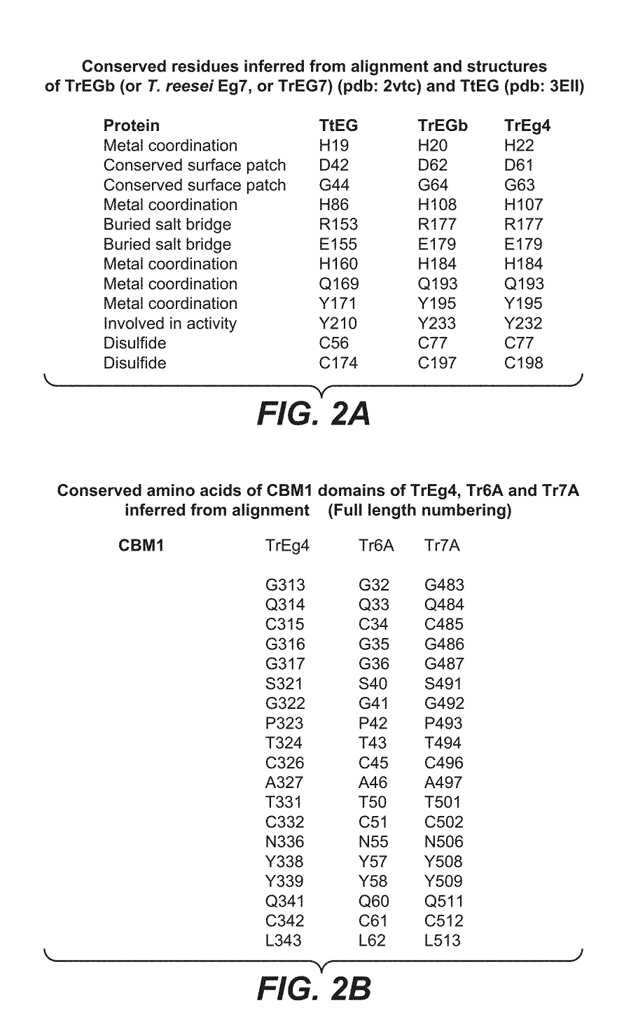

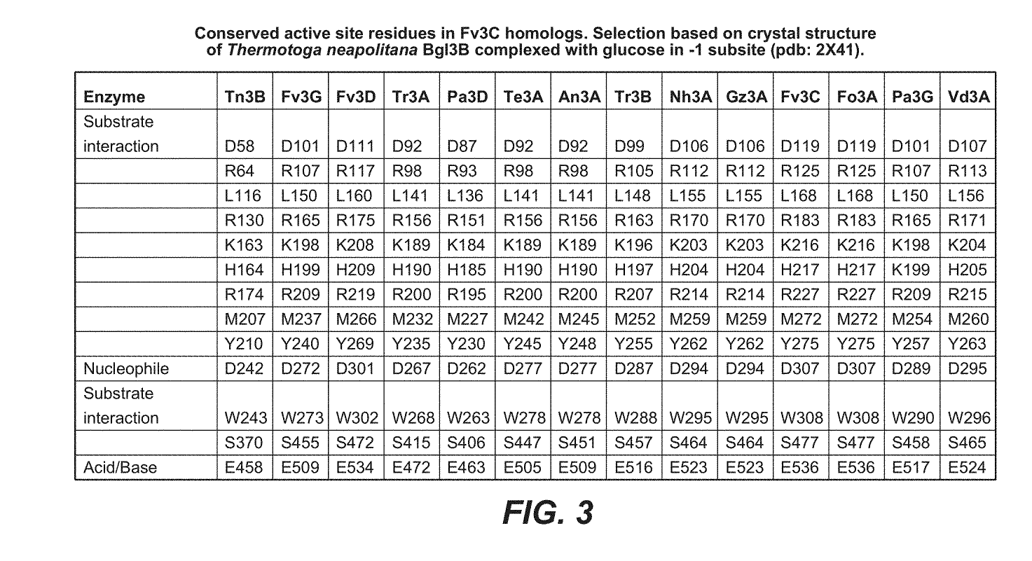

### 5. DETAILED DESCRIPTION

Enzymes have traditionally been classified by substrate specificity and reaction products. In the pre-genomic era, function was regarded as the most amenable (and perhaps most useful) basis for comparing enzymes and assays for various enzymatic activities have been well-developed for many years, resulting in the familiar EC classification scheme. Cellulases and other glycosyl hydrolases, which act upon glycosidic bonds between carbohydrate moieties (or a carbohydrate and non-carbohydrate moiety-as occurs in nitrophenol-glycoside derivatives) are, under this classification scheme, designated as EC 3.2.1.-, with the final number indicating the exact type of bond cleaved. For example, an endo-acting cellulase (1,4β-endoglucanase) is designated EC 3.2.1.4. With the advent of widespread genome sequencing projects, sequencing data have facilitated analyses and comparison of related genes and proteins. Additionally, a growing number of enzymes capable of acting on carbohydrate moieties (i.e., carbohydrases) have been crystallized and their 3-D structures solved. Such analyses have identified discreet families of enzymes with related sequence, which contain conserved three-dimensional folds that can be predicted based on their amino acid sequence. Further, it has been shown that enzymes with the same or similar three-dimensional folds exhibit the same or similar stereo specificity of hydrolysis, even when catalyzing different reactions (Henrissat et al., FEBS Lett 1998, 425(2): 352-4; Coutinho and Henrissat, Genetics, biochemistry and ecology of cellulose degradation, 1999, T. Kimura. Tokyo, Uni Publishers Co: 15-23.). These findings form the basis of a sequence-based classification of carbohydrase modules, available in the form of an internet database, the Carbohydrate-Active enZYme server (CAZy), available at afmb.cnrs-mrs.fr/CAZY/index.html (Carbohydrate-active enzymes: an integrated database approach. See Cantarel et al., 2009, Nucleic Acids Res. 37 (Database issue):D233-38).

CAZy defines four major classes of carbohydrases distinguishable by the type of reaction catalyzed: Glycosyl Hydrolases (GH's), Glycosyltransferases (GT's), Polysaccharide Lyases (PL's), and Carbohydrate Esterases (CE's). The enzymes of the disclosure are glycosyl hydrolases. GH's are a group of enzymes that hydrolyze the glycosidic bond between two carbohydrates, or between a carbohydrate and a non-carbohydrate moiety. A classification system for glycosyl hydrolases, grouped by sequence similarity, has led to the definition of over 85 different families. This classification is available on the CAZy web site.

The enzymes of the disclosure belong, inter alia, to the glycosyl hydrolase families 3, 10, 11, 30, 39, 43, 51, and/or 61.

Glycoside hydrolase family 3 (“GH3”) enzymes include, e.g., β-glucosidase (EC:3.2.1.21); β-xylosidase (EC:3.2.1.37); N-acetyl β-glucosaminidase (EC:3.2.1.52); glucan β-1,3-glucosidase (EC:3.2.1.58); cellodextrinase (EC:3.2.1.74); exo-1,3-1,4-glucanase (EC:3.2.1); and β-galactosidase (EC 3.2.1.23). For example, GH3 enzymes can be those that have β-glucosidase, β-xylosidase, N-acetyl β-glucosaminidase, glucan β-1,3-glucosidase, cellodextrinase, exo-1,3-1,4-glucanase, and/or β-galactosidase activity. Generally, GH3 enzymes are globular proteins and can consist of two or more subdomains. A catalytic residue has been identified as an aspartate residue that, in β-glucosidases, located in the N-terminal third of the peptide and sits within the amino acid fragment SDW (Li et al. 2001, Biochem. J. 355:835-840). The corresponding sequence in Bgl1 from T. reesei is T266D267W268 (counting from the methionine at the starting position), with the catalytic residue aspartate being the D267. The hydroxyl/aspartate sequence is also conserved in the GH3 β-xylosidases tested. For example, the corresponding sequence in T. reesei Bxl1 is S310D311 and the corresponding sequence in Fv3A is S290D291.

Glycoside hydrolase family 39 (“GH39”) enzymes have α-L-iduronidase (EC:3.2.1.76) or β-xylosidase (EC:3.2.1.37) activity. The three-dimensional structure of two GH39 β-xylosidases, from T. saccharolyticum (Uniprot Accession No. P36906) and G.s stearothermophilus (Uniprot Accession No. Q9ZFM2), have been solved (see Yang et al. J. Mol. Biol. 2004, 335(1):155-65 and Czjzek et al., J. Mol. Biol. 2005, 353(4):838-46). The most highly conserved regions in these enzymes are located in their N-terminal sections, which have a classic (α/β)8 TIM barrel fold with the two key active site glutamic acids located at the C-terminal ends of β-strands 4 (acid/base) and 7 (nucleophile). Fv39A residues E168 and E272 are predicted to function as catalytic acid-base and nucleophile, respectively, based on a sequence alignment of the abovementioned GH39 β-xylosidases from T. saccharolyticum and G. stearothermophilus with Fv39A.

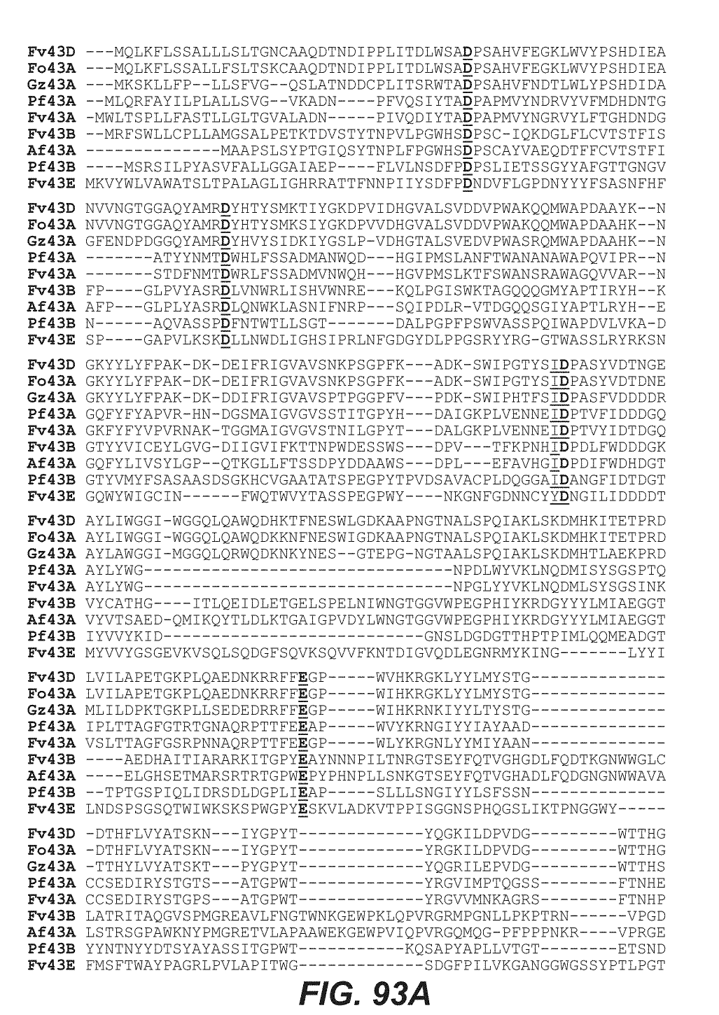

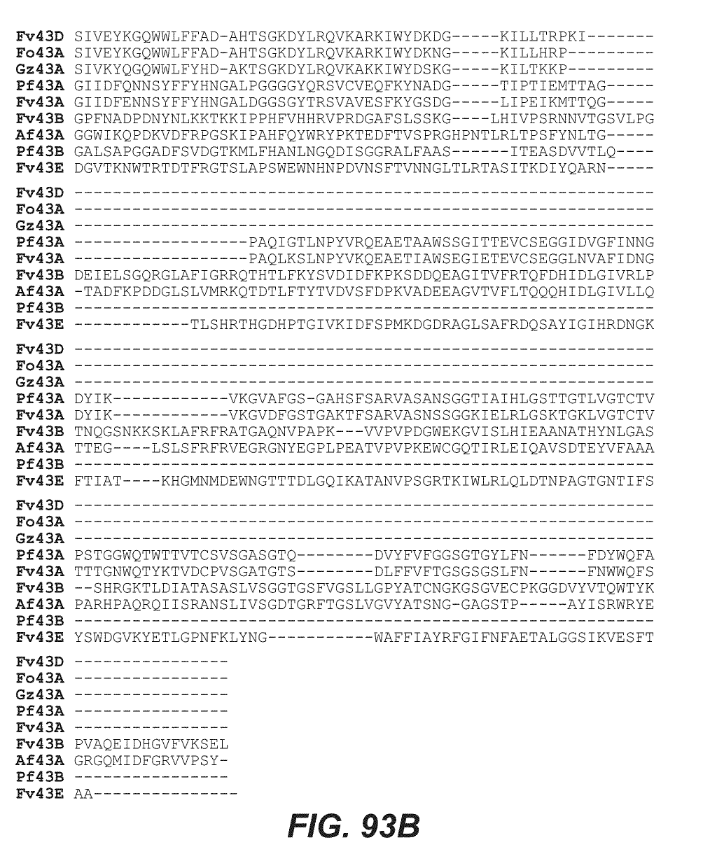

Glycoside hydrolase family 43 (“GH43”) enzymes include, e.g., L-α-arabinofuranosidase (EC 3.2.1.55); β-xylosidase (EC 3.2.1.37); endo-arabinanase (EC 3.2.1.99); and/or galactan 1,3-β-galactosidase (EC 3.2.1.145). For example, GH43 enzymes can have L-α-arabinofuranosidase activity, β-xylosidase activity, endo-arabinanase activity, and/or galactan 1,3-β-galactosidase activity. GH43 family enzymes display a five-bladed-β-propeller-like structure. The propeller-like structure is based upon a five-fold repeat of blades composed of four-stranded β-sheets. The catalytic general base, an aspartate, the catalytic general acid, a glutamate, and an aspartate that modulates the pKa of the general base were identified through the crystal structure of C. japonicus CjAbn43A, and confirmed by site-directed mutagenesis (see Nurizzo et al. Nat. Struct. Biol. 2002, 9(9) 665-8). The catalytic residues are arranged in three conserved blocks spread widely through the amino acid sequence (Pons et al. Proteins: Structure, Function and Bioinformatics, 2004, 54:424-432). Among the GH43 family enzymes tested for useful activities in biomass hydrolysis, the predicted catalytic residues are shown as the bold and underlined residues in the sequences of FIG. 93. The crystal structure of the G. stearothermophylus xylosidase (Brux et al. J. Mol. Bio., 2006, 359:97-109) suggests several additional residues that may be important for substrate binding in this enzyme. Because the GH43 family enzymes tested for biomass hydrolysis had differing substrate preferences, these residues are not fully conserved in the sequences aligned in FIG. 93. However among the xylosidases tested, several conserved residues that contribute to substrate binding, either through hydrophobic interaction or through hydrogen bonding, are conserved and are noted by single underlines in FIG. 93.

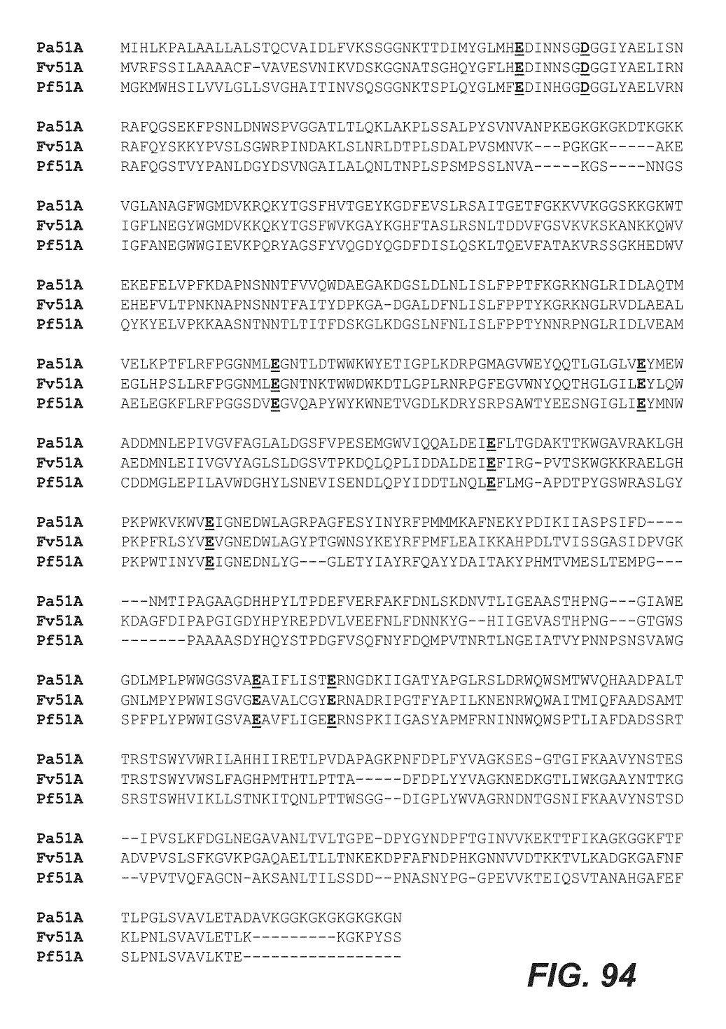

Glycoside hydrolase family 51 (“GH51”) enzymes have L-α-arabinofuranosidase (EC 3.2.1.55) and/or endoglucanase (EC 3.2.1.4) activity. High-resolution crystal structure of a GH51 L-α-arabinofuranosidase from G.s stearothermophilus T-6 shows that the enzyme is a hexamer, with each monomer organized into two domains: an 8-barrel (β/α) and a 12-stranded β sandwich with jelly-roll topology (see Hovel et al. EMBO J. 2003, 22(19):4922-4932). It can be expected that the catalytic residues will be acidic and conserved across enzyme sequences in the family. When the amino acid sequences of Fv51A, Pf51A, and Pa51A are aligned with GH51 enzymes of more diverse sequence, 8 acidic residues remain conserved. Those are shown bold and underlined in FIG. 94.

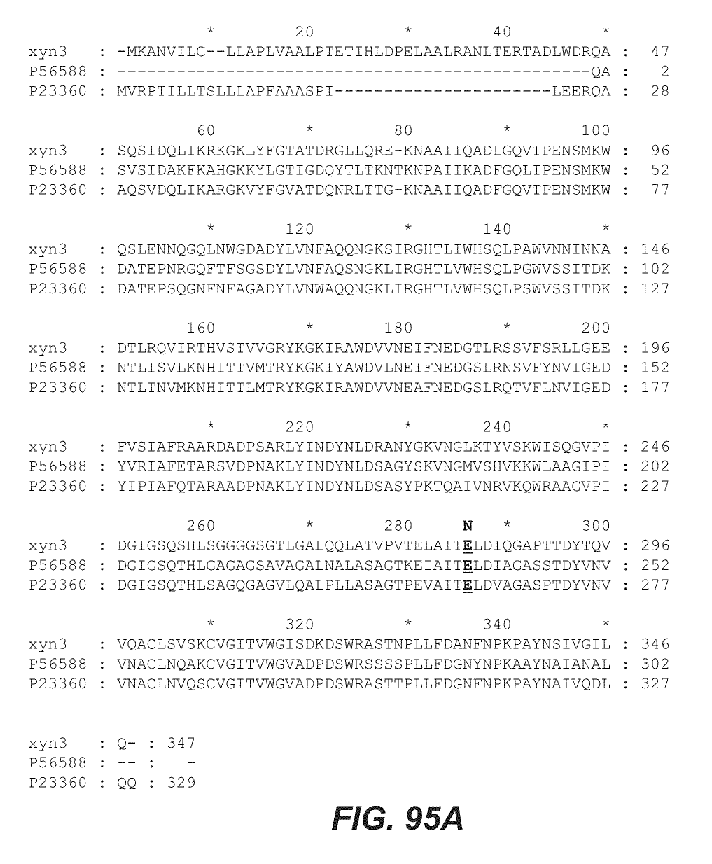

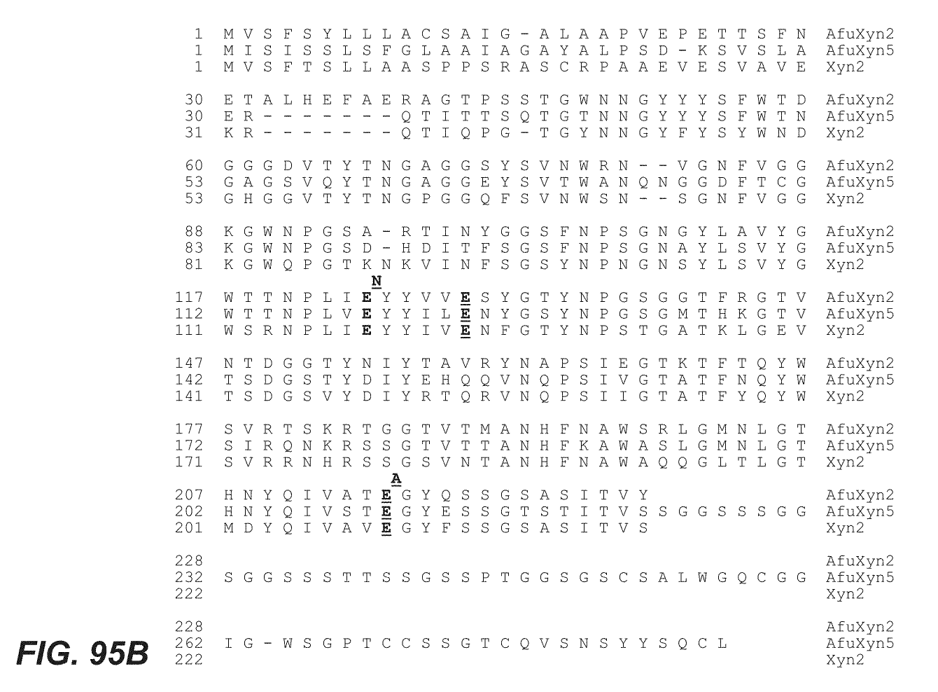

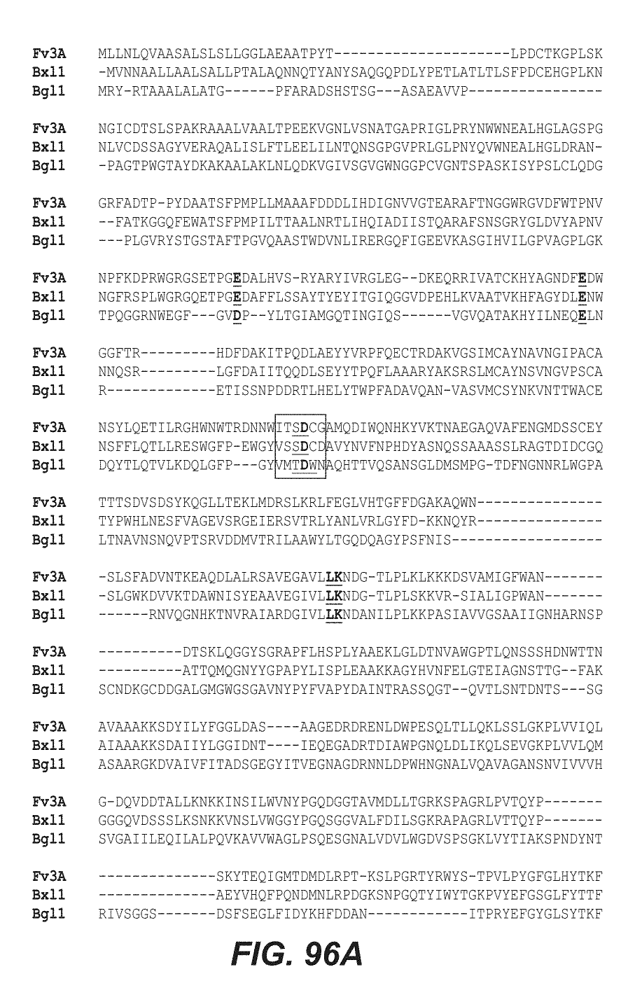

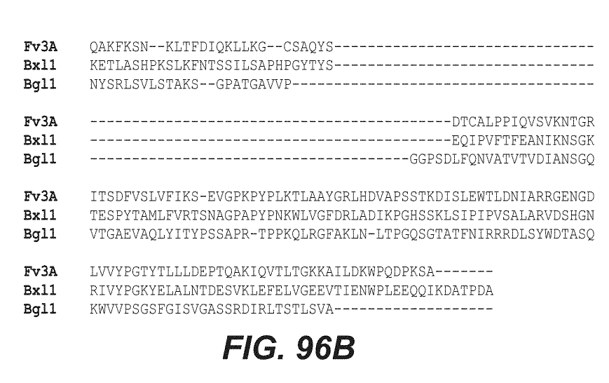

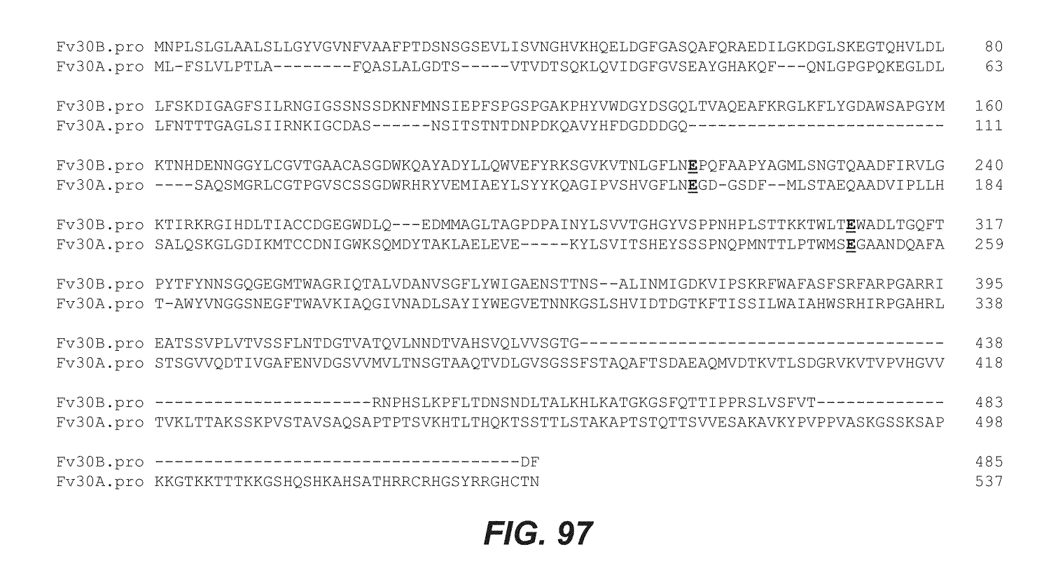

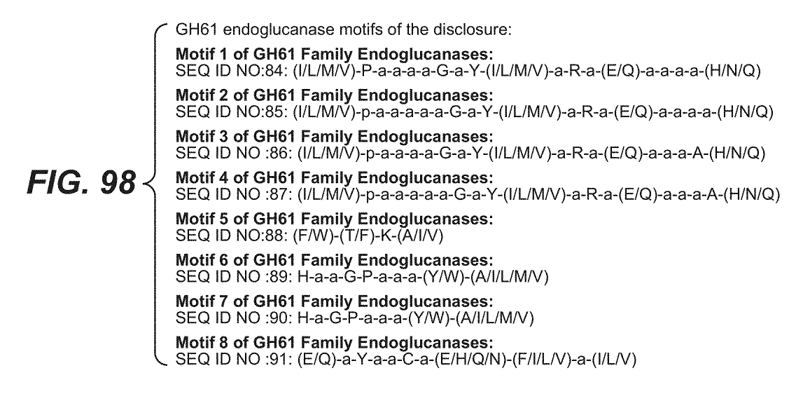

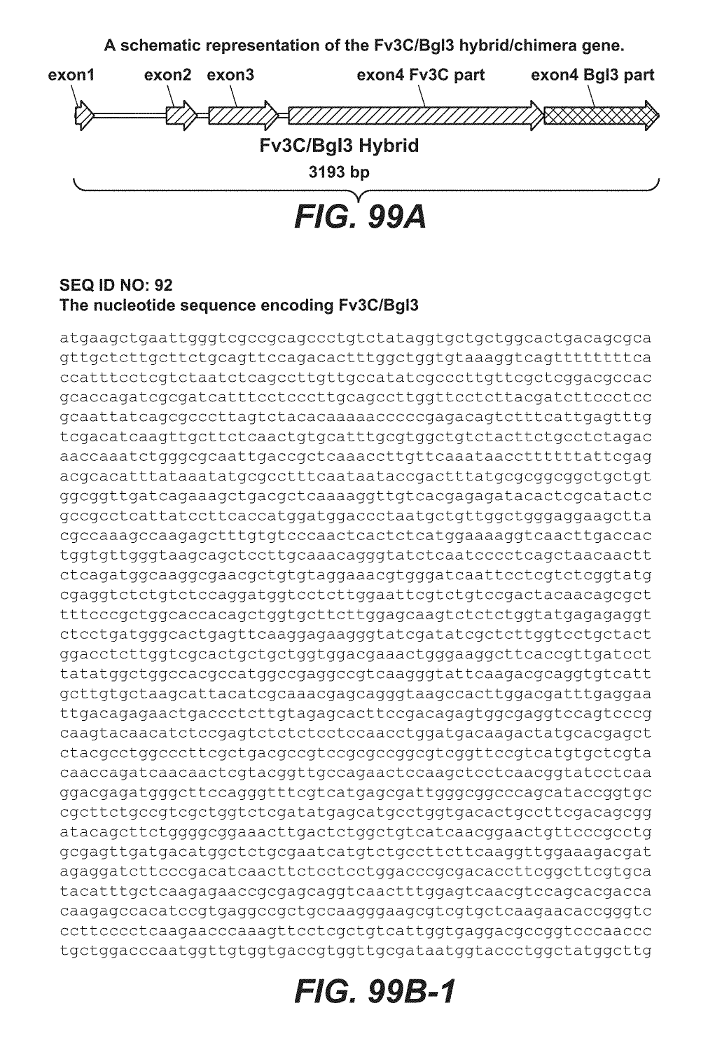

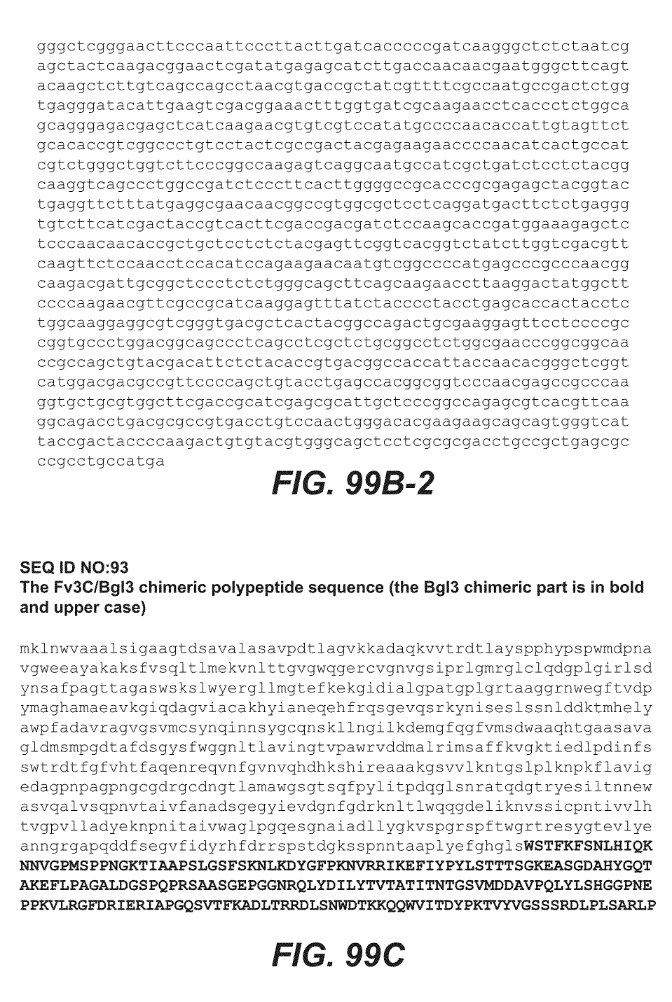

Glycoside hydrolase family 10 (“GH10”) enzymes also have an 8-barrel (β/α) structure. They hydrolyze in an endo fashion with a retaining mechanism that uses at least one acidic catalytic residue in a generally acid/base catalysis process (Pell et al., J. Biol. Chem., 2004, 279(10): 9597-9605). Crystal structures of the GH10 xylanases of P. simplicissimum (Uniprot P56588) and T. aurantiacus (Uniprot P23360) complexed with substrates in the active sites have been solved (see Schmidt et al. Biochem., 1999, 38:2403-2412; and Lo Leggio et al. FEBS Lett. 2001, 509: 303-308). T. reesei Xyn3 residues that are important for substrate binding and catalysis can be derived from an alignment with the sequences of abovementioned GH10 xylanases from P. simplicissimum and T. aurantiacus (FIG. 95A). T. reesei Xyn3 residue E282 is predicted to be the catalytic nucleophilic residue, whereas residues E91, N92, K95, Q97, S98, H128, W132, Q135, N175, E176, Y219, Q252, H254, W312, and/or W320 are predicted to be involved in substrate binding and/or catalysis.

Glycoside hydrolase family 11 (“GH11”) enzymes have a β-jelly roll structure. They hydrolyze in an endo fashion with a retaining mechanism that uses at least one acidic catalytic residue in a generally acid/base catalysis process. Several other residues spread throughout their structure may contribute to stabilizing the xylose units in the substrate neighboring the pair of xylose monomers that are cleaved by hydrolysis. Three GH11 family endoxylanases were tested and their sequences are aligned in FIG. 95B. E118 (or E86 in mature T. reesei Xyn2) and E209 (or E177 in mature T. reesei Xyn2) have been identified as catalytic nucleophile and general/acid base residues in T. reesei Xyn2, respectively (see Havukainen et al. Biochem., 1996, 35:9617-24).

Glycoside hydrolase family 30 (“GH30”) enzymes are retaining enzymes having glucosylceramidase (EC 3.2.1.45); β-1,6-glucanase (EC 3.2.1.75); β-xylosidase (EC 3.2.1.37); β-glucosidase (3.2.1.21) activity. The first GH30 crystal structure was the Gaucher disease-related human β-glucocerebrosidase solved by Grabowski, et al. (Crit Rev Biochem Mol Biol 1990; 25(6) 385-414). GH30 have an (α/β) μM barrel fold with the two key active site glutamic acids located at the C-terminal ends of β-strands 4 (acid/base) and 7 (nucleophile) (Henrissat B, et al. Proc Natl Acad Sci USA, 92(15):7090-4, 1995; Jordan et al., Applied Microbiol Biotechnol, 86:1647, 2010). Glutamate 162 of Fv30A is conserved in 14 of 14 aligned GH30 proteins (13 bacterial proteins and one endo-b-xylanase from the fungi Biospora accession no. ADG62369) and glutamate 250 of Fv30A is conserved in 10 of the same 14, is an aspartate in another three and non-acidic in one. There are other moderately conserved acidic residues but no others are as widely conserved.

Glycoside hydrolase 61 (“GH61”) enzymes have been identified in Eukaryota. A weak endo-glucanase activity has been observed for Cel61A from H. jecorina (Karlsson et al, Eur J Biochem, 2001, 268(24):6498-6507). GH61 polypeptides potentiate the enzymatic hydrolysis of lignocellulosic substrates by cellulases (Harris et al, 2010, Biochemistry, 49(15),3305-16). Studies on homologous polypeptides involved in chitin degradation predict that GH61 polypeptides employ an oxidative hydrolysis mechanism that requires an electron donor substrate and in which divalent metal ions are involved (Vaaje-Kolstad, 2010, Science, 330(6001), 219-22). This agrees with the observation that the synergistic effect of GH61 polypeptides on lignocellulosic substrate degradation is dependent on divalent ions (Harris et al, 2010, Biochemistry, 49(15), 3305-16). In addition, the available structures of GH61 polypeptides have divalent atoms bound by a number of fully conserved amino acid residues (Karkehabadi, 2008, J. Mol. Biol., 383(1), 144-54; Harris et al, 2010, Biochemistry, 49(15),3305-16). The GH61 polypeptides have a flat surface at the metal binding site that is formed by conserved residues and might be involved in substrate binding (Karkehabadi, 2008, J. Mol. Biol., 383(1), 144-54).

The term “isolated” as used herein with nucleic acids, such as DNA or RNA, refers to molecules separated from other DNAs or RNAs, respectively, which are present in the natural source of the nucleic acid. Moreover, by an “isolated nucleic acid” is meant to include nucleic acid fragments, which are not naturally occurring as fragments and would not be found in the natural state. The term “isolated” when used with polypeptides refers to those isolated from other cellular proteins, or to purified and recombinant polypeptides. The term “isolated” also refers to a nucleic acid or peptide that is substantially free of cellular material, viral material, or culture medium when produced by recombinant DNA techniques.

The term “isolated” as used herein also refers to a nucleic acid or peptide that is substantially free of chemical precursors or other chemicals when chemically synthesized. Unless defined otherwise, all technical and scientific terms used herein have the same meaning as commonly understood by one of ordinary skill in the art to which this invention belongs. Singleton, et al., DICTIONARY OF MICROBIOLOGY AND MOLECULAR BIOLOGY, 2D ED., John Wiley and Sons, New York (1994), and Hale & Marham, THE HARPER COLLINS DICTIONARY OF BIOLOGY, Harper Perennial, N.Y. (1991) provide one of skill with a general dictionary of many of the terms used in this invention. Although any methods and materials similar or equivalent to those described herein can be used in the practice or testing of the present invention, the preferred methods and materials are described. Numeric ranges are inclusive of the numbers defining the range. It is to be understood that this invention is not limited to the particular methodology, protocols, and reagents described, as these may vary.

The headings provided herein are not limitations of the various aspects or embodiments of the invention which can be had by reference to the specification as a whole. Accordingly, the terms defined immediately below are more fully defined by reference to the specification as a whole.

The disclosure provides compositions comprising a polypeptide having glycosyl hydrolase family 61 (“GH61”)/endoglucanase activity, nucleotides encoding a polypeptide provided, vectors containing a nucleotide provided, and cells containing a nucleotide and/or vector provided. The disclosure also provides methods of hydrolyzing a biomass material and/or reducing the viscosity of a biomass mixture using a composition provided.

As used herein, a “variant” of polypeptide X refers to a polypeptide having the amino acid sequence of polypeptide X in which one or more amino acid residues are altered. The variant may have conservative or nonconservative changes. Guidance in determining which amino acid residues may be substituted, inserted, or deleted without affecting biological activity may be found using computer programs well known in the art, for example, LASERGENE software (DNASTAR). A variant of the invention includes polypeptides comprising altered amino acid sequences in comparison with a precursor enzyme amino acid sequence, wherein the variant enzyme retains the characteristic cellulolytic nature of the precursor enzyme but may have altered properties in some specific aspects, for example, an increased or decreased pH optimum, an increased or decreased oxidative stability; an increased or decreased thermal stability, and increased or decreased level of specific activity towards one or more substrates, as compared to the precursor enzyme.

The term “variant,” when used in the context of a polynucleotide sequence, may encompass a polynucleotide sequence related to that of a gene or the coding sequence thereof. This definition may also include, e.g., “allelic,” “splice,” “species,” or “polymorphic” variants. A splice variant may have significant identity to a reference polynucleotide, but will generally have a greater or fewer number of residues due to alternative splicing of exons during mRNA processing. The corresponding polypeptide may possess additional functional domains or an absence of domains. Species variants are polynucleotide sequences that vary from one species to another. The resulting polypeptides generally will have significant amino acid identity relative to each other. A polymorphic variant is a variation in the polynucleotide sequence of a particular gene between individuals of a given species.

As used herein, a “mutant” of polypeptide X refers to a polypeptide wherein one or more amino acid residues have undergone an amino acid substitution while retaining the native enzymatic activity (i.e., the ability to catalyze certain hydrolysis reactions). As such, a mutant X polypeptide constitutes a particular type of X polypeptide, as that term is defined herein. Mutant X polypeptides can be made by substituting one or more amino acids into the native or wild type amino acid sequence of the polypeptide. In some aspects, the invention includes polypeptides comprising altered amino acid sequences in comparison with a precursor enzyme amino acid sequence, wherein the mutant enzyme retains the characteristic cellulolytic or hemicelluloytic nature of the precursor enzyme but may have altered properties in some specific aspects, e.g., an increased or decreased pH optimum, an increased or decreased oxidative stability; an increased or decreased thermal stability, and increased or decreased level of specific activity towards one or more substrates, as compared to the precursor enzyme. Guidance in determining which amino acid residues may be substituted, inserted, or deleted without affecting biological activity may be found using computer programs well known in the art, for example, LASERGENE software (DNASTAR). The amino acid substitutions may be conservative or non-conservative and such substituted amino acid residues may or may not be one encoded by the genetic code.

The amino acid substitutions may be located in the polypeptide carbohydrate-binding domains (CBMs), in the polypeptide catalytic domains (CD), and/or in both the CBMs and the CDs. The standard twenty amino acid “alphabet” has been divided into chemical families based on similarity of their side chains. Those families include amino acids with basic side chains (e.g., lysine, arginine, histidine), acidic side chains (e.g., aspartic acid, glutamic acid), uncharged polar side chains (e.g., glycine, asparagine, glutamine, serine, threonine, tyrosine, cysteine), nonpolar side chains (e.g., alanine, valine, leucine, isoleucine, proline, phenylalanine, methionine, tryptophan), beta-branched side chains (e.g., threonine, valine, isoleucine) and aromatic side chains (e.g., tyrosine, phenylalanine, tryptophan, histidine). A “conservative amino acid substitution” is one in which the amino acid residue is replaced with an amino acid residue having a chemically similar side chain (i.e., replacing an amino acid having a basic side chain with another amino acid having a basic side chain). A “non-conservative amino acid substitution” is one in which the amino acid residue is replaced with an amino acid residue having a chemically different side chain (i.e., replacing an amino acid having a basic side chain with another amino acid having an aromatic side chain).

As used herein, a polypeptide or nucleic acid that is “heterologous” to a host cell refers to a polypeptide or nucleic acid that does not naturally occur in a host cell.

Reference to “about” a value or parameter herein includes (and describes) variations that are directed to that value or parameter per se. For example, description referring to “about X” includes description of “X”.

As used herein and in the appended claims, the singular forms “a,” “or,” and “the” include plural referents unless the context clearly dictates otherwise.

It is understood that aspects and variations of the methods and compositions described herein include “consisting” and/or “consisting essentially of” aspects and variations. The term “comprising” is broader than “consisting” or “consisting essentially of.”

As used herein, the term “operably linked” means that selected nucleotide sequence (e.g., encoding a polypeptide described herein) is in proximity with a regulatory sequence, e.g., a promoter, to allow the sequence to regulate expression of the selected DNA. For example, the promoter is located upstream of the selected nucleotide sequence in terms of the direction of transcription and translation. By “operably linked” is meant that a nucleotide sequence and a regulatory sequence(s) are connected in such a way as to permit gene expression when the appropriate molecules (e.g., transcriptional activator proteins) are bound to the regulatory sequence(s).

As used herein, the term “hybridizes under low stringency, medium stringency, high stringency, or very high stringency conditions” describes conditions for hybridization and washing. Guidance for performing hybridization reactions can be found in Current Protocols in Molecular Biology, John Wiley & Sons, N.Y. (1989), 6.3.1-6.3.6. Aqueous and nonaqueous methods are described in that reference and either method can be used. Specific hybridization conditions referred to herein are as follows: 1) low stringency hybridization conditions in 6× sodium chloride/sodium citrate (SSC) at about 45° C., followed by two washes in 0.2×SSC, 0.1% SDS at least at 50° C. (the temperature of the washes can be increased to 55° C. for low stringency conditions); 2) medium stringency hybridization conditions in 6×SSC at about 45° C., followed by one or more washes in 0.2×SSC, 0.1% SDS at 60° C.; 3) high stringency hybridization conditions in 6×SSC at about 45° C., followed by one or more washes in 0.2.×SSC, 0.1% SDS at 65° C.; and preferably 4) very high stringency hybridization conditions are 0.5M sodium phosphate, 7% SDS at 65° C., followed by one or more washes at 0.2×SSC, 1% SDS at 65° C. Very high stringency conditions (4) are the preferred conditions unless otherwise specified.

5.1 Polypeptides of the Disclosure

The disclosure provides isolated, synthetic or recombinant polypeptides comprising an amino acid sequence having at least about 60% (e.g., at least about 60%, 65%, 70%, 75%, 80%, 85%, 90%, 91%, 92%, 93%, 94%, 95%, 96%, 97%, 98%, 99%, or 100%) identity to any one of SEQ ID NOs: 54, 56, 58, 60, 62, 64, 66, 68, 70, 72, 74, 76, 78, 79, 93, and 95, over a region of at least about 10 (e.g., at least about 10, 15, 20, 25, 30, 35, 40, 45, 50, 55, 60, 65, 70, 75, 80, 85, 90, 95, 100, 125, 150, 175, 200, 225, 250, 275, 300) residues, or over the full length catalytic domain (CD) or the full length carbohydrate binding domain (CBM). The isolated, synthetic, or recombiant polypeptides can have β-glucosidase activity. In certain embodiments, the isolated, synthetic, or recombinant polypeptides are β-glucosidase polypeptides, which include, e.g., variants, mutants, and hybrid/chimeric β-glucosidase polypeptides. In certain embodiments, the disclosure provides a polypeptide having β-glucosidase activity that is a hybrid/chimera of two or more β-glucosidase sequences, wherein the first of the two or more β-glucosidase sequences is at least about 200 (e.g., at least about 200, 250, 300, 350, 400, or 500) amino acid residues in length and comprises one or more or all of the amino acid sequence motifs of SEQ ID NOs: 96-108, the second of the two or more β-glucosidase sequences is at least about 50 (e.g., at least about 50, 75, 100, 125, 150, 175, or 200) amino acid residues in length and comprises one or more or all of the amino acid sequence motifs of SEQ ID NOs: 109-116. In particular, the first of the two or more β-glucosidase sequences is one that is at least about 200 amino acid residues in length and comprises at least 2 (e.g., at least 2, 3, 4, or all) of the amino acid sequence motifs of SEQ ID NOs: 197-202, and the second of the two or more β-glucosidase is at least 50 amino acid residues in length and comprises SEQ ID NO:203. In some embodiments, the first sequence is located at the N-terminal of the chimeric/hybrid β-glucosidase polypeptide, whereas the second sequence is located at the C-terminal of the chimeric/hybrid β-glucosidase polypeptide. In some embodiments, the first sequence is connected by its C-terminus to the second sequence by its N-terminus. For example, the first sequence is immediately adjacent or directly connected to the second sequence.

Alternatively, the first sequence is not immediately adjacent to the second sequence, but rather the first and the second sequences are connected via a linker domain. In certain embodiments, the first sequence, the second sequence, or both the first and the second sequences comprise 1 or more glycosylation sites. In some embodiments, either the first or the second sequence comprises a loop sequence or a sequence that encodes a loop-like structure. In certain embodiments, the loop sequence is about 3, 4, 5, 6, 7, 8, 9, 10, or 11 amino acid residues in length, comprising an amino acid sequence of FDRRSPG (SEQ ID NO:204), or of FD(R/K)YNIT (SEQ ID NO:205). In certain embodiments, neither the first nor the second sequence comprises a loop sequence, rather the linker domain connecting the first and the second sequences comprise such a loop sequence. The hybrid/chimeric β-glucosidase polypeptide has improved stability as compared to the counterpart β-glucosidase from which each of the first, second, or the linker domain sequences is derived.

In some embodiments, the improved stability is an improved proteolytic stability or resistance to proteolytic cleavage during storage under storage under standard conditions, or during expression and/or production, under standard expression/production conditions, e.g., from proteolytic cleavage at a residue in the loop sequence, or at a residue that is outside the loop sequence.

In certain aspects, the disclosure provides an isolated, synthetic, or recombinant β-glucosidase polypeptide, which is a hybrid of at least 2 (e.g., 2, 3, or even 4) β-glucosidase sequences, wherein the first of the at least 2 β-glucosidase sequences is one that is at least about 200 (e.g., at least about 200, 250, 300, 350, or 400) amino acid residues in length and comprises a sequence that has at least about 60% (e.g., at least about 65%, 70%, 75%, 80%, 85%, 90%, 91%, 92%, 93%, 94%, 95%, 96%, 97%, 98%, 99%, or 100%) identity to a sequence of equal length of any one of SEQ ID NOs: 54, 56, 58, 62, 64, 66, 68, 70, 72, 74, 76, 78, and 79, whereas the second of the at least 2 β-glucosidase sequences is one that is at least about 50 (e.g., at least about 50, 75, 100, 125, 150, or 200) amino acid residues in length and comprises a sequence that has at least about 60% (e.g., at least about 65%, 70%, 75%, 80%, 85%, 90%, 91%, 92%, 93%, 94%, 95%, 96%, 97%, 98%, 99%, or 100%) identity to a sequence of equal length of SEQ ID NO:60. The disclosure also provides an isolated, synthetic, or recombinant polypeptide having β-glucosidase activity, which is a hybrid of at least 2 (e.g., 2, 3, or even 4) β-glucosidase sequences, wherein the first of the at least 2 β-glucosidase sequences is one that is at least about 200 amino acid residues in length and comprises a sequence that has at least about 60% identity to a sequence of equal length of SEQ ID NO:60, whereas the second of the at least 2 β-glucosidase sequences is one that is at least about 50 amino acid residues in length and comprises a sequence that has at least about 60% identity to a sequence of equal length of any one of SEQ ID NOs: 54, 56, 58, 62, 64, 66, 68, 70, 72, 74, 76, 78, and 79. In particular, the first of the two or more β-glucosidase sequences is one that is at least about 200 amino acid residues in length and comprises at least 2 (e.g., at least 2, 3, 4, or all) of the amino acid sequence motifs of SEQ ID NOs: 197-202, and the second of the two or more β-glucosidase is at least 50 amino acid residues in length and comprises SEQ ID NO:203. In some embodiments, the first sequence is located at the N-terminal of the chimeric or hybrid β-glucosidase polypeptide, whereas the second sequence is located at the C-terminal of the chimeric or hybrid β-glucosidase polypeptide. In some embodiments, the first sequence is connected by its C-terminus to the second sequence by its N-terminus, e.g., the first sequence is adjacent or directly connected to the second sequence. Alternatively, the first sequence is not adjacent to the second sequence, but rather the first sequence is connected to the second sequence via a linker domain. The first sequence, the second sequence, or both the first and the second sequences can comprise 1 or more glycosylation sites. The first or the second sequence can comprise a loop sequence or a sequence that encodes a loop-like structure, derived from a third β-glucosidase polypeptide, is about 3, 4, 5, 6, 7, 8, 9, 10, or 11 amino acid residues in length, comprising an amino acid sequence of FDRRSPG (SEQ ID NO:204), or of FD(R/K)YNIT (SEQ ID NO:205). In certain embodiments, neither the first nor the second sequence comprises a loop sequence, rather, the linker domain connecting the first and the second sequences comprise such a loop sequence. In some embodiments, the hybrid/chimeric β-glucosidase polypeptide has improved stability as compared to the counterpart β-glucosidase polypeptide from which each of the first, the second, or the linker domain sequences is derived. In some embodiments, the improved stability is an improved proteolytic stability, rendering the fusion/chimeric polypeptide less susceptible to proteolytic cleavage at either a residue in the loop sequence or at a residue or position that is outside the loop sequence, during storage under standard storage conditions, or during expression and/or production, under standard expression/production conditions.

In certain aspects, the disclosure provides a fusion/chimeric β-glucosidase polypeptide derived from 2 or more β-glucosidase sequences, wherein the first sequence is derived from Fv3C and is at least about 200 amino acid residues in length, and the second sequence is derived from T. reesei Bgl3 (or “Tr3B”), and is at least about 50 amino acid residues in length. In some embodiments, the C-terminus of the first sequence is connected to the N-terminus of the second sequence such that the first sequence is immediately adjacent or directly connected to the second sequence. Alternatively, the first sequence is connected to the second sequence via a linker domain. In some embodiments, either the first or the second sequence comprises a loop sequence derived from a third β-glucosidase polypeptide, which is about 3, 4, 5, 6, 7, 8, 9, 10, or 11 amino acid residues in length, and comprising an amino acid sequence of FDRRSPG (SEQ ID NO:204), or of FD(R/K)YNIT (SEQ ID NO:205). In certain embodiments, the linker domain connecting the first and the second sequence comprises the loop sequence. In certain embodiments, the loop sequence is derived from Te3A. In some embodiments, the fusion/chimeric β-glucosidase polypeptide has improved stability as compared to its counterpart β-glucosidase polypeptide from which each of the chimeric parts is derived, e.g., over that of Fv3C, Te3A, and/or Tr3B.

In some embodiments, the improved stability is an improved proteolytic stability, rendering the fusion/chimeric polypeptide less susceptible to proteolytic cleavage at either a residue in the loop sequence or at a residue or position that is outside the loop sequence during storage under standard storage conditions, or during expression and/or production, under standard expression/production conditions. For example, the fusion/chimeric polypeptide is less susceptible to proteolytic cleavage at a residue upstream to the C-terminus of the loop sequence as compared to an Fv3C polypeptide at the same position when, e.g., the sequences of the chimera and the Fv3C polypeptides are aligned.

The disclosure also provides isolated, synthetic or recombinant polypeptides having β-glucosidase activity comprising an amino acid sequence having at least about 60% (e.g., at least about 60%, 65%, 70%, 75%, 80%, 85%, 90%, 91%, 92%, 93%, 94%, 95%, 96%, 97%, 98%, 99%, or 100%) sequence identity to any one of SEQ ID NOs: 54, 56, 58, 60, 62, 64, 66, 68, 70, 72, 74, 76, 78, 79, 93, and 95, or over the full length catalytic domain (CD) or the full length carbohydrate binding domain (CBM).

In some aspects, the disclosure provides isolated, synthetic or recombinant polypeptides comprising an amino acid sequence having at least about 60% (e.g., at least about 60%, 65%, 70%, 75%, 80%, 85%, 90%, 91%, 92%, 93%, 94%, 95%, 96%, 97%, 98%, 99%, or 100%) identity to any one of SEQ ID NOs: 52, 80-81, 206-207, over a region of at least about 10 (e.g., at least about 10, 15, 20, 25, 30, 35, 40, 45, 50, 55, 60, 65, 70, 75, 80, 85, 90, 95, 100, 125, 150, 175, 200, 225, 250, 275, 300) residues, or over the full length catalytic domain (CD) or carbohydrate binding domain (CBM). In certain embodiments, the isolated, synthetic, or recombiant polypeptides have GH61/endoglucanase activity. The disclosure also provides isolated, synthetic or recombinant polypeptides comprising an amino acid sequence of at least about 50 (e.g., at least about 50, 100, 150, 200, 250, or 300) amino acid residues in length, comprising one or more of the sequence motifs selected from the group consisting of (1) SEQ ID NOs:84 and 88; (2) SEQ ID NOs:85 and 88; (3) SEQ ID NO:86; (4) SEQ ID NO:87; (5) SEQ ID NOs:84, 88 and 89; (6) SEQ ID NOs:85, 88, and 89; (7) SEQ ID NOs: 84, 88, and 90; (8) SEQ ID NOs: 85, 88 and 90; (9) SEQ ID NOs:84, 88 and 91; (10) SEQ ID NOs: 85, 88 and 91; (11) SEQ ID NOs: 84, 88, 89 and 91; (12) SEQ ID NOs: 84, 88, 90 and 91; (13) SEQ ID NOs: 85, 88, 89 and 91: and (14) SEQ ID NOs: 85, 88, 90 and 91. In certain embodiments, the polypeptide is a GH61 endoglucanase polypeptide, e.g., an EG IV polypeptide from a suitable microorganism, such as T. reesei Eg4). In some embodiments, the GH61 endoglucanase polypeptide is a variant, a mutant or a fusion polypeptide derived from T. reesei Eg4 (e.g., a polypeptide comprising at least about 60%, 65%, 70%, 75%, 80%, 85%, 90%, 91%, 92%, 93%, 94%, 95%, 96%, 97%, 98%, 99%, or 100% sequence identity to SEQ ID NO:52).

The disclosure also provides an isolated, synthetic, or recombinant polypeptide having at least about 70%, e.g., at least about 71%, 72%, 73%, 74%, 75%, 76%, 77%, 78%, 79%, 80%, 81%, 82%, 83%, 84%, 85%, 86%, 87%, 88%, 89%, 90%, 91%, 92%, 93%, 94%, 95%, 96%, 97%, 98%, or 99%, or complete (100%) identity to a polypeptide of any one of SEQ ID NOs:2, 4, 6, 8, 10, 12, 14, 16, 18, 20, 22, 24, 26, 28, 30, 32, 34, 36, 38, 40, 42, 43, and 45, over a region of at least about 10, e.g., at least about 15, 20, 25, 30, 35, 40, 45, 50, 55, 60, 65, 70, 75, 80, 85, 90, 95, 100, 125, 150, 175, 200, 225, 250, 275, 300, 325, or 350 residues, or over the full length immature polypeptide, the full length mature polypeptide, the full length catalytic domain (CD) or carbohydrate binding domain (CBM).

The disclosure provides, in some aspects, isolated, synthetic, or recombinant nucleotides encoding a β-glucosidase polypeptide having at least 60% (e.g., at least about 60%, 65%, 70%, 75%, 80%, 85%, 90%, 91%, 92%, 93%, 94%, 95%, 96%, 97%, 98%, 99%, or 100%) sequence identity to any one of SEQ ID NOs: 54, 56, 58, 60, 62, 64, 66, 68, 70, 72, 74, 76, 78, 79, 93, and 95, over a region of at least about 10 (e.g., at least about 10, 15, 20, 25, 30, 35, 40, 45, 50, 55, 60, 65, 70, 75, 80, 85, 90, 95, 100, 125, 150, 175, 200, 225, 250, 275, 300) residues, or over the full length catalytic domain (CD) or carbohydrate binding domain (CBM). In some embodiments, the isolated, synthetic, or recombinant nucleotide encodes a fusion/chimeric polypeptide having β-glucosidase activity comprising a first sequence of at least about 200 (e.g., at least about 200, 250, 300, 350, 400, or 500) amino acid residues in length and comprises one or more or all of the amino acid sequence motifs of SEQ ID NOs: 96-108, a second sequence that is at least about 50 (e.g., at least about 50, 75, 100, 125, 150, 175, or 200) amino acid residues in length and comprises one or more or all of the amino acid sequence motifs of SEQ ID NOs: 109-116. In particular, the first of the two or more β-glucosidase sequences is one that is at least about 200 amino acid residues in length and comprises at least 2 (e.g., at least 2, 3, 4, or all) of the amino acid sequence motifs of SEQ ID NOs: 197-202, and the second of the two or more β-glucosidase is at least 50 amino acid residues in length and comprises SEQ ID NO:203. In certain embodiments, the C-terminus of the first sequence is connected to the N-terminus of the second sequence.

In other embodiments, the first and the second β-glucosidase sequences are connected via a linker domain, which can comprise a loop sequence, which is about 3, 4, 5, 6, 7, 8, 9, 10, or 11 amino acid residues in length, and is derived from a third β-glucosidase polypeptide, comprising an amino acid sequence of FDRRSPG (SEQ ID NO:204), or of FD(R/K)YNIT (SEQ ID NO:205).

In certain aspects, the disclosure provides an isolated, synthetic, or recombinant nucleotide encoding a β-glucosidase polypeptide, which is a hybrid of at least 2 (e.g., 2, 3, or even 4) β-glucosidase sequences, wherein the first β-glucosidase sequences is one that is at least about 200 (e.g., at least about 200, 250, 300, 350, or 400) amino acid residues in length and comprises a sequence that has at least about 60% (e.g., at least about 65%, 70%, 75%, 80%, 85%, 90%, 91%, 92%, 93%, 94%, 95%, 96%, 97%, 98%, 99%, or 100%) identity to a sequence of equal length of any one of SEQ ID NOs: 54, 56, 58, 62, 64, 66, 68, 70, 72, 74, 76, 78, and 79, whereas the second β-glucosidase sequences is one that is at least about 50 (e.g., at least about 50, 75, 100, 125, 150, or 200) amino acid residues in length and comprises a sequence that has at least about 60% (e.g., at least about 65%, 70%, 75%, 80%, 85%, 90%, 91%, 92%, 93%, 94%, 95%, 96%, 97%, 98%, 99%, or 100%) identity to a sequence of equal length of SEQ ID NO:60. The disclosure also provides an isolated, synthetic, or recombinant nucleotide encoding a polypeptide having β-glucosidase activity, which is a hybrid or fusion of at least 2 (e.g., 2, 3, or even 4) β-glucosidase sequences, wherein the first sequences is one that is at least about 200 (e.g., at least about 200, 250, 300, 350, or 400) amino acid residues in length and comprises a sequence that has at least about 60% (e.g., at least about 65%, 70%, 75%, 80%, 85%, 90%, 91%, 92%, 93%, 94%, 95%, 96%, 97%, 98%, 99%, or 100%) identity to a sequence of equal length of SEQ ID NO:60, whereas the second sequences is one that is at least about 50 (e.g., at least about 50, 75, 100, 125, 150, or 200) amino acid residues in length and comprises a sequence that has at least about 60% (e.g., at least about 65%, 70%, 75%, 80%, 85%, 90%, 91%, 92%, 93%, 94%, 95%, 96%, 97%, 98%, 99%, or 100%) identity to a sequence of equal length of any one of SEQ ID NOs: 54, 56, 58, 62, 64, 66, 68, 70, 72, 74, 76, 78, and 79. In particular, the first of the two or more β-glucosidase sequences is one that is at least about 200 amino acid residues in length and comprises at least 2 (e.g., at least 2, 3, 4, or all) of the amino acid sequence motifs of SEQ ID NOs: 197-202, and the second of the two or more β-glucosidase is at least 50 amino acid residues in length and comprises SEQ ID NO:203. In some embodiments, the nucleotide encodes a first amino acid sequence, located at the N-terminal of the chimeric/fusion β-glucosidase polypeptide, and a second amino acid sequence located at the C-terminal of the chimeric/fusion β-glucosidase polypeptide, wherein the C-terminus of the first sequence is connected to the N-terminus of the second sequence. Alternatively, the first sequence is connected to the second sequence via a linker domain. In some embodiments, the first amino acid sequence, the second amino acid sequence, or the linker domain comprises an amino acid sequence comprising a sequence that represents a loop-like structure, derived from a third β-glucosidase polypeptide, is about 3, 4, 5, 6, 7, 8, 9, 10, or 11 amino acid residues in length, and comprising an amino acid sequence of FDRRSPG (SEQ ID NO:204), or of FD(R/K)YNIT (SEQ ID NO:205).

In some aspects, the disclosure provides isolated, synthetic, or recombinant nucleotides having at least 60% (e.g., at least about 60%, 65%, 70%, 75%, 80%, 85%, 90%, 91%, 92%, 93%, 94%, 95%, 96%, 97%, 98%, 99%, or 100%) identity to any one of SEQ ID NOs: 52, 55, 57, 59, 61, 63, 65, 67, 69, 71, 73, 75, 77, 92 or 94, or to a fragment thereof of at least about 300 (e.g., at least about 300, 400, 500, or 600) residues in length. In certain embodiments, the disclosure provides isolated, synthetic, or recombinant nucleotides that are capable of hybridizing to any one of SEQ ID NOs: 53, 55, 57, 59, 61, 63, 65, 67, 69, 71, 73, 75, 77, 92 or 94, to a fragment of at least about 300 residues in length, or to a complement thereof, under low stringency, medium stringency, high stringency, or very high stringency conditions.

The disclosure also provides, in certain aspects, an isolated, synthetic, or recombinant nucleotide encoding a polypeptide having GH61/endoglucanase activity comprising an amino acid sequence having at least about 60% (e.g., at least about 60%, 65%, 70%, 75%, 80%, 85%, 90%, 91%, 92%, 93%, 94%, 95%, 96%, 97%, 98%, 99%, or 100%) identity to any one of SEQ ID NOs: 52, 80-81, 206-207, over a region of at least about 10 (e.g., at least about 10, 15, 20, 25, 30, 35, 40, 45, 50, 55, 60, 65, 70, 75, 80, 85, 90, 95, 100, 125, 150, 175, 200, 225, 250, 275, 300) residues, or over the full length catalytic domain (CD) or carbohydrate binding domain (CBM). In some embodiments, the disclosure provides an isolated, synthetic or recombinant encoding a polypeptide comprising an amino acid sequence of at least about 50 (e.g., at least about 50, 100, 150, 200, 250, or 300) amino acid residues in length, comprising one or more of the sequence motifs selected from the group consisting of (1) SEQ ID NOs:84 and 88; (2) SEQ ID NOs:85 and 88; (3) SEQ ID NO:86; (4) SEQ ID NO:87; (5) SEQ ID NOs:84, 88 and 89; (6) SEQ ID NOs:85, 88, and 89; (7) SEQ ID NOs: 84, 88, and 90; (8) SEQ ID NOs: 85, 88 and 90; (9) SEQ ID NOs:84, 88 and 91; (10) SEQ ID NOs: 85, 88 and 91; (11) SEQ ID NOs: 84, 88, 89 and 91; (12) SEQ ID NOs: 84, 88, 90 and 91; (13) SEQ ID NOs: 85, 88, 89 and 91: and (14) SEQ ID NOs: 85, 88, 90 and 91. In certain embodiments, the polynucleotide is one that encodes a polypeptide having at least about 60%, 65%, 70%, 75%, 80%, 85%, 90%, 91%, 92%, 93%, 94%, 95%, 96%, 97%, 98%, 99%, or 100% sequence identity to SEQ ID NO:52. In some embodiments, the polynucleotide encodes a GH61 endoglucanase polypeptide (e.g., an EG IV polypeptide from a suitable organism, such as, without limitation, T. reesei Eg4).

In some aspects, the disclosure provides an isolated, synthetic, or recombinant polynucleotide encoding a polypeptide having at least about 70%, (e.g., at least about 71%, 72%, 73%, 74%, 75%, 76%, 77%, 78%, 79%, 80%, 81%, 82%, 83%, 84%, 85%, 86%, 87%, 88%, 89%, 90%, 91%, 92%, 93%, 94%, 95%, 96%, 97%, 98%, or 99%, or complete (100%)) identity to a polypeptide of any one of SEQ ID NOs:2, 4, 6, 8, 10, 12, 14, 16, 18, 20, 22, 24, 26, 28, 30, 32, 34, 36, 38, 40, 42, 43, and 45, over a region of at least about 10, e.g., at least about 15, 20, 25, 30, 35, 40, 45, 50, 55, 60, 65, 70, 75, 80, 85, 90, 95, 100, 125, 150, 175, 200, 225, 250, 275, 300, 325, or 350 residues, or over the full length immature polypeptide, mature polypeptide, catalytic domain (CD) or carbohydrate binding domain (CBM). In some aspects, the disclosure provides an isolated, synthetic, or recombinant polynucleotide having at least about 70% (e.g., at least about 71%, 72%, 73%, 74%, 75%, 76%, 77%, 78%, 79%, 80%, 81%, 82%, 83%, 84%, 85%, 86%, 87%, 88%, 89%, 90%, 91%, 92%, 93%, 94%, 95%, 96%, 97%, 98%, or 99%, or complete (100%)) identity to any one of SEQ ID NOs: 1, 3, 5, 7, 9, 11, 13, 15, 17, 19, 21, 23, 25, 27, 29, 31, 33, 35, 37, 39, and 41, or to a fragment thereof of at least about 10, 20, 30, 40, 50, 60, 70, 80, 90, 100 residues in length. In some embodiments, the disclosure provides an isolated, synthetic, or recombinant polynucleotide that hybridizes under low stringency conditions, medium stringency conditions, high stringency conditions, or very high stringency conditions to any one of SEQ ID NOs: 1, 3, 5, 7, 9, 11, 13, 15, 17, 19, 21, 23, 25, 27, 29, 31, 33, 35, 37, 39, and 41, or to a fragment or subsequence thereof.

Any of the amino acid sequences described herein can be produced together or in conjunction with at least 1, e.g., at least 2, 3, 5, 10, or 20 heterologous amino acids flanking each of the C- and/or N-terminal ends of the specified amino acid sequence, and or deletions of at least 1, e.g., at least 2, 3, 5, 10, or 20 amino acids from the C- and/or N-terminal ends of an enzyme of the disclosure.

Other variations also are within the scope of this disclosure. For example, one or more amino acid residues can be modified to increase or decrease the pl of an enzyme. The change of pl value can be achieved by removing a glutamate residue or substituting it with another amino acid residue.

The disclosure specifically provides β-glucosidase polypeptides, including, e.g., Fv3C, Pa3D, Fv3G, Fv3D, Tr3A (or T. reesei Bgl1),Tr3B (or T. reesei Bgl3), Te3A, An3A, Fo3A, Gz3A, Nh3A, Vd3A, Pa3G, and Tn3B polypeptides. In some embodiments, the β-glucosidase polypetpides is a fusion/chimera β-glucosidase comprises 2 or more β-glucosidase sequences derived from any one of the above-mentioned β-glucosidase polypetpides (including variants or mutants thereof). For example, the β-glucosidase polypeptide is a chimeric/fusion polypeptide comprising a part of Fv3C operably linked to a part of Tr3B. For example, the β-glucosidase polypeptide is a chimeric/fusion polypeptide comprising a first part comprising a contiguous stretch of at least about 200 residues taken from an N-terminal sequence of Fv3C, a second part comprising a linker domain comprising a loop sequence of about 3, 4, 5, 6, 7, 8, 9, 10, or 11 residues in length comprising a sequence derived from Te3A (e.g., comprising an amino acid sequence of FDRRSPG (SEQ ID NO:204), or of FD(R/K)YNIT (SEQ ID NO:205)), and a third part comprising a contiguous stretch of at least about 50 residues derived from a C-terminal sequence of Tr3B.

The disclosure further provides a number of GH61 endoglucanase polypeptides, including, e.g., T. reesei Eg4 (also termed “TrEG4”), T. reesei Eg7 (also termed “TrEG7” or “TrEGb”), TtEG. In certain embodiments, the GH61 endoglucanase polypetpides of the invention is at least 100 residues in length, and comprises comprises one or more of the sequence motifs selected from the group consisting of: (1) SEQ ID NOs:84 and 88; (2) SEQ ID NOs:85 and 88; (3) SEQ ID NO:86; (4) SEQ ID NO:87; (5) SEQ ID NOs:84, 88 and 89; (6) SEQ ID NOs:85, 88, and 89; (7) SEQ ID NOs: 84, 88, and 90; (8) SEQ ID NOs: 85, 88 and 90; (9) SEQ ID NOs:84, 88 and 91; (10) SEQ ID NOs: 85, 88 and 91; (11) SEQ ID NOs: 84, 88, 89 and 91; (12) SEQ ID NOs: 84, 88, 90 and 91; (13) SEQ ID NOs: 85, 88, 89 and 91: and (14) SEQ ID NOs: 85, 88, 90 and 91.

The disclosure further provides various cellulase polypeptides and hemicellulase polypeptides including, e.g., Fv3A, Pf43A, Fv43E, Fv39A, Fv43A, Fv43B, Pa51A, Gz43A, Fo43A, Af43A, Pf51A, AfuXyn2, AfuXyn5, Fv43D, Pf43B, Fv43B, Fv51A, T. reesei Xyn3, T. reesei Xyn2, and T. reesei Bxl1.

A combination of one or more (e.g., 2 or more, 3 or more, 4 or more, 5 or more, or even 6 or more) of these enzymes is suitably present in the engineered enzyme composition of the invention, wherein at least 2 of the enzymes are derived from different biological sources. At least one or more of the enzymes in an engineered enzyme composition of the invention is suitably present in a weight percent that is different from its weight percent in a naturally-occurring composition, relative to the combined weight of proteins in the composition, e.g, at least one of the enzymes can be overexpressed or underexpressed.

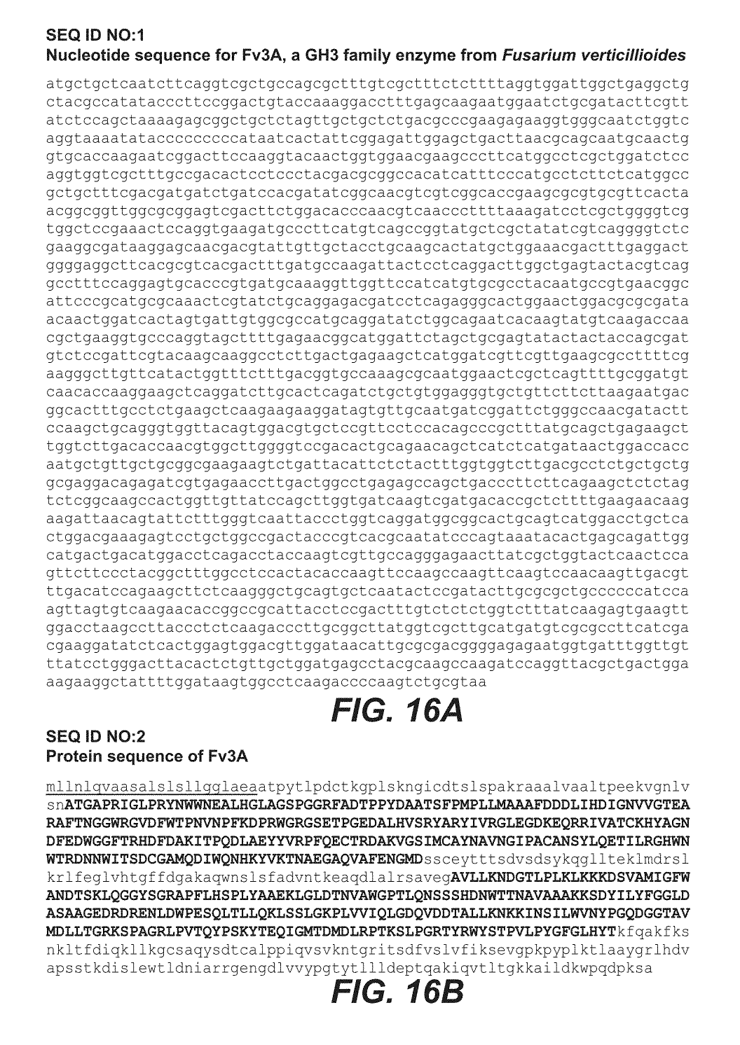

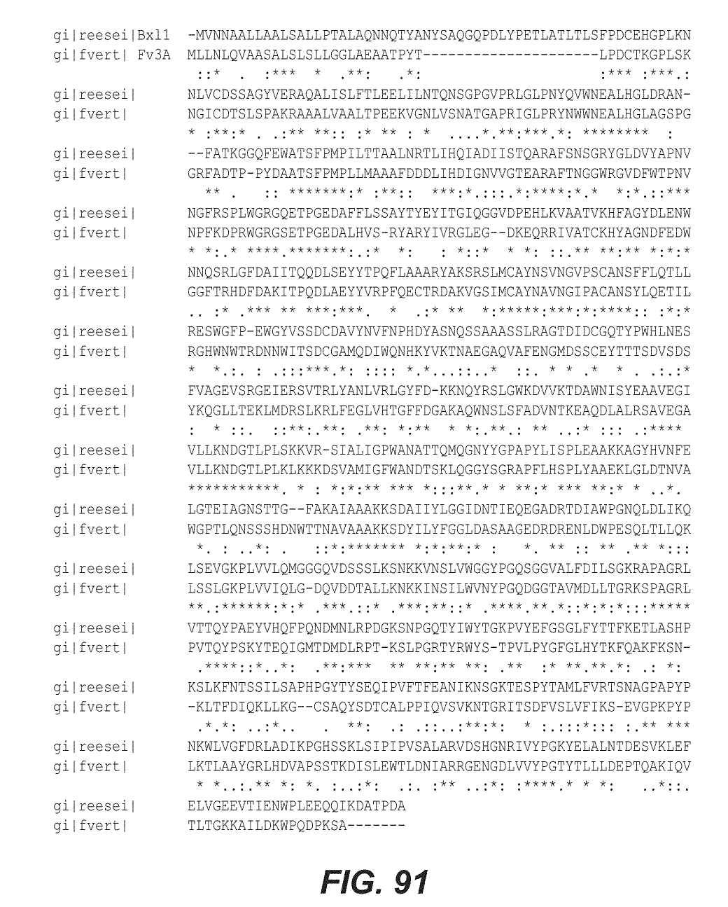

Fv3A: The amino acid sequence of Fv3A (SEQ ID NO:2) is shown in FIGS. 16B and 91. SEQ ID NO:2 is the sequence of the immature Fv3A. Fv3A has a predicted signal sequence corresponding to residues 1 to 23 of SEQ ID NO:2; cleavage of the signal sequence is predicted to yield a mature protein having a sequence corresponding to residues 24 to 766 of SEQ ID NO:2. The predicted conserved domains are in boldface type in FIG. 16B. Fv3A was shown to have β-xylosidase activity, e.g., in an enzymatic assay using p-nitophenyl-β-xylopyranoside, xylobiose, mixed linear xylo-oligomers, branched arabinoxylan oligomers from hemicellulose, or dilute ammonia pretreated corncob as substrates. The predicted catalytic residue is D291, while the flanking residues, S290 and C292, are predicted to be involved in substrate binding. E175 and E213 are conserved across other GH3 and GH39 enzymes and are predicted to have catalytic functions. As used herein, “an Fv3A polypeptide” refers to a polypeptide and/or to a variant thereof comprising a sequence having at least 85%, e.g., at least 86%, 87%, 88%, 89%, 90%, 91%, 92%, 93%, 94%, 95%, 96%, 97%, 98%, 99%, or 100% sequence identity to at least 50, e.g., at least 75, 100, 125, 150, 175, 200, 250, 300, 350, 400, 450, 500, 550, 600, 650, or 700 contiguous amino acid residues among residues 24 to 766 of SEQ ID NO:2. An Fv3A polypeptide preferably is unaltered as compared to native Fv3A in residues D291, S290, C292, E175, and E213. An Fv3A polypeptide is preferably unaltered in at least 70%, 75%, 80%, 85%, 90%, 95%, 98%, or 99% of the amino acid residues that are conserved among Fv3A, T. reesei Bxl1 and/or T. reesei Bgl1, as shown in the alignment of FIG. 91. An Fv3A polypeptide suitably comprises the entire predicted conserved domain of native Fv3A as shown in FIG. 16B. The Fv3A polypeptide of the invention has β-xylosidase activity, having at least 90%, 91%, 92%, 93%, 94%, 95%, 96%, 97%, 98%, 99%, or 100% sequence identity to the amino acid sequence of SEQ ID NO:2, or to residues (i) 24-766, (ii) 73-321, (iii) 73-394, (iv) 395-622, (v) 24-622, or (vi) 73-622 of SEQ ID NO:2.

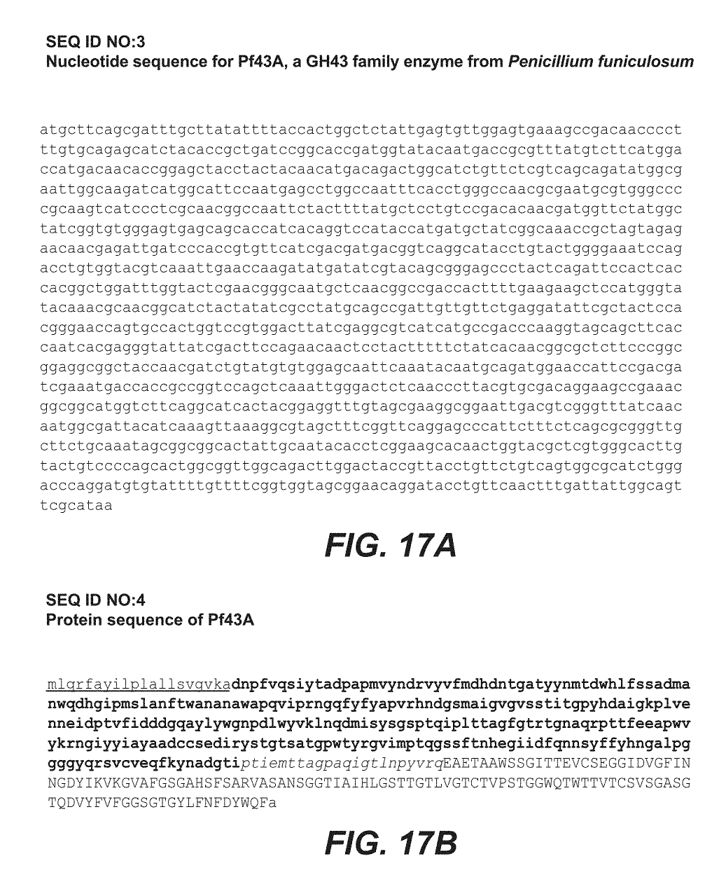

Pf43A: The amino acid sequence of Pf43A (SEQ ID NO:4) is shown in FIGS. 17B and 93. SEQ ID NO:4 is the sequence of the immature Pf43A. Pf43A has a predicted signal sequence corresponding to residues 1 to 20 of SEQ ID NO:4; cleavage of the signal sequence is predicted to yield a mature protein having a sequence corresponding to residues 21 to 445 of SEQ ID NO:4. The predicted conserved domain is in boldface type, the predicted CBM is in uppercase type, and the predicted linker separating the CD and CBM is in italics in FIG. 17B. Pf43A has been shown to have β-xylosidase activity, in, for e.g., an enzymatic assay using p-nitophenyl-β-xylopyranoside, xylobiose, mixed linear xylo-oligomers, or ammonia pretreated corncob as substrates. The predicted catalytic residues include either D32 or D60, D145, and E206. The C-terminal region underlined in FIG. 93 is the predicted CBM. As used herein, “a Pf43A polypeptide” refers to a polypeptide and/or a variant thereof comprising a sequence having at least 85%, 86%, 87%, 88%, 89%, 90%, 91%, 92%, 93%, 94%, 95%, 96%, 97%, 98%, 99%, or 100% sequence identity to at least 50, 75, 100, 125, 150, 175, 200, 250, 300, 350, or 400 contiguous amino acid residues among residues 21 to 445 of SEQ ID NO:4. A Pf43A polypeptide preferably is unaltered as compared to the native Pf43A in residues D32 or D60, D145, and E206. A Pf43A is preferably unaltered in at least 70%, 80%, 90%, 95%, 98%, or 99% of the amino acid residues that are found conserved across a family of proteins including Pf43A and 1, 2, 3, 4, 5, 6, 7, or all 8 of other amino acid sequences in the alignment of FIG. 93. A Pf43A polypeptide of the invention suitably comprises two or more or all of the following domains: (1) the predicted CBM, (2) the predicted conserved domain, and (3) the linker of Pf43A as shown in FIG. 17B. The Pf43A polypeptide of the invention has β-xylosidase activity, having at least 90%, 91%, 92%, 93%, 94%, 95%, 96%, 97%, 98%, 99%, or 100% sequence identity to the amino acid sequence of SEQ ID NO:4, or to residues (i) 21-445, (ii) 21-301, (iii) 21-323, (iv) 21-444, (v) 302-444, (vi) 302-445, (vii) 324-444, or (viii) 324-445 of SEQ ID NO:4. The polypeptide suitably has β-xylosidase activity.

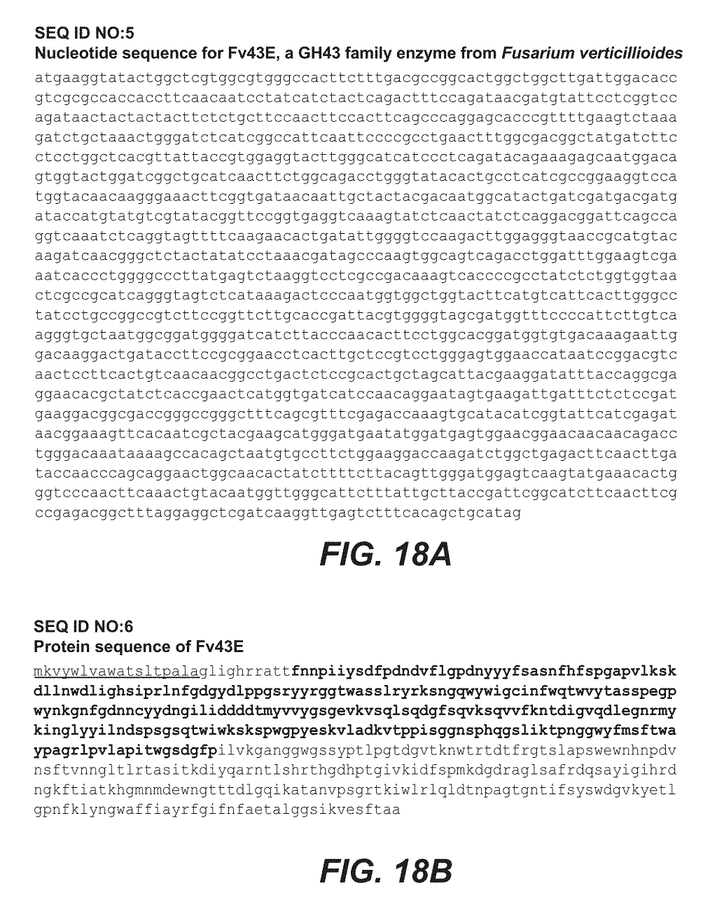

Fv43E: The amino acid sequence of Fv43E (SEQ ID NO:6) is shown in FIGS. 18B and 93. SEQ ID NO:6 is the sequence of the immature Fv43E. Fv43E has a predicted signal sequence corresponding to residues 1 to 18 of SEQ ID NO:6; cleavage of the signal sequence is predicted to yield a mature protein having a sequence corresponding to residues 19 to 530 of SEQ ID NO:6. The predicted conserved domain is marked in boldface type in FIG. 18B. Fv43E was shown to have β-xylosidase activity, in, e.g., enzymatic assay using 4-nitophenyl-β-D-xylopyranoside, xylobiose, and mixed, linear xylo-oligomers, or ammonia pretreated corncob as substrates. The predicted catalytic residues include either D40 or D71, D155, and E241. As used herein, “an Fv43E polypeptide” refers to a polypeptide and/or a variant thereof comprising a sequence having at least 85%, 86%, 87%, 88%, 89%, 90%, 91%, 92%, 93%, 94%, 95%, 96%, 97%, 98%, 99%, or 100% sequence identity to at least 50, 75, 100, 125, 150, 175, 200, 250, 300, 350, 400, 450, or 500 contiguous amino acid residues among residues 19 to 530 of SEQ ID NO:6. An Fv43E polypeptide preferably is unaltered as compared to the native Fv43E in residues D40 or D71, D155, and E241. An Fv43E polypeptide is preferably unaltered in at least 70%, 80%, 90%, 95%, 98%, or 99% of the amino acid residues that are found to be conserved among a family of enzymes including Fv43E, and 1, 2, 3, 4, 5, 6, 7, or all other 8 amino acid sequences in the alignment of FIG. 93. The Fv43E polypeptide of the invention preferably has β-xylosidase activity, having at least 90%, 91%, 92%, 93%, 94%, 95%, 96%, 97%, 98%, 99% or 100% sequence identity to the amino acid sequence of SEQ ID NO:6, or to residues (i) 19-530, (ii) 29-530, (iii) 19-300, or (iv) 29-300 of SEQ ID NO:6.

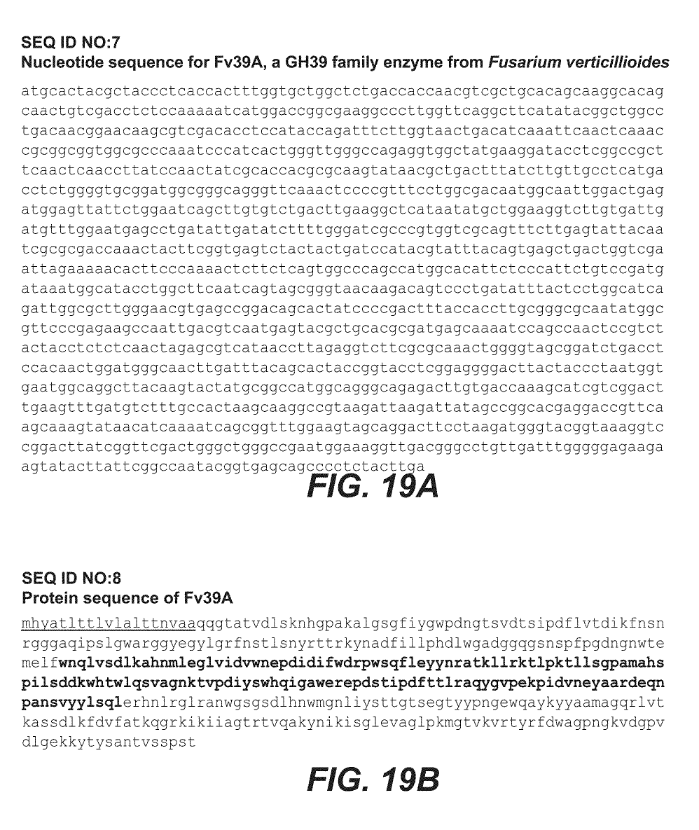

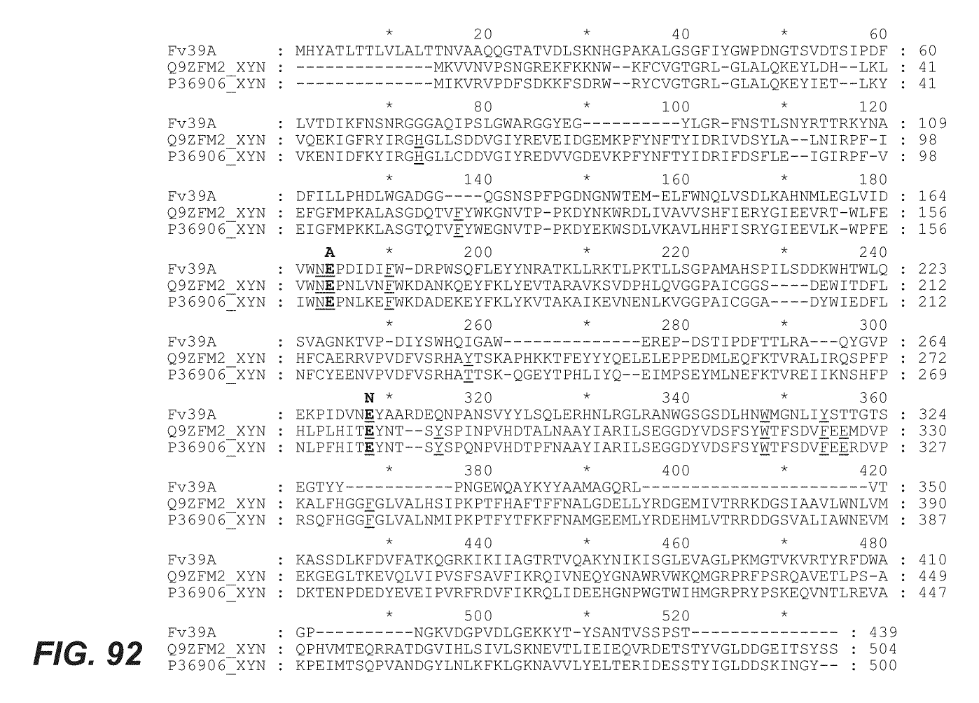

Fv39A: The amino acid sequence of Fv39A (SEQ ID NO:8) is shown in FIGS. 19B and 92. SEQ ID NO:8 is the sequence of the immature Fv39A. Fv39A has a predicted signal sequence corresponding to residues 1 to 19 of SEQ ID NO:8; cleavage of the signal sequence is predicted to yield a mature protein having a sequence corresponding to residues 20 to 439 of SEQ ID NO:8. The predicted conserved domain is shown in boldface type in FIG. 19B. Fv39A was shown to have β-xylosidase activity in, e.g., an enzymatic assay using p-nitophenyl-β-xylopyranoside, xylobiose or mixed, linear xylo-oligomers as substrates. Fv39A residues E168 and E272 are predicted to function as catalytic acid-base and nucleophile, respectively, based on a sequence alignment of the above-mentioned GH39 xylosidases from T. saccharolyticum (Uniprot Accession No. P36906) and G. stearothermophilus (Uniprot Accession No. Q9ZFM2) with Fv39A. As used herein, “an Fv39A polypeptide” refers to a polypeptide and/or a variant thereof comprising a sequence having at least 85%, 86%, 87%, 88%, 89%, 90%, 91%, 92%, 93%, 94%, 95%, 96%, 97%, 98%, 99%, or 100% sequence identity to at least 50, 75, 100, 125, 150, 175, 200, 250, 300, 350, or 400 contiguous amino acid residues among residues 20 to 439 of SEQ ID NO:8. An Fv39A polypeptide preferably is unaltered as compared to native Fv39A in residues E168 and E272. An Fv39A polypeptide is preferably unaltered in at least 70%, 80%, 90%, 95%, 98%, or 99% of the amino acid residues that are conserved among a family or enzymes including Fv39A and xylosidases from T. saccharolyticum and G. stearothermophilus (see above). An Fv39A polypeptide suitably comprises the entire predicted conserved domain of native Fv39A as shown in FIG. 19B. The Fv39A polypeptide of the invention preferably has β-xylosidase activity, having at least 90%, 91%, 92%, 93%, 94%, 95%, 96%, 97%, 98%, 99% or 100% sequence identity to the amino acid sequence of SEQ ID NO:8, or to residues (i) 20-439, (ii) 20-291, (iii) 145-291, or (iv) 145-439 of SEQ ID NO:8.

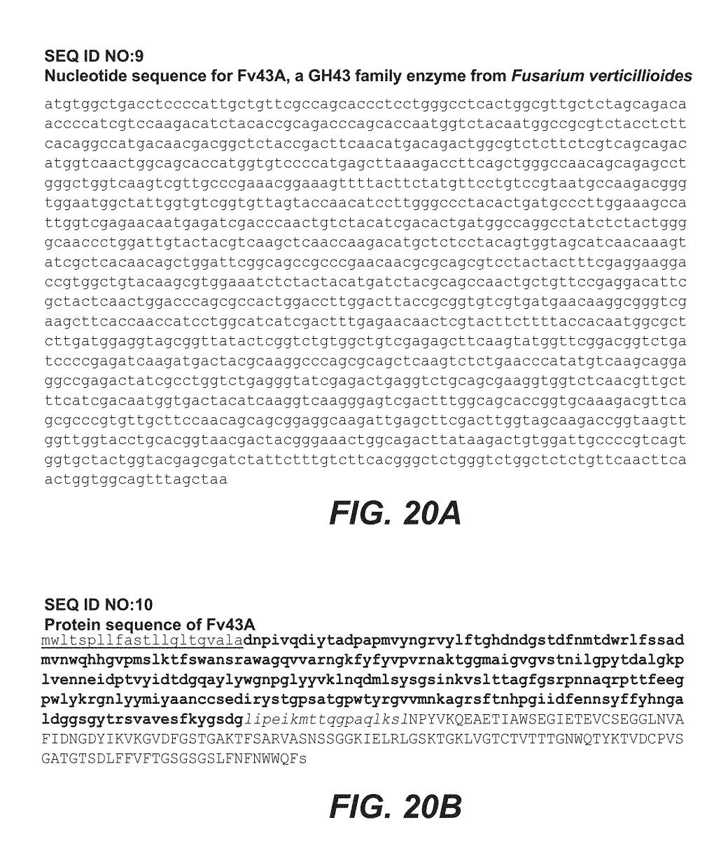

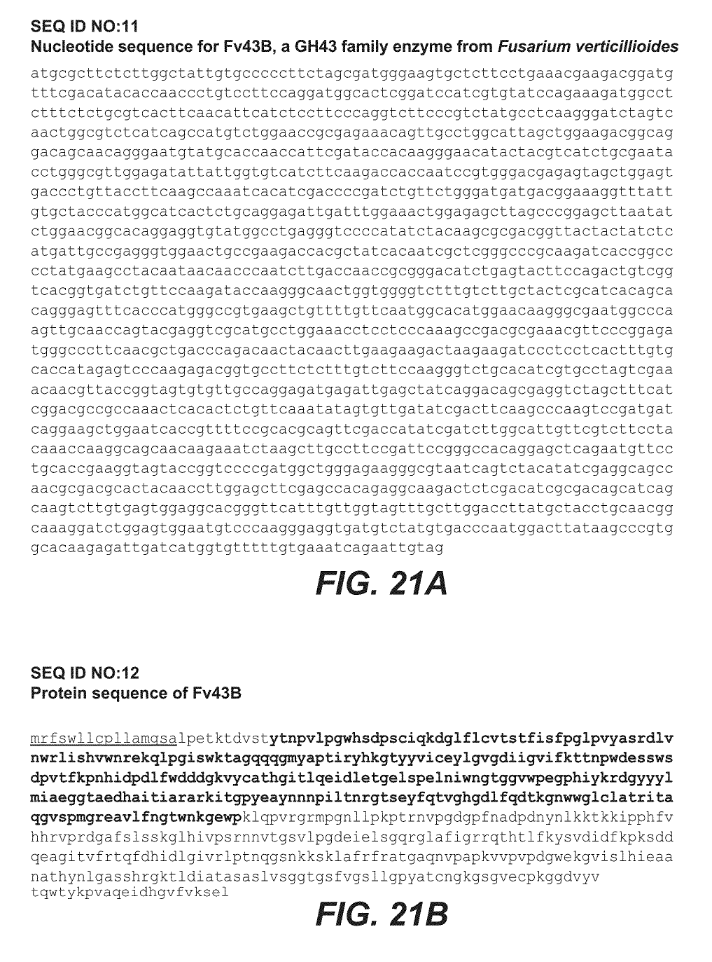

Fv43A: The amino acid sequence of Fv43A (SEQ ID NO:10) is provided in FIGS. 20B and 93. SEQ ID NO:10 is the sequence of the immature Fv43A. Fv43A has a predicted signal sequence corresponding to residues 1 to 22 of SEQ ID NO:10; cleavage of the signal sequence is predicted to yield a mature protein having a sequence corresponding to residues 23 to 449 of SEQ ID NO:10. In FIG. 20B, the predicted conserved domain is in boldface type, the predicted CBM is in uppercase type, and the predicted linker separating the CD and CBM is in italics. Fv43A was shown to have β-xylosidase activity in, e.g., an enzymatic assay using 4-nitophenyl-β-D-xylopyranoside, xylobiose, mixed, linear xylo-oligomers, branched arabinoxylan oligomers from hemicellulose, and/or linear xylo-oligomers as substrates. The predicted catalytic residues including either D34 or D62, D148, and E209. As used herein, “an Fv43A polypeptide” refers to a polypeptide and/or a variant thereof comprising a sequence having at least 85%, 86%, 87%, 88%, 89%, 90%, 91%, 92%, 93%, 94%, 95%, 96%, 97%, 98%, 99%, or 100% sequence identity to at least 50, 75, 100, 125, 150, 175, 200, 250, 300, 350, or 400 contiguous amino acid residues among residues 23 to 449 of SEQ ID NO:10. An Fv43A polypeptide preferably is unaltered, as compared to native Fv43A, at residues D34 or D62, D148, and E209. An Fv43A polypeptide is preferably unaltered in at least 70%, 80%, 90%, 95%, 98%, or 99% of the amino acid residues that are conserved among a family of enzymes including Fv43A and 1, 2, 3, 4, 5, 6, 7, 8, or all 9 other amino acid sequences in the alignment of FIG. 93. An Fv43A polypeptide suitably comprises the entire predicted CBM of native Fv43A, and/or the entire predicted conserved domain of native Fv43A, and/or the linker of Fv43A as shown in FIG. 20B. The Fv45A polypeptide of the invention preferably has β-xylosidase activity, having at least 90%, 91%, 92%, 93%, 94%, 95%, 96%, 97%, 98%, 99% or 100% sequence identity to the amino acid sequence of SEQ ID NO:10, or to residues (i) 23-449, (ii) 23-302, (iii) 23-320, (iv) 23-448, (v) 303-448, (vi) 303-449, (vii) 321-448, or (viii) 321-449 of SEQ ID NO:10. Fv43B: The amino acid sequence of Fv43B (SEQ ID NO:12) is shown in FIGS. 21B and 93. SEQ ID NO:12 is the sequence of the immature Fv43B. Fv43B has a predicted signal sequence corresponding to residues 1 to 16 of SEQ ID NO:12; cleavage of the signal sequence is predicted to yield a mature protein having a sequence corresponding to residues 17 to 574 of SEQ ID NO:12. The predicted conserved domain is in boldface type in FIG. 21B. Fv43B was shown to have both β-xylosidase and L-α-arabinofuranosidase activities, in, e.g., a first enzymatic assay using 4-nitophenyl-β-D-xylopyranoside and p-nitrophenyl-α-L-arabinofuranoside as substrates. It was shown in a second enzymatic assay, to catalyze the release of arabinose from branched arabino-xylooligomers and to catalyze the increased xylose release from oligomer mixtures in the presence of other xylosidase enzymes. The predicted catalytic residues include either D38 or D68, D151, and E236. As used herein, “an Fv43B polypeptide” refers to a polypeptide and/or a variant thereof comprising a sequence having at least 85%, 86%, 87%, 88%, 89%, 90%, 91%, 92%, 93%, 94%, 95%, 96%, 97%, 98%, 99%, or 100% sequence identity to at least 50, 75, 100, 125, 150, 175, 200, 250, 300, 350, 400, 450, 500, or 550 contiguous amino acid residues among residues 17 to 574 of SEQ ID NO:12. An Fv43B polypeptide preferably is unaltered, as compared to native Fv43B, at residues D38 or D68, D151, and E236. An Fv43B polypeptide is preferably unaltered in at least 70%, 80%, 90%, 95%, 98%, or 99% of the amino acid residues that are conserved among a family of enzymes including Fv43B and 1, 2, 3, 4, 5, 6, 7, 8, or all 9 other amino acid sequences in the alignment of FIG. 93. An Fv43B polypeptide suitably comprises the entire predicted conserved domain of native Fv43B as shown in FIGS. 21B and 93. The Fv43B polypeptide of the present invention preferably has β-xylosidase activity, L-α-arabinofuranosidase activity, or both β-xylosidase and L-α-arabinofuranosidase activities, having at least 90%, 91%, 92%, 93%, 94%, 95%, 96%, 97%, 98%, 99% or 100% sequence identity to the amino acid sequence of SEQ ID NO:12, or to residues (i) 17-574, (ii) 27-574, (iii) 17-303, or (iv) 27-303 of SEQ ID NO:12.

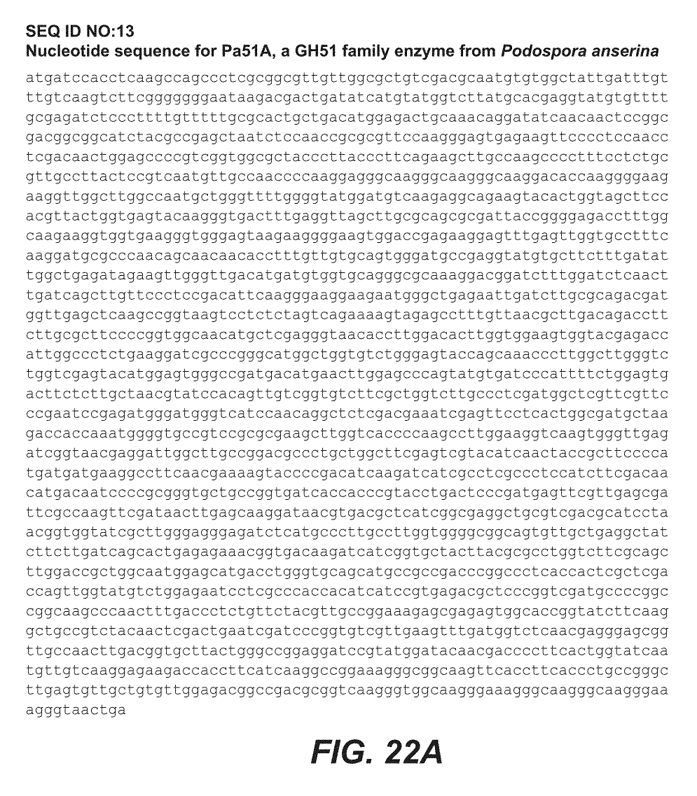

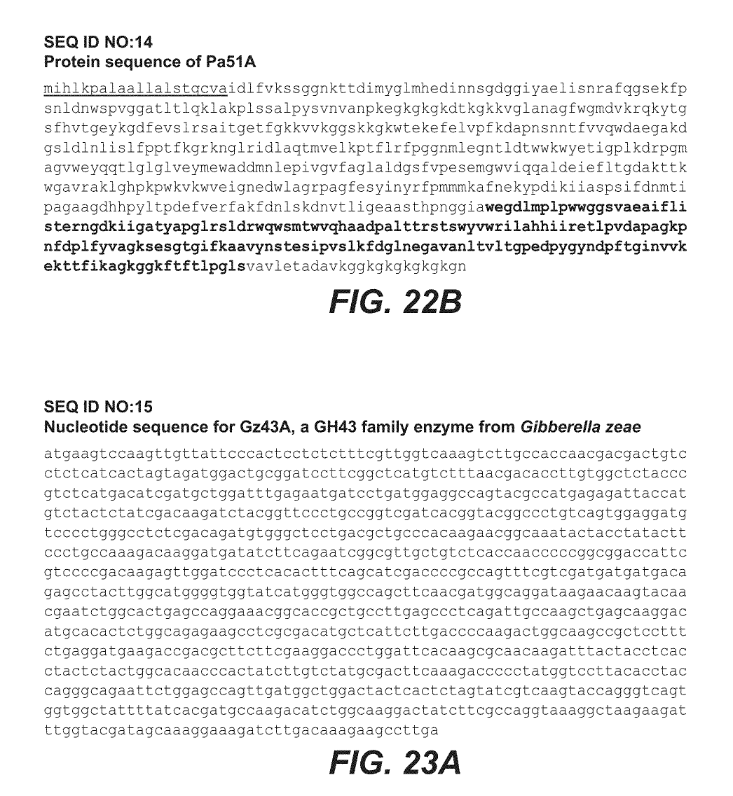

Pa51A: The amino acid sequence of Pa51A (SEQ ID NO:14) is shown in FIGS. 22B and 94. SEQ ID NO:14 is the sequence of the immature Pa51A. Pa51A has a predicted signal sequence corresponding to residues 1 to 20 of SEQ ID NO:14; cleavage of the signal sequence is predicted to yield a mature protein having a sequence corresponding to residues 21 to 676 of SEQ ID NO:14. The predicted L-α-arabinofuranosidase conserved domain is in boldface type in FIG. 22B. Pa51A was shown to have both β-xylosidase activity and L-α-arabinofuranosidase activity in, e.g., enzymatic assays using artificial substrates p-nitrophenyl-β-xylopyranoside and p-nitophenyl- [symbol] -L-arabinofuranoside. It was shown to catalyze the release of arabinose from branched arabino-xylo oligomers and to catalyze the increased xylose release from oligomer mixtures in the presence of other xylosidase enzymes. Conserved acidic residues include E43, D50, E257, E296, E340, E370, E485, and E493. As used herein, “a Pa51A polypeptide” refers to a polypeptide and/or a variant thereof comprising a sequence having at least 85%, 86%, 87%, 88%, 89%, 90%, 91%, 92%, 93%, 94%, 95%, 96%, 97%, 98%, 99%, or 100% sequence identity to at least 50, 75, 100, 125, 150, 175, 200, 250, 300, 350, 400, 450, 500, 550, 600, or 650 contiguous amino acid residues among residues 21 to 676 of SEQ ID NO:14. A Pa51A polypeptide preferably is unaltered, as compared to native Pa51A, at residues E43, D50, E257, E296, E340, E370, E485, and E493. A Pa51A polypeptide is preferably unaltered in at least 70%, 80%, 90%, 95%, 98%, or 99% of the amino acid residues that are conserved among a group of enzymes including Pa51A, Fv51A, and Pf51A, as shown in the alignment of FIG. 94. A Pa51A polypeptide suitably comprises the predicted conserved domain of native Pa51A as shown in FIG. 22B. The Pa51A polypeptide of the invention preferably has β-xylosidase activity, L-α-arabinofuranosidase activity, or both β-xylosidase and L-α-arabinofuranosidase activities, having at least 90%, 91%, 92%, 93%, 94%, 95%, 96%, 97%, 98%, 99% or 100% sequence identity to the amino acid sequence of SEQ ID NO:14, or to residues (i) 21-676, (ii) 21-652, (iii) 469-652, or (iv) 469-676 of SEQ ID NO:14.

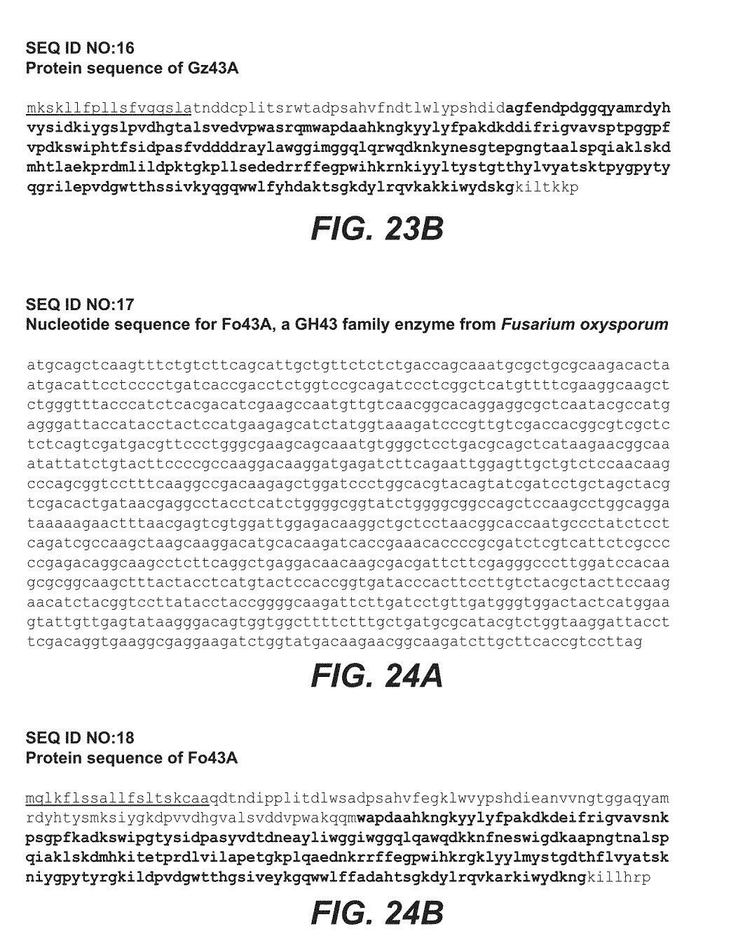

Gz43A: The amino acid sequence of Gz43A (SEQ ID NO:16) is shown in FIGS. 23B and 93. SEQ ID NO:16 is the sequence of the immature Gz43A. Gz43A has a predicted signal sequence corresponding to residues 1 to 18 of SEQ ID NO:16; cleavage of the signal sequence is predicted to yield a mature protein having a sequence corresponding to residues 19 to 340 of SEQ ID NO:16. The predicted conserved domain is in boldface type in FIG. 23B. Gz43A was shown to have β-xylosidase activity in, for example, an enzymatic assay using p-nitophenyl-β-xylopyranoside, xylobiose or mixed, and/or linear xylo-oligomers as substrates. The predicted catalytic residues include either D33 or D68, D154, and E243. As used herein, “a Gz43A polypeptide” refers to a polypeptide and/or a variant thereof comprising a sequence having at least 85%, 86%, 87%, 88%, 89%, 90%, 91%, 92%, 93%, 94%, 95%, 96%, 97%, 98%, 99%, or 100% identity to at least 50, 75, 100, 125, 150, 175, 200, 250, or 300 contiguous amino acid residues among residues 19 to 340 of SEQ ID NO:16. A Gz43A polypeptide preferably is unaltered as compared to native Gz43A at residues D33 or D68, D154, and E243. A Gz43A polypeptide is preferably unaltered in at least 70%, 80%, 90%, 95%, 98%, or 99% of the amino acid residues that are conserved among a group of enzymes including Gz43A and 1, 2, 3, 4, 5, 6, 7, 8 or all 9 other amino acid sequences in the alignment of FIG. 93. A Gz43A polypeptide suitably comprises the predicted conserved domain of native Gz43A shown in FIG. 23B. The Gz43A polypeptide of the invention preferably has β-xylosidase activity having at least 90%, 91%, 92%, 93%, 94%, 95%, 96%, 97%, 98%, 99% or 100% sequence identity to the amino acid sequence of SEQ ID NO:16, or to residues (i) 19-340, (ii) 53-340, (iii) 19-383, or (iv) 53-383 of SEQ ID NO:16.

Fo43A: The amino acid sequence of Fo43A (SEQ ID NO:18) is shown in FIGS. 24B and 93. SEQ ID NO:18 is the sequence of the immature Fo43A. Fo43A has a predicted signal sequence corresponding to residues 1 to 20 of SEQ ID NO:18; cleavage of the signal sequence is predicted to yield a mature protein having a sequence corresponding to residues 21 to 348 of SEQ ID NO:18. The predicted conserved domain is in boldface type in FIG. 24B. Fo43A was shown to have β-xylosidase activity in, e.g., an enzymatic assay using p-nitophenyl-β-xylopyranoside, xylobiose and/or mixed, linear xylo-oligomers as substrates. The predicted catalytic residues include either D37 or D72, D159, and E251. As used herein, “an Fo43A polypeptide” refers to a polypeptide and/or a variant thereof comprising a sequence having at least 85%, 86%, 87%, 88%, 89%, 90%, 91%, 92%, 93%, 94%, 95%, 96%, 97%, 98%, 99%, or 100% sequence identity to at least 50, 75, 100, 125, 150, 175, 200, 250, or 300 contiguous amino acid residues among residues 18 to 344 of SEQ ID NO:18. An Fo43A polypeptide preferably is unaltered, as compared to native Fo43A, at residues D37 or D72, D159, and E251. An Fo43A polypeptide is preferably unaltered in at least 70%, 80%, 90%, 95%, 98%, or 99% of the amino acid residues that are conserved among a group of enzymes including Fo43A and 1, 2, 3, 4, 5, 6, 7, 8 or all 9 other amino acid sequences in the alignment of FIG. 93. The Fo43A polypeptide of the invention preferably has β-xylosidase activity, having at least 90%, 91%, 92%, 93%, 94%, 95%, 96%, 97%, 98%, 99% or 100% sequence identity to the amino acid sequence of SEQ ID NO:18, or to residues (i) 21-341, (ii) 107-341, (iii) 21-348, or (iv) 107-348 of SEQ ID NO:18.

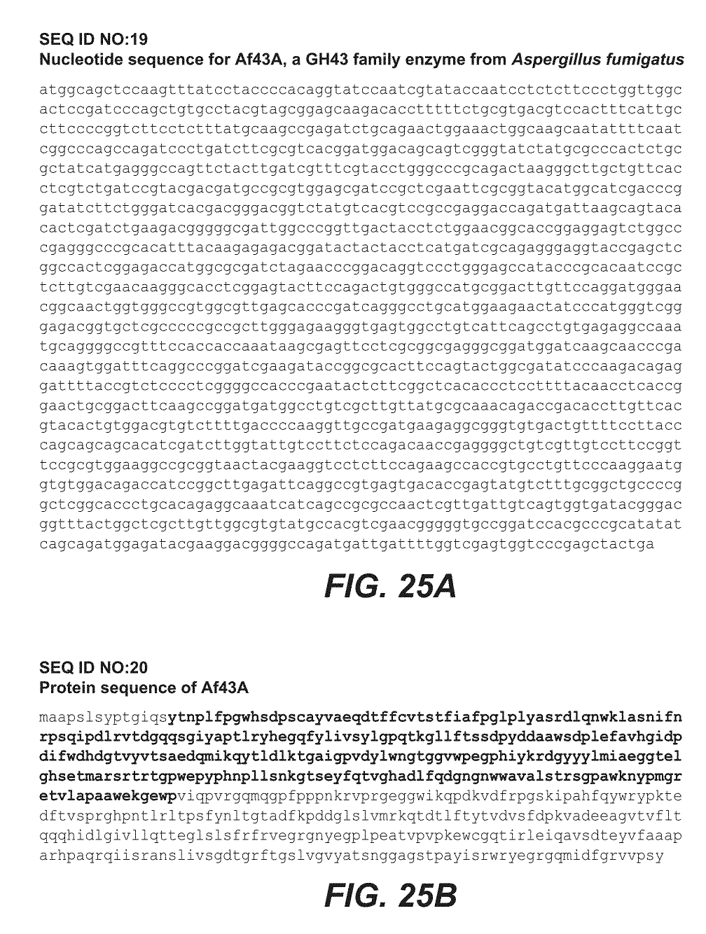

Af43A: The amino acid sequence of Af43A (SEQ ID NO:20) is shown in FIGS. 25B and 93. SEQ ID NO:20 is the sequence of the immature Af43A. The predicted conserved domain is in boldface type in FIG. 25B. Af43A was shown to have L-α-arabinofuranosidase activity in, e.g., an enzymatic assay using p-nitophenyl- [symbol] α-L-arabinofuranoside as a substrate. Af43A was shown to catalyze the release of arabinose from the set of oligomers released from hemicellulose via the action of endoxylanase. The predicted catalytic residues include either D26 or D58, D139, and E227. As used herein, “an Af43A polypeptide” refers to a polypeptide and/or a variant thereof comprising a sequence having at least 85%, 86%, 87%, 88%, 89%, 90%, 91%, 92%, 93%, 94%, 95%, 96%, 97%, 98%, 99%, or 100% sequence identity to at least 50, 75, 100, 125, 150, 175, 200, 250, or 300 contiguous amino acid residues of SEQ ID NO:20. An Af43A polypeptide preferably is unaltered, as compared to native Af43A, at residues D26 or D58, D139, and E227. An Af43A polypeptide is preferably unaltered in at least 70%, 80%, 90%, 95%, 98%, or 99% of the amino acid residues that are conserved among a group of enzymes including Af43A and 1, 2, 3, 4, 5, 6, 7, 8, or all 9 other amino acid sequences in the alignment of FIG. 93. An Af43A polypeptide suitably comprises the predicted conserved domain of native Af43A as shown in FIG. 25B. The Af43A polypeptide of the invention preferably has L-α-arabinofuranosidase activity, having at least 90%, 91%, 92%, 93%, 94%, 95%, 96%, 97%, 98%, 99% or 100% sequence identity to the amino acid sequence of SEQ ID NO:20, or to residues (i)15-558, or (ii)15-295 of SEQ ID NO:20.

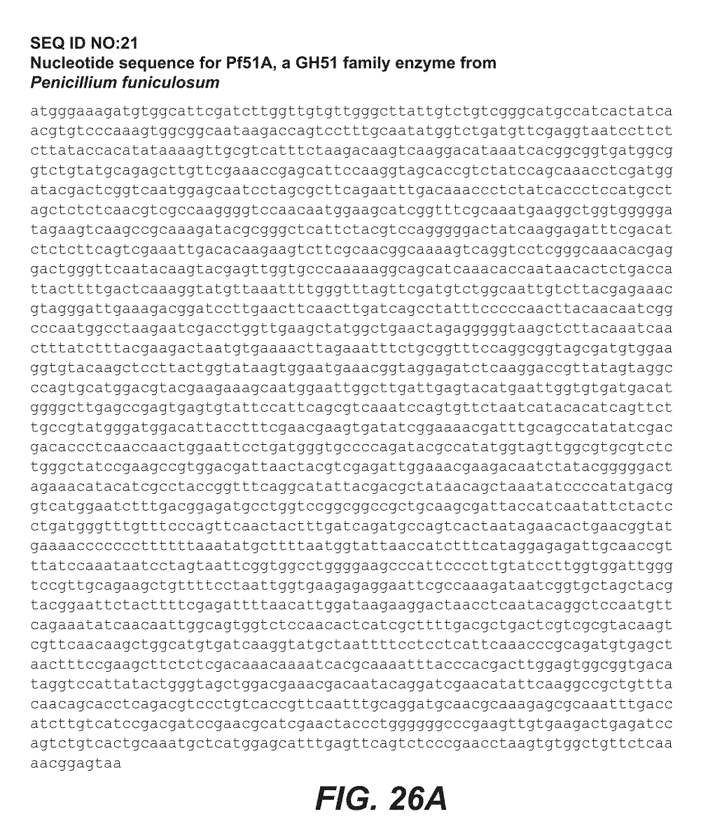

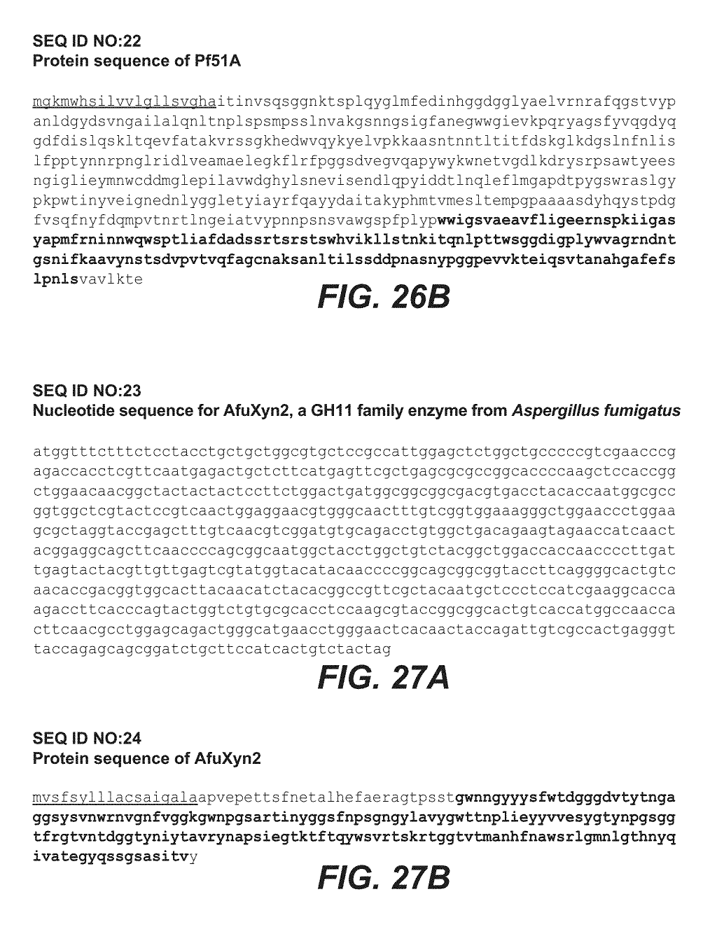

Pf51A: The amino acid sequence of Pf51A (SEQ ID NO:22) is shown in FIGS. 26B and 94. SEQ ID NO:22 is the sequence of the immature Pf51A. Pf51A has a predicted signal sequence corresponding to residues 1 to 20 of SEQ ID NO:22; cleavage of the signal sequence is predicted to yield a mature protein having a sequence corresponding to residues 21 to 642 of SEQ ID NO:22. The predicted L-α-arabinofuranosidase conserved domain is in boldface type in FIG. 26B. Pf51A was shown to have L-α-arabinofuranosidase activity in, for example, an enzymatic assay using 4-nitrophenyl- [symbol] α-L-arabinofuranoside as a substrate. Pf51A was shown to catalyze the release of arabinose from the set of oligomers released from hemicellulose via the action of endoxylanase. The predicted conserved acidic residues include E43, D50, E248, E287, E331, E360, E472, and E480. As used herein, “a Pf51A polypeptide” refers to a polypeptide and/or a variant thereof comprising a sequence having at least 85%, 86%, 87%, 88%, 89%, 90%, 91%, 92%, 93%, 94%, 95%, 96%, 97%, 98%, 99%, or 100% sequence identity to at least 50, 75, 100, 125, 150, 175, 200, 250, 300, 350, 400, 450, 500, 550, or 600 contiguous amino acid residues among residues 21 to 642 of SEQ ID NO:22. A Pf51A polypeptide preferably is unaltered, as compared to native Pf51A, at residues E43, D50, E248, E287, E331, E360, E472, and E480. A Pf51A polypeptide is preferably unaltered in at least 70%, 80%, 90%, 95%, 98%, or 99% of the amino acid residues that are conserved among Pf51A, Pa51A, and Fv51A, as shown in in the alignment of FIG. 94. The Pf51A polypeptide of the invention preferably has L-α-arabinofuranosidase activity, having at least 90%, 91%, 92%, 93%, 94%, 95%, 96%, 97%, 98%, 99% or 100% sequence identity to the amino acid sequence of SEQ ID NO:22, or to residues (i) 21-632, (ii) 461-632, (iii) 21-642, or (iv) 461-642 of SEQ ID NO:22.

AfuXyn2: The amino acid sequence of AfuXyn2 (SEQ ID NO:24) is shown in FIGS. 27B and 95B. SEQ ID NO:24 is the sequence of the immature AfuXyn2. It has a predicted signal sequence corresponding to residues 1 to 18 of SEQ ID NO:24; cleavage of the signal sequence is predicted to yield a mature protein having a sequence corresponding to residues 19 to 228 of SEQ ID NO:24. The predicted GH11 conserved domain is in boldface type in FIG. 27B. AfuXyn2 was shown to have endoxylanase activity indirectly by observing its ability to catalyze the increased xylose monomer production in the presence of xylobiosidase when the enzymes act on pretreated biomass or on isolated hemicellulose.

The conserved catalytic residues include E124, E129, and E215. As used herein, “an AfuXyn2 polypeptide” refers to a polypeptide and/or a variant thereof comprising a sequence having at least 85%, 86%, 87%, 88%, 89%, 90%, 91%, 92%, 93%, 94%, 95%, 96%, 97%, 98%, 99%, or 100% sequence identity to at least 50, 75, 100, 125, 150, 175, or 200 contiguous amino acid residues among residues 19 to 228 of SEQ ID NO:24. An AfuXyn2 polypeptide preferably is unaltered, as compared to native AfuXyn2, at residues E124, E129 and E215. An AfuXyn2 polypeptide is preferably unaltered in at least 70%, 80%, 90%, 95%, 98%, or 99% of the amino acid residues that are conserved among AfuXyn2, AfuXyn5, and T. reesei Xyn2, as shown in the alignment of FIG. 95B. An AfuXyn2 polypeptide suitably comprises the entire predicted conserved domain of native AfuXyn2 shown in FIG. 27B. The AfuXyn2 polypeptide of the invention preferably has xylanase activity.

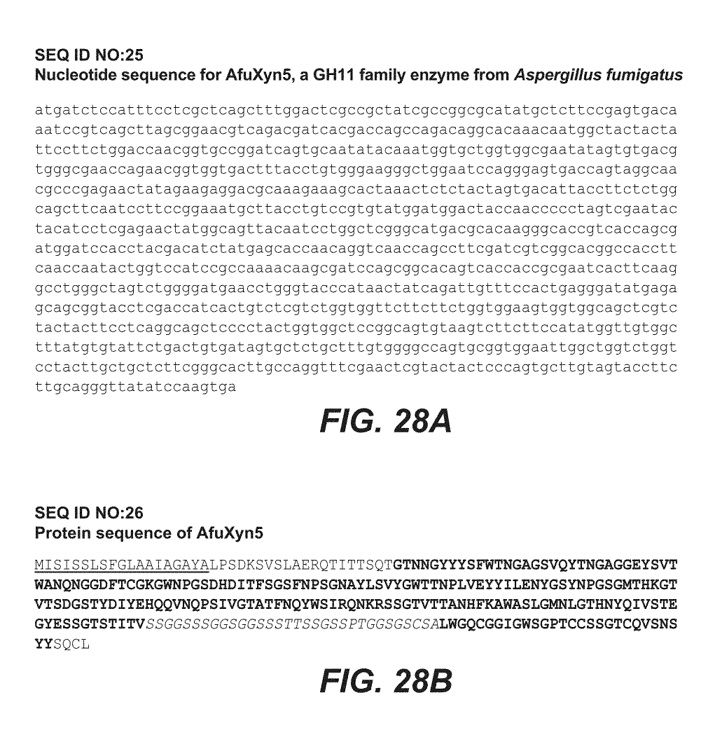

AfuXyn5: The amino acid sequence of AfuXyn5 (SEQ ID NO:26) is shown in FIGS. 28B and 95B. SEQ ID NO:26 is the sequence of the immature AfuXyn5. AfuXyn5 has a predicted signal sequence corresponding to residues 1 to 19 of SEQ ID NO:26 (; cleavage of the signal sequence is predicted to yield a mature protein having a sequence corresponding to residues 20 to 313 of SEQ ID NO:26. The predicted GH11 conserved domains are in boldface type in FIG. 28B. AfuXyn5 was shown to have endoxylanase activity indirectly by observing its ability to catalyze increased xylose monomer production in the presence of xylobiosidase when the enzymes act on pretreated biomass or on isolated hemicellulose.

The conserved catalytic residues include E119, E124, and E210. The predicted CBM is near the C-terminal end, characterized by numerous hydrophobic residues and follows the long serine-, threonine-rich series of amino acids. The region is shown underlined in FIG. 95B. As used herein, “an AfuXyn5 polypeptide” refers to a polypeptide and/or a variant thereof comprising a sequence having at least 85%, 86%, 87%, 88%, 89%, 90%, 91%, 92%, 93%, 94%, 95%, 96%, 97%, 98%, 99%, or 100% sequence identity to at least 50, 75, 100, 125, 150, 175, 200, 250, or 275 contiguous amino acid residues among residues 20 to 313 of SEQ ID NO:26. An AfuXyn5 polypeptide preferably is unaltered, as compared to native AfuXyn5, at residues E119, E120, and E210. An AfuXyn5 polypeptide is preferably unaltered in at least 70%, 80%, 90%, 95%, 98%, or 99% of the amino acid residues that are conserved among AfuXyn5, AfuXyn2, and T. reesei Xyn2, as shown in the alignment of FIG. 95B. An AfuXyn5 polypeptide suitably comprises the entire predicted CBM of native AfuXyn5 and/or the entire predicted conserved domain of native AfuXyn5 (underlined) shown in FIG. 28B. The AfuXyn5 polypeptide of the invention preferably has xylanase activity.

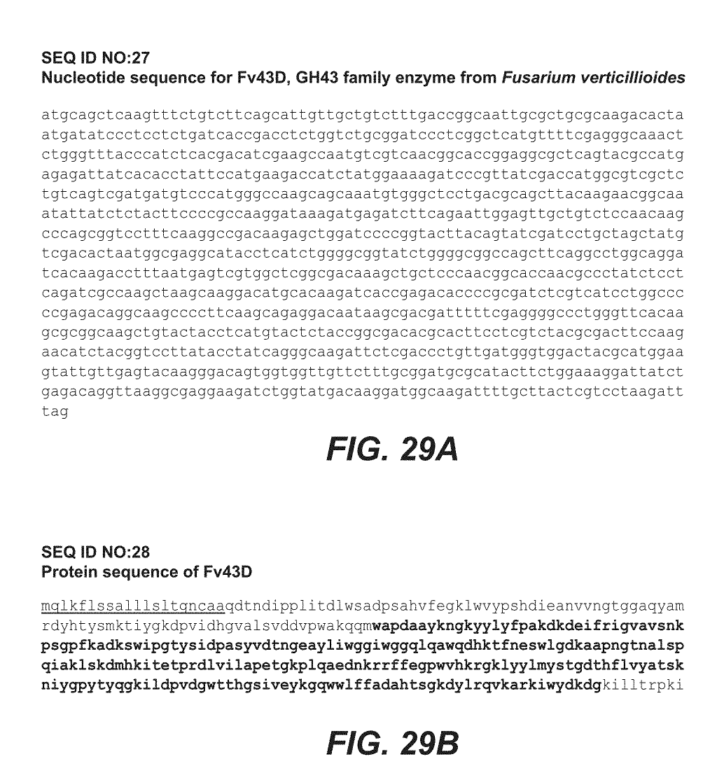

Fv43D: The amino acid sequence of Fv43D (SEQ ID NO:28) is shown in FIGS. 29B and 93. SEQ ID NO:28 is the sequence of the immature Fv43D. Fv43D has a predicted signal sequence corresponding to residues 1 to 20 of SEQ ID NO:28; cleavage of the signal sequence is predicted to yield a mature protein having a sequence corresponding to residues 21 to 350 of SEQ ID NO:28. The predicted conserved domain is in boldface type in FIG. 29B. Fv43D was shown to have β-xylosidase activity in, e.g., an enzymatic assay using p-nitophenyl-β-xylopyranoside, xylobiose, and/or mixed, linear xylo-oligomers as substrates. The predicted catalytic residues include either D37 or D72, D159, and E251. As used herein, “an Fv43D polypeptide” refers to a polypeptide and/or a variant thereof comprising a sequence having at least 85%, 86%, 87%, 88%, 89%, 90%, 91%, 92%, 93%, 94%, 95%, 96%, 97%, 98%, 99%, or 100% sequence identity to at least 50, 75, 100, 125, 150, 175, 200, 250, 300, or 320 contiguous amino acid residues among residues 21 to 350 of SEQ ID NO:28. An Fv43D polypeptide preferably is unaltered, as compared to native Fv43D, at residues D37 or D72, D159, and E251. An Fv43D polypeptide is preferably unaltered in at least 70%, 80%, 90%, 95%, 98%, or 99% of the amino acid residues that are conserved among a group of enzymes including Fv43D and 1, 2, 3, 4, 5, 6, 7, 8, or all 9 other amino acid sequences in the alignment of FIG. 93. An Fv43D polypeptide suitably comprises the entire predicted CD of native Fv43D shown in FIG. 29B. The Fv43D polypeptide of the invention preferably has β-xylosidase activity having at least 90%, 91%, 92%, 93%, 94%, 95%, 96%, 97%, 98%, 99% or 100% sequence identity to the amino acid sequence of SEQ ID NO:28, or to residues (i) 20-341, (ii) 21-350, (iii) 107-341, or (iv) 107-350 of SEQ ID NO:28.

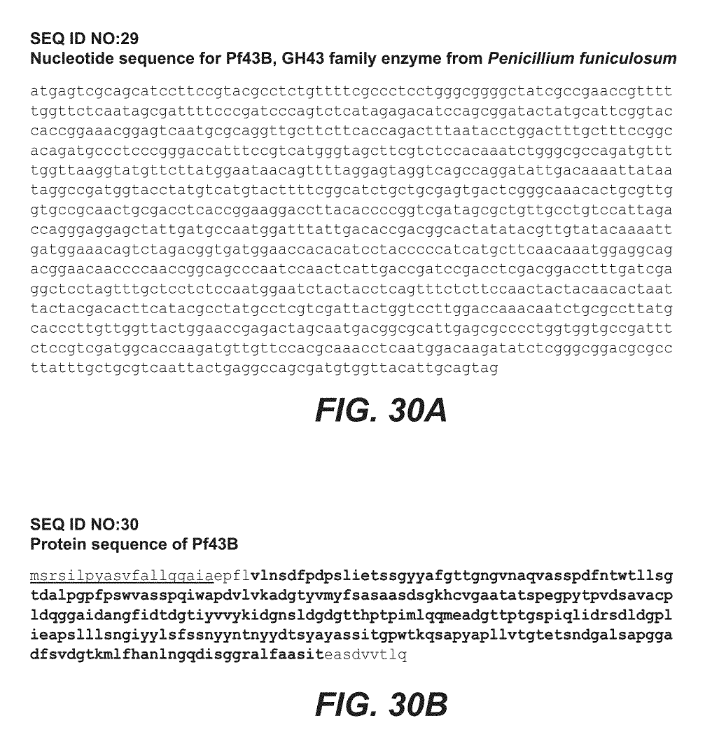

Pf43B: The amino acid sequence of Pf43B (SEQ ID NO:30) is shown in FIGS. 30B and 93. SEQ ID NO:30 is the sequence of the immature Pf43B. Pf43B has a predicted signal sequence corresponding to residues 1 to 20 of SEQ ID NO:30; cleavage of the signal sequence is predicted to yield a mature protein having a sequence corresponding to residues 21 to 321 of SEQ ID NO:30. The predicted conserved domain is in boldface type in FIG. 30B. Conserved acidic residues within the conserved domain include D32, D61, D148, and E212. Pf43B was shown to have β-xylosidase activity in, e.g., an enzymatic assay using p-nitrophenyl-β-xylopyranoside, xylobiose, and/or mixed, linear xylo-oligomers as substrates. As used herein, “a Pf43B polypeptide” refers to a polypeptide and/or a variant thereof comprising a sequence having at least 85%, 86%, 87%, 88%, 89%, 90%, 91%, 92%, 93%, 94%, 95%, 96%, 97%, 98%, 99%, or 100% identity to at least 50, 75, 100, 125, 150, 175, 200, 250, or 280 contiguous amino acid residues among residues 21 to 321 of SEQ ID NO:30. A Pf43B polypeptide preferably is unaltered, as compared to native Pf43B, at residues D32, D61, D148, and E212. A Pf43B polypeptide is preferably unaltered in at least 70%, 80%, 90%, 95%, 98%, or 99% of the amino acid residues that are conserved among a group of enzymes including Pf43B and 1, 2, 3, 4, 5, 6, 7, 8, or all 9 other amino acid sequences in the alignment of FIG. 93. A Pf43B polypeptide suitably comprises the predicted conserved domain of native Pf43B shown in FIG. 30B. The Pf43B polypeptide of the invention preferably has β-xylosidase activity, having at least 90%, 91%, 92%, 93%, 94%, 95%, 96%, 97%, 98%, 99% or 100% sequence identity to the amino acid sequence of SEQ ID NO:30.

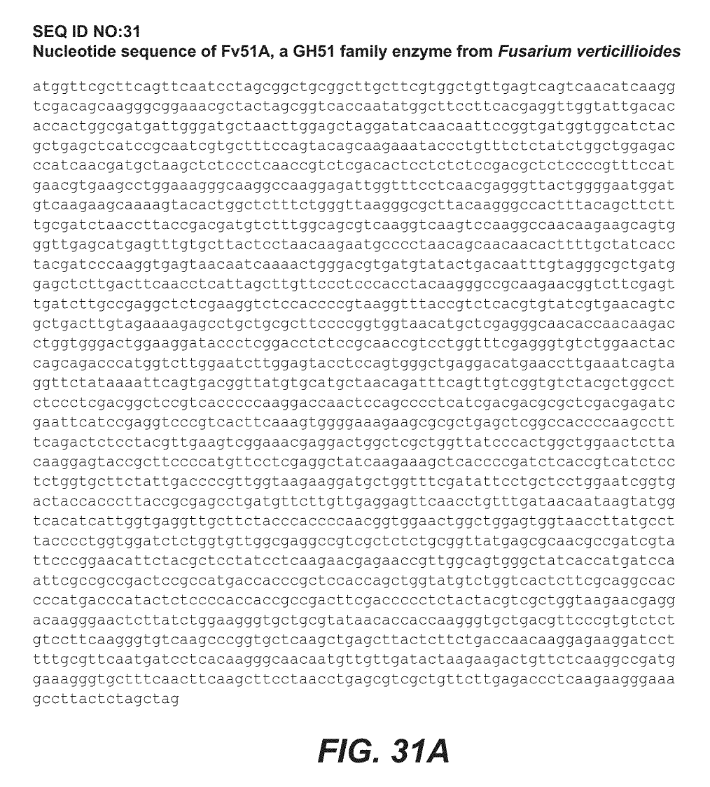

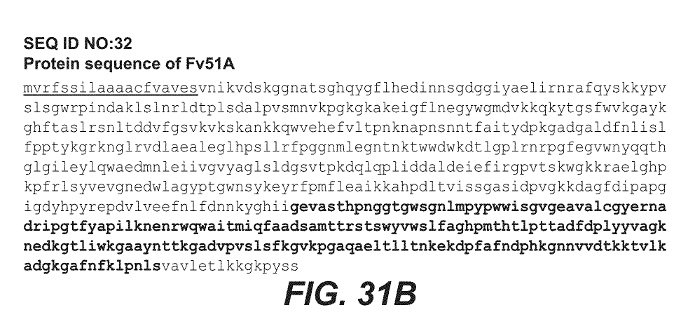

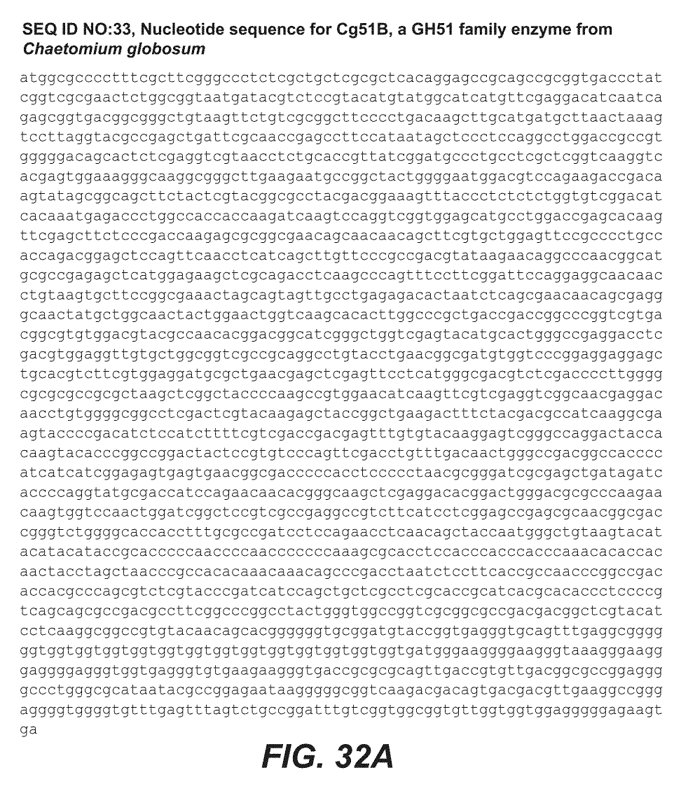

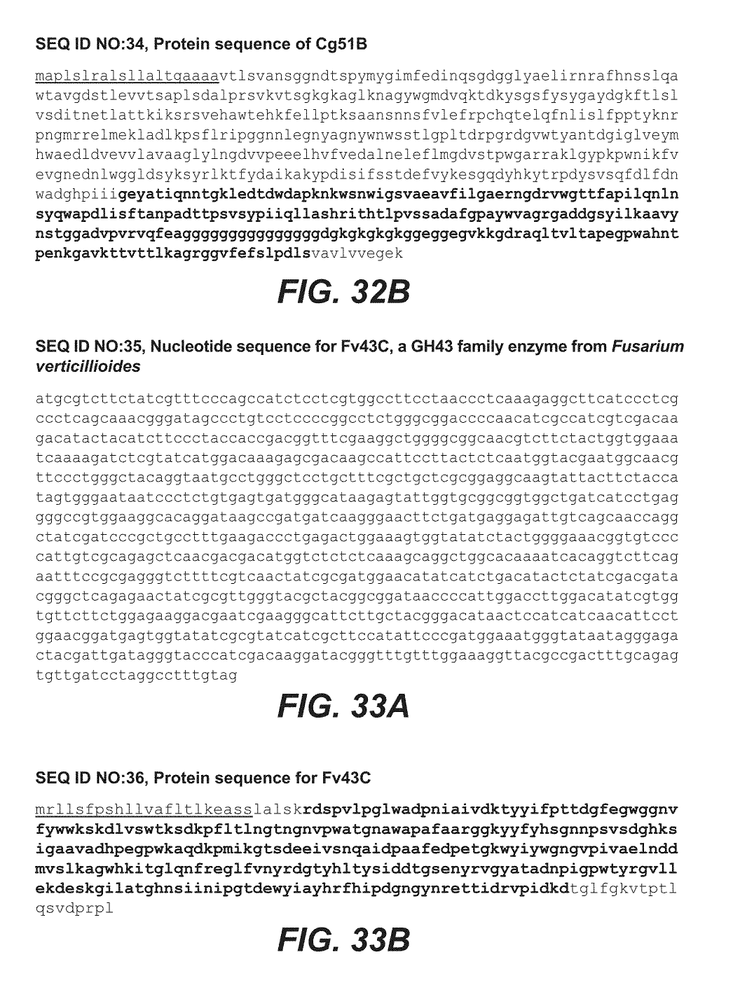

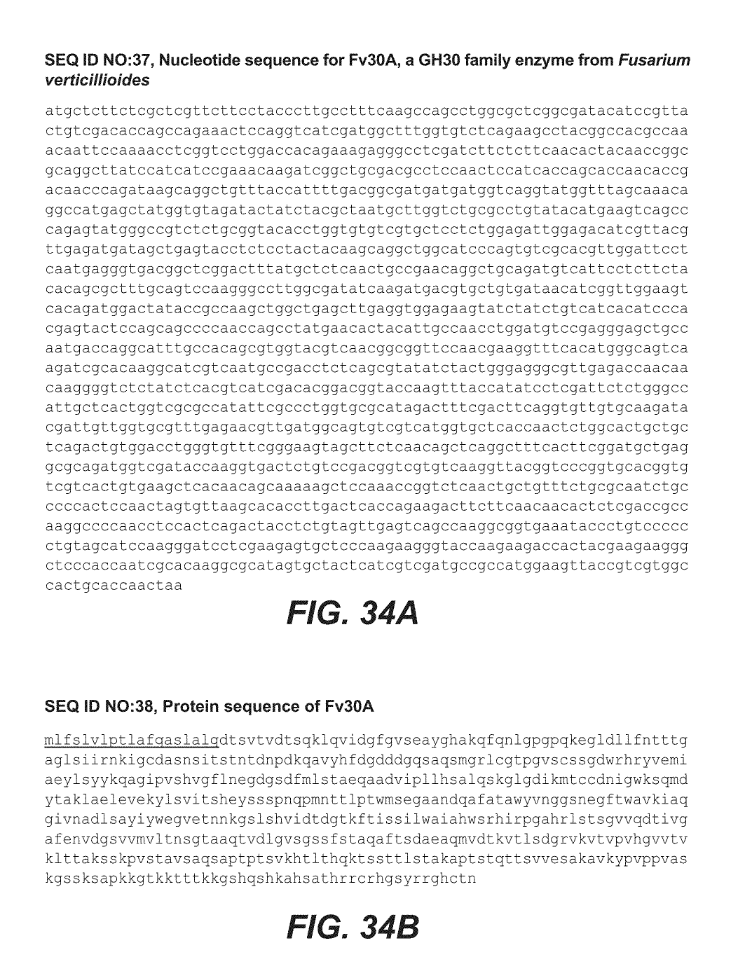

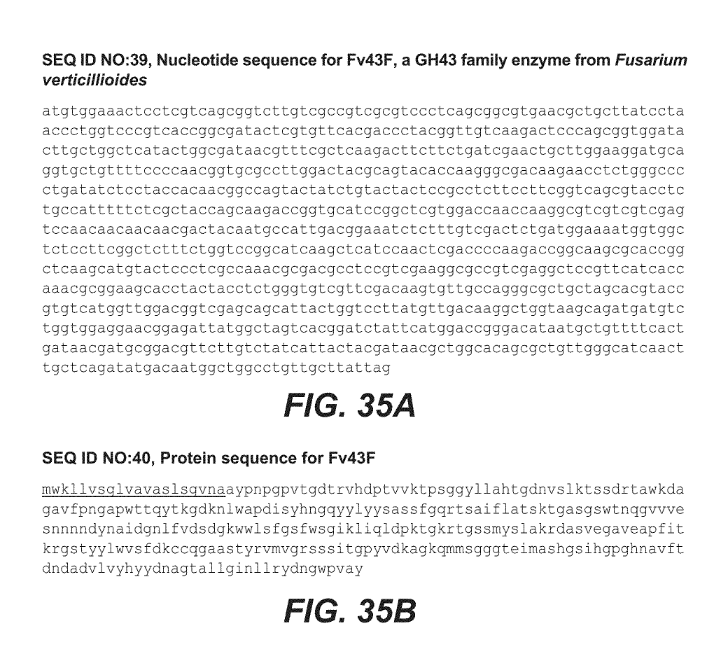

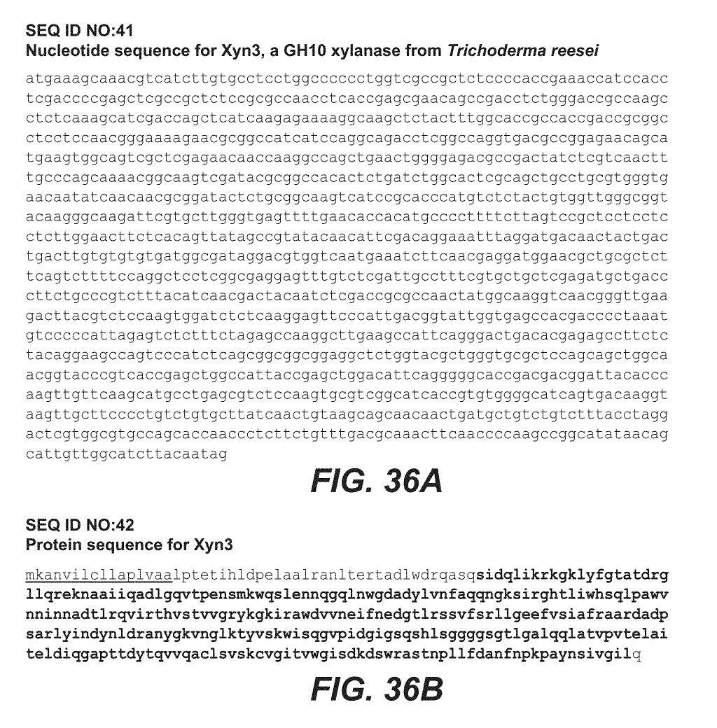

Fv51A: The amino acid sequence of Fv51A (SEQ ID NO:32) is shown in FIGS. 31B and 94. SEQ ID NO:32 is the sequence of the immature Fv51A. Fv51A has a predicted signal sequence corresponding to residues 1 to 19 of SEQ ID NO:32; cleavage of the signal sequence is predicted to yield a mature protein having a sequence corresponding to residues 20 to 660 of SEQ ID NO:32. The predicted L-α-arabinofuranosidase conserved domain is in boldface in FIG. 31B. Fv51A was shown to have L-α-arabinofuranosidase activity in, e.g., an enzymatic assay using 4-nitrophenyl- [symbol] α-L-arabinofuranoside as a substrate. Fv51A was shown to catalyze the release of arabinose from the set of oligomers released from hemicellulose via the action of endoxylanase. Conserved residues include E42, D49, E247, E286, E330, E359, E479, and E487. As used herein, “an Fv51A polypeptide” refers to a polypeptide and/or a variant thereof comprising a sequence having at least 85%, 86%, 87%, 88%, 89%, 90%, 91%, 92%, 93%, 94%, 95%, 96%, 97%, 98%, 99%, or 100% identity to at least 50, 75, 100, 125, 150, 175, 200, 250, 300, 350, 400, 450, 500, 550, 600, or 625 contiguous amino acid residues among residues 20 to 660 of SEQ ID NO:32. An Fv51A polypeptide preferably is unaltered, as compared to native Fv51A, at residues E42, D49, E247, E286, E330, E359, E479, and E487. An Fv51A polypeptide is preferably unaltered in at least 70%, 80%, 90%, 95%, 98%, or 99% of the amino acid residues that are conserved among Fv51A, Pa51A, and Pf51A, as shown in the alignment of FIG. 94. An Fv51A polypeptide suitably comprises the predicted conserved domain of native Fv51A shown in FIG. 31B. The Fv51A polypeptide of the invention preferably has L-α-arabinofuranosidase activity, having at least 90%, 91%, 92%, 93%, 94%, 95%, 96%, 97%, 98%, 99% or 100% sequence identity to the amino acid sequence of SEQ ID NO:32, or to residues (i) 21-660, (ii) 21-645, (iii) 450-645, or (iv) 450-660 of SEQ ID NO:32. Xyn3: The amino acid sequence of T. reesei Xyn3 (SEQ ID NO:42) is shown in FIGS. 36B and 95A. SEQ ID NO:42 is the sequence of the immature T. reesei Xyn3. T. reesei Xyn3 has a predicted signal sequence corresponding to residues 1 to 16 of SEQ ID NO:42; cleavage of the signal sequence is predicted to yield a mature protein having a sequence corresponding to residues 17 to 347 of SEQ ID NO:42. The predicted conserved domain is in boldface type in FIG. 36B. T. reesei Xyn3 was shown to have endoxylanase activity indirectly by oberservation of its ability to catalyze increased xylose monomer production in the presence of xylobiosidase when the enzymes act on pretreated biomass or on isolated hemicellulose. The conserved catalytic residues include E91, E176, E180, E195, and E282, as determined by alignment with another GH10 family enzyme, the Xys1 delta from

Streptomyces halstedii (Canals et al., 2003, Act Crystalogr. D Biol. 59:1447-53), which has 33% sequence identity to T. reesei Xyn3. As used herein, “a T. reesei Xyn3 polypeptide” refers to a polypeptide and/or a variant thereof comprising a sequence having at least 85%, 86%, 87%, 88%, 89%, 90%, 91%, 92%, 93%, 94%, 95%, 96%, 97%, 98%, 99%, or 100% sequence identity to at least 50, 75, 100, 125, 150, 175, 200, 250, or 300 contiguous amino acid residues among residues 17 to 347 of SEQ ID NO:42. A T. reesei Xyn3 polypeptide preferably is unaltered, as compared to native T. reesei Xyn3, at residues E91, E176, E180, E195, and E282. A T. reesei Xyn3 polypeptide is preferably unaltered in at least 70%, 80%, 90%, 95%, 98%, or 99% of the amino acid residues that are conserved between T. reesei Xyn3 and Xys1 delta. A T. reesei Xyn3 polypeptide suitably comprises the entire predicted conserved domain of native T. reesei Xyn3 shown in FIG. 36B. The T. reesei Xyn3 polypetpide of the invention preferably has xylanase activity.

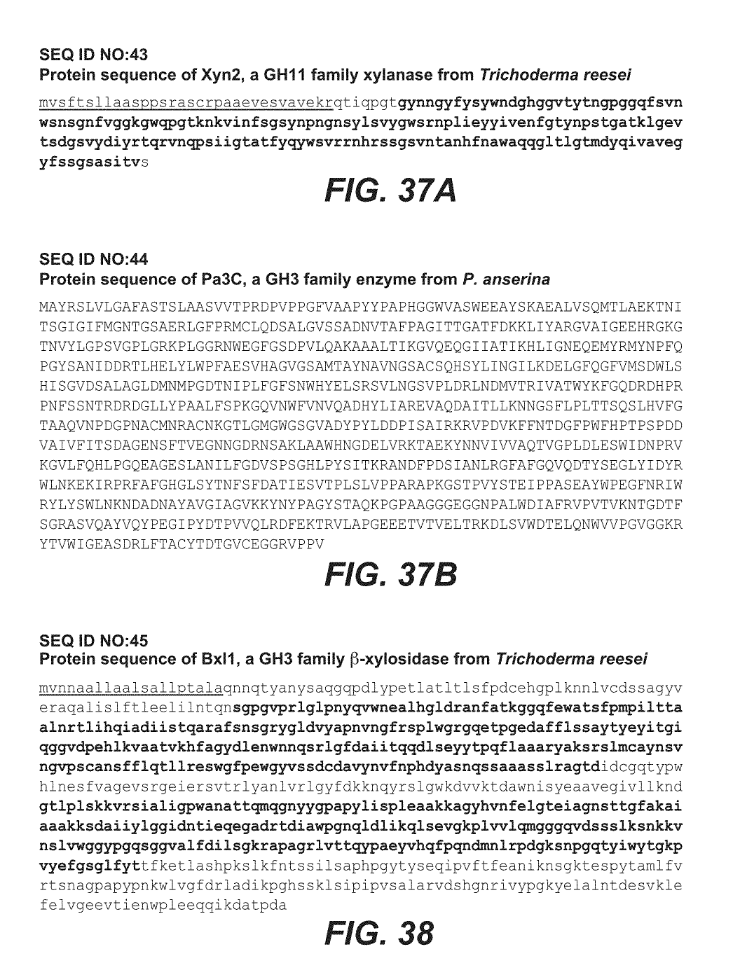

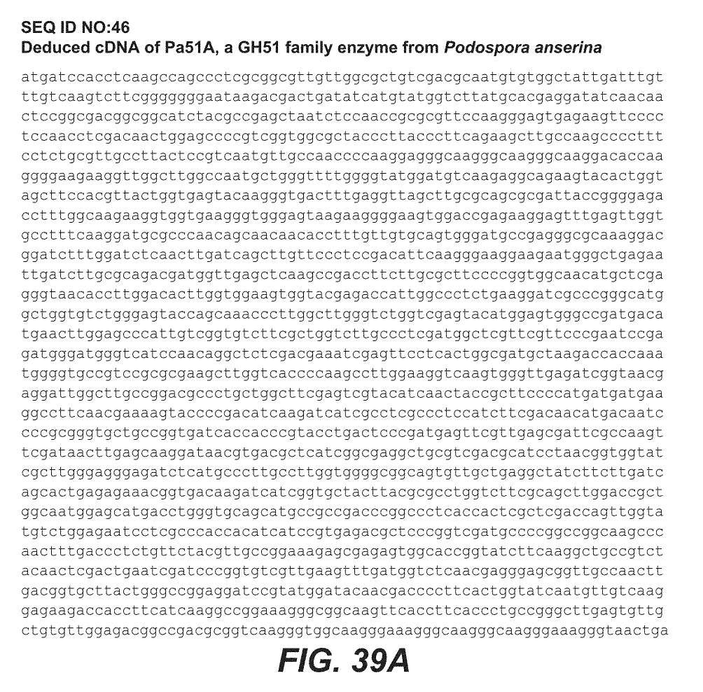

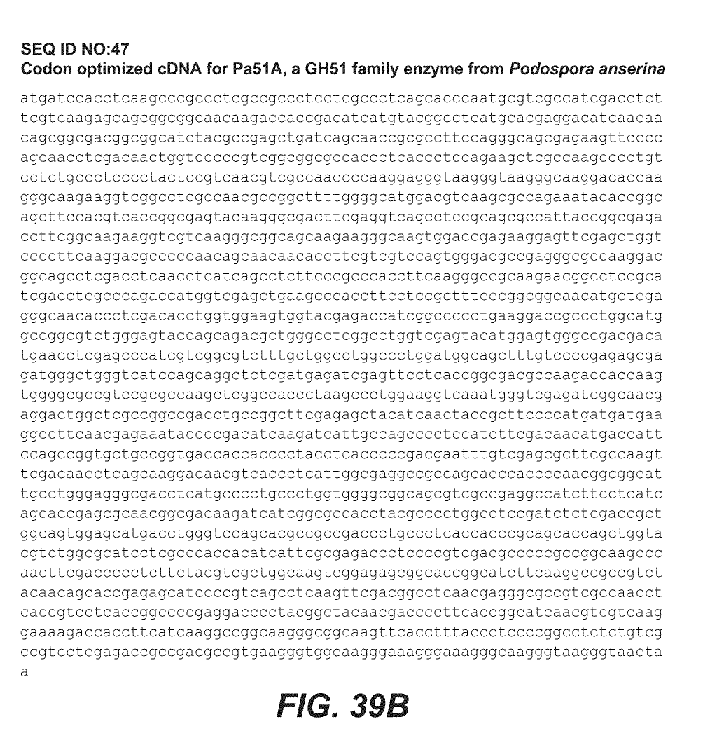

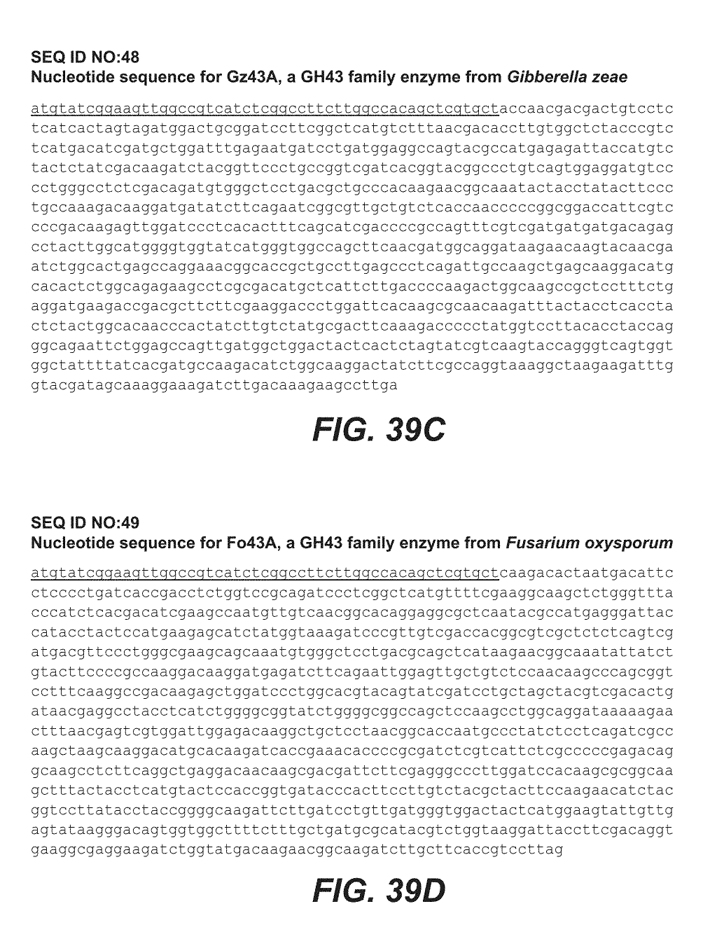

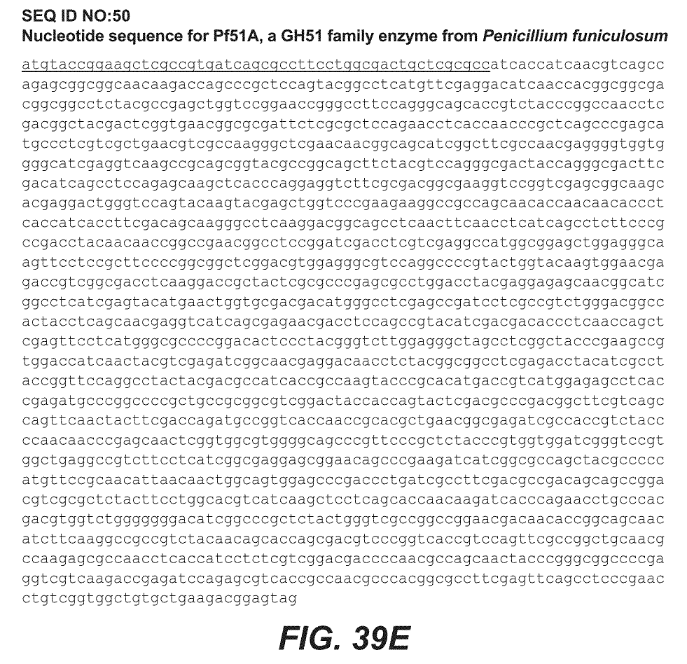

Xyn2: The amino acid sequence of T. reesei Xyn2 (SEQ ID NO:43) is shown in FIGS. 37 and 95B. SEQ ID NO:43 is the sequence of the immature T. reesei Xyn2. T. reesei Xyn2 has a predicted preprppeptide sequence corresponding to residues 1 to 33 of SEQ ID NO:43; cleavage of the predicted signal sequence between positions 16 and 17 is predicted to yield a propeptide, which is processed by a kexin-like protease between positions 32 and 33, generating the mature protein having a sequence corresponding to residues 33 to 222 of SEQ ID NO:43. The predicted conserved domain is in boldface type in FIG. 37. T. reesei Xyn2 was shown to have endoxylanase activity indirectly by observation of its ability to catalyze an increased xylose monomer production in the presence of xylobiosidase when the enzymes act on pretreated biomass or on isolated hemicellulose. The conserved acidic residues include E118, E123, and E209. As used herein, “a T. reesei Xyn2 polypeptide” refers to a polypeptide and/or a variant thereof comprising a sequence having at least 85%, 86%, 87%, 88%, 89%, 90%, 91%, 92%, 93%, 94%, 95%, 96%, 97%, 98%, 99%, or 100% sequence identity to at least 50, 75, 100, 125, 150, or 175 contiguous amino acid residues among residues 33 to 222 of SEQ ID NO:43. A T. reesei Xyn2 polypeptide preferably is unaltered, as compared to a native T. reesei Xyn2, at residues E118, E123, and E209. A T. reesei Xyn2 polypeptide is preferably unaltered in at least 70%, 80%, 90%, 95%, 98%, or 99% of the amino acid residues that are conserved among T. reesei Xyn2, AfuXyn2, and AfuXyn5, as shown in the alignment of FIG. 95B. A T. reesei Xyn2 polypeptide suitably comprises the entire predicted conserved domain of native T. reesei Xyn2 shown in FIG. 37. The T. reesei Xyn2 polypeptide of the invention preferably has xylanase activity.

Bxl1: The amino acid sequence of T. reesei Bxl1 (SEQ ID NO:45) is shown in FIGS. 38 and 91. SEQ ID NO:45 is the sequence of the immature T. reesei Bxl1. T. reesei Bxl1 has a predicted signal sequence corresponding to residues 1 to 18 of SEQ ID NO:45; cleavage of the signal sequence is predicted to yield a mature protein having a sequence corresponding to residues 19 to 797 of SEQ ID NO:45. The predicted conserved domains are in boldface type in FIG. 38. T. reesei Bxl1 was shown to have β-xylosidase activity in, e.g., an enzymatic assay using p-nitophenyl-β-xylopyranoside, xylobiose and/or mixed, linear xylo-oligomers as substrates. The conserved acidic residues include E193, E234, and D310. As used herein, “a T. reesei Bxl1 polypeptide” refers to a polypeptide and/or a variant thereof comprising a sequence having at least 85%, 86%, 87%, 88%, 89%, 90%, 91%, 92%, 93%, 94%, 95%, 96%, 97%, 98%, 99%, or 100% sequence identity to at least 50, 75, 100, 125, 150, 175, 200, 250, 300, 350, 400, 450, 500, 550, 600, 650, 700, or 750 contiguous amino acid residues among residues 17 to 797 of SEQ ID NO:45. A T. reesei Bxl1 polypeptide preferably is unaltered, as compared to a native T. reesei Bxl1, at residues E193, E234, and D310. A T. reesei Bxl1 polypeptide is preferably unaltered in at least 70%, 80%, 90%, 95%, 98%, or 99% of the amino acid residues that are conserved among T. reesei Bxl1, and Fv3A, as shown in the alignment of FIG. 91. A T. reesei Bxl1 polypeptide suitably comprises the entire predicted conserved domains of native T. reesei Bxl1 shown in FIG. 38. The T. reesei Bxl1 polypeptide of the invention preferably has β-xylosidase activity having at least 90%, 91%, 92%, 93%, 94%, 95%, 96%, 97%, 98%, 99% or 100% sequence identity to the amino acid sequence of SEQ ID NO:45.

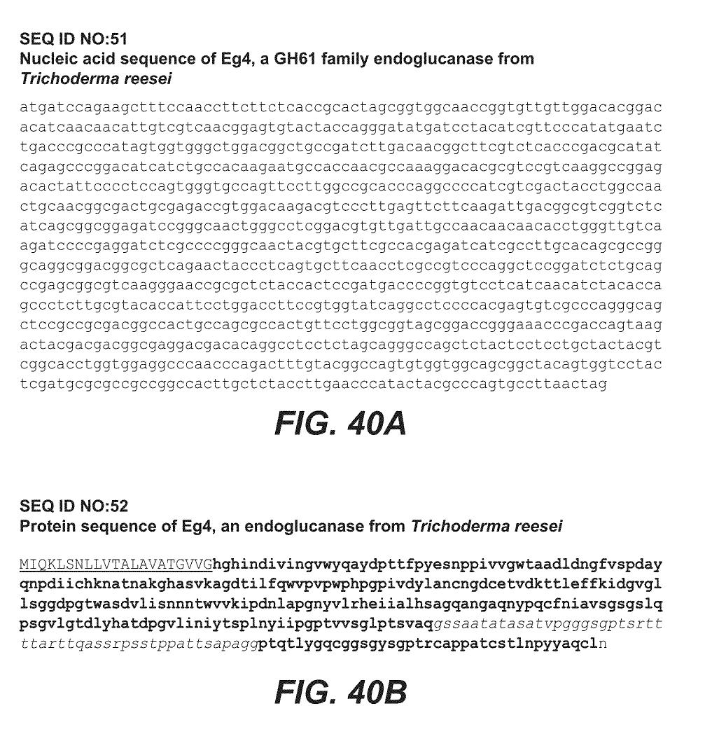

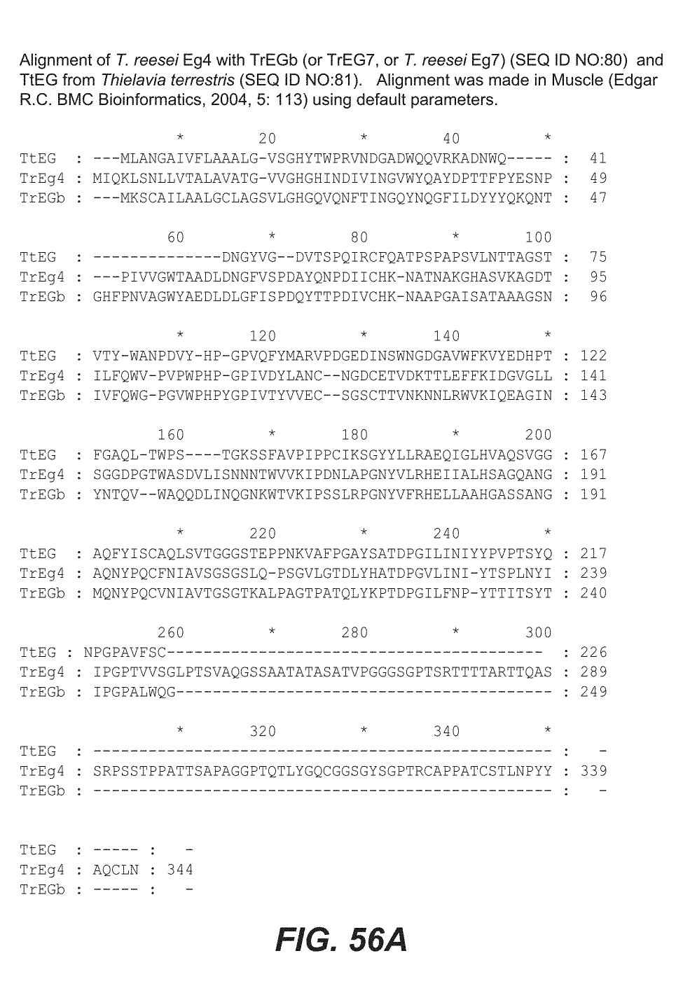

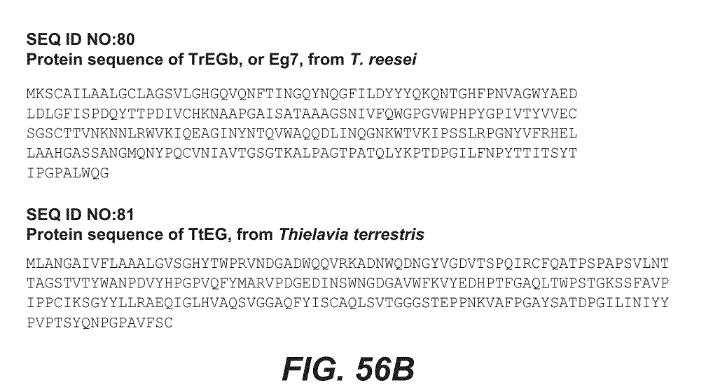

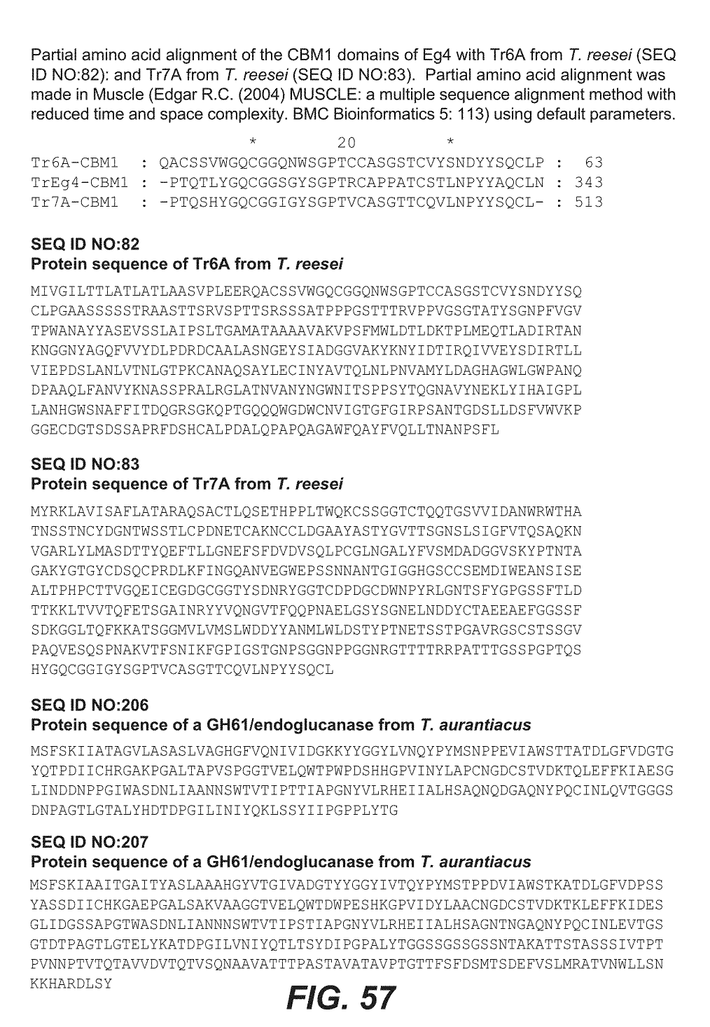

T. reesei Eci4: The amino acid sequence of T. reesei Eg4 (SEQ ID NO:52) is shown in FIGS. 40B and 56. SEQ ID NO:52 is the sequence of the immature T. reesei Eg4. T. reesei Eg4 has a predicted signal sequence corresponding to residues 1 to 21 of SEQ ID NO:52; cleavage of the signal sequence is predicted to yield a mature protein having a sequence corresponding to residues 22 to 344 of SEQ ID NO:52. The predicted conserved domains correspond to residues 22-256 and 307-343 of SEQ ID NO:52, with the latter being the predicted carbohydrate-binding domain (CBM). T. reesei Eg4 was shown to have endoglucanse activity in, e.g., an enzymatic assay using carboxy methyl cellulose as substrates. T. reesei Eg4 residues H22, H107, H184, Q193, Y195 were predicted to function as metal coordinators, residues D61 and G63 were predicted to be conserved surface residues, and residue Y232 were predicted to be involved in activity, based on an amino acid sequence alignment of known endoglucanases, e.g., an endoglucanase from T. terrestris (Accession No. ACE10234, also termed “TtEG” herein), and another endoglucanse Eg7 (Accession No. ADA26043.1) from T. reesei (also termed “TtEG7” or “TrEGb” herein), with T. reesei Eg4 (see, FIG. 56). As used herein, “a T. reesei Eg4 polypeptide” refers to a polypeptide and/or a variant thereof comprising a sequence having at least 85%, 86%, 87%, 88%, 89%, 90%, 91%, 92%, 93%, 94%, 95%, 96%, 97%, 98%, 99%, or 100% identity to at least 50, 75, 100, 125, 150, 175, 200, 250, or 300 contiguous amino acid residues among residues 22 to 344 of SEQ ID NO:52. A T. reesei Eg4 polypeptide preferably is unaltered, as compared to a native T. reesei Eg4, at residues H22, H107, H184, Q193, Y195, D61, G63, and Y232. A T. reesei Eg4 polypeptide is preferably unaltered in at least 70%, 80%, 90%, 95%, 98%, or 99% of the amino acid residues that are conserved among TrEG7, TtEG, and TrEG4, as shown in the alignment of FIG. 56. A T. reesei Eg4 polypeptide suitably comprises the entire predicted conserved domains of native T. reesei Eg4 shown in FIG. 56. The T. reesei Eg4 polypeptide of the invention preferably has endoglucanse IV (EGIV) activity having at least 90%, 91%, 92%, 93%, 94%, 95%, 96%, 97%, 98%, 99% or 100% sequence identity to the amino acid sequence of SEQ ID NO:52, or to residues (i) 22-255, (ii) 22-343, (iii) 307-343, (iv) 307-344, or (v) 22-344 of SEQ ID NO:52.

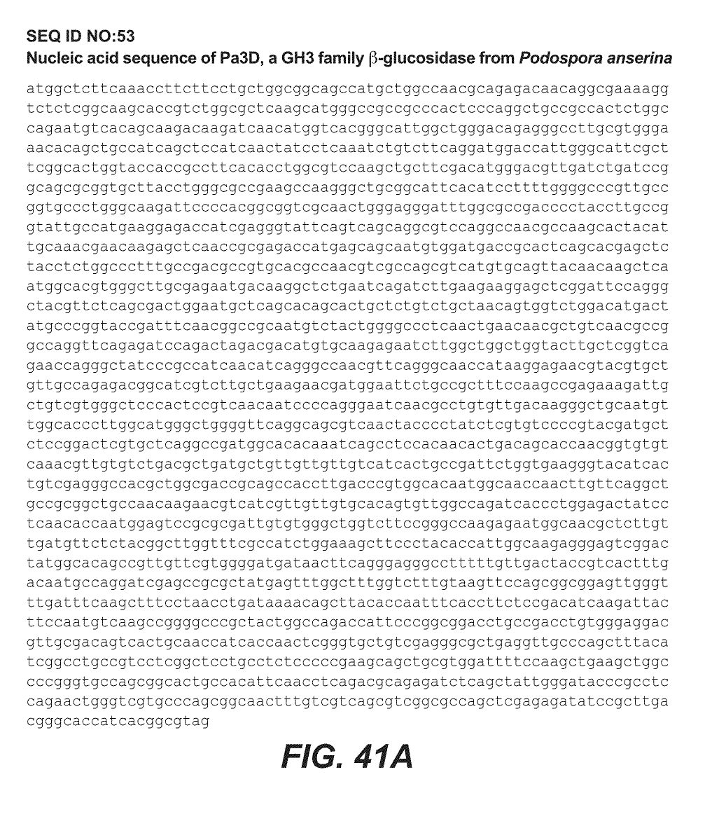

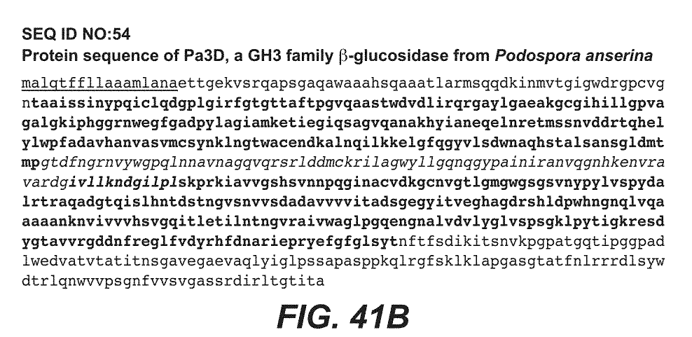

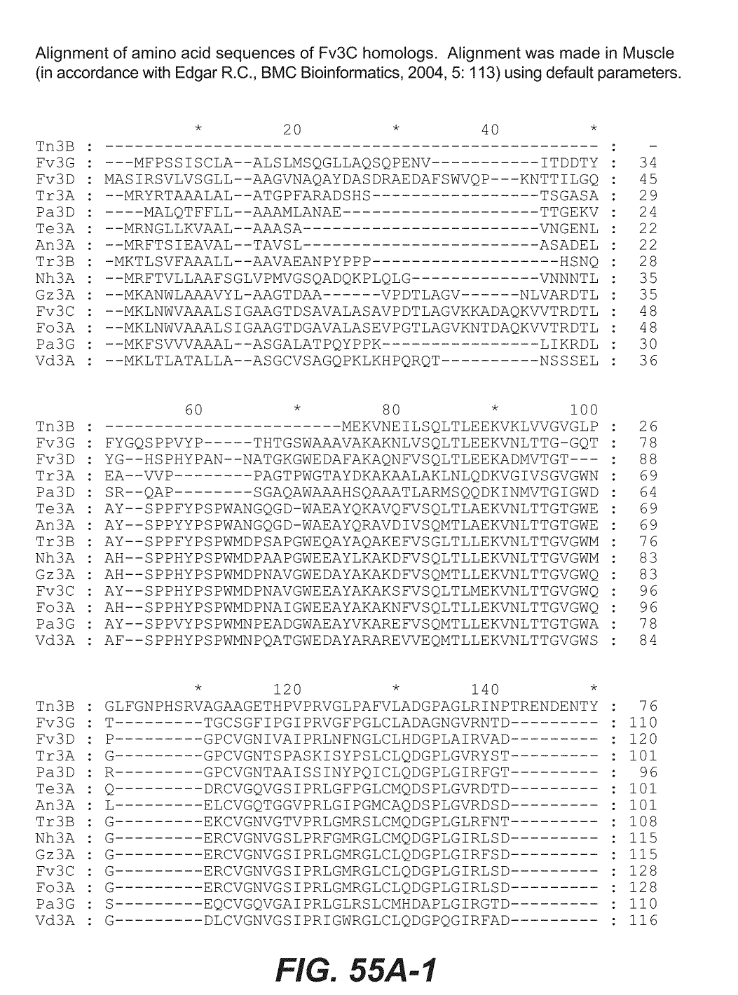

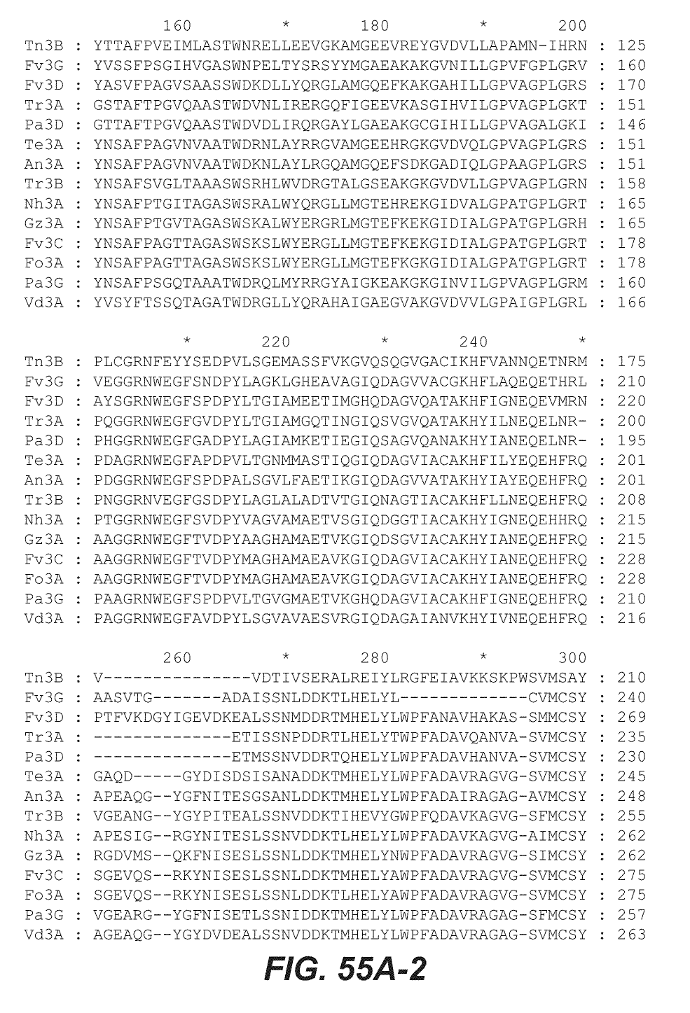

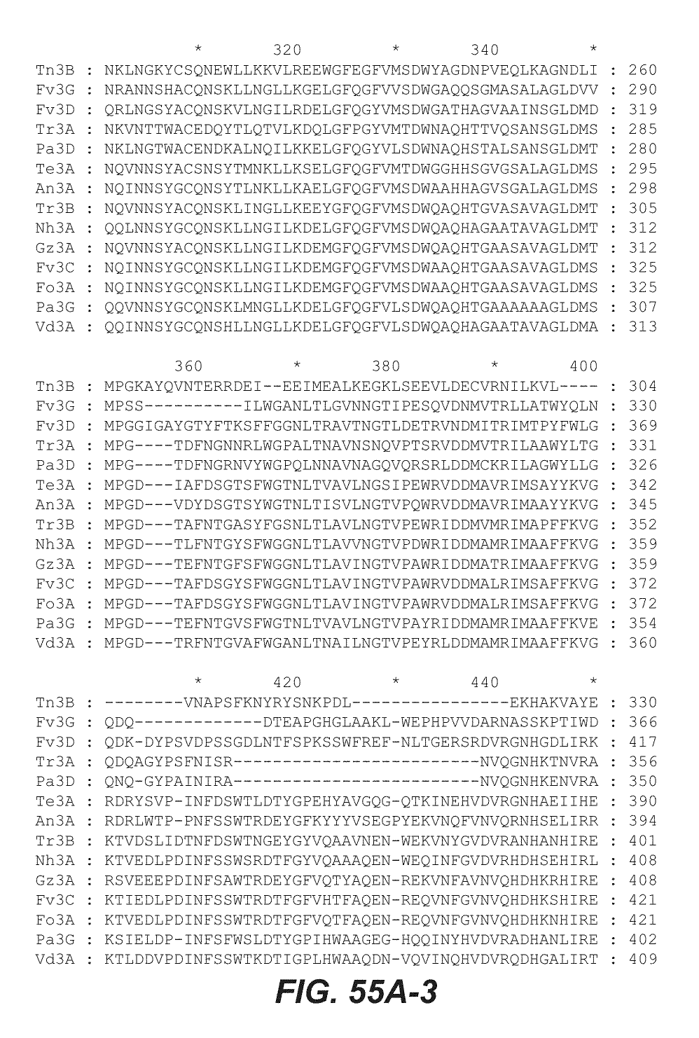

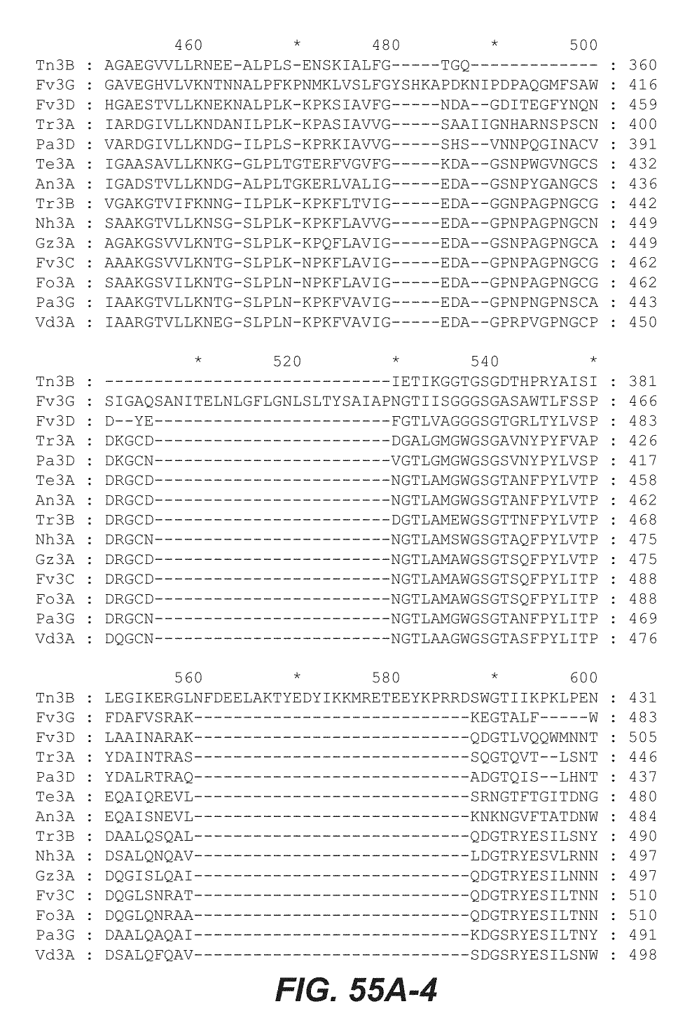

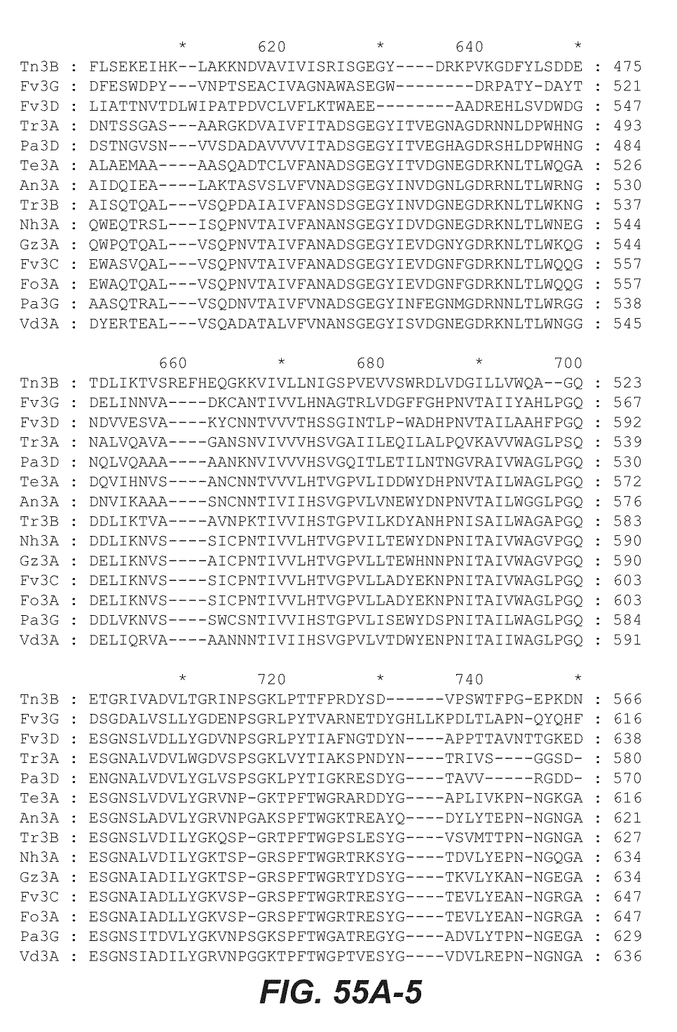

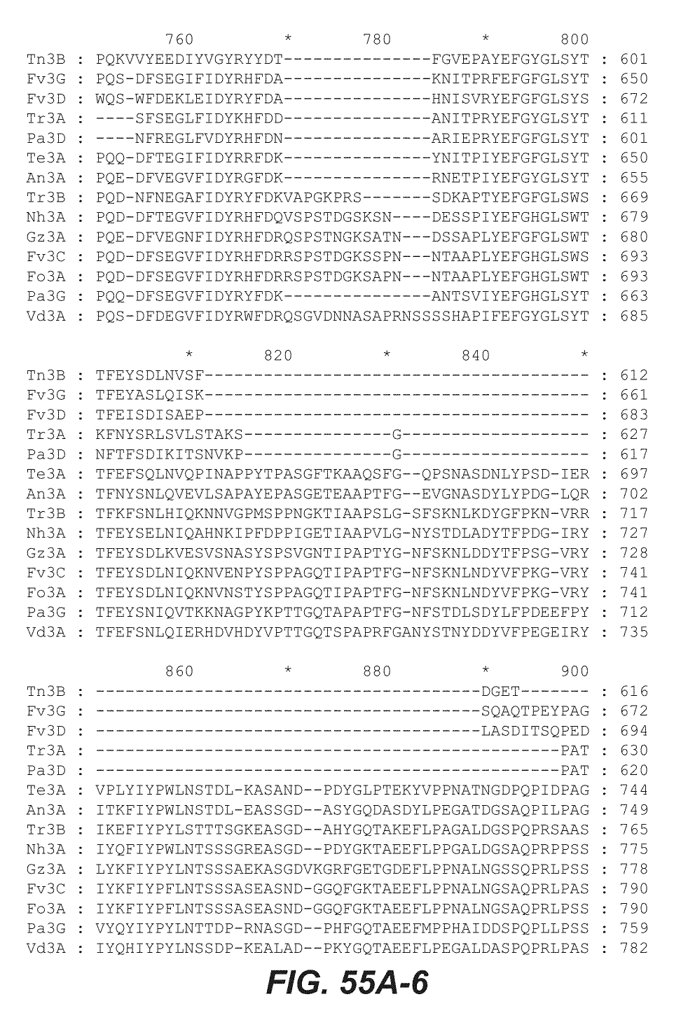

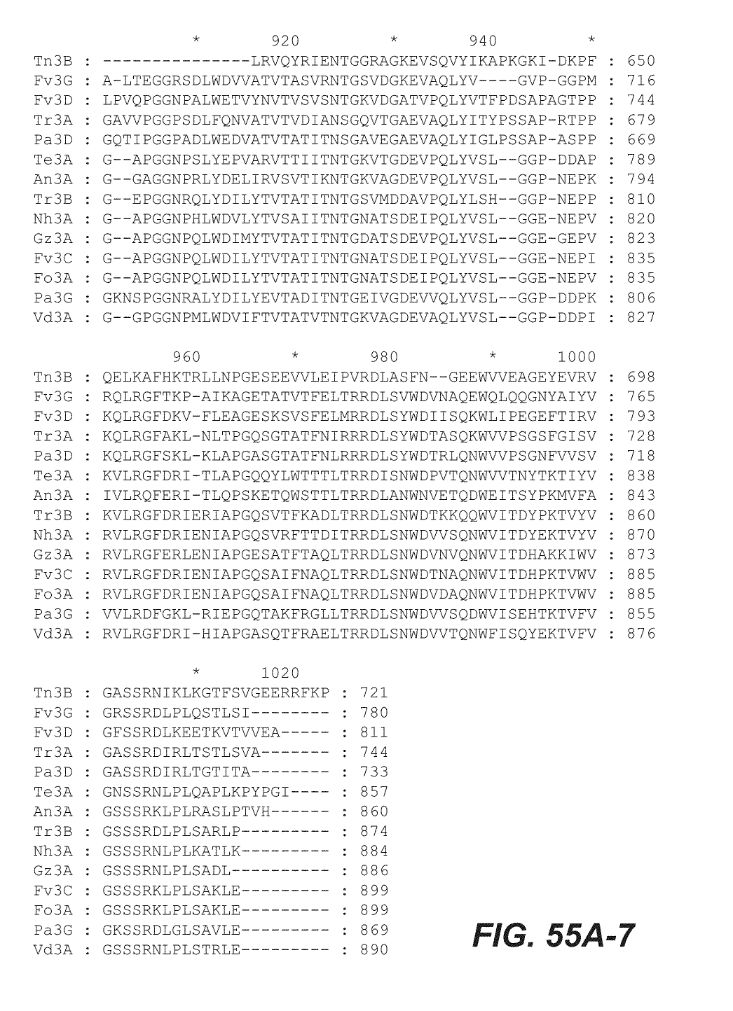

Pa3D: The amino acid sequence of Pa3D (SEQ ID NO:54) is shown in FIGS. 41B and 55. SEQ ID NO:54 is the sequence of the immature Pa3D. Pa3D has a predicted signal sequence corresponding to residues 1 to 17 of SEQ ID NO:2; cleavage of the signal sequence is predicted to yield a mature protein having a sequence corresponding to residues 18 to 733 of SEQ ID NO:54. Signal sequence predictions for this and other polypeptides of the disclosure were made with the SignalP-NN algorithm, herein, (http://www.cbs.dtu.dk). The predicted conserved domain is in boldface type in FIG. 41B. Domain predictions for this and other polypeptides of the disclosure were made based on the Pfam, SMART, or NCBI databases. Pa3D residues E463 and D262 are predicted to function as catalytic acid-base and nucleophile, respectively, based on a sequence alignment of a number of GH3 family β-glucosidases from, e.g., P. anserina (Accession No. XP_001912683), V. dahliae, N. haematococca (Accession No. XP_003045443), G. zeae (Accession No. XP_386781), F. oxysporum (Accession No. BGL FOXG_02349), A. niger (Accession No. CAK48740), T. emersonii (Accession No. AAL69548), T. reesei (Accession No. AAP57755), T. reesei (Accession No. AAA18473), F. verticillioides, and T. neapolitana (Accession No. QOGC07), etc. (see, FIG. 55). As used herein, “a Pa3D polypeptide” refers to a polypeptide and/or a variant thereof comprising a sequence having at least 85%, 86%, 87%, 88%, 89%, 90%, 91%, 92%, 93%, 94%, 95%, 96%, 97%, 98%, 99%, or 100% sequence identity to at least 50, 75, 100, 125, 150, 175, 200, 250, 300, 350, 400, 450, 500, 550, 600, 650 or 700 contiguous amino acid residues among residues 18 to 733 of SEQ ID NO:54. A Pa3D polypeptide preferably is unaltered, as compared to a native Pa3D, at residues E463 and D262. A Pa3D polypeptide is preferably unaltered in at least 70%, 80%, 90%, 95%, 98%, or 99% of the amino acid residues that are conserved among the herein described GH3 family β-glucosidases as shown in the alignment of FIG. 55. A Pa3D polypeptide suitably comprises the entire predicted conserved domains of native Pa3D shown in FIG. 41B. The Pa3D polypeptide of the invention preferably has β-glucosidase activity having at least 90%, 91%, 92%, 93%, 94%, 95%, 96%, 97%, 98%, 99% or 100% sequence identity to the amino acid sequence of SEQ ID NO:54, or to residues (i) 18-282, (ii) 18-601, (iii) 18-733, (iv) 356-601, or (v) 356-733 of SEQ ID NO:54.

In certain embodiments, a Pa3D polypeptide can be a fusion or chimeric polypeptide comprising two or more β-glucosidase sequences, wherein at least one of the β-glucosidase sequences is derived from a Pa3D polypeptide. For example, a Pa3D polypeptide can be a chimeric/fusion polypeptide comprising a polypeptide of at least about 200 amino acid residues in length, derived from a sequence of the same length from the N-terminal of a Pa3D polypeptide or a variant thereof, having at least about 60% sequence identity to SEQ ID NO:54. Alternatively, a Pa3D chimeric/fusion polypeptide can comprise a polypeptide of at least about 50 amino acid residues in length, derived from a sequence of the same length from the C-terminal of a Pa3D polypeptide or a variant thereof, having at least about 60% sequence identity to SEQ ID NO:54. In certain embodiments, a Pa3D chimeric/fusion polypeptide can comprise a loop sequence of about 3, 4, 5, 6, 7, 8, 9, 10, or 11 amino acid residues in length, comprising an amino acid sequence of FDRRSPG (SEQ ID NO:204), or of FD(R/K)YNIT (SEQ ID NO:205).

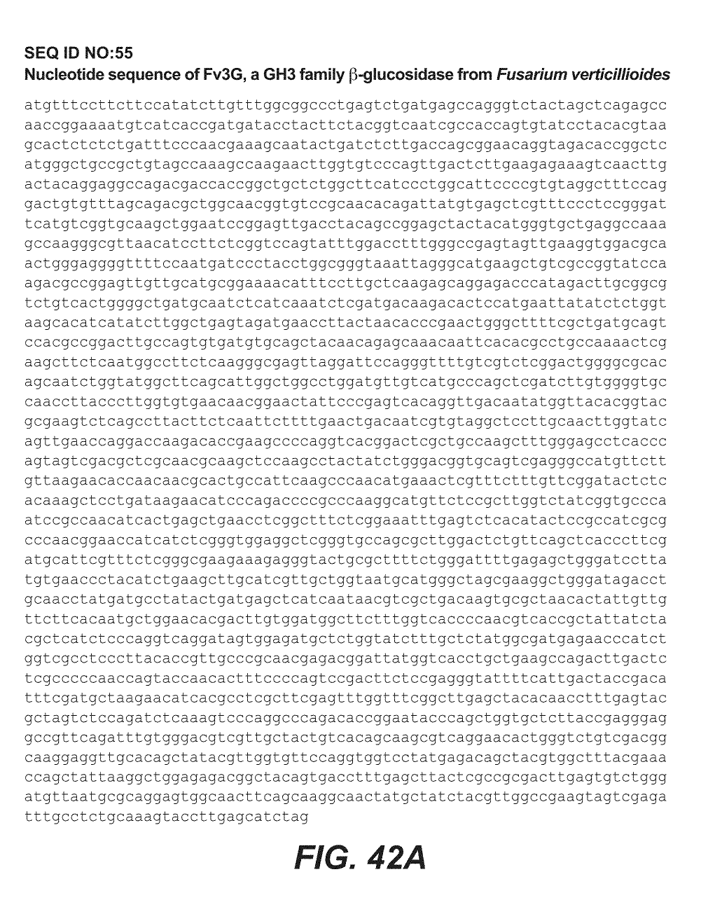

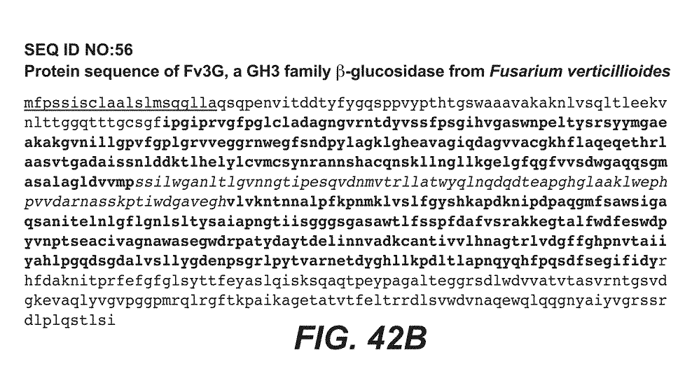

Fv3G: The amino acid sequence of Fv3G (SEQ ID NO:56) is shown in FIGS. 42B and 55. SEQ ID NO:56 is the sequence of the immature Fv3G. Fv3G has a predicted signal sequence corresponding to positions 1 to 21 of SEQ ID NO:56; cleavage of the signal sequence is predicted to yield a mature protein having a sequence corresponding to positions 22 to 780 of SEQ ID NO:56. Signal sequence predictions were, as described above, made with the SignalP-NN algorithm (http://www.cbs.dtu.dk), as they were made for the other polypeptides of the disclosure herein. The predicted conserved domain is in boldface type in FIG. 42B. Domain predictions were made, as they were made with the other polypeptides of the invention herein, based on the Pfam, SMART, or NCBI databases. Fv3G residues E509 and D272 are predicted to function as catalytic acid-base and nucleophile, respectively, based on a sequence alignment of the above-mentioned GH3 glucosidases from, e.g., P. anserina (Accession No. XP_001912683), V. dahliae, N. haematococca (Accession No. XP_003045443), G. zeae (Accession No. XP_386781), F. oxysporum (Accession No. BGL FOXG_02349), A. niger (Accession No. CAK48740), T. emersonii (Accession No. AAL69548), T. reesei (Accession No. AAP57755), T. reesei (Accession No. AAA18473), F. verticillioides, and T. neapolitana (Accession No. QOGC07), etc. (see, FIG. 55). As used herein, “an Fv3Gpolypeptide” refers to a polypeptide and/or a variant thereof comprising a sequence having at least 85%, 86%, 87%, 88%, 89%, 90%, 91%, 92%, 93%, 94%, 95%, 96%, 97%, 98%, 99%, or 100% sequence identity to at least 50, 75, 100, 125, 150, 175, 200, 250, 300, 350, 400, 450, 500, 550, 600, 650, 700, or 750 contiguous amino acid residues among residues 20 to 780 of SEQ ID NO:56. An Fv3G polypeptide preferably is unaltered, as compared to a native Fv3G, at residues E509 and D272. An Fv3G polypeptide is preferably unaltered in at least 70%, 80%, 90%, 95%, 98%, or 99% of the amino acid residues that are conserved among the herein described GH3 family β-glucosidases as shown in the alignment of FIG. 55. An Fv3G polypeptide suitably comprises the entire predicted conserved domains of native Fv3G shown in FIG. 42B. The Fv3G polypeptide of the invention preferably has β-glucosidase activity, having at least 90%, 91%, 92%, 93%, 94%, 95%, 96%, 97%, 98%, 99% or 100% sequence identity to the amino acid sequence of SEQ ID NO:56, or to residues (i) 22-292, (ii) 22-629, (iii) 22-780, (iv) 373-629, or (v) 373-780 of SEQ ID NO:56.

In certain embodiments, an Fv3G polypeptide is a fusion/chimeric polypeptide comprising two or more β-glucosidase sequences, wherein at least one of the β-glucosidase sequences is derived from an Fv3G polypeptide. For example, an Fv3G chimeric/fusion polypeptide can comprise a polypeptide of at least about 200 amino acid residues in length derived from a sequence of the same length from the N-terminal of an Fv3G polypeptide or a variant thereof, having at least about 60% sequence identity to SEQ ID NO:56. For example, an Fv3G chimeric/fusion polypeptide can comprise a polypeptide of at least about 50 amino acid residues in length, derived from a sequence of the same length from the C-terminal of an Fv3G polypeptide or a variant thereof, having at least about 60% sequence identity to SEQ ID NO:56. In certain embodiments, the Fv3G polypeptide further comprises a loop sequence of about 3, 4, 5, 6, 7, 8, 9, 10, or 11 amino acid residues in length, derived from a sequence of the same length of an Fv3G polypeptide or a variant thereof, comprising an amino acid sequence of FDRRSPG (SEQ ID NO:204), or of FD(R/K)YNIT (SEQ ID NO:205).

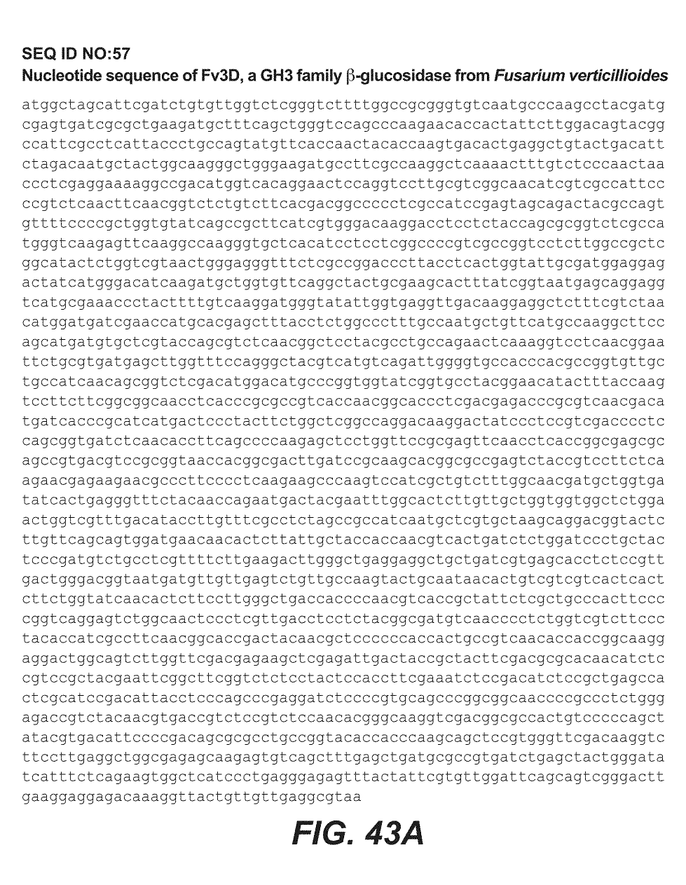

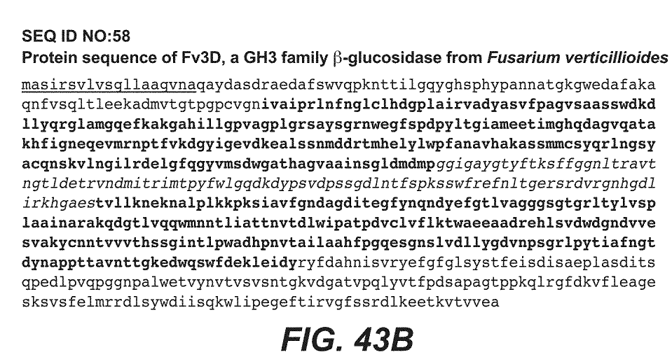

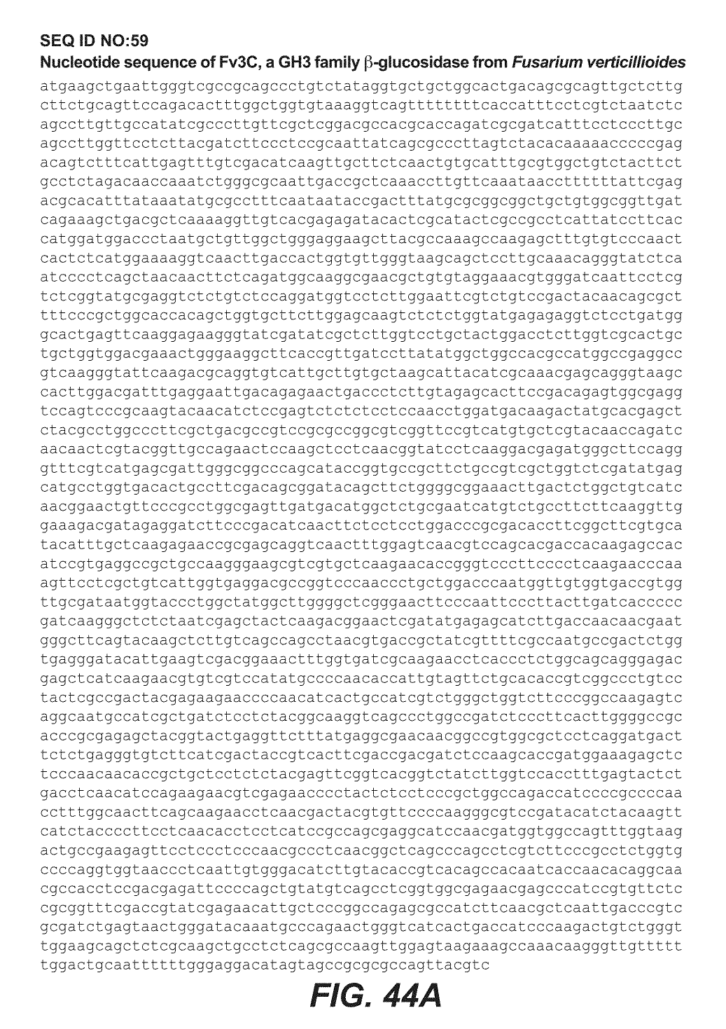

Fv3D: The amino acid sequence of Fv3D (SEQ ID NO:58) is shown in FIGS. 43B and 55. SEQ ID NO:58 is the sequence of the immature Fv3D. Fv3D has a predicted signal sequence corresponding to positions 1 to 19 of SEQ ID NO:58; cleavage of the signal sequence is predicted to yield a mature protein having a sequence corresponding to positions 20 to 811 of SEQ ID NO:58. Signal sequence predictions were made with the SignalP-NN algorithm. The predicted conserved domain is in boldface type in FIG. 43B. Domain predictions were made based on the Pfam, SMART, or NCBI databases. Fv3D residues E534 and D301 are predicted to function as catalytic acid-base and nucleophile, respectively, based on a sequence alignment of the above-mentioned GH3 glucosidases from, e.g., P. anserina (Accession No. XP_001912683), V. dahliae, N. haematococca (Accession No. XP_003045443), G. zeae (Accession No. XP_386781), F. oxysporum (Accession No. BGL FOXG_02349), A. niger (Accession No. CAK48740), T. emersonii (Accession No. AAL69548), T. reesei (Accession No. AAP57755), T. reesei (Accession No. AAA18473), F. verticillioides, and T. neapolitana (Accession No. QOGC07), etc. (see, FIG. 55). As used herein, “an Fv3D polypeptide” refers to a polypeptide and/or a variant thereof comprising a sequence having at least 85%, 86%, 87%, 88%, 89%, 90%, 91%, 92%, 93%, 94%, 95%, 96%, 97%, 98%, 99%, or 100% sequence identity to at least 50, 75, 100, 125, 150, 175, 200, 250, 300, 350, 400, 450, 500, 550, 600, 650, 700, or 750 contiguous amino acid residues among residues 20 to 811 of SEQ ID NO:58. An Fv3D polypeptide preferably is unaltered, as compared to a native Fv3D, at residues E534 and D301. An Fv3D polypeptide is preferably unaltered in at least 70%, 80%, 90%, 95%, 98%, or 99% of the amino acid residues that are conserved among the herein described GH3 family β-glucosidases as shown in the alignment of FIG. 55. An Fv3D polypeptide suitably comprises the entire predicted conserved domains of native Fv3D shown in FIG. 43B. The Fv3D polypeptide of the invention preferably has β-glucosidase activity, having at least 90%, 91%, 92%, 93%, 94%, 95%, 96%, 97%, 98%, 99% or 100% sequence identity to the amino acid sequence of SEQ ID NO:58, or to residues (i) 20-321, (ii) 20-651, (iii) 20-811, (iv) 423-651, or (v) 423-811 of SEQ ID NO:58. The polypeptide suitably has β-glucosidase activity.

In certain embodiments, an Fv3D polypeptide can be a fusion/chimeric polypeptide comprising two or more β-glucosidase sequences, wherein at least one of the β-glucosidase sequences is derived from an Fv3D polypeptide. For example, an Fv3D chimeric/fusion polypeptide can comprise a polypeptide of at least about 200 amino acid residues in length, derived from a sequence of the same length from the N-terminal of an Fv3D polypeptide or a variant thereof, having at least about 60% sequence identity to SEQ ID NO:58. For example, an Fv3D chimeric/fusion polypeptide can comprise a polypeptide of at least about 50 amino acid residues in length, derived from a sequence of the same length from the C-terminal of an Fv3D polypeptide or a variant thereof, having at least about 60% sequence identity to SEQ ID NO:58. In certain embodiments, an Fv3D chimeric/fusion polypeptide can comprise a loop sequence of about 3, 4, 5, 6, 7, 8, 9, 10, or 11 amino acid residues in length, derived from a sequence of the same length of an Fv3D polypeptide or a variant thereof, comprising an amino acid sequence of FDRRSPG (SEQ ID NO:204), or of FD(R/K)YNIT (SEQ ID NO:205).

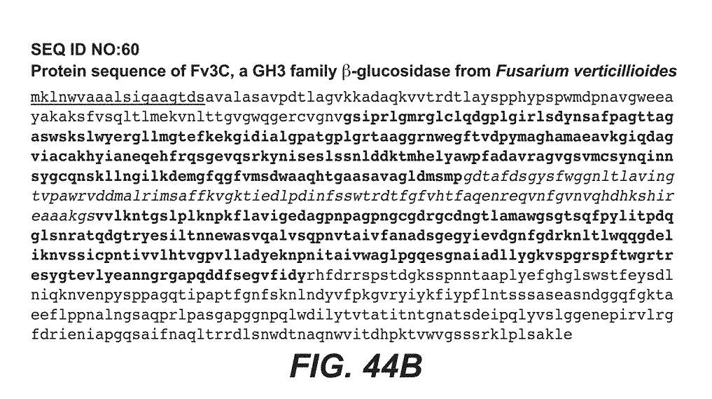

Fv3C: The amino acid sequence of Fv3C (SEQ ID NO:60) is shown in FIGS. 44B and 55. SEQ ID NO:60 is the sequence of the immature Fv3C. Fv3C has a predicted signal sequence corresponding to positions 1 to 19 of SEQ ID NO:60; cleavage of the signal sequence is predicted to yield a mature protein having a sequence corresponding to positions 20 to 899 of SEQ ID NO:60. Signal sequence predictions were made with the SignalP-NN algorithm. The predicted conserved domain is in boldface type in FIG. 44B. Domain predictions were made based on the Pfam, SMART, or NCBI databases. Fv3C residues E536 and D307 are predicted to function as catalytic acid-base and nucleophile, respectively, based on a sequence alignment of the above-mentioned GH3 glucosidases from, e.g., P. anserina (Accession No. XP_001912683), V. dahliae, N. haematococca (Accession No. XP_003045443), G. zeae (Accession No. XP_386781), F. oxysporum (Accession No. BGL FOXG_02349), A. niger (Accession No. CAK48740), T. emersonii (Accession No. AAL69548), T. reesei (Accession No. AAP57755), T. reesei (Accession No. AAA18473), F. verticillioides, and T. neapolitana (Accession No. QOGC07), etc (see, FIG. 55). As used herein, “an Fv3C polypeptide” refers to a polypeptide and/or a variant thereof comprising a sequence having at least 60%, 65%, 70%, 75%, 80%, 85%, 86%, 87%, 88%, 89%, 90%, 91%, 92%, 93%, 94%, 95%, 96%, 97%, 98%, 99%, or 100% sequence identity to at least 50, 75, 100, 125, 150, 175, 200, 250, 300, 350, 400, 450, 500, 550, 600, 650, 700, 750, or 800 contiguous amino acid residues among residues 20 to 899 of SEQ ID NO:60. An Fv3C polypeptide preferably is unaltered, as compared to a native Fv3C, at residues E536 and D307. An Fv3C polypeptide is preferably unaltered in at least 60%, 70%, 80%, 90%, 95%, 98%, or 99% of the amino acid residues that are conserved among the herein described GH3 family β-glucosidases as shown in the alignment of FIG. 55. An Fv3C polypeptide suitably comprises the entire predicted conserved domains of native Fv3C shown in FIG. 44B. The Fv3C polypeptide of the invention preferably has β-glucosidase activity, having at least 90%, 91%, 92%, 93%, 94%, 95%, 96%, 97%, 98%, 99% or 100% sequence identity to the amino acid sequence of SEQ ID NO:60, or to residues (i) 20-327, (ii) 22-600, (iii) 20-899, (iv) 428-899, or (v) 428-660 of SEQ ID NO:60.

In certain embodiments, an Fv3C polypeptide can be a fusion/chimeric polypeptide comprising two or more β-glucosidase sequences, wherein at least one of the β-glucosidase sequences is derived from an Fv3C polypeptide. For example, an Fv3C chimeric/fusion polypeptide can comprise a polypeptide of at least about 200 amino acid residues in length, derived from a sequence of the same length from the N-terminal of an Fv3C polypeptide or a variant thereof, having at least about 60% sequence identity to SEQ ID NO:60. For example, an Fv3C chimeric/fusion polypeptide can comprise a polypeptide of at least about 50 amino acid residues in length, derived from a sequence of the same length from the C-terminal of an Fv3C polypeptide or a variant thereof, having at least about 60% sequence identity to SEQ ID NO:60. In certain embodiments, an Fv3C chimeric/fusion polypeptide can comprise a loop sequence of about 3, 4, 5, 6, 7, 8, 9, 10, or 11 amino acid residues in length, derived from a sequence of the same length of an Fv3C polypeptide or a variant thereof, comprising an amino acid sequence of FDRRSPG (SEQ ID NO:204), or of FD(R/K)YNIT (SEQ ID NO:205)

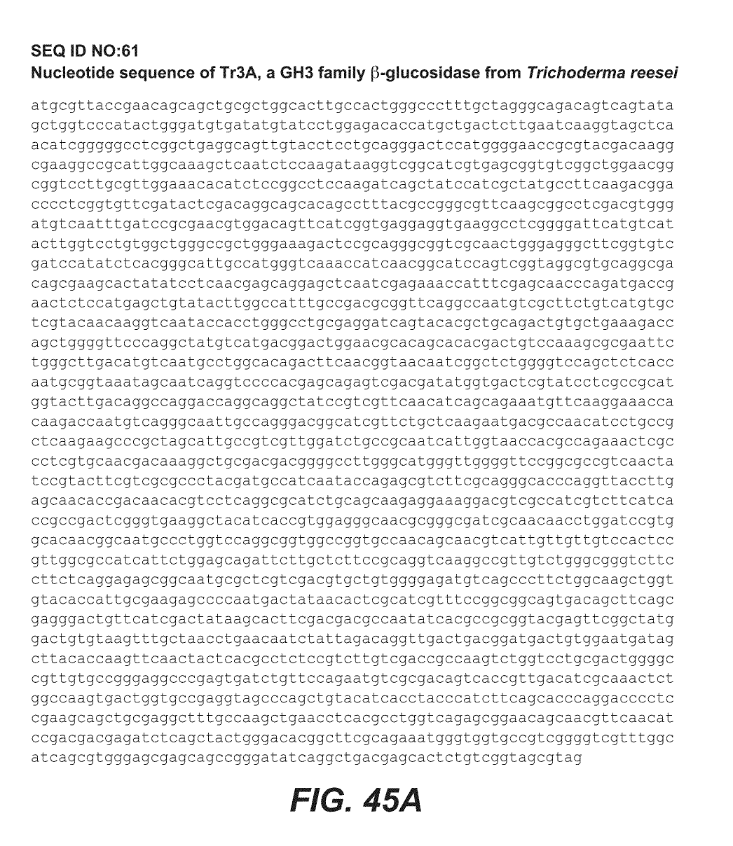

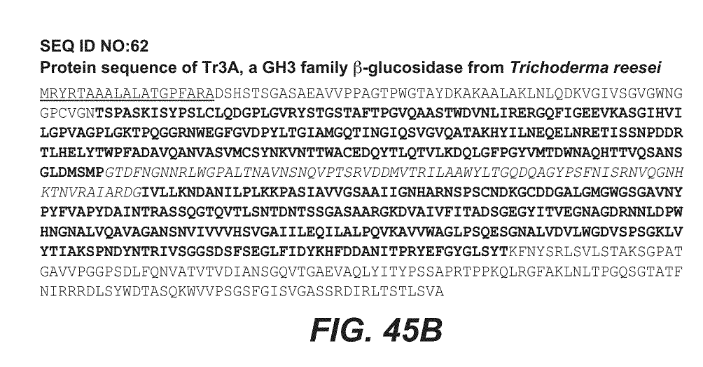

Tr3A: The amino acid sequence of Tr3A (SEQ ID NO:62) is shown in FIGS. 45B and 55. SEQ ID NO:62 is the sequence of the immature Tr3A. Tr3A has a predicted signal sequence corresponding to positions 1 to 19 of SEQ ID NO:62; cleavage of the signal sequence is predicted to yield a mature protein having a sequence corresponding to positions 20 to 744 of SEQ ID NO:62. Signal sequence predictions were made with the SignalP-NN algorithm. The predicted conserved domain is in boldface type in FIG. 45B. Domain predictions were made based on the Pfam, SMART, or NCBI databases. Tr3A residues E472 and D267 are predicted to function as catalytic acid-base and nucleophile, respectively, based on a sequence alignment of the above-mentioned GH3 glucosidases from, e.g., P. anserina (Accession No. XP_001912683), V. dahliae, N. haematococca (Accession No. XP_003045443), G. zeae (Accession No. XP_386781), F. oxysporum (Accession No. BGL FOXG_02349), A. niger (Accession No. CAK48740), T. emersonii (Accession No. AAL69548), T. reesei (Accession No. AAP57755), T. reesei (Accession No. AAA18473), F. verticillioides, and T. neapolitana (Accession No. QOGC07), etc (see, FIG. 55). As used herein, “a Tr3A polypeptide” refers to a polypeptide and/or a variant thereof comprising a sequence having at least 85%, 86%, 87%, 88%, 89%, 90%, 91%, 92%, 93%, 94%, 95%, 96%, 97%, 98%, 99%, or 100% identity to at least 50, 75, 100, 125, 150, 175, 200, 250, 300, 350, 400, 450, 500, 550, 600, 650, or 700 contiguous amino acid residues among residues 20 to 744 of SEQ ID NO:62. A Tr3A polypeptide preferably is unaltered, as compared to a native Tr3A, at residues E472 and D267. A Tr3A polypeptide is preferably unaltered in at least 70%, 80%, 90%, 95%, 98%, or 99% of the amino acid residues that are conserved among the herein described GH3 family β-glucosidases as shown in the alignment of FIG. 55. A Tr3A polypeptide suitably comprises the entire predicted conserved domains of native Tr3A shown in FIG. 45B. The Tr3A polypeptide of the invention preferably has β-glucosidase activity, having at least 90%, 91%, 92%, 93%, 94%, 95%, 96%, 97%, 98%, 99% or 100% identity to the amino acid sequence of SEQ ID NO:62, or to residues (i) 20-287, (ii) 22-611, (iii) 20-744, (iv) 362-611, or (v) 362-744 of SEQ ID NO:62.

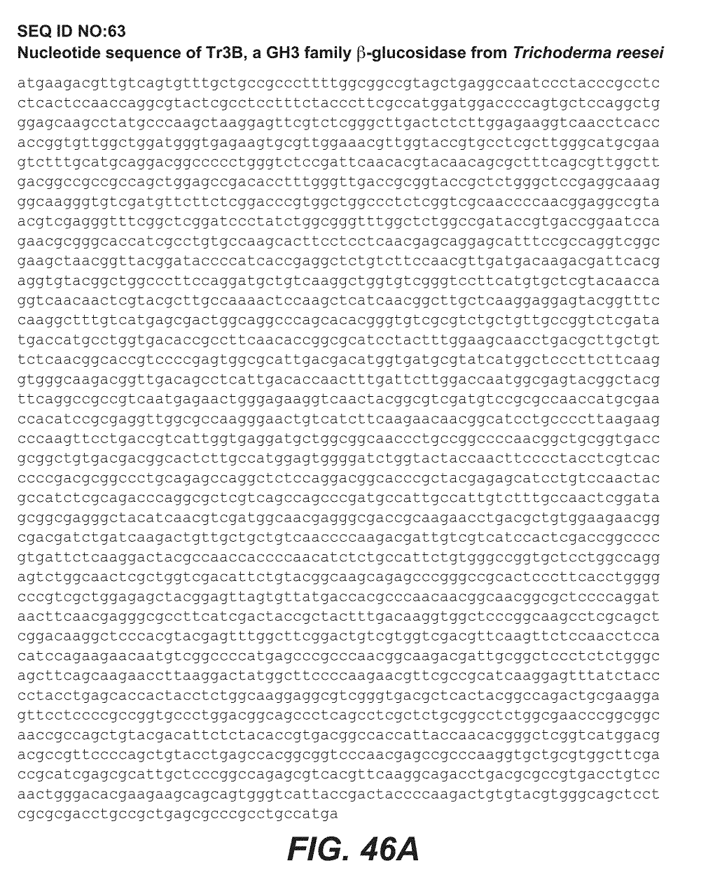

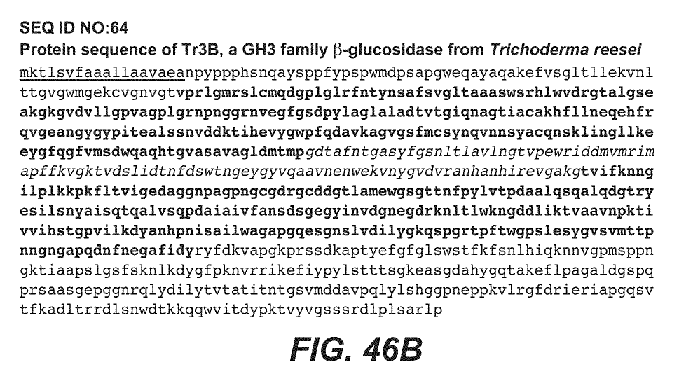

In certain embodiments, a Tr3A polypeptide can be a fusion/chimeric polypeptide comprising two or more β-glucosidase sequences, wherein at least one of the β-glucosidase sequences is derived from a Tr3A polypeptide. For example, a Tr3A chimeric/fusion polypeptide can comprise a polypeptide of at least about 200 amino acid residues in length, derived from a sequence of the same length from the N-terminal of a Tr3A polypeptide or a variant thereof, having at least about 60% sequence identity to SEQ ID NO:62. For example, a Tr3A chimeric/fusion polypeptide can comprise a polypeptide of at least about 50 amino acid residues in length, derived from a sequence of the same length from the C-terminal of a Tr3A polypeptide or a variant thereof, having at least about 60% sequence identity to SEQ ID NO:62. In certain embodiments, a Tr3A chimeric/fusion polypeptide can comprise a loop sequence of about 3, 4, 5, 6, 7, 8, 9, 10, or 11 amino acid residues in length, derived from a sequence of the same length of a Tr3A polypeptide or a variant thereof, comprising an amino acid sequence of FDRRSPG (SEQ ID NO:204), or of FD(R/K)YNIT (SEQ ID NO:205). Tr3B: The amino acid sequence of Tr3B (SEQ ID NO:64) is shown in FIGS. 46B and 55. SEQ ID NO:64 is the sequence of the immature Tr3B. Tr3B has a predicted signal sequence corresponding to positions 1 to 18 of SEQ ID NO:64; cleavage of the signal sequence is predicted to yield a mature protein having a sequence corresponding to positions 19 to 874 of SEQ ID NO:64. Signal sequence predictions were made with the SignalP-NN algorithm. The predicted conserved domain is in boldface type in FIG. 46B. Domain predictions were made based on the Pfam, SMART, or NCBI databases. Tr3B residues E516 and D287 are predicted to function as catalytic acid-base and nucleophile, respectively, based on a sequence alignment of the above-mentioned GH3 glucosidases from, e.g., P. anserina (Accession No. XP_001912683), V. dahliae, N. haematococca (Accession No. XP_003045443), G. zeae (Accession No. XP_386781), F. oxysporum (Accession No. BGL FOXG_02349), A. niger (Accession No. CAK48740), T. emersonii (Accession No. AAL69548), T. reesei (Accession No. AAP57755), T. reesei (Accession No. AAA18473), F. verticillioides, and T. neapolitana (Accession No. QOGC07), etc. (see, FIG. 55). As used herein, “a Tr3B polypeptide” refers to a polypeptide and/or a variant thereof comprising a sequence having at least 85%, 86%, 87%, 88%, 89%, 90%, 91%, 92%, 93%, 94%, 95%, 96%, 97%, 98%, 99%, or 100% identity to at least 50, 75, 100, 125, 150, 175, 200, 250, 300, 350, 400, 450, 500, 550, 600, 650, 700, 750, 800, or 850 contiguous amino acid residues among residues 19 to 874 of SEQ ID NO:64. A Tr3B polypeptide preferably is unaltered, as compared to a native Tr3B, at residues E516 and D287. A Tr3B polypeptide is preferably unaltered in at least 70%, 80%, 90%, 95%, 98%, or 99% of the amino acid residues that are conserved among the herein described GH3 family β-glucosidases as shown in FIG. 55. A Tr3B polypeptide suitably comprises the entire predicted conserved domains of native Tr3B shown in FIG. 46B. The Tr3B polypeptide of the invention preferably has β-glucosidase activity, having at least 90%, 91%, 92%, 93%, 94%, 95%, 96%, 97%, 98%, 99% or 100% identity to the amino acid sequence of SEQ ID NO:64, or to residues (i) 19-307, (ii) 19-640, (iii) 19-874, (iv) 407-640, or (v) 407-874 of SEQ ID NO:64.

In certain embodiments, a Tr3B polypeptide can be a fusion/chimeric polypeptide comprising two or more β-glucosidase sequences, wherein at least one of the β-glucosidase sequences is derived from a Tr3B polypeptide. For example, a Tr3B chimeric/fusion polypeptide can comprise a polypeptide of at least about 200 amino acid residues in length, derived from a sequence of the same length from the N-terminal of a Tr3B polypeptide or a variant thereof, having at least about 60% sequence identity to SEQ ID NO:64. For example, a Tr3B chimeric/fusion polypeptide can comprise a polypeptide of at least about 50 amino acid residues in length, derived from a sequence of the same length from the C-terminal of a Tr3B polypeptide or a variant thereof, having at least about 60% sequence identity to SEQ ID NO:64. In certain embodiments, a Tr3B chimeric/fusion polypeptide can comprise a loop sequence of about 3, 4, 5, 6, 7, 8, 9, 10, or 11 amino acid residues in length, derived from a sequence of the same length of a Tr3B polypeptide or a variant thereof, comprising an amino acid sequence of FDRRSPG (SEQ ID NO:204), or of FD(R/K)YNIT (SEQ ID NO:205).

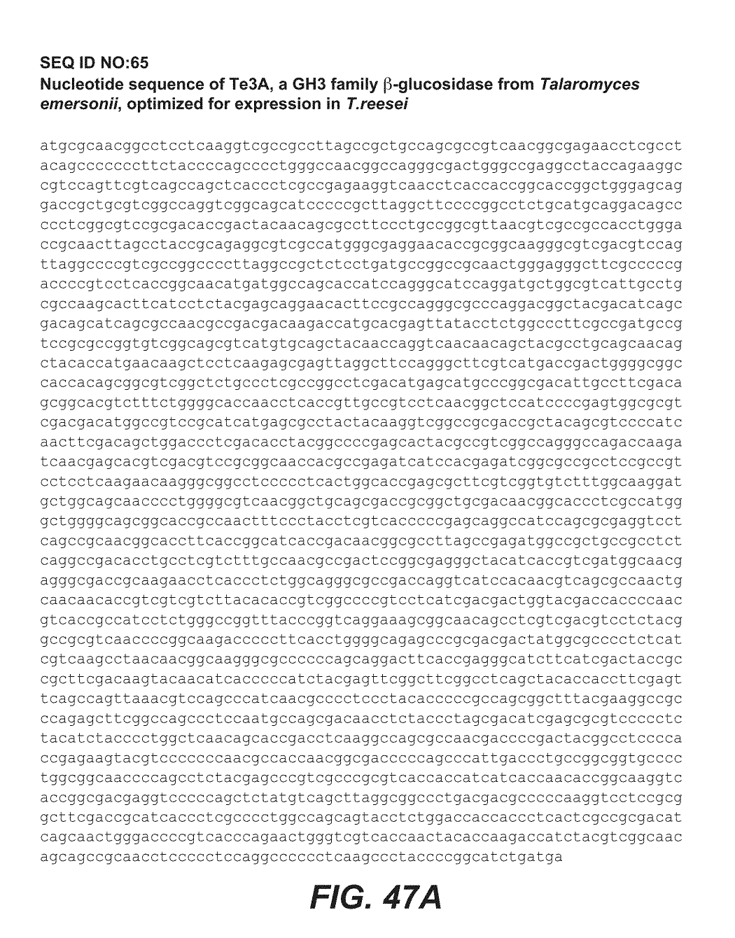

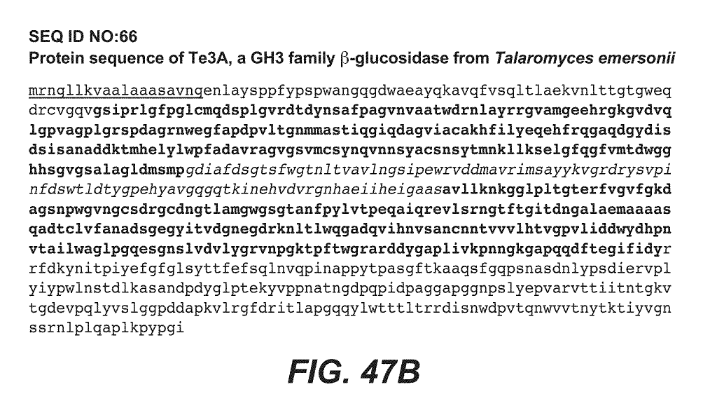

Te3A: The amino acid sequence of Te3A (SEQ ID NO:66) is shown in FIGS. 47B and 55. SEQ ID NO:66 is the sequence of the immature Te3A. Te3A has a predicted signal sequence corresponding to positions 1 to 19 of SEQ ID NO:66; cleavage of the signal sequence is predicted to yield a mature protein having a sequence corresponding to positions 20 to 857 of SEQ ID NO:66. Signal sequence predictions were made with the SignalP-NN algorithm. The predicted conserved domain is in boldface type in FIG. 47B. Domain predictions were made based on the Pfam, SMART, or NCBI databases. Te3A residues E505 and D277 are predicted to function as catalytic acid-base and nucleophile, respectively, based on a sequence alignment of the above-mentioned GH3 glucosidases from, e.g., P. anserina (Accession No. XP_001912683), V. dahliae, N. haematococca (Accession No. XP_003045443), G. zeae (Accession No. XP_386781), F. oxysporum (Accession No. BGL FOXG_02349), A. niger (Accession No. CAK48740), T. emersonii (Accession No. AAL69548), T. reesei (Accession No. AAP57755), T. reesei (Accession No. AAA18473), F. verticillioides, and T. neapolitana (Accession No. QOGC07) etc. (see, FIG. 55). As used herein, “a Te3A polypeptide” refers to a polypeptide and/or a variant thereof comprising a sequence having at least 85%, 86%, 87%, 88%, 89%, 90%, 91%, 92%, 93%, 94%, 95%, 96%, 97%, 98%, 99%, or 100% identity to at least 50, 75, 100, 125, 150, 175, 200, 250, 300, 350, 400, 450, 500, 550, 600, 650, 700, 750, or 800 contiguous amino acid residues among residues 20 to 857 of SEQ ID NO:66. A Te3A polypeptide preferably is unaltered, as compared to a native Te3A, at residues E505 and D277. A Te3A polypeptide is preferably unaltered in at least 70%, 80%, 90%, 95%, 98%, or 99% of the amino acid residues that are conserved among the herein described GH3 family β-glucosidases as shown in FIG. 55. A Te3A polypeptide suitably comprises the entire predicted conserved domains of native Te3A shown in FIG. 47B. The Te3A polypeptide of the invention preferably has β-glucosidase activity having at least 90%, 91%, 92%, 93%, 94%, 95%, 96%, 97%, 98%, 99% or 100% identity to the amino acid sequence of SEQ ID NO:66, or to residues (i) 20-297, (ii) 20-629, (iii) 20-857, (iv) 396-629, or (v) 396-857 of SEQ ID NO:66.

In certain embodiments, a Te3A polypeptide can be a fusion/chimeric polypeptide comprising two or more β-glucosidase sequences, wherein at least one of the β-glucosidase sequences is derived from a Te3A polypeptide. For example, a Te3A chimeric/fusion polypeptide can comprise a polypeptide of at least about 200 amino acid residues in length, derived from a sequence of the same length from the N-terminal of a Te3A polypeptide or a variant thereof, having at least about 60% sequence identity to SEQ ID NO:62. For example, a Te3A chimeric/fusion polypeptide can comprise a polypeptide of at least about 50 amino acid residues in length, derived from a sequence of the same length from the C-terminal of a Te3A polypeptide or a variant thereof, having at least about 60% sequence identity to SEQ ID NO:62. In certain embodiments, a Te3A chimeric/fusion polypeptide can comprise a loop sequence of about 3, 4, 5, 6, 7, 8, 9, 10, or 11 amino acid residues in length, derived from a sequence of the same length of a Te3A polypeptide or a variant thereof, comprising an amino acid sequence of FDRRSPG (SEQ ID NO:204), or of FD(R/K)YNIT (SEQ ID NO:205).

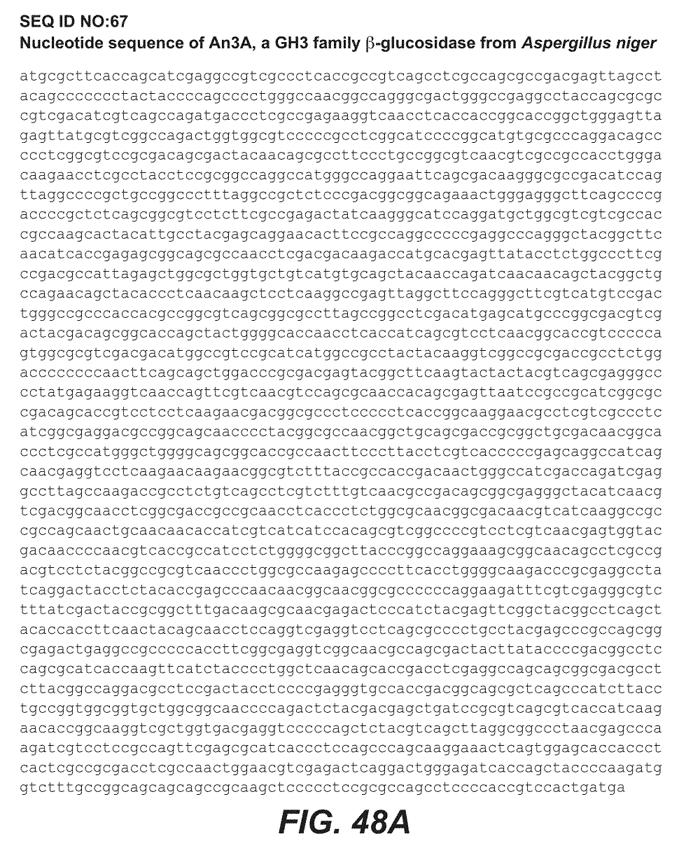

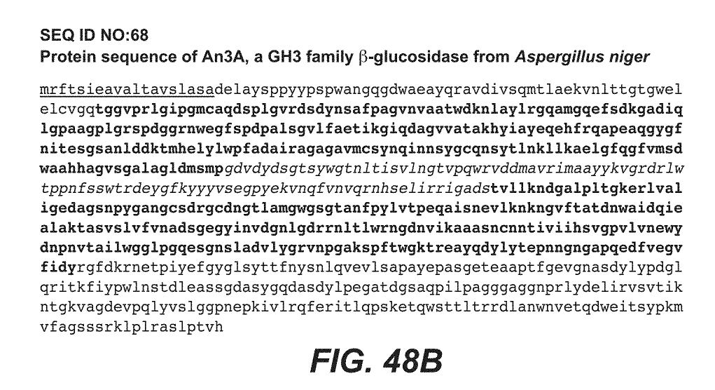

An3A: The amino acid sequence of An3A (SEQ ID NO:68) is shown in FIGS. 48B and 55. SEQ ID NO:6 is the sequence of the immature An3A. An3A has a predicted signal sequence corresponding to positions 1 to 19 of SEQ ID NO:68; cleavage of the signal sequence is predicted to yield a mature protein having a sequence corresponding to positions 20 to 860 of SEQ ID NO:68. Signal sequence predictions were made with the SignalP-NN algorithm. The predicted conserved domain is in boldface type in FIG. 48B. Domain predictions were made based on the Pfam, SMART, or NCBI databases. An3A residues E509 and D277 are predicted to function as catalytic acid-base and nucleophile, respectively, based on a sequence alignment of the above-mentioned GH3 glucosidases from, e.g., P. anserina (Accession No. XP_001912683), V. dahliae, N. haematococca (Accession No. XP_003045443), G. zeae (Accession No. XP_386781), F. oxysporum (Accession No. BGL FOXG_02349), A. niger (Accession No. CAK48740), T. emersonii (Accession No. AAL69548), T. reesei (Accession No. AAP57755), T. reesei (Accession No. AAA18473), F. verticillioides, and T. neapolitana (Accession No. QOGC07), etc. (see, FIG. 55). As used herein, “an An3A polypeptide” refers to a polypeptide and/or a variant thereof comprising a sequence having at least 85%, 86%, 87%, 88%, 89%, 90%, 91%, 92%, 93%, 94%, 95%, 96%, 97%, 98%, 99%, or 100% identity to at least 50, 75, 100, 125, 150, 175, 200, 250, 300, 350, 400, 450, 500, 550, 600, 650, 700, 750, or 800 contiguous amino acid residues among residues 20 to 860 of SEQ ID NO:68. An An3A polypeptide preferably is unaltered, as compared to a native An3A, at residues E509 and D277. An An3A polypeptide is preferably unaltered in at least 70%, 80%, 90%, 95%, 98%, or 99% of the amino acid residues that are conserved among the herein described GH3 family β-glucosidases as shown in FIG. 55. An An3A polypeptide suitably comprises the entire predicted conserved domains of native An3A shown in FIG. 48B. The An3A polypeptide of the invention preferably has β-glucosidase activity, having at least 90%, 91%, 92%, 93%, 94%, 95%, 96%, 97%, 98%, 99% or 100% identity to the amino acid sequence of SEQ ID NO:68, or to residues (i) 20-300, (ii) 20-634, (iii) 20-860, (iv) 400-634, or (v) 400-860 of SEQ ID NO:68.

In certain embodiments, an An3A polypeptide can be a fusion/chimeric polypeptide comprising two or more β-glucosidase sequences, wherein at least one of the β-glucosidase sequences is derived from an An3A polypeptide. For example, an An3A chimeric/fusion polypeptide can comprise a polypeptide of at least about 200 amino acid residues in length, derived from a sequence of the same length from the N-terminal of an An3A polypeptide or a variant thereof, having at least about 60% sequence identity to SEQ ID NO:68. For example, an An3A chimeric/fusion polypeptide can comprise a polypeptide of at least about 50 amino acid residues in length, derived from a sequence of the same length from the C-terminal of an An3A polypeptide or a variant thereof, having at least about 60% sequence identity to SEQ ID NO:68. In certain embodiments, an An3A chimeric/fusion polypeptide can comprise a loop sequence of about 3, 4, 5, 6, 7, 8, 9, 10, or 11 amino acid residues in length, derived from a sequence of the same length of an An3A polypeptide or a variant thereof, comprising an amino acid sequence of FDRRSPG (SEQ ID NO:204), or of FD(R/K)YNIT (SEQ ID NO:205).

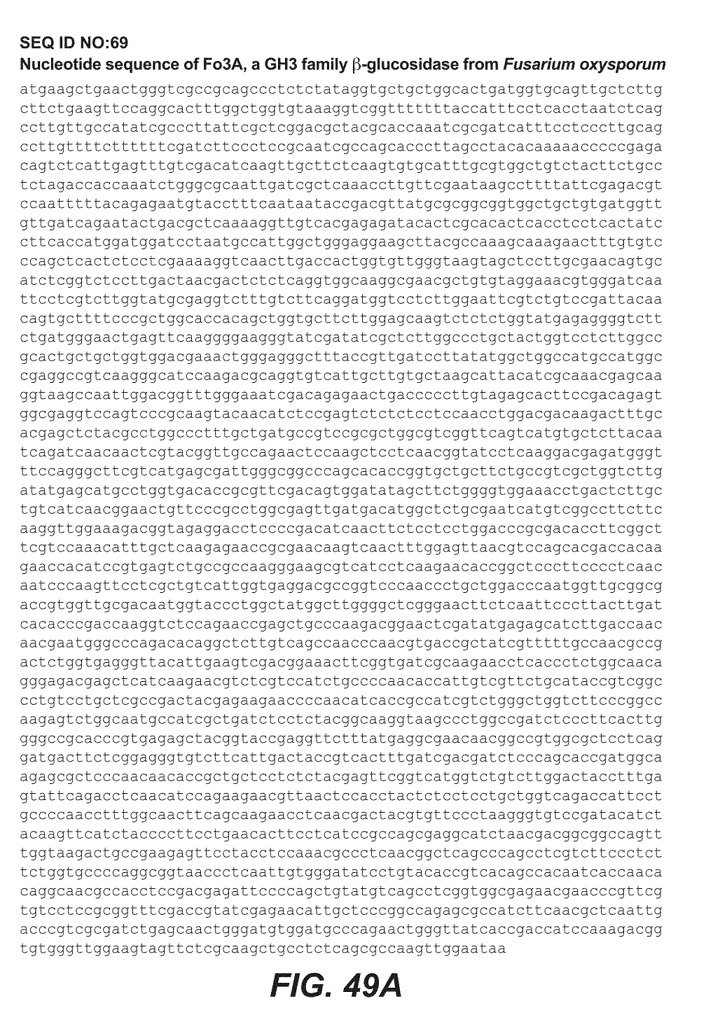

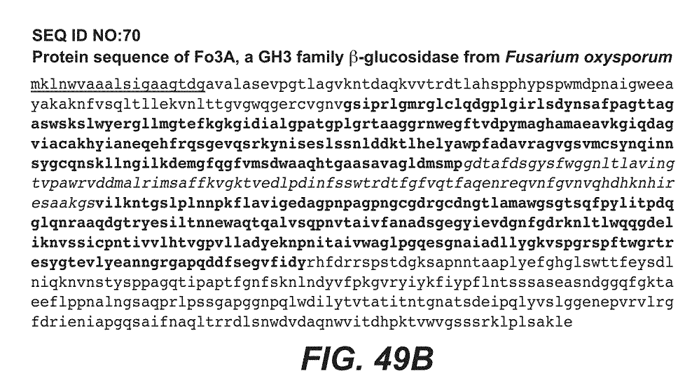

Fo3A: The amino acid sequence of Fo3A (SEQ ID NO:70) is shown in FIGS. 49B and 55. SEQ ID NO:70 is the sequence of the immature Fo3A. Fo3A has a predicted signal sequence corresponding to positions 1 to 19 of SEQ ID NO:70; cleavage of the signal sequence is predicted to yield a mature protein having a sequence corresponding to positions 20 to 899 of SEQ ID NO:70. Signal sequence predictions were made with the SignalP-NN algorithm. The predicted conserved domain is in boldface type in FIG. 49B. Domain predictions were made based on the Pfam, SMART, or NCBI databases. Fo3A residues E536 and D307 are predicted to function as catalytic acid-base and nucleophile, respectively, based on a sequence alignment of the above-mentioned GH3 glucosidases from, e.g., P. anserina (Accession No. XP_001912683), V. dahliae, N. haematococca (Accession No. XP_003045443), G. zeae (Accession No. XP_386781), F. oxysporum (Accession No. BGL FOXG_02349), A. niger (Accession No. CAK48740), T. emersonii (Accession No. AAL69548), T. reesei (Accession No. AAP57755), T. reesei (Accession No. AAA18473), F. verticillioides, and T. neapolitana (Accession No. QOGC07) etc. (see, FIG. 55). As used herein, “an Fo3A polypeptide” refers to a polypeptide and/or a variant thereof comprising a sequence having at least 85%, 86%, 87%, 88%, 89%, 90%, 91%, 92%, 93%, 94%, 95%, 96%, 97%, 98%, 99%, or 100% identity to at least 50, 75, 100, 125, 150, 175, 200, 250, 300, 350, 400, 450, 500, 550, 600, 650, 700, 750, 800, or 850 contiguous amino acid residues among residues 20 to 899 of SEQ ID NO:70. An Fo3A polypeptide preferably is unaltered, as compared to a native Fo3A, at residues E536 and D307. An Fo3A polypeptide is preferably unaltered in at least 70%, 80%, 90%, 95%, 98%, or 99% of the amino acid residues that are conserved among the herein described GH3 β-glucosidases as shown in FIG. 55. An Fo3A polypeptide suitably comprises the entire predicted conserved domains of native Fo3A shown in FIG. 49B. The Fo3A polypeptide of the invention preferably has β-glucosidase activity, having at least 90%, 91%, 92%, 93%, 94%, 95%, 96%, 97%, 98%, 99% or 100% identity to the amino acid sequence of SEQ ID NO:70, or to residues (i) 20-327, (ii) 20-660, (iii) 20-899, (iv) 428-660, or (v) 428-899 of SEQ ID NO:70.

In certain embodiments, an Fo3A polypeptide can be a fusion/chimeric polypeptide comprising two or more β-glucosidase sequences, wherein at least one of the β-glucosidase sequences is derived from an Fo3A polypeptide. For example, an Fo3A chimeric/fusion polypeptide can comprise a polypeptide of at least about 200 amino acid residues in length, derived from a sequence of the same length from the N-terminal of an Fo3A polypeptide or a variant thereof, having at least about 60% sequence identity to SEQ ID NO:70. For example, an Fo3A chimeric/fusion polypeptide can comprise a polypeptide of at least about 50 amino acid residues in length, derived from a sequence of the same length from the C-terminal of an Fo3A polypeptide or a variant thereof, having at least about 60% sequence identity to SEQ ID NO:70. In certain embodiments, an Fo3A chimeric/fusion polypeptide can comprise a loop sequence of about 3, 4, 5, 6, 7, 8, 9, 10, or 11 amino acid residues in length, derived from a sequence of the same length of an Fo3A polypeptide or a variant thereof, comprising an amino acid sequence of FDRRSPG (SEQ ID NO:204), or of FD(R/K)YNIT (SEQ ID NO:205).

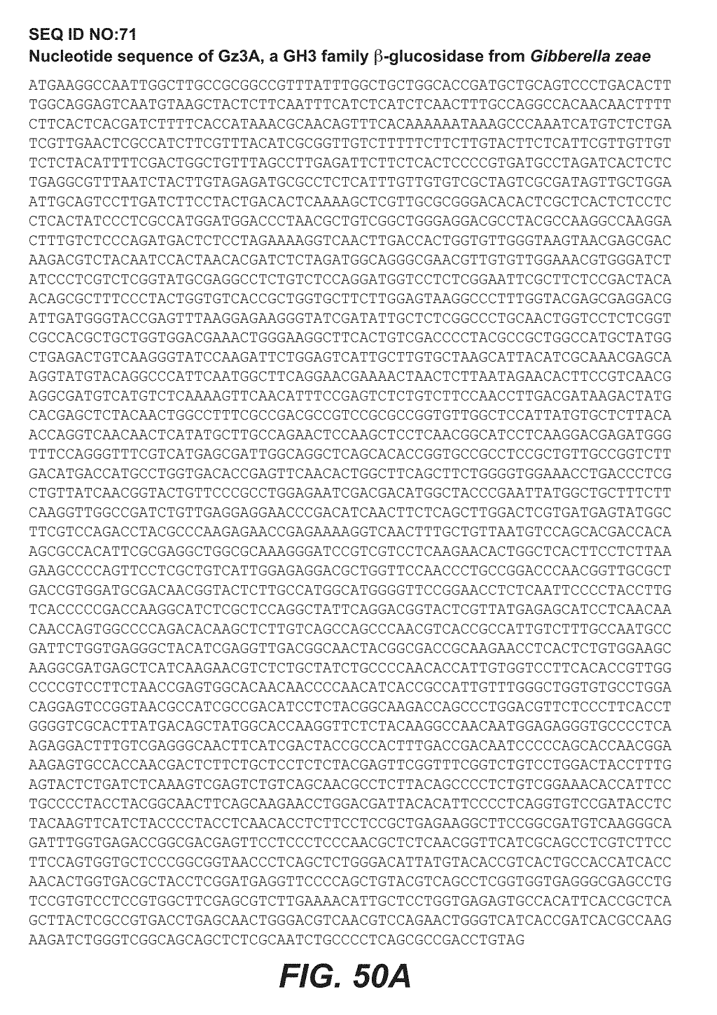

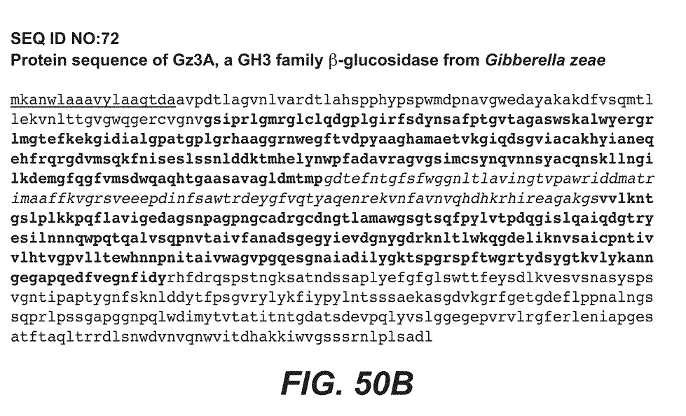

Gz3A: The amino acid sequence of Gz3A (SEQ ID NO:72) is shown in FIGS. 50B and 55. SEQ ID NO:72 is the sequence of the immature Gz3A. Gz3A has a predicted signal sequence corresponding to positions 1 to 18 of SEQ ID NO:72; cleavage of the signal sequence is predicted to yield a mature protein having a sequence corresponding to positions 19 to 886 of SEQ ID NO:72. Signal sequence predictions were made with the SignalP-NN algorithm. The predicted conserved domain is in boldface type in FIG. 50B. Domain predictions were made based on the Pfam, SMART, or NCBI databases. Gz3A residues E523 and D294 are predicted to function as catalytic acid-base and nucleophile, respectively, based on a sequence alignment of the above-mentioned GH3 glucosidases from, e.g., P. anserina (Accession No. XP_001912683), V. dahliae, N. haematococca (Accession No. XP_003045443), G. zeae (Accession No. XP_386781), F. oxysporum (Accession No. BGL FOXG_02349), A. niger (Accession No. CAK48740), T. emersonii (Accession No. AAL69548), T. reesei (Accession No. AAP57755), T. reesei (Accession No. AAA18473), F. verticillioides, and T. neapolitana (Accession No. QOGC07), etc. (see, FIG. 55). As used herein, “a Gz3A polypeptide” refers to a polypeptide and/or a variant thereof comprising a sequence having at least 85%, 86%, 87%, 88%, 89%, 90%, 91%, 92%, 93%, 94%, 95%, 96%, 97%, 98%, 99%, or 100% identity to at least 50, 75, 100, 125, 150, 175, 200, 250, 300, 350, 400, 450, 500, 550, 600, 650, 700, 750, 800, or 850 contiguous amino acid residues among residues 19 to 886 of SEQ ID NO:72. A Gz3A polypeptide preferably is unaltered, as compared to a native Gz3A, at residues E536 and D307. A Gz3A polypeptide is preferably unaltered in at least 70%, 80%, 90%, 95%, 98%, or 99% of the amino acid residues that are conserved among the herein described GH3 family β-glucosidases as shown in FIG. 55. A Gz3A polypeptide suitably comprises the entire predicted conserved domains of native Gz3A shown in FIG. 50B. The Gz3A polypeptide of the invention preferably has β-glucosidase activity, having at least 90%, 91%, 92%, 93%, 94%, 95%, 96%, 97%, 98%, 99% or 100% identity to the amino acid sequence of SEQ ID NO:72, or to residues (i) 19-314, (ii) 19-647, (iii) 19-886, (iv) 415-647, or (v) 415-886 of SEQ ID NO:72.

In certain embodiments, a Gz3A polypeptide can be a fusion/chimeric polypeptide comprising two or more β-glucosidase sequences, wherein at least one of the β-glucosidase sequences is derived from a Gz3A polypeptide. For example, a Gz3A chimeric/fusion polypeptide can comprise a polypeptide of at least about 200 amino acid residues in length, derived from a sequence of the same length from the N-terminal of a Gz3A polypeptide or a variant thereof, having at least about 60% sequence identity to SEQ ID NO:72. For example, a Gz3A chimeric/fusion polypeptide can comprise a polypeptide of at least about 50 amino acid residues in length, derived from a sequence of the same length from the C-terminal of a Gz3A polypeptide or a variant thereof, having at least about 60% sequence identity to SEQ ID NO:72. In certain embodiments, a Gz3A chimeric/fusion polypeptide can comprise a loop sequence of about 3, 4, 5, 6, 7, 8, 9, 10, or 11 amino acid residues in length, derived from a sequence of the same length of a Gz3A polypeptide or a variant thereof, comprising an amino acid sequence of FDRRSPG (SEQ ID NO:204), or of FD(R/K)YNIT (SEQ ID NO:205).

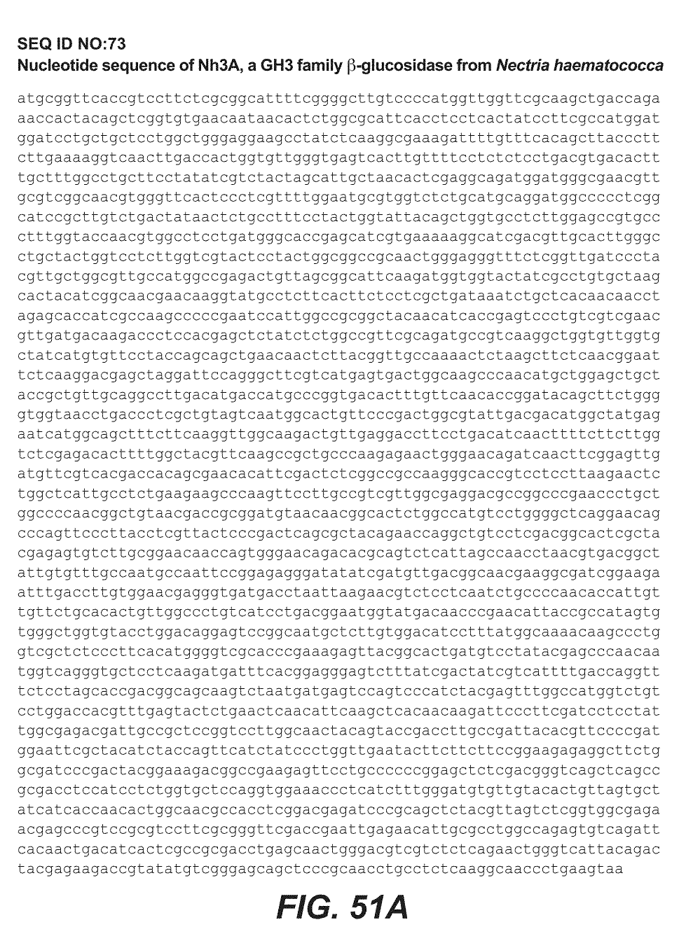

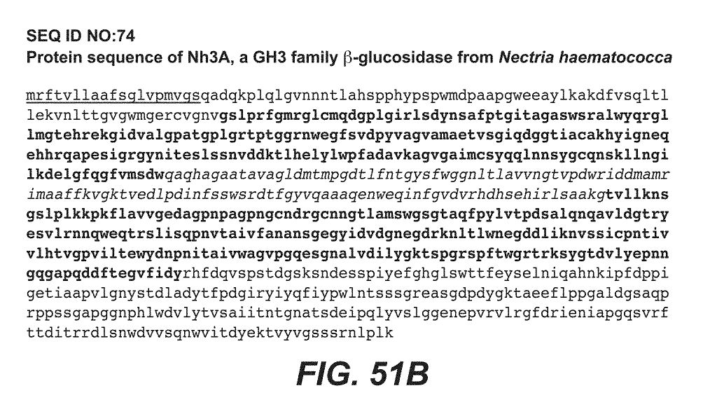

Nh3A: The amino acid sequence of Nh3A (SEQ ID NO:74) is shown in FIGS. 51B and 55. SEQ ID NO:74 is the sequence of the immature Nh3A. Nh3A has a predicted signal sequence corresponding to positions 1 to 19 of SEQ ID NO:74; cleavage of the signal sequence is predicted to yield a mature protein having a sequence corresponding to positions 20 to 880 of SEQ ID NO:74. Signal sequence predictions were made with the SignalP-NN algorithm. The predicted conserved domain is in boldface type in FIG. 51B. Domain predictions were made based on the Pfam, SMART, or NCBI databases. Nh3A residues E523 and D294 are predicted to function as catalytic acid-base and nucleophile, respectively, based on a sequence alignment of the above-mentioned GH3 glucosidases from, e.g., P. anserina (Accession No. XP_001912683), V. dahliae, N. haematococca (Accession No. XP_003045443), G. zeae (Accession No. XP_386781), F. oxysporum (Accession No. BGL FOXG_02349), A. niger (Accession No. CAK48740), T. emersonii (Accession No. AAL69548), T. reesei (Accession No. AAP57755), T. reesei (Accession No. AAA18473), F. verticillioides and T. neapolitana (Accession No. QOGC07), etc. (see, FIG. 55). As used herein, “an Nh3A polypeptide” refers to a polypeptide and/or a variant thereof comprising a sequence having at least 85%, 86%, 87%, 88%, 89%, 90%, 91%, 92%, 93%, 94%, 95%, 96%, 97%, 98%, 99%, or 100% identity to at least 50, 75, 100, 125, 150, 175, 200, 250, 300, 350, 400, 450, 500, 550, 600, 650, 700, 750, 800, or 850 contiguous amino acid residues among residues 20 to 880 of SEQ ID NO:74. An Nh3A polypeptide preferably is unaltered, as compared to a native Nh3A, at residues E523 and D294. An Nh3A polypeptide is preferably unaltered in at least 70%, 80%, 90%, 95%, 98% or 99% of the residues that are conserved among the herein described GH3 family β-glucosidases as shown in FIG. 55. An Nh3A polypeptide suitably comprises the entire predicted conserved domains of native Nh3A shown in FIG. 51B. The Nh3A polypeptide of the invention preferably has β-glucosidase activity, having at least 90%, 91%, 92%, 93%, 94%, 95%, 96%, 97%, 98%, 99% or 100% identity to the amino acid sequence of SEQ ID NO:76, or to residues (i) 20-295, (ii) 20-647, (iii) 20-880, (iv) 414-647, or (v) 414-880 of SEQ ID NO:76.

In certain embodiments, an Nh3A polypeptide can be a fusion/chimeric polypeptide comprising two or more β-glucosidase sequences, wherein at least one of the β-glucosidase sequences is derived from an Nh3A polypeptide. For example, an Nh3A chimeric/fusion polypeptide can comprise a polypeptide of at least about 200 amino acid residues in length, derived from a sequence of the same length from the N-terminal of an Nh3A polypeptide or a variant thereof, having at least about 60% sequence identity to SEQ ID NO:74. For example, an Nh3A chimeric/fusion polypeptide can comprise a polypeptide of at least about 50 amino acid residues in length, derived from a sequence of the same length from the C-terminal of an Nh3A polypeptide or a variant thereof, having at least about 60% sequence identity to SEQ ID NO:74. In certain embodiments, an Nh3A chimeric/fusion polypeptide can comprise a loop sequence of about 3, 4, 5, 6, 7, 8, 9, 10, or 11 amino acid residues in length, derived from a sequence of the same length of an Nh3A polypeptide or a variant thereof, comprising an amino acid sequence of FDRRSPG (SEQ ID NO:204), or of FD(R/K)YNIT (SEQ ID NO:205).

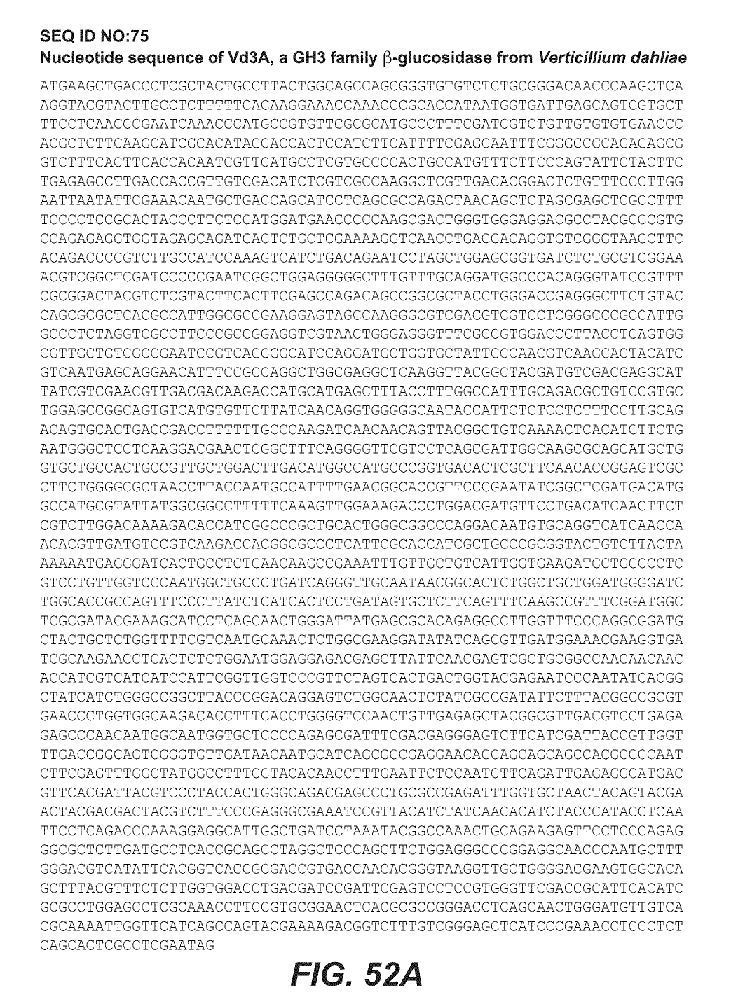

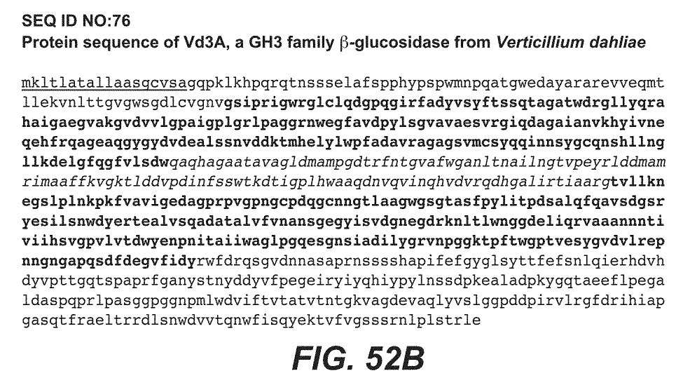

Vd3A: The amino acid sequence of Vd3A (SEQ ID NO:76) is shown in FIGS. 52B and 55. SEQ ID NO:76 is the sequence of the immature Vd3A. Vd3A has a predicted signal sequence corresponding to positions 1 to 18 of SEQ ID NO:76; cleavage of the signal sequence is predicted to yield a mature protein having a sequence corresponding to positions 19 to 890 of SEQ ID NO:76. Signal sequence predictions were made with the SignalP-NN algorithm. The predicted conserved domain is in boldface type in FIG. 52B. Domain predictions were made based on the Pfam, SMART, or NCBI databases. Vd3A was shown to have β-glucosidase activity in, e.g., an enzymatic assay using cNPG and cellobiose, and in hydrolysis of dilute ammonia pretreated corncob as substrates. Vd3A residues E524 and D295 are predicted to function as catalytic acid-base and nucleophile, respectively, based on a sequence alignment of the above-mentioned GH3 glucosidases from, e.g., P. anserina (Accession No. XP_001912683), V. dahliae, N. haematococca (Accession No. XP_003045443), G. zeae (Accession No. XP_386781), F. oxysporum (Accession No. BGL FOXG_02349), A. niger (Accession No. CAK48740), T. emersonii (Accession No. AAL69548), T. reesei (Accession No. AAP57755), T. reesei (Accession No. AAA18473), F. verticillioides, and T. neapolitana (Accession No. QOGC07), etc. (see, FIG. 55). As used herein, “a Vd3A polypeptide” refers to a polypeptide and/or a variant thereof comprising a sequence having at least 85%, 86%, 87%, 88%, 89%, 90%, 91%, 92%, 93%, 94%, 95%, 96%, 97%, 98%, 99%, or 100% identity to at least 50, 75, 100, 125, 150, 175, 200, 250, 300, 350, 400, 450, 500, 550, 600, 650, 700, 750, 800, or 850 contiguous amino acid residues among residues 19 to 890 of SEQ ID NO:76. A Vd3A polypeptide preferably is unaltered, as compared to a native Vd3A, at residues E524 and D295. A Vd3A polypeptide is preferably unaltered in at least 70%, 80%, 90%, 95%, 98%, or 99% of the amino acid residues that are conserved among the herein described GH3 family β-glucosidases as shown in FIG. 55. A Vd3A polypeptide suitably comprises the entire predicted conserved domains of native Vd3A shown in FIG. 52B.The Vd3A polypeptide of the invention preferably has β-glucosidase activity having at least 90%, 91%, 92%, 93%, 94%, 95%, 96%, 97%, 98%, 99% or 100% identity to the amino acid sequence of SEQ ID NO:76, or to residues (i) 19-296, (ii) 19-649, (iii) 19-890, (iv) 415-649, or (v) 415-890 of SEQ ID NO:76.

In certain embodiments, a Vd3A polypeptide can be a fusion/chimeric polypeptide comprising two or more β-glucosidase sequences, wherein at least one of the β-glucosidase sequences is derived from a Vd3A polypeptide. For example, a Vd3A chimeric/fusion polypeptide can comprise a polypeptide of at least about 200 amino acid residues in length, derived from a sequence of the same length from the N-terminal of a Vd3A polypeptide or a variant thereof, having at least about 60% sequence identity to SEQ ID NO:76. For example, a Vd3A chimeric/fusion polypeptide can comprise a polypeptide of at least about 50 amino acid residues in length, derived from a sequence of the same length from the C-terminal of a Vd3A polypeptide or a variant thereof, having at least about 60% sequence identity to SEQ ID NO:76. In certain embodiments, a Vd3A chimeric/fusion polypeptide can comprise a loop sequence of about 3, 4, 5, 6, 7, 8, 9, 10, or 11 amino acid residues in length, derived from a sequence of the same length of a Vd3A polypeptide or a variant thereof, comprising an amino acid sequence of FDRRSPG (SEQ ID NO:204), or of FD(R/K)YNIT (SEQ ID NO:205)

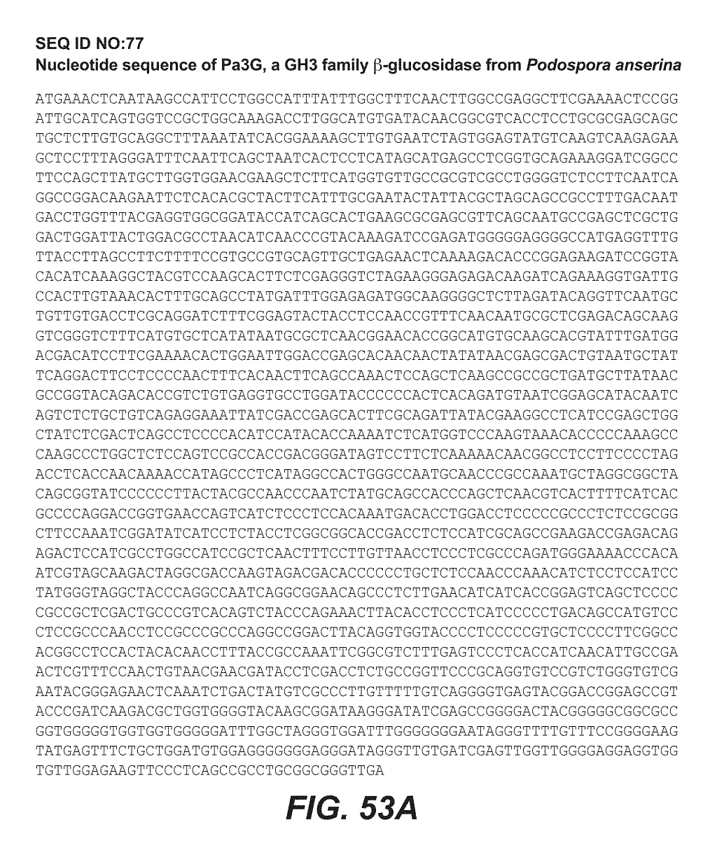

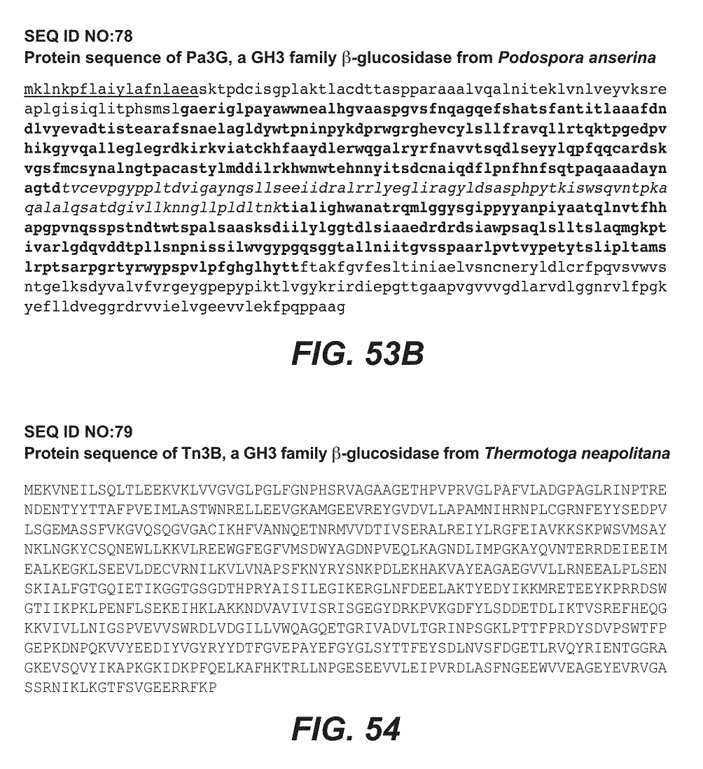

Pa3G: The amino acid sequence of Pa3G (SEQ ID NO:78) is shown in FIGS. 53B and 55. SEQ ID NO:78 is the sequence of the immature Pa3G. Pa3G has a predicted signal sequence corresponding to positions 1 to 19 of SEQ ID NO:78; cleavage of the signal sequence is predicted to yield a mature protein having a sequence corresponding to positions 20 to 805 of SEQ ID NO:78. Signal sequence predictions were made with the SignalP-NN algorithm. The predicted conserved domain is in boldface type in FIG. 53B. Domain predictions were made based on the Pfam, SMART, or NCBI databases. Pa3G residues E517 and D289 are predicted to function as catalytic acid-base and nucleophile, respectively, based on a sequence alignment of the above-mentioned GH3 glucosidases from, e.g., P. anserina (Accession No. XP_001912683), V. dahliae, N. haematococca (Accession No. XP_003045443), G. zeae (Accession No. XP_386781), F. oxysporum (Accession No. BGL FOXG_02349), A. niger (Accession No. CAK48740), T. emersonii (Accession No. AAL69548), T. reesei (Accession No. AAP57755), T. reesei (Accession No. AAA18473), F. verticillioides, and T. neapolitana (Accession No. QOGC07), etc. (see, FIG. 55). As used herein, “a Pa3G polypeptide” refers to a polypeptide and/or a variant thereof comprising a sequence having at least 85%, 86%, 87%, 88%, 89%, 90%, 91%, 92%, 93%, 94%, 95%, 96%, 97%, 98%, 99%, or 100% identity to at least 50, 75, 100, 125, 150, 175, 200, 250, 300, 350, 400, 450, 500, 550, 600, 650, 700, or 750 contiguous amino acid residues among residues 20 to 805 of SEQ ID NO:78. A Pa3G polypeptide preferably is unaltered, as compared to a native Pa3G, at residues E517 and D289. A Pa3G polypeptide is preferably unaltered in at least 70%, 80%, 90%, 95%, 98%, or 99% of the amino acid residues that are conserved among the herein described GH3 family β-glucosidases as shown in FIG. 55. A Pa3G polypeptide suitably comprises the entire predicted conserved domains of native Pa3G shown in FIG. 53B. The Pa3G polypeptide of the invention preferably has β-glucosidase activity having at least 90%, 91%, 92%, 93%, 94%, 95%, 96%, 97%, 98%, 99% or 100% identity to the amino acid sequence of SEQ ID NO:78, or to residues (i) 20-354, (ii) 20-660, (iii) 20-805, (iv) 449-660, or (v) 449-805 of SEQ ID NO:78.

In certain embodiments, a Pa3G polypeptide can be a fusion/chimeric polypeptide comprising two or more β-glucosidase sequences, wherein at least one of the β-glucosidase sequences is derived from a Pa3G polypeptide. For example, a Pa3G chimeric/fusion polypeptide can comprise a polypeptide of at least about 200 amino acid residues in length, derived from a sequence of the same length from the N-terminal of a Pa3G polypeptide or a variant thereof, having at least about 60% sequence identity to SEQ ID NO:78. For example, a Pa3G chimeric/fusion polypeptide can comprise a polypeptide of at least about 50 amino acid residues in length, derived from a sequence of the same levgth from the C-terminal of a Pa3G polypeptide or a variant thereof, having at least about 60% sequence identity to SEQ ID NO:78. In certain embodiments, a Pa3G chimeric/fusion polypeptide can comprise a loop sequence of about 3, 4, 5, 6, 7, 8, 9, 10, or 11 amino acid residues in length, derived from a sequence of the same length of a Pa3G polypeptide or a variant thereof, comprising an amino acid sequence of FDRRSPG (SEQ ID NO:204), or of FD(R/K)YNIT (SEQ ID NO:205).

Tn3B: The amino acid sequence of Tn3B (SEQ ID NO:79) is shown in FIGS. 54 and 55. SEQ ID NO:79 is the sequence of the immature Tn3B. The SignalP-NN algorithm (http://www.cbs.dtu.dk) did not provide a predicted signal sequence. Tn3B residues E458 and D242 are predicted to function as catalytic acid-base and nucleophile, respectively, based on a sequence alignment of the above-mentioned GH3 glucosidases, e.g., P. anserina (Accession No. XP_001912683), V. dahliae, N. haematococca (Accession No. XP_003045443), G. zeae (Accession No. XP_386781), F. oxysporum (Accession No. BGL FOXG_02349), A. niger (Accession No. CAK48740), T. emersonii (Accession No. AAL69548), T. reesei (Accession No. AAP57755), T. reesei (Accession No. AAA18473), F. verticillioides, and T. neapolitana (Accession No. QOGC07), etc. (see, FIG. 55). As used herein, “a Tn3B polypeptide” refers to a polypeptide and/or a variant thereof comprising a sequence having at least 85%, 86%, 87%, 88%, 89%, 90%, 91%, 92%, 93%, 94%, 95%, 96%, 97%, 98%, 99%, or 100% identity to at least 50, 75, 100, 125, 150, 175, 200, 250, 300, 350, 400, 450, 500, 550, 600, 650, 700, or 750 contiguous amino acid residues of SEQ ID NO:79. A Tn3B polypeptide preferably is unaltered, as compared to a native Tn3B, at residues E458 and D242. A Tn3B polypeptide is preferably unaltered in at least 70%, 80%, 90%, 95%, 98%, or 99% of the amino acid residues that are conserved among the herein described GH3 family β-glucosidases as shown in the alignment of FIG. 55. A Tn3B polypeptide suitably comprises the entire predicted conserved domains of native Tn3B shown in FIG. 54. The Tn3B polypeptide of the invention preferably has β-glucosidase activity.

In certain embodiments, a Tn3B polypeptide can be a fusion/chimeric polypeptide comprising two or more β-glucosidase sequences, wherein at least one of the β-glucosidase sequences is derived from a Tn3B polypeptide. For example, a Tn3B chimeric/fusion polypeptide can comprise a polypeptide of at least about 200 amino acid residues in length, derived from a sequence of the same length from the N-terminal of a a Tn3B polypeptide or a variant thereof, having at least about 60% sequence identity to SEQ ID NO:79. For example, a Tn3B chimeric/fusion polypeptide can comprise a polypeptide of at least about 50 amino acid residues in length, derived from a sequence of the same length from the C-terminal of a Tn3B polypeptide or a variant thereof, having at least about 60% sequence identity to SEQ ID NO:79. In certain embodiments, a Tn3B chimeric/fusion polypeptide can comprise a loop sequence of about 3, 4, 5, 6, 7, 8, 9, 10, or 11 amino acid residues in length, derived from a sequence of the same length of a Tn3B polypeptide or a variant thereof, comprising an amino acid sequence of FDRRSPG (SEQ ID NO:204), or of FD(R/K)YNIT (SEQ ID NO:205).

Accordingly, the present disclosure provides a number of isolated, synthetic, or recombinant polypeptides or variants as described below:

(1) a polypeptide having at least 80%, 85%, 90%, 91%, 92%, 93%, 94%, 95%, 96%, 97%, 98%, 99%, or 100% identity to the amino acid sequence corresponding to positions (i) 24 to 766 of SEQ ID NO:2; (ii) 73 to 321 of SEQ ID NO:2; (iii) 73 to 394 of SEQ ID NO:2; (iv) 395 to 622 of SEQ ID NO:2; (v) 24 to 622 of SEQ ID NO:2; or (iv) 73 to 622 of SEQ ID NO:2; the polypeptide has β-xylosidase activity; or

(2) a polypeptide having at least 80%, 85%, 90%, 91%, 92%, 93%, 94%, 95%, 96%, 97%, 98%, 99%, or 100% identity to the amino acid sequence corresponding to positions (i) 21 to 445 of SEQ ID NO:4; (ii) 21 to 301 of SEQ ID NO:4; (iii) 21 to 323 of SEQ ID NO:4; (iv) 21 to 444 of SEQ ID NO:4; (v) 302 to 444 of SEQ ID NO:4; (vi) 302 to 445 of SEQ ID NO:4; (vii) 324 to 444 of SEQ ID NO:4; or (viii) 324 to 445 of SEQ ID NO:4; the polypeptide has β-xylosidase activity; or

(3) a polypeptide having at least 80%, 85%, 90%, 91%, 92%, 93%, 94%, 95%, 96%, 97%, 98%, 99%, or 100% identity to the amino acid sequence corresponding to positions (i) 19 to 530 of SEQ ID NO:6; (ii) 29 to 530 of SEQ ID NO:6; (iii) 19 to 300 of SEQ ID NO:6; or (iv) 29 to 300 of SEQ ID NO:6; the polypeptide has β-xylosidase activity; or

(4) a polypeptide having at least 80%, 85%, 90%, 91%, 92%, 93%, 94%, 95%, 96%, 97%, 98%, 99%, or 100% identity to the amino acid sequence corresponding to positions (i) 20 to 439 of SEQ ID NO:8; (ii) 20 to 291 of SEQ ID NO:8; (iii) 145 to 291 of SEQ ID NO:8; or (iv) 145 to 439 of SEQ ID NO:8; the polypeptide has β-xylosidase activity; or

(5) a polypeptide havingat least 80%, 85%, 90%, 91%, 92%, 93%, 94%, 95%, 96%, 97%, 98%, 99%, or 100% identity to the amino acid sequence corresponding to positions (i) 23 to 449 of SEQ ID NO:10; (ii) 23 to 302 of SEQ ID NO:10; (iii) 23 to 320 of SEQ ID NO:10; (iv) 23 to 448 of SEQ ID NO:10; (v) 303 to 448 of SEQ ID NO:10; (vi) 303 to 449 of SEQ ID NO:10; (vii) 321 to 448 of SEQ ID NO:10; or (viii) 321 to 449 of SEQ ID NO:10; the polypeptide has β-xylosidase activity; or

(6) a polypeptide having at least 80%, 85%, 90%, 91%, 92%, 93%, 94%, 95%, 96%, 97%, 98%, 99%, or 100% identity to the amino acid sequence corresponding to positions (i) 17 to 574 of SEQ ID NO:12; (ii) 27 to 574 of SEQ ID NO:12; (iii) 17 to 303 of SEQ ID NO:12; or (iv) 27 to 303 of SEQ ID NO:12; the polypeptide has β-xylosidase activity and L-α-arabinofuranosidase activity; or

(7) a polypeptide having at least 80%, 85%, 90%, 91%, 92%, 93%, 94%, 95%, 96%, 97%, 98%, 99%, or 100% identity to the amino acid sequence corresponding to positions (i) 21 to 676 of SEQ ID NO:14; (ii) 21 to 652 of SEQ ID NO:14; (iii) 469 to 652 of SEQ ID NO:14; or (iv) 469 to 676 of SEQ ID NO:14; the polypeptide has both β-xylosidase activity and L-α-arabinofuranosidase activity; or

(8) a polypeptide having at least 80%, 85%, 90%, 91%, 92%, 93%, 94%, 95%, 96%, 97%, 98%, 99%, or 100% identity to the amino acid sequence corresponding to positions (i) 19 to 340 of SEQ ID NO:16; (ii) 53 to 340 of SEQ ID NO:16; (iii) 19 to 383 of SEQ ID NO:16; or (iv) 53 to 383 of SEQ ID NO:16; the polypeptide has β-xylosidase activity; or

(9) a polypeptide having at least 80%, 85%, 90%, 91%, 92%, 93%, 94%, 95%, 96%, 97%, 98%, 99%, or 100% identity to the amino acid sequence corresponding to positions (i) 21 to 341 of SEQ ID NO:18; (ii) 107 to 341 of SEQ ID NO:18; (iii) 21 to 348 of SEQ ID NO:18; or (iv) 107 to 348 of SEQ ID NO:18; the polypeptide has β-xylosidase activity; or

(10) a polypeptide having at least 80%, 85%, 90%, 91%, 92%, 93%, 94%, 95%, 96%, 97%, 98%, 99%, or 100% identity to the amino acid sequence corresponding to positions (i) 15 to 558 of SEQ ID NO:20; or (ii) 15 to 295 of SEQ ID NO:20; the polypeptide has L-α-arabinofuranosidase activity; or

(11) a polypeptide having at least 80%, 85%, 90%, 91%, 92%, 93%, 94%, 95%, 96%, 97%, 98%, 99%, or 100% identity to the amino acid sequence corresponding to positions (i) 21 to 632 of SEQ ID NO:22; (ii) 461 to 632 of SEQ ID NO:22; (iii) 21 to 642 of SEQ ID NO:22; or (iv) 461 to 642 of SEQ ID NO:22; the polypeptide has L-α-arabinofuranosidase activity; or

(12) a polypeptide having at least 80%, 85%, 90%, 91%, 92%, 93%, 94%, 95%, 96%, 97%, 98%, 99%, or 100% identity to the amino acid sequence corresponding to positions (i) 20 to 341 of SEQ ID NO:28; (ii) 21 to 350 of SEQ ID NO:28; (iii) 107 to 341 of SEQ ID NO:28; or (iv) 107 to 350 of SEQ ID NO:28; the polypeptide has β-xylosidase activity; or

(13) a polypeptide having at least 80%, 85%, 90%, 91%, 92%, 93%, 94%, 95%, 96%, 97%, 98%, 99%, or 100% identity to the amino acid sequence corresponding to positions (i) 21 to 660 of SEQ ID NO:32; (ii) 21 to 645 of SEQ ID NO:32; (iii) 450 to 645 of SEQ ID NO:32; or (iv) 450 to 660 of SEQ ID NO:32; the polypeptide has L-α-arabinofuranosidase activity; or

(14) a polypeptide having at least 80%, 85%, 90%, 91%, 92%, 93%, 94%, 95%, 96%, 97%, 98%, 99%, or 100% identity to the amino acid sequence of SEQ ID NO:52, or to residues (i) 22-255, (ii) 22-343, (iii) 307-343, (iv) 307-344, or (v) 22-344 of SEQ ID NO:52; the polypeptide has GH61/endoglucanase activity; or

(15) a polypeptide having at least 80%, 85%, 90%, 91%, 92%, 93%, 94%, 95%, 96%, 97%, 98%, 99%, or 100% identity to the amino acid sequence of SEQ ID NO:54, or to residues (i) 18-282, (ii) 18-601, (iii) 18-733, (iv) 356-601, or (v) 356-733 of SEQ ID NO:54; the polypeptide has β-glucosidase activity; or

(16) a polypeptide having at least 80%, 85%, 90%, 91%, 92%, 93%, 94%, 95%, 96%, 97%, 98%, 99%, or 100% identity to the amino acid sequence of SEQ ID NO:56, or to residues (i) 22-292, (ii) 22-629, (iii) 22-780, (iv) 373-629, or (v) 373-780 of SEQ ID NO:56; the polypeptide has β-glucosidase activity; or

(17) a polypeptide having at least 80%, 85%, 90%, 91%, 92%, 93%, 94%, 95%, 96%, 97%, 98%, 99%, or 100% identity to the amino acid sequence of SEQ ID NO:58, or to residues (i) 20-321, (ii) 20-651, (iii) 20-811, (iv) 423-651, or (v) 423-811 of SEQ ID NO:58; the polypeptide has β-glucosidase activity; or

(18) a polypeptide having at least 80%, 85%, 90%, 91%, 92%, 93%, 94%, 95%, 96%, 97%, 98%, 99%, or 100% identity to the amino acid sequence of SEQ ID NO:60, or to residues (i) 20-327, (ii) 22-600, (iii) 20-899, (iv) 428-899, or (v) 428-660 of SEQ ID NO:60; the polypeptide has β-glucosidase activity; or

(19) a polypeptide having at least 80%, 85%, 90%, 91%, 92%, 93%, 94%, 95%, 96%, 97%, 98%, 99%, or 100% identity to the amino acid sequence of SEQ ID NO:62, or to residues (i) 20-287, (ii) 22-611, (iii) 20-744, (iv) 362-611, or (v) 362-744 of SEQ ID NO:62; the polypeptide has β-glucosidase activity; or

(20) a polypeptide having at least 80%, 85%, 90%, 91%, 92%, 93%, 94%, 95%, 96%, 97%, 98%, 99%, or 100% identity to the amino acid sequence of SEQ ID NO:64, or to residues (i) 19-307, (ii) 19-640, (iii) 19-874, (iv) 407-640, or (v) 407-874 of SEQ ID NO:64; the polypeptide has β-glucosidase activity; or

(21) a polypeptide having at least 80%, 85%, 90%, 91%, 92%, 93%, 94%, 95%, 96%, 97%, 98%, 99%, or 100% identity to the amino acid sequence of SEQ ID NO:66, or to residues (i) 20-297, (ii) 20-629, (iii) 20-857, (iv) 396-629, or (v) 396-857 of SEQ ID NO:66; the polypeptide has β-glucosidase activity; or

(22) a polypeptide having at least 80%, 85%, 90%, 91%, 92%, 93%, 94%, 95%, 96%, 97%, 98%, 99%, or 100% identity to the amino acid sequence of SEQ ID NO:68, or to residues (i) 20-300, (ii) 20-634, (iii) 20-860, (iv) 400-634, or (v) 400-860 of SEQ ID NO:68; the polypeptide has β-glucosidase activity; or

(23) a polypeptide having at least 80%, 85%, 90%, 91%, 92%, 93%, 94%, 95%, 96%, 97%, 98%, 99%, or 100% identity to the amino acid sequence of SEQ ID NO:70, or to residues (i) 20-327, (ii) 20-660, (iii) 20-899, (iv) 428-660, or (v) 428-899 of SEQ ID NO:70; the polypeptide has β-glucosidase activity; or

(24) a polypeptide having at least 80%, 85%, 90%, 91%, 92%, 93%, 94%, 95%, 96%, 97%, 98%, 99%, or 100% identity to the amino acid sequence of SEQ ID NO:72, or to residues (i) 19-314, (ii) 19-647, (iii) 19-886, (iv) 415-647, or (v) 415-886 of SEQ ID NO:72; the polypeptide has β-glucosidase activity; or

(25) a polypeptide having at least 80%, 85%, 90%, 91%, 92%, 93%, 94%, 95%, 96%, 97%, 98%, 99%, or 100% identity to the amino acid sequence of SEQ ID NO:74, or to residues (i) 20-295, (ii) 20-647, (iii) 20-880, (iv) 414-647, or (v) 414-880 of SEQ ID NO:74; the polypeptide has β-glucosidase activity; or

(26) a polypeptide having at least 80%, 85%, 90%, 91%, 92%, 93%, 94%, 95%, 96%, 97%, 98%, 99%, or 100% identity to the amino acid sequence of SEQ ID NO:76, or to residues (i) 19-296, (ii) 19-649, (iii) 19-890, (iv) 415-649, or (v) 415-890 of SEQ ID NO:76; the polypeptide has β-glucosidase activity; or

(27) a polypeptide having at least 80%, 85%, 90%, 91%, 92%, 93%, 94%, 95%, 96%, 97%, 98%, 99%, or 100% identity to the amino acid sequence of SEQ ID NO:78, or to residues (i) 20-354, (ii) 20-660, (iii) 20-805, (iv) 449-660, or (v) 449-805 of SEQ ID NO:78; the polypeptide has β-glucosidase activity; or

(28) a polypeptide having at least 80%, 85%, 90%, 91%, 92%, 93%, 94%, 95%, 96%, 97%, 98%, 99%, or 100% identity to the amino acid sequence of SEQ ID NO:79; the polypeptide has β-glucosidase activity; or

(29) a polypeptide of at least about 100 (e.g., at least about 150, 175, 200, 225, or 250) amino acid residues in length and comprising one or more of the sequence motifs selected from the group consisting of: (1) SEQ ID NOs:84 and 88; (2) SEQ ID NOs:85 and 88; (3) SEQ ID NO:86; (4) SEQ ID NO:87; (5) SEQ ID NOs:84, 88 and 89; (6) SEQ ID NOs:85, 88, and 89; (7) SEQ ID NOs: 84, 88, and 90; (8) SEQ ID NOs: 85, 88 and 90; (9) SEQ ID NOs:84, 88 and 91; (10) SEQ ID NOs: 85, 88 and 91; (11) SEQ ID NOs: 84, 88, 89 and 91; (12) SEQ ID NOs: 84, 88, 90 and 91; (13) SEQ ID NOs: 85, 88, 89 and 91: and (14) SEQ ID NOs: 85, 88, 90 and 91, wherein the polypeptide has GH61/endoglucanase activity; or

(30) a polypeptide comprising at least 2 or more β-glucosidase sequences wherein the first β-glucosidase sequence is at least about 200 (e.g., at least about 200, 220, 240, 260, 280, 300, 320, 340, 360, 380, or 400) residues in length comprising one or more or all of SEQ ID NOs: 197-202, whereas the second β-glucosidase sequence is at least about 50 (e.g., at least about 55, 60, 65, 70, 75, 80, 85, 90, 95, 100, 120, 140, 160, 180, 200) amino acid residues in length and comprising SEQ ID NO:203, wherein the polypeptide optionally also comprises a third β-glucosidase sequence that is about 3, 4, 5, 6, 7, 8, 9, 10, or 11 amino acid residues in length derived from a loop sequence of SEQ ID NOs:66, or comprising an amino acid sequence of FDRRSPG (SEQ ID NO:204), or of FD(R/K)YNIT (SEQ ID NO:205), wherein the polypeptide has β-glucosidase activity.

The present disclosure provides also engineered enzyme compositions (e.g., cellulase compositions) or fermentation broths enriched with one or more of the above-described polypeptides. The cellulase composition can be, e.g., a filamentous fungal cellulase composition, such as a Trichoderma, Chrysosporium, or Aspergillus cellulase composition; a yeast cellulase composition, such as a Saccharomyces cerevisiae cellulase composition, or a bacterial cellulase composition, e.g., a Bacillus cellulase composition. The fermentation broth can be a fermentation broth of a filamentous fungus, for example, a Trichoderma, Humicola, Fusarium, Aspergillus, Neurospora, Penicillium, Cephalosporium, Achlya, Podospora, Endothia, Mucor, Cochliobolus, Pyricularia, or Chrysosporium fermentation broth. In particular, the fermentation broth can be, for example, one of Trichoderma spp. such as a T. reesei, or Penicillium spp., such as a P. funiculosum. The fermentation broth can also suitably be subject to a small set of post-production processing steps, e.g., purification, filtration, ultrafiltration, or a cell-kill step, and then be used in a whole broth formulation.

The disclosure also provides host cells that are recombiantly engineered to express a polypeptide described above. The host cells can be, for example, fungal host cells or bacterial host cells. Fungal host cells can be, e.g., filamentous fungal host cells, such as Trichoderma, Humicola, Fusarium, Aspergillus, Neurospora, Penicillium, Cephalosporium, Achlya, Podospora, Endothia, Mucor, cochliobolus, Pyricularia, or Chrysosporium cells. In particular, the host cells can be, for example, a Trichoderma spp. cell (such as a T. reesei cell), or a Penicillium cell (such as a P. funiculosum cell), an Aspergillus cell (such as an A. oryzae or A. nidulans cell), or a Fusarium cell (such as a F. verticilloides or F. oxysporum cell).

5.1.1 Fusion or Chimeric Proteins

The present disclosure provides a fusion/chimeric protein that includes a domain of a protein of the present disclosure attached to one or more fusion segments, which are typically heterologous to the protein (i.e., derived from a different source than the protein of the disclosure). Suitable fusion/chimeric segments include, without limitation, segments that can enhance a protein's stability, provide other desirable biological activity or enhanced levels of desirable biological activity, and/or facilitate purification of the protein (e.g., by affinity chromatography). A suitable fusion segment can be a domain of any size that has the desired function (e.g., imparts increased stability, solubility, action or biological activity; and/or simplifies purification of a protein). A fuision/hybrid protein can be constructed from 2 or more fusion/chimeric segments, each of which or at least two of which are derived from a different source or microorganism. Fusion/hybrid segments can be joined to amino and/or carboxyl termini of the domain(s) of a protein of the present disclosure. The fusion segments can be susceptible to cleavage. There may be some advantage in having this susceptibility, e.g., it may enable straight-forward recovery of the protein of interest. Fusion proteins are preferably produced by culturing a recombinant cell transfected with a fusion nucleic acid that encodes a protein, which includes a fusion segment attached to either the carboxyl or amino terminal end, or fusion segments attached to both the carboxyl and amino terminal ends, of a protein, or a domain thereof.

In some aspects, the disclosure provides certain chimeric/fusion proteins engineered to comprise 2 or more sequences derived from 2 or more enzymes of different enzyme classes, or 2 or more enzymes of the same or similar classes but derived from different organisms. In certain aspects, the disclosure provides certain chimeric/fusion proteins or polypetpides engineered to improve certain properties such that the chimeric/fusion polypeptides are better suited for desirable industrial applications, for example, when used in hydrolyzing biomass materials. In some aspects, the improved properties can include, for example, improved stability. The improved stability can be reflected an improved proteolytic stability, reflected, e.g., by a lesser degree of proteolytic cleavage observed after a certain period of storage under standard storage conditions, by a lesser degree of proteolytic cleavage observed after the protein is expressed by a host cell during the expression process under suitable expression conditions, or reflected by a lesser degree of proteolytic cleavage observed after the protein is produced recombinantly by the engineered host cell, under, e.g., standard production conditions.

In certain embodiments, the disclosure provides a chimeric/fusion β-glucosidase polypeptide. In some aspects, the chimeric/fusion β-glucosidase comprises 2 or more β-glucosidase sequences, wherein the first sequence is at least about 200 (e.g., at least about 200, 250, 300, 350, or 400) amino acid residues in length and comprises a sequence that has at least about 60% (e.g., at least about 65%, 70%, 75%, 80%, 85%, 90%, 91%, 92%, 93%, 94%, 95%, 96%, 97%, 98%, 99%, or 100%) identity to a sequence of equal length of any one of SEQ ID NOs: 54, 56, 58, 62, 64, 66, 68, 70, 72, 74, 76, 78, and 79, whereas the second sequence is one that is at least about 50 (e.g., at least about 50, 75, 100, 125, 150, or 200) amino acid residues in length and comprises a sequence that has at least about 60% (e.g., at least about 65%, 70%, 75%, 80%, 85%, 90%, 91%, 92%, 93%, 94%, 95%, 96%, 97%, 98%, 99%, or 100%) identity to a sequence of equal length of SEQ ID NO:60. In some aspects, the chimeric/fusion β-glucosidase comprises 2 or more β-glucosidase sequences, wherein the first sequence is at least about 200 (e.g., at least about 200, 250, 300, 350, or 400) amino acid residues in length and comprises a sequence that has at least about 60% (e.g., at least about 65%, 70%, 75%, 80%, 85%, 90%, 91%, 92%, 93%, 94%, 95%, 96%, 97%, 98%, 99%, or 100%) identity to a sequence of equal length of SEQ ID NO:60, whereas the second sequence is one that is at least about 50 (e.g., at least about 50, 75, 100, 125, 150, or 200) amino acid residues in length and comprises a sequence that has at least about 60% (e.g., at least about 65%, 70%, 75%, 80%, 85%, 90%, 91%, 92%, 93%, 94%, 95%, 96%, 97%, 98%, 99%, or 100%) identity to a sequence of equal length of any one of SEQ ID NOs: 54, 56, 58, 62, 64, 66, 68, 70, 72, 74, 76, 78, and 79. In particular, the first of the two or more β-glucosidase sequences is one that is at least about 200 amino acid residues in length and comprises at least 2 (e.g., at least 2, 3, 4, or all) of the amino acid sequence motifs of SEQ ID NOs: 197-202, and the second of the two or more β-glucosidase is at least 50 amino acid residues in length and comprises SEQ ID NO:203. In certain embodiments, the fusion/chimeric β-glucosidase polypeptide has β-glucosidase activity. In some embodiments, the first sequence is located at the N-terminal of the chimeric/fusion β-glucosidase polypeptide, whereas the second sequence is located at the C-terminal of the chimeric/fusion β-glucosidase polypeptide. In some embodiments, the first sequence is connected by its C-terminus to the second sequence by its N-terminus, e.g., the first sequence is immediately adjacent or directly connected to the second sequence. In other embodiments, the first sequence is connected to the second sequence via a linker domain. In certain embodiments, the first sequence, the second sequence, or both the first and the second sequences comprise 1 or more glycosylation sites. In some embodiments, either the first or the second sequence comprises a loop sequence or a sequence that encodes a loop-like structure, derived from a third β-glucosidase polypeptide, which is about 3, 4, 5, 6, 7, 8, 9, 10, or 11 amino acid residues in length, and comprising an amino acid sequence of FDRRSPG (SEQ ID NO:204), or of FD(R/K)YNIT (SEQ ID NO:205). In certain embodiments, neither the first nor the second sequence comprises a loop sequence, rather, the linker domain connecting the first and the second sequences comprise such a loop sequence. In some embodiments, the fusion/chimeric β-glucosidase polypeptide has improved stability as compared to the counterpart β-glucosidase polypeptides from which each of the first, the second, or the linker domain sequences are derived. In some embodiments, the improved stability is an improved proteolytic stability, reflected by a lesser susceptible to proteolytic cleavage at either a residue in the loop sequence or at a residue or position that is outside the loop sequence, to proteolytic cleavage during storage under standard storage conditions, or during expression and/or production under standard expression/production conditions.

In certain aspects, the disclosure provides a fusion/chimeric β-glucosidase polypeptide derived from 2 or more β-glucosidase sequences, wherein the first sequence is derived from Fv3C and is at least about 200 amino acid residues in length, and the second sequence is derived from Tr3B, and is at least about 50 amino acid residues in length. In some embodiments, the C-terminus of the first sequence is connected to the N-terminus of the second sequence, e.g., the first sequence is immediately adjacent or directly connected to the second sequence. In other embodiments, the first sequence is connected to the second sequence via a linker sequence. In some embodiments, either the first or the second sequence comprises a loop sequence, derived from a third β-glucosidase polypeptide, which is about 3, 4, 5, 6, 7, 8, 9, 10, or 11 amino acid residues in length, and comprising an amino acid sequence of FDRRSPG (SEQ ID NO:204), or of FD(R/K)YNIT (SEQ ID NO:205). In certain embodiments, neither the first nor the secone sequence comprises the loop sequence, but rather, the linker sequence connecting the first and the second sequence comprises such a loop sequence. In certain embodiments, the loop sequence is derived from a Te3A polypeptide. In some embodiments, the fusion/chimeric β-glucosidase polypeptide has improved stability as compared to each counterpart β-glucosidase polypeptide from which each of the chimeric parts is derived. For example, the improved stability is over that of the Fv3C polypeptide, the Te3A polypeptide, and/or the Tr3B polypeptide. In some embodiments, the improved stability is an improved proteolytic stability, reflected by, e.g., a lesser susceptibility to proteolytic cleavage at either a residue in the loop sequence or at a residue or position that is outside the loop sequence during storage under standard storage conditions or during expression/production, under standard expression/production conditions. For example, the fusion/chimeric polypeptide is less susceptible to proteolytic cleavage at a residue or position that is to the C-terminal of the loop sequence as compared to an Fv3C polypeptide at the same position when, e.g., the sequences of the chimera and the Fv3C polypeptides are aligned.

Accordingly, proteins of the present disclosure also include expression products of gene fusions (e.g., an overexpressed, soluble, and active form of a recombinant protein), of mutagenized genes (e.g., genes having codon modifications to enhance gene transcription and translation), and of truncated genes (e.g., genes having signal sequences removed or substituted with a heterologous signal sequence).

Glycosyl hydrolases that utilize insoluble substrates are often modular enzymes. They usually comprise catalytic modules appended to 1 or more non-catalytic carbohydrate-binding domains (CBMs). In nature, CBMs are thought to promote the glycosyl hydrolase's interaction with its target substrate polysaccharide. Thus, the disclosure provides chimeric enzymes having altered substrate specificity; including, e.g., chimeric enzymes having multiple substrates as a result of “spliced-in” heterologous CBMs. The heterologous CBMs of the chimeric enzymes of the disclosure can also be designed to be modular, such that they are appended to a catalytic module or catalytic domain (a “CD”, e.g., at an active site), which can be heterologous or homologous to the glycosyl hydrolase. Accordingly the disclosure provides peptides and polypeptides consisting of, or comprising, CBM/CD modules, which can be homologously paired or joined to form chimeric/heterologous CBM/CD pairs. The chimeric polypeptides/peptides can be used to improve or alter the performance of an enzyme of interest.

Accordingly, the disclosure provides chimeric enzymes comprising, e.g., at least one CBM of an enzyme or polypeptide having at least about 60% (e.g., at least about 60%, 65%, 70%, 75%, 80%, 85%, 90%, 91%, 92%, 93%, 94%, 95%, 96%, 97%, 98%, 99%, or 100%) identity to any one of SEQ ID NOs: 54, 56, 58, 60, 62, 64, 66, 68, 70, 72, 74, 76, 78, 79, 93, and 95, over a region of at least about 10 (e.g., at least about 10, 15, 20, 25, 30, 35, 40, 45, 50, 55, 60, 65, 70, 75, 80, 85, 90, 95, 100, 125, 150, 175, 200, 225, 250, 275, 300) residues. In some aspects, the disclosure provides chimeric enzymes comprising, e.g., at least one CBM of an enzyme or polypeptide having at least about 60% (e.g., at least about 60%, 65%, 70%, 75%, 80%, 85%, 90%, 91%, 92%, 93%, 94%, 95%, 96%, 97%, 98%, 99%, or 100%) identity to any one of SEQ ID NOs: 52, 80-81, 206-207, over a region of at least about 10 (e.g., at least about 10, 15, 20, 25, 30, 35, 40, 45, 50, 55, 60, 65, 70, 75, 80, 85, 90, 95, 100, 125, 150, 175, 200, 225, 250, 275, 300) residues. In some aspects, the disclosure provides chimeric enzymes comprising, e.g., at least one CBM of an enzyme or polypeptide having at least about 50 (e.g., at least about 50, 100, 150, 200, 250, or 300) amino acid residues in length, comprising one or more of the sequence motifs selected from the group consisting of (1) SEQ ID NOs:84 and 88; (2) SEQ ID NOs:85 and 88; (3) SEQ ID NO:86; (4) SEQ ID NO:87; (5) SEQ ID NOs:84, 88 and 89; (6) SEQ ID NOs:85, 88, and 89; (7) SEQ ID NOs: 84, 88, and 90; (8) SEQ ID NOs: 85, 88 and 90; (9) SEQ ID NOs:84, 88 and 91; (10) SEQ ID NOs: 85, 88 and 91; (11) SEQ ID NOs: 84, 88, 89 and 91; (12) SEQ ID NOs: 84, 88, 90 and 91; (13) SEQ ID NOs: 85, 88, 89 and 91: and (14) SEQ ID NOs: 85, 88, 90 and 91. In some aspects, the disclosure provides chimeric enzymes comprising, e.g., at least one CBM of an enzyme or polypeptide having at least about 70%, e.g., at least about 71%, 72%, 73%, 74%, 75%, 76%, 77%, 78%, 79%, 80%, 81%, 82%, 83%, 84%, 85%, 86%, 87%, 88%, 89%, 90%, 91%, 92%, 93%, 94%, 95%, 96%, 97%, 98%, or 99%, or complete (100%) identity to a polypeptide of any one of SEQ ID NOs:2, 4, 6, 8, 10, 12, 14, 16, 18, 20, 22, 24, 26, 28, 30, 32, 34, 36, 38, 40, 42, 43, and 45, over a region of at least about 10, e.g., at least about 15, 20, 25, 30, 35, 40, 45, 50, 55, 60, 65, 70, 75, 80, 85, 90, 95, 100, 125, 150, 175, 200, 225, 250, 275, 300, 325, or 350 residues.

The polypeptide of the disclosure can thus suitably be a fusion protein comprising functional domains from two or more different proteins (e.g., a CBM from one protein linked to a CD from another protein).

The polypeptides of the disclosure can suitably be obtained and/or used in “substantially pure” form. For example, a polypeptide of the disclosure constitutes at least about 80 wt. % (e.g., at least about 85 wt. %, 90 wt. %, 91 wt. %, 92 wt. %, 93 wt. %, 94 wt. %, 95 wt. %, 96 wt. %, 97 wt. %, 98 wt. %, or 99 wt. %) of the total protein in a given composition, which also includes other ingredients such as a buffer or solution.

Also, the polypeptides of the disclosure can suitably be obtained and/or used in culture broths (e.g., a filamentous fungal culture broth). The culture broths can be an engineered enzyme composition, for example, the culture broth can be produced by a recombinant host cell that is engineered to express a heterologous polypeptide of the disclosure, or by a recombinant host cell that is engineered to express an endogenous polypeptide of the disclosure in greater or lesser amounts than the endogenous expression levels (e.g., in an amount that is 1-, 2-, 3-, 4-, 5-, or more-fold greater or less than the endogenous expression levels). Furthermore, the culture broths of the invention can be produced by certain “integrated” host cell strains that are engineered to express a plurality of the polypeptides of the disclosure in desired ratios. Exemplary desired ratios are described herein, for example, in Section 5.3 below.

5.2 Nucleic Acids and Host Cells

The present disclosure provides nucleic acids encoding polypeptides of the disclosure, for example those described in Section 5.1 above.

In some aspects, the disclosure provides isolated, synthetic, or recombinant nucleotides encoding a β-glucosidase polypeptide having at least 60% (e.g., at least about 60%, 65%, 70%, 75%, 80%, 85%, 90%, 91%, 92%, 93%, 94%, 95%, 96%, 97%, 98%, 99%, or 100%) sequence identity to any one of SEQ ID NOs: 54, 56, 58, 60, 62, 64, 66, 68, 70, 72, 74, 76, 78, 79, 93, and 95, over a region of at least about 10 (e.g., at least about 10, 15, 20, 25, 30, 35, 40, 45, 50, 55, 60, 65, 70, 75, 80, 85, 90, 95, 100, 125, 150, 175, 200, 225, 250, 275, 300) residues, or over the full length catalytic domain (CD) or the full length carbohydrate binding domain (CBM). In some embodiments, the isolated, synthetic, or recombinant nucleotide encodes a β-glucosidase polypeptide that is a fusion/chimera of two or more β-glucosidase sequences. The fusion/chimeric β-glucosidase polypeptide may comprise a first sequence of at least about 200 (e.g., at least about 200, 250, 300, 350, 400, or 500) amino acid residues in length and may comprise one or more or all of the amino acid sequence motifs of SEQ ID NOs: 96-108. The hybrid/chimeric β-glucosidase polypeptide may comprise a second β-glucosidase sequence that is at least about 50 (e.g., at least about 50, 75, 100, 125, 150, 175, or 200) amino acid residues in length and may comprise one or more or all of the amino acid sequence motifs of SEQ ID NOs: 109-116. In particular, the first of the two or more β-glucosidase sequences is one that is at least about 200 amino acid residues in length and comprises at least 2 (e.g., at least 2, 3, 4, or all) of the amino acid sequence motifs of SEQ ID NOs: 197-202, and the second of the two or more β-glucosidase is at least 50 amino acid residues in length and comprises SEQ ID NO:203. The C-terminus of the first β-glucosidase sequence may be connected to the N-terminus of the second β-glucosidase sequence. In other embodiments, the first and the second β-glucosidase sequences are connected via a linker sequence. The linker sequence may comprise a loop sequence, which is about 3, 4, 5, 6, 7, 8, 9, 10, or 11 amino acid residues in length, derived from a third β-glucosidase polypeptide, and comprises an amino acid sequence of FDRRSPG (SEQ ID NO:204), or of FD(R/K)YNIT (SEQ ID NO:205).

In certain aspects,the disclosure provides an isolated, synthetic, or recombinant nucleotide encoding a β-glucosidase polypeptide, which is a hybrid of at least 2 (e.g., 2, 3, or even 4) β-glucosidase sequences, wherein the first of the at least 2 β-glucosidase sequences is one that is at least about 200 (e.g., at least about 200, 250, 300, 350, or 400) amino acid residues in length and comprises a sequence that has at least about 60% (e.g., at least about 65%, 70%, 75%, 80%, 85%, 90%, 91%, 92%, 93%, 94%, 95%, 96%, 97%, 98%, 99%, or 100%) identity to a sequence of equal length of any one of SEQ ID NOs: 54, 56, 58, 62, 64, 66, 68, 70, 72, 74, 76, 78, and 79, whereas the second of the at least 2 β-glucosidase sequences is one that is at least about 50 (e.g., at least about 50, 75, 100, 125, 150, or 200) amino acid residues in length and comprises a sequence that has at least about 60% (e.g., at least about 65%, 70%, 75%, 80%, 85%, 90%, 91%, 92%, 93%, 94%, 95%, 96%, 97%, 98%, 99%, or 100%) identity to a sequence of equal length of SEQ ID NO:60. In an alternative embodiment, the disclosure provides an isolated, synthetic, or recombinant nucleotide encoding a β-glucosidase polypeptide, which is a hybrid of at least 2 (e.g., 2, 3, or even 4) β-glucosidase sequences, wherein the first of the at least 2 β-glucosidase sequences is one that is at least about 200 (e.g., at least about 200, 250, 300, 350, or 400) amino acid residues in length and comprises a sequence that has at least about 60% (e.g., at least about 65%, 70%, 75%, 80%, 85%, 90%, 91%, 92%, 93%, 94%, 95%, 96%, 97%, 98%, 99%, or 100%) identity to a sequence of equal length of SEQ ID NO:60, whereas the second of the at least 2 β-glucosidase sequences is one that is at least about 50 (e.g., at least about 50, 75, 100, 125, 150, or 200) amino acid residues in length and comprises a sequence that has at least about 60% (e.g., at least about 65%, 70%, 75%, 80%, 85%, 90%, 91%, 92%, 93%, 94%, 95%, 96%, 97%, 98%, 99%, or 100%) identity to a sequence of equal length of any one of SEQ ID NOs: 54, 56, 58, 62, 64, 66, 68, 70, 72, 74, 76, 78, and 79. In certain embodiments, the nucleotide encodes a fusion/chimeric β-glucosidase polypeptide having β-glucosidase activity. In particular, the first of the two or more β-glucosidase sequences is one that is at least about 200 amino acid residues in length and comprises at least 2 (e.g., at least 2, 3, 4, or all) of the amino acid sequence motifs of SEQ ID NOs: 197-202, and the second of the two or more β-glucosidase is at least 50 amino acid residues in length and comprises SEQ ID NO:203. In some embodiments, the nucleotide encodes a first amino acid sequence, which is located at the N-terminal of the chimeric/fusion β-glucosidase polypeptide. In some embodiments, the nucleotide encodes a second amino acid sequence, which is located at the C-terminal of the chimeric/fusion β-glucosidase polypeptide. The C-terminus of the first amino acid sequence may be connected to the N-terminus of the second amino acid sequence. In other embodiments, the first amino acid sequence is not immediately adjacent to the second amino acid sequence, but rather the first sequence is connected to the second sequence via a linker domain. In some embodiments, the first amino acid sequence, the second amino acid sequence or the linker domain comprises an amino acid sequence that comprises a loop sequence, or a sequence that represents a loop-like structure. In certain embodiments, the loop sequence is derived from a third β-glucosidase polypeptide, is about 3, 4, 5, 6, 7, 8, 9, 10, or 11 amino acid residues in length, and comprises an amino acid sequence of FDRRSPG (SEQ ID NO:204), or of FD(R/K)YNIT (SEQ ID NO:205).

In some aspects, the disclosure provides isolated, synthetic, or recombinant nucleotides having at least 60% (e.g., at least about 60%, 65%, 70%, 75%, 80%, 85%, 90%, 91%, 92%, 93%, 94%, 95%, 96%, 97%, 98%, 99%, or 100%) sequence identity to any one of SEQ ID NOs: 52, 55, 57, 59, 61, 63, 65, 67, 69, 71, 73, 75, 77, 92 or 94, or to a fragment of at least about 300 (e.g., at least about 300, 400, 500, or 600) residues in length of any one of SEQ ID NOs: 53, 55, 57, 59, 61, 63, 65, 67, 69, 71, 73, 75, 77, 92 or 94. In certain embodiments, the disclosure provides isolated, synthetic, or recombinant nucleotides that are capable of hybridizing to any one of SEQ ID NOs: 53, 55, 57, 59, 61, 63, 65, 67, 69, 71, 73, 75, 77, 92 or 94, to a fragment of at least about 300 residues in length, or to a complement thereof, under low stringency, medium stringency, high stringency, or very high stringency conditions.

In some aspects, the disclosure provides an isolated, synthetic, or recombinant nucleotide encoding a polypeptide comprising an amino acid sequence having at least about 60% (e.g., at least about 60%, 65%, 70%, 75%, 80%, 85%, 90%, 91%, 92%, 93%, 94%, 95%, 96%, 97%, 98%, 99%, or 100%) sequence identity to any one of SEQ ID NOs: 52, 80-81, 206-207, over a region of at least about 10 (e.g., at least about 10, 15, 20, 25, 30, 35, 40, 45, 50, 55, 60, 65, 70, 75, 80, 85, 90, 95, 100, 125, 150, 175, 200, 225, 250, 275, 300) residues, or over the full length catalytic domain (CD) or the full length carbohydrate binding domain (CBM). In certain embodiments, the isolated, synthetic, or recombiant nucleotide encodes a polypeptide have GH61/endoglucanase activity. In some embodiments, the disclosure provides an isolated, synthetic or recombinant encoding a polypeptide comprising an amino acid sequence of at least about 50 (e.g., at least about 50, 100, 150, 200, 250, or 300) amino acid residues in length, comprising one or more of the sequence motifs selected from the group consisting of (1) SEQ ID NOs:84 and 88; (2) SEQ ID NOs:85 and 88; (3) SEQ ID NO:86; (4) SEQ ID NO:87; (5) SEQ ID NOs:84, 88 and 89; (6) SEQ ID NOs:85, 88, and 89; (7) SEQ ID NOs: 84, 88, and 90; (8) SEQ ID NOs: 85, 88 and 90; (9) SEQ ID NOs:84, 88 and 91; (10) SEQ ID NOs: 85, 88 and 91; (11) SEQ ID NOs: 84, 88, 89 and 91; (12) SEQ ID NOs: 84, 88, 90 and 91; (13) SEQ ID NOs: 85, 88, 89 and 91: and (14) SEQ ID NOs: 85, 88, 90 and 91. In certain embodiments, the polynucleotide is one that encodes a polypeptide having at least about 60%, 65%, 70%, 75%, 80%, 85%, 90%, 91%, 92%, 93%, 94%, 95%, 96%, 97%, 98%, 99%, or 100% sequence identity to SEQ ID NO:52. In some embodiments, the polynucleotide encodes a GH61 endoglucanase polypeptide (e.g., an EG IV polypeptide from a suitable organism, such as, without limitation, T. reesei Eg4).

In some aspects, the disclosure provides an isolated, synthetic, or recombinant polynucleotide encoding a polypeptide having at least about 70%, (e.g., at least about 71%, 72%, 73%, 74%, 75%, 76%, 77%, 78%, 79%, 80%, 81%, 82%, 83%, 84%, 85%, 86%, 87%, 88%, 89%, 90%, 91%, 92%, 93%, 94%, 95%, 96%, 97%, 98%, or 99%, or complete (100%)) sequence identity to a polypeptide of any one of SEQ ID NOs:2, 4, 6, 8, 10, 12, 14, 16, 18, 20, 22, 24, 26, 28, 30, 32, 34, 36, 38, 40, 42, 43, and 45, over a region of at least about 10, e.g., at least about 15, 20, 25, 30, 35, 40, 45, 50, 55, 60, 65, 70, 75, 80, 85, 90, 95, 100, 125, 150, 175, 200, 225, 250, 275, 300, 325, or 350 residues, or over the full length immature polypeptide, the full length mature polypeptide, the full length catalytic domain (CD) or the full length carbohydrate binding domain (CBM). In some aspects, the disclosure provides an isolated, synthetic, or recombinant polynucleotide having at least about 70% (e.g., at least about 71%, 72%, 73%, 74%, 75%, 76%, 77%, 78%, 79%, 80%, 81%, 82%, 83%, 84%, 85%, 86%, 87%, 88%, 89%, 90%, 91%, 92%, 93%, 94%, 95%, 96%, 97%, 98%, or 99%, or complete (100%)) sequence identity to any one of SEQ ID NOs: 1, 3, 5, 7, 9, 11, 13, 15, 17, 19, 21, 23, 25, 27, 29, 31, 33, 35, 37, 39, and 41, or to a fragment thereof. For example, the fragment may be at least about 10, 20, 30, 40, 50, 60, 70, 80, 90, 100 residues in length. In some embodiments, the disclosure provides an isolated, synthetic, or recombinant polynucleotide that hybridizes under low stringency conditions, medium stringency conditions, high stringency conditions, or very high stringency conditions to any one of SEQ ID NOs: 1, 3, 5, 7, 9, 11, 13, 15, 17, 19, 21, 23, 25, 27, 29, 31, 33, 35, 37, 39, and 41, or to a fragment or subsequence thereof.

The disclosure thus specifically provides a nucleic acid encoding Fv3A, Pf43A, Fv43E, Fv39A, Fv43A, Fv43B, Pa51A, Gz43A, Fo43A, Af43A, Pf51A, AfuXyn2, AfuXyn5, Fv43D, Pf43B, Fv43B, Fv51A, T. reesei Xyn3, T. reesei Xyn2, T. reesei Bxl1, T. reesei Eg4, Pa3D, Fv3G, Fv3D, Fv3C, Tr3A, Tr3B, Te3A, An3A, Fo3A, Gz3A, Nh3A, Vd3A, Pa3G or a Tn3B polypeptide (including a variant, mutant, or fusion/chimera thereof). The disclosure further provides a nucleic acid encoding a chimeric or fusion enzyme comprising a part of Fv3C and a part of Tr3B. The chimeric or fusion polypeptide, in some embodiments, can further comprise a linker domain comprising a loop sequence of at least about 3, 4, 5, 6, 7, 8, 9, 10, or 11 amino acid residues derived from Te3A. For example, the disclosure provides an isolated nucleotide having at least about 60% sequence identity to 92 or 94.

For example, the disclosure provides an isolated nucleic acid molecule, wherein the nucleic acid molecule encodes:

(1) a polypeptide comprising an amino acid sequence with at least 80%, 85%, 90%, 91%, 92%, 93%, 94%, 95%, 96%, 97%, 98%, 99%, or 100% sequence identity to the amino acid sequence corresponding to positions (i) 24 to 766 of SEQ ID NO:2; (ii) 73 to 321 of SEQ ID NO:2; (iii) 73 to 394 of SEQ ID NO:2; (iv) 395 to 622 of SEQ ID NO:2; (v) 24 to 622 of SEQ ID NO:2; or (iv) 73 to 622 of SEQ ID NO:2; the polypeptide preferably has β-xylosidase activity; or

(2) a polypeptide comprising an amino acid sequence with at least 80%, 85%, 90%, 91%, 92%, 93%, 94%, 95%, 96%, 97%, 98%, 99%, or 100% sequence identity to the amino acid sequence corresponding to positions (i) 21 to 445 of SEQ ID NO:4; (ii) 21 to 301 of SEQ ID NO:4; (iii) 21 to 323 of SEQ ID NO:4; (iv) 21 to 444 of SEQ ID NO:4; (v) 302 to 444 of SEQ ID NO:4; (vi) 302 to 445 of SEQ ID NO:4; (vii) 324 to 444 of SEQ ID NO:4; or (viii) 324 to 445 of SEQ ID NO:4; the polypeptide preferably has β-xylosidase activity; or

(3) a polypeptide comprising an amino acid sequence with at least 80%, 85%, 90%, 91%, 92%, 93%, 94%, 95%, 96%, 97%, 98%, 99%, or 100% sequence identity to the amino acid sequence corresponding to positions (i) 19 to 530 of SEQ ID NO:6; (ii) 29 to 530 of SEQ ID NO:6; (iii) 19 to 300 of SEQ ID NO:6; or (iv) 29 to 300 of SEQ ID NO:6; the polypeptide preferably has β-xylosidase activity; or

(4) a polypeptide comprising an amino acid sequence with at least 80%, 85%, 90%, 91%, 92%, 93%, 94%, 95%, 96%, 97%, 98%, 99%, or 100% sequence identity to the amino acid sequence corresponding to positions (i) 20 to 439 of SEQ ID NO:8; (ii) 20 to 291 of SEQ ID NO:8; (iii) 145 to 291 of SEQ ID NO:8; or (iv) 145 to 439 of SEQ ID NO:8; the polypeptide preferably has β-xylosidase activity; or

(5) a polypeptide comprising an amino acid sequence with at least 80%, 85%, 90%, 91%, 92%, 93%, 94%, 95%, 96%, 97%, 98%, 99%, or 100% sequence identity to the amino acid sequence corresponding to positions (i) 23 to 449 of SEQ ID NO:10; (ii) 23 to 302 of SEQ ID NO:10; (iii) 23 to 320 of SEQ ID NO:10; (iv) 23 to 448 of SEQ ID NO:10; (v) 303 to 448 of SEQ ID NO:10; (vi) 303 to 449 of SEQ ID NO:10; (vii) 321 to 448 of SEQ ID NO:10; or (viii) 321 to 449 of SEQ ID NO:10; the polypeptide preferably has β-xylosidase activity; or

(6) a polypeptide comprising an amino acid sequence with at least 80%, 85%, 90%, 91%, 92%, 93%, 94%, 95%, 96%, 97%, 98%, 99%, or 100% sequence identity to the amino acid sequence corresponding to positions (i) 17 to 574 of SEQ ID NO:12; (ii) 27 to 574 of SEQ ID NO:12; (iii) 17 to 303 of SEQ ID NO:12; or (iv) 27 to 303 of SEQ ID NO:12; the polypeptide preferably has both β-xylosidase activity and L-α-arabinofuranosidase activity; or

(7) a polypeptide comprising an amino acid sequence with at least 80%, 85%, 90%, 91%, 92%, 93%, 94%, 95%, 96%, 97%, 98%, 99%, or 100% sequence identity to the amino acid sequence corresponding to positions (i) 21 to 676 of SEQ ID NO:14; (ii) 21 to 652 of SEQ ID NO:14; (iii) 469 to 652 of SEQ ID NO:14; or (iv) 469 to 676 of SEQ ID NO:14; the polypeptide preferably has β-xylosidase activity and L-α-arabinofuranosidase activity; or

(8) a polypeptide comprising an amino acid sequence with at least 80%, 85%, 90%, 91%, 92%, 93%, 94%, 95%, 96%, 97%, 98%, 99%, or 100% sequence identity to the amino acid sequence corresponding to positions (i) 19 to 340 of SEQ ID NO:16; (ii) 53 to 340 of SEQ ID NO:16; (iii) 19 to 383 of SEQ ID NO:16; or (iv) 53 to 383 of SEQ ID NO:16; the polypeptide preferably has β-xylosidase activity; or

(9) a polypeptide comprising an amino acid sequence with at least 80%, 85%, 90%, 91%, 92%, 93%, 94%, 95%, 96%, 97%, 98%, 99%, or 100% sequence identity to the amino acid sequence corresponding to positions (i) 21 to 341 of SEQ ID NO:18; (ii) 107 to 341 of SEQ ID NO:18; (iii) 21 to 348 of SEQ ID NO:18; or (iv) 107 to 348 of SEQ ID NO:18; the polypeptide preferably has β-xylosidase activity; or

(10) a polypeptide comprising an amino acid sequence with at least 80%, 85%, 90%, 91%, 92%, 93%, 94%, 95%, 96%, 97%, 98%, 99%, or 100% sequence identity to the amino acid sequence corresponding to positions (i) 15 to 558 of SEQ ID NO:20; or (ii) 15 to 295 of SEQ ID NO:20; the polypeptide preferably has L-α-arabinofuranosidase activity; or

(11) a polypeptide comprising an amino acid sequence with at least 80%, 85%, 90%, 91%, 92%, 93%, 94%, 95%, 96%, 97%, 98%, 99%, or 100% sequence identity to the amino acid sequence corresponding to positions (i) 21 to 632 of SEQ ID NO:22; (ii) 461 to 632 of SEQ ID NO:22; (iii) 21 to 642 of SEQ ID NO:22; or (iv) 461 to 642 of SEQ ID NO:22; the polypeptide preferably has L-α-arabinofuranosidase activity; or

(12) a polypeptide comprising an amino acid sequence with at least 80%, 85%, 90%, 91%, 92%, 93%, 94%, 95%, 96%, 97%, 98%, 99%, or 100% sequence identity to the amino acid sequence corresponding to positions (i) 20 to 341 of SEQ ID NO:28; (ii) 21 to 350 of SEQ ID NO:28; (iii) 107 to 341 of SEQ ID NO:28; or (iv) 107 to 350 of SEQ ID NO:28; the polypeptide has β-xylosidase activity; or

(13) a polypeptide comprising an amino acid sequence with at least 80%, 85%, 90%, 91%, 92%, 93%, 94%, 95%, 96%, 97%, 98%, 99%, or 100% sequence identity to the amino acid sequence corresponding to positions (i) 21 to 660 of SEQ ID NO:32; (ii) 21 to 645 of SEQ ID NO:32; (iii) 450 to 645 of SEQ ID NO:32; or (iv) 450 to 660 of SEQ ID NO:32; the polypeptide preferably has L-α-arabinofuranosidase activity; or

(14) a polypeptide comprising an amino acid sequence with at least 80%, 85%, 90%, 91%, 92%, 93%, 94%, 95%, 96%, 97%, 98%, 99%, or 100% sequence identity to the amino acid sequence of SEQ ID NO:52, or to residues (i) 22-255, (ii) 22-343, (iii) 307-343, (iv) 307-344, or (v) 22-344 of SEQ ID NO:52; the polypeptide preferably has GH61/endoglucanase activity; or

(15) a polypeptide comprising an amino acid sequence with at least 80%, 85%, 90%, 91%, 92%, 93%, 94%, 95%, 96%, 97%, 98%, 99%, or 100% sequence identity to the amino acid sequence of SEQ ID NO:54, or to residues (i) 18-282, (ii) 18-601, (iii) 18-733, (iv) 356-601, or (v) 356-733 of SEQ ID NO:54; the polypeptide preferably has β-glucosidase activity; or

(16) a polypeptide comprising an amino acid sequence with at least 80%, 85%, 90%, 91%, 92%, 93%, 94%, 95%, 96%, 97%, 98%, 99%, or 100% sequence identity to the amino acid sequence of SEQ ID NO:56, or to residues (i) 22-292, (ii) 22-629, (iii) 22-780, (iv) 373-629, or (v) 373-780 of SEQ ID NO:56; the polypeptide preferably has β-glucosidase activity; or

(17) a polypeptide comprising an amino acid sequence with at least 80%, 85%, 90%, 91%, 92%, 93%, 94%, 95%, 96%, 97%, 98%, 99%, or 100% sequence identity to the amino acid sequence of SEQ ID NO:58, or to residues (i) 20-321, (ii) 20-651, (iii) 20-811, (iv) 423-651, or (v) 423-811 of SEQ ID NO:58; the polypeptide preferably has β-glucosidase activity; or

(18) a polypeptide comprising an amino acid sequence with at least 80%, 85%, 90%, 91%, 92%, 93%, 94%, 95%, 96%, 97%, 98%, 99%, or 100% sequence identity to the amino acid sequence of SEQ ID NO:60, or to residues (i) 20-327, (ii) 22-600, (iii) 20-899, (iv) 428-899, or (v) 428-660 of SEQ ID NO:60; the polypeptide preferably has β-glucosidase activity; or

(19) a polypeptide comprising an amino acid sequence with at least 80%, 85%, 90%, 91%, 92%, 93%, 94%, 95%, 96%, 97%, 98%, 99%, or 100% sequence identity to the amino acid sequence of SEQ ID NO:62, or to residues (i) 20-287, (ii) 22-611, (iii) 20-744, (iv) 362-611, or (v) 362-744 of SEQ ID NO:62; the polypeptide preferably has β-glucosidase activity; or

(20) a polypeptide comprising an amino acid sequence with at least 80%, 85%, 90%, 91%, 92%, 93%, 94%, 95%, 96%, 97%, 98%, 99%, or 100% sequence identity to the amino acid sequence of SEQ ID NO:64, or to residues (i) 19-307, (ii) 19-640, (iii) 19-874, (iv) 407-640, or (v) 407-874 of SEQ ID NO:64; the polypeptide preferably has β-glucosidase activity; or

(21) a polypeptide comprising an amino acid sequence with at least 80%, 85%, 90%, 91%, 92%, 93%, 94%, 95%, 96%, 97%, 98%, 99%, or 100% sequence identity to the amino acid sequence of SEQ ID NO:66, or to residues (i) 20-297, (ii) 20-629, (iii) 20-857, (iv) 396-629, or (v) 396-857 of SEQ ID NO:66; the polypeptide preferably has β-glucosidase activity; or

(22) a polypeptide comprising an amino acid sequence with at least 80%, 85%, 90%, 91%, 92%, 93%, 94%, 95%, 96%, 97%, 98%, 99%, or 100% sequence identity to the amino acid sequence of SEQ ID NO:68, or to residues (i) 20-300, (ii) 20-634, (iii) 20-860, (iv) 400-634, or (v) 400-860 of SEQ ID NO:68; the polypeptide preferably has β-glucosidase activity; or

(23) a polypeptide comprising an amino acid sequence with at least 80%, 85%, 90%, 91%, 92%, 93%, 94%, 95%, 96%, 97%, 98%, 99%, or 100% sequence identity to the amino acid sequence of SEQ ID NO:70, or to residues (i) 20-327, (ii) 20-660, (iii) 20-899, (iv) 428-660, or (v) 428-899 of SEQ ID NO:70; the polypeptide preferably has β-glucosidase activity; or

(24) a polypeptide comprising an amino acid sequence with at least 80%, 85%, 90%, 91%, 92%, 93%, 94%, 95%, 96%, 97%, 98%, 99%, or 100% sequence identity to the amino acid sequence of SEQ ID NO:72, or to residues (i) 19-314, (ii) 19-647, (iii) 19-886, (iv) 415-647, or (v) 415-886 of SEQ ID NO:72; the polypeptide preferably has β-glucosidase activity; or

(25) a polypeptide comprising an amino acid sequence with at least 80%, 85%, 90%, 91%, 92%, 93%, 94%, 95%, 96%, 97%, 98%, 99%, or 100% sequence identity to the amino acid sequence of SEQ ID NO:74, or to residues (i) 20-295, (ii) 20-647, (iii) 20-880, (iv) 414-647, or (v) 414-880 of SEQ ID NO:74; the polypeptide preferably has β-glucosidase activity; or

(26) a polypeptide comprising an amino acid sequence with at least 80%, 85%, 90%, 91%, 92%, 93%, 94%, 95%, 96%, 97%, 98%, 99%, or 100% sequence identity to the amino acid sequence of SEQ ID NO:76, or to residues (i) 19-296, (ii) 19-649, (iii) 19-890, (iv) 415-649, or (v) 415-890 of SEQ ID NO:76; the polypeptide preferably has β-glucosidase activity; or

(27) a polypeptide comprising an amino acid sequence with at least 80%, 85%, 90%, 91%, 92%, 93%, 94%, 95%, 96%, 97%, 98%, 99%, or 100% sequence identity to the amino acid sequence of SEQ ID NO:78, or to residues (i) 20-354, (ii) 20-660, (iii) 20-805, (iv) 449-660, or (v) 449-805 of SEQ ID NO:78; the polypeptide preferably has β-glucosidase activity; or

(28) a polypeptide comprising an amino acid sequence with at least 80%, 85%, 90%, 91%, 92%, 93%, 94%, 95%, 96%, 97%, 98%, 99%, or 100% sequence identity to the amino acid sequence of SEQ ID NO:79; the polypeptide preferably has β-glucosidase activity; or

(29) a polypeptide of at least about 100 (e.g., at least about 150, 175, 200, 225, or 250) residues in length and comprising one or more of the sequence motifs selected from the group consisting of: (1) SEQ ID NOs:84 and 88; (2) SEQ ID NOs:85 and 88; (3) SEQ ID NO:86; (4) SEQ ID NO:87; (5) SEQ ID NOs:84, 88 and 89; (6) SEQ ID NOs:85, 88, and 89; (7) SEQ ID NOs: 84, 88, and 90; (8) SEQ ID NOs: 85, 88 and 90; (9) SEQ ID NOs:84, 88 and 91; (10) SEQ ID NOs: 85, 88 and 91; (11) SEQ ID NOs: 84, 88, 89 and 91; (12) SEQ ID NOs: 84, 88, 90 and 91; (13) SEQ ID NOs: 85, 88, 89 and 91: and (14) SEQ ID NOs: 85, 88, 90 and 91, wherein the polypeptide preferably has GH61/endoglucanase activity; or

(30) a polypeptide comprising at least two or more β-glucosidase sequences wherein the first β-glucosidase sequence is at least about 200 (e.g., at least about 200, 220, 240, 260, 280, 300, 320, 340, 360, 380, or 400) residues in length comprising one or more or all of SEQ ID NOs: 96-108, whereas the second β-glucosidase sequence is at least about 50 (e.g., at least about 55, 60, 65, 70, 75, 80, 85, 90, 95, 100, 120, 140, 160, 180, 200) amino acid residues in length and comprising one or more or all of SEQ ID NOs:109-116, wherein the polypeptide optionally also comprises a third β-glucosidase sequence that is about 3, 4, 5, 6, 7, 8, 9, 10, or 11 amino acid residues in length derived from a loop sequence of SEQ ID NOs:66, wherein the polypeptide preferably has β-glucosidase activity.

The instant disclosure also provides:

(1) a nucleic acid having at least 80% (e.g., at least 80%, 85%, 90%, 91%, 92%, 93%, 94%, 95%, 96%, 97%, 98%, 99% or more) sequence identity to SEQ ID NO:1, or a nucleic acid that is capable of hybridizing under high stringency conditions to a complement of SEQ ID NO:1, or to a fragment thereof; or

(2) a nucleic acid having at least 80% (e.g., at least 80%, 85%, 90%, 91%, 92%, 93%, 94%, 95%, 96%, 97%, 98%, 99% or more)sequence identity to SEQ ID NO:3, or a nucleic acid that is capable of hybridizing under high stringency conditions to a complement of SEQ ID NO:3, or to a fragment thereof; or

(3) a nucleic acid having at least 80% (e.g., at least 80%, 85%, 90%, 91%, 92%, 93%, 94%, 95%, 96%, 97%, 98%, 99% or more) sequence identity to SEQ ID NO:5, or a nucleic acid that is capable of hybridizing under high stringency conditions to a complement of SEQ ID NO:5, or to a fragment thereof; or

(4) a nucleic acid having at least 80% (e.g., at least 80%, 85%, 90%, 91%, 92%, 93%, 94%, 95%, 96%, 97%, 98%, 99% or more) sequence identity to SEQ ID NO:7, or a nucleic acid that is capable of hybridizing under high stringency conditions to a complement of SEQ ID NO:7, or to a fragment thereof; or

(5) a nucleic acid having at least 80% (e.g., at least 80%, 85%, 90%, 91%, 92%, 93%, 94%, 95%, 96%, 97%, 98%, 99% or more) sequence identity to SEQ ID NO:9, or a nucleic acid that is capable of hybridizing under high stringency conditions to a complement of SEQ ID NO:9, or to a fragment thereof; or

(6) a nucleic acid having at least 80% (e.g., at least 80%, 85%, 90%, 91%, 92%, 93%, 94%, 95%, 96%, 97%, 98%, 99% or more) sequence identity to SEQ ID NO:11, or a nucleic acid that is capable of hybridizing under high stringency conditions to a complement of SEQ ID NO:11, or to a fragment thereof; or

(7) a nucleic acid having at least 80% (e.g., at least 80%, 85%, 90%, 91%, 92%, 93%, 94%, 95%, 96%, 97%, 98%, 99% or more) sequence identity to SEQ ID NO:13, or a nucleic acid that is capable of hybridizing under high stringency conditions to a complement of SEQ ID NO:13, or to a fragment thereof; or

(8) a nucleic acid having at least 80% (e.g., at least 80%, 85%, 90%, 91%, 92%, 93%, 94%, 95%, 96%, 97%, 98%, 99% or more) sequence identity to SEQ ID NO:15, or a nucleic acid that is capable of hybridizing under high stringency conditions to a complement of SEQ ID NO:15, or to a fragment thereof; or

(9) a nucleic acid having at least 80% (e.g., at least 80%, 85%, 90%, 91%, 92%, 93%, 94%, 95%, 96%, 97%, 98%, 99% or more) sequence identity to SEQ ID NO:17, or a nucleic acid that is capable of hybridizing under high stringency conditions to a complement of SEQ ID NO:17, or to a fragment thereof; or

(10) a nucleic acid having at least 80% (e.g., at least 80%, 85%, 90%, 91%, 92%, 93%, 94%, 95%, 96%, 97%, 98%, 99% or more) sequence identity to SEQ ID NO:19, or a nucleic acid that is capable of hybridizing under high stringency conditions to a complement of SEQ ID NO:19, or to a fragment thereof; or

(11) a nucleic acid having at least 80% (e.g., at least 80%, 85%, 90%, 91%, 92%, 93%, 94%, 95%, 96%, 97%, 98%, 99% or more) sequence identity to SEQ ID NO:21, or a nucleic acid that is capable of hybridizing under high stringency conditions to a complement of SEQ ID NO:21, or to a fragment thereof; or

(12) a nucleic acid having at least 80% (e.g., at least 80%, 85%, 90%, 91%, 92%, 93%, 94%, 95%, 96%, 97%, 98%, 99% or more) sequence identity to SEQ ID NO:27, or a nucleic acid that is capable of hybridizing under high stringency conditions to a complement of SEQ ID NO:27, or to a fragment thereof; or

(13) a nucleic acid having at least 80% (e.g., at least 80%, 85%, 90%, 91%, 92%, 93%, 94%, 95%, 96%, 97%, 98%, 99% or more) sequence identity to SEQ ID NO:31, or a nucleic acid that is capable of hybridizing under high stringency conditions to a complement of SEQ ID NO:31, or to a fragment thereof; or

(14) a nucleic acid having at least 80% (e.g., at least 80%, 85%, 90%, 91%, 92%, 93%, 94%, 95%, 96%, 97%, 98%, 99% or more) sequence identity to SEQ ID NO:51, or a nucleic acid that is capable of hybridizing under high stringency conditions to a complement of SEQ ID NO:51, or to a fragment thereof; or

(15) a nucleic acid having at least 80% (e.g., at least 80%, 85%, 90%, 91%, 92%, 93%, 94%, 95%, 96%, 97%, 98%, 99% or more) sequence identity to SEQ ID NO:53, or a nucleic acid that is capable of hybridizing under high stringency conditions to a complement of SEQ ID NO:53, or to a fragment thereof; or

(16) a nucleic acid having at least 80% (e.g., at least 80%, 85%, 90%, 91%, 92%, 93%, 94%, 95%, 96%, 97%, 98%, 99% or more) sequence identity to SEQ ID NO:55, or a nucleic acid that is capable of hybridizing under high stringency conditions to a complement of SEQ ID NO:55, or to a fragment thereof; or

(17) a nucleic acid having at least 80% (e.g., at least 80%, 85%, 90%, 91%, 92%, 93%, 94%, 95%, 96%, 97%, 98%, 99% or more) sequence identity to SEQ ID NO:57, or a nucleic acid that is capable of hybridizing under high stringency conditions to a complement of SEQ ID NO:57, or to a fragment thereof; or

(18) a nucleic acid having at least 80% (e.g., at least 80%, 85%, 90%, 91%, 92%, 93%, 94%, 95%, 96%, 97%, 98%, 99% or more) sequence identity to SEQ ID NO:59, or a nucleic acid that is capable of hybridizing under high stringency conditions to a complement of SEQ ID NO:59, or to a fragment thereof; or

(19) a nucleic acid having at least 80% (e.g., at least 80%, 85%, 90%, 91%, 92%, 93%, 94%, 95%, 96%, 97%, 98%, 99% or more) sequence identity to SEQ ID NO:61, or a nucleic acid that is capable of hybridizing under high stringency conditions to a complement of SEQ ID NO:61, or to a fragment thereof; or

(20) a nucleic acid having at least 80% (e.g., at least 80%, 85%, 90%, 91%, 92%, 93%, 94%, 95%, 96%, 97%, 98%, 99% or more) sequence identity to SEQ ID NO:63, or a nucleic acid that is capable of hybridizing under high stringency conditions to a complement of SEQ ID NO:63, or to a fragment thereof; or

(21) a nucleic acid having at least 80% (e.g., at least 80%, 85%, 90%, 91%, 92%, 93%, 94%, 95%, 96%, 97%, 98%, 99% or more) sequence identity to SEQ ID NO:65, or a nucleic acid that is capable of hybridizing under high stringency conditions to a complement of SEQ ID NO:65, or to a fragment thereof; or

(22) a nucleic acid having at least 80% (e.g., at least 80%, 85%, 90%, 91%, 92%, 93%, 94%, 95%, 96%, 97%, 98%, 99% or more) sequence identity to SEQ ID NO:67, or a nucleic acid that is capable of hybridizing under high stringency conditions to a complement of SEQ ID NO:67, or to a fragment thereof; or

(23) a nucleic acid having at least 80% (e.g., at least 80%, 85%, 90%, 91%, 92%, 93%, 94%, 95%, 96%, 97%, 98%, 99% or more) sequence identity to SEQ ID NO:69, or a nucleic acid that is capable of hybridizing under high stringency conditions to a complement of SEQ ID NO:69, or to a fragment thereof; or

(24) a nucleic acid having at least 80% (e.g., at least 80%, 85%, 90%, 91%, 92%, 93%, 94%, 95%, 96%, 97%, 98%, 99% or more) sequence identity to SEQ ID NO:71, or a nucleic acid that is capable of hybridizing under high stringency conditions to a complement of SEQ ID NO:71, or to a fragment thereof; or

(25) a nucleic acid having at least 80% (e.g., at least 80%, 85%, 90%, 91%, 92%, 93%, 94%, 95%, 96%, 97%, 98%, 99% or more) sequence identity to SEQ ID NO:73, or a nucleic acid that is capable of hybridizing under high stringency conditions to a complement of SEQ ID NO:73, or to a fragment thereof; or

(26) a nucleic acid having at least 80% (e.g., at least 80%, 85%, 90%, 91%, 92%, 93%, 94%, 95%, 96%, 97%, 98%, 99% or more) sequence identity to SEQ ID NO:75, or a nucleic acid that is capable of hybridizing under high stringency conditions to a complement of SEQ ID NO:75, or to a fragment thereof; or

(27) a nucleic acid having at least 80% (e.g., at least 80%, 85%, 90%, 91%, 92%, 93%, 94%, 95%, 96%, 97%, 98%, 99% or more) sequence identity to SEQ ID NO:77, or a nucleic acid that is capable of hybridizing under high stringency conditions to a complement of SEQ ID NO:77, or to a fragment thereof; or

(28) a nucleic acid having at least 80% (e.g., at least 80%, 85%, 90%, 91%, 92%, 93%, 94%, 95%, 96%, 97%, 98%, 99% or more) sequence identity to SEQ ID NO:92, or a nucleic acid that is capable of hybridizing under high stringency conditions to a complement of SEQ ID NO:92, or to a fragment thereof; or

(29) a nucleic acid having at least 80% (e.g., at least 80%, 85%, 90%, 91%, 92%, 93%, 94%, 95%, 96%, 97%, 98%, 99% or more) sequence identity to SEQ ID NO:94, or a nucleic acid that is capable of hybridizing under high stringency conditions to a complement of SEQ ID NO:94, or to a fragment thereof.

The disclosure also provides expression cassettes and/or vectors comprising the above-described nucleic acids. Suitably, the nucleic acid encoding an enzyme of the disclosure is operably linked to a promoter. Specifically, where recombinant expression in a filamentous fungal host is desired, the promoter can be a filamentous fungal promoter. The nucleic acids may be under the control of heterologous promoters. The nucleic acids may also be expressed under the control of constitutive or inducible promoters. Examples of promoters that can be used include, without limitation, a cellulase promoter, a xylanase promoter, the 1818 promoter (previously identified as a highly expressed protein by EST mapping Trichoderma). For example, the promoter may be a cellobiohydrolase, endoglucanase, or β-glucosidase promoter. A particulary suitable promoter may be, e.g., a T. reesei cellobiohydrolase, endoglucanase, or β-glucosidase promoter. For example, the promoter is a cellobiohydrolase I (cbh1) promoter. Non-limiting examples of promoters include a cbh1, cbh2, egl1, egl2, egl3, egl4, egl5, pki1, gpd1, xyn1, or xyn2 promoter. Additional non-limiting examples of promoters include a T. reesei cbh1, cbh2, egl1, egl2, egl3, egl4, egl5, pki1, gpd1, xyn1, or xyn2 promoter.

As used herein, the term “operably linked” means that selected nucleotide sequence (e.g., encoding a polypeptide described herein) is in proximity with a promoter to allow the promoter to regulate expression of the selected DNA. In addition, the promoter is located upstream of the selected nucleotide sequence in terms of the direction of transcription and translation. The nucleotide sequence and a regulatory sequence(s) are connected in such a way as to permit gene expression when the appropriate molecules (e.g., transcriptional activator proteins) are bound to the regulatory sequence(s).

The present disclosure provides host cells that are engineered to express one or more enzymes of the disclosure. Suitable host cells include cells of any microorganism (e.g., cells of a bacterium, a protist, an alga, a fungus (e.g., a yeast or filamentous fungus), or other microbe), and are preferably cells of a bacterium, a yeast, or a filamentous fungus. Suitable host cells of the bacterial genera include, but are not limited to, cells of Escherichia, Bacillus, Lactobacillus, Pseudomonas, and Streptomyces. Suitable cells of bacterial species include, but are not limited to, cells of E. coli, B. subtilis, B. licheniformis, L. brevis, P. aeruginosa, and S. lividans.

Suitable host cells of the genera of yeast include, without limitation, cells of Saccharomyces, Schizosaccharomyces, Candida, Hansenula, Pichia, Kluyveromyces, and Phaffia. Suitable cells of yeast species include, without limitation, cells of Saccharomyces cerevisiae, Schizosaccharomyces pombe, Candida albicans, Hansenula polymorpha, Pichia pastoris, P. canadensis, Kluyveromyces marxianus, and Phaffia rhodozyma.

Suitable host cells of filamentous fungi include all filamentous forms of the subdivision Eumycotina. Suitable cells of filamentous fungal genera include, e.g., cells of Acremonium, Aspergillus, Aureobasidium, Bjerkandera, Ceriporiopsis, Chrysoporium, Coprinus, Coriolus, Corynascus, Chaertomium, Cryptococcus, Filobasidium, Fusarium, Gibberella, Humicola, Magnaporthe, Mucor, Myceliophthora, Mucor, Neocallimastix, Neurospora, Paecilomyces, Penicillium, Phanerochaete, Phlebia, Piromyces, Pleurotus, Scytaldium, Schizophyllum, Sporotrichum, Talaromyces, Thermoascus, Thielavia, Tolypocladium, Trametes, and Trichoderma.

Suitable cells of filamentous fungal species include, without limitation, cells of Aspergillus awamori, Aspergillus fumigatus, Aspergillus foetidus, Aspergillus japonicus, Aspergillus nidulans, Aspergillus niger, Aspergillus oryzae, Chrysosporium lucknowense, Fusarium bactridioides, Fusarium cerealis, Fusarium crookwellense, Fusarium culmorum, Fusarium graminearum, Fusarium graminum, Fusarium heterosporum, Fusarium negundi, Fusarium oxysporum, Fusarium reticulatum, Fusarium roseum, Fusarium sambucinum, Fusarium sarcochroum, Fusarium sporotrichioides, Fusarium sulphureum, Fusarium torulosum, Fusarium trichothecioides, Fusarium venenatum, Bjerkandera adusta, Ceriporiopsis aneirina, Ceriporiopsis aneirina, Ceriporiopsis caregiea, Ceriporiopsis gilvescens, Ceriporiopsis pannocinta, Ceriporiopsis rivulosa, Ceriporiopsis subrufa, Ceriporiopsis subvermispora, Coprinus cinereus, Coriolus hirsutus, Humicola insolens, Humicola lanuginosa, Mucor miehei, Myceliophthora thermophila, Neurospora crassa, Neurospora intermedia, Penicillium purpurogenum, Penicillium canescens, Penicillium solitum, Penicillium funiculosum Phanerochaete chrysosporium, Phlebia radiate, Pleurotus eryngii, Talaromyces flavus, Thielavia terrestris, Trametes villosa, Trametes versicolor, Trichoderma harzianum, Trichoderma koningii, Trichoderma longibrachiatum, Trichoderma reesei, or Trichoderma viride.

The disclosure further provides a recombinant host cell engineered to express, in a first aspect, (1) a first polypeptide having xylanase activity, (2) a second polypeptide having xylosidase activity, (3) a third polypeptide having arabinofuranosidase activity, and (4) a fourth polypeptide having β-glucosidase activity. The disclosure also provides, in a second aspect, a recombinant host cell engineered to express (1) a first polypeptide having xylanase activity, (2) a second polypeptide having xylosidase activity, (3) a third polypeptide having arabinofuranosidase activity, and (4) a β-glucosidase-enriched whole cellulase composition. The disclosure also provides, in a third aspect, a recombinant host cell engineered to express (1) a first polypeptide having xylanase activity; (2) a second polypeptide having xylosidase activity; (3) a third polypeptide having arabinofuranosidase activity; and (4) a fourth polypeptide having a GH61/endoglucanase activity, or a GH61 endoglucanase-enriched whole cellulase.

The disclosure provides, in a fourth aspect, a recombinant host cell engineered to express (1) a first polypeptide having xylosidase activity, (2) a second polypeptide (which differs from the first polypeptide) having xylosidase activity, (3) a third polypeptide having arabinofuranosidase activity, and (4) a fourth polypeptide having β-glucosidase activity. The disclosure provides, in a fifth aspect, a recombinant host cell engineered to express (1) a first polypeptide having xylosidase activity, (2) a second polypeptide (different from the first polypeptide) having xylosidase activity, (3) a third polypeptide having arabinofuranosidase activity, and (4) a β-glucosidase enriched whole cellulase. The disclosure further provides, in a sixth aspect, a host cell engineered to express (1) a first polypeptide having xylosidase activity, (2) a second polypeptide (which differs from the first polypeptide) having xylosidase activity, (3) a third polypeptide having arabinofuranosidase activity; (4) a fourth polypeptide having GH61/endoglucanase activity, or alternatively an EGIV-enriched whole cellulase.

The disclosure provides, in a seventh aspect, a recombinant host cell that is engineered to express (1) a first polypeptide having xylanase activity, (2) a second polypeptide having xylosidase activity, (3) a third polypeptide (different from the second polypeptide) having xylosidase activity, and (4) a fourth polypeptide having β-glucosidase activity. The disclosure provides, in an eighth aspect, a recombinant host cell that is engineered to express (1) a first polypeptide having xylanase activity, (2) a second polypeptide having xylosidase activity, (3) a third polypeptide (different from the second polypeptide) having xylosidase activity, and a β-glucosidase enriched whole cellulase. The disclosure provides, in a nineth aspect, a recombinant host cell that is engineered to express (1) a first polypeptide having xylanase activity, (2) a second polypeptide having xylosidase activity, (3) a third polypeptide (different from the second polypeptide) having xylosidase activity, and (4) a fourth polypeptide having GH61/endoglucanase activity, or alternatively a GH61 endoglucanse-enriched whole cellulase.

The disclosure provides, in tenth aspect, a recombinant host cell engineered to express (1) a first polypeptide having xylanase activity, (2) a second polypeptide having xylosidase activity, and (3) a third polypeptide having β-glucosidase activity. The disclosure provides, in an eleventh aspect, a recombinant host cell that is engineered to express (1) a first polypeptide having xylanase activity, (2) a second polypeptide having xylosidase activity, and a β-glucosidase enriched whole cellulase. The disclosure also provides, in a twelveth aspect, a recombinant host cell that is engineered to express (1) a first polypeptide having xylanase activity, (2) a second polypeptide having xylosidase activity, and (3) a third polypeptide having GH61/endoglucanase activity, or alternatively, a GH61 endoglucanase-enriched whole cellulase.

In a recombinant host cell of any of the first to twelveth aspects above, the polypeptide having β-glucosidase activity is one that has at least about 60% (e.g., at least about 60%, 65%, 70%, 75%, 80%, 85%, 90%, 91%, 92%, 93%, 94%, 95%, 96%, 97%, 98%, or 99%) sequence identity to any one of SEQ ID NOs: 54, 56, 58, 60, 62, 64, 66, 68, 70, 72, 74, 76, 78, 79, 93, and 95, over a region of at least about 10 (e.g., at least about 10, 15, 20, 25, 30, 35, 40, 45, 50, 55, 60, 65, 70, 75, 80, 85, 90, 95, 100, 125, 150, 175, 200, 225, 250, 275, 300) residues. In certain embodiments, the polypeptide having β-glucosidase is a chimeric/fusion β-glucosidase polypeptide comprising two or more β-glucosidase sequences, wherein the first sequence derived from a first β-glucosidase is at least about 200 amino acid residues in length and comprises one or more or all of the amino acid sequence motifs of SEQ ID NOs: 96-108, whereas the second sequence derived from a second β-glucosidase is at least about 50 amino acid residues in length and comprises one or more or all of the amino acid sequence motifs of SEQ ID NOs:109-116, and optionally also a third sequence of 3, 4, 5, 6, 7, 8, 9, 10, or 11 amino acid residues in length encoding a loop sequence having an amino acid sequence of FDRRSPG (SEQ ID NO:204), or of FD(R/K)YNIT (SEQ ID NO:205), derived from a third β-glucosidase is a fusion or chimeric β-glucosidase polypeptide. In particular, the first of the two or more β-glucosidase sequences is one that is at least about 200 amino acid residues in length and comprises at least 2 (e.g., at least 2, 3, 4, or all) of the amino acid sequence motifs of SEQ ID NOs: 197-202, and the second of the two or more β-glucosidase is at least 50 amino acid residues in length and comprises SEQ ID NO:203, and optionally also a third sequence of about 3, 4, 5, 6 ,7 ,8 , 9, 10, or 11 amino acid residues in length and having an amino acid sequence of FDRRSPG (SEQ ID NO:204), or of FD(R/K)YNIT (SEQ ID NO:205), which is derived from a third β-glucosidase polypeptide different from the first or the second β-glucosidase polypeptide. In certain embodiments, the polypeptide having β-glucosidase activity is one that comprises a first sequence having least about 60% sequence identity to an at least 200-residue stretch of Fv3C (SEQ ID NO:60), for example, an at least 200-residue stretch from the N-terminus of SEQ ID NO:60, and a second sequence having at least about 60% sequence identity to an at least 50-residue stretch of T. reesei Bgl3 (Tr3B, SEQ ID NO:64), for example, an at least 50-residue stretch from the C-terminus of SEQ ID NO:64. In certain embodiments, the polypeptide having β-glucosidase activity comprising the first and second sequences as above further comprises a third sequence of about 3, 4, 5, 6, 7, 8, 9, 10, or 11 amino acid residues that is derived from a sequence of equal length from Te3A (SEQ ID NO:66), having, e.g., an amino acid sequence of FDRRSPG (SEQ ID NO:204), or of FD(R/K)YNIT (SEQ ID NO:205). In some embodiments, the polypeptide comprises a sequence that has at least about 60% sequence identity to SEQ ID NO:93 or 95, or to a subsequence or fragment of at least about 20, 30, 40, 50, 60, 70, or more residues of SEQ ID NO: 93 or 95.

In a recombinant host cell of any of the first to twelveth aspects above, the recombinant host cell is engineered to express a polypeptide having GH61/endoglucanase activity. In some embodiments, the polypeptide having GH61/endoglucanase activity is an EGIV polypeptide, e.g., a T. reesei Eg4 polypeptide. In some embodiments, the polypeptide is one having at least about 60% (e.g., at least about 60%, 65%, 70%, 75%, 80%, 85%, 90%, 91%, 92%, 93%, 94%, 95%, 96%, 97%, 98%, or 99%) sequence identity to any one of SEQ ID NOs: 52, 80-81, 206-207, over a region of at least about 10 (e.g., at least about 10, 15, 20, 25, 30, 35, 40, 45, 50, 55, 60, 65, 70, 75, 80, 85, 90, 95, 100, 125, 150, 175, 200, 225, 250, 275, 300) residues, or one that comprises one or more sequence motifs selected from the group consisting of: (1) SEQ ID NOs:84 and 88; (2) SEQ ID NOs:85 and 88; (3) SEQ ID NO:86; (4) SEQ ID NO:87; (5) SEQ ID NOs:84, 88 and 89; (6) SEQ ID NOs:85, 88, and 89; (7) SEQ ID NOs: 84, 88, and 90; (8) SEQ ID NOs: 85, 88 and 90; (9) SEQ ID NOs:84, 88 and 91; (10) SEQ ID NOs: 85, 88 and 91; (11) SEQ ID NOs: 84, 88, 89 and 91; (12) SEQ ID NOs: 84, 88, 90 and 91; (13) SEQ ID NOs: 85, 88, 89 and 91: and (14) SEQ ID NOs: 85, 88, 90 and 91. In certain embodiments, the recombinant host cell can be engineered to also express a cellobiose dehydrogenase.

In a recombinant host cell of any of the first to twelth aspects above, the recombinant host cell is engineered to express a polypeptide having xylosidase activity, which is selected from Group 1 β-xylosidase polypeptides. Group 1 β-xylosidase polypeptides includes those having at least about 70% sequence identity to any one of SEQ ID NOs: 2 and 10, or to a mature sequences thereof. For example, Group β-xylosidase may be Fv3A or Fv43A. The recombinant host cell may also be engineered to express a polypeptide having xylosidase activity, which is one selected from Group 2 β-xylosidase polypeptides. Group 2 β-xylosidase polypeptides include those having at least about 70% sequence identity to any one of SEQ ID NOs:4, 6, 8, 10, 12, 14, 16, 18, 28, 30, and 45, or to a mature sequence thereof. For example, Group 2 β-xylosidases may be Pf43A, Fv43E, Fv39A, Fv43B, Pa51A, Gz43A, Fo43A, Fv43D, Pf43B, or T. reesei Bxl1.

In a recombinant host cells of any the first, second, and third aspects above, the polypeptide having xylanase activity is one having at least about 70% sequence identity to any one of SEQ ID NOs: 24, 26, 42, and 43, or to a mature sequence thereof. For example, the xylanase polypeptide can be AfuXyn2, AfuXyn5, T. reesei Xyn3 or T. reesei Xyn2.

In a recombinant host cell of any of the fourth, fifth and sixth aspects, the host cell may be engineered to express a polypeptide having arabinofuranosidase activity, which has at least about 70% sequence identity to any one of SEQ ID NOs:12, 14, 20, 22, and 32, or to a mature sequence thereof. For example, the third polypeptide can be Fv43B, Pa51A, Af43A, Pf51A, or Fv51A.

The recombinant host cell of the disclosure can suitably be, e.g., a recombinant fungal host cell or a recombinant organism, e.g., a filamentous fungus, such as a recombinant T. reesei. For example, the recombinant host cell is suitably a Trichoderma reesei host cell. The recombinant fungus is suitably a recombinant Trichoderma reesei. The disclosure provides, e.g., a T. reesei host cell.

Additionally the disclosure provides a recombinant host cell or recombinant fungus that is engineered to express an enzyme blend comprising suitable enzymes in ratios suitable for saccharification. The recombinant host cell is, e.g., a fungal host cell. The recombinant fungus is, e.g., a recombinant Trichoderma reesei, Aspergillus niger or Aspergillus oryzae, or Chrisosporium lucknowence. The recombinant bacterial host cell may be a Bacillus cell. Examples of suitable enzyme ratios/amounts present in the enzyme blends are described in Section 5.3.4.

5.3 Enzyme Compositions for Saccharification

The present disclosure provides an enzyme composition that is capable of breaking down lignocellulose material. The enzyme composition of the invention is typically a multi-enzyme blend, comprising more than one enzymes or polypeptides of the disclosure. The enzyme composition of the invention can suitably include one or more additional enzymes derived from other microorganisms, plants, or organisms. Synergistic enzyme combinations and related methods are contemplated. The disclosure includes methods for identifying the optimum ratios of the enzymes included in the enzyme compositions for degrading various types of lignocellulosic materials. These methods include, e.g., tests to identify the optimum proportion or relative weights of enzymes to be included in the enzyme composition of the invention in order to effectuate efficient conversion of various lignocellulosic substrates to their constituent fermentable sugars. The Examples below include assays that may be used to identify optimum proportions/relative weights of enzymes in the enzyme compositions, with which to various lignocellulosic materials are efficienty hydrolyzed or broken down in saccharification processes.

5.3.1. Background

The cell walls of higher plants comprise a variety of carbohydrate polymer (CP) components. These CP interact through covalent and non-covalent means, providing the structural integrity required to form rigid cell walls and resist turgor pressure in plants. The major CP found in plants is cellulose, which forms the structural backbone of the cell wall. During cellulose biosynthesis, chains of poly-β-1,4-D-glucose self associate through hydrogen bonding and hydrophobic interactions to form cellulose microfibrils, which further self-associate to form larger fibrils. Cellulose microfibrils are often irregular structurally and contain regions of varying crystallinity. The degree of crystallinity of cellulose fibrils depends on how tightly ordered the hydrogen bonding is between and among its component cellulose chains. Areas with less-ordered bonding, and therefore more accessible glucose chains, are referred to as amorphous regions.

The general model for cellulose depolymerization to glucose involves a minimum of three distinct enzymatic activities. Endoglucanases cleave cellulose chains internally to shorter chains in a process that increases the number of accessible ends, which are more susceptible to exoglucanase activity than the intact cellulose chains. These exoglucanases (e.g., cellobiohydrolases) are specific for either reducing ends or non-reducing ends, liberating, in most cases, cellobiose, the dimer of glucose. The accumulating cellobiose is then subject to cleavage by cellobiases (e.g., β-1,4-glucosidases) to glucose.

Cellulose contains only anhydro-glucose. In contrast, hemicellulose contains a number of different sugar monomers. For instance, aside from glucose, sugar monomers in hemicellulose can also include xylose, mannose, galactose, rhamnose, and arabinose. Hemicelluloses mostly contain D-pentose sugars and occasionally small amounts of L-sugars. Xylose is typically present in the largest amount, but mannuronic acid and galacturonic acid also tend to be present. Hemicelluloses include xylan, glucuronoxylan, arabinoxylan, glucomannan, and xyloglucan.

The enzymes and multi-enzyme compositions of the disclosure are useful for saccharification of hemicellulose materials, including, e.g., xylan, arabinoxylan, and xylan- or arabinoxylan-containing substrates. Arabinoxylan is a polysaccharide composed of xylose and arabinose, wherein L-α-arabinofuranose residues are attached as branch-points to a β-(1,4)-linked xylose polymeric backbone.

Most biomass sources are rather complex, containing cellulose, hemicellulose, pectin, lignin, protein, and ash, among other components. Accordingly, in certain aspects, the present disclosure provides enzyme blends/compositions containing enzymes that impart a range or variety of substrate specificities when working together to degrade biomass into fermentable sugars in the most efficient manner. One example of a multi-enzyme blend/composition of the present invention is a mixture of cellobiohydrolase(s), xylanase(s), endoglucanase(s), β-glucosidase(s), β-xylosidase(s), and, optionally, accessory proteins. The enzyme blend/composition is suitably a non-naturally occurring composition.

Accordingly, the disclosure provides enzyme blends/compositions (including products of manufacture) comprising a mixture of xylan-hydrolyzing, hemicellulose- and/or cellulose-hydrolyzing enzymes, which include at least one, several, or all of a cellulase, including a glucanase; a cellobiohydrolase; an L-α-arabinofuranosidase; a xylanase; a β-glucosidase; and a β-xylosidase. Preferably each of the enzyme blends/compositions of the disclosure comprises at least one enzyme of the disclosure. The present disclosure also provides enzyme blends/compositions that are non-naturally occurring compositions. As used herein, the term “enzyme blends/compositions” refers to: (1) a composition made by combining component enzymes, whether in the form of a fermentation broth or partially or completely isolated or purified; (2) a composition produced by an organism modified to express one or more component enzymes; in certain embodiments, the organism used to express one or more component enzymes can be modified to delete one or more genes; in certain other embodiments, the organism used to express one or more component enzymes can further comprise proteins affecting xylan hydrolysis, hemicellulose hydrolysis, and/or cellulose hydrolysis; (3) a composition made by combining component enzymes simultaneously, separately, or sequentially during a saccharification or fermentation reaction; (4)an enzyme mixture produced in situ, e.g., during a saccharification or fermentation reaction; and (5) a composition produced in accordance with any or all of the above (1)-(4).

The term “fermentation broth” as used herein refers to an enzyme preparation produced by fermentation that undergoes no or minimal recovery and/or purification subsequent to fermentation. For example, microbial cultures are grown to saturation, incubated under carbon-limiting conditions to allow protein synthesis (e.g., expression of enzymes). Then, once the enzyme(s) are secreted into the cell culture media, the fermentation broths can be used. The fermentation broths of the disclosure can contain unfractionated or fractionated contents of the fermentation materials derived at the end of the fermentation. For example, the fermentation broths of the invention are unfractionated and comprise the spent culture medium and cell debris present after the microbial cells (e.g., filamentous fungal cells) undergo a fermentation process. The fermentation broth can suitably contain the spent cell culture media, extracellular enzymes, and live or killed microbial cells. Alternatively, the fermentation broths can be fractionated to remove the microbial cells. In those cases, the fermentation broths can, for example, comprise the spent cell culture media and the extracellular enzymes.

Any of the enzymes described specifically herein can be combined with any one or more of the enzymes described herein or with any other available and suitable enzymes, to produce a suitable multi-enzyme blend/composition. The disclosure is not restricted or limited to the specific exemplary combinations listed below.

5.3.2. Biomass

The disclosure provides methods and processes for biomass saccharification, using enzymes, enzyme blends/compositions of the disclosure. The term “biomass,” as used herein, refers to any composition comprising cellulose and/or hemicellulose (optionally also lignin in lignocellulosic biomass materials). As used herein, biomass includes, without limitation, seeds, grains, tubers, plant waste or byproducts of food processing or industrial processing (e.g., stalks), corn (including, e.g., cobs, stover, and the like), grasses (including, e.g., Indian grass, such as Sorghastrum nutans; or, switchgrass, e.g., Panicum species, such as Panicum virgatum), perennial canes (e.g., giant reeds), wood (including, e.g., wood chips, processing waste), paper, pulp, and recycled paper (including, e.g., newspaper, printer paper, and the like). Other biomass materials include, without limitation, potatoes, soybean (e.g., rapeseed), barley, rye, oats, wheat, beets, and sugar cane bagasse.

The disclosure provides methods of saccharification comprising contacting a composition comprising a biomass material, e.g., a material comprising xylan, hemicellulose, cellulose, and/or a fermentable sugar, with a polypeptide of the disclosure, or a polypeptide encoded by a nucleic acid of the disclosure, or any one of the enzyme blends/compositions, or products of manufacture of the disclosure.

The saccharified biomass (e.g., lignocellulosic material processed by enzymes of the disclosure) can be made into a number of bio-based products, via processes such as, e.g., microbial fermentation and/or chemical synthesis. As used herein, “microbial fermentation” refers to a process of growing and harvesting fermenting microorganisms under suitable conditions. The fermenting microorganism can be any microorganism suitable for use in a desired fermentation process for the production of bio-based products. Suitable fermenting microorganisms include, without limitation, fungi (e.g., filamentous fungi), yeast, and bacteria. The saccharified biomass can, e.g., be made it into a fuel (e.g., a biofuel such as a bioethanol, biobutanol, biomethanol, a biopropanol, a biodiesel, a jet fuel, or the like) via fermentation and/or chemical synthesis. The saccharified biomass can, e.g., also be made into a commodity chemical (e.g., ascorbic acid, isoprene, 1,3-propanediol), lipids, amino acids, proteins, and enzymes, via fermentation and/or chemical synthesis.

5.3.3. Pretreatment

Prior to saccharification, biomass (e.g., lignocellulosic material) is preferably subject to one or more pretreatment step(s) in order to render xylan, hemicellulose, cellulose and/or lignin material more accessible or susceptable to enzymes and thus more amenable to hydrolysis by the enzyme(s) and/or enzyme blends/compositions of the disclosure.

In certain embodiments, the pretreatment entails subjecting the biomass material to a catalyst comprising a dilute solution of a strong acid and a metal salt in a reactor. The biomass material can, e.g., be a raw material or a dried material. This pretreatment can lower the activation energy, or the temperature, of cellulose hydrolysis, ultimately allowing higher yields of fermentable sugars. See, e.g., U.S. Pat. Nos. 6,660,506; 6,423,145.

Another example of a pretreatment involves hydrolyzing biomass by subjecting the biomass material to a first hydrolysis step in an aqueous medium at a temperature and a pressure chosen to effectuate primarily depolymerization of hemicellulose without achieving significant depolymerization of cellulose into glucose. This step yields a slurry in which the liquid aqueous phase contains dissolved monosaccharides resulting from depolymerization of hemicellulose, and a solid phase containing cellulose and lignin. The slurry is then subject to a second hydrolysis step under conditions that allow a major portion of the cellulose to be depolymerized, yielding a liquid aqueous phase containing dissolved/soluble depolymerization products of cellulose. See, e.g., U.S. Pat. No. 5,536,325.

A further example of a method involves processing a biomass material by one or more stages of dilute acid hydrolysis using about 0.4% to about 2% of a strong acid; followed by treating the unreacted solid lignocellulosic component of the acid hydrolyzed material with alkaline delignification. See, e.g., U.S. Pat. No. 6,409,841.

Another example of a method comprises prehydrolyzing biomass (e.g., lignocellulosic materials) in a prehydrolysis reactor; adding an acidic liquid to the solid lignocellulosic material to make a mixture; heating the mixture to reaction temperature; maintaining reaction temperature for a period of time sufficient to fractionate the lignocellulosic material into a solubilized portion containing at least about 20% of the lignin from the lignocellulosic material, and a solid fraction containing cellulose; separating the solubilized portion from the solid fraction, and removing the solubilized portion while at or near the reaction temperature; and recovering the solubilized portion. The cellulose in the solid fraction is rendered more amenable to enzymatic digestion. See, e.g., U.S. Pat. No. 5,705,369.

Further pretreatment methods can involve the use of hydrogen peroxide H₂O₂. See Gould, 1984, Biotech, and Bioengr. 26:46-52.

Pretreatment can also comprise contacting a biomass material with stoichiometric amounts of sodium hydroxide and ammonium hydroxide at a very low concentration. See Teixeira et al., 1999, Appl. Biochem. and Biotech. 77-79:19-34. Pretreatment can also comprise contacting a lignocellulose with a chemical (e.g., a base, such as sodium carbonate or potassium hydroxide) at a pH of about 9 to about 14 at moderate temperature, pressure, and pH. See PCT Publication WO2004/081185.

Ammonia is used, e.g., in a preferred pretreatment method. Such a pretreatment method comprises subjecting a biomass material to low ammonia concentration under conditions of high solids. See, e.g., U.S. Patent Publication No. 20070031918 and PCT publication WO 06110901.

5.3.4. Enzyme Compositions

The present disclosure provides a number of enzyme compositions comprising multiple (i.e., more than one) enzymes of the disclosure. At least one enzyme of each of the enzyme composition of the invention can be produced by a recombinant host cell or a recombinant organism. At least one enzyme of the enzyme composition can be an exogenous enzyme, produced by, e.g., expressing an exogenous gene in a host cell or a host organism. At least one enzyme of the enzyme composition can be produced as a result of overexpressing or underexpressing an endogenous gene in a host cell or host organism. The enzyme compositions are suitably non-naturally occurring compositions. The disclosure provides a first non-limiting example of an engineered enzyme composition of the invention comprising 4 polypeptides: (1) a first polypeptide having xylanase activity, (2) a second polypeptide having xylosidase activity, (3) a third polypeptide having arabinofuranosidase activity, and (4) a fourth polypeptide having β-glucosidase activity. The disclosure provides a second non-limiting example of an engineered enzyme composition of the invention comprising:(1) a first polypeptide having xylanase activity, (2) a second polypeptide having xylosidase activity, (3) a third polypeptide having arabinofuranosidase activity, and (4) a β-glucosidase-enriched whole cellulase composition. The disclosure provides a third non-limiting example of an engineered enzyme composition of the invention comprising (1) a first polypeptide having xylanase activity; (2) a second polypeptide having xylosidase activity; (3) a third polypeptide having arabinofuranosidase activity; and (4) a fourth polypeptide having a GH61/endoglucanase activity, or a GH61 endoglucanase-enriched whole cellulase. The disclosure provides a fourth non-limiting example of an engineered enzyme composition of the invention comprising (1) a first polypeptide having xylosidase activity, (2) a second polypeptide (which differs from the first polypeptide) having xylosidase activity, (3) a third polypeptide having arabinofuranosidase activity, and (4) a fourth polypeptide having β-glucosidase activity. The disclosure provides a fifth non-limiting example of an enzyme composition of the invention comprising (1) a first polypeptide having xylosidase activity, (2) a second polypeptide (different from the first polypeptide) having xylosidase activity, (3) a third polypeptide having arabinofuranosidase activity, and (4) a β-glucosidase enriched whole cellulase. The disclosure provides a sixth non-limiting example of an engineered enzyme composition of the invention comprising (1) a first polypeptide having xylosidase activity, (2) a second polypeptide (which differs from the first polypeptide) having xylosidase activity, (3) a third polypeptide having arabinofuranosidase activity; and (4) a fourth polypeptide having GH61/endoglucanase activity, or alternatively, an EGIV-enriched whole cellulase. The disclosure provides a seventh non-limiting example of an engineered enzyme composition of the invention comprising(1) a first polypeptide having xylanase activity, (2) a second polypeptide having xylosidase activity, (3) a third polypeptide (different from the second polypeptide) having xylosidase activity, and (4) a fourth polypeptide having β-glucosidase activity. The disclosure provides an eighth non-limiting example comprising (1) a first polypeptide having xylanase activity, (2) a second polypeptide having xylosidase activity, (3) a third polypeptide (different from the second polypeptide) having xylosidase activity, and a β-glucosidase enriched whole cellulase. The disclosure provides a ninth non-limiting example of an engineered enzyme composition of the invention comprising (1) a first polypeptide having xylanase activity, (2) a second polypeptide having xylosidase activity, (3) a third polypeptide (different from the second polypeptide) having xylosidase activity, and (4) a fourth polypeptide having GH61/endoglucanase activity, or alternatively a GH61 endoglucanse-enriched whole cellulase. The disclosure provides a tenth non-limiting example of an engineered enzyme composition of the invention comprising (1) a first polypeptide having xylanase activity, (2) a second polypeptide having xylosidase activity, and (3) a third polypeptide having β-glucosidase activity. The disclosure provides an eleventh non-limiting example of an enzyme composition of the invention comprising (1) a first polypepti8e having xylanase activity, (2) a second polypeptide having xylosidase activity, and a β-glucosidase enriched whole cellulase. The disclosure provides a twelveth non-limiting example of an engineered enzyme composition of the invention comprising (1) a first polypeptide having xylanase activity, (2) a second polypeptide having xylosidase activity, and (3) a third polypeptide having GH61/endoglucanase activity, or alternatively, a GH61 endoglucanase-enriched whole cellulase.

In any one of the exemplary enzyme compositions above, the polypeptide having β-glucosidase activity is one that has at least about 60% (e.g., at least about 60%, 65%, 70%, 75%, 80%, 85%, 90%, 91%, 92%, 93%, 94%, 95%, 96%, 97%, 98%, or 99%) sequence identity to any one of SEQ ID NOs: 54, 56, 58, 60, 62, 64, 66, 68, 70, 72, 74, 76, 78, 79, 93, and 95, over a region of at least about 10 (e.g., at least about 10, 15, 20, 25, 30, 35, 40, 45, 50, 55, 60, 65, 70, 75, 80, 85, 90, 95, 100, 125, 150, 175, 200, 225, 250, 275, 300) residues.

In certain embodiments, the polypeptide having β-glucosidase is a chimeric/fusion β-glucosidase polypeptide comprising two or more β-glucosidase sequences, wherein the first sequence derived from a first β-glucosidase is at least about 200 amino acid residues in length and comprises one or more or all of the amino acid sequence motifs of SEQ ID NOs: 96-108, whereas the second sequence derived from a second β-glucosidase is at least about 50 amino acid residues in length and comprises one or more or all of the amino acid sequence motifs of SEQ ID NOs:109-116, and optionally also a third sequence of 3, 4, 5, 6, 7, 8, 9, 10, or 11 amino acid residues in length encoding a loop sequence derived from a third β-glucosidase is a fusion or chimeric β-glucosidase polypeptide. In certain embodiments, the polypeptide having β-glucosidase activity is one that comprises a first sequence having least about 60% sequence identity to an at least 200-residue stretch of Fv3C (SEQ ID NO:60), for example, an at least 200-residue stretch from the N-terminus of SEQ ID NO:60, and a second sequence having at least about 60% sequence identity to an at least 50-residue stretch of T. reesei Bgl3 (Tr3B, SEQ ID NO:64), for example, an at least 50-residue stretch from the C-terminus of SEQ ID NO:64. In certain embodiments, the polypeptide having β-glucosidase activity comprising the first and second sequences as above further comprises a third sequence of about 3, 4, 5, 6, 7, 8, 9, 10, or 11 amino acid residues that is derived from a sequence of equal length from Te3A (SEQ ID NO:66). In some embodiments, the polypeptide comprises a sequence that has at least about 60% sequence identity to SEQ ID NO:93 or 95, or to a subsequence or fragment of at least about 20, 30, 40, 50, 60, 70, or more residues of SEQ ID NO: 93 or 95.

In any one of the enzyme compositions herein, the polypeptide having GH61/endoglucanase activity is an EGIV polypeptide, e.g., a T. reesei Eg4 polypeptide. In some embodiments, the polypeptide is one having at least about 60% (e.g., at least about 60%, 65%, 70%, 75%, 80%, 85%, 90%, 91%, 92%, 93%, 94%, 95%, 96%, 97%, 98%, or 99%) sequence identity to any one of SEQ ID NOs: 52, 80-81, 206-207, over a region of at least about 10 (e.g., at least about 10, 15, 20, 25, 30, 35, 40, 45, 50, 55, 60, 65, 70, 75, 80, 85, 90, 95, 100, 125, 150, 175, 200, 225, 250, 275, 300) residues, or one that comprises one or more sequence motifs selected from the group consisting of: (1) SEQ ID NOs:84 and 88; (2) SEQ ID NOs:85 and 88; (3) SEQ ID NO:86; (4) SEQ ID NO:87; (5) SEQ ID NOs:84, 88 and 89; (6) SEQ ID NOs:85, 88, and 89; (7) SEQ ID NOs: 84, 88, and 90; (8) SEQ ID NOs: 85, 88 and 90; (9) SEQ ID NOs:84, 88 and 91; (10) SEQ ID NOs: 85, 88 and 91; (11) SEQ ID NOs: 84, 88, 89 and 91; (12) SEQ ID NOs: 84, 88, 90 and 91; (13) SEQ ID NOs: 85, 88, 89 and 91: and (14) SEQ ID NOs: 85, 88, 90 and 91. In certain embodiments, the composition further comprises a cellobiose dehydrogenase.

In any one of the enzyme compositions herein, the polypeptide having xylanase activity may be one that has at least about 70% sequence identity to any one of SEQ ID NOs: 24, 26, 42, and 43, or to a mature sequence thereof. For example, the xylanase polypeptide can be AfuXyn2, AfuXyn5, T. reesei Xyn3, or T. reesei Xyn2.

In any one of the enzyme compositions herein, the polypeptide having xylosidase activity can be one selected from a Group 1 or Group 2 β-xylosidase polypeptides. When the composition comprises a first and a second β-xylosidases, it is contemplated that the first β-xylosidase is a Group 1 β-xylosidase polypeptide, which can be one that has at least about 70% sequence identity to any one of SEQ ID NOs: 2 and 10, or to mature sequences thereof. For example, Group 1 β-xylosidase can be Fv3A, or Fv43A. It is also contemplated that the second β-xylosidase is a Group 2 β-xylosidase polypeptide, which can be one having at least about 70% sequence identity to any one of SEQ ID NOs:4, 6, 8, 10, 12, 14, 16, 18, 28, 30, and 45, or to a mature sequence thereof. For example, Group 2 β-xylosidases can be Pf43A, Fv43E, Fv39A, Fv43B, Pa51A, Gz43A, Fo43A , Fv43D, Pf43B, or T. reesei Bxl1.

In any one of the examples of the enzyme compositions above, the polypeptide having arabinofuranosidase activity can be one that has at least about 70% sequence identity to any one of SEQ ID NOs:12, 14, 20, 22, and 32, or to a mature sequence thereof. For example, the third polypeptide can be Fv43B, Pa51A, Af43A, Pf51A, or Fv51A.

Xylanases: The xylanase(s) suitably constitutes about 3 wt. % to about 35 wt. % of the enzymes in an enzyme composition of the disclosure, wherein the wt. % represents the combined weight of xylanase(s) relative to the combined weight of all enzymes in a given composition. The xylanase(s) can be present in a range wherein the lower limit is 3 wt. %, 4 wt. %, 5 wt. %, 6 wt. %, 7 wt. %, 8 wt. %, 9 wt. %, 10 wt. %, 12 wt. %, 15 wt. %, and the upper limit is 5 wt. %, 10 wt. %,15 wt. %, 20 wt. %, 25 wt. %, 30 wt. %, 35 wt. %. Suitably, the combined weight of one or more xylanases in an enzyme composition of the invention can constitute, e.g., about 3 wt. % to about 30 wt. % (e.g., 3 wt. % to 20 wt. %, 5 wt. % to 18 wt. %, 8 wt. % to 18 wt. %, 10 wt. % to 20 wt. % etc) of the total weight of all enzymes in the enzyme composition. Examples of suitable xylanases for inclusion in the enzyme compositions of the disclosure are described in Section 5.3.7.

L-α-arabinofuranosidases: The L-α-arabinofuranosidase(s) suitably constitutes about 0.1 wt. % to about 5 wt. % of the enzymes in an enzyme composition of the disclosure, wherein the wt. % represents the combined weight of L-α-arabinofuranosidase(s) relative to the combined weight of all enzymes in a given composition. The L-α-arabinofuranosidase(s) can be present in a range wherein the lower limit is 0.1 wt. %, 0.2 wt. %, 0.5 wt. %, 0.7 wt. %, 0.8 wt. %, 1 wt. %, 2 wt. %, 3 wt. %, 4 wt, and the upper limit is 2 wt. %, 3 wt. %, 4 wt. %, or 5 wt. For example, the one or more L-α-arabinofuranosidase(s) can suitably constitute about 0.2 wt. % to about 5 wt. % (e.g., 0.2 wt. % to 3 wt. %, 0.4 wt. % to 2 wt. %, 0.4 wt. % to 1 wt. % etc) of the total weight of enzymes in an enzyme composition of the invention. Examples of suitable L-α-arabinofuranosidase(s) for inclusion in the enzyme blends compositions of the disclosure are described in Section 5.3.8.

β-Xylosidases: The β-xylosidase(s) suitably constitutes about 0 wt. % to about 40 wt. % of the total weight of enzymes in an enzyme blend/composition. The amount can be calculated using known methods, such as, e.g., SDS-PAGE, H PLC, and UPLC, as in the Examples. The ratio of any pair of proteins relative to each other can be readily calculated. Blends/compositions comprising enzymes in any weight ratio derivable from the weight percentages disclosed herein are contemplated. The β-xylosidase content can be in a range wherein the lower limit is about 0 wt. %, 1 wt. %, 2 wt. %, 3 wt. %, 4 wt. %, 5 wt. %, 6 wt. % 7 wt. %, 8 wt. %, 9 wt. %, 10 wt. %, 12 wt. %, 15 wt. %, 20 wt. %, 25 wt. %, 30 wt. %, 35 wt. % of the total weight of enzymes in the blend/composition, and the upper limit is about 10 wt,%, 15 wt,%, 20 wt. %, 25 wt. %, 30 wt. %, 35 wt. %, or 40 wt. % of the total weight of enzymes in the blend/composition. For example, the β-xylosidase(s) suitably represent 2 wt. % to 30 wt. %; 10 wt. % to 20 wt. %; or 5 wt. % to 10 wt. % of the total weight of enzymes in the blend/composition. Suitable β-xylosidase(s) are described herein, e.g., in Section 5.3.7.

5.3.5. Cellulases

The enzyme blends/compositions of the disclosure can comprise one or more cellulases. Cellulases are enzymes that hydrolyze cellulose (β-1,4-glucan or β D-glucosidic linkages) resulting in the formation of glucose, cellobiose, cellooligosaccharides, and the like. Cellulases have been traditionally divided into three major classes: endoglucanases (EC 3.2.1.4) (“EG”), exoglucanases or cellobiohydrolases (EC 3.2.1.91) (“CBH”) and β-glucosidases (β-D-glucoside glucohydrolase; EC 3.2.1.21) (“BG”) (Knowles et al., 1987, Trends in Biotechnology 5(9):255-261; Shulein, 1988, Methods in Enzymology, 160:234-242). Endoglucanases act mainly on the amorphous parts of the cellulose fiber, whereas cellobiohydrolases are also able to degrade crystalline cellulose.

Cellulases suitable for the methods and compositions of the disclosure can be obtained from, or produced recombinantly from, inter alia, one or more of the following organisms: Crinipellis scapella, Macrophomina phaseolina, Myceliophthora thermophila, Sordaria fimicola, Volutella colletotrichoides, Thielavia terrestris, Acremonium sp., Exidia glandulosa, Fomes fomentarius, Spongipellis sp., Rhizophlyctis rosea, Rhizomucor pusillus, Phycomyces niteus, Chaetostylum fresenii, Diplodia gossypina, Ulospora bilgramii, Saccobolus dilutellus, Penicillium verruculosum, Penicillium chrysogenum, Thermomyces verrucosus, Diaporthe syngenesia, Colletotrichum lagenarium, Nigrospora sp., Xylaria hypoxylon, Nectria pinea, Sordaria macrospora, Thielavia thermophila, Chaetomium mororum, Chaetomium virscens, Chaetomium brasiliensis, Chaetomium cunicolorum, Syspastospora boninensis, Cladorrhinum foecundissimum, Scytalidium thermophila, Gliocladium catenulatum, Fusarium oxysporum ssp. lycopersici, Fusarium oxysporum ssp. passiflora, Fusarium solani, Fusarium anguioides, Fusarium poae, Humicola nigrescens, Humicola grisea, Panaeolus retirugis, Trametes sanguinea, Schizophyllum commune, Trichothecium roseum, Microsphaeropsis sp., Acsobolus stictoideus spej., Poronia punctata, Nodulisporum sp., Trichoderma sp. (e.g., T. reesei) and Cylindrocarpon sp.

For example, a cellulase for use in the method and/or composition of the disclosure is a whole cellulase and/or is capable of achieving at least 0.1 (e.g. 0.1 to 0.4) fraction product as determined by the calcofluor assay described in Section 6.1.11. below. 5.3.5.1. β-Glucosidases

The enzyme blends/compositions of the disclosure can optionally comprise one or more β-glucosidases. The term “β-glucosidase” as used herein refers to a β-D-glucoside glucohydrolase classified as EC 3.2.1.21, and/or members of certain GH families, including, without limitation, members of GH families 1, 3, 9 or 48, which catalyze the hydrolysis of cellobiose to release β-D-glucose.

Suitable β-glucosidase can be obtained from a number of microorganisms, by recombinant means, or be purchased from commercial sources. Examples of β-glucosidases from microorganisms include, without limitation, ones from bacteria and fungi. For example, a β-glucosidase of the present disclosure may be from a filamentous fungus.

The β-glucosidases can be obtained, or produced recombinantly, from, inter alia, A. aculeatus (Kawaguchi et al. Gene 1996, 173: 287-288), A. kawachi (lwashita et al. Appl. Environ. Microbiol. 1999, 65: 5546-5553), A. oryzae (WO 2002/095014), C. biazotea (Wong et al. Gene, 1998, 207:79-86), P. funiculosum (WO 2004/078919), S. fibuligera (Machida et al. Appl. Environ. Microbiol. 1988, 54: 3147-3155), S. pombe (Wood et al. Nature 2002, 415: 871-880), or T. reesei (e.g., β-glucosidase 1 (U.S. Pat. No. 6,022,725), β-glucosidase 3 (U.S. Pat. No.6,982,159), β-glucosidase 4 (U.S. Pat. No. 7,045,332), β-glucosidase 5 (U.S. Pat. No. 7,005,289), β-glucosidase 6 (U.S. Publication No. 20060258554), β-glucosidase 7 (U.S. Publication No. 20060258554).

The β-glucosidase can be produced by expressing an endogenous or exogenous gene encoding a β-glucosidase. For example, β-glucosidase can be secreted into the extracellular space e.g., by Gram-positive organisms (e.g., Bacillus or Actinomycetes), or eukaryotic hosts (e.g., Trichoderma, Aspergillus, Saccharomyces, or Pichia). The β-glucosidase can be, in some circumstances, overexpressed or underexpressed.

The β-glucosidase can also be obtained from commercial sources. Examples of commercial β-glucosidase preparation suitable for use in the present disclosure include, for example, T. reesei β-glucosidase in Accellerase® BG (Danisco US Inc., Genencor); NOVOZYM™ 188 (a β-glucosidase from A. niger); Agrobacterium sp. β-glucosidase, and T. maritima β-glucosidase from Megazyme (Megazyme International Ireland Ltd., Ireland.).

Moreover, the β-glucosidase can be a component of a whole cellulase, as described in Section 5.3.6.below.

The disclosure provides certain β-glucosidase polypeptides, which are fusion/chimeric polypeptides comprising two or more β-glucosidase sequences. For example, the first β-glucosidase sequence can comprise a sequence of at least about 200 amino acid residues in length, and comprises one or more or all of the sequence motifs: SEQ ID NOs: 96-108.

The second β-glucosidase sequence can comprises a sequence of at least about 50 amino acid residues in length, and comprises one or more or all of the sequence motifs SEQ ID NOs: 109-116. In certain embodiments, the first β-glucosidase sequence is located at the N-terminal of the fusion/chimeric polypeptide whereas the second β-glucosidase seuqnce is located at the C-terminal of the fusion/chimeric polypeptide. In certain embodiments, the first and the second β-glucosidase sequences are immediately adjacent. For example, the C-terminus of the first β-glucosidase sequence is connected to the N-terminus of the second β-glucosidase sequence. In other embodiments, the first and the second β-glucosidase sequences are not immediately adjacent, but rather the first and the second β-glucosidase sequences are connected via a linker domain. In some embodiments, the first β-glucosidase sequence, the second β-glucosidase sequence, or the linker domain can comprise a sequence of about 3, 4, 5,6 ,7, 8, 9, 10, or 11 amino acid residues in length. In certain embodiments, the first β-glucosidase sequence is at least about 200 amino acid residues in length and has at least about 60% sequence identity to an Fv3C sequence of the same length at the N-terminal. In certain embodiments, the second β-glucosidase sequence is at least about 50 amino acid residues in length, and has at least about 60% sequence identity to a sequence of equal length at the C-terminal of any one of SEQ ID NOs:54, 56, 62, 64, 66, 68, 70, 72, 74, 76, 78, and 79. In certain embodiments, the fusion/chimeric β-glucosidase polypeptide has improved stability, e.g., improved proteolytic stability as compared to any oen of the enzymes from which the chimeric parts of the chimeric/fusion polypeptide has been derived. In certain embodiments, the second β-glucosidase sequence is one that is at least about 50 amino acid residues in length, and has at least about 60% sequence identity to a sequence of equal length at the C-terminal of Tr3B. In certain embodiments, the loop sequence, which is in the first β-glucosidase sequence, in the second β-glucosidase sequence, or in the linker motif, is one of 3, 4, 5, 6, 7, 8 ,9, 10, or 11 amino acid residues in length derived from Te3A.

β-glucosidase activity can be determined by a number of suitable means known in the art, such as the assay described by Chen et al., in Biochimica et Biophysica Acta 1992, 121:54-60, wherein 1 pNPG denotes 1 μmoL of Nitrophenol liberated from 4-nitrophenyl-β-D-glucopyranoside in 10 min at 50° C. (122° F.) and pH 4.8.

β-glucosidase(s) suitably constitutes about 0 wt. % to about 55 wt. % of the total weight of enzymes in an enzyme blend/composition of the invention. The amount can be determined using known methods, including, e.g., the SDS-PAGE, HPLC, or UPLC methods in the Examples. The ratio of any pair of proteins relative to each other can be calculated. Blends /compositions comprising enzymes in any weight ratio derivable from the weight percentages disclosed herein are contemplated. The β-glucosidases content can be in a range wherein the lower limit is about 0 wt. %, 1 wt. %, 2 wt. %, 3 wt. %, 4 wt. %, 5 wt. %, 6 wt. % 7 wt. %, 8 wt. %, 9 wt. %, 10 wt. %, 12 wt. %, 15 wt. %, 20 wt. %, 25 wt. %, 30 wt. %, 40 wt. %, 45 wt. %, or 50 wt. % of the total weight of enzymes in the blend/composition, and the upper limit is about 10 wt,%, 15 wt,%, 20 wt. %, 25 wt. %, 30 wt. %, 35 wt. %, 40 wt. %, 50 wt. %, 55 wt. %, of the total weight of enzymes in the blend/composition. For example, the β-glucosidase(s) suitably represent 2 wt. % to 30 wt. %; 10 wt. % to 20 wt. %; or 5 wt. % to 10 wt. % of the total weight of enzymes in the blend/composition.

5.3.5.2. Endoglucanases

The enzyme blends/compositions of the disclosure optionally comprise one or more endoglucanase in addition to the GH61 endoglucanase IV (EGIV) polypeptides described herein. Any endoglucanase (EC 3.2.1.4) can be used, in addition to the EGIV polypeptides in the methods and compositions of the present disclosure. Such an endoglucanse can be produced by expressing an endogenous or exogenous endoglucanase gene. The endoglucanase can be, in some circumstances, overexpressed or underexpressed.

For example, T. reesei EG1 (Penttila et al., Gene 1986, 63:103-112) and/or EG2 (Saloheimo et al., Gene 1988, 63:11-21) are suitably used in the methods and compositions of the present disclosure. A thermostable T. terrestris endoglucanase (Kvesitadaze et al., Applied Biochem. Biotech. 1995, 50:137-143) is, e.g., used in the methods and compositions of the present disclosure. Moreover, a T. reesei EG3 (Okada et al. Appl. Environ. Microbiol. 1988, 64:555-563), EG5 (Saloheimo et al. Molecular Microbiology 1994, 13:219-228), EG6 (U.S. Patent Publication No. 20070213249), or EG7 (U.S. Patent Publication No. 20090170181), an A. cellulolyticus El endoglucanase (U.S. Pat. No. 5,536,655), a H. insolens endoglucanase V (EGV) (Protein Data Bank entry 4ENG), a S. coccosporum endoglucanase (U.S. Patent Publication No. 20070111278), an A. aculeatus endoglucanase F1-CMC (Ooi et al. Nucleic Acid Res. 1990, 18:5884), an A. kawachii IFO 4308 endoglucanase CMCase-1 (Sakamoto et al. Curr. Genet. 1995, 27:435-439), an E. carotovara (Saarilahti et al. Gene 1990, 90:9-14); or an A. thermophilum ALK04245 endoglucanase (U.S. Patent Publication No. 20070148732) can also be used. Additional suitable endoglucanases are described in, e.g., WO 91/17243, WO 91/17244, WO 91/10732, U.S. Pat. No. 6,001,639.

Suitable polypeptides having GH61/endoglucanase activity are provided by the disclosure. In some embodiments, the polypeptide having GH61/endoglucanase activity is an EGIV polypeptide, e.g., a T. reesei Eg4 polypeptide. In some embodiments, the polypeptide is one having at least about 60% (e.g., at least about 60%, 65%, 70%, 75%, 80%, 85%, 90%, 91%, 92%, 93%, 94%, 95%, 96%, 97%, 98%, or 99%) sequence identity to any one of SEQ ID NOs: 52, 80-81, 206-207, over a region of at least about 10 (e.g., at least about 10, 15, 20, 25, 30, 35, 40, 45, 50, 55, 60, 65, 70, 75, 80, 85, 90, 95, 100, 125, 150, 175, 200, 225, 250, 275, 300) residues, or one that comprises one or more sequence motifs selected from the group consisting of: (1) SEQ ID NOs:84 and 88; (2) SEQ ID NOs:85 and 88; (3) SEQ ID NO:86; (4) SEQ ID NO:87; (5) SEQ ID NOs:84, 88 and 89; (6) SEQ ID NOs:85, 88, and 89; (7) SEQ ID NOs: 84, 88, and 90; (8) SEQ ID NOs: 85, 88 and 90; (9) SEQ ID NOs:84, 88 and 91; (10) SEQ ID NOs: 85, 88 and 91; (11) SEQ ID NOs: 84, 88, 89 and 91; (12) SEQ ID NOs: 84, 88, 90 and 91; (13) SEQ ID NOs: 85, 88, 89 and 91: and (14) SEQ ID NOs: 85, 88, 90 and 91. In certain embodiments, the composition further comprises a cellobiose dehydrogenase.

The GH61 endoglucanase(s) constitutes about 0.1 wt. % to about 50 wt. % of the total weight of enzymes in an enzyme blend/composition. The amount can be measured using known methods, including, e.g., SDS-PAGE, HPLC, or UPLC, as described in the Examples. The ratio of a pair of proteins relative to each other can be calculated based on these measurements. Blends/compositions comprising enzymes in any weight ratio derivable from the weight percentages herein are contemplated. The GH61 endoglucanase content can be in a range wherein the lower limit is about 0 wt. %, 1 wt. %, 2 wt. %, 3 wt. %, 4 wt. %, 5 wt. %, 6 wt. % 7 wt. %, 8 wt. %, 9 wt. %, 10 wt. %, 12 wt. %, 15 wt. %, 20 wt. %, 25 wt. %, 30 wt. %, 40 wt. %, 45 wt. % of the total weight of enzymes in the blend/composition, and the upper limit is about 10 wt,%, 15 wt,%, 16 wt. %, 20 wt. %, 25 wt. %, 30 wt. %, 35 wt. %, 40 wt. %, 50 wt. % of the total weight of enzymes in the blend/composition. For example, the GH61 endoglucanase(s) suitably represent about 2 wt. % to about 30 wt. %; about 8 wt. % to about 20 wt. %; about 3 wt. % to about 18 wt. %, about 4 wt. % to about 19 wt. %, or about 5 wt. % to about 20 wt. % of the total weight of enzymes in the blend/composition.

5.3.5.3. Cellobiohydrolases

Any cellobiohydrolase (EC 3.2.1.91) (“CBH”) can be optionally used in the methods and blends/compositions of the present disclosure. The cellobiohydrolase can be produced by expressing an endogeneous or exogeneous cellobiohydrolase gene. The cellobiohydrolase can be, in some circumstances, overexpressed or under expressed.

For example, T. reesei CBHI (Shoemaker et al. Bio/Technology 1983, 1:691-696) and/or CBHII (Teed et al. Bio/Technology 1983, 1:696-699) can be suitably used in the methods and blends/compositions of the present disclosure.

Suitable CBHs can be selected from an A. bisporus CBH1 (Swiss Prot Accession No. Q92400), an A. aculeatus CBH1 (Swiss Prot Accession No. 059843), an A. nidulans CBHA (GenBank Accession No. AF420019) or CBHB (GenBank Accession No. AF420020), an A. niger CBHA (GenBank Accession No. AF156268) or CBHB (GenBank Accession No. AF156269), a C. purpurea CBH1 (Swiss Prot Accession No. 000082), a C. carbonarum CBH1 (Swiss Prot Accession No. Q00328), a C. parasitica CBH1 (Swiss Prot Accession No. Q00548), a F. oxysporum CBH1 (Cel7A) (Swiss Prot Accession No. P46238), a H. grisea CBH1.2 (GenBank Accession No. U50594), a H. grisea var. thermoidea CBH1 (GenBank Accession No. D63515) a CBHI.2 (GenBank Accession No. AF123441), or an exol (GenBank Accession No. AB003105), a M. albomyces Cel7B (GenBank Accession No. AJ515705), a N. crassa CBHI (GenBank Accession No. X77778), a P. funiculosum CBHI (Cel7A) (U.S. Patent Publication No. 20070148730), a P. janthinellum CBHI (GenBank Accession No. S56178), a P. chrysosporium CBH (GenBank Accession No. M22220), or a CBHI-2 (Cel7D) (GenBank Accession No. L22656), a T. emersonii CBH1A (GenBank Accession No. AF439935), a T. viride CBH1 (GenBank Accession No. X53931), or a V. volvacea V14 CBH1 (GenBank Accession No. AF156693).

5.3.6. Whole Cellulases

An enzyme blend/composition of the disclosure can further comprise a whole cellulase. As used herein, a “whole cellulase” refers to either a naturally occurring or a non-naturally occurring cellulase-containing composition comprising at least 3 different enzyme types: (1) an endoglucanase, (2) a cellobiohydrolase, and (3) a β-glucosidase, or comprising at least 3 different enzymatic activities: (1) an endoglucanase activity, which catalyzes the cleavage of internal β-1,4 linkages, resulting in shorter glucooligosaccharides, (2) a cellobiohydrolase activity, which catalyzes an “exo”-type release of cellobiose units (β-1,4 glucose-glucose disaccharide), and (3) a β-glucosidase activity, which catalyzes the release of glucose monomer from short cellooligosaccharides (e.g., cellobiose).

A “naturally occurring cellulase-containing” composition is one produced by a naturally occurring source, which comprises one or more cellobiohydrolase-type, one or more endoglucanase-type, and one or more β-glucosidase-type components or activities, wherein each of these components or activities is found at the ratio and level produced in nature, untouched by the human hand. Accordingly, a naturally occurring cellulase-containing composition is, for example, one that is produced by an organism unmodified with respect to the cellulolytic enzymes such that the ratio or levels of the component enzymes are unaltered from that produced by the native organism in nature. A “non-naturally occurring cellulase-containing composition” refers to a composition produced by: (1) combining component cellulolytic enzymes either in a naturally occurring ratio or a non-naturally occurring, i.e., altered, ratio; or (2) modifying an organism to overexpress or underexpress one or more cellulolytic enzymes; or (3) modifying an organism such that at least one cellulolytic enzyme is deleted. A “non-naturally occurring cellulase containing” composition can also refer to a composition resulting from adjusting the culture conditions for a naturally-occurring organism, such that the naturally-occurring organism grows under a non-native condition, and produces an altered level or ratio of enzymes. Accordingly, in some embodiments, the whole cellulase preparation of the present disclosure can have one or more EGs and/or CBHs and/or β-glucosidases deleted and/or overexpressed.

A whole cellulase preparation may be from any microorganism capable of hydrolyzing a cellulosic material. For example, the whole cellulase preparation is a filamentous fungal whole cellulase. For example, the whole cellulase preparation can be from an Acremonium, Aspergillus, Emericella, Fusarium, Humicola, Mucor, Myceliophthora, Neurospora, Penicillium, Scytalidium, Thielavia, Tolypocladium, or Trichoderma species. The whole cellulase preparation is, example.g., an Aspergillus aculeatus, Aspergillus awamori, Aspergillus foetidus, Aspergillus japonicus, Aspergillus nidulans, Aspergillus niger, or Aspergillus oryzae whole cellulase. The whole cellulase preparation may be a Fusarium bactridioides, Fusarium cerealis, Fusarium crookwellense, Fusarium culmorum, Fusarium graminearum, Fusarium graminum, Fusarium heterosporum, Fusarium negundi, Fusarium oxysporum, Fusarium reticulatum, Fusarium roseum, Fusarium sambucinum, Fusarium sarcochroum, Fusarium sporotrichioides, Fusarium suiphureum, Fusarium torulosum, Fusarium trichothecioides, or Fusarium venenatum whole cellulase preparation. The whole cellulase preparation may also be a Humicola insolens, Humicola lanuginosa, Mucor miehei, Myceliophthora thermophila, Neurospora crassa, Penicillium purpurogenum, Penicillium funiculosum, Scytalidium thermophilum, Chrysosporium lucknowence or Thielavia terrestris whole cellulase preparation. Moreover, the whole cellulase preparation can be a Trichoderma harzianum, Trichoderma kiningii, Trichoderma longibrachiatum, Trichoderma reesei (e.g., RL-P37 (Sheir-Neiss G et al. Appl. Microbiol. Biotechnology, 1984, 20, pp. 46-53), QM9414 (ATCC No. 26921), NRRL 15709, ATCC 13631, 56764, 56466, 56767), or a Trichoderma viride (e.g., ATCC 32098 and 32086) whole cellulase preparation.

The whole cellulase preparation may, in particular, suitably be a T. reesei RutC30 whole cellulase preparation, which is available from the American Type Culture Collection as Trichoderma reesei ATCC 56765. For example, the whole cellulase preparation can also suitably be a whole cellulase of P. funiculosum, which is available from the American Type Culture Collection as P. funiculosum ATCC Number: 10446. Moreover, the whole cellulase preparation may be a bacterial whole cellulase prepration, e.g., one of a Bacillus or E. coli.

The whole cellulase preparation can also be obtained from commercial sources. Examples of commercial cellulase preparations suitable for use in the methods and compositions of the present disclosure include, for example, CELLUCLAST™ and Cellic™ (Novozymes A/S) and LAMINEX™ BG, IndiAge™ 44L, Primafast™ 100, Primafast™ 200, Spezyme™ CP, Accellerase® 1000 and Accellerase® 1500 (Danisco US. Inc., Genencor).

Whole cellulase preparations can be made using any known microorganism cultivation methods, resulting in the expression of enzymes capable of hydrolyzing a cellulosic material.

As used herein, “fermentation” refers to shake flask cultivation, small- or large-scale fermentation, such as continuous, batch, fed-batch, or solid state fermentations in laboratory or industrial fermenters performed in a suitable medium and under conditions that allow the cellulase and/or enzymes of interest to be expressed and/or isolated.

Generally, the microorganism is cultivated in a cell culture medium suitable for production of enzymes capable of hydrolyzing a cellulosic material. The cultivation takes place in a suitable nutrient medium comprising carbon and nitrogen sources and inorganic salts, using procedures and variations known in the art. Suitable culture media, temperature ranges and other conditions for growth and cellulase production are known. For example, a typical temperature range for production of cellulases by T. reesei is 24° C. to 28° C.

The whole cellulase preparation can be used as it is produced by fermentation with no or minimal recovery and/or purification. For example, once cellulases are secreted into the cell culture medium, the cell culture medium containing the cellulases can be used directly. The whole cellulase preparation can comprise the unfractionated contents of fermentation material, including the spent cell culture medium, extracellular enzymes and cells. On the other hand, the whole cellulase preparation can also be subject to further processing in a number of routine steps, e.g., precipitation, centrifugation, affinity chromatography, filtration, or the like. For example, the whole cellulase preparation can be concentrated, and then used without further purification. The whole cellulase preparation can, for example, be formulated to comprise certain chemical agents that decrease cell viability or kills the cells after fermentation. The cells can, for example, be lysed or permeabilized using methods known in the art.

The endoglucanase activity of the whole cellulase preparation can be determined using carboxymethyl cellulose (CMC) as a substrate. A suitable assay measures the production of reducing ends created by the enzyme mixture acting on CMC wherein 1 unit is the amount of enzyme that liberates 1 μmoL of product/min (Ghose, T. K., Pure & Appl. Chem. 1987, 59, pp. 257-268).

The whole cellulase can be a β-glucosidase-enriched cellulase. The β-glucosidase-enriched whole cellulase generally comprises a β-glucosidase and a whole cellulase preparation. The β-glucosidase-enriched whole cellulase compositions can be produced by recombinant means. For example, such a whole cellulase preparation can be achieved by expressing a β-glucosidase in a microorganism capable of producing a whole cellulase The β-glucosidase-enriched whole cellulase composition can also, for example, comprise a whole cellulase preparation and a β-glucosidase. Any of the β-glucosidase polypeptides described herein can be suitable, including, for example, one that is a chimeric/fusion β-glucosidase polypeptide. For instance, the β-glucosidase-enriched whole cellulase composition can suitably comprise at least about 5 wt. %, 7 wt. %, 9 wt. % 10 wt. %, or 14 wt. %, and up to about 17 wt. %, about 20 wt. %, 25 wt. %, 30 wt. %, 35 wt. %, 40 wt. %, or 50 wt. % β-glucosidase based on the total weight of proteins in that blend/composition.

5.3.7. Xylanases & β-Xylosidase

The enzyme blends/compositions of the disclosure, e.g., can, comprise one or more xylanases, which may be T. reesei Xyn2, T. reesei Xyn3, AfuXyn2, or AfuXyn5. Suitable T. reesei Xyn2, T. reesei Xyn3, AfuXyn2, or AfuXyn5 polypeptides are described herein.

The enzyme blends/compositions of the disclosure optionally comprise one or more xylanases in addition to or in place of the one or more xylanases. Any xylanase (EC 3.2.1.8) may be used as the additional one or more xylanases. Suitable xylanases include, e.g., a C. saccharolyticum xylanase (Luthi et al. 1990, Appl. Environ. Microbiol. 56(9):2677-2683), a T. maritima xylanase (Winterhalter & Liebel, 1995, Appl. Environ. Microbiol. 61(5):1810-1815), a Thermatoga Sp. Strain FJSS-B.1 xylanase (Simpson et al. 1991, Biochem. J. 277, 413-417), a B. circulans xylanase (BcX) (U.S. Pat. No. 5,405,769), an A. niger xylanase (Kinoshita et al. 1995, Journal of Fermentation and Bioengineering 79(5):422-428), a S. lividans xylanase (Shareck et al. 1991, Gene 107:75-82; Morosoli et al. 1986 Biochem. J. 239:587-592; Kluepfel et al. 1990, Biochem. J. 287:45-50), a B. subtilis xylanase (Bernier et al. 1983, Gene 26(1):59-65), a C. fimi xylanase (Clarke et al., 1996, FEMS Microbiology Letters 139:27-35), a P. fluorescens xylanase (Gilbert et al. 1988, Journal of General Microbiology 134:3239-3247), a C. thermocellum xylanase (Dominguez et al., 1995, Nature Structural Biology 2:569-576), a B. pumilus xylanase (Nuyens et al. Applied Microbiology and Biotechnology 2001, 56:431-434; Yang et al. 1998, Nucleic Acids Res. 16(14B):7187), a C. acetobutylicum P262 xylanase (Zappe et al. 1990, Nucleic Acids Res. 18(8):2179), or a T. harzianum xylanase (Rose et al. 1987, J. Mol. Biol. 194(4):755-756).

The xylanase can be produced by expressing an endogenous or exogenous gene encoding a xylanase. The xylanase may be, for example, overexpressed or underexpressed. The enzyme blends/compositions of the disclosure, e.g., can suitablycomprise one or more β-xylosidases. For example, the β-xylosidase is a Group 1 β-xylosidase enzyme (e.g., Fv3A or Fv43A) or a Group 2 β-xylosidase enzyme (e.g., Pf43A, Fv43D, Fv39A, Fv43E, Fo43A, Fv43B, Pa51A, Gz43A, or T. reesei Bxl1). For example, an enzyme blend/composition of the disclosure can suitably comprise one or more Group 1 β-xylosidases and one or more Group 2 β-xylosidases.

The enzyme blends/compositions of the disclosure can optionally comprise one or more β-xylosidases, in addition to or in place of the Group 1 and/or Group 2 β-xylosidases above. Any β-xylosidase (EC 3.2.1.37) can be used as the additional β-xylosidases. Suitable β-xylosidases include, e.g., a T. emersonii Bxl1 (Reen et al. 2003, Biochem Biophys Res Commun. 305(3):579-85), a G. stearothermophilus β-xylosidases (Shallom et al. 2005, Biochemistry 44:387-397), a S. thermophilum β-xylosidases (Zanoelo et al. 2004, J. Ind. Microbiol. Biotechnol. 31:170-176), a T. lignorum β-xylosidases (Schmidt, 1998, Methods Enzymol. 160:662-671), an A. awamori β-xylosidases (Kurakake et al. 2005, Biochim. Biophys. Acta 1726:272-279), an A. versicolor β-xylosidases (Andrade et al. 2004, Process Biochem. 39:1931-1938), a Streptomyces sp. β-xylosidases (Pinphanichakarn et al. 2004, World J. Microbiol. Biotechnol. 20:727-733), a T. maritima β-xylosidases (Xue and Shao, 2004, Biotechnol. Lett. 26:1511-1515), a Trichoderma sp. SY β-xylosidases (Kim et al. 2004, J. Microbiol. Biotechnol. 14:643-645), an A. niger β-xylosidases (Oguntimein and Reilly, 1980, Biotechnol. Bioeng. 22:1143-1154), or a P. wortmanni β-xylosidases (Matsuo et al. 1987, Agric. Biol. Chem. 51:2367-2379).

The β-xylosidase can be produced by expressing an endogenous or exogenous gene encoding a β-xylosidase. The β-xylosidase can be, in some circumstances, overexpressed or underexpressed.

5.3.8. L-α-Arabinofuranosidases

The enzyme blends/compositions of the disclosure can, for example, suitably comprise one or more L-α-arabinofuranosidases. The L-α-arabinofuranosidase is, e.g., Af43A, Fv43B, Pf51A, Pa51A, Fv51A, Af43A, Fv43B, Pf51A, Pa51A, or Fv51A polypeptide. The enzyme blends/compositions of the disclosure optionally comprise one or more L-α-arabinofuranosidases in addition to or in place of the foregoing L-α-arabinofuranosidases. L-α-arabinofuranosidases (EC 3.2.1.55) from any suitable organism can be used as the additional L-α-arabinofuranosidases. Suitable L-α-arabinofuranosidases include, e.g., an L-α-arabinofuranosidases of A. oryzae (Numan & Bhosle, J. Ind. Microbiol. Biotechnol. 2006, 33:247-260), A. sojae (Oshima et al. J. Appl. Glycosci. 2005, 52:261-265), B. brevis (Numan & Bhosle, J. Ind. Microbiol. Biotechnol. 2006, 33:247-260), B. stearothermophilus (Kim et al., J. Microbiol. Biotechnol. 2004,14:474-482), B. breve (Shin et al., Appl. Environ. Microbiol. 2003, 69:7116-7123), B. longum (Margolles et al., Appl. Environ. Microbiol. 2003, 69:5096-5103), C. thermocellum (Taylor et al., Biochem. J. 2006, 395:31-37), F. oxysporum (Panagiotou et al., Can. J. Microbiol. 2003, 49:639-644), F. oxysporum f. sp. dianthi (Numan & Bhosle, J. Ind. Microbiol. Biotechnol. 2006, 33:247-260), G.stearothermophilus T-6 (Shallom et al., J. Biol. Chem. 2002, 277:43667-43673), H. vulgare (Lee et al., J. Biol. Chem. 2003, 278:5377-5387), P. chrysogenum (Sakamoto et al., Biophys. Acta 2003, 1621:204-210), Penicillium sp. (Rahman et al., Can. J. Microbiol. 2003, 49:58-64), P. cellulosa (Numan & Bhosle, J. Ind. Microbiol. Biotechnol. 2006, 33:247-260), R. pusillus (Rahman et al., Carbohydr. Res. 2003, 338:1469-1476), S. chartreusis, S. thermoviolacus, T. ethanolicus, T. xylanilyticus (Numan & Bhosle, J. Ind. Microbiol. Biotechnol. 2006, 33:247-260), T. fusca (Tuncer and Ball, Folia Microbiol. 2003, (Praha) 48:168-172), T. maritima (Miyazaki, Extremophiles 2005, 9:399-406), Trichoderma sp. SY (Jung et al. Agric. Chem. Biotechnol. 2005, 48:7-10), A. kawachii (Koseki et al., Biochim. Biophys. Acta 2006, 1760:1458-1464), F. oxysporum f. sp. dianthi (Chacon-Martinez et al., Physiol.Mol. Plant Pathol. 2004, 64:201-208), T. xylanilyticus (Debeche et al., Protein Eng. 2002, 15:21-28), H. insolens, M. giganteus (Sorensen et al., Biotechnol. Prog. 2007, 23:100-107), or R. sativus (Kotake et al. J. Exp. Bot. 2006, 57:2353-2362).

The L-α-arabinofuranosidase can be produced by expressing an endogenous or exogenous gene encoding an L-α-arabinofuranosidase. The L-α-arabinofuranosidase can be, in some circumstances, overexpressed or underexpressed.

5.3.9. Cellobiose Dehydrogenases

The term “cellobiose dehydrogenase” refers to an oxidoreductase of E.C. 1.1.99.18 that catalyzes the conversion of cellobiose in the presence of an acceptor to cellobiono-1,5-lactone and a reduced acceptor. 2,6-Dichloroindophenol, like iron, molecule oxygen, ubiquinone, or cytochrome C, or another polyphenol, can act as an acceptor. Substrates of cellobiose dehydrogenase include, without limitation, cellobiose, cello-oligosaccharides, lactose, and D-glucosyl-1,4-β-D-mannose, glucose, maltose, mannobiose, thiocellobiose, galactosyl-mannose, xylobiose, and xylose. Electron donors include, β-1-4 dihexoses with glucose or mannose at the reducing end, α-1-4-hexosides, hexoses, pentoses, and β-1-4-pentomers. See, Henriksson et al., 1998, Biochimica et Biophysica Acta—Protein Structure and Molecular Enzymology, 1383:48-54; Schou et al., 1998, Biochem. J. 330:565-571.

Two families of cellobiose dehydrogenases may be suitably included in an enzyme composition of the present disclosure or be expressed by an engineered host cell herein, family 1 and family 2. The two families are differentiated by the presence of a cellulose binding motif (CBM) in family 1 but not in family 2. The 3-dimensional structure of cellobiose dehydrogeanase indicates two globular domains, each containing one of the two co-factors: a heme or a flavin. The active site lies at a cleft between the two domains. The catalytic cycle of cellobiose dehydrogenase follows an ordered sequential mechanism. Oxidation of cellobiose occurs by a 2-electron transfer from cellobiose to the flavin, generating cellobiono-1,5-lactone and reduced flavin. The active FAD is then regenerated by electron transfer to the heme group, leaving a reduced heme. The native state heme is regenerated by reaction with the oxidizing substrate at the second active site.

The oxidizing subsgtrate can be iron ferrcyanide, cytochrome C, or an oxidized phenolic compound, e.g., dichloroindophenol (DCIP), a common substrate used in colormetric assays. Metal ions and O₂ are also suitably substrates to these enzymes, although the reaction rate of cellobiose dehydrogenases are substantially lower with regard to these substrates as compared to when iron or organic oxidants are used as substrates. After cellobionolactone is released, the product can undergo spontaneous ring-opening to generate cellobionic acid. See, Hallberg et al., 2003, J. Biol. Chem. 278:7160-66.

5.3.10. Other Components

The engineered enzyme compositions of the disclosure can, e.g., suitably further comprise one or more accessory proteins. Examples of accessory proteins include, without limitation, mannanases (e.g., endomannanases, exomannanases, and β-mannosidases), galactanases (e.g., endo- and exo-galactanases), arabinases (e.g., endo-arabinases and exo-arabinases), ligninases, amylases, glucuronidases, proteases, esterases (e.g., ferulic acid esterases, acetyl xylan esterases, coumaric acid esterases or pectin methyl esterases), lipases, other glycoside hydrolases, xyloglucanases, CIP1, CIP2, swollenins, expansins, and cellulose disrupting proteins. In particular embodiments, the cellulose disrupting proteins are cellulose binding modules.

5.4. Methods & Processes

The disclosure thus further provides a process of saccharification a biomass material comprising hemicelluloses, and optionally comprising cellulose. Exemplary biomass materials include, without limitation, corcob, switchgrass, sorghum, and/or bagasse. Accordingly the disclosure provides a process of saccharification, comprising treating a biomass material herein comprising hemicelluose and optionally cellose with an enzyme blend/composition as described herein. The enzyme blend/composition used in such a process of the invention include 1 g to 40 g (e.g., 2 g to 20 g, 3 g to 7 g, 1 g to 5 g, or 2 g to 5 g) of polypeptides having xylanase activity per kg of hemicellulose in the biomass material.

The enzyme blend/composition used in such a process can also include 1 g to 50 g (e.g., 2 g to 40 g, 4 g to 20 g, 4 g to 10 g, 2 g to 10 g, 3 g to 7 g) of polypeptide having β-xylosidase activity per kg of hemicellulose in the biomass material. The enzyme blend/composition used in such a process of the invention can include 0.5 g to 20 g (e.g., 1 g to 10 g, 1 g to 5 g, 2 g to 6 g, 0.5 g to 4 g, or 1 g to 3 g) of polypeptides having L-α-arabinofuranosidase activity per kg of hemicellulose in the biomass material. The enzyme blend/composition can also include 1 g to 100 g (e.g., 3 g to 50 g, 5 g to 40 g, 10 g to 30 g, or 12 g to 18 g) of polypeptides having cellulase activity per kg of cellulose in the biomass material.

Optionally, the amount of polypeptides having β-glucosidase activity constitutes up to 50% of the total weight of polypeptides having cellulase activity.

A suitable process of the invention preferably yields 60% to 90% xylose from the hemicellulose xylan of the biomass material treated. Suitable biomass materials include one or more of, e.g., corncob, switchgrass, sorghum, and/or bagasse. As suich, a process of the invention preferably yields at least 70% (e.g., at least 75%, at least 80%) xylose from hemicellulose xylan from one or more of these biomass materials. For example, the process yields 60% to 90% of xylose from hemicellulose xylan of a biomass material comprising hemicellulose, including, without limitation, corncob, switchgrass, sorghum, and/or bagasse.

The process of the invention optionally further comprises recovering monosaccharides. In addition to saccharification of biomass, the enzymes and/or enzyme blends of the disclosure can be used in industrial, agricultural, food and feed, as well as food and feed supplement processing processes. Examples of applications are described below.

5.4.1. Wood, Paper and Pulp Treatments

The enzymes, enzyme blends/compositions, and methods of the disclosure can be used in wood, wood product, wood waste or by-product, paper, paper product, paper or wood pulp, Kraft pulp, or wood or paper recycling treatment or industrial process. These processes include, e.g., treatments of wood, wood pulp, paper waste, paper, or pulp, or deinking of wood or paper. The enzymes, enzyme blends/compositions of the disclosure can be, e.g., used to treat/pretreat paper pulp, or recycled paper or paper pulp, and the like. The enzymes, enzyme blends/compositions of the disclosure can be used to increase the “brightness” of the paper when they are included in the paper, pulp, recycled paper or paper pulp treatment/pretreatment. It can be appreciated that the higher the grade of paper, the greater the brightness; the brightness can impact the scan capability of optical scanning equipment. As such, the enzymes, enzyme blends/compositions, and mthods/processes can be used to make high grade, “bright” papers, including inkjet, laser and photo printing quality paper.

The enzymes, enzyme blends/compositions of the disclosure can be used to process or treat a number of other cellulosic material, including, e.g., fibers from wood, cotton, hemp, flax or linen.

Accordingly, the disclosure provides wood, wood pulp, paper, paper pulp, paper waste or wood or paper recycling treatment processes using an enzyme, enzyme blend/composition of the disclosure.

The enzymes, enzyme blends/compositions of the disclosure can be used for deinking printed wastepaper, such as newspaper, or for deinking noncontact-printed wastepaper, e.g., xerographic and laser-printed paper, and mixtures of contact and noncontact-printed wastepaper, as described in U.S. Pat. No. 6,767,728 or 6,426,200; Neo, J. Wood Chem. Tech. 1986, 6(2):147. They can also be used to produce xylose from a paper-grade hardwood pulp in a process involving extracting xylan contained in pulp into a liquid phase, subjecting the xylan contained in the obtained liquid phase to conditions sufficient to hydrolyze xylan to xylose, and recovering the xylose. The extracting step, e.g., can include at least one treatment of an aqueous suspension of pulp or an alkali-soluble material by an enzyme or an enzyme blend/composition (see, U.S. Pat. No. 6,512,110). The enzymes, enzyme blends/compositions of the disclosure can be used to dissolve pulp from cellulosic fibers such as recycled paper products made from hardwood fiber, a mixture of hardwood fiber and softwood fiber, waste paper, e.g., from unprinted envelopes, de-inked envelopes, unprinted ledger paper, de-inked ledger paper, and the like, as described in, e.g., U.S. Pat. No. 6,254,722.

5.4.2. Treating Fibers and Textiles

The disclosure provides methods of treating fibers and fabrics using one or more enzymes, enzyme blends/compositions of the disclosure. The enzymes, enzyme blends/compositions can be used in any fiber- or fabric-treating method, which are known in the art. See, e.g., U.S. Pat. Nos. 6,261,828; 6,077,316; 6,024,766; 6,021,536; 6,017,751; 5,980,581; U.S. Patent Publication No. 20020142438 A1. For example, enzymes, enzyme blends/compositions of the disclosure can be used in fiber and/or fabric desizing. The feel and appearance of a fabric can be, e.g., improved by a method comprising contacting the fabric with an enzyme or enzyme blend/composition of the disclosure in a solution.

Optionally, the fabric is treated with the solution under pressure. The enzymes, enzyme blends/composition of the disclosure can also be used to remove stains.

The enzymes, enzyme blends/compositions of the disclosure can be used to treat a number of other cellulosic material, including fibers (e.g., fibers from cotton, hemp, flax or linen), sewn and unsewn fabrics, e.g., knits, wovens, denims, yarns, and toweling, made from cotton, cotton blends or natural or manmade cellulosics or blends thereof. The textile treating processes can be used in conjunction with other textile treatments, e.g., scouring and/or bleaching. Scouring, e.g., is the removal of non-cellulosic material from the cotton fiber, e.g., the cuticle (mainly consisting of waxes) and primary cell wall (mainly consisting of pectin, protein and xyloglucan).

5.4.3. Treating Foods and Food Processing

The enzymes, enzyme blends/compositions of the disclosure have numerous applications in food processing industry. They can, e.g., be used to improve extraction of oil from oil-rich plant material, e.g., oil-rich seeds. The enzymes, enzyme blends/compositions of the disclosure can be used to extract soybean oil from soybeans, olive oil from olives, rapeseed oil from rapeseed, or sunflower oil from sunflower seeds.

The enzymes, enzyme blends/compositions of the disclosure can also be used to separate components of plant cell materials. For example, they can be used to separate plant cells into components. The enzymes, enzyme blends/compositions of the disclosure can also be used to separate crops into protein, oil, and hull fractions. The separation process can be performed using known methods.

The enzymes, enzyme blends/compositions of the disclosure can, in addition to the uses above, be used to increase yield in the preparation of fruit or vegetable juices, syrups, extracts and the like. They can also be used in the enzymatic treatment of various plant cell wall-derived materials or waste materials from, e.g., cereals, grains, wine or juice production, or agricultural residues such as, e.g., vegetable hulls, bean hulls, sugar beet pulp, olive pulp, potato pulp, and the like. Further, they can be used to modify the consistency and/or appearance of processed fruits or vegetables. They can also be used to treat plant material so as to facilitate processing of the plant material (including foods), purification or extraction of plant components. The enzymes and blends/compositions of the disclosure can be used to improve feed value, decrease the water binding capacity, improve the degradability in waste water plants and/or improve the conversion of plant material to ensilage, and the like.

The enzymes, enzyme blends/compositions herein can be used in baking applications. For exaxmple, they are used to create non-sticky doughs that are not difficult to machines and to reduce biscuit sizes. They are also used to hydrolyze arabinoxylans to prevent rapid rehydration of the baked product that can lead to loss of crispiness and reduced shelf-life. For example they are used as additives in dough processing.

5.4.4. Animal Feeds and Food or Feed or Food Additives

Provided are methods for treating animal feeds/foods and food or feed additives (supplements) using enzymes, and blends/compositions of the disclosure. Animals including mammals (e.g., humans), birds, fish, and the like. The disclosure provides animal feeds, foods, and additives (supplements) comprising enzymes and enzyme blends/compositions of the disclosure. Treating animal feeds, foods and additives using the enzymes can add to the availability of nutrients, e.g., starch, protein, and the like, in the animal feed or additive (supplements). By breaking down difficult-to-digest proteins or indirectly or directly unmasking starch (or other nutrients), the enzymes and blends/compositions can make nutrients more accessible to other endogenous or exogenous enzymes. They can also simply cause the release of readily digestible and easily absorbed nutrients and sugars. When added to animal feed, enzymes, enzyme blends/compositions of the disclosure improve the in vivo break-down of plant cell wall material partly by reducing the intestinal viscosity (see, e.g., Bedford et al., Proceedings of the 1st Symposium on Enzymes in Animal Nutrition, 1993, pp. 73-77), whereby a better utilization of the plant nutrients by the animal is achieved. Thus, by using enzymes, enzyme blends/compositions of the disclosure in feeds, the growth rate and/or feed conversion ratio (i.e., the weight of ingested feed relative to weight gain) of the animal can be improved.

The animal feed additive of the disclosure may be a granulated enzyme product which can be readily mixed with feed components. Alternatively, feed additives of the disclosure can form a component of a pre-mix. The granulated enzyme product of the disclosure may be coated or uncoated. The particle size of the enzyme granulates can be compatible with that of the feed and/or the pre-mix components. This provides a safe and convenient mean of incorporating enzymes into feeds. Alternatively, the animal feed additive of the disclosure can be a stabilized liquid composition. This may be an aqueous- or oil-based slurry. See, e.g., U.S. Pat. No. 6,245,546.

An enzyme, enzyme blend/composition of the disclosure can be supplied by expressing the enzymes directly in transgenic feed crops (e.g., as transgenic plants, seeds and the like), such as grains, cereals, corn, soy bean, rape seed, lupin and the like. As discussed above, the disclosure provides transgenic plants, plant parts and plant cells comprising a nucleic acid sequence encoding a polypeptide of the disclosure. The nucleic acid is expressed such that the enzyme of the disclosure is produced in recoverable quantities. The xylanase can be recovered from any plant or plant part. Alternatively, the plant or plant part containing the recombinant polypeptide can be used as such for improving the quality of a food or feed, e.g., improving nutritional value, palatability, and rheological properties, or to destroy an antinutritive factor.

The disclosure provides methods for removing oligosaccharides from feed prior to consumption by an animal subject using an enzyme, enzyme blend/composition of the disclosure. In this process a feed is formed to have an increased metabolizable energy value. In addition to enzymes, enzyme blends/compositions of the disclosure, galactosidases, cellulases, and combinations thereof can be used.

The disclosure provides methods for utilizing an enzyme, an enzyme blend/composition of the disclosure as a nutritional supplement in the diets of animals by preparing a nutritional supplement containing a recombinant enzyme of the disclosure, and administering the nutritional supplement to an animal to increase the utilization of hemicellulase contained in food ingested by the animal.

5.4.5 Waste Treatment

The enzymes, enzyme blends/compositions of the disclosure can be used in a variety of other industrial applications, e.g., in waste treatment. For example, in one aspect, the disclosure provides solid waste digestion process using the enzymes, enzyme blends/compositions of the disclosure. The methods can comprise reducing the mass and volume of substantially untreated solid waste. Solid waste can be treated with an enzymatic digestive process in the presence of an enzymatic solution (including the enzymes, enzyme blends/compositions of the disclosure) at a controlled temperature. This results in a reaction without appreciable bacterial fermentation from added microorganisms. The solid waste is converted into a liquefied waste and residual solid waste. The resulting liquefied waste can be separated from said any residual solidified waste. See, e.g., U.S. Pat. No. 5,709,796.

5.4.6 Detergent, Disinfectant and Cleaning Compositions

The disclosure provides detergent, disinfectant or cleanser (cleaning or cleansing) compositions comprising one or more enzymes, enzyme blends/compositions of the disclosure, and methods of making and using these compositions. The disclosure incorporates all known methods of making and using detergent, disinfectant or cleanser compositions. See, e.g., U.S. Pat. Nos. 6,413,928; 6,399,561; 6,365,561; 6,380,147.

In specific embodiments, the detergent, disinfectant or cleanser compositions can be a one- and two-part aqueous composition, a non-aqueous liquid composition, a cast solid, a granular form, a particulate form, a compressed tablet, a gel and/or a paste and a slurry form. The enzymes, enzyme blends/compositions of the disclosure can also be used as a detergent, disinfectant, or cleanser additive product in a solid or a liquid form. Such additive products are intended to supplement or boost the performance of conventional detergent compositions, and can be added at any stage of the cleaning process.

The present disclosure provides cleaning compositions including detergent compositions for cleaning hard surfaces, for cleaning fabrics, dishwashing compositions, oral cleaning compositions, denture cleaning compositions, and contact lens cleaning solutions. When the enzymes of the disclosure are components of compositions suitable for use in a laundry machine washing method, the compositions can comprise, in addition to an enzyme, enzyme blend/composition of the disclosure, a surfactant and a builder compound. They can additionally comprise one or more detergent components, e.g., organic polymeric compounds, bleaching agents, additional enzymes, suds suppressors, dispersants, lime-soap dispersants, soil suspension and anti-redeposition agents, and corrosion inhibitors. Laundry compositions of the disclosure can also contain softening agents, as additional detergent components. Such compositions containing carbohydrase can provide fabric cleaning, stain removal, whiteness maintenance, softening, color appearance, dye transfer inhibition and sanitization when formulated as laundry detergent compositions.

5.4.7. Industrial, Commercial, and Business Methods

The cellulase and/or hemicellulase compositions of the disclosure can be further used in industrial and/or commercial settings. Accordingly a method or a method of manufacturing, marketing, or otherwise commercializing the instant non-naturally occurring cellulase and/or hemicellulase compositions is also contemplated.

In a specific embodiment, the cellulase polypeptides, including, e.g., the endoglucanase polypeptides (e.g., the GH61 endoglucanases, such as T. reesei Eg4 polypeptide), the β-glucosidase polypeptides (e.g., the Pa3D, Fv3G, Fv3D, Fv3C, Tr3A, Tr3B, Te3A, An3A, Fo3A, Gz3A, Nh3A, Vd3A, Pa3G, and Tn3B polypeptides herein, the polypeptide having at least about 60% sequence identity to any one of SEQ ID NOs: 54, 56, 58, 60, 62, 64, 66, 68, 70, 72, 74, 76, 78, and 79, and/or the fusion/chimeric polypeptide comprising at least two β-glucosidase sequences, wherein the first β-glucosidase sequence is one of at least about 200 amino acid residues in length and comprises one or more or all of SEQ ID NOs:96-108, whereas the second β-glucosidase sequence is one of at least about 50 amino acid residues in length and comprises one or more or all of SEQ ID NOs:109-116), the cellobiohydrolase polypeptides, and the hemicellulase polypeptides, including the β-xylosidase polypeptides, the xylanase polypeptides, and the L-α-arabinofuranosidase polypeptides, as well as the cellulase compositions and/or hemicellulase compositions comprising the above-mentioned polypeptides can be supplied or sold to certain ethanol (bioethanol) refineries or other bio-chemical or bio-material manufacturers. In a first example, the non-naturally occurring cellulase and/or hemicellulase compositions can be manufactured in an enzyme manufacturing facility that is specialized in manufacturing enzymes at an industrial scale.

The non-naturally occurring cellulase and/or hemicellulase compositions can then be packaged or sold to customers of the enzyme manufacturer. This operational strategy is termed the “merchant enzyme supply model” herein.

In another operational strategy, the non-naturally occurring cellulase and hemicellulase compositions of the invention can be produced in a state of the art enzyme production system that is built by the enzyme manufacturer at a site that is located at or in the vicinity of the bioethanol refineries or the bio-chemical/biomaterial manufacturers (“on-site”). In some embodiments, an enzyme supply agreement is executed by the enzyme manufacturer and the bioethanol refineries or the bio-chemical/biomaterial manufacturer. The enzyme manufacturer designs, controls and operates the enzyme production system on site, utilizing the host cell, expression, and production methods as described herein to produce the non-naturally-occurring cellulase and/or hemicellulase compositions. In certain embodiments, suitable biomass, preferably subject to appropriate pretreatments as described herein, can be hydrolyzed using the saccharification methods and the enzymes and/or enzyme compositions herein at or near the bioethanol refineries or the bio-chemical/biomaterial manufacturing facilities. The resulting fermentable sugars can then be subject to fermentation at the same facilities or at facilities in the vicinity. This operational strategy is termed the “on-site biorefinery model” herein.

The on-site biorefinery model provides certain advantages over the merchant enzyme supply model, including, e.g., the provision of a self-sufficient operation, allowing minimal reliance on enzyme supply from merchant enzyme suppliers. This in turn allows the bioethanol refineries or the bio-chemical/biomaterial manufacturers to better control enzyme supply based on real-time or nearly real-time demand. In certain embodiments, it is contemplated that an on-site enzyme production facility can be shared between two, or among two or more bioethanol refineries and/or the bio-chemical/biomaterial manufacturers located near to each other, reducing the cost of transporting and storing enzymes. Further, this allows more immediate “drop-in” technology improvements at the enzyme production facility on-site, reducing the time lag between the improvements of enzyme compositions to a higher yield of fermentable sugars and ultimately, bioethanol or biochemicals.

The on-site biorefinery model has more general applicability in the industrial production and commercialization of bioethanols and biochemicals, as it may be used to manufacture, supply, and produce not only the cellulase and non-naturally occurring hemicellulase compositions herein but also the enzymes and enzyme compositions that process starch (e.g., corn) to allow for more efficient and effective direct conversion of starch to bioethanol/bio-chemicals. The starch-processing enzymes can, in certain embodiments, be produced in the on-site biorefinery, and then easily integrated into the bioethanol refinery or the biochemical/biomaterial manufacturing facility in order to produce bioethanol.

Thus in certain aspects, the invention also pertains to certain business methods of applying the enzymes (e.g., certain β-glucosidase polypeptides (including variants, mutants or chimeric polypeptides), and certain GH61 endoglucanases (including variants, mutants and the like), cells, compositions, and processes herein in the manufacturing and marketing of certain bioethanol, biofuel, biochemicals or other biomaterials. In some embodiments, the invention pertains to the application of such enzymes, cells, compositions and processes in an on-site biorefinery model. In other embodiments, the invention pertains to the application of such enzymes, cells, compositions and processes in a merchant enzyme supply model.

### 6. EXAMPLES

### 6.1 Example 1

### Assays/Methods

The following assays/methods were generally used in the Examples described below. Any deviations from the protocols provided below are indicated in specific Examples.

6.1.1. A. Pretreatment of Biomass Substrates

Corncob, corn stover and switch grass were pretreated prior to enzymatic hydrolysis according to the methods and processing ranges described in WO06110901A (unless otherwise noted). These references for pretreatment are also included in the disclosures of US-2007-0031918-A1, US-2007-0031919-A1, US-2007-0031953-A1, and/or US-2007-0037259-A1.

Ammonia fiber explosion treated (AFEX) corn stover was obtained from Michigan Biotechnology Institute International (MBI). The composition of the corn stover was determined using the National Renewable Energy Laboratory (NREL) procedure, NREL LAP-002 (Teymouri, F et al. Applied Biochemistry and Biotechnology, 2004, 113:951-963). NREL procedures are available at: http://www.nrel.gov/biomass/analytical_procedures.html. The FPP pulp and paper substrates were obtained from SMURFIT KAPPA CELLULOSE DU PIN, France.

Steam Expanded Sugar-cane Bagasse (SEB) was obtained from SunOpta (Glasser, W G et al. Biomass and Bioenergy 1998, 14(3): 219-235; Jollez, P et al. Advances in thermochemical biomass conversion, 1994, 2:1659-1669).

6.1.2. B. Compositional Analysis of Biomass

The 2-step acid hydrolysis method described in Determination of structural carbohydrates and lignin in the biomass (National Renewable Energy Laboratory, Golden, Colo. 2008 http://www.nrel.gov/biomass/pdfs/42618.pdf) was used to measure the composition of biomass substrates. Using this method, enzymatic hydrolysis results were reported herein in terms of percent conversion with respect to the theoretical yield from the starting glucan and xylan content of the substrate.

6.1.3. C. Total Protein Assay

The BCA protein assay is a colorimetric assay that measures protein concentration with a spectrophotometer. The BCA Protein Assay Kit (Pierce Chemical, Product #23227) was used according to the manufacturer's suggestion. Enzyme dilutions were prepared in test tubes using 50 mM sodium acetate pH 5 buffer. Diluted enzyme solution (0.1 mL) was added to 2 mL Eppendorf centrifuge tubes containing 1 mL 15% tricholoroacetic acid (TCA). The tubes were vortexed and placed in an ice bath for 10 min. The samples were then centrifuged at 14000 rpm for 6 min. The supernatant was poured out, the pellet was resuspended in 1 mL 0.1 N NaOH, and the tubes vortexed until the pellet dissolved. BSA standard solutions were prepared from a stock solution of 2 mg/mL. BCA working solution was prepared by mixing 0.5 mL Reagent B with 25mL Reagent A. 0.1 mL of the enzyme resuspended sample was added to 3 Eppendorf centrifuge tubes. Two mL Pierce BCA working solution was added to each sample and BSA standard Eppendorf tubes. All tubes were incubated in a 37° C. waterbath for 30 min. The samples were then cooled to room temperature (15 min) and the absorbance measured at 562 nm in a spectrophotometer. Average values for the protein absorbance for each standard were calculated. The average protein standard was plotted, absorbance on x-axis and concentration (mg/mL) on the y-axis. The points were fit to a linear equation:

y=mx+b

The raw concentration of the enzyme samples was calculated by substituting the absorbance for the x-value. The total protein concentration was calculated by multiplying with the dilution factor.

The total protein of purified samples was determined by A280 (Pace, C N, et al. Protein Science, 1995, 4:2411-2423).

Some protein samples were measured using the Biuret method as modified by Weichselbaum and Gornall using Bovine Serum Albumin as a calibrator (Weichselbaum, T. Amer. J. Clin. Path. 1960, 16:40; Gornall, A. et al. J. Biol. Chem. 1949, 177:752).

The total protein content of fermentation products was sometimes measured as total nitrogen by combustion, capture and measurement of released nitrogen, either by Kjeldahl (rtech laboratories, www.rtechlabs.com) or in-house by the DUMAS method (TruSpec C N, www.leco.com) (Sader, A. P. O. et al., Archives of Veterinary Science, 2004, 9(2):73-79). For complex protein-containing samples, e.g. fermentation broths, an average 16% N content, and the conversion factor of 6.25 for nitrogen to protein was used. In some cases, total precipitable protein was measured to remove interfering non-protein nitrogen. A 12.5% final TCA concentration was used and the protein-containing TCA pellet was resuspended in 0.1 M NaOH.

In some cases, Coomassie Plus—the Better Bradford Assay (Thermo Scientific, Rockford, Ill. product #23238) was used according to manufacturer recommendation.

6.1.4 D. Glucose Determination Using ABTS

The ABTS (2,2′-azino-bis(3-ethylenethiazoline-6)-sulfonic acid) assay for glucose determination was based on the principle that in the presence of O₂, glucose oxidase catalyzes the oxidation of glucose while producing stoichiometric amounts of hydrogen peroxide (H₂O₂). This reaction is followed by a horse radish peroxidase (HRP)-catalyzed oxidation of ABTS, which linearly correlates to the concentration of H₂O₂. The emergence of oxidized ABTS is indicated by the evolution of a green color, which is quantified at an OD of 405 nm. A mixture of 2.74 mg/mL ABTS powder (Sigma), 0.1 U/mL HRP (Sigma) and 1 U/mL Glucose Oxidase, (OxyGO® HP L5000, Genencor, Danisco USA) was prepared in a 50 mM sodium acetate buffer, pH 5.0, and kept in the dark. Glucose standards (at 0, 2, 4, 6, 8, 10 nmol) were prepared in 50 mM sodium acetate Buffer, pH 5.0. Ten (10) μL of the standards was added individually to a 96-well flat bottom micro titer plate in triplicate. Ten (10) μL of serially diluted samples were also added to the plate. One hundred (100) μL of ABTS substrate solution was added to each well and the plate was placed on a spectrophotometric plate reader. Oxidation of ABTS was read for 5 min at 405 nm.

Alternately, the ODs at 405 nm of the samples were measured after 15-30 min of incubation followed by quenching of the reaction using a quenching mix containing 50 mM sodium acetate buffer, pH 5.0, and 2% SDS.

6.1.5. E. Sugar Analysis by HPLC

Samples from cob saccharification hydrolysis were prepared by removing insoluble material using centrifugation, filtration through a 0.22 μm nylon Spin-X centrifuge tube filter (Corning, Corning, N.Y.), and dilution to the desired concentrations of soluble sugars using distilled water. Monomer sugars were determined on a Shodex Sugar SH-G SH1011, 8×300 mm with a 6×50 mm SH-1011P guard column (www.shodex.net). The solvent used was 0.01 N H₂SO₄, and the chromatography run was performed at a flow rate of 0.6 mL/min. The column temperature was maintained at 50° C., and detection was by refractive index. Alternately, the amounts of sugar were analyzed using a Biorad Aminex HPX-87H column with a Waters 2410 refractive index detector. The analysis time was about 20 min, the injection volume was 20 μL, the mobile phase was a 0.01 N sulfuric acid, which was filtered through a 0.2 μm filter and degassed, the flow rate was 0.6 mL/min, and the column temperature was maintained at 60° C. External standards of glucose, xylose, and arabinose were run with each sample set.

Size exclusion chromatography was used to separate and identify oligomeric sugars. A Tosoh Biosep G2000PW column 7.5 mm×60 cm was used. Distilled water was used to elute the sugars. A flow rate of 0.6 mL/min was used, and the column was run at room temperature. Six carbon sugar standards included stachyose, raffinose, cellobiose and glucose; five carbon sugar standards included xylohexose, xylopentose, xylotetrose, xylotriose, xylobiose and xylose. Xylo-oligomer standards were purchased (Megazyme). Detection was by refractive index. Either peak area units or relative peak area by percent was used to report the results.

Total soluble sugars were determined by hydrolysis of the centrifuged and filter-clarified samples (above). The clarified sample was diluted 1:1 using 0.8 N H₂SO₄. The resulting solution was autoclaved in a capped vial for 1 h at 121° C. Results are reported without correction for loss of monomer sugar during hydrolysis.

6.1.6. F. Oligomer Preparation from Cob and Enzyme Assays

Oligomers from T. reesei Xyn3 hydrolysis of corncobs were prepared by incubating 8 mg T. reesei Xyn3 per g Glucan+Xylan with 250 g dry weight of dilute ammonia pretreated corncob in a 50 mM pH 5.0 sodium acetate buffer. The reaction proceeded for 72 h at 48° C., with rotary shaking at 180 rpm. The supernatant was centrifuged 9,000×G, then filtered through 0.22 μm Nalgene filters to recover the soluble sugars.

6.1.7. G. Corncob Saccharification Assay

For typical examples herein, corncob saccharification assays were performed in a micro titer plate format in accordance with the following procedures, unless a particular example indicated specific variations. The biomass substrate, e.g., the dilute ammonia pretreated corncob, was diluted in water and pH-adjusted with sulfuric acid to create a pH 5, 7% cellulose slurry that was used without further processing in the assay. Enzyme samples were loaded based on mg total protein per g of cellulose (as determined using conventional compositional analysis methods, supra) in the corncob substrate. The enzymes were diluted in 50 mM sodium acetate, pH 5.0, to obtain the desired loading concentrations. Forty (40) μL of enzyme solution were added to 70 mg of dilute-ammonia pretreated corncob at 7% cellulose per well (equivalent to 4.5% cellulose final per well). The assay plates were then covered with aluminum plate sealers, mixed at room temperature, and incubated at 50° C., 200 rpm, for 3 d. At the end of the incubation period, the saccharification reaction was quenched by the addition to each well of 100 μL of a 100 mM glycine buffer, pH10.0, and the plate was centrifuged for 5 min at 3,000 rpm. Ten (10) μL of the supernatant was added to 200 μL of MilliQ water in a 96-well HPLC plate and the soluble sugars were measured by HPLC.

6.1.8. H. Cellobiose Hydrolysis Assay

Cellobiase activity was determined using the method of Ghose, T. K. Pure and Applied Chemistry, 1987, 59(2), 257-268. Cellobiose units (derived as described in Ghose) are defined as 0.815 divided by the amount of enzyme required to release 0.1 mg glucose under the assay conditions.

6.1.9. I. Chloro-Nitro-Phenyl-Glucoside (CNPG) Hydrolysis Assay

Two hundred (200) μL of a 50 mM sodium acetate buffer, pH 5 was added to individual wells of a microtiter plate. The plate was covered and allowed to equilibrate at 37° C. for 15 min in an Eppendorf Thermomixer. Five (5) μL of enzyme, diluted in 50 mM sodium acetate buffer, pH 5, was also added to individual wells. The plate was covered again, and allowed to equilibrate at 37° C. for 5 min. Twenty (20) μL of 2 mM 2-Chloro-4-nitrophenyl-β-D-Glucopyranoside (CNPG, Rose Scientific Ltd., Edmonton, Calif.) prepared in Millipore water was added to individual wells and the plate was quickly transferred to a spectrophotometer (SpectraMax 250, Molecular Devices). A kinetic read was performed at OD 405 nm for 15 min and the data recorded as Vₘₐₓ. The extinction coefficient for CNP was used to convert Vmax from units of OD/sec to μM CNP/sec. Specific activity (μM CNP/sec/mg Protein) was determined by dividing μM CNP/sec by the mg of enzyme protein used in the assay.

6.1.10. J. Microtiter Plate Saccharification Assay

Purified cellulases and whole cellulase strain cell-free products were introduced into the saccharification assay in an amount based on the total protein (in mg) per g cellulose in the substrate. Purified hemicellulases were loaded based on the xylan content of the substrate. Biomass substrates, including, e.g., dilute acid-pretreated cornstover (PCS), ammonia fiber expanded (AFEX) cornstover, ammonia pretreated corncob, sodium hydroxide (NaOH) pretreated corncob, and ammonia pretreated switchgrass, were mixed at the indicated % solids levels and the pH of the mixtures was adjusted to 5.0. The plates were covered with aluminum plate sealers and placed in incubators, which was preset at 50° C. Incubation took place with shaking, for 2 d. The reactions were terminated by adding 100 μL 100 mM glycine, pH 10 to individual wells. After thorough mixing, the plates were centrifuged and the supernatants were diluted 10 fold into an HPLC plate containing 100 μL 10 mM glycine buffer, pH 10. The concentrations of soluble sugars produced were measured using HPLC as described for the Cellobiose hydrolysis assay (below). The percent glucan conversion is defined as [mg glucose+(mg cellobiose×1.056+mg cellotriose×1.056)]/[mg cellulose in substrate×1.111]; % xylan conversion is defined as [mg xylose+(mg xylobiose×1.06)]/[mg xylan in substrate×1.136].

6.1.11. K. Calcofluor Assay

All chemicals used were of analytical grade. Avicel PH-101 was purchased from FMC BioPolymer (Philadelphia, Pa.). Cellobiose and calcofluor white were purchased from Sigma (St. Louise, Mo.). Phosphoric acid swollen cellulose (PASC) was prepared from Avicel PH-101 using an adapted protocol of Walseth, TAPPI 1971, 35:228 and Wood, Biochem. J. 1971, 121:353-362. In short, Avicel was solubilized in concentrated phosphoric acid then precipitated using cold deionized water. After the cellulose is collected and washed with more water to neutralize the pH, it was diluted to 1% solids in 50 mM sodium acetate pH5. All enzyme dilutions were made into 50 mM sodium acetate buffer, pH5.0. GC220 Cellulase (Danisco US Inc., Genencor) was diluted to 2.5, 5, 10, and 15 mg protein/G PASC, to produce a linear calibration curve. Samples to be tested were diluted to fall within the range of the calibration curve, i.e. to obtain a response of 0.1 to 0.4 fraction product. 150 μL of cold 1% PASC was added to 20 μL of enzyme solution in 96-well microtiter plates. The plate was covered and incubated for 2 h at 50° C., 200 rpm in an Innova incubator/shaker. The reaction was quenched with 100 μL of 50 μg/mL Calcofluor in 100 mM Glycine, pH10. Fluorescence was read on a fluorescence microplate reader (SpectraMax M5 by Molecular Devices) at excitation wavelength Ex=365 nm and emission wavelength Em=435 nm. The result is expressed as the fraction product according to the equation:

FP=1−(Fl sample−Fl buffer w/cellobiose)/(Fl zero enzyme−Fl buffer w/cellobiose),

wherein FP is fraction product, and Fl=fluorescence units

6.1.12. L. Sophorose Hydrolysis Assay

The assay for testing the sophorase activity of the β-glucosidases was performed on microtiter plate scale using sophorose purchased from Sigma Aldrich (S1404). The sophorose was suspended in 50 mM sodium acetate, pH 5.0, to create a stock solution of 5 mg/mL, and it was placed on rotator mixer for 30 min at room temperature. The sophorose (50 μL per well) was dispensed into a flat bottom, non-binding 96 well microtiter plate (corning, 04809009). The dispensed substrate was stored at room temperature for 5 min. In a second flat bottom 96 well microtiter plate (corning, 04809009) the β-glucosidase molecules were serially diluted in 10-fold in 50 mM sodium acetate, pH 5.0. The reaction plate was sealed with aluminum plate seals (E&K scientific) and was incubated at 37° C. and 600 rpm for 30 min (ThermoCycler). At the end of the incubation period, the reactions were serially diluted, 2-fold, across plate in 50 mM sodium acetate, pH 5.0. In a third flat bottom 96 well microtiter plate (Corning, 04809009), 10 μL of diluted enzyme sample or glucose standard were added to 90 μL of ABTS reagent. The kinetics of the reaction was observed at 420 nm, for 5 min, every 15 sec. The glucose concentration was determined using the glucose standard (5 mg/mL).

### 6.2 Example 2

### Construction of the Integrated Expression Strain of T. Reesei

An integrated expression strain of T. reesei was constructed that co-expressed five genes: T. reesei β-glucosidase gene bgl1, T. reesei endoxylanase gene xyn3, F. verticillioides xylosidase gene fv3A, F. verticillioides β-xylosidase gene fv43D, and F. verticillioides α-arabinofuranosidase gene fv51A.

The construction of the expression cassettes for these different genes and the transformation of T. reesei are described below.

6.2.1. A. Construction of the β-Glucosidase Expression Vector

The N-terminal portion of the native T. reesei β-glucosidase gene bgl1 was codon optimized by DNA 2.0 (Menlo Park, USA). This synthesized portion comprised of the first 447 bases of the coding region. This fragment was PCR amplified using primers SK943 and SK941. The remaining region of the native bgl1 gene was PCR amplified from a genomic DNA sample extracted from T. reesei strain RL-P37 (Sheir-Neiss, G et al. Appl. Microbiol. Biotechnol. 1984, 20:46-53), using primer SK940 and SK942. These two PCR fragments of the bgl1 gene were fused together in a fusion PCR reaction, using primers SK943 and SK942:

Forward Primer SK943:
(SEQ ID NO: 118)
(5′-CACCATGAGATATAGAACAGCTGCCGCT-3′)
Reverse Primer SK941:
(SEQ ID NO: 119)
(5′-CGACCGCCCTGCGGAGTCTTGCCCAGTGGTCCCGCGACAG-3′)
Forward Primer (SK940):
(SEQ ID NO: 120)
(5′-CTGTCGCGGGACCACTGGGCAAGACTCCGCAGGGCGGTCG-3′)
Reverse Primer (SK942):
(SEQ ID NO: 121)
(5′-CCTACGCTACCGACAGAGTG-3′)

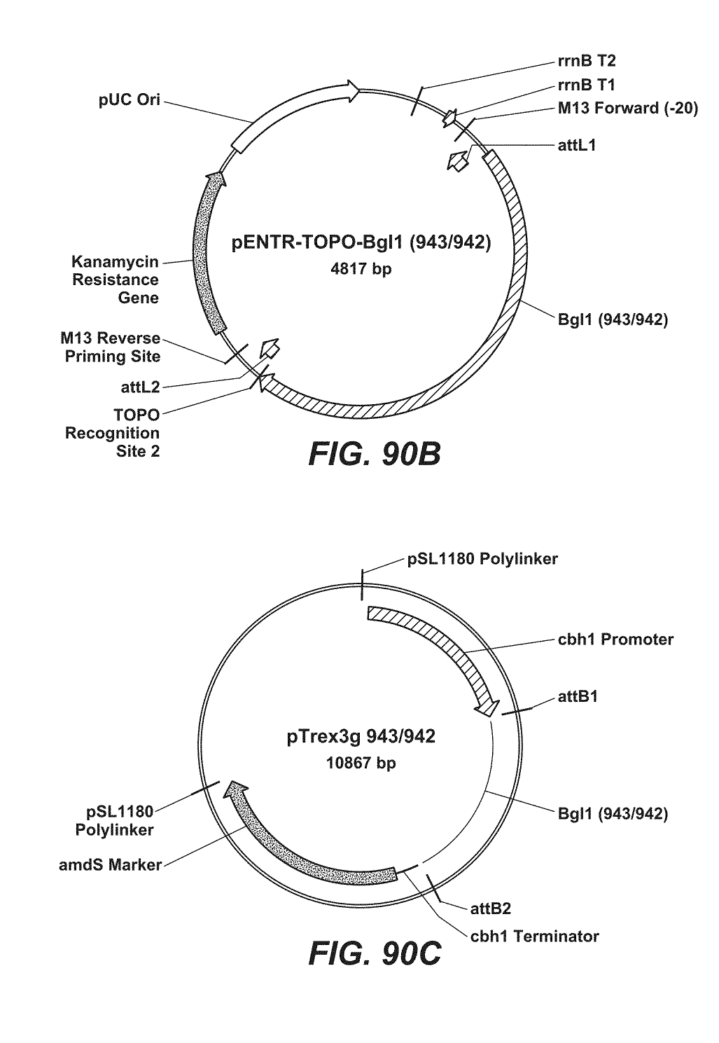

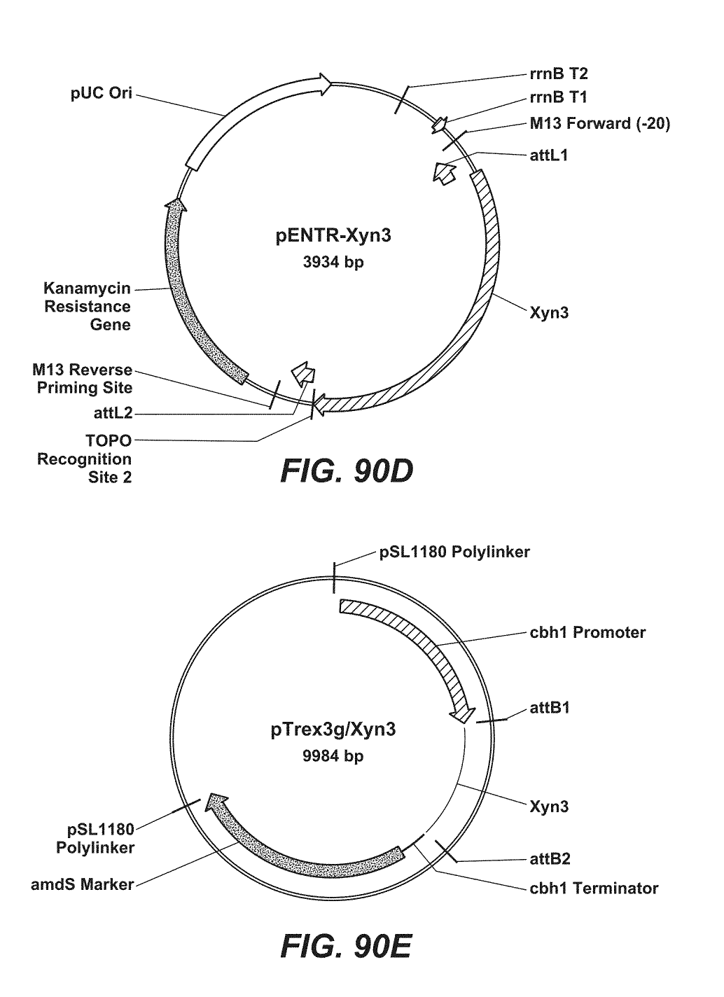

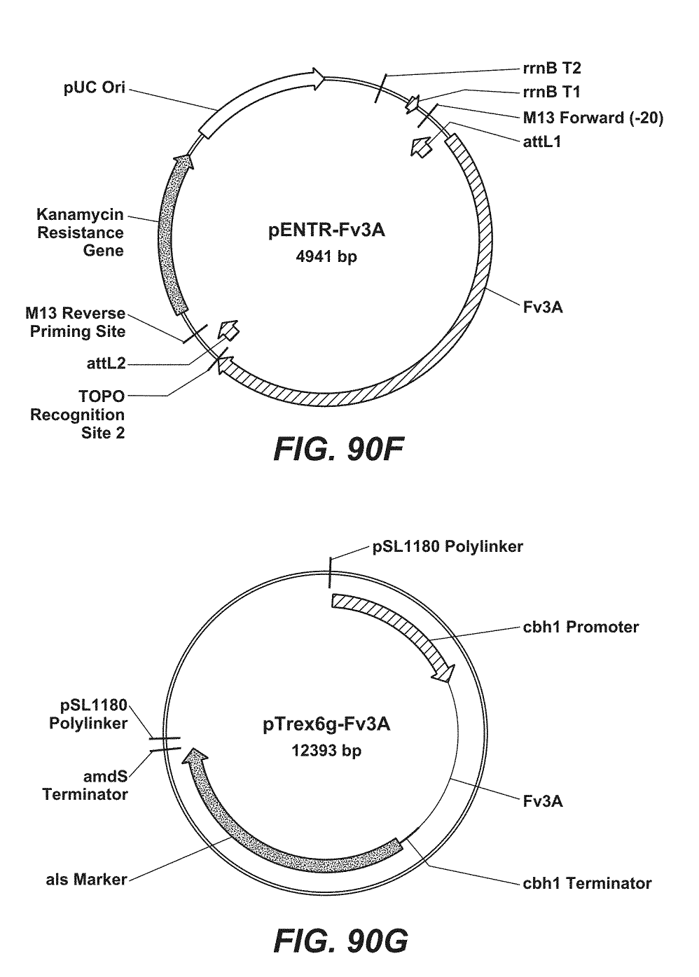

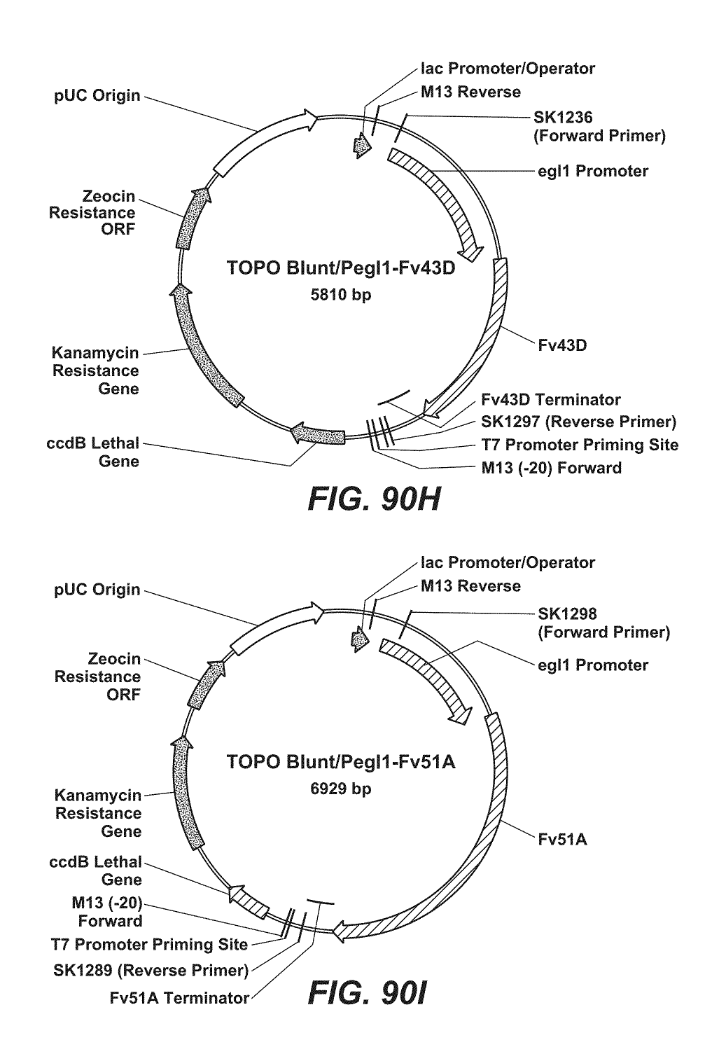

The resulting fusion PCR fragments were cloned into the Gateway® Entry vector pENTR™/D-TOPO®, and transformed into E. coli One Shot® TOP10 Chemically Competent cells (Invitrogen) resulting in the intermediate vector, pENTR-TOPO-Bgl1(943/942) (FIG. 90B). The nucleotide sequence of the inserted DNA was determined.

The pENTR-943/942 vector with the correct bgl1 sequence was recombined with pTrex3g using a LR clonase® reaction protocol outlined by Invitrogen. The LR clonase reaction mixture was transformed into E. coli One Shot® TOP10 Chemically Competent cells (Invitrogen), resulting in the final expression vector, pTrex3g 943/942 (FIG. 90C). The vector also contains the Aspergillus nidulans amdS gene, encoding acetamidase, as a selectable marker for transformation of T. reesei. The expression cassette was amplified by PCR with primers SK745 and SK771 to generate product for transformation of T. reesei.

| Forward Primer SK771: | |
|---|---|
| (5′-GTCTAGACTGGAAACGCAAC-3′) | (SEQ ID NO: 122) |
| Reverse Primer SK745: | |
| (5′-GAGTTGTGAAGTCGGTAATCC-3′) | (SEQ ID NO: 123) |

6.2.2 B. Construction of the Endoxylanase Expression Cassette

The native T. reesei endoxylanase gene xyn3 was PCR amplified from a genomic DNA sample extracted from T. reesei, using primers xyn3F-2 and xyn3R-2.

Forward Primer xyn3F-2:
(SEQ ID NO: 24)
(5′-CACCATGAAAGCAAACGTCATCTTGTGCCTCCTGG-3′)
Reverse Primer xyn3R-2:
(SEQ ID NO: 125)
(5′-CTATTGTAAGATGCCAACAATGCTGTTATATGCCGGCTTGGGG-3′)

The resulting PCR fragments were cloned into the Gateway® Entry vector pENTR™/D-TOPO®, and transformed into E. coli One Shot® TOP10 Chemically Competent cells, see FIG. 90D). The nucleotide sequence of the inserted DNA was determined. The pENTR/Xyn3 vector with the correct xyn3 sequence was recombined with pTrex3g using a LR clonase® reaction protocol outlined by Invitrogen. The LR clonase reaction mixture was transformed into E. coli One Shot® TOP10 Chemically Competent cells (Invitrogen), resulting in the final expression vector, pTrex3g/Xyn3 (FIG. 90E). The vector also contains the Aspergillus nidulans amdS gene, encoding acetamidase, as a selectable marker for transformation of T. reesei. The expression cassette was amplified by PCR with primers SK745 and SK822 to generate product for transformation of T. reesei.

Forward Primer SK745:
(SEQ ID NO: 126)
(5′-GAGTTGTGAAGTCGGTAATCC-3′)
Reverse Primer SK822:
(SEQ ID NO: 127)
(5′-CACGAAGAGCGGCGATTC-3′)

6.2.3. C. Construction of the β-Xylosidase Fv3A Expression Vector

The F. verticillioides β-xylosidase fv3A gene was amplified from a F. verticillioides genomic DNA sample using the primers MH124 and MH125.

Forward Primer MH124:
(SEQ ID NO: 128)
(5′-CAC CCA TGC TGC TCA ATC TTC AG-3′)
Reverse Primer MH125:
(SEQ ID NO: 129)
(5′-TTA CGC AGA CTT GGG GTC TTG AG-3′)

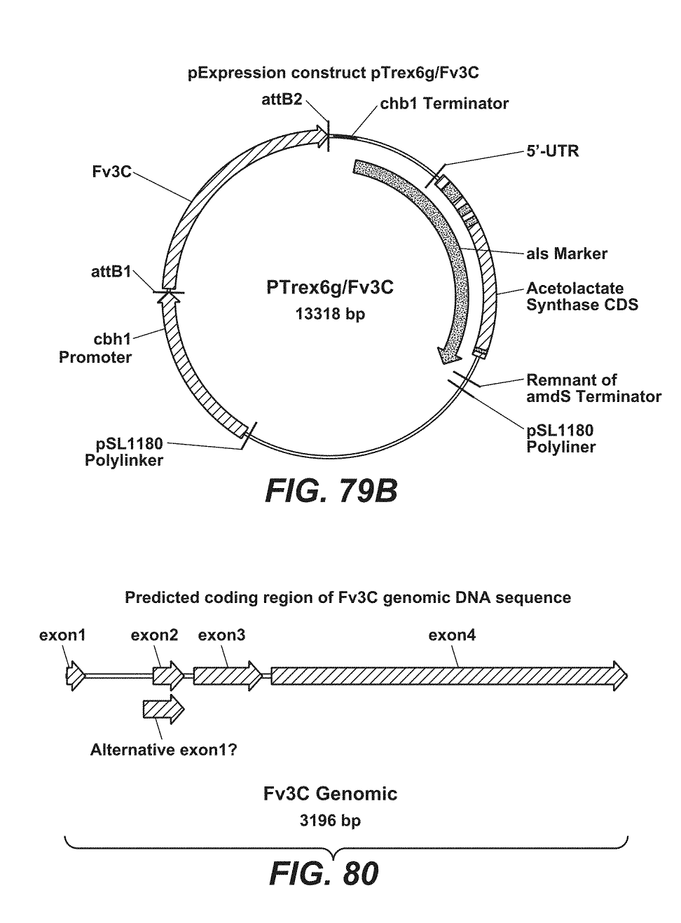

The PCR fragments were cloned into the Gateway® Entry vector pENTR™/D-TOPO®, and transformed into E. coli One Shot® TOP10 Chemically Competent cells (Invitrogen) resulting in the intermediate vector, pENTR-Fv3A (FIG. 90F). The nucleotide sequence of the inserted DNA was determined. The pENTR-Fv3A vector with the correct fv3A sequence was recombined with pTrex6g (FIG. 79A) using a LR clonase® reaction protocol outlined by Invitrogen. The LR clonase reaction mixture was transformed into E. coli One Shot® TOP10 Chemically Competent cells (Invitrogen), resulting in the final expression vector, pTrex6g/Fv3A (FIG. 90G). The vector also contains a chlorimuron ethyl resistant mutant of the native T. reesei acetolactate synthase (als) gene, designated alsR, which is used together with its native promoter and terminator as a selectable marker for transformation of T. reesei (WO2008/039370 A1). The expression cassette was PCR amplified with primers SK1334, SK1335 and SK1299 to generate product for transformation of T. reesei.

Forward Primer SK1334:
(SEQ ID NO: 130)
(5′-GCTTGAGTGTATCGTGTAAG-3′)
Forward Primer SK1335:
(SEQ ID NO: 131)
(5′-GCAACGGCAAAGCCCCACTTC-3′)
Reverse Primer SK1299:
(SEQ ID NO: 132)
(5′-GTAGCGGCCGCCTCATCTCATCTCATCCATCC-3′)

6.2.4. D. Construction of the β-Xylosidase Fv43D Expression Cassette

For the construction of the F. verticillioides β-xylosidase Fv43D expression cassette, the fv43D gene product was amplified from a F. verticillioides genomic DNA sample using the primers SK1322 and SK1297. A region of the promoter of the endoglucanase gene egl1 was amplified by PCR from a T. reesei genomic DNA sample extracted from strain RL-P37, using the primers SK1236 and SK1321. These two PCR amplified DNA fragments were subsequently fused together in a fusion PCR reaction using the primers SK1236 and SK1297. The resulting fusion PCR fragment was cloned into pCR-Blunt II-TOPO vector (Invitrogen) to give the plasmid TOPO Blunt/Pegl1-Fv43D (FIG. 90H) and E. coli One Shot® TOP10 Chemically Competent cells (Invitrogen) were transformed using this plasmid. Plasmid DNA was extracted from several E. coli clones and confirmed by restriction digest.

Forward Primer SK1322:
(SEQ ID NO: 133)
(5′-CACCATGCAGCTCAAGTTTCTGTC-3′)
Reverse Primer SK1297:
(SEQ ID NO: 134)
(5′-GGTTACTAGTCAACTGCCCGTTCTGTAGCGAG-3′)
Forward Primer SK1236:
(SEQ ID NO: 135)
(5′-CATGCGATCGCGACGTTTTGGTCAGGTCG-3′)
Reverse Primer SK1321:
(SEQ ID NO: 136)
(5′-GACAGAAACTTGAGCTGCATGGTGTGGGACAACAAGAAGG-3′)

The expression cassette was PCR amplified from TOPO Blunt/Pegl1-Fv43D with primers SK1236 and SK1297 to generate product for transformation of T. reesei.

6.2.5. E. Construction of the α-Arabinofuranosidase Expression Cassette

For the construction of the F. verticillioides α-arabinofuranosidase gene fv51A expression cassette, the fv51A gene product was amplified from F. verticillioides genomic DNA sample using the primers SK1159 and SK1289. A region of the promoter of the endoglucanase gene egl1 was amplified by PCR from a T. reesei genomic DNA sample extracted from strain RL-P37, using the primers SK1236 and SK1262. These two PCR amplified DNA fragments were subsequently fused together in a fusion PCR reaction using the primers SK1236 and SK1289. The resulting fusion PCR fragment was cloned into pCR-Blunt II-TOPO vector (Invitrogen) to give the plasmid TOPO Blunt/Pegl1-Fv51A (FIG. 90I) and E. coli One Shot® TOP10 Chemically Competent cells (Invitrogen) were transformed using this plasmid.

Forward Primer SK1159:
(SEQ ID NO: 137)
(5′-CACCATGGTTCGCTTCAGTTCAATCCTAG-3′)
Reverse Primer SK1289:
(SEQ ID NO: 138)
(5′-GTGGCTAGAAGATATCCAACAC-3′)
Forward Primer SK1236:
(SEQ ID NO: 139)
(5′-CATGCGATCGCGACGTTTTGGTCAGGTCG-3′)
Reverse Primer SK1262:
(SEQ ID NO: 140)
(5′-GAACTGAAGCGAACCATGGTGTGGGACAACAAGAAGGAC-3′)

The expression cassette was PCR amplified with primers SK1298 and SK1289 to generate product for transformation of T. reesei.

Forward Primer SK1298:
(SEQ ID NO: 141)
(5′-GTAGTTATGCGCATGCTAGAC-3′)
Reverse Primer SK1289:
(SEQ ID NO: 142)
(5′-GTGGCTAGAAGATATCCAACAC-3′)

6.2.6. F. Co-Transformation of T. Reesei Expression Cassettes for β-Glucosidase and Endoxylanase

A T. reesei mutant strain, derived from RL-P37 (Sheir-Neiss, G et al. Appl. Microbiol. Biotechnol. 1984, 20:46-53.) and selected for high cellulase production was co-transformed with the β-glucosidase expression cassette (cbh1 promoter, T. reesei β-glucosidase1 gene, cbh1 terminator, and amdS marker), and the endoxylanase expression cassette (cbh1 promoter, T. reesei xyn3, and cbh1 terminator) using PEG-mediated transformation (Penttila, M et al. Gene 1987, 61(2):155-64). Numerous transformants were isolated and examined for β-glucosidase and endoxylanase production. One transformant called T. reesei strain #229 was used for transformation with the other expression cassettes.

6.2.7. G. Co-Transformation of T. Reesei Strain #229 with Expression Cassettes for Two β-Xylosidases and an α-Arabinofuranosidase

T. reesei strain #229 was co-transformed with the β-xylosidase fv3A expression cassette (cbh1 promoter, fv3A gene, cbh1 terminator, and alsR marker), the β-xylosidase fv43D expression cassette (egl1 promoter, fv43D gene, native fv43D terminator), and the fv51A α-arabinofuranosidase expression cassette (egl1 promoter, fv51A gene, fv51A native terminator) using electroporation (see e.g. WO 08153712). Transformants were selected on Vogels agar plates containing chlorimuron ethyl (80 ppm). Vogels agar was prepared as follows, per liter.

| | | |
|---|---|---|
| 50 × Vogels Stock Solution (recipe below) | 20 | mL |
| BBL Agar | 20 | g |
| With deionized H₂O bring to | 980 | mL |
| post-sterile addition: | | |
| 50% Glucose | 20 | mL |
| 50 × Vogels Stock Solution, per liter: | | |
| In 750 mL deionized H2O, dissolve successively: | | |
| Na₃Citrate*2H₂O | 125 | g |
| KH₂PO₄ (Anhydrous) | 250 | g |
| NH₄NO₃ (Anhydrous) | 100 | g |
| MgSO₄*7H₂O | 10 | g |
| CaCl₂*2H₂O | 5 | g |
| Vogels Trace Element Solution (recipe below) | 5 | mL |
| d-Biotin | 0.1 | g |
| With deionized H₂O, | bring to 1 | L |
| Vogels Trace Element Solution: | | |
| Citric Acid | 50 | g |
| ZnSO₄•*7H₂O | 50 | g |
| Fe(NH₄)2SO₄•*6H₂O | 10 | g |
| CuSO₄•5H₂O | 2.5 | g |
| MnSO₄•4H₂O | 0.5 | g |
| H₃BO₃ | 0.5 | g |
| Na₂MoO₄•2H₂O | 0.5 | g |

Numerous transformants were isolated and examined for β-xylosidase and L-α-arabinofuranosidase production. Transformants were also screened for biomass conversion performance according to the cob saccharification assay described in Example 1 (supra). Examples of T. reesei integrated expression strains described herein are H3A, 39A, A10A, 11A, and G9A, which express all of the genes for T. reesei Bgl1, T. reesei Xyn3, Fv3A, Fv51A, and Fv43D, at different ratios. Other integrated T. reesei strains include those wherein most of the genes for T. reesei Bgl1, T. reesei Xyn3, Fv3A, Fv51A, and Fv43D, were expressed at different ratios. For example, one lacked overexpressed T. reesei Xyn3; another lacked Fv51A, as determined by Western Blot; two others lacked Fv3A, one lacked overexpressed Bgl1 (e.g. strain H3A-5).

6.2.8. H. Composition of T. Reesei Integrated Strain H3A

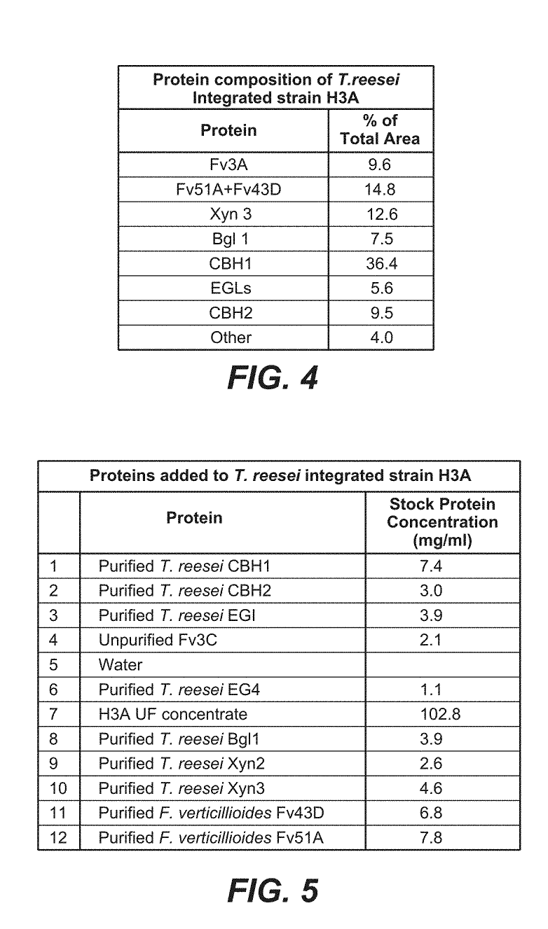

Fermentation of the T. reesei integrated strain H3A yields the following proteins T. reesei Xyn3, T. reesei Bgl 1, Fv3A, Fv51A, and Fv43D, at ratios determined as described in Example 2, I, below and shown in FIG. 4 herein.

6.2.9. I. Protein Analysis by HPLC

Liquid chromatography (LC) and mass spectroscopy (MS) were performed to separate, identify and quantify the enzymes contained in fermentation broths. Enzyme samples were first treated with a recombinantly expressed endoH glycosidase from S. plicatus (e.g., NEB P0702L). EndoH was used at a ratio of 0.01-0.03 μg endoH protein per pg sample total protein and incubated for 3 h at 37° C., pH 4.5-6.0 to enzymatically remove N-linked gycosylation prior to HPLC analysis. Approximately 50 μg of protein was then injected for hydrophobic interaction chromatography using an Agilent 1100 HPLC system with an HIC-phenyl column and a high-to-low salt gradient over 35 min. The gradient was achieved using high salt buffer A: 4 M ammonium sulphate containing 20 mM potassium phosphate pH 6.75 and low salt buffer B: 20 mM potassium phosphate pH 6.75. Peaks were detected with UV light at 222 nm and fractions were collected and identified by mass spectroscopy. Protein concentrations are reported as percent of the total integrated chromatogram area.

6.2.10. J. Effect of Addition of Purified Proteins to the Fermentation Broth of T. Reesei Integrated Strain H3A on Saccharification of Dilute Ammonia Pretreated Corncob

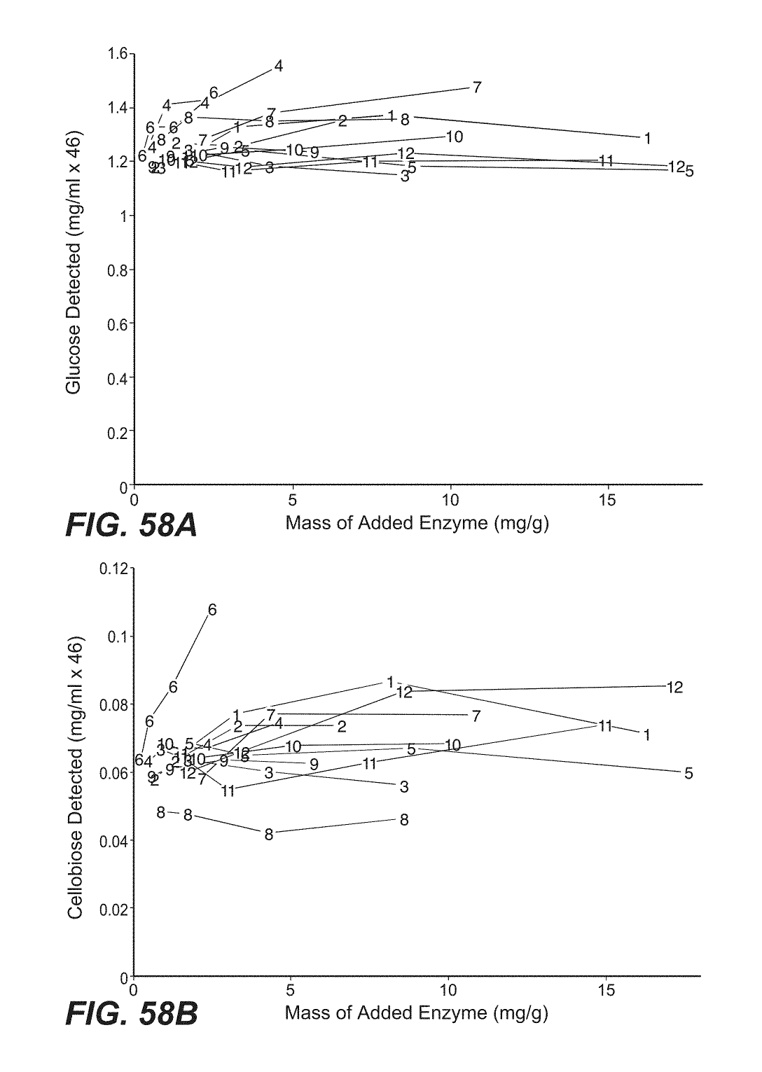

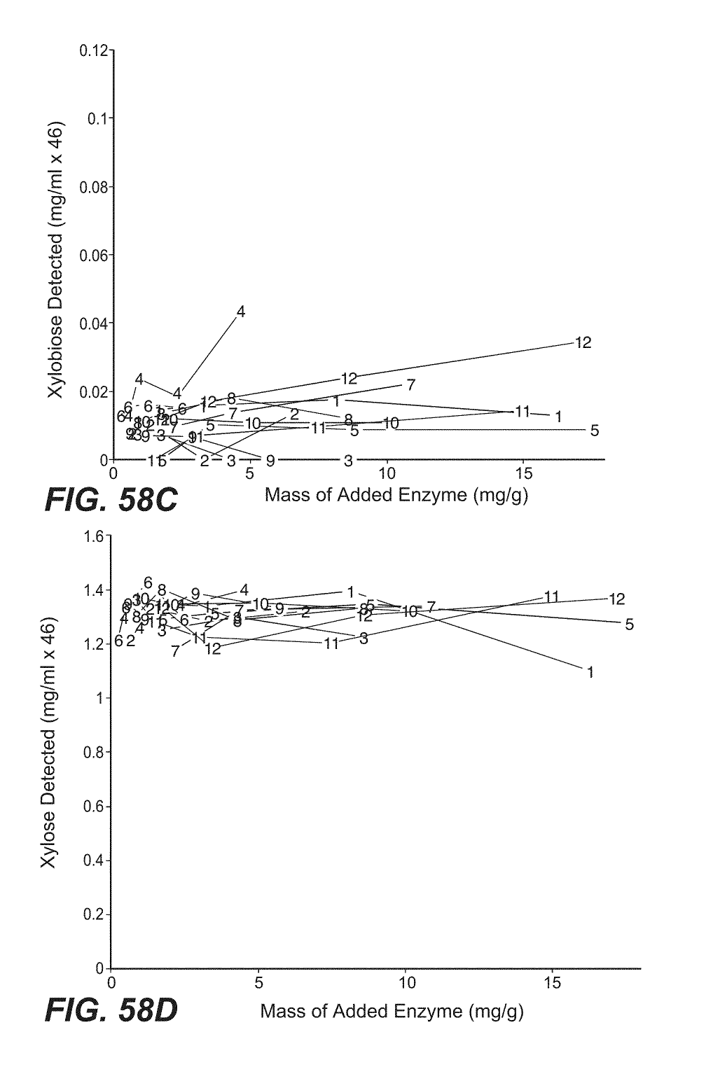

Purified proteins (and one unpurified protein) were serially diluted from stock solutions and added to a fermentation broth of T. reesei integrated strain H3A to determine their benefit to saccharification of pretreated biomass. Dilute ammonia pretreated corncob was loaded into microtiter plate (MTP) wells at 20% solids (w/w) (-5 mg of cellulose per well), pH 5. H3A protein (in the form of fermentation broth) was added to each well at 20 mg protein/g cellulose. Volumes of 10, 5, 2, and 1 μL of each of the diluted proteins (FIG. 5) were added into individual wells, and water was added such that the liquid addition to each well was a total of 10 μL. Reference wells included additions of either 10 μL water or dilutions of additional H3A fermentation broth. The MTP were sealed with foil and incubated at 50° C. with 200 RPM shaking in an Innova incubator shaker for three days. The samples were quenched with 100 μL of 100 mM glycine pH 10. The quenched samples were covered with a plastic seal and centrifuged 3000 RPM for 5 min at 4° C. An aliquot (5 μL) of the quenched reactions was diluted with 100 μL of water and the concentration of glucose produced in the reactions was determined using HPLC. The glucose data was plotted as a function of the protein concentration added to the 20 mg/g of H3A (the concentrations of the protein additions were variable due to different starting concentrations and additions by volume). Results are shown in FIGS. 58A-58D.

### 6.3 Example 3

### Construction of T. Reesei Strains

6.3.1 A. Construction of and Screening for T. Reesei strain H3A/EG4 #27

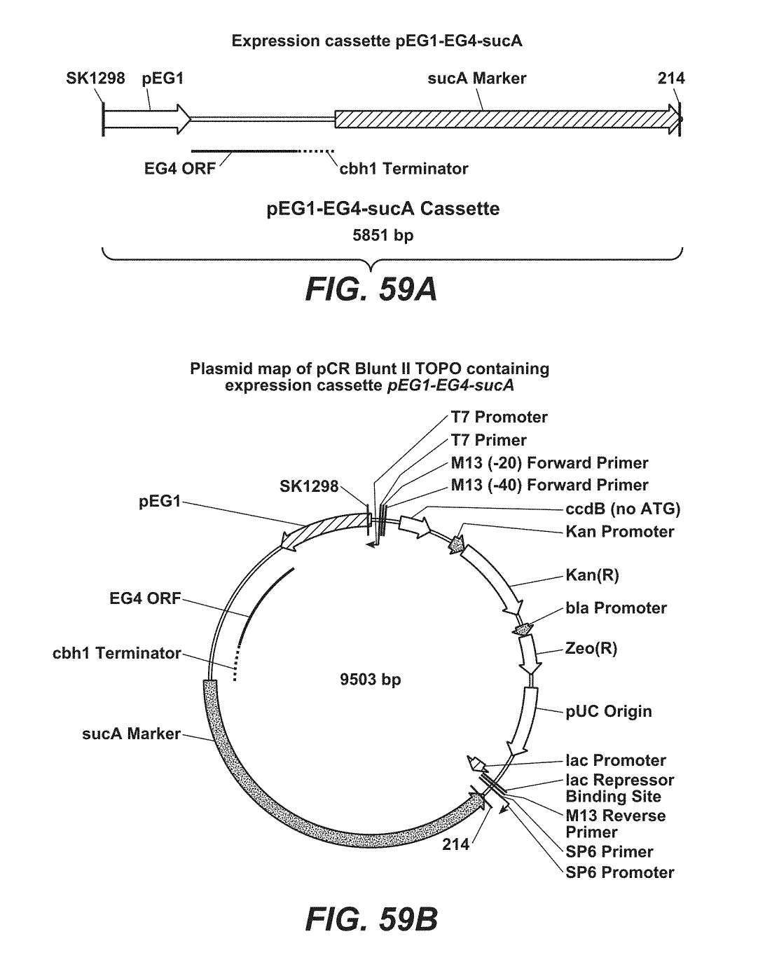

An expression cassette containing the T. reesei egl1 (also termed “Cel 7B”) promoter, T. reesei eg4 (also termed “TrEG4”, or “Cel 61A”) open reading frame, and cbh1 (Cel 7A) terminator sequence (FIG. 59A) from T. reesei, and sucA selectable marker (see, Boddy et al., Curr. Genet. 1993, 24:60-66) from A. niger was cloned into pCR Blunt II TOPO (Invitrogen) (FIG. 59B).

The expression cassette Pegl1-eg4-sucA was amplified by PCR using the following primers:

SK1298:
(SEQ ID NO: 143)
5′-GTAGTTATGCGCATGCTAGAC-3′
214:
(SEQ ID NO: 144)
5′-CCGGCTCAGTATCAACCACTAAGCACAT-3′

Pfu Ultra II (Stratagene) was used as the polymerase for the PCR reaction. The products of the PCR reaction were purified with the QIAquick PCR purification kit (Qiagen) as per the manufacturer's protocol. The products of the PCR reaction were then concentrated using a speed vac to 1-3 μg/μL. The T. reesei host strain to be transformed (H3A) was grown to full sporulation on potato dextrose agar plates for 5 d at 28° C. Spores from 2 plates were harvested with MilliQ water and filtered through a 40 μM cell strainer (BD Falcon). Spores were transferred to a 50 mL conical tube and washed 3 times by repeated centrifugation with 50 mL water. A final wash with 1.1 M sorbitol solution was carried out. The spores were resuspended in a small volume (less than 2 times the pellet volume) using 1.1 M sorbitol solution. The spore suspension was then kept on ice. Spore suspension (60 μl) was mixed with 10-20 μg of DNA, and transferred into the electroporation cuvette (E-shot, 0.1 cm standard electroporation cuvette from Invitrogen). The spores were electroporated using the Biorad Gene Pulser Xcell with settings of 16 kV/cm, 25 μF, 400Ω. After electroporation, 1 mL of 1.1.M sorbitol solution was added to the spore suspension. The spore suspension was plated on Vogel's agar (see example 2G), containing 2% sucrose as the carbon source. The transformation plates were incubated at 30° C. for 5-7 d. The initial transformants were restreaked onto secondary Vogel's agar plates with sucrose and grown at 30° C. for an additional 5-7 d. Single colonies growing on secondary selection plates were then grown in wells of microtiter plates using the method described in WO/2009/114380. The supernatants were analyzed on SDS-PAGE to check for expression levels prior to saccharification performance screening.

A total of 94 transformants overexpressed EG4 in strain H3A. Two H3A control strains were grown in microtiter plates along with the H3A/EG4 strains. Performance screening for T. reesei strains expressing EG4 protein was performed using ammonia pretreated corncob. The dilute ammonia pretreated corncob was suspended in water and adjusted to pH 5.0 with sulfuric acid to achieve 7% cellulose. The slurry was dispensed into a flat bottom 96 well microtiter plate (Nunc, 269787) and centrifuged at 3,000 rpm for 5 min.

Corncob saccharification reactions were initiated by adding 20 μL of H3A or H3A/EG4 strain culture broth per well of substrate. The corncob saccharification reactions were sealed with aluminum (E&K scientific) and mixed for 5 min at 650 rpm, 24° C. The plate was then placed in an Innova incubator at 50° C. and 200 rpm for 72 h. At the end of 72-h saccharification, the reactions were quenched by adding 100 μL of 100 mM glycine, pH 10.0. The plate was then mixed thoroughly and centrifuged at 3000 rpm for 5 min. Supernatant (10 μL) was added to 200 μL of water in an HPLC 96-well microtiter plate (Agilent, 5042-1385). Glucose, xylose, cellobiose and xylobiose concentrations were measured by HPLC using an Aminex HPX-87P column (300 mm×7.8 mm, 125-0098) pre-fitted with guard column.

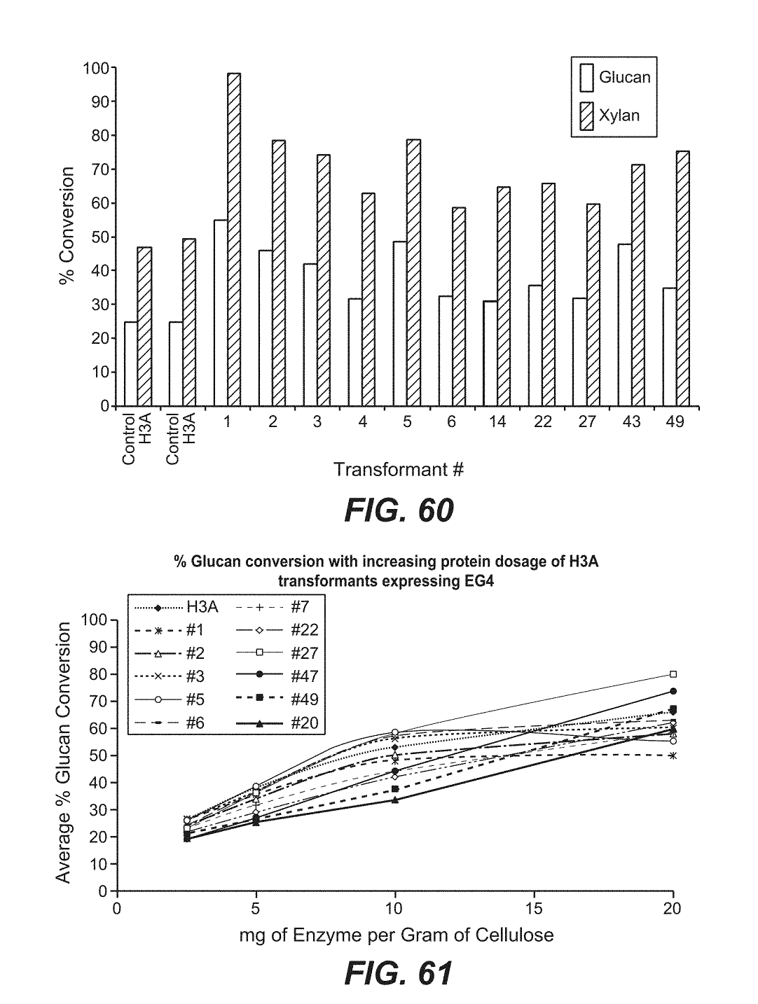

The screening on corncob identified the following H3A/EG4 strains as having improved glucan and xylan conversion compared to the H3A control strains: 1, 2, 3, 4, 5, 6, 14, 22, 27, 43, and 49 (FIG. 60).

Select H3A/EG4 strains were re-grown in shake flasks. A total of 30 mL of protein culture filtrate was collected per shake flask per strain. The culture filtrates were concentrated 10-fold using 10 kDa membrane centrifugal concentrators (Sartorious, VS2001) and the total protein concentration was determined by BCA as described in Example 10. A corncob saccharification reaction was performed using 2.5, 5, 10, or 20 mg protein from H3A/EG4 strain samples per g of cellulose per well of corncob substrate. An H3A strain produced at 14 L fermentation scale and a previously identified low performance sample (H3A/EG4 strain #20) produced at shake flask scale were included as controls. The saccharification reactions were carried out as described in Example 4 (below). Increased glucan conversion with increased protein dose was observed with culture supernatant from all of the EG4 expressing strains (FIG. 61). T. reesei integrated strain H3A/EG4 #27 was used in additional saccharification reactions, and the strain was purified by streaking a single colony onto a potato dextrose plate from which a single colony was isolated.

### 6.4. Example 4

### Range of T. Reesei EG4 Concentrations for Improved Saccharification of Dilute Ammonia Pretreated Corncob

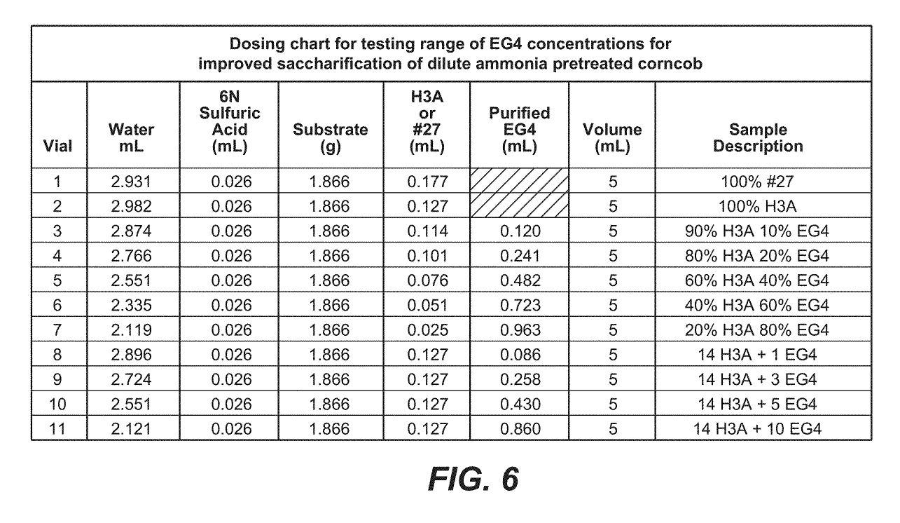

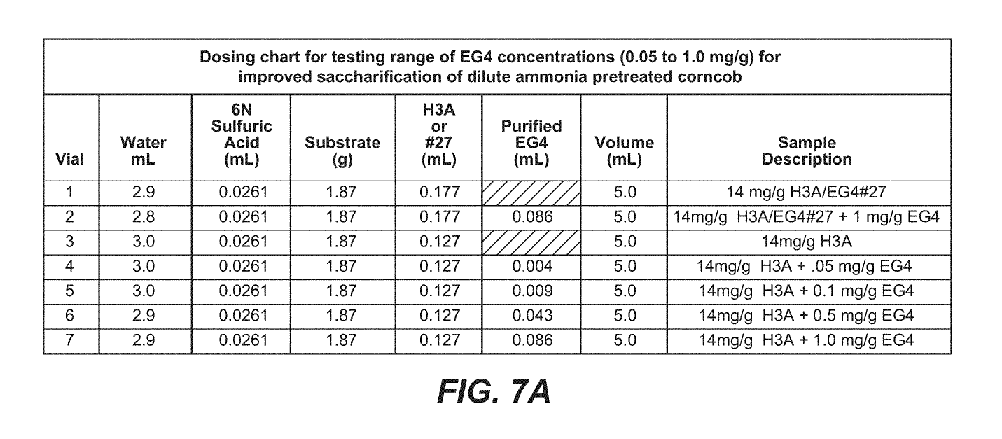

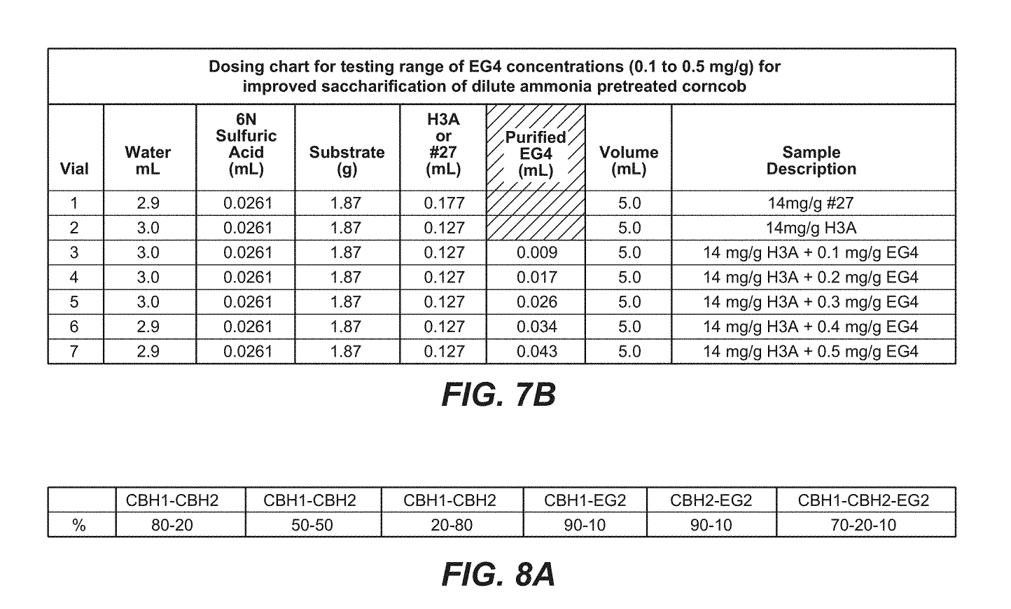

To determine preferred dosing, hydrolysis of dilute ammonia pretreated corncob (25% solids, 8.7% cellulose, 7.3% xylan) was conducted at pH 5.3 using fermentation broth from either T. reesei integrated strain H3A/EG4 #27 or H3A with purified EG4 added to the reaction mix. The total loading of T. reesei integrated strain H3A/EG4 #27 or H3A was 14 mg protein per gram of glucan (G) and xylan (X). The reaction mix (total mass 5 g) was loaded into 20 mL scintillation vials in a total reaction volume of 5 mL according to the dosing charts in FIGS. 6, 7A, and 7B.

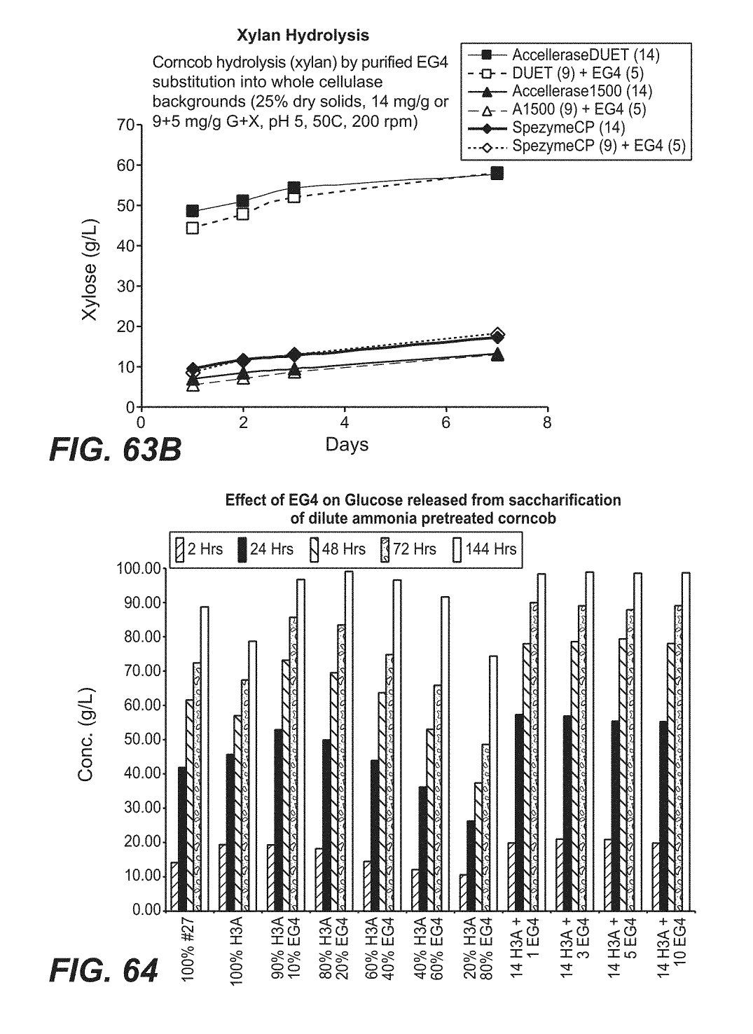

The set up for Experiment 1 is shown in FIG. 6. MilliQ Water and 6 N Sulfuric acid were mixed in a conical tube and added to the respective vials and the vials were swirled to mix the contents. Enzymes samples were added to the vials and the vials incubated for 6 d at 50° C. At varying time points, 100 μL of sample from the vials was diluted with 900 μL 5mM sulfuric acid, vortexed, centrifuged and the supernatant was used to measure the concentrations of soluble sugars produced using HPLC. The results of glucan conversion are shown in FIG. 64 and xylan conversion in FIG. 65.

The set up for Experiment 2 is shown in FIG. 7A. To further determine the preferred EG4 concentration, saccharification of dilute ammonia corncob (25% solids, 8.7% cellulose, 7.3% xylan) was conducted at pH 5.3 using fermentation broth from either T. reesei integrated strain H3A/EG4 #27 or H3A with purified EG4 added (ranging from 0.05 to 1.0 mg protein/g G+X) to the reaction mix. The total loading of T. reesei integrated strain H3A/EG4 #27 or H3A was 14 mg protein/g glucan+xylan.

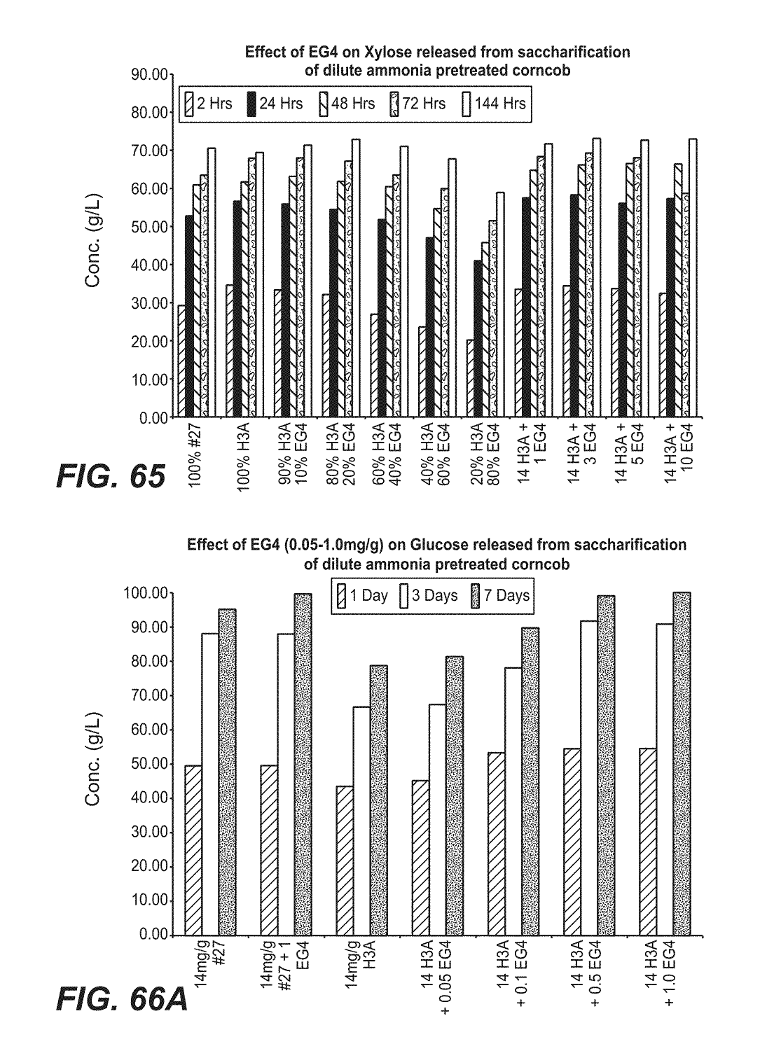

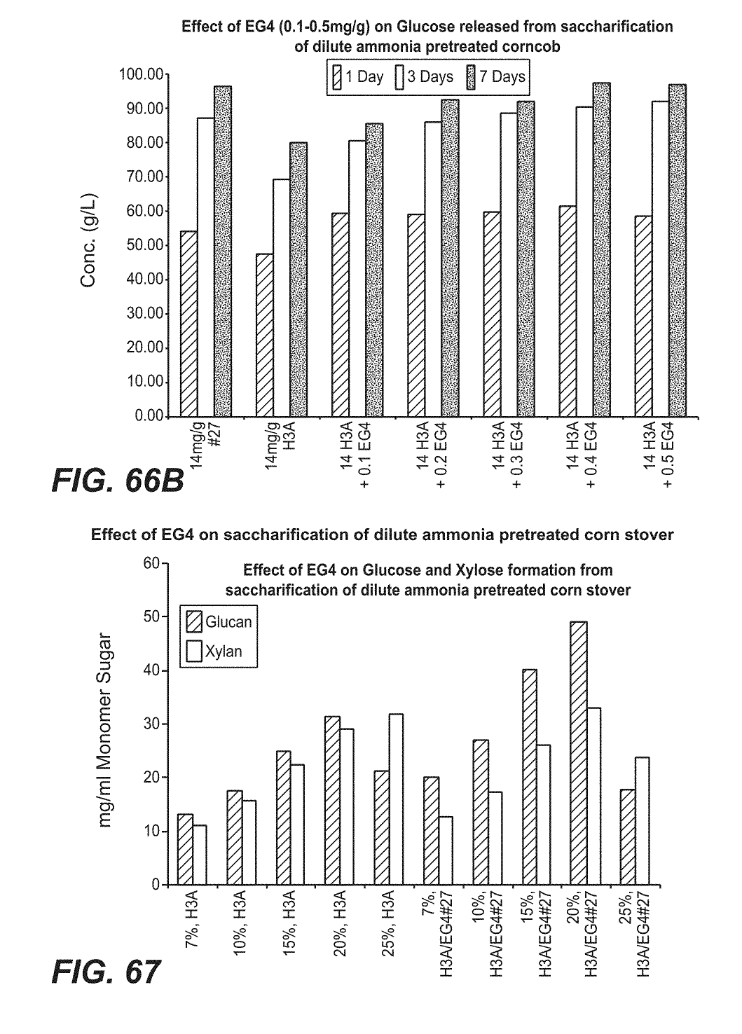

The experimental results are shown in FIG. 66A.

The set up for Experiment 3 is shown in FIG. 7B. To pinpoint the preferred concentration range of T. reesei Eg4 yet further, dilute ammonia corncob (25% solids, 8.7% cellulose, and 7.3% xylan) was hydrolyzed at pH 5.3 using T. reesei integrated strain H3A/EG4 #27 or H3A with purified EG4 added at concentrations ranging from 0.1-0.5 mg protein/g G+X. The total loading of T. reesei integrated strain H3A/EG4 #27 or H3A was 14 mg protein per g of glucan and xylan.

Results are shown in FIG. 66B.

### 6.5 Example 5

### Effect of T. Reesei Eg4 on Saccharification of Dilute Ammonia Pretreated Corn Stover at Different Loadings

Dilute ammonia pre-treated corn stover was incubated with fermentation broth from T. reesei integrated strain H3A or H3A/EG4 #27 (14 mg protein/g glucan and xylan) at 7, 10, 15, 20 and 25% solids (%S) for three days at 50° C., pH 5.3 (5 g total wet biomass in 20 mL vials). The reactions were carried out as described in Example 4 above. Glucose and xylose were analyzed by HPLC. Results are shown in FIG. 67. All samples up to 20% solids were visibly liquefied at day 1.

### 6.6 Example 6

### Effect of Overexpression of T. Reesei EG4 on Hydrolysis of Dilute Ammonia Pretreated Corncob

The effect of overexpression of T. reesei Eg4 in strain H3A on saccharification of dilute ammonia pretreated corncob was tested using fermentation broths from strains H3A/EG4 #27 and H3A. Corncob saccharification at 3 g scale was performed in 20 mL glass vials as follows. Enzyme preparation, 1 N sulfuric acid and 50 mM pH 5.0 sodium acetate buffer (with 0.01% sodium azide and 5 mM MnCl₂) were added to give a final slurry of 3 g total reaction, 22% dry solids, pH 5.0 with enzyme loadings varying between 1.7 and 21.0 mg total protein per gram Glucan+Xylan. All saccharification vials were incubated at 48° C. with 180 rpm rotation. After 72 h, 12 mL of filtered MilliQ water was added to each vial to dilute the entire saccharification reaction 5-fold. The samples were centrifuged at 14,000×g for 5 min, then filtered through a 0.22 μm nylon filter (Spin-X centrifuge tube filter, Corning Incorporated, Corning, N.Y.) and further diluted 4-fold with filtered MilliQ water to create a final 20× dilution. 20 μL injections were analyzed by HPLC to measure the sugars released.

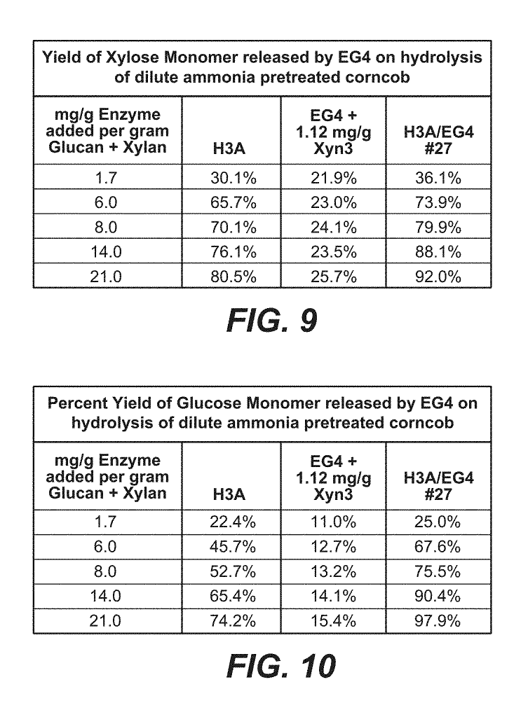

Overexpression or addition of T. reesei Eg4 led to enhanced xylose and glucose monomer release as compared to H3A alone (FIGS. 9 and 10). Addition of H3A/EG4 #27 at different doses led to an increased yield of xylose as compared to strain H3A, or compared to Eg4+ a constant 1.12 mg Xyn3 per g Glucan+Xylan (FIG. 9).

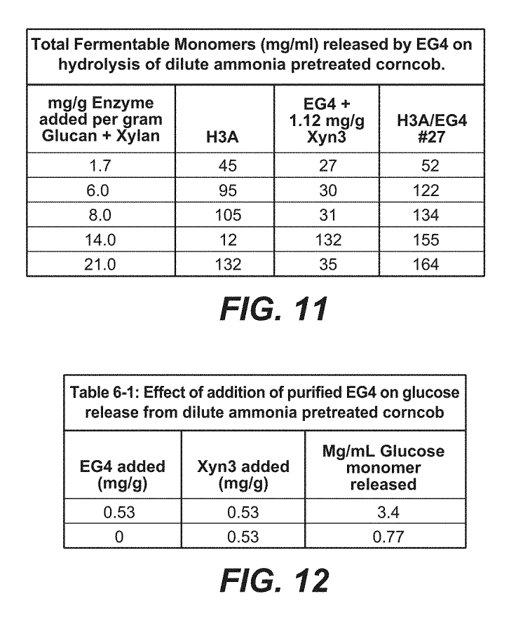

Addition of H3A/EG4 #27 at different doses led to an increased yield of glucose compared to strain H3A or compared to Eg4+a constant 1.12 mg Xyn3 per g Glucan+Xylan (FIG. 10). The effect of T. reesei Eg4 on total fermentable monomer (xylose, glucose and arabinose) release by integrated strains H3A/EG4 # 27 or H3A is illustrated in the FIG. 11. The H3A/EG4 #27 integrated strain led to enhanced total fermentable monomer release compared to the integrated strain H3A, or compared to Eg4+1.12 mg Xyn3/g Glucan+Xylan.

### 6.7 Example 7

### Purified T. Reesei EG4 Leads to Glucose Release in Dilute Ammonia Pretreated Corncob

The effect of purified T. reesei Eg4 on the concentration of sugars released was tested using dilute ammonia pretreated corncob in the presence or absence of 0.53 mg Xyn3 per g Glucan+Xylan. The experiments were performed as described in Example 6. Results are shown in FIG. 12.

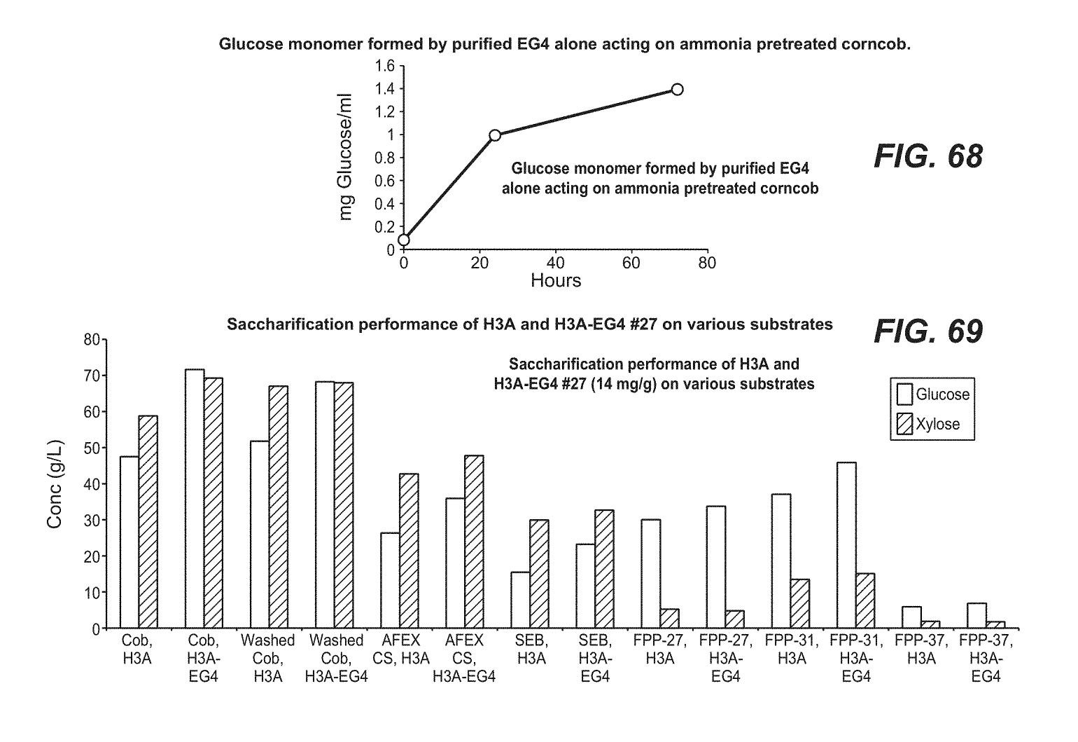

The data indicate that purified T. reesei Eg4 leads to release of glucose monomer without the action of other cellulases such as endoglucanases, cellobiohydrolases and β-glucosidases. Saccharification experiments were also conducted using dilute ammonia pretreated corncob with purified Eg4 added alone (no Xyn3 added). 3.3 μL of purified Eg4 (15.3 mg/mL) was added to 872 μL 50 mM, pH 5.0 sodium acetate buffer (included 0.01% sodium azide and 5 mM MnCl₂), 165 mg of dilute ammonia pretreated corncob (67.3% dry solids, 111 mg dry solids added) and 16.5 μL of 1 N sulfuric acid in 5 mL vials. The vials were incubated at 48° C. and rotated at 180 rpm. Periodically, 20 μL aliquots were removed, diluted 10-fold with filter sterilized double distilled water and filtered through a nylon filter before analysis for glucose released on a Dionex Ion Chromatography system. Authentic glucose solutions were used as external standards. Results are shown in FIG. 68, indicating that addition of purified Eg4 leads to release of glucose monomer from dilute ammonia pretreated corncobs over 72 h incubation at 48° C. in the absence of other cellulases or endoxylanase.

### 6.8 Example 8

### Saccharification Performance of T. Reesei Integrated Strains H3A and H3A/EG4 #27 on Various Substrates

In this experiment, fermentation broth from T. reesei integrated strain H3A or H3A/EG4 #27, dosed at 14 mg protein per g of glucan+xylan, was tested for saccharification performance on different substrates including: dilute ammonia pretreated corncob, washed dilute ammonia pretreated corncob, ammonia fiber expanded (AFEX) pretreated corn stover (CS), Steam Expanded Sugarcane Bagasse (SEB), and Kraft-pretreated paper pulps FPP27 (Softwood Industrial Unbleached Pulp delignified-Kappa 13.5, Glucan 81.9%, Xylan 8.0%, Klason Lignin 1.9%), FPP-31 (Hardwood Unbleached Pulp delignified-Kappa 10.1, Glucan 75.1%, Xylan 19.1%, Klason Lignin 2.2%), and FPP-37 (Softwood Unbleached Pulp air dried-Kappa 82, Glucan 71.4%, Xylan 8.7%, Klason Lignin 11.3%).

The saccharification reactions were set up in 25 mL glass vials with final mass of 10 g in 0.1 M Sodium Citrate Buffer, pH 5.0 and incubated at 50° C., 200 rpm for 6 d. At the end of 6 d, 100 μL aliquots were diluted 1:10 in 5 mM sulfuric acid and the samples analyzed by HPLC to determine glucose and xylose formation. Results are shown in FIG. 69.

### 6.9 Example 9

### Effect of T. Reesei EG4 on Saccharification of Acid Pretreated Corn Stover

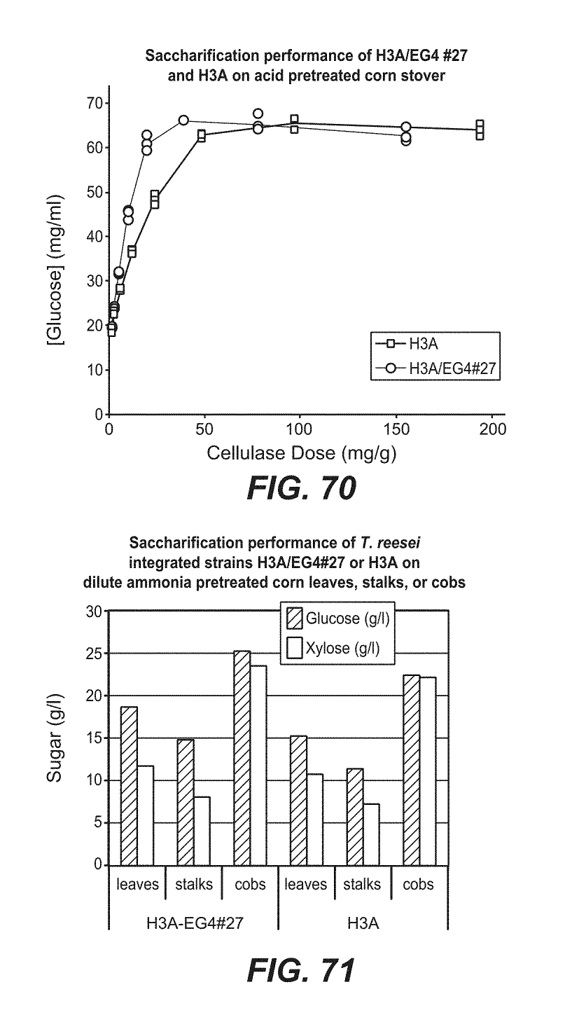

The effect of Eg4 on saccharification of acid pretreated corn stover was tested. Corn stover pretreated with dilute sulfuric acid (Schell, D J, et al., Appl. Biochem. Biotechnol. 2003, 105(1-3):69-85) was obtained from NREL, adjusted to 20% solids and conditioned to a pH 5.0 with the addition of soda ash solution. Saccharification of the pretreated substrate was performed in a microtiter plate using 20% total solids. Total protein in the fermentation broths was measured by the Biuret assay (see Example 1 above). Increasing amounts of fermentation broth from T. reesei integrated strains H3A/EG4 #27 and H3A were added to the substrate and saccharification performance was measured following incubation at 50° C., 5 d, 200 RPM shaking. Glucose formation (mg/g) was measured using HPLC. Results are shown in FIG. 70.

### 6.10 Example 10

### Saccharification Performance of T. Reesei Integrated Strains H3A and H3A/EG4 #27 on Dilute Ammonia Pretreated Corn Leaves, Stalks, and Cobs

In this experiment, saccharification performance of T. reesei integrated strains H3A and H3A/EG4 #27 was compared on dilute ammonia pretreated corn stover leaves, stalks, or cobs. Pretreatment was performed as described in WO06110901A. Five (5) g total mass (7% solids) was hydrolyzed in 20 mL vials at pH 5.3 (pH adjusted by addition of 6 N H₂SO₄) using14 mg protein per g of glucan+xylan. Saccharification reactions were carried out at 50° C. and samples analyzed by HPLC for glucose and xylose released on day 4. Results are shown in FIG. 71.

### 6.11. Example 11

### Saccharification Performance on Dilute Ammonia Pretreated Corncob in Response to Overexpressed EG4 from T. Reesei

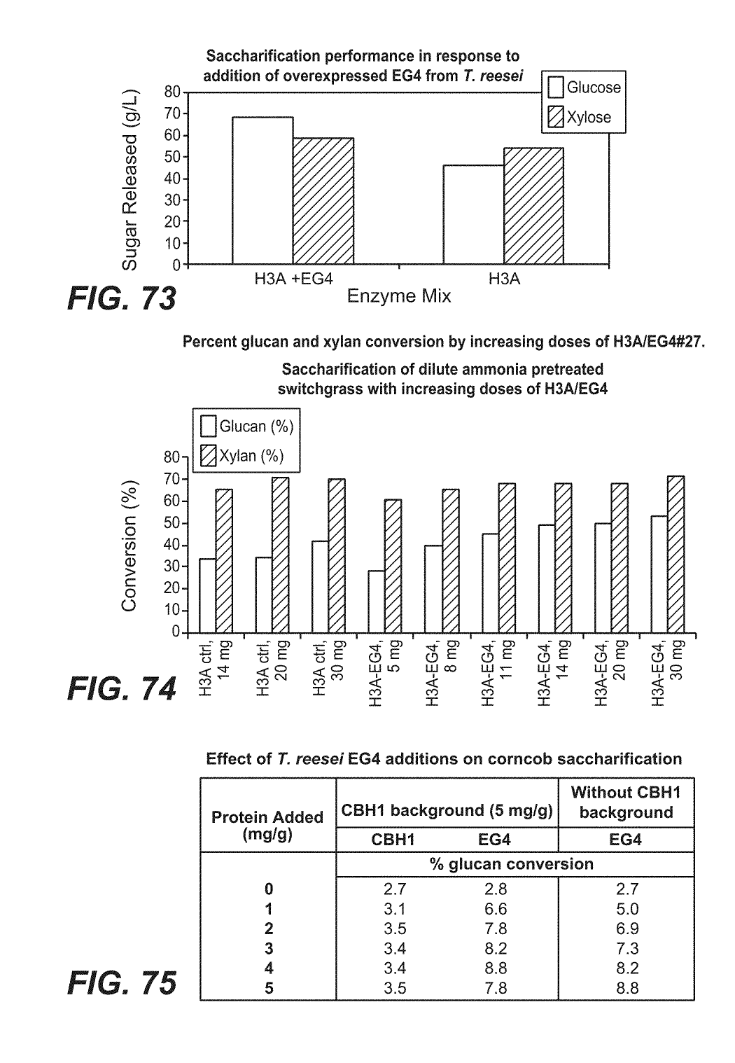

Saccharification reactions at 3 g scale were performed using dilute ammonia pretreated corncob. Sufficient pretreated cob preparation was measured into 20 mL glass vials to give 0.75 g dry solid. Enzyme preparation, 1 N sulfuric acid and 50 mM pH 5.0 sodium acetate buffer (with 0.01% sodium azide) were added to give final slurry of 3 g total reaction, 25% dry solids, pH 5.0. Extra cellular protein (fermentation broth) from the T. reesei integrated strain H3A was added at 14 mg protein/g (glucan+xylan) either with or without an additional 5% of the 14 mg protein load as the unpurified culture supernatant from a T. reesei strain (Δcbh1 Δcbh2 Δeg1 Δeg2) (See International publication WO 05/001036) over expressing Eg4. The saccharification reactions were incubated for 72 h at 50° C. Following incubation, the reaction contents were diluted 3-fold, filtered and analyzed by HPLC for glucose and xylose concentration. The results are shown in FIG. 73. Addition of Eg4 protein in the form of extracelluar protein from a T. reesei strain over expressing the protein to H3A substantially increased the release of monomer glucose and slightly increased the release of monomer xylose.

### 6.12 Example 12

### Saccharification Performance of Strain H3A/EG4 #27 on Ammonia Pretreated Switchgrass

The saccharification performance of strain H3A/EG4 #27 on dilute ammonia pretreated switchgrass (WO06110901A) at increasing protein doses was compared to that of strain H3A (18.5% solids). Pretreated switchgrass preparations were measured into 20 mL glass vials to give 0.925 g of dry solid. 1 N sulfuric acid and 50 mM pH 5.3 sodium acetate buffer (with 0.01% sodium azide) were added to give a final slurry of 5 grams total reaction. The enzyme dosages of H3A tested were 14, 20, and 30 mg/g (glucan+xylan); and the dosages of H3A-EG4 #27 were 5, 8, 11, 14, 20, and 30 mg/g (glucan+xylan). The reactions were incubated at 50° C. for 3 d. Following incubation, the reaction contents were diluted 3-fold, filtered and analyzed by HPLC for glucose and xylose concentration. The conversion of glucan and xylan were calculated based on the composition of the switchgrass substrate. The results shown in FIG. 74 indicate that the glucan conversion performance of H3A-EG4 #27 is more effective than H3A at the same enzyme dosages.

### 6.13 Example 13

### Effect of T. Reesei EG4 Additions on Corncob Saccharification and on CMC and Cellobiose Hydrolysis

6.13.1 A. Corncob Saccharification

Dilute ammonia pretreated corncob was adjusted to 20% solids, 7% cellulose and 65 mg was dispensed per well in a microtiter plate. Saccharification reactions were initiated by adding 35 μL of 50 mM sodium acetate (pH 5.0) buffer containing T. reesei CBH1 at 5 mg protein/g glucan (final) and the relevant enzymes (CBH1 or Eg4), at final concentrations of 0, 1, 2, 3, 4 and 5 mg/g glucan. An Eg4 control received only EG4 at the same doses and as such, the total added protein in these wells was less. The microtiter plates were sealed with an aluminum plate seal (E&K scientific) and mixed for 2 min at 600 rpm, 24° C. The plate was then placed in an Innova incubator at 50° C. and 200 rpm for 72 h.

At the end of 72-h saccharification, the plate was quenched by adding 100 μL of 100 mM glycine, pH 10.0. The plate was then centrifuged at 3000 rpm for 5 min. Supernatant (20 μL) was added to 100 μL of water in HPLC 96 well microtiter plate (Agilent, 5042-1385). Glucose and cellobiose concentrations were measured by HPLC using Aminex HPX-87P column (300 mm×7.8 mm, 125-0098) pre-fitted with guard column. Percent glucan conversion was calculated as 100×(mg cellobiose+mg glucose)/total glucan in substrate (FIG. 75).

6.13.2 B. CMC Hydrolysis

Carboxymethylcellulose (CMC, Sigma C4888) was diluted to 1% with 50 mM Sodium Acetate, pH 5.0. Hydrolysis reactions were initiated by separately adding each of three T. reesei purified enzymes—Eg4, EG1 and CBH1 at final concentrations of 20, 10, 5, 2.5, 1.25 and 0 mg/g to 100 μL of 1% CMC in a 96-well microtiter plate (NUNC #269787). Sodium acetate, pH 5.0 50 mM was added to each well to a final volume of 150 μL. The CMC hydrolysis reactions were sealed with an aluminum plate seal (E&K scientific) and mixed for 2 min at 600 rpm, 24° C. The plate was then placed in an Innova incubator at 50° C. and 200 rpm for 30 min.

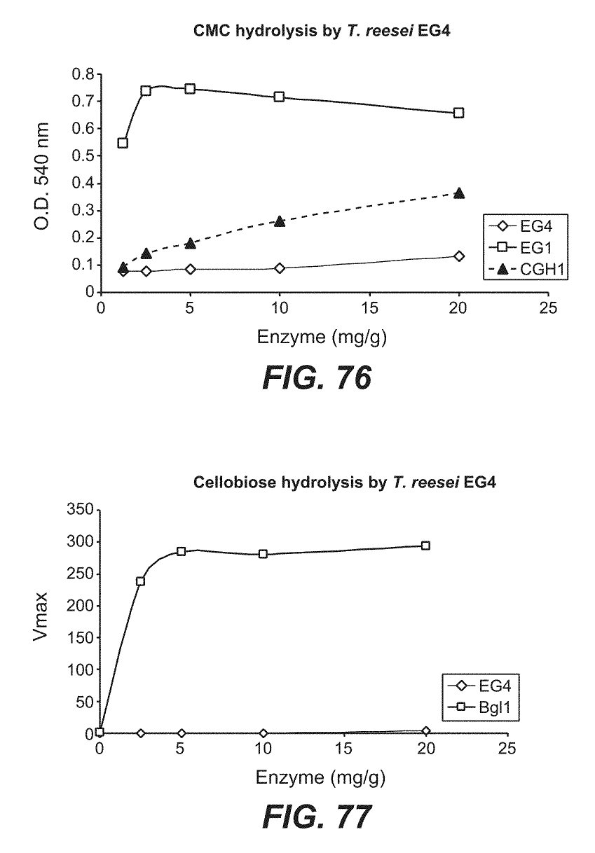

At the end of 30 min. incubation, the plate was put in ice water for 10 min. to stop the reaction, and samples were transferred to eppendorf tubes. To each tube was added 375 μL of dinitrosalicylic acid (DNS) solution (see below). Samples were then boiled for 10 min and 0.D was measured at 540 nm by SpectraMAX 250 (Molecular Devices). Results are shown in FIG. 76.

DNS Solution:

40 g 3.5-Dinitrosalicylic acid (Sigma, D0550)

8 g Phenol

2 g Sodium sulfite (Na2SO3)

800 g Na-K tartarate (Rochelle salt). Add all the above to 2 L of 2% NaOH. Stir overnight, covered with aluminum foil. Add distilled deionized water to a final volume of 4 L. Mix well. Store in a dark bottle, refrigerated.

6.13.3. C. Cellobiose Hydrolysis

Cellobiose was diluted to 5 g/L with 50 mM Sodium Acetate, pH 5.0. Hydrolysis reactions were initiated by separately adding each of two enzymes—EG4 and BGL1 at final concentrations of 20, 10, 5, 2.5, and 0 mg/g to 100 μL cellobiose solution at 5 g/L. Sodium acetate, pH 5.0 was added to each well to a final volume of 120 μL. The reaction plates were sealed with an aluminum plate seal (E&K scientific) and mixed for 2 min at 600 rpm, 24° C. The plate was then placed in an Innova incubator at 50° C. and 200 rpm for 2 h. At the end of the 2 h hydrolysis step, the plate was quenched by adding 100 μL of 100 mM glycine, pH 10.0. The plate was then centrifuged at 3000 rpm for 5 min. Glucose concentration was measured by ABTS (2,2′-azino-bis 3-ethylbenzothiazoline-6-sulfonic acid) assay (Example 1). Ten (10) μL of supernatant were added to 90 μL ABTS solution in a 96-well microtiter plate (Corning costar 9017 EIA/RIA plate, 96 well flat bottom, medium binding). O.D. 420 nm was measured by SpectraMAX 250, Molecular Devices. Results are shown in FIG. 77.

### 6.14. Example 14

### Purified Eg4 Improves Glucose Production from Dilute Ammonia Pretreated Corncob when Mixed with Various Cellulase Mixtures

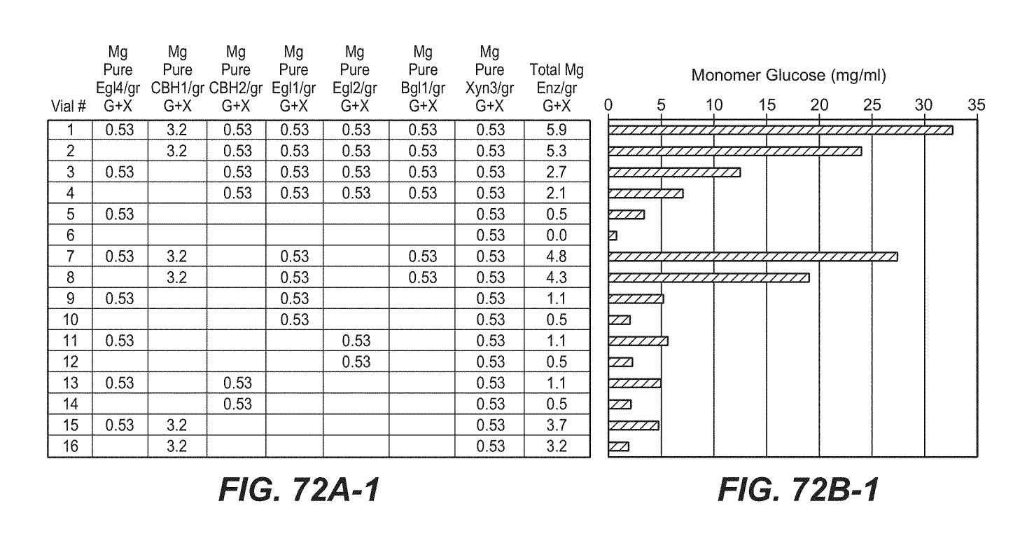

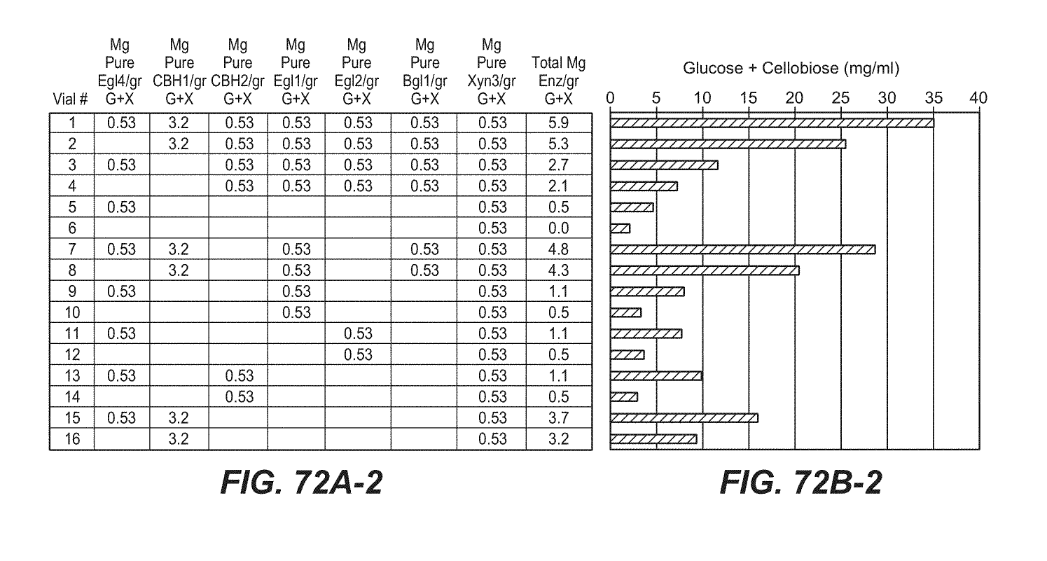

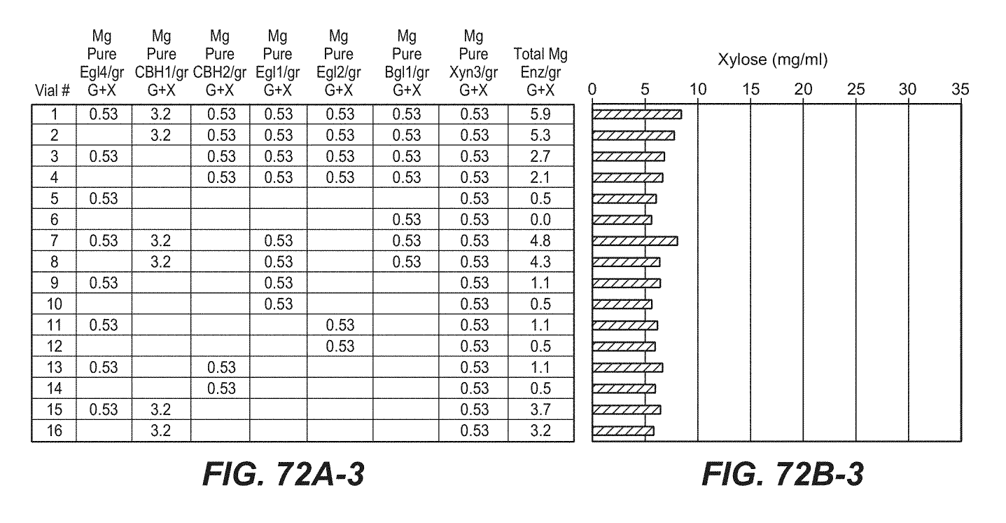

The effect of purified Eg4 combined with purified cellulases (T. reesei EG1, EG2, CBH1, CBH2, and Bgl1) on the concentration of sugars released was tested using dilute ammonia pretreated corncob in the presence of 0.53 mg T. reesei Xyn3 per g of Glucan+Xylan. 1.06-g reactions were set up in 5 mL vials containing 0.111 g dry cob solids (10.5% solids). Enzyme preparation (FIG. 72A), 1 N sulfuric acid and 50 mM pH 5.0 sodium acetate buffer (with 0.01% sodium azide and 5 mM MnCl₂) were added to give the final reaction weight. The reaction vials were incubated at 48° C. with 180 rpm rotation. After 72 h, filtered MilliQ water was added to dilute each saccharification reaction by 5-fold. The samples were centrifuged at 14,000×g for 5 min, then filtered through a 0.22 μm nylon filter (Spin-X centrifuge tube filter, Corning Incorporated, Corning, N.Y.) and further diluted 4-fold with filtered Milli-Q water to create a final 20× dilution. Twenty (20) μL injections were analyzed by HPLC to measure the sugars released (glucose, cellobiose, and xylose).

FIG. 72B shows glucose (top graph), glucose+cellobiose (center graph), or xylose (lower graph) produced with each combination. Purified Eg4 improved the performance of individual cellulases and mixtures. When all of the purified cellulases were present, addition of 0.53 mg Eg4 per g Glucan+Xylan improved the conversion by almost 40%.

Improvement was also seen when Eg4 was added to a combination of CBH1, Egl1 and Bgl1. When individual cellulases were present with the cob, the absolute amounts of total glucose release were substantially lower than resulted from the experiment wherein combinations of cellulases were present with the cob, but in each case, the percent improvement in the presence of Eg4 was significant. Addition of Eg4 to purified cellulases resulted in the following percent improvements in total Glucose release-Bgl1 (121%), Egl2 (112%), CBH2 (239%) and CBH1 (71%). This shows that Eg4 had a significant and broad effect to improve cellulase performance on biomass.

### 6.15. Example 15

### Synergestic Effects Observed When EG4 was Mixed with CBH1, CBH2, and EG2—Substrate: Dilute Ammonia Pretreated Corncob

Dilute ammonia pretreated corncob saccharification reactions were prepared by adding enzyme mixtures as follows to corncob (65 mg per well of 20% solids, 7% cellulose) in 96-well MTPs (VWR). Eighty (80) μL of 50 mM sodium acetate (pH 5.0), 1 mg Bgl1/g glucan, and 0.5 mg Xyn3/g glucan background were also added to all wells.

To test the effect of mixing Eg4 individually with CBH1, CBH2 and EG2, each of CBH1, CBH2, and EG2 was added at 0, 1.25, 2.5, 5, 10 and 20 mg/g glucan, and EG4 was added at concentrations of 20, 18.75, 17.5, 15, 10 and 0 mg/g glucan to the respective wells, making the total proteins in individual wells 20 mg/g glucan. The control wells received only CBH1 or CBH2 or EG2 or EG4 at the same doses, as such the total added proteins in these wells were less than 20 mg/g.

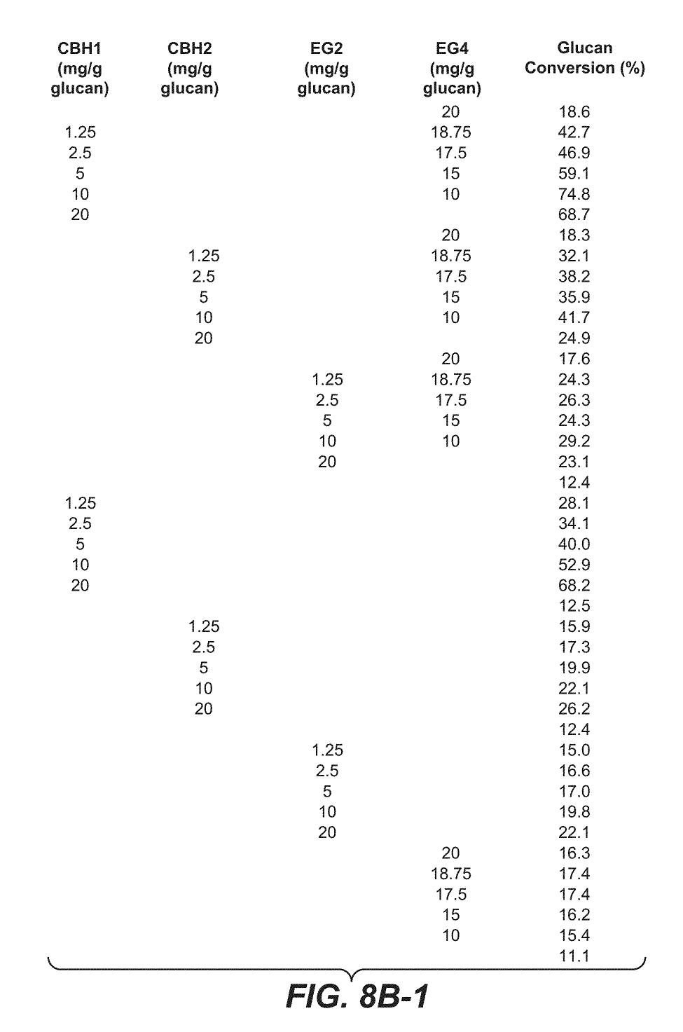

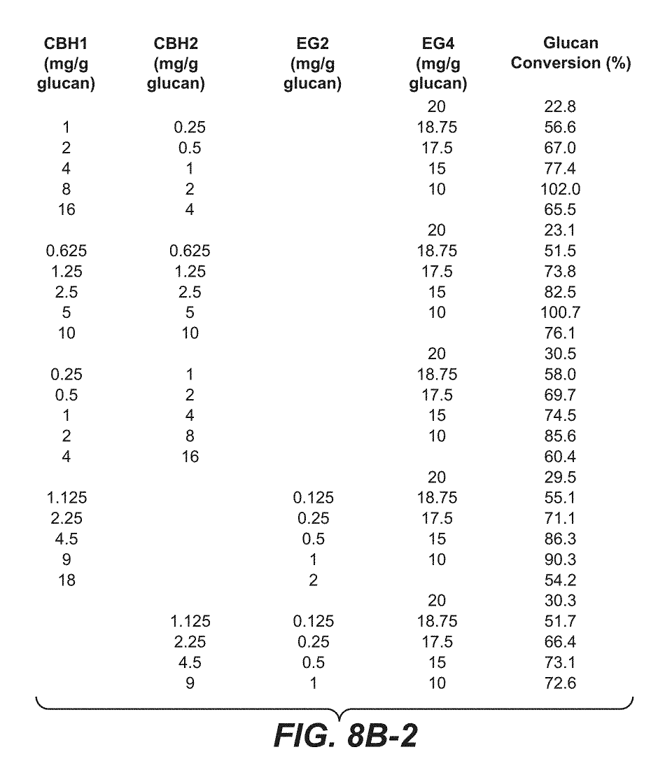

To test the effect of Eg4 on combinations of cellulases, mixtures of CBH1, CBH2 and EG2 at different ratios (see, FIG. 8A) were added at 0, 1.25, 2.5, 5, 10 and 20 mg protein/g glucan, and EG4 was added to the mixtures at concentrations of 20, 18.75, 17.5, 15, 10 and 0 mg protein/g glucan, such that the total proteins in individual wells was 20 mg protein/g glucan. As above, control wells received only one added protein so the total protein addition was less than 20 mg protein/g.

The corncob saccharification reactions were sealed with an aluminum plate seal (E&K scientific) and mixed for 2 min at 600 rpm, 24° C. The plate was then placed in an Innova 44 incubator shaker (New Brunswick Scientific) at 50° C. and 200 rpm for 72 h. At the end of the 72-h saccharification step, the plate was quenched by adding 100 μL of 100 mM glycine, pH 10.0. The plate was then centrifuged at 3000 rpm for 5 min (Rotanta 460R Centrifuge, Hettich Zentrifugen). Twenty (20) μL of supernatant was added to 100 μL of water in an HPLC 96-well microtiter plate (Agilent, 5042-1385). Glucose and cellobiose concentrations were measured by HPLC using an Aminex HPX-87P column (300 mm×7.8 mm, 125-0098) and guard column (BioRad).

The results were indicated in the table of FIG. 8B, wherein % glucan conversion is defined as % (glucose+cellobiose)/total glucan.

This experiment indicates that Eg4, when added to a CBH1, CBH2 and/or EG2, was beneficial in improving saccharification of dilute ammonia pretreated corncob. Indeed, a synergistic effect was observed, especially when Eg4 was added into a mixture comprising CBH2. Moreover, the highest improvement was observed when Eg4 and the other enzyme (CBH1, CBH2, or EG2) were added to the saccharification mixture in an equal amount. It was also observed that the effect of Eg4 is substantial on the CBH1 and CBH2 mixture. The optimum improvement by Eg4 was observed when the amount of Eg4 to CBH1 and CBH2 was 1:1. Results are indicated in FIG. 8B.

### 6.16. Example 16

### EG4 Improves Saccharification Performance of Various Hemicellulase Compositions

The total protein concentration of commercial cellulase enzyme preparations Spezyme® CP, Accellerase®1500, and Accellerase®DUET (Genencor Division, Danisco US) were determined by the modified Biuret assay (described herein).

Purified T. reesei EG4 was added to each enzyme preparation, and the samples were then assayed for saccharification performance using a 25% solids loading of dilute ammonia pretreated corncob, at a dose of 14 mg of total protein per g of substrate glucan and xylan (5 mg EG4 per g of glucan and xylan, plus 9 mg whole cellulase per g of glucan and xylan).

The saccharification reaction was carried out using 5 g of total reaction mixture in a 20 mL vial at pH 5, with incubation at 50° C. in a rotary shaker set to 200 rpm for 7 d. The saccharification samples were diluted 10× with 5 mM sulfuric acid, filtered through a 0.2 μm filter before injection into the HPLC. HPLC analysis was performed using a BioRad Aminex HPX-87H ion exclusion column (300 mm×7.8 mm).

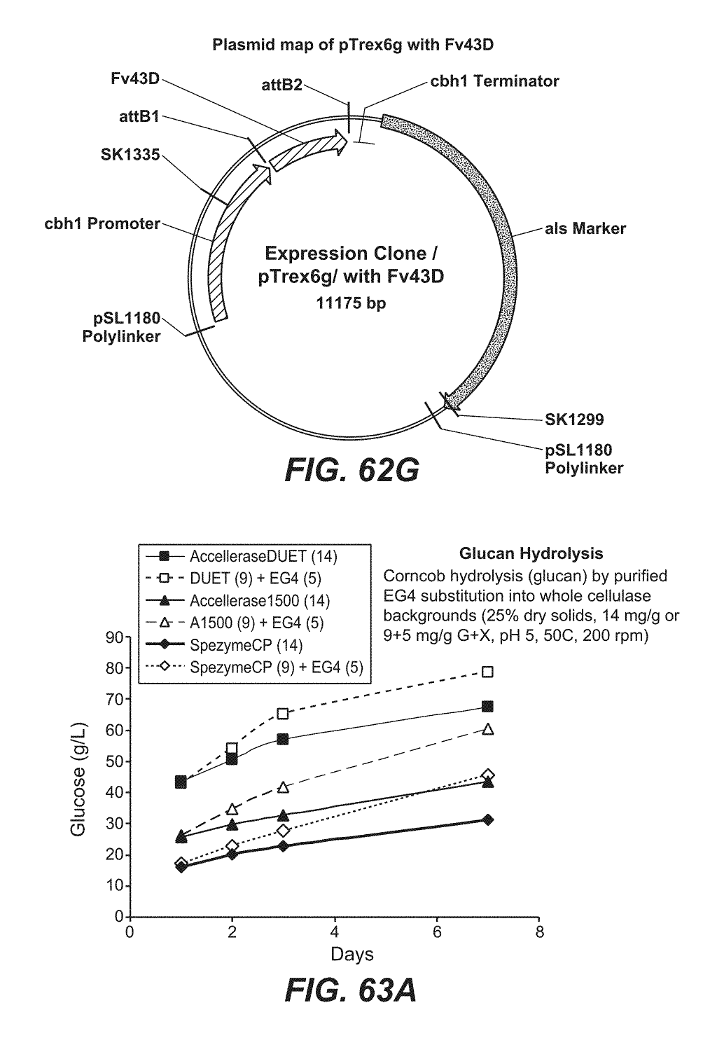

Substitution of purified Eg4 into whole cellulases improved glucan conversion in all tested cellulase products as illustrated in FIG. 63A. As illustrated in FIG. 63B, xylan conversion did not appear to be affected by the Eg4 substitution.

### 6.17 Example 17

### Cloning, Expression and Purification of Fv3C

6.17.1. A. Cloning and Expression of Fv3C

Fv3C sequence (SEQ ID NO:60) was obtained by searching for GH3 β-glucosidase homologs in the Fusarium verticillioides genome in the Broad Institute database (http://www.broadinstitute.org/) The Fv3C open reading frame was amplified by PCR using genomic DNA from Fusarium verticillioides as the template. The PCR thermocycler used was DNA Engine Tetrad 2 Peltier Thermal Cycler (Bio-Rad Laboratories). The DNA polymerase used was PfuUltra II Fusion HS DNA Polymerase (Stratagene). The primers used to amplify the open reading frame were as follows:

Forward primer MH234
(SEQ ID NO: 145)
(5′-CACCATGAAGCTGAATTGGGTCGC-3′)
Reverse primer MH235
(SEQ ID NO: 146)
(5′-TTACTCCAACTTGGCGCTG-3′)

The forward primers included four additional nucleotides (sequences—CACC) at the 5′-end to facilitate directional cloning into pENTR/D-TOPO (Invitrogen, Carlsbad, Calif.). The PCR conditions for amplifying the open reading frames were as follows: Step 1: 94° C. for 2 min. Step 2: 94° C. for 30 sec. Step 3: 57° C. for 30 sec. Step 4: 72° C. for 60 sec. Steps 2, 3 and 4 were repeated for an additional 29 cycles. Step 5: 72° C. for 2 min. The PCR product of the Fv3C open reading frame was purified using a Qiaquick PCR Purification Kit (Qiagen). The purified PCR product was initially cloned into the pENTR/D-TOPO vector, transformed into TOP10 Chemically Competent E. coli cells (Invitrogen) and plated on LA plates containing 50 ppm kanamycin. Plasmid DNA was obtained from the E. coli transformants using a QIAspin plasmid preparation kit (Qiagen). Sequence confirmation for the DNA inserted in the pENTR/D-TOPO vector was obtained using M13 forward and reverse primers and the following additional sequencing primers:

MH255
(SEQ ID NO: 147)
(5′-AAGCCAAGAGCTTTGTGTCC-3′)
MH256
(SEQ ID NO: 148)
(5′-TATGCACGAGCTCTACGCCT-3′)
MH257
(SEQ ID NO: 149)
(5′-ATGGTACCCTGGCTATGGCT-3′)
MH258
(SEQ ID NO: 150)
(5′-CGGTCACGGTCTATCTTGGT-3′)

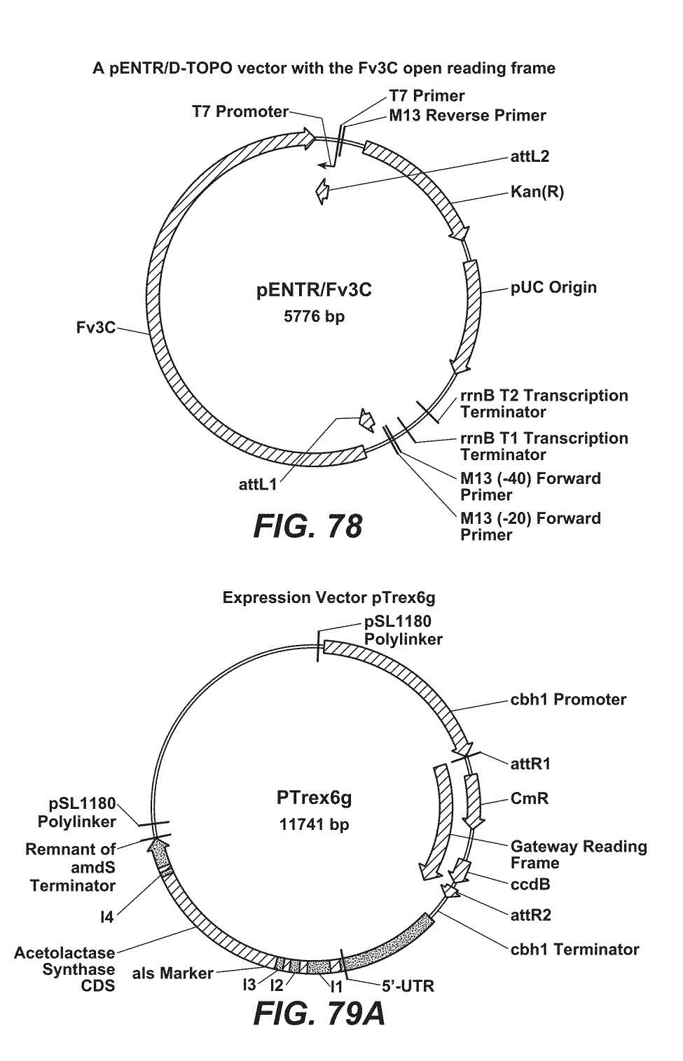

A pENTR/D-TOPO vector with the correct DNA sequence of the Fv3C open reading frame (FIG. 78) was recombined with the pTrex6g (FIG. 79A) destination vector using LR clonase® reaction mixture (Invitrogen).

The product of the LR clonase® reaction was subsequently transformed into TOP10 Chemically Competent E. coli cells (Invitrogen), which were then plated onto LA plates containing 50 ppm carbenicillin. The resulting pExpression construct was pTrex6g/Fv3C (FIG. 79B) containing the Fv3C open reading frame and the T. reesei mutated acetolactate synthase selection marker (als). DNA of the pExpression construct containing the Fv3C open reading frame was isolated using a Qiagen miniprep kit and used for biolistic transformation of T. reesei spores.

Biolistic transformation of T. reesei with the pTrex6g expression vector containing the appropriate Fv3C open reading frame was performed. Specifically, a T. reesei strain wherein cbh1, cbh2, eg1, eg2, eg3, and bgl1 have been deleted (i.e., the hexa-delete strain, see, International Publication WO 05/001036) was transformed by helium-bombardment using a Biolistic® PDS-1000/he Particle Delivery System (Bio-Rad) following the manufacturer's instructions (see US 2006/0003408). Transformants were transferred to fresh chlorimuron ethyl selection plates. Stable transformants were inoculated into filter microtiter plates (Corning), containing 200 μL/well of a glycine minimal medium (containing 6.0 g/L glycine; 4.7 g/L (NH₄)₂SO₄; 5.0 g/L KH₂PO₄; 1.0 g/L MgSO₄.7H₂O; 33.0 g/L PIPPS, pH 5.5) with post sterile addition of ˜2% glucose/sophorose mixture as the carbon source, 10 mL/L of 100 g/L of CaCl₂, 2.5 mL/L of a 400× T. reesei trace elements solution containing: 175 g/L Citric acid anhydrous; 200 g/L FeSO₄.7H₂O; 16 g/L ZnSO₄.7H₂O; 3.2 g/L CuSO₄.5H₂O; 1.4 g/L MnSO₄.H₂O; 0.8 g/L H₃BO₃. Transformants were grown in the liquid culture for five days. In a 28° C. incubator. The supernatant samples from the filter microtiter plate were collected on a vacuum manifold. Supernatant samples were run on 4-12% NuPAGE gels and stained using the Simply Blue stain (Invitrogen).

6.17.2. B. Purification of Fv3C

Fv3C, from shake flask concentrate, was dialyzed overnight against a 25 mM TES buffer, pH 6.8. The dialyzed enzyme solution was loaded on a SEC HiLoad Superdex 200 Prep Grade cross-linked agarose and dextran column (GE Healthcare) at a flow rate of 1 mL/min, which had been pre-equilibrated with 25 mM TES, 0.1 M sodium chloride at pH 6.8. SDS-PAGE was used to identify and ascertain the presence of Fv3C in the fractions from the SEC separation. Fractions containing Fv3C were pooled and concentrated. The SEC purification was also used to separate Fv3C from low and high molecular mass contaminants. The purity of the enzyme preparation was determined using Coomassie blue stained SDS/PAGE. The SDS/PAGE showed a single major band at 97 kDa.

6.17.3. C. Alternative Translation of Fv3C

For expression of the Fv3C gene, the genomic sequence containing the ORF as annotated in the Fusarium database was used. (www.broadinstitute.org/annotation/genome/fusarium_group/MultiHome.html). The predicted coding region contains 3 introns, with the first intron interrupting the signal peptide sequence FIG. 80.

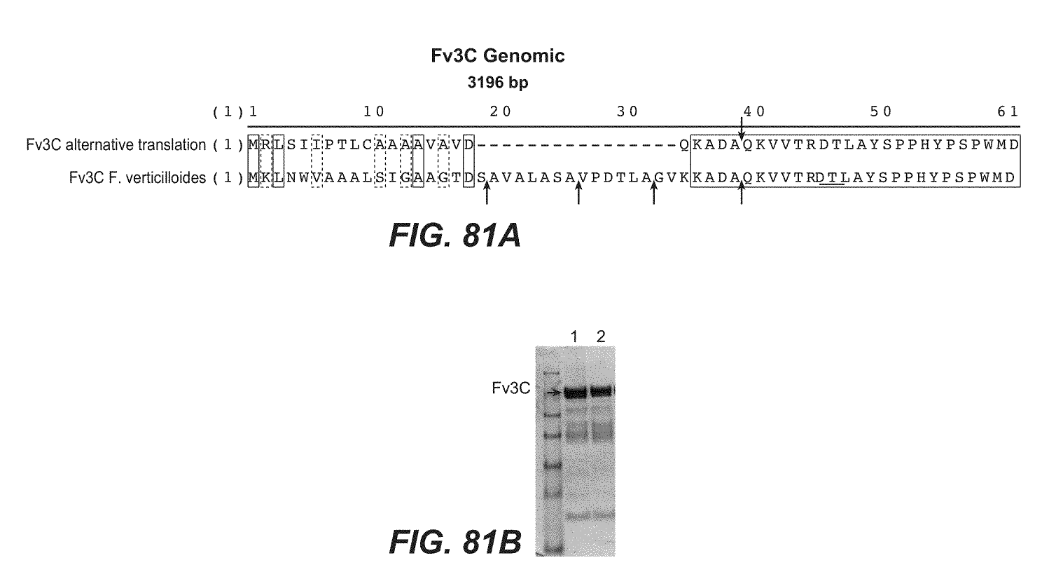

At its 3′ end, the first intron contained an alternative ORF, in frame with the mature sequence, which is also predicted to code for a signal peptide (FIG. 80). In both translations, the start site for the mature protein (underlined in FIG. 81A), as determined by N-terminal sequence analysis, started downstream from both putative signal peptide cleavage sites (shown by arrows). It was shown that Fv3C could be effectively expressed by using either of the ATGs as putative starts of translation (FIG. 81B).

### 6.18. Example 18

### β-Glucosidase Activity on Cellobiose and CNPG

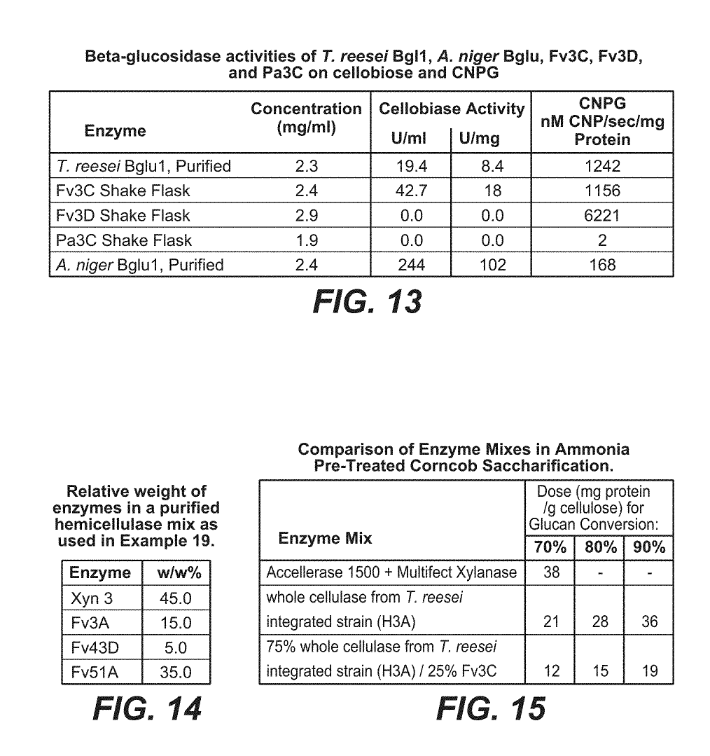

In this experiment, the β-glucosidase activities of T. reesei Bgl1 (Tr3A), A. niger Bglu (An3A) (Megazyme International Ireland Ltd., Wicklow, Ireland), Fv3C (SEQ ID NO:60), Fv3D (SEQ ID NO:58), and Pa3C (SEQ ID NO:44) on cellobiose and CNPG were tested. T. reesei Bgl1, and A. niger Bglu (“An3A”) were purified proteins. Fv3C, Fv3D and Pa3C were not purified proteins. They were expressed in a T. reesei hexa-delete strain (see above), but some background protein activities were still present. As shown in FIG. 13, Fv3C was found to have about twice the activity of T. reesei Bgl1 on cellobiose, whereas A. niger Bglu was found to be about 12 times more active than T. reesei Bgl1.

Activity of Fv3C on the CNPG substrate was about equal to that of T. reesei Bgl1, but the activity of A. niger Bglu was about 14% of the activity of T. reesei Bglu1 (FIG. 13). Fv3D, another Fusarium verticillioides β-glucosidase expressed similarly to Fv3C, had no measurable cellobiase activity, yet its activity on CNPG was about 5 times that of T. reesei Bgl1. In addition, a similarly produced Podospora anserina β-glucosidase homolog Pa3C had no measurable activity on cellobiose or CNPG substrate. These studies demonstrate that the activities of Fv3C on cellobiose and CNPG were due to the molecule itself and were not due to background protein activities.

### 6.19. Example 19

### Fv3C Saccharification on Various Biomass Substrates

6.19.1. A. Fv3C Saccharification Performance on PASC

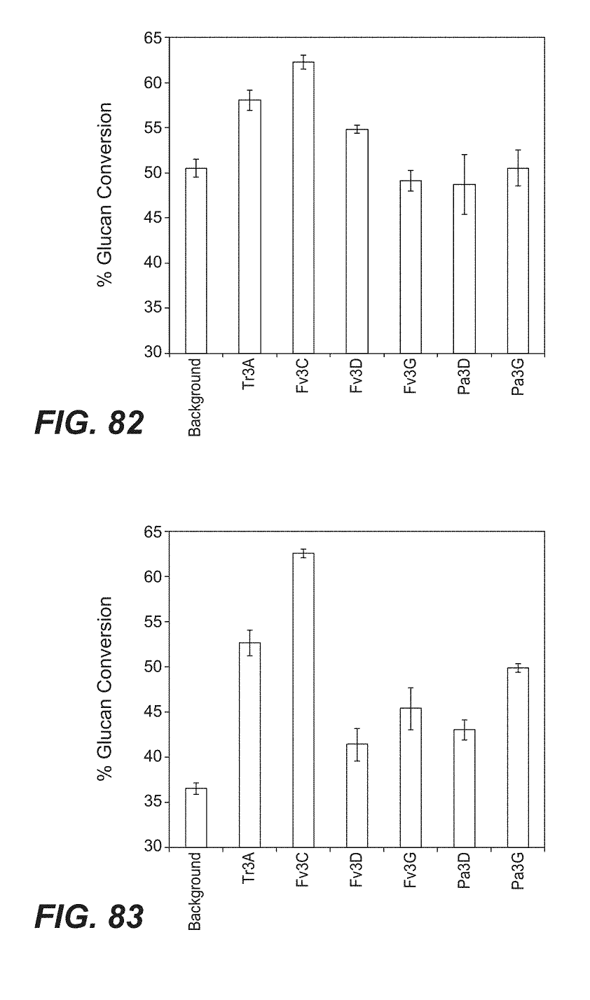

In this experiment, the ability of T. reesei Bgl1, Fv3C, and several Fv3C homologs to enhance PASC saccharification was tested. Twenty (20) μL of each β-glucosidase was added in an amount of 5 mg protein/g cellulose to a 10 mg protein/g cellulose loading of whole cellulase from a T. reesei bgl1-reduced strain, in a 96-well HPLC plate. One hundred and fifty (150) μL of a 0.7% solids slurry of PASC was added to each well and the plates were covered with aluminum plate sealers and placed in an incubator set at 50° C. for 2 h with shaking. The reaction was terminated by adding 100 μL of a 100 mM glycine buffer, pH10 to individual wells. After thorough mixing, the plates were centrifuged and the supernatants were diluted 10 fold into another HPLC plate, which contained 100 μL of 10 mM glycine, pH 10 in individual wells. The concentrations of soluble sugars produced were measured using HPLC (FIG. 82).

It was observed that the Fv3C-containing mixture yielded a higher proportion of glucose than the T. reesei Bgl1-containing mixture under the same conditions. This indicated that Fv3C has a higher cellobiase activity than T. reesei Bgl1 (see also FIG. 13). Fv3G, Pa3D and Pa3G had no observable effect on PASC hydrolysis, which indicated the lack of contribution from the hexa-delete background (in which the various Fv3C homologs were cloned and expressed) on PASC hydrolysis.

6.19.2. B. Fv3C Saccharification Performance on Dilute Acid Pretreated Cornstover (PCS)

In this experiment, the abilities of T. reesei Bgl1, Fv3C, and several Fv3C homologs to enhance PCS saccharification at 13% solids was tested using the method described in the Microtiter plate Saccharification assay (supra). For each enzyme tested, 5 mg protein/g cellulose of β-glucosidase was added to 10 mg protein/g cellulose of a whole cellulase derived from a T. reesei-Bgl1 reduced strain.

Specifically, 5 mg protein/g cellulose of each of the β-glucosidases (Bgl1, Fv3C, and homologs) was added to 10 mg protein/g cellulose of a whole cellulase derived from a T. reesei Bgl1 reduced strain, or to 8 mg protein/g cellulose of a purified hemicellulase mixture (the components of which are indicated in FIG. 14). The % glucan conversion was measured after the enzymatic mixtures were incubated with the substrate for 2 d at 50° C.

Results are shown in FIG. 83. Fv3C imparted a clear benefit in terms of %glucan conversion as compared to T. reesei Bgl1. In addition, Fv3C also promoted higher glucose and total sugar yields than T. reesei Bgl1.

The results indicated limited if any contribution from host cell background proteins.

### 6.19.3. C. Fv3C Saccharification Performance on Ammonia Pretreated Corncob

In this experiment, the ability of T. reesei Bgl1, Fv3C, and A. niger Bglu (An3A) to enhance saccharification of ammonia pre-treated corncob at 20% solids was tested in accordance with the method described in the Microtiter Plate Saccharification assay (supra).

Specifically, 5 mg protein/g cellulose of β-glucosidases (e.g., T. reesei Bgl1, Fv3C, and homologs) were added to the dilute ammonia pretreated corncob substrate, and 10 mg protein/g cellulose of whole cellulase derived from a T. reesei Bgl1-reduced strain was also added. In addition, 8 mg protein/g cellulose of a purified hemicellulase mix (FIG. 14) containing Xyn3, Fv3A, Fv43D and Fv51A was also added to the mixture. The %glucan conversion was measured after the enzyme mixtures were incubated with the substrate for 2 d at 50° C.

Results are shown in FIG. 84. Fv3C appeared to have performed better than the other β-glucosidases, including T. reesei Bgl1 (Tr3A). It was additionally observed that A. niger Bglu (An3A) additions to the enzyme mixture to a level above 2.5 mg/g cellulose impeded saccharification.

6.19.4. D. Fv3C Saccharification Performance on Sodium Hydroxide (NaOH) Pretreated Corncob

To test the effect of various substrate pretreatment methods on Fv3C performance, the ability of T. reesei Bgl1 (also termed Tr3A), Fv3C, and A. niger Bglu (An3A) to enhance saccharification of NaOH pretreated corncob at 12% solids was measured in accordance with the method described in the Microtiter plate Saccharification assay (supra). Sodium hydroxide pretreatment of corncob was performed as follows: 1,000 g of corncob was milled to about 2 mm in size, and was then suspended in 4 L of 5% aqueous sodium hydroxide solution, and heated to 110° C. for 16 h. The dark brown liquid was filtered hot under laboratory vacuum. The solid residue on the filter was washed with water until no more color eluted. The solid was dried under laboratory vacuum for 24 h. One hundred (100) g of the sample was suspended in 700 mL water and stirred. The pH of the solution was measured to be 11.2. Aqueous citric acid solution (10%) was added to lower the pH to 5.0 and the suspension was stirred for 30 min. The solid was then filtered, washed with water, and dried under vacuum at room temperature for 24 h. After drying, 86.2 g of polysaccharide enriched biomass was obtained. The moisture content of this material was about 7.3 wt %. Glucan, xylan, lignin and total carbohydrate content were measured before and after sodium hydroxide treatment, as determined by the NREL methods for carbohydrate analysis. The pretreatment resulted in delignification of the biomass while maintaining a glucan/xylan weight ration within 15% of that for the untreated biomass.

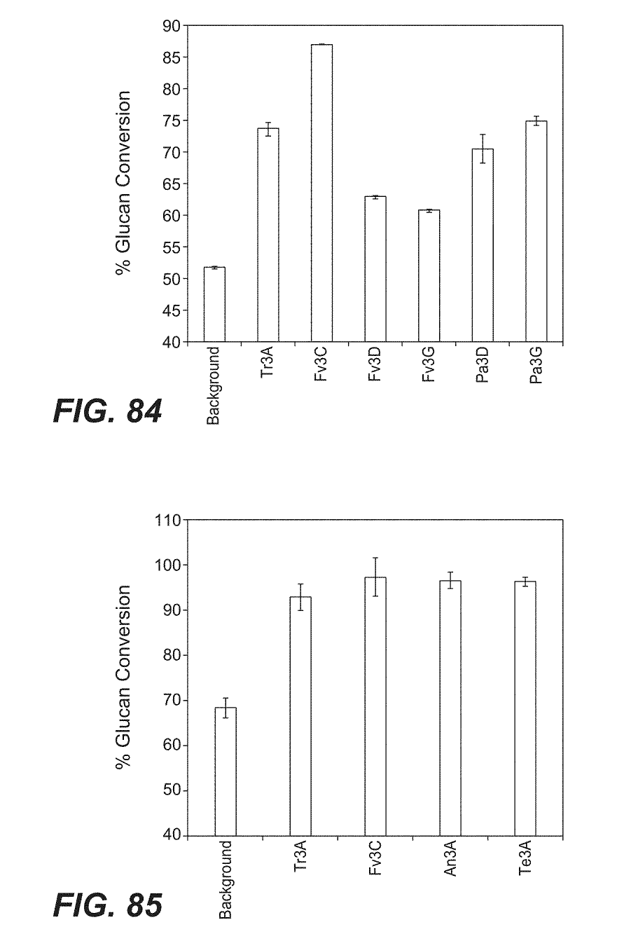

Five (5) mg protein/g cellulose of β-glucosidases (Fv3C and homologs) were added to the NaOH pretreated substrate with 8.7 mg protein/g cellulose of a whole cellulase derived from an integrated T. reesei strain H3A specifically selected for its low level of Bgl1 expression (“the H3A-5 strain”). No additional purified hemicellulases (e.g., the mixture of FIG. 14) were added to the whole cellulase background in this experiment. The %glucan conversion was measured after the enzyme mixtures were incubated with the substrate for 2 d at 50° C. The results are shown in FIG. 85. It was observed that Fv3C performed somewhat better than the other β-glucosidases, including T. reesei Bgl1 (Tr3A), An3A, and Te3A. It has also been observed that additions of A. niger Bglu (An3A) to the level above 4 mg/g cellulose resulted in lower conversion.

6.19.5. E. Fv3C Saccharification Performance on Dilute Ammonia-Pretreated Switchgrass

In this experiment, the ability of T. reesei Bgl1, Fv3C, and A. niger Bglu (An3A) to enhance saccharification of dilute ammonia pretreated switchgrass at 17% solids was tested in accordance with the method described in the Microtiter Plate Saccharification assay (supra). Dilute ammonia pretreated switchgrass was obtained from DuPont. The composition was determined using the National Renewable Energy Laboratory (NREL) procedure, (NREL LAP-002),available at: www.nrel.gov/biomass/analytical_procedures.html.

The composition based on dry weight was glucan (36.82%), xylan (26.09%), arabinan (3.51%), lignin-acid insoluble (24.7%), and acetyl (2.98%). This raw material was knife milled to pass a 1 mm screen. The milled material was pretreated at ˜160° C. for 90 min in the presence of 6 wt % (of dry solids) ammonia. Initial solids loading was about 50% dry matter. The treated biomass was stored at 4° C. before use.

In this experiment, 5 mg protein/g cellulose of β-glucosidases (e.g., T. reesei Bgl1, Fv3C, and homologs) were added to the dilute ammonia pretreated switchgrass, in the presence of 10 mg protein/g cellulose of a whole cellulase derived from an integrated T. reesei strain (H3A) selected for low β-glucosidase expression. The % glucan conversion was measured after the enzyme mixtures were incubated with the substrate for 2 d at 50° C. and the results are indicated in FIG. 86.

Fv3C performed better than the T. reesei Bgl1 and the A. niger Bglu with the switchgrass substrate.

6.19.6. F. Fv3C Saccharification Performance on AFEX Cornstover

In this experiment, the ability of T. reesei Bgl1, Fv3C, and A. niger Bglu to enhance saccharification of AFEX cornstover at 14% solids was tested in accordance to the method described in the Microtiter Plate Saccharification assay (supra). AFEX pretreated corn stover was obtained from Michigan Biotechnology Institute International (MBI). The composition of the corn stover was determined with the National Renewable Energy Laboratory (NREL) procedure LAP-002, www.nrel.gov/biomass/analytical_procedures.html. The composition based on dry weight was glucan (31.7%), xylan (19.1%), galactan (1.83%), and arabinan (3.4%). This raw material was AFEX treated in a 5 gallon pressure reactor (Parr) at 90° C., 60% moisture content, 1:1 biomass to ammonia loading, and for 30 min. The treated biomass was removed from the reactor and left in a fume hood to evaporate the residual ammonia. The treated biomass was stored at 4° C. before use.

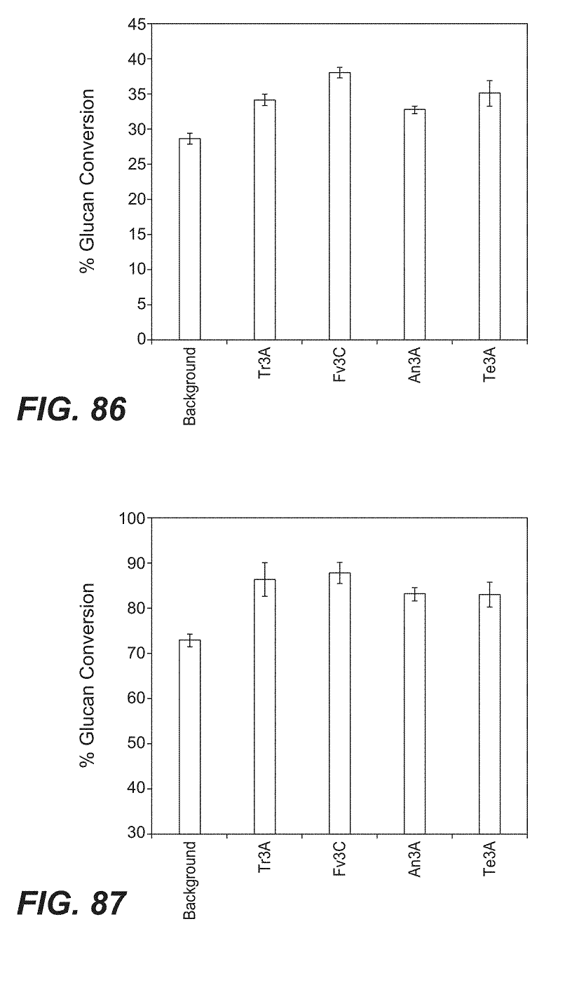

In this experiment, 5 mg protein/g cellulose of β-glucosidases (Fv3C and homologs) were added to the pretreated substrate, in the presence of 10 mg protein/g cellulose of whole cellulase derived from a low β-glucosidase expressing integrated T. reesei strain. The % glucan conversion was measured after the enzyme mixtures were incubated with the substrate for 2 d at 50° C., and the results were indicated in FIG. 87.

Fv3C performed better than T. reesei Bgl1 at glucan conversion. It was also noted that 10 mg/g cellulose of Fv3C and 10 mg/g cellulose of H3A whole cellulase under the above conditions resulted in a complete or an apparently complete glucan conversion. At levels below 1 mg/g cellulose, the A. niger Bglu (An3A) appeared to give higher glucose and total glucan conversions than that of Fv3C and T. reesei Bgl1, but at levels above 2.5 mg/g cellulose, it was observed that Fv3C and T. reesei Bgl1 had higher glucose and glucan conversion than A. niger Bglu (An3A).

### 6.20 Example 20

### Optimization of Fv3C to Whole Cellulase Ratio for Ammonia Pretreated Corncob Saccharification

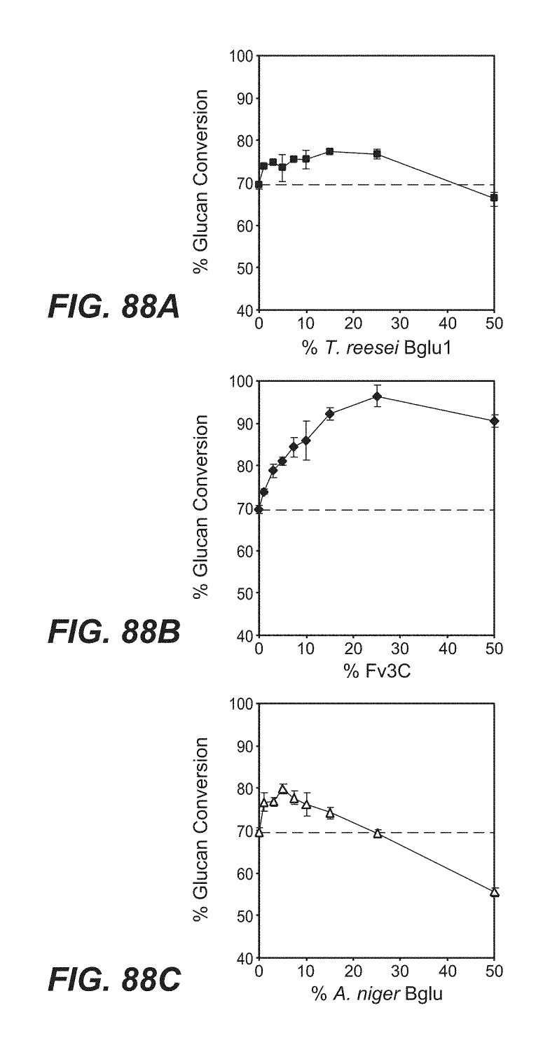

In this experiment, the ratio of Fv3C to whole cellulase was varied to determine the optimal ratio of Fv3C to whole cellulase in a hemicellulase composition. Ammonia pretreated corncob was used as substrate. The ratio of β-glucosidases (e.g., T. reesei Bgl1 (Tr3A), Fv3C, A. niger Bglu) to the whole cellulase derived from T. reesei integrated strain (H3A) was varied from 0 to 50% in the hemicellulase composition. The mixtures were added to hydrolyze ammonia pre-treated corncob at 20% solids at 20 mg protein/g cellulose. The results are shown in FIGS. 88A-88C.

The optimal ratio of T. reesei Bgl1 (Tr3A) to whole cellulase was broad, centering at about 10%, with the 50% mixture yielding similar performance to the same loading of whole cellulase alone. In contrast, the A. niger Bglu (or An3A) reached optimum at about 5%, and the peak was sharper. At the peak/optimum level, A. niger Bglu (or An3A) gave higher conversion than the optimal mix comprising T. reesei Bgl1 (Tr3A).

The optimal ratio of Fv3C to whole cellulase was determined to be about 25%, with the mixture yielding over 96% glucan conversion at 20 mg total protein/g cellulose. Thus, 25% of the enzymes in whole cellulase can be replaced with a single enzyme, Fv3C, resulting in improved saccharification performance.

### 6.21 Example 21

### Saccharification of Ammonia Pretreated Corncob by Different Enzyme Blends

A 25% Fv3C/75% whole cellulase from T. reesei integrated strain (H3A) mixture was compared with other high performing cellulase mixtures in a dose response experiment.

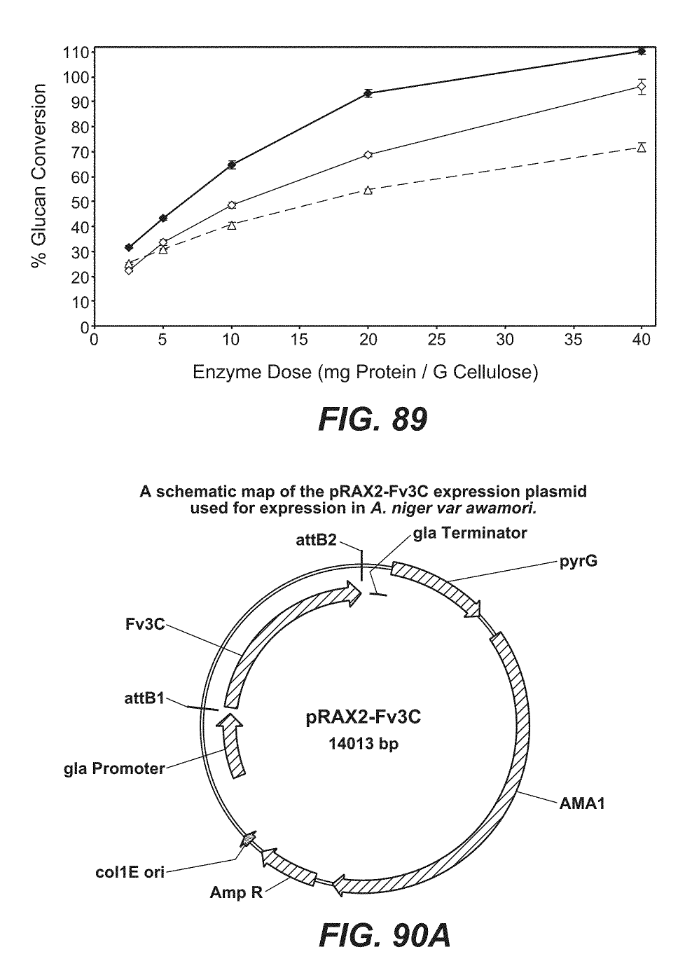

Whole cellulase from T. reesei integrated strain (H3A) alone, 25% Fv3C/75% whole cellulase from T. reesei integrated strain (H3A) mixture, and Accellerase® 1500+Multifect® Xylanase were compared for their saccharification performances on dilute ammonia pre-treated corncob at 20% solids. The enzyme blends were dosed from 2.5 to 40 mg protein/g cellulose in the reaction. Results are shown in FIG. 89.

The 25% Fv3C/75% whole cellulase from T. reesei integrated strain (H3A) mixture performed dramatically better than the Accellerase® 1500+Multifect® Xylanase blend, and showed a substantial improvement over the whole cellulase from T. reesei integrated strain (H3A). The dose required for 70, 80 or 90% glucan conversion from each enzyme mix is listed in FIG. 15. At 70% glucan conversion, the 25% Fv3C/75% whole cellulase from T. reesei integrated strain (H3A) mixture gave a 3.2 fold dose reduction when compared to the Accellerase® 1500+Multifect® Xylanase blend. At 70, 80 or 90% glucan conversion, the 25% Fv3C/75% whole cellulase from T. reesei integrated strain (H3A) mixture required about 1.8-fold less enzyme than the whole cellulase from T. reesei integrated strain (H3A) alone.

### 6.22 Example 22

### Expression of Fv3C in Aspergillus Niger Strain

To express Fv3C in A. niger, the pEntry-Fv3C plasmid was recombined with a destination vector pRAXdest2, as described in U.S. Pat. No. 7,459,299, using the Gateway LR recombination reaction (Invitrogen). The expression plasmid contained the Fv3C genomic sequence under the control of the A. niger glucoamylase promoter and terminator, the A. nidulans pyrG gene as a selective marker, and the A. nidulans ama1 sequence for autonomous replication in fungal cells. Recombination products generated were transformed into E. coli Max Efficiency DH5α (Invitrogen), and clones containing the expression construct pRAX2-Fv3C (FIG. 90A) were selected on 2× YT agar plates, prepared with 16 g/L Bacto Tryptone (Difco), 10 g/L Bacto Yeast Extract (Difco), 5 g/L NaCl, 16 g/L Bacto Agar (Difco), and 100 μg/mL ampicillin.

About 50-100 mg of the expression plasmid was transformed into an A. niger var awamori strain (see, U.S. Pat. No. 7,459,299). The endogenous glucoamylase glaA gene was deleted from this strain, and it carried a mutation in the pyrG gene, which allowed for selection of transformants for uridine prototrophy. A. niger transformants were grown on MM medium (the same minimal medium as was used for T. reesei transformation but 10 mM NH₄Cl was used instead of acetamide as a nitrogen source) for 4-5 d at 37° C., and a total population of spores (about 10⁶ spores/mL) from different transformation plates was used to inoculate shake flasks containing production medium (per 1L): 12 g trypton; 8 g soyton; 15 g (NH₄)₂SO₄; 12.1 g NaH₂PO₄xH₂O; 2.19 g Na₂HPO₄x2H₂O; 1 g MgSO₄x7H₂O; 1 mL Tween 80; 150 g Maltose; pH 5.8. After 3 d of fermentation at 30° C. and shaking at 200 rpm, the expression of Fv3C in transformants was confirmed by SDS-PAGE.

### 6.23. Example 23

### Construction of and Screening for Additional T. Reesei Integrated Strains

6.23.1. A. Generation of the CB #201 Strain

A T. reesei mutant strain, derived from RL-P37 (Sheir-Neiss, G. and B. S. Montenecourt, Appl. Microbiol. Biotechnol. 1984, 20:46-53) and selected for high cellulase production, was co-transformed with three hemicellulase genes (Fv3A, Fv43D, and Fv51A) from F. verticillioides. They were co-transformed by electroporation in three different combinations, which included the T. reesei egl1 promoter (Peg/1), T. reesei cbh2 promoter (Pcbh2), or T. reesei cbh1 promoter (Pcbh1) and the acetolactate synthase (a/s) marker (US2007/020484, WO 2009/114380). The three combinations were as follows: 1) Pegl1-fv51a, Pcbh2-fv43d-als, and Pegl1-fv3a, 2) Pcbh1-fv3a-als marker, Pegl1-fv51a, and Pcbh2-fv43d, and 3) Peg/1-fv51a, Pcbh1-fv43d-als and Pegl1-fv3a. Following electroporation, the transformation mixtures were plated onto selective agar containing chlorimuron ethyl. Transformants were then grown in microtiter plates as described in WO/2009/114380. The resulting transformants were screened in MTP scale corncob saccharification performance assays as previously described. The screening resulted in identification of a strain (CB #201) that showed high levels of glucose and xylose conversion.

The following primer pairs were used for amplifying the expression cassettes: Pegl1-fv51a primer pair:

SK1298
(SEQ ID NO: 151)
5′-GTAGTTATGCGCATGCTAGAC-3′
SK1289
(SEQ ID NO: 152)
5′-GTGGCTAGAAGATATCCAACAC-3′

Pcbh2-fv43d-als primer pair:

SK1438
(SEQ ID NO: 153)
5′-CGTCTAACTCGAACATCTGC-3′
SK1299
(SEQ ID NO: 154)
5′-GTAgcggccgcCTCATCTCATCTCATCCATCC-3′

Pegl1-fv3a primer pair

SK1298
(SEQ ID NO: 155)
5′-GTAGTTATGCGCATGCTAGAC-3′
SK822-
(SEQ ID NO: 156)
5′-CACGAAGAGCGGCGATTC-3′

Pcbh1-fv3α-als primer pair:

SK1335
(SEQ ID NO: 157)
5′-GCAACGGCAAAGCCCCACTTC-3′
SK1299
(SEQ ID NO: 158)
5′-GTAgcggccgcCTCATCTCATCTCATCCATCC-3′

Pcbh2-fv43d primer pair:

SK1438
(SEQ ID NO: 159)
5′-CGTCTAACTCGAACATCTGC-3′
SK1449
(SEQ ID NO: 160)
5′-CATggcgcgccCAACTGCCCGTTCTGTAGC-3′

Pcbh1-fv43d-als primer pair:

SK1335
(SEQ ID NO: 157)
5′-GCAACGGCAAAGCCCCACTTC-3′
SK1299
(SEQ ID NO: 161)
5′-GTAgcggccgcCTCATCTCATCTCATCCATCC-3′

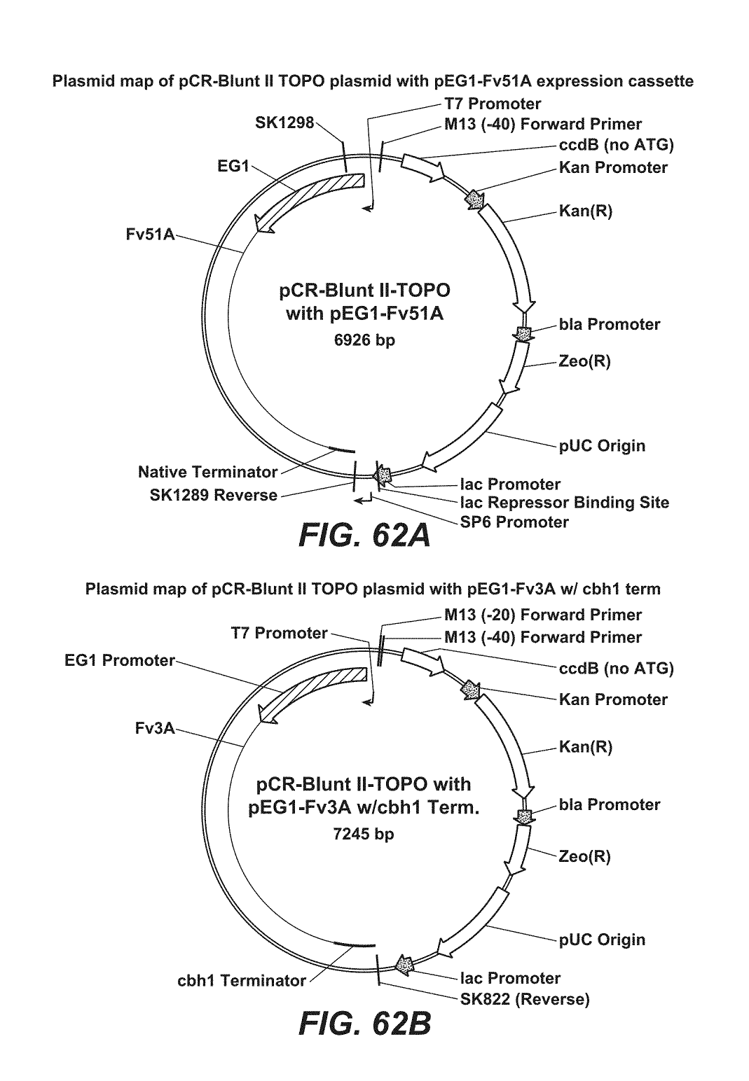

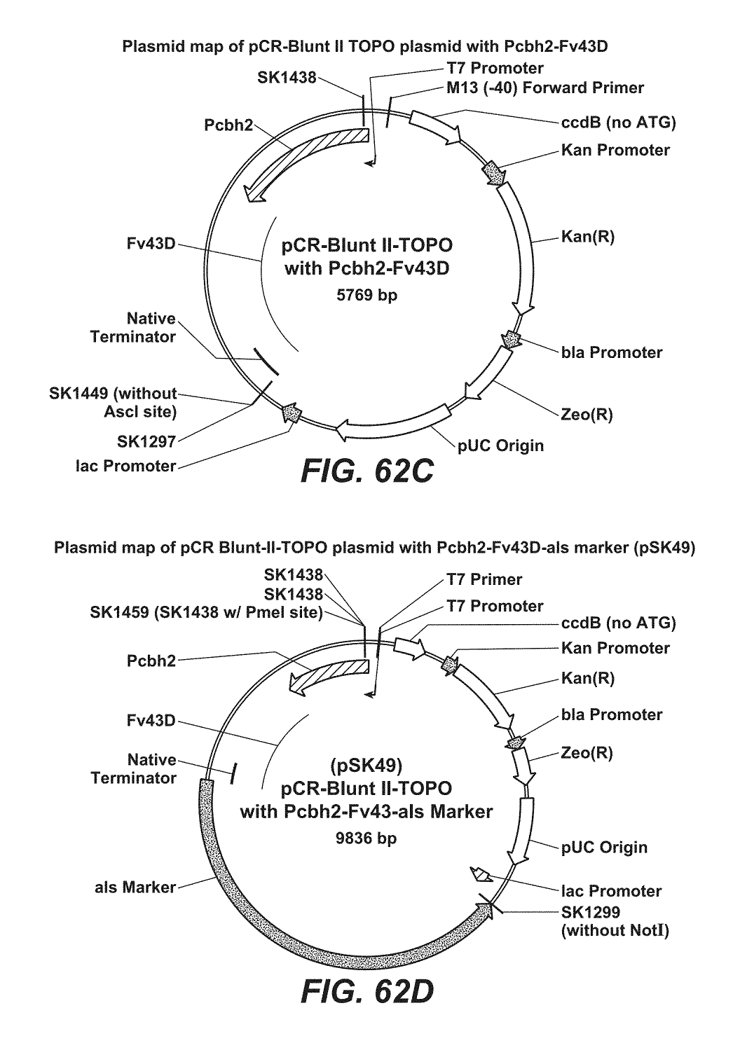

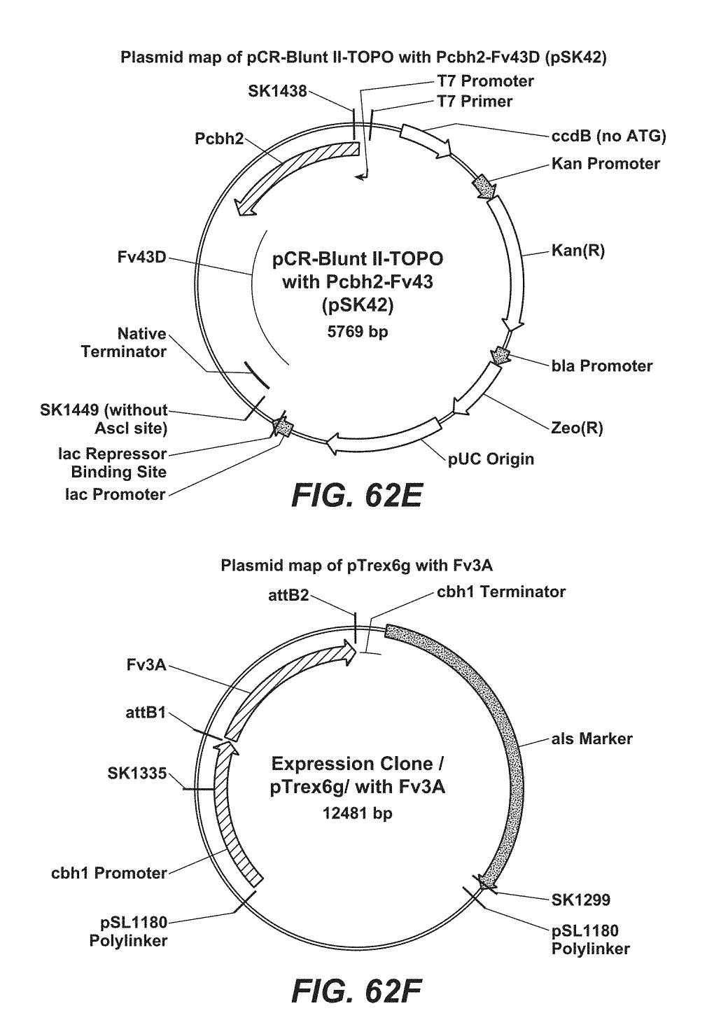

The expression cassettes were amplified from the plasmids shown in FIGS. 62A-62G.

6.23.2 B. Transformation of the CB #201 Strain

The T. reesei CB #201 strain was further transformed by electroporation (WO2009114380) with PCR fragments containing T. reesei eg4 amplified with primers SK1597 and SK1603, T. reesei xyn3 amplified with primers SK1438 and SK1603, and a chimera of Fv3C β-glucosidase from F. verticillioides (fab) amplified with primers RPG159 and RPG163 (see below in Example 23). The selection marker used for the transformations was the amdS gene from A. nidulans, which was contained on the expression cassette amplified by primers RPG159 and RPG163. The transformants were grown on selective media containing acetamide (WO2009114380). Transformants showing stable morphology were cultured in microtiter plates for expression as described in (WO2009114380). Culture supernatants were analyzed by SDS-PAGE and cNPG assay (described above). Select transformants screened for performance in corncob saccharification assays (section F, below).

The following primer pairs were used for amplifying the expression cassettes for transformation of T. reesei:

Peg/1-Tr egl4-cbh1 terminator primer pair:

SK1597
(SEQ ID NO: 162)
5′-GTAGTTATGCGCATGCTAGACTGCTCC-3′
SK1603
(SEQ ID NO: 163)
5′-GCAGGCCGCATCTCCAGTGAAAG-3′

Pcbh2-Tr xyn3-cbh1 terminator primer pair:

| (SEQ ID NO: 164) | |
|---|---|
| SK1438 | 5′- CGTCTAACTCGAACATCTGC -3′ |
| (SEQ ID NO: 165) | |
| SK1603 | 5′- GCAGGCCGCATCTCCAGTGAAAG -3′ |

Pcbh1-fab-cbh1 terminator-amdS primer pair:

| (SEQ ID NO: 166) | |
|---|---|
| RPG159 | 5′- AGTTGTGAAGTCGGTAATCCCGCTGTAT -3′ |
| | (SEQ ID NO: 167) |
| RPG163 | 5′- TCGTAGCATGGCATGGTCACTTCA -3′ |

6.23.3. C. Construction of the Endoxylanase (Xyn3) Expression Cassette

The native T. reesei endoxylanase gene xyn3 (GenBank: BAA89465.2) was amplified by PCR from a genomic DNA sample extracted from a T. reesei strain, using primers xyn3F-2 and xyn3R-2.

Forward Primer (xyn3F-2):
(SEQ ID NO: 168)
5′-CACCATGAAAGCAAACGTCATCTTGTGCCTCCTGG-3′
(where the underlined residues CACC were used to
facilitate cloning into pENTR ™/D-TOPO ®)
Reverse Primer (xyn3R-2):
(SEQ ID NO: 169)
5′-CTATTGTAAGATGCCAACAATGCTGTTATATGCCGGCTTGGGG-3′

The resulting PCR fragments were cloned into the Gateway® vector pENTR™/D-TOPO®, and transformed into E. coli One Shot® TOP10 Chemically Competent cells (Invitrogen) resulting in the intermediate vector, pENTR/Xyn3. The nucleotide sequence of the inserted DNA was determined.

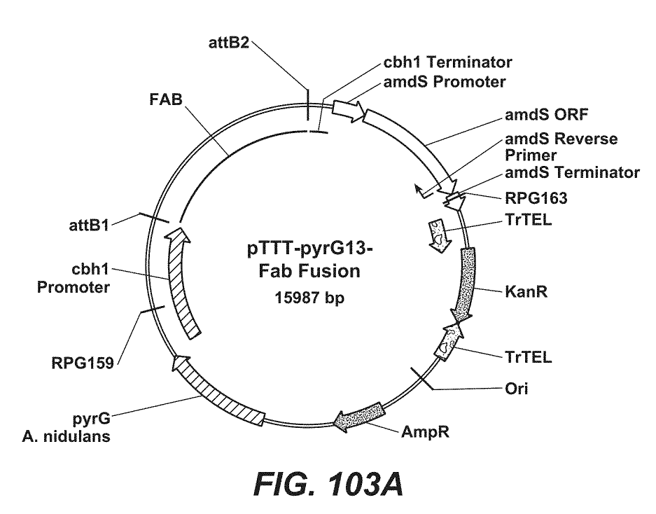

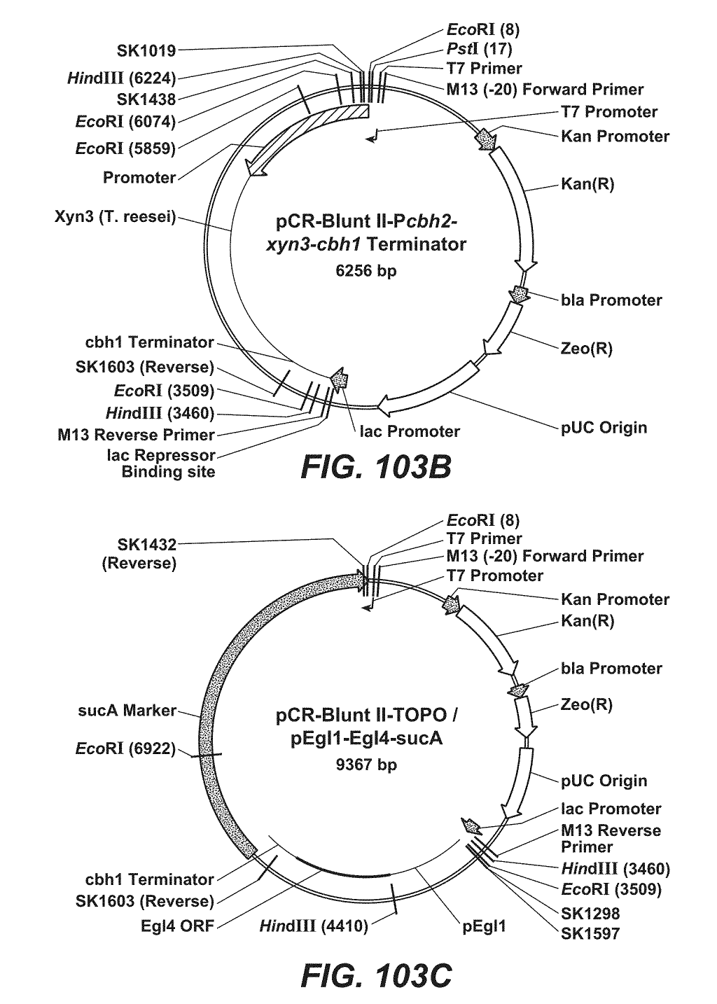

The pENTR/Xyn3 vector with the correct xyn3 sequence was recombined with pTrex3g using the LR clonase® reaction protocol outlined by Invitrogen. The LR clonase reaction mixture was transformed into E. coli One Shot® TOP10 Chemically Competent cells (Invitrogen), resulting in the expression vector, pTrex3g/Xyn3. The vector also contains the Aspergillus nidulans amdS gene, encoding acetamidase, as a selectable marker for transformation of T. reesei. The xyn3 ORF, cbh1 terminator and the amdS sequence were amplified using primers xyn3-F-SOE and SK822. The promoter of cbh2 was amplified with primers SK1019 and cbh2P-R-SOE from genomic DNA of a T. reesei wild-type strain QM6A. Subsequent fusion PCR was performed on the two fragment with primers SK1019 and SK822 to obtain the cassette consisting of Pcbh2-xyn3-and cbh1 terminator. This fusion PCR product was then cloned into pCR-Blunt-II-TOPO (Invitrogen), and transformed into E. coli One Shot® TOP10 Chemically Competent cells (Invitrogen), resulting in the expression vector pCR-Blunt II-TOPO/Pcbh2-xyn3-cbh1 terminator (see, FIG. 103B). The nucleotide sequence of the inserted DNA was confirmed.

Forward Primer (xyn3-F-SOE)
(SEQ ID NO: 170)
5′-AGATCACCCTCTGTGTATTGCACCATGAAAGCAAACGTCA-3′
Reverse Primer (cbh2P-R-SOE)
(SEQ ID NO: 171)
5′-TGACGTTTGCTTTCATGGTGCAATACACAGAGGGTGATCT-3′
Forward Primer (SK1019):
(SEQ ID NO: 172)
5′-GAGTTGTGAAGTCGGTAATCC-3′
Reverse Primer (SK822):
(SEQ ID NO: 173)
5′-CACGAAGAGCGGCGATTC-3′

6.23.4. D. Construction of the Endoglucanase T. Reesei Eg4 Expression Cassette

The native T. reesei endoglucanase gene eg4 (GenBank Accession No. ADJ57703.1) was amplified by PCR from a genomic DNA sample extracted from a T. reesei strain, using primers SK1430 and SK1431.

Forward Primer (SK1430):

| | |
|---|---|
| 5′-CACCATGATCCAGAAGCTTTCCAAC-3′, | (SEQ ID NO: 174) |

wherein the underlined “CACC” were used to to facilitate cloning into pENT™/D-TOPO®.

Reverse Primer (SK1431):

| | |
|---|---|
| 5′-CTAGTTAAGGCACTGGGCGTA-3′ | (SEQ ID NO: 175) |

The resulting PCR fragments were cloned into the Gateway® Entry vector pENTR™/D-TOPO®, and transformed into E. coli One Shot® TOP10 Chemically Competent cells (Invitrogen) resulting in the intermediate vector, pENTR/Egl4. The nucleotide sequence of the inserted DNA was confirmed.

The pENTR/EG4 vector with the correct eg/4 sequence was recombined with pTrex9gM using the LR clonase® reaction protocol outlined by Invitrogen. The LR clonase reaction mixture was transformed into E. coli One Shot® TOP10 Chemically Competent cells (Invitrogen), resulting in the expression vector, pTrex9gM/Egl4. The vector also contains the A. niger sucA gene, encoding sucrase, as a selectable marker for transformation of T. reesei. The egl4 ORF, cbh1 terminator and the sucA sequence was amplified using primers SK1430 and SK1432. The egl1 promoter was PCR amplified from genomic DNA from T. reesei wild-type strain QM6A using primers SK1236 and SK1433. These two DNA fragments were subsequently fused together in a fusion PCR reaction using the primers SK1298 and SK1432. The resulting fusion PCR fragment was cloned into pCR-Blunt II-TOPO vector (Invitrogen) forming TOPO Blunt II-TOPO w/Pegl1-eg14-sucA (see FIG. 103C), and transformed into E. coli One Shot® TOP10 Chemically Competent cells (Invitrogen). The nucleotide sequence of the inserted DNA was confirmed.

Forward Primer (SK1236):
(SEQ ID NO: 176)
5′-CATGCGATCGCGACGTTTTGGTCAGGTCG-3′
Reverse Primer (SK1433):
(SEQ ID NO: 177)
5′-GTTGGAAAGCTTCTGGATCATGGTGTGGGACAACAAGAAGG-3′
Forward Primer (SK1430):
(SEQ ID NO: 178)
5′-CACCATGATCCAGAAGCTTTCCAAC-3′,

wherein the underlined residues were used to facilitate cloning into pENTR™/D-TOPO®)

Reverse Primer (SK1432):

| | |
|---|---|
| 5′-GCTCAGTATCAACCACTAAGC-3′ | (SEQ ID NO: 179) |

Forward Primer (SK1298):

| | |
|---|---|
| 5′-GTAGTTATGCGCATGCTAGAC-3′ | (SEQ ID NO: 180) |

The expression cassette was amplified by PCR with primers SK1597 and SK1603 to generate product for transformation of T. reesei.

Forward Primer (SK1597):

| | |
|---|---|
| 5′-GTAGTTATGCGCATGCTAGACTGCTCC-3′ | (SEQ ID NO: 181) |

Reverse Primer (SK1603):

| | |
|---|---|
| 5′-GCAGGCCGCATCTCCAGTGAAAG-3′ | (SEQ ID NO: 182) |

6.23.5. E. Construction of the B-Glucosidase Chimeric Polypeptide Fv3C/Te3A/T. Reesei Bgl3 Expression Vector

Based on structural data for Fv3C and a predicted model for Bgl3, the fusion between the two molecules was designed at amino acid (aa) position 692 of the full length Fv3C. Namely, the first 1 to 691 aa residues of Fv3C were fused with the region 668-874 aa of Bgl3. The chimeric molecule was constructed using a fusion PCR approach. Entry clones of the genomic Fv3C and Bgl3 coding sequences were used as templates for PCR. Both entry clones were constructed in the pDonor221 vector (Invitrogen, Carlsbad, Calif., USA) according to recommendations of the supplier. The fusion product was assembled in two steps. First, the Fv3C specific sequence was amplified in a PCR reaction using a pEntry Fv3C clone as a template and specific oligonucleotides:

pDonor Forward
(SEQ ID NO: 183)
5′ GCTAGCATGGATGTTTTCCCAGTCACGACGTTGTA
AAACGACGGC-3′;
and
Fv3C/Bgl3 reverse
(SEQ ID NO: 184)
5′ GGAGGTTGGAGAACTTGAACGTCGACCAAGATAGACC
GTGACCGAACTCGTAG-3′

In a similar reaction, the Bgl3 3′ terminal part was amplified from a pENTR Bgl3 vector with the oligonucleotides:

pDonor Reverse:
(SEQ ID NO: 185)
5′-TGCCAGGAAACAGCTATGACCATGTAATACGACTCAC
TATAGG-3′;
and
Fv3C/Bgl3 forward:
(SEQ ID NO: 186)
5′-CTACGAGTTCGGTCACGGTCTATCTTGGTCGACGTTC
AAGTTCTCCAACCTCC-3′.

In the second step, equimolar amounts of each individual PCR product (about 1 μL and 0.2 μL of the initial PCR reactions, respectively) were added as templates for a subsequent fusion PCR reaction using a set of the nested primers:

Att L1 for
(SEQ ID NO: 187)
5′TAAGCTCGGGCCCCAAATAATGATTTTATTTTGACTGATAGT-3′;
and
AttL2 rev
(SEQ ID NO: 188)
5′GGGATATCAGCTGGATGGCAAATAATGATTTTATTTTGACTGATA-3′

All PCR reactions were performed using a high fidelity Phusion DNA polymerase (Finnzymes OY, Espoo, Finland) under standard conditions recommended by the supplier. The final PCR product fused contained the intact Gateway-specific attL1, attL2 recombination sites on both ends allowing for direct cloning into a final destination vector via a Gateway LR recombination reaction (Invitrogen, Carlsbad, Calif., USA).

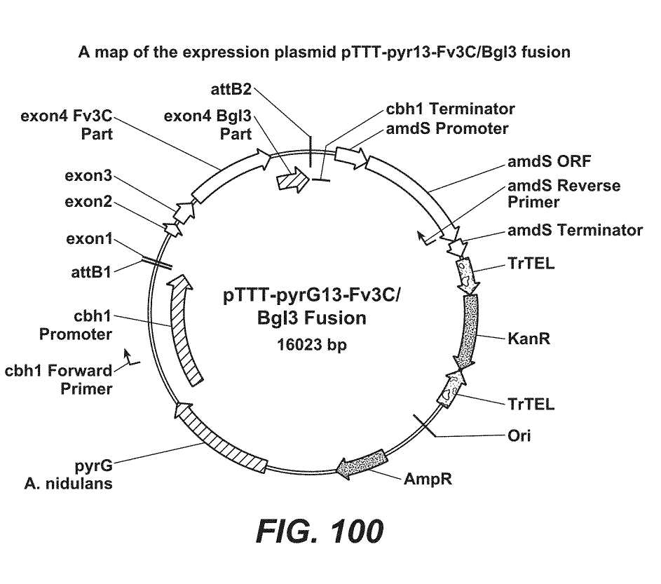

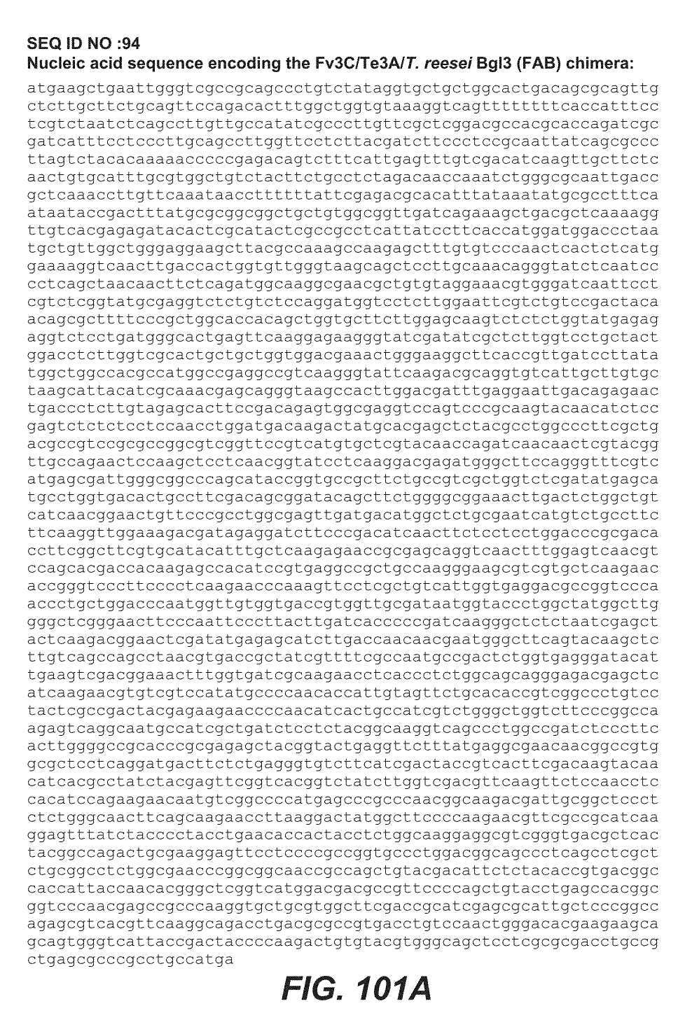

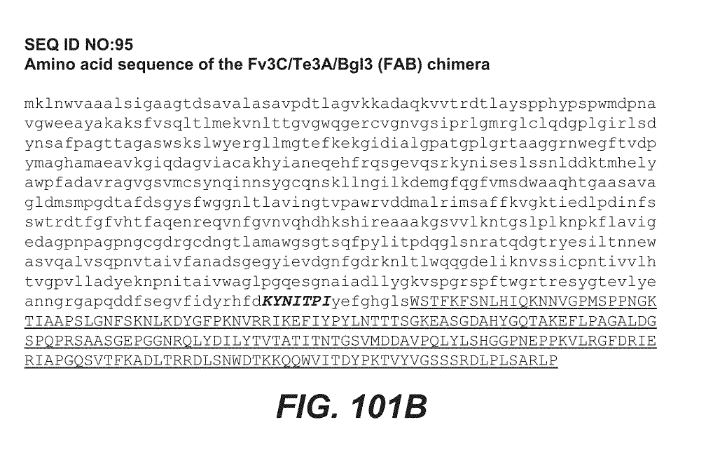

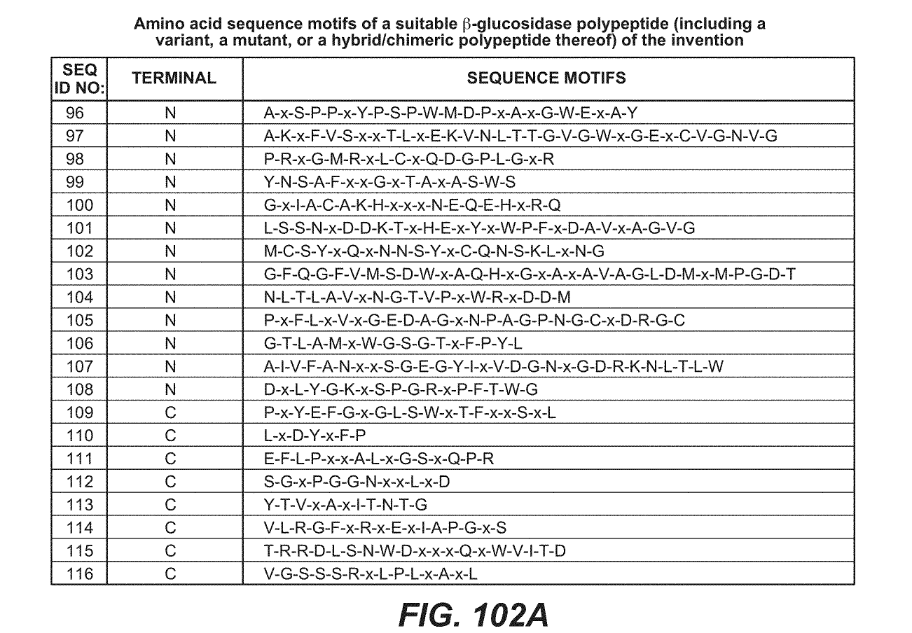

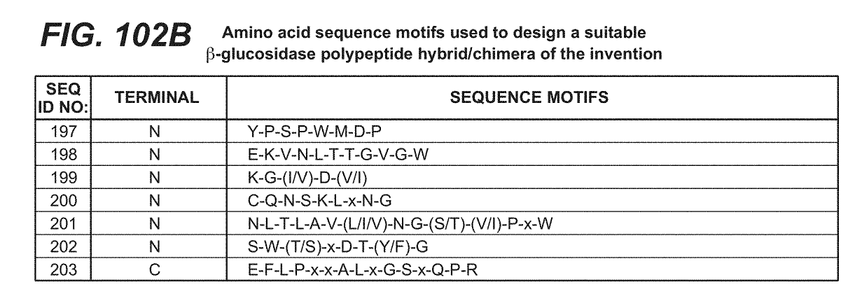

After separation of the specific DNA fragment on a 0.8% agarose gel, it was purified with a Nucleospin® Extract PCR clean-up kit (Macherey-Nagel GmbH & co. KG, Duren, Germany) and 100 ng were recombined with of the pTTT-pyrG13 (see, International Patent Application Publication WO2009/048488) destination vector using the LR clonase™ II enzyme mix according to the protocol from Invitrogen. Recombination products generated were transformed to E. coli Max Efficiency DH5α, as described by the supplier (Invitrogen), and clones containing the expression construct pTTT-pyrG13-Fv3C/Bgl3 fusion (FIG. 100) with the chimeric β-glucosidase were selected on 2× YT agar plates (16 g/L Bacto Tryptone (Difco, USA), 10 g/L Bacto Yeast Extract (Difco, USA), 5 g/L NaCl, 16 g/L Bacto Agar (Difco, USA)) with 100μg/ml ampicillin. After growth of bacterial cultures in 2× YT medium with 100μg/ml ampicillin, isolated plasmids were subjected to restriction analysis with either BglI or EcoRV restriction enzymes and the Fv3C/Bgl3 (“FB”) specific region was sequenced using a AB13100 sequence analyzer (Applied Biosystems).

Two N-glycosylation sites, S725N and S751N, were introduced into the Bgl3-derived part of the chimera. Equivalent positions are glycosylated in Fv3C but not in Bgl3. The glycosylation mutations were introduced in the Fv3C/Bgl3 (FB) backbone essentially via the same PCR fusion approach with the exception that the pTTT-pyrG13-Fv3C/Bgl3 fusion plasmid (FIG. 100) was used as a template for the first PCR reactions, as described previously. One PCR product was generated using the primers:

Pr Cbhl forward:
(SEQ ID NO: 189)
5′ CGGAATGAGCTAGTAGGCAAAGTCAGC-3′;
and
725/751 reverse:
(SEQ ID NO: 190)
5′-CTCCTTGATGCGGCGAACGTTCTTGGGGAAGCCATAGTCCTTAAG
GTTCTTGCTGAAGTTGCCCAGAGAG-3′

The second PCR fragment was amplified using a set oligonucleotides:

725/751 forward:
(SEQ ID NO: 191)
5′-
GGCTTCCCCAAGAACGTTCGCCGCATCAAGGAGTTTATCTACCCCTA
CCTGAACACCACTACCTC-3′;
and
Ter Cbhl reverse:
(SEQ ID NO: 192)
5′ GATACACGAAGAGCGGCGATTCTACGG-3′

Finally, both PCR fragments obtained were fused together using primers Pr Cbhl forward and Ter Cbhl reverse as described above. The fusion product with two glycosylation mutations introduced contained the attB1 and attB2 sites allowing for recombination with the pDonor221 vector using the Gateway BP recombination reaction (Invitrogen, Carlsbad, Calif., USA) according to recommendation of the supplier. E. coli DH5α colonies with pENTR clones containing the Fv3C/Bgl3 chimeric β-glucosidase with two extra glycosylation mutations S725N S751N were selected on 2× YT agar plates with 50 μg/ml kanamycin. Plasmids isolated from bacterial cells were analyzed by their restriction digestion pattern for the insert presence and mutations were checked by sequence analysis using an AB13100 sequence analyzer (Applied Biosystems). This resulted in the pEntry-Fv3C/Bgl3/S725N S751N clone which was used for further modifications.

Amino acid residues 665 to 683 of the Fv3C/Bgl3 hybrid above were replaced with a corresponding sequence from Talaromyces emersonii, resulting in a fusion/chimera Fv3C/Te3A/Bgl3/S713N S739N (for plasmid used, see, FIG. 103A). To introduce the T. emersonii β-glucosidase sequence, referred to as Te3A (SEQ ID NO: 66) the first PCR reactions were performed using the following sets of primers:

Set 1:

pDonor Forward:
(SEQ ID NO: 193)
5′-GCTAGCATGGATGTTTTCCCAGTCACGACGTTGTAAA
ACGACGGC-3′;
and
ABG2 reverse:
(SEQ ID NO: 194)
5′-GATAGACCGTGACCGAACTCGTAGATAGGCGTGATGTTGTAC
TTGTCGAAGTGACGGTAGTCGATGAAGAC-3′;

Set 2:

ABG2 forward:
(SEQ ID NO: 195)
5′- GTCTTCATCGACTACCGTCACTTCGACAAGTACAACATCACGC
CTATCTACGAGTTCGGTCACGGTCTATC-3′;
and
pDonor Reverse:
(SEQ ID NO: 196)
5′ TGCCAGGAAACAGCTATGACCATGTAATACGACTCACTA TAGG-3′

6.23.6. F. Screening Procedure for Biomass

Screening of transformants for biomass performance was performed on microtiter plate scale using dilute ammonia pretreated corncob. The pretreated corncob was suspended with water and adjusted to pH 5.0 with sulfuric acid to 8.7% cellulose (25.2% solids). The slurry was dispensed (70 mg/well) into a flat bottom 96-well microtiter plate (Nunc) and centrifuged at 3,000 rpm for 5 min. The transformant strains were grown in shake flask format. The new strains were assayed by SDS-PAGE to check for expression levels prior to incubation with the corncob substrate. The total protein of each sample was determined and samples were diluted to 2 mg/mL.

Corncob saccharification reactions were initiated by adding 5, 10, 20, or 30 μL of strain product per corncob well. Following this format, a broad dose-response of transformed strain products were generated on the corncob substrate.

The corncob saccharification reactions were sealed with aluminum plate seals (E&K scientific) and mixed for 1 minute at 450 rpm, room temperature. The plate was then placed in an Innova incubator at 50° C. and 200 rpm for 72 h.

At the end of the 72-h saccharification step, the plate was quenched by adding 100 μL of 100 mM glycine, pH 10.0. The plate was then mixed thoroughly and centrifuged at 3,000 rpm for 5 min (Rotanta 460R Centrifuge from Hettich Zentrifugen).

Supernatant (10 μL) was added to 100 μL of water in an HPLC 96-well microtiter plate (Agilent, 5042-1385). Glucose, xylose, cellobiose and xylobiose concentrations were measured by HPLC using Aminex HPX-87P column (300 mm×7.8 mm, 125-0098) pre-fitted with guard column.

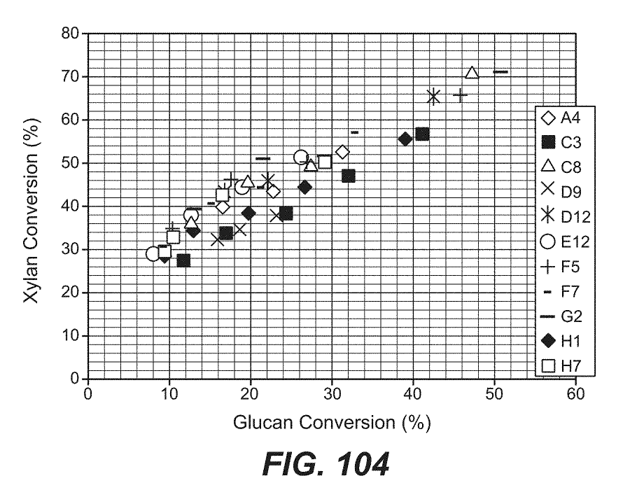

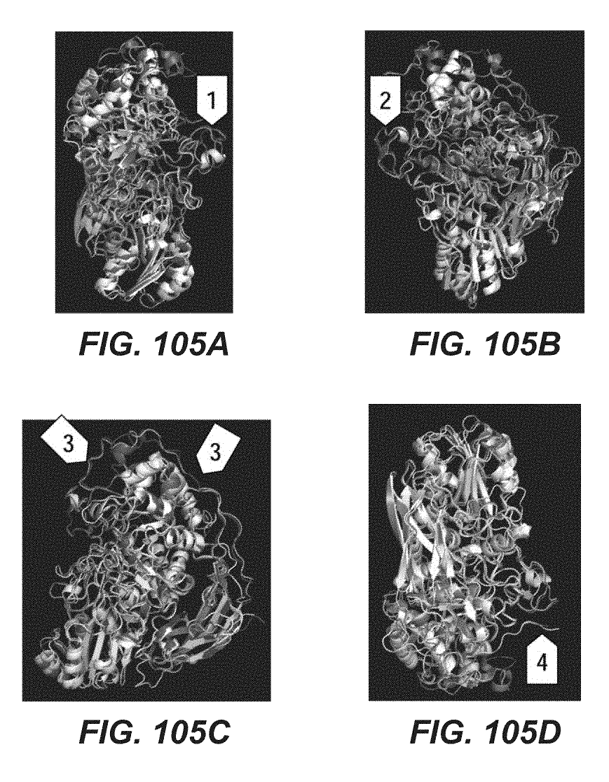

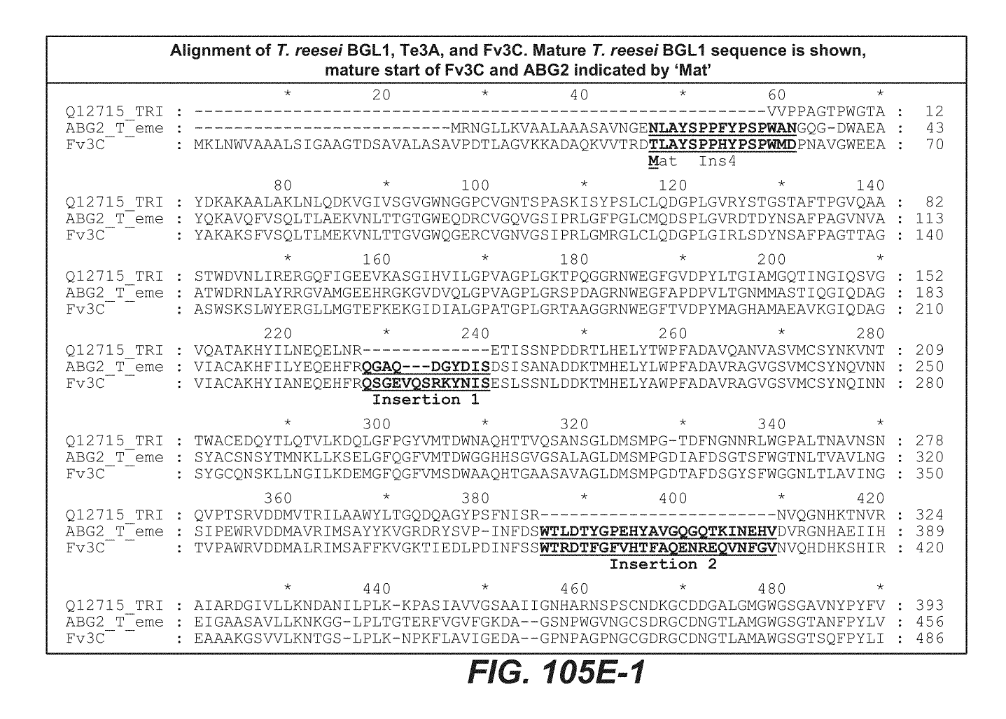

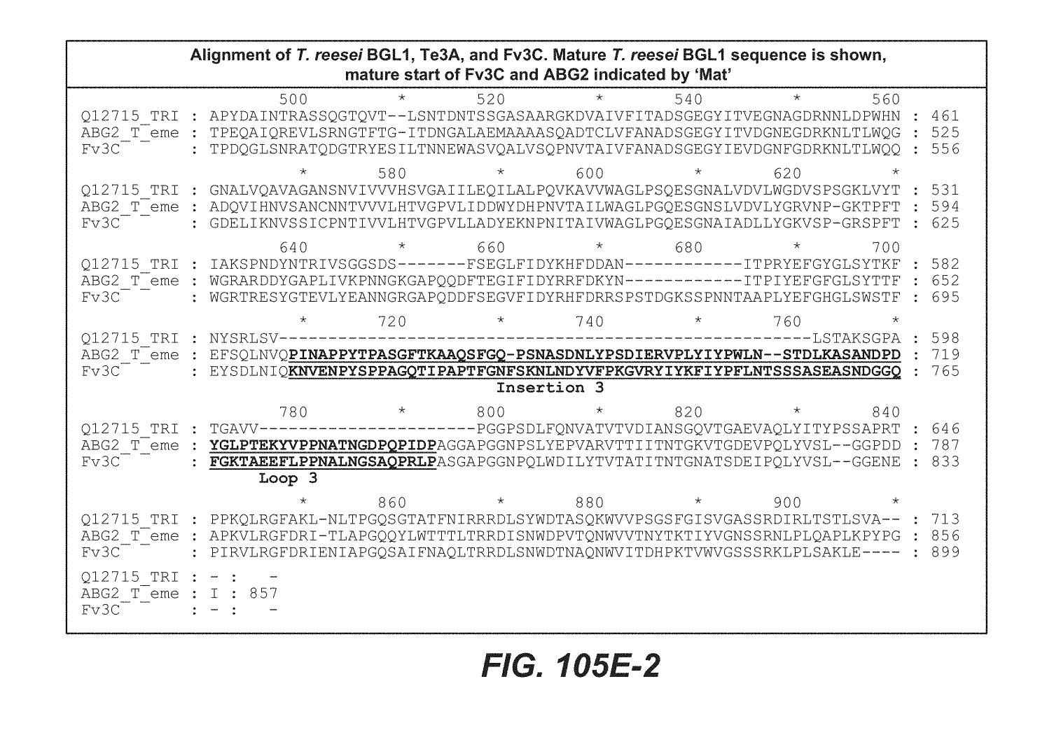

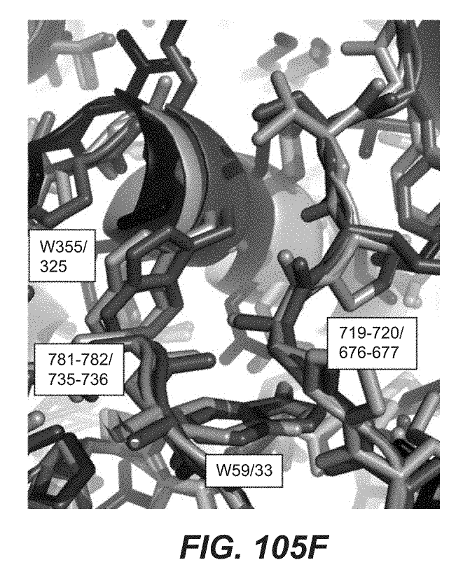

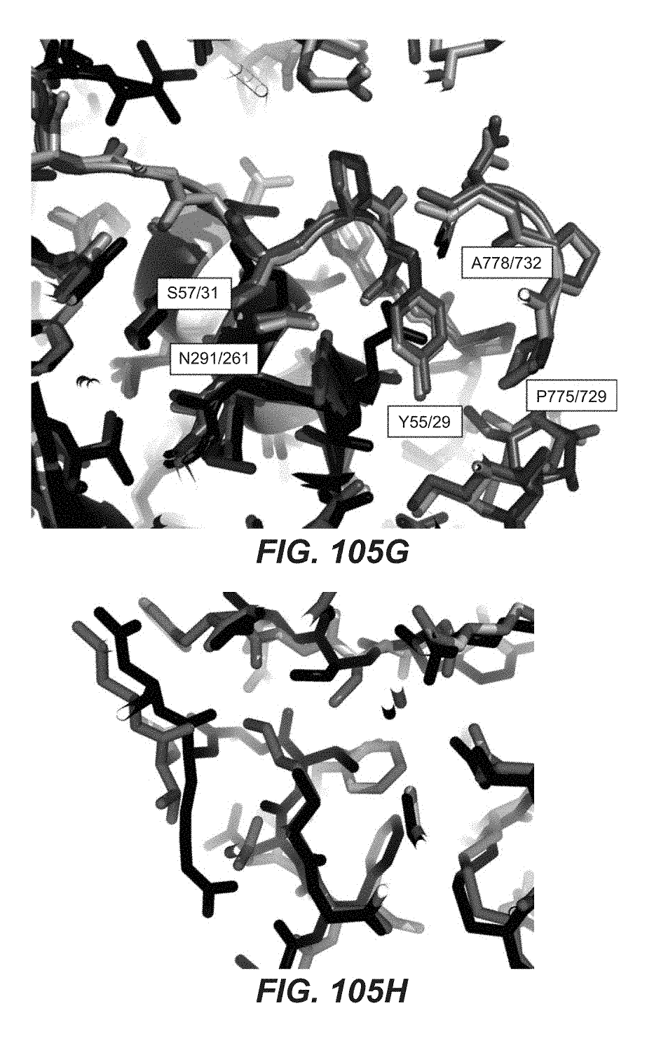

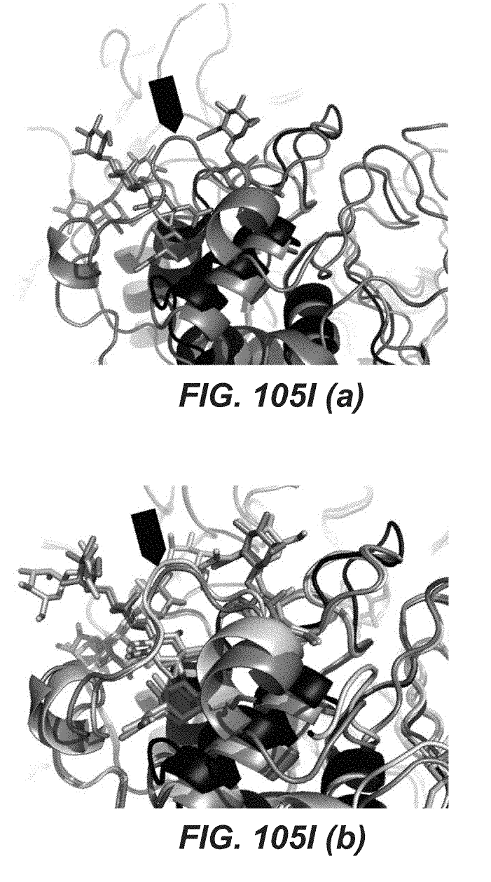

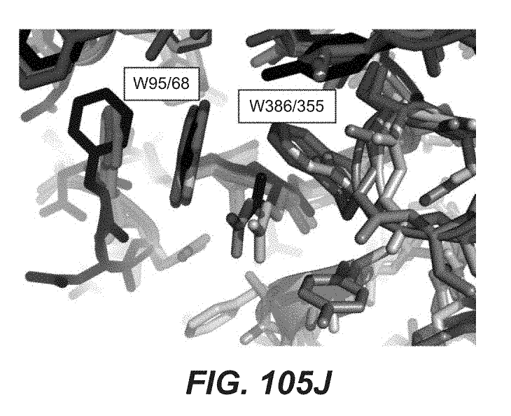

The performance of eleven strains: A4, C3, C8, D9, D12, E12, F5, F7, G2, H1, H7 are depicted in FIG. 104. Glucan (cellobiose and glucose) and xylan (xylobiose+xylose) conversions of these strains are shown.

### Example 24

### Protein Quantitation of Enzyme Compositions Using UPLC

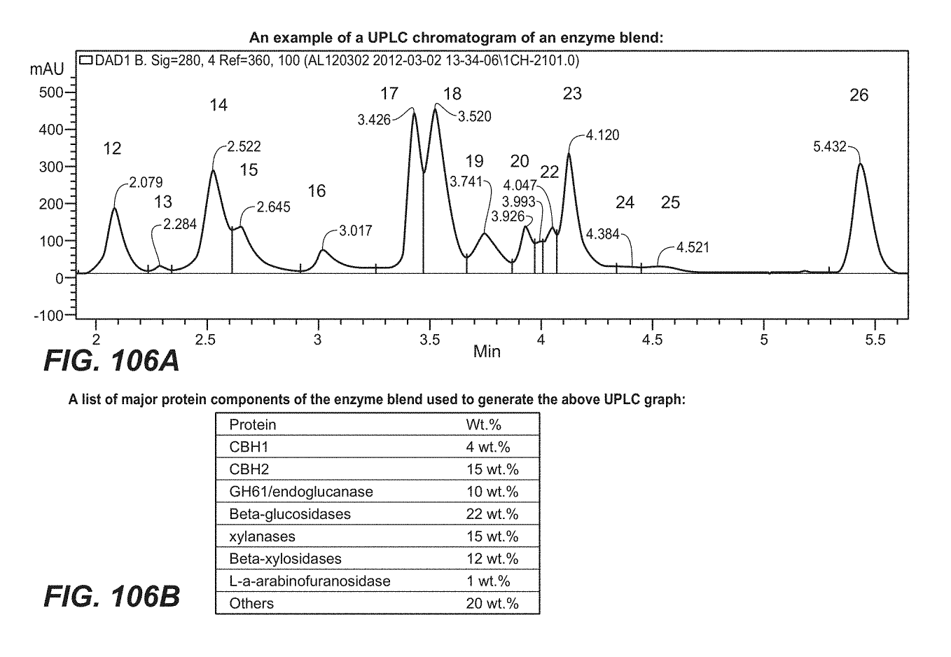

An Agilent HPLC 1290 Infinity system for protein quantitation. A Waters ACQUITY UPLC BEH C4 Column (1.7 μm, 1×50 mm) was used. A 6-min program with an initial gradient from 5% to 33% acetonitrile (Sigma-Aldrich) in 0.5 mins, followed by a gradient from 33% to 48% in 4.5 mins, and then a step gradient to 90% acetronitrile was used. The proteins of interest were eluted between 33% to 48% acetonitrile. Retention times of purified proteins such as CBH1, CBH2, endoglucanases, xylanases, beta-glucosidases, etc., were used as standards. Based on peak area of each protein in any enzyme blends, the percent of each protein vis-a-vis the total proteins in that blend was calculated. An example of an enzyme blend used herein is presented as FIGS. 106A-B.

We claim:

1. An engineered enzyme composition, comprising:
a) a polypeptide having xylanase activity; and
b) a polypeptide having β-xylosidase activity selected from a Group 1 or 2 β-xylosidase; and
c) a polypeptide having L-α-arabinofuranosidase activity; and
d) a polypeptide having β-glucosidase activity or a whole cellulase enriched with the polypeptide having β-glucosidase activity,
wherein the enzyme composition is capable of hydrolyzing a lignocellulosic biomass material.

2. An engineered enzyme composition comprising:
a) a polypeptide having β-xylosidase activity selected from a Group 1 β-xylosidase; and
b) a polypeptide having β-xylosidase activity selected from a Group 2 β-xylosidase; and
c) a polypeptide having L-α-arabinofuranosidase activity; and
d) a polypeptide having β-glucosidase activity or a whole cellulase enriched with the polypeptide having β-glucosidase activity,
wherein the enzyme composition is capable of hydrolyzing a lignocellulosic biomass material.

3. An engineered enzyme composition comprising:
a) a polypeptide having xylanase activity; and
b) a polypeptide having β-xylosidase activity selected from a Group 1 β-xylosidase; and
c) a polypeptide having β-xylosidase activity selected from a Group 2 β-xylosidase; and
d) a polypeptide having β-glucosidase activity or a whole cellulase enriched with the polypeptide having β-glucosidase activity,
wherein the enzyme composition is capable of hydrolyzing a lignocellulosic biomass material.

4. An engineered enzyme composition comprising:
a) a polypeptide having xylanase activity; and
b) a polypeptide having β-xylosidase activity selected from a Group 1 or 2 β-xylosidase; and
c) a polypeptide having β-glucosidase activity or a whole cellulase enriched with the polypeptide having β-glucosidase activity,
wherein the enzyme composition is capable of hydrolyzing a lignocellulosic biomass material.

5. The enzyme composition of any one of claims 1-4, further comprising a polypeptide having GH61/endoglucanase activity or a whole cellulase enriched with the polypeptide having GH61/endoglucanase activity

6. An engineered enzyme composition, comprising:
a) a polypeptide having xylanase activity; and
b) a polypeptide having β-xylosidase activity selected from a Group 1 or 2 β-xylosidase; and
c) a polypeptide having L-α-arabinofuranosidase activity; and
d) a polypeptide having GH61/endoglucanase activity or a whole cellulase enriched with the polypeptide having GH61/endoglucanase activity,
wherein the enzyme composition is capable of hydrolyzing a lignocellulosic biomass material.

7. An engineered enzyme composition comprising:
a) a polypeptide having β-xylosidase activity selected from a Group 1 β-xylosidase; and
b) a polypeptide having β-xylosidase activity selected from a Group 2 β-xylosidase; and
c) a polypeptide having L-α-arabinofuranosidase activity; and
d) a polypeptide having GH61/endoglucanase activity or a whole cellulase enriched with the polypeptide having GH61/endoglucanase activity,
wherein the enzyme composition is capable of hydrolyzing a lignocellulosic biomass material.

8. An engineered enzyme composition comprising:
a) a polypeptide having xylanase activity; and
b) a polypeptide having β-xylosidase activity selected from a Group 1 β-xylosidase; and
c) a polypeptide having β-xylosidase activity selected from a Group 2 β-xylosidase; and
d) a polypeptide having GH61/endoglucanase activity or a whole cellulase enriched with the polypeptide having GH61/endoglucanase activity,
wherein the enzyme composition is capable of hydrolyzing a lignocellulosic biomass material.

9. An engineered enzyme composition comprising:
a) a polypeptide having xylanase activity; and
b) a polypeptide having β-xylosidase activity selected from a Group 1 or 2 β-xylosidase; and
c) a polypeptide having GH61/endoglucanase activity or a whole cellulase enriched with the polypeptide having GH61/endoglucanase activity,
wherein the enzyme composition is capable of hydrolyzing a lignocellulosic biomass material.

10. The engineered enzyme composition of any one of claims 1-9, wherein the polypeptide having xylanase activity is: selected from a polypeptide comprising an amino acid sequence that has at least 70% identity to SEQ ID NO: 24, 26, 42, or 43, or to a mature sequence thereof; or encoded by a nucleotide having at least 70% identity to SEQ ID NO:23, 25, or 41, or by a nucleotide that is capable of hybridizing under high stringency condition to SEQ ID NO: 23, 25 or 41, or to a complement thereof.

11. The engineered enzyme composition of any one of claims 1-10, wherein:
a) the polypeptide having β-xylosidase activity of Group 1 comprises an amino acid sequence having at least 70% identity to SEQ ID NO: 2 or 10 or to a mature sequence thereof, and the polypeptide having β-xylosidase activity of Group 2 comprises an amino acid sequence having at least 70% to SEQ ID NO: 4, 6, 8, 10, 12, 14, 16, 18, 28, 30, or 45, or to a mature sequence thereof; or
b) the polypeptide having β-xylosidase activity of Group 1 is encoded by a nucleotide comprises an amino acid sequence having at least 70% identity to SEQ ID NO: 2 or 10 or to a mature sequence thereof, and the polypeptide having β-xylosidase activity of Group 2 comprises an amino acid sequence having at least 70% to SEQ ID NO: 4, 6, 8, 10, 12, 14, 16, 18, 28, 30, or 45, or to a mature sequence thereof; or
c) the polypeptide having β-xylosidase activity of Group 1 encoded by a nucleotide having at least 70% identity to SEQ ID NO:1 or 9; and the polypeptide having β-xylosidase activity of Group 2 encoded by a nucleotide having at least 70% identity to SEQ ID NO:3, 5, 7, 9, 11, 13, 15, 17, 27, or 29; or
d) the polypeptide having β-xylosidase activity of Group 1 capable of hybridizing under high stringency conditions to SEQ ID NO:1 or 9, or to a complement thereof; and the polypeptide having β-xylosidase activity of Group 2 capable of hybridizing under high stringency conditions to SEQ ID NO:3, 5, 7, 9, 11, 13, 15, 17, 27, or 29, or to a complement thereof.

12. The engineered enzyme composition of any one of claims 1-11, wherein the polypeptide having L-α-arabinofuranosidase activity is:
a) a polypeptide comprising an amino acid sequence that has at least 70% identity to SEQ ID NO:12, 14, 20, 22 or 32, or to a mature sequence thereof; or
b) a polypeptide encoded by a nucleotide having at least 70% identity to SEQ ID NO:11, 13, 19, 21, or 31, or a nucleotide capable of hybridizing under high stringency conditions to SEQ ID NO: SEQ ID NO:11, 13, 19, 21, or 31.

13. The engineered enzyme composition of any one of claims 1-12, wherein the polypeptide having β-glucosidase activity is:
a) a polypeptide comprising an amino acid sequence having at least about 60% identity to SEQ ID NO: 54, 56, 58, 60, 62, 64, 66, 68, 70, 72, 74, 76, 78, 79, 93, and 95; or
b) a hybrid polypeptide comprising 2 or more β-glucosidase sequences, wherein the first sequence derived from a first β-glucosidase is at least 200 amino acid residues in length and comprises one or more or all of SEQ ID NOs: 96-108, and the second sequence derived from a second β-glucosidase is at least 50 amino acid residues in length and comprises one or more or all of SEQ ID NOs: 109-116, and optionally a third sequence derived from a third β-glucosidase of 3, 4, 5, 6, 7, 8, 9, 10, or 11 amino acid residues in length encoding a loop sequence comprising SEQ ID NO: 204 or 205; or
c) a polypeptide encoded by a nucleotide that has at least about 60% identity to SEQ ID NO: 53, 55, 57, 59, 61, 63, 65, 67, 69, 71, 73, 75, 77, 92 or 94, or one that is capable of hybridizing under high stringency conditions to SEQ ID NO: 53, 55, 57, 59, 61, 63, 65, 67, 69, 71, 73, 75, 77, 92 or 94, or to a complement thereof.

14. The engineered enzyme composition of any one of claims 1-13, wherein the polypeptide having GH61/endoglucanase activity is:
a) a polypeptide comprising an amino acid sequence having at least 70% sequence identity to any one of SEQ ID NOs:52, 80-81, 206-207, over a region of at least 100 residues; or
b) a polypeptide that is at least 200 residues in length, having GH61/endoglucanase activity, and comprising one or more sequence selected from the group consisting of: (1) SEQ ID NOs:84 and 88; (2) SEQ ID NOs:85 and 88; (3) SEQ ID NO:86; (4) SEQ ID NO:87; (5) SEQ ID NOs:84, 88 and 89; (6) SEQ ID NOs:85, 88, and 89; (7) SEQ ID NOs: 84, 88, and 90; (8) SEQ ID NOs: 85, 88 and 90; (9) SEQ ID NOs:84, 88 and 91; (10) SEQ ID NOs: 85, 88 and 91; (11) SEQ ID NOs: 84, 88, 89 and 91; (12) SEQ ID NOs: 84, 88, 90 and 91; (13) SEQ ID NOs: 85, 88, 89 and 91: and (14) SEQ ID NOs: 85, 88, 90 and 91; or
c) a polypeptide encoded by a nucleotide having at least 70% sequence identity to SEQ ID NO:51, or is capable of hybridizing under high stringency conditions to SEQ ID NO:51 or to a complement thereof.

15. The engineered enzyme composition of any one of claims 1-14, wherein the polypeptide having β-glucosidase activity is a hybrid polypeptide comprising 2 or more β-glucosidase sequences, wherein the first sequence derived from a first β-glucosidase is at least 200 amino acid residues in length and comprises one or more or all of SEQ ID NOs: 197-202, and the second sequence derived from a second β-glucosidase is at least 50 amino acid residues in length and comprises SEQ ID NO:203, and optionally a third polypeptide sequence of 3-11 amino acid residues in length comprising SEQ ID NO:204 or SEQ ID NO:205.

16. The engineered enzyme composition of any one of claims 1-15, which is a culture mixture, a fermentation broth of a host cell expressing one or more of the polypeptides, or a whole broth formulation of the fermentation broth.

17. The engineered enzyme composition of claim 16, wherein the host cell is one of a bacterium or a fungus.

18. The engineered enzyme composition of claim 17, wherein the bacterium is a Bacillus, or an E. coli.

19. The engineered enzyme composition of claim 17, wherein the fungus is a yeast, an Aspergillus, a Chrysosporium, or a Trichoderma.

20. The engineered enzyme composition of any one of claims 1-19, further comprising a polypeptide having cellolubiohydrolase activity and/or a polypeptide having endoglucanase activity.

21. The engineered enzyme composition of any one of claims 1-19, further comprising a whole cellulase.

22. The engineered enzyme composition of any one of claims 1-21, wherein the amount of xylanase relative to the total amount of proteins in the enzyme composition is about 10 wt. % to about 20 wt. %.

23. The engineered enzyme composition of any one of claims 1-21, wherein the amount of β-xylosidase relative to the total amount of proteins in the enzyme composition is about 5 wt. % to about 20 wt. %.

24. The engineered enzyme composition of any one of claims 1-23, wherein the amount of β-glucosidase relative to the total amount of proteins in the enzyme composition is about 18 wt. % to about 30 wt. %.

25. The engineered enzyme composition of any one of claims 1-24, wherein the amount of L-α-arabinofuranosidase relative to the total amount of proteins in the enzyme composition is about 0.2 wt. % to about 2 wt. %.

26. The engineered enzyme composition of any one of claims 1-25, wherein the amount of polypeptides having GH61/endoglucanase activity relative to the total amount of proteins in the enzyme composition is about 6 wt. % to about 20 wt. %.

27. The engineered enzyme composition of any one of claims 1-26, wherein the amount of polypeptides having cellobiohydrolase activity relative to the total amount of proteins in the enzyme composition is about 15 wt. % to about 25 wt. %.

28. The engineered enzyme composition of any one of claims 2-5, 7-8, and 10-27, wherein the ratio of the weight of Group 1 β-xylosidase to the weight of Group 2 β-xylosidase is 1:10 to 10:1, 1:9 to 9:1, 1:8 to 8:1, 1:7 to 7:1, 1:6 to 6:1, 1:5 to 5:1, 1:4 to 4:1, 1:3 to 3:1, 1:2 to 2:1, or 1:1.

29. The engineered enzyme composition of any one of claims 1-28, wherein at least 1, 2, or 3 of the polypeptides are heterologous to the host cell engineered to express the polypeptides.

30. The engineered enzyme composition of any one of claims 1-28, wherein at least 2 of the polypeptides are derived from different microorganisms.

31. The engineered enzyme composition of claim 30, wherein at least one of the polypeptides are from a Fusarium, or a Trichoderma.

32. A method of hydrolyzing or digesting a lignocellulosic biomass material comprising hemicelluloses, cellulose, or both cellulose and hemicelluloses, comprising contacting the enzyme composition of any one of claims 1-31 with the lignocellulosic biomass mixture.

33. The method of claim 32, wherein the lignocellulosic biomass mixture comprises an agricultural crop, a byproduct of a food/feed production, a lignocellulosic waste product, a plant residue, or waste paper.

34. The method of claim 33, wherein the plant reside is selected from grain, seeds, sterns, leaves, hulls, husks, corncobs, corn stover, potatos, soybean, barley, rye, oats, wheat, beats, sugarcane bagasse, sorghum, straw, grasses, canes, reeds, wood, wood chips, wood pulp, or sawdust.

35. The method of claim 33, wherein the grass is selected from Indian grass or switchgrass.

36. The method of claim 32, wherein the biomass material in the lignocellulosic biomass mixture is subjected to pretreatment.

37. The method of any one of claims 32-36, wherein the lignocellulosic biomass mixture further comprises a fermentable sugar.

38. The method of claim 36, wherein the pretreatment is an acidic or a basic pretreatment.

39. The method of claim 38, wherein the basic pretreatment is with a dilute ammonia,

40. The method of claim 38, wherein the acidic pretreatment is with a dilute acid.

41. A method of producing ethanol comprising contacting a lignocellulosic biomass material with an enzyme composition of any one of claims 1-31 to produce one or more fermentable sugar, followed by fermenting the fermentable sugar into ethanol using an ethanologen microorganism.

42. The method of claim 41, wherein the lignocellulosic biomass material is subjected to pretreatment before it contacts the enzyme composition.

43. The method of claim 41 or 42, wherein the ethanologen microorganism is a yeast, or a Zymomonas mobilis.

44. The method of any one of claims 32-43, wherein the enzyme composition comprises about 2 g to about 20 g of polypeptide having xylanase activity per kilogram of hemicelluloses in the biomass material.

45. The method of any one of claims 32-44, wherein the enzyme composition comprises about 2 g to about 40 g of polypeptide having β-xylosidase activity per kilogram of hemicelluloses in the biomass material.

46. The method of any one of claims 32-45, wherein the enzyme composition comprises about 3 g to about 50 g of polypeptide having cellulase activity per kilogram of cellulose in the biomass material.

47. The method of claim 46, wherein the amount of polypeptide having β-glucosidase activity constitutes up to about 50% of the total weight of polypeptide having cellulase activity.

48. The method of any one of claims 32-47, wherein the enzyme composition is used in an amount, and under conditions and for a duration sufficient to convert 60% to 90% of the xylan in the biomass material into xylose.

49. A method of using the enzyme composition of any one of claims 1-31 in an industrial or commercial setting following a merchant enzyme supply model strategy or a on-site biorefinery model strategy.
